(19) United States
(12) Patent Application Publication
(10) Pub. No.: US 2015/0024961 A1
(43) Pub. Date: Jan. 22, 2015

(54) METHODS AND SYSTEMS OF USING BIOMARKERS FOR DETERMINING PHENOTYPES

(71) Applicant: Caris Life Sciences Switzerland Holdings GmbH, Basel (CH)

(72) Inventors: Michael KLASS, Oro Valley, AZ (US); Christine KUSLICH, Laguna Beach, CA (US); George POSTE, Cave Creek, AZ (US)

(21) Appl. No.: 14/444,872

(22) Filed: Jul. 28, 2014

Related U.S. Application Data
(63) Continuation-in-part of application No. 13/459,016, filed on Apr. 27, 2012, now abandoned.
Application No. 13/459,016 is a continuation-in-part of application No. 12/963,468, filed on Dec. 8, 2010, now Pat. No. 8,192,954.
Application No. 12/963,468 is a continuation of application No. 12/609,847, filed on Oct. 30, 2009, now Pat. No. 7,888,035.
Application No. 13/459,016 is a continuation-in-part of application No. 13/009,285, filed on Jan. 19, 2011, now Pat. No. 8,211,653.
Application No. 13/009,285 is a continuation of application No. 12/658,452, filed on Feb. 5, 2010, now abandoned.
Application No. 12/658,452 is a continuation of application No. 12/591,226, filed on Nov. 12, 2009, now Pat. No. 7,897,356.
(60) Provisional application No. 61/109,742, filed on Oct. 30, 2008.
(60) Provisional application No. 61/112,571, filed on Nov. 7, 2008.
(60) Provisional application No. 61/114,045, filed on Nov. 12, 2008.
(60) Provisional application No. 61/114,058, filed on Nov. 12, 2008.
(60) Provisional application No. 61/114,065, filed on Nov. 13, 2008.
(60) Provisional application No. 61/151,183, filed on Feb. 9, 2009.
(60) Provisional application No. 61/278,049, filed on Oct. 2, 2009.
(60) Provisional application No. 61/250,454, filed on Oct. 9, 2009.
(60) Provisional application No. 61/253,027, filed on Oct. 19, 2009.
(60) Provisional application No. 61/114,045, filed on Nov. 12, 2008.
(60) Provisional application No. 61/114,058, filed on Nov. 12, 2008.
(60) Provisional application No. 61/114,065, filed on Nov. 13, 2008.
(60) Provisional application No. 61/151,183, filed on Feb. 9, 2009.
(60) Provisional application No. 61/278,049, filed on Oct. 2, 2009.
(60) Provisional application No. 61/250,454, filed on Oct. 9, 2009.
(60) Provisional application No. 61/253,027, filed on Oct. 19, 2009.

(51) Int. Cl. C12Q 1/68; G01N 33/574

(52) U.S. Cl. CPC C12Q 1/6886; G01N 33/57496; C12Q 2600/178
USPC 506/9; 435/7.1; 435/6.12; 435/7.92

(57) ABSTRACT

Biomarkers such as exosomes and/or RNA, e.g., microRNA, can be used for diagnostic, therapy-related or prognostic methods to identify phenotypes, such as a condition or disease, for example, the stage or progression of a disease. Such biomarkers can be used in profiling of physiological states or determining phenotypes. Biomarkers can be used to determine treatment regimens for diseases, conditions, disease stages, and stages of a condition, and can also be used to determine treatment efficacy. Biomarkers can also be used to identify conditions of diseases of unknown origin.

### CROSS-REFERENCE

This application is a continuation-in-part of U.S. patent application Ser. No. 13/459,016, filed Apr. 27, 2012; which application is a continuation-in-part of U.S. patent application Ser. No. 12/963,468, filed Dec. 8, 2010 and now U.S. Pat. No. 8,192,954 which issued Jun. 5, 2012, which application is a continuation of U.S. patent application Ser. No. 12/609,847, filed Oct. 30, 2009 and now U.S. Pat. No. 7,888,035 which issued Feb. 15, 2011, which application claims the benefit of U.S. Provisional Application Nos. 61/109,742, filed Oct. 30, 2008; 61/112,571, filed Nov. 7, 2008; 61/114,045, filed Nov. 12, 2008; 61/114,058, filed Nov. 12, 2008; 61/114,065, filed Nov. 13, 2008; 61/151,183, filed Feb. 9, 2009; 61/278,049, filed Oct. 2, 2009; 61/250,454, filed Oct. 9, 2009, and 61/253,027 filed Oct. 19, 2009; and further which application is a continuation-in-part of U.S. patent application Ser. No. 13/009,285, filed Jan. 19, 2011 and now U.S. Pat. No. 8,211,653 which issued Jul. 3, 2012, which application is a continuation of U.S. patent application Ser. No. 12/658,452, filed Feb. 5, 2010 and now abandoned, which is a continuation of U.S. patent application Ser. No. 12/591,226 filed Nov. 12, 2009 and now U.S. Pat. No. 7,897,356 which issued Mar. 1, 2011, which claims the benefit of U.S. Provisional Application Nos. 61/114,045, filed Nov. 12, 2008; 61/114,058, filed Nov. 12, 2008; 61/114,065, filed Nov. 13, 2008; 61/151,183, filed Feb. 9, 2009; 61/278,049, filed Oct. 2, 2009; 61/250,454, filed Oct. 9, 2009; and 61/253,027 filed Oct. 19, 2009; each of which applications is incorporated herein by reference in its entirety.

### BACKGROUND

A critical need for disease detection, prognostic prediction, monitoring, and therapeutic decisions is improved assay sensitivity and specificity. At present, biomarkers (proteins, peptides, lipids, RNAs, DNA and modifications thereof for disease-associated molecular alterations) for conditions and diseases, such as cancer, rely almost exclusively on obtaining samples from tissue to identify the condition or disease. Methods to obtain these tissues of interest for analysis are often invasive, costly and pose complication risks for the patient. Furthermore, use of bodily fluids to isolate or detect biomarkers often significantly dilutes a biomarker resulting in readouts that lack requisite sensitivity. Additionally, most biomarkers are produced in low or moderate amounts in normal tissues other than the diseased tissue and thus this lack of specificity can also be problematic.

The identification of specific biomarkers, such as DNA, RNA and proteins can provide bio-signatures that are used for the diagnosis, prognosis, or theranosis of a condition or disease. Exosomes are a good source for assessing one or more biomarkers that are present in or on the surface of an exosome. Furthermore, identifying particular characteristics of an exosome (e.g., size, surface antigens, cell-of-origin) can itself provide a diagnostic, prognostic or theranostic readout.

The secretion of exosomes by cancerous cells, other diseased cells, or at certain times of a physiological process (e.g., pregnancy), can be leveraged to aid in diagnosis as well as individualized treatment decisions. Exosomes have been found in a number of body fluids, including blood plasma, breast milk, bronchoalveolar lavage fluid and urine. Exosomes also take part in the communication between cells, as transport vehicles for proteins, RNAs, DNAs, viruses, and prions.

The present inventions provide an improvement to prior art assays. Products and process are provided for improved assay sensitivity and specificity, allowing for disease detection, prognostic prediction, disease monitoring, disease staging, and therapeutic decision-making, as well as physiological state identification. Products and processes include cell-of-origin specific selection of exosomes and analysis of their protein composition, RNA composition, DNA composition, lipid profile, and relevant metabolic and/or epigenetic modifications of these analytes. Further provided are methods and composition for use of RNA biomarkers, e.g., microRNA biomarkers, which can be assessed in conjunction with exosomes or other biomarkers.

### SUMMARY

Disclosed herein are methods and compositions for characterizing a phenotype by analyzing an exosome. Characterizing a phenotype for a subject or individual may include, but is not limited to, the diagnosis of a disease or condition, the prognosis of a disease or condition, the determination of a disease stage or a condition stage, a drug efficacy, a physiological condition, organ distress or organ rejection, disease or condition progression, therapy-related association to a disease or condition, or a specific physiological or biological state.

The method can include determining a bio-signature of an exosome in a biological sample from a subject and characterizing a phenotype in said subject based on the bio-signature. Characterizing can also be based on determining the amount of exosomes in a biological sample. The characterization of the phenotype can be performed with at least 70, 80 or 90% sensitivity, specificity, or both.

The exosome can be isolated or concentrated prior to determining an exosomal bio-signature. The bio-signature can comprise an expression level, presence, absence, mutation, copy number variation, truncation, duplication, insertion, modification, sequence variation, or molecular association of a biomarker. The bio-signature can also comprise quantification of isolated exosomes, temporal evaluation of the variation in exosomal half-life, circulating exosomal half-life, exosomal metabolic half-life, or the activity of an exosome.

The exosome can be a cell-of-origin specific exosome. The exosome can be derived from a tumor or cancer cell. The cell-of-origin for an exosome can be a lung, pancreas, stomach, intestine, bladder, kidney, ovary, testis, skin, colorectal, breast, prostate, brain, esophagus, liver, placenta, or fetal cell.

One or more biomarkers of an exosome can be assessed for characterizing a phenotype. The biomarker can be a nucleic acid, peptide, protein, lipid, antigen, carbohydrate or proteoglycan, such as DNA or RNA. The RNA can be mRNA, miRNA, snoRNA, snRNA, rRNAs, tRNAs, siRNA, hnRNA, or shRNA. The biomarker can be an antigen selected from FIG. 1, or a biomarker selected from a table listed in FIG. 3-60. One or more biomarkers can be assessed and used to characterize a phenotype. The bio-signature can comprise one or more miRNAs selected from the group consisting of: miR-9, miR-629, miR-141, miR-671-3p, miR-491, miR-182, miR-125a-3p, miR-324-5p, miR-148b, and miR-222. The bio-signature can be used to characterize a phenotype, such as prostate cancer. Other biomarkers can be selected from the group consisting of: CD9, PSCA (prostate stem cell antigen), TNFR, CD63, MFG-E8, EpCam, Rab, CD81, STEAP, PCSA (prostate cell surface antigen), PSM (or PSMA, prostate specific membrane antigen), 5T4, CD59, CD66, CD24 and B7H3. Detecting a plurality of biomarkers can provide greater sensitivity or specificity as compared to detecting less than a plurality of biomarkers.

Methods of multiplexing, or multiplex analysis of, a plurality of exosomes are also provided. Multiplexing a plurality of exosomes can comprise applying said plurality of exosomes to a plurality of particles, wherein each particle of a subset of the plurality of particles is coupled to a different capture agent, capturing a subset of said plurality of exosomes; and, detecting one or more biomarkers of the captured exosomes. Multiplexing can also be performed using an array, wherein the capture agents are attached to an array instead of particles or beads.

Also provided herein are isolated exosomes. The isolated exosome can comprise any one or more biomarkers disclosed herein, such as a specific combination of biomarkers. Compositions comprising the one or more isolated exosomes are also provided. The composition can comprise a substantially enriched population of exosomes. The population of exosomes can be substantially homogeneous for one or more specific biomarkers, for a particular bio-signature, or derived from a specific cell type.

Detection systems, microfluidic devices, and kits for assessing one or more exosomes, such as for the isolation, separation, or detection of one or more exosomes, are also provided.

Still further provided herein are methods for characterizing a disease or condition by detecting or assessing an RNA or RNA pattern. Characterizing a condition can include diagnosing, prognosing, monitoring, selecting a treatment, or classifying a disease or condition, such as a cancer. The cancer can be an epithelial cancer, such as a breast, brain, pancreas, bone, liver, stomach, lung, colorectal, bladder, prostate or ovarian cancer. The RNA pattern can comprise detecting miRNAs, such as the expression level of miRNAs.

In some embodiments, the method includes characterizing a cancer in a subject comprising: determining a miRNA pattern in a biological sample of said subject, wherein the miRNA pattern comprises an expression level of each of a plurality of miRNAs in said sample. In some embodiments, characterizing is with increased sensitivity as compared to characterization by detecting an expression level of less than each of the plurality of miRNAs. The miRNAs can be selected from Table 2.

Also provided are methods of classifying a cancer, such as benign or malignant, and methods of determining if a solid tissue biopsy should be obtained after an initial analysis of a non-biopsy sample. The method can also further include selecting a therapy or treatment regimen based on the classification or results of the biopsy. Classifying a cancer or determining if a biopsy should be obtained can include determining the expression level of a miRNA, such as the copy number of the miRNA per microliter. The method can also include determining the expression level of PSA, such as the protein level, or a PCA3 score, which is the ratio between the PCA3 expression level and PSA expression level of a biological sample. The method can also include determining a product value to characterize a cancer. The product value can be determined by multiplying the expression level of a miRNA, such as miR-141, with the level of PSA. For example, the copy number per microliter of miRNA can be multiplied by the nanograms per microliters of PSA.

Also provided herein is a method of characterizing a cancer, such as prostate cancer, by determining the expression level of one or more miRNAs, such as miR-141, miR-629, miR-671-3p, miR-9, miR-491, miR-182, miR125a-3p, miR-324-5p, miR-148b, miR-222, or miR-370.

The RNA or RNA pattern can also be used in conjunction with other non-RNA biomarkers to characterize a cancer.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1 (a)-(g) represents a table which lists exemplary cancers by lineage, group comparisons of cells/tissue, and specific disease states and antigens specific to those cancers, group cell/tissue comparisons and specific disease states. Furthermore, the antigen can be a biomarker. The one or more biomarkers can be present or absent, underexpressed or overexpressed, mutated, or modified, such as epigentically modified or post-translationally modified.

FIG. 2 (a)-(f) represents a table which lists exemplary cancers by lineage, group comparisons of cells/tissue, and specific disease states and binding agents specific to those cancers, group cell/tissue comparisons and specific disease states.

FIG. 3 (a)-(b) represents a table which lists exemplary breast cancer biomarkers that can be derived and analyzed from exosomes specific to breast cancer to create a breast cancer specific exosome bio-signature. Furthermore, the one or more biomarkers can be present or absent, underexpressed or overexpressed, mutated, or modified, such as epigentically modified or post-translationally modified.

FIG. 4 (a)-(b) represents a table which lists exemplary ovarian cancer biomarkers that can be derived from and analyzed from exosomes specific to ovarian cancer to create an ovarian cancer specific exosome bio-signature. Furthermore, the one or more biomarkers can be present or absent, underexpressed or overexpressed, mutated, or modified, such as epigentically modified or post-translationally modified.

FIG. 5 represents a table which lists exemplary lung cancer biomarkers that can be derived from and analyzed from exosomes specific to lung cancer to create a lung cancer specific exosome bio-signature. Furthermore, the one or more biomarkers can be present or absent, underexpressed or overexpressed, mutated, or modified, such as epigentically modified or post-translationally modified.

FIG. 6 (a)-(d) represents a table which lists exemplary colon cancer biomarkers that can be derived from and analyzed from exosomes specific to colon cancer to create a colon cancer specific exosome bio-signature. Furthermore, the one or more biomarkers can be present or absent, underexpressed or overexpressed, mutated, or modified, such as epigentically modified or post-translationally modified.

FIG. 7 represents a table which lists exemplary biomarkers specific to an adenoma versus a hyperplastic polyp that can be derived and analyzed from exosomes specific to adenomas versus hyperplastic polyps. Furthermore, the one or more biomarkers can be present or absent, underexpressed or overexpressed, mutated, or modified, such as epigentically modified or post-translationally modified.

FIG. 8 is a table which lists exemplary biomarkers specific to inflammatory bowel disease (IBD) versus normal tissue that can be derived and analyzed from exosomes specific to inflammatory bowel disease versus normal tissue. Furthermore, the one or more biomarkers can be present or absent, underexpressed or overexpressed, mutated, or modified, such as epigentically modified or post-translationally modified.

FIG. 9(a)-(c) represents a table which lists exemplary biomarkers specific to an adenoma versus colorectal cancer (CRC) that can be derived and analyzed from exosomes specific to adenomas versus colorectal cancer. Furthermore, the one or more biomarkers can be present or absent, underexpressed or overexpressed, mutated, or modified, such as epigentically modified or post-translationally modified.

FIG. 10 represents a table which lists exemplary biomarkers specific to IBD versus CRC that can be derived and analyzed from exosomes specific to IBD versus CRC. Furthermore, the one or more biomarkers can be present or absent, underexpressed or overexpressed, mutated, or modified, such as epigentically modified or post-translationally modified.

FIG. 11 (a)-(b) represents a table which lists exemplary biomarkers specific to CRC Dukes B versus Dukes C-D that can be derived and analyzed from exosomes specific to CRC Dukes B versus Dukes C-D. Furthermore, the one or more biomarkers can be present or absent, underexpressed or overexpressed, mutated, or modified, such as epigentically modified or post-translationally modified.

FIG. 12(a)-(d) represents a table which lists exemplary biomarkers specific to an adenoma with low grade dysplasia versus an adenoma with high grade dysplasia that can be derived and analyzed from exosomes specific to an adenoma with low grade dysplasia versus an adenoma with high grade dysplasia. Furthermore, the one or more biomarkers can be present or absent, underexpressed or overexpressed, mutated, or modified, such as epigentically modified or post-translationally modified.

FIG. 13(a)-(b) represents a table which lists exemplary biomarkers specific to ulcerative colitis (UC) versus Crohn's Disease (CD) that can be derived and analyzed from exosomes specific to UC versus CD. Furthermore, the one or more biomarkers can be present or absent, underexpressed or overexpressed, mutated, or modified, such as epigentically modified or post-translationally modified.

FIG. 14 represents a table which lists exemplary biomarkers specific to a hyperplastic polyp versus normal tissue that can be derived and analyzed from exosomes specific to a hyperplastic polyp versus normal tissue. Furthermore, the one or more biomarkers can be present or absent, underexpressed or overexpressed, mutated, or modified, such as epigentically modified or post-translationally modified.

FIG. 15 is a table which lists exemplary biomarkers specific to an adenoma with low grade dysplasia versus normal tissue that can be derived and analyzed from exosomes specific to an adenoma with low grade dysplasia versus normal tissue. Furthermore, the one or more biomarkers can be present or absent, underexpressed or overexpressed, mutated, or modified, such as epigentically modified or post-translationally modified.

FIG. 16 is a table which lists exemplary biomarkers specific to an adenoma versus normal tissue that can be derived and analyzed from exosomes specific to an adenoma versus normal tissue. Furthermore, the one or more biomarkers can be present or absent, underexpressed or overexpressed, mutated, or modified, such as epigentically modified or post-translationally modified.

FIG. 17 represents a table which lists exemplary biomarkers specific to CRC versus normal tissue that can be derived and analyzed from exosomes specific to CRC versus normal tissue. Furthermore, the one or more biomarkers can be present or absent, underexpressed or overexpressed, mutated, or modified, such as epigentically modified or post-translationally modified.

FIG. 18 is a table which lists exemplary biomarkers specific to benign prostatic hyperplasia that can be derived from and analyzed from exosomes specific to benign prostatic hyperplasia. Furthermore, the one or more biomarkers can be present or absent, underexpressed or overexpressed, mutated, or modified, such as epigentically modified or post-translationally modified.

FIG. 19(a)-(c) represents a table which lists exemplary prostate cancer biomarkers that can be derived from and analyzed from exosomes specific to prostate cancer to create a prostate cancer specific exosome bio-signature. Furthermore, the one or more biomarkers can be present or absent, underexpressed or overexpressed, mutated, or modified, such as epigentically modified or post-translationally modified.

FIG. 20(a)-(c) represents a table which lists exemplary melanoma biomarkers that can be derived from and analyzed from exosomes specific to melanoma to create a melanoma specific exosome bio-signature. Furthermore, the one or more biomarkers can be present or absent, underexpressed or overexpressed, mutated, or modified, such as epigentically modified or post-translationally modified.

FIG. 21(a)-(b) represents a table which lists exemplary pancreatic cancer biomarkers that can be derived from and analyzed from exosomes specific to pancreatic cancer to create a pancreatic cancer specific exosome bio-signature. Furthermore, the one or more biomarkers can be present or absent, underexpressed or overexpressed, mutated, or modified, such as epigentically modified or post-translationally modified.

FIG. 22 is a table which lists exemplary biomarkers specific to brain cancer that can be derived from and analyzed from exosomes specific to brain cancer to create a brain cancer specific exosome bio-signature. Furthermore, the one or more biomarkers can be present or absent, underexpressed or overexpressed, mutated, or modified, such as epigentically modified or post-translationally modified.

FIG. 23(a)-(b) represents a table which lists exemplary psoriasis biomarkers that can be derived from and analyzed from exosomes specific to psoriasis to create a psoriasis specific exosome bio-signature. Furthermore, the one or more biomarkers can be present or absent, underexpressed or overexpressed, mutated, or modified, such as epigentically modified or post-translationally modified.

FIG. 24(a)-(c) represents a table which lists exemplary cardiovascular disease biomarkers that can be derived from and analyzed from exosomes specific to cardiovascular disease to create a cardiovascular disease specific exosome bio-signature. Furthermore, the one or more biomarkers can be present or absent, underexpressed or overexpressed, mutated, or modified, such as epigentically modified or post-translationally modified.

FIG. 25 is a table which lists exemplary biomarkers specific to hematological malignancies that can be derived from and analyzed from exosomes specific to hematological malignancies to create a specific exosome bio-signature for hematological malignancies. Furthermore, the one or more biomarkers can be present or absent, underexpressed or overexpressed, mutated, or modified, such as epigentically modified or post-translationally modified.

FIG. 26(a)-(b) represents a table which lists exemplary biomarkers specific to B-Cell Chronic Lymphocytic Leukemias that can be derived from and analyzed from exosomes specific to B-Cell Chronic Lymphocytic Leukemias to create a specific exosome bio-signature for B-Cell Chronic Lymphocytic Leukemias. Furthermore, the one or more biomarkers can be present or absent, underexpressed or overexpressed, mutated, or modified, such as epigentically modified or post-translationally modified.

FIG. 27 is a table which lists exemplary biomarkers specific to B-Cell Lymphoma and B-Cell Lymphoma-DLBCL that can be derived from and analyzed from exosomes specific to B-Cell Lymphoma and B-Cell Lymphoma-DLBCL. Furthermore, the one or more biomarkers can be present or absent, underexpressed or overexpressed, mutated, or modified, such as epigentically modified or post-translationally modified.

FIG. 28 represents a table which lists exemplary biomarkers specific to B-Cell Lymphoma-DLBCL-germinal center-like and B-Cell Lymphoma-DLBCL-activated B-cell-like and B-cell lymphoma-DLBCL that can be derived from and analyzed from exosomes specific to B-Cell Lymphoma-DLBCL-germinal center-like and B-Cell Lymphoma-DLBCL-activated B-cell-like and B-cell lymphoma-DLBCL. Furthermore, the one or more biomarkers can be present or absent, underexpressed or overexpressed, mutated, or modified, such as epigentically modified or post-translationally modified.

FIG. 29 represents a table which lists exemplary Burkitt's lymphoma biomarkers that can be derived from and analyzed from exosomes specific to Burkitt's lymphoma to create a Burkitt's lymphoma specific exosome bio-signature. Furthermore, the one or more biomarkers can be present or absent, underexpressed or overexpressed, mutated, or modified, such as epigentically modified or post-translationally modified.

FIG. 30(a)-(b) represents a table which lists exemplary hepatocellular carcinoma biomarkers that can be derived from and analyzed from exosomes specific to hepatocellular carcinoma to create a specific exosome bio-signature for hepatocellular carcinoma. Furthermore, the one or more biomarkers can be present or absent, underexpressed or overexpressed, mutated, or modified, such as epigentically modified or post-translationally modified.

FIG. 31 is a table which lists exemplary biomarkers for cervical cancer that can be derived from and analyzed from exosomes specific to cervical cancer. Furthermore, the one or more biomarkers can be present or absent, underexpressed or overexpressed, mutated, or modified, such as epigentically modified or post-translationally modified.

FIG. 32 represents a table which lists exemplary biomarkers for endometrial cancer that can be derived from and analyzed from exosomes specific to endometrial cancer to create a specific exosome bio-signature for endometrial cancer. Furthermore, the one or more biomarkers can be present or absent, underexpressed or overexpressed, mutated, or modified, such as epigentically modified or post-translationally modified.

FIG. 33(a)-(b) represents a table which lists exemplary biomarkers for head and neck cancer that can be derived from and analyzed from exosomes specific to head and neck cancer to create a specific exosome bio-signature for head and neck cancer. Furthermore, the one or more biomarkers can be present or absent, underexpressed or overexpressed, mutated, or modified, such as epigentically modified or post-translationally modified.

FIG. 34 represents a table which lists exemplary biomarkers for inflammatory bowel disease (IBD) that can be derived from and analyzed from exosomes specific to IBD to create a specific exosome bio-signature for IBD. Furthermore, the one or more biomarkers can be present or absent, underexpressed or overexpressed, mutated, or modified, such as epigentically modified or post-translationally modified.

FIG. 35 is a table which lists exemplary biomarkers for diabetes that can be derived from and analyzed from exosomes specific to diabetes to create a specific exosome bio-signature for diabetes. Furthermore, the one or more biomarkers can be present or absent, underexpressed or overexpressed, mutated, or modified, such as epigentically modified or post-translationally modified.

FIG. 36 is a table which lists exemplary biomarkers for Barrett's Esophagus that can be derived from and analyzed from exosomes specific to Barrett's Esophagus to create a specific exosome bio-signature for Barrett's Esophagus. Furthermore, the one or more biomarkers can be present or absent, underexpressed or overexpressed, mutated, or modified, such as epigentically modified or post-translationally modified.

FIG. 37 is a table which lists exemplary biomarkers for fibromyalgia that can be derived from and analyzed from exosomes specific to fibromyalgia. Furthermore, the one or more biomarkers can be present or absent, underexpressed or overexpressed, mutated, or modified, such as epigentically modified or post-translationally modified.

FIG. 38 represents a table which lists exemplary biomarkers for stroke that can be derived from and analyzed from exosomes specific to stroke to create a specific exosome bio-signature for stroke. Furthermore, the one or more biomarkers can be present or absent, underexpressed or overexpressed, mutated, or modified, such as epigentically modified or post-translationally modified.

FIG. 39 is a table which lists exemplary biomarkers for Multiple Sclerosis (MS) that can be derived from and analyzed from exosomes specific to MS to create a specific exosome bio-signature for MS. Furthermore, the one or more biomarkers can be present or absent, underexpressed or overexpressed, mutated, or modified, such as epigentically modified or post-translationally modified.

FIG. 40(a)-(b) represents a table which lists exemplary biomarkers for Parkinson's Disease that can be derived from and analyzed from exosomes specific to Parkinson's Disease to create a specific exosome bio-signature for Parkinson's Disease. Furthermore, the one or more biomarkers can be present or absent, underexpressed or overexpressed, mutated, or modified, such as epigentically modified or post-translationally modified.

FIG. 41 represents a table which lists exemplary biomarkers for Rheumatic Disease that can be derived from and analyzed from exosomes specific to Rheumatic Disease to create a specific exosome bio-signature for Rheumatic Disease. Furthermore, the one or more biomarkers can be present or absent, underexpressed or overexpressed, mutated, or modified, such as epigentically modified or post-translationally modified.

FIG. 42(a)-(b) represents a table which lists exemplary biomarkers for Alzheimers Disease that can be derived from and analyzed from exosomes specific to Alzheimers Disease to create a specific exosome bio-signature for Alzheimers Disease. Furthermore, the one or more biomarkers can be present or absent, underexpressed or overexpressed, mutated, or modified, such as epigentically modified or post-translationally modified.

FIG. 43 is a table which lists exemplary biomarkers for Prion Diseases that can be derived from and analyzed from exosomes specific to Prion Diseases to create a specific exosome bio-signature for Prion Diseases. Furthermore, the one or more biomarkers can be present or absent, underexpressed or overexpressed, mutated, or modified, such as epigentically modified or post-translationally modified.

FIG. 44 represents a table which lists exemplary biomarkers for sepsis that can be derived from and analyzed from exosomes specific to sepsis to create a specific exosome bio-signature for sepsis. Furthermore, the one or more biomarkers can be present or absent, underexpressed or overexpressed, mutated, or modified, such as epigentically modified or post-translationally modified.

FIG. 45 is a table which lists exemplary biomarkers for chronic neuropathic pain that can be derived from and analyzed from exosomes specific to chronic neuropathic pain. Furthermore, the one or more biomarkers can be present or absent, underexpressed or overexpressed, mutated, or modified, such as epigentically modified or post-translationally modified.

FIG. 46 is a table which lists exemplary biomarkers for peripheral neuropathic pain that can be derived from and analyzed from exosomes specific to peripheral neuropathic pain. Furthermore, the one or more biomarkers can be present or absent, underexpressed or overexpressed, mutated, or modified, such as epigentically modified or post-translationally modified.

FIG. 47 represents a table which lists exemplary biomarkers for Schizophrenia that can be derived from and analyzed from exosomes specific to Schizophrenia to create a specific exosome bio-signature for Schizophrenia. Furthermore, the one or more biomarkers can be present or absent, underexpressed or overexpressed, mutated, or modified, such as epigentically modified or post-translationally modified.

FIG. 48 is a table which lists exemplary biomarkers for bipolar disorder or disease that can be derived from and analyzed from exosomes specific to bipolar disorder to create a specific exosome bio-signature for bipolar disorder. Furthermore, the one or more biomarkers can be present or absent, underexpressed or overexpressed, mutated, or modified, such as epigentically modified or post-translationally modified.

FIG. 49 is a table which lists exemplary biomarkers for depression that can be derived from and analyzed from exosomes specific to depression to create a specific exosome bio-signature for depression. Furthermore, the one or more biomarkers can be present or absent, underexpressed or overexpressed, mutated, or modified, such as epigentically modified or post-translationally modified.

FIG. 50 is a table which lists exemplary biomarkers for gastrointestinal stromal tumor (GIST) that can be derived from and analyzed from exosomes specific to GIST to create a specific exosome bio-signature for GIST. Furthermore, the one or more biomarkers can be present or absent, underexpressed or overexpressed, mutated, or modified, such as epigentically modified or post-translationally modified.

FIG. 51(a)-(b) represent sa table which lists exemplary biomarkers for renal cell carcinoma (RCC) that can be derived from and analyzed from exosomes specific to RCC to create a specific exosome bio-signature for RCC. Furthermore, the one or more biomarkers can be present or absent, underexpressed or overexpressed, mutated, or modified, such as epigentically modified or post-translationally modified.

FIG. 52 is a table which lists exemplary biomarkers for cirrhosis that can be derived from and analyzed from exosomes specific to cirrhosis to create a specific exosome bio-signature for cirrhosis. Furthermore, the one or more biomarkers can be present or absent, underexpressed or overexpressed, mutated, or modified, such as epigentically modified or post-translationally modified.

FIG. 53 is a table which lists exemplary biomarkers for esophageal cancer that can be derived from and analyzed from exosomes specific to esophageal cancer to create a specific exosome bio-signature for esophageal cancer. Furthermore, the one or more biomarkers can be present or absent, underexpressed or overexpressed, mutated, or modified, such as epigentically modified or post-translationally modified.

FIG. 54 is a table which lists exemplary biomarkers for gastric cancer that can be derived from and analyzed from exosomes specific to gastric cancer to create a specific exosome bio-signature for gastric cancer. Furthermore, the one or more biomarkers can be present or absent, underexpressed or overexpressed, mutated, or modified, such as epigentically modified or post-translationally modified.

FIG. 55 is a table which lists exemplary biomarkers for autism that can be derived from and analyzed from exosomes specific to autism to create a specific exosome bio-signature for autism. Furthermore, the one or more biomarkers can be present or absent, underexpressed or overexpressed, mutated, or modified, such as epigentically modified or post-translationally modified.

FIG. 56 is a table which lists exemplary biomarkers for organ rejection that can be derived from and analyzed from exosomes specific to organ rejection to create a specific exosome bio-signature for organ rejection. Furthermore, the one or more biomarkers can be present or absent, underexpressed or overexpressed, mutated, or modified, such as epigentically modified or post-translationally modified.

FIG. 57 is a table which lists exemplary biomarkers for methicillin-resistant staphylococcus aureus that can be derived from and analyzed from exosomes specific to methicillin-resistant staphylococcus aureus to create a specific exosome bio-signature for methicillin-resistant staphylococcus aureus. Furthermore, the one or more biomarkers can be present or absent, underexpressed or overexpressed, mutated, or modified, such as epigentically modified or post-translationally modified.

FIG. 58 is a table which lists exemplary biomarkers for vulnerable plaque that can be derived from and analyzed from exosomes specific to vulnerable plaque to create a specific exosome bio-signature for vulnerable plaque. Furthermore, the one or more biomarkers can be present or absent, underexpressed or overexpressed, mutated, or modified, such as epigentically modified or post-translationally modified.

FIG. 59(a)-(i) is a table which lists exemplary gene fusions that can be derived from, or analyzed from exosomes. The gene fusion can be biomarker, and can be present or absent, underexpressed or overexpressed, or modified, such as epigentically modified or post-translationally modified.

FIG. 60(a)-(b) is a table of genes and their associated miRNAs, of which the gene, such as the mRNA of the gene, their associated miRNAs, or any combination thereof, can be used as one or more biomarkers that can be analyzed from exosomes. Furthermore, the one or more biomarkers can be present or absent, underexpressed or overexpressed, mutated, or modified.

FIG. 61 is a flow chart of an exemplary method disclosed herein.

FIG. 62 illustrates a computer system that can be used in some exemplary embodiments of the invention.

FIG. 63 illustrates results obtained from screening for proteins on exosomes, which can be used a biomarkers for the exosomes and antibodies to the proteins can be used as binding agents. Examples of the proteins identified include Bcl-XL, ERCC1, Keratin 15, CD81/TAPA-1, CD9, Epithelial Specific Antigen (ESA), and Mast Cell Chymase. The one or more biomarkers can be present or absent, underexpressed or overexpressed, mutated, or modified.

FIGS. 64A-C illustrate a planar substrate (A) and particle based (B) method of isolating exosomes. (A) is a schematic of a planar surfact and (B) is a schematic of a bead coated with a capture antibody, which captures exosomes expressing that protein. In this schematic, the capture antibody is for an exosomal protein that is not specific for exosomes derived from cancer cells (“cancer exosome”). The detection antibody binds to the captured exosome and fluoresces a signal. The detection antibody in this example detects an antigen that is associated with cancer exosomes. (C) is an example of a screening scheme that can be performed by multiplexing using the planar substrate as shown in (A) or particle substrate as shown in (B).

FIGS. 65A-C depict scanning electron micrographs (SEMs) of EpCam conjugated beads that have been incubated with VCaP exosomes. (A) A glass slide was coated with poly-L-lysine and incubated with the bead solution. After attachment, the beads were (i) fixed sequentially with glutaraldehyde and osmium tetroxide, 30 min per fix step with a few washes in between; (ii) gradually dehydrated in acetone, 20% increments, about 5-7 min per step; (iii) critical-point dried; and (iv) sputter-coated with gold. (B) depicts a higher magnification of exosomes on an EpCam coated bead as in (A). (C) depicts exosomes isolated by ultracentrifugation and adhered to a poly-L-lysine coated glass slide and fixed and stained as in (A).

FIG. 66 is a schematic of an exosome protein expression patterns. Different proteins are typically not distributed evenly or uniformly on exosome shell. Exosome-specific proteins are typically more common, while cancer-specific proteins are less common Exosome capture can be more easily accomplished using a more common, less cancer-specific protein, and cancer-specific proteins used in the detection phase.

FIGS. 67A-C illustrate the method of depicting the results using the bead based method of detecting exosomes from a subject. FIGS. 67A-B show graphs of the bead enumeration and signal intensity for an individual normal patient (without cancer) (A) or for an individual with cancer (B) using a screening scheme as depicted in FIG. 64C, where ˜100 capture beads are used for each capture/detection combination assay per patient. For a given patient, the output shows number of beads detected vs. intensity of signal. The number of beads captured at a given intensity is an indication of how frequently an exosome expresses the detection protein at that intensity. The more intense the signal for a given bead, the greater the expression of the detection protein. (C) is a normalized graph obtained by combining normal patients into one curve and cancer patients into another, and using bio-statistical analysis to differentiate the curves. Data from each individual is normalized to account for variation in the number of beads read by the detection machine, added together, and then normalized again to account for the different number of samples in each population.

FIGS. 68A-R illustrate prostate cancer bio-signatures. (A)-(D) are histograms of intensity values collected from a multiplexing experiment using the Luminex platform, where beads were functionalized with CD63 antibody, incubated with exosomes purified from patient plasma, and then labeled with a phycoerythrin (PE) conjugated EpCam antibody (A), CD81 antibody (B), CD63 antibody (C) or CD9 antibody (D). The grey shaded bars represent the population from 12 normal subjects and the white shaded bars are from 7 stage 3 prostate cancer patients. (E)-(H) are normalized graphs for each of the histograms shown in (A)-(D), respectively, as described for FIGS. 67A-C. The distributions are of a Gaussian fit to intensity values from the Luminex results of (A)-(D) for both prostate patient samples and normal samples. (I) is an example of the prostate bio-signatures shown in (C), the CD63 versus CD63 bio-signature where CD63 is used as the detector and capture antibody. (J)-(L) show the results of flow cytometry on three prostate cancer cell lines (VCaP (J), LNcap (K), and 22RV1 (L)). Points above the horizontal line indicate beads that captured exosomes with CD63 that contain B7H3. Beads to the right of the vertical line indicate beads that have captured exosomes with CD63 that have PSMA. Those beads that are above and to the right of the lines have all three antigens. CD63 is a surface protein that is associated with exosomes, PSMA is surface protein that is associated with prostate cells, and B7H3 is a surface protein that is associated with aggressive cancers (specifically prostate, ovarian, and non-small-cell lung). The combination of all three antigens together identifies exosomes that are from cancer prostate cells. The majority of CD63 expressing prostate cancer exosomes also have prostate-specific membrane antigen, PSMA, and B7H3 (implicated in regulation of tumor cell migration and invasion and an indicator of aggressive cancer as well as clinical outcome). (M)-(R) show prostate cancer exosome topography. (M)-(O) show the results of capturing and labeling with CD63, CD9, and CD81 in various combinations. Almost all points are in the upper right quadrant indicating that these three markers are highly coupled. If an exosome has one of them, it typically has all three. (P)-(R) depict the results of capturing cell line exosomes with B7H3 and labeling with CD63 and PSMA. Both VCaP (P) and 22RV1 (R) show that most exosomes captured with B7H3 also have CD63, and that there are two populations, those with PSMA and those without. The presence of B7H3 may be an indication of how aggressive the cancer is, as LNcap (Q) does not have a high amount of B7H3 containing exosomes (not many spots with CD63). LnCap is an earlier stage prostate cancer analogue cell line.

FIGS. 69A-J illustrate colon cancer bio-signatures. (A)-(D) show histograms of intensity values collected from various multiplexing experiments using the Luminex platform, where beads were functionalized with a capture antibody, incubated with exosomes purified form patient plasma, and then labeled with a detector antibody. The grey shaded bars represent the population from normals and the white bars are from colon cancer patients. (E)-(H) show normalized graphs for each of the histograms shown in (A)-(D), respectively. (I)-(J) show histograms of intensity values collected from a Luminex experiment where beads where functionalized with CD66 antibody (the capture antibody), incubated with exosomes purified from patient plasma, and then labeled with a PE conjugated EpCam antibody (the detector antibody). The grey population is from 6 normals and the white is from 21 colon cancer patients. (J) shows data from each individual normalized to account for variation in the number of beads read by the Luminex machine, added together, and then normalized again to account for the different number of samples in each population.

FIGS. 70A-F illustrates multiple detectors can increase the signal of exosome detection. (A)-(D) Median intensity values are plotted as a function of purified exosome concentration from the VCaP cell line when labeled with a variety of prostate specific PE conjugated antibodies. Exosomes captured with EpCam ((A) and (C)) or PCSA ((B) and (D)) and the various proteins detected by the detector antibody are listed to the right of each graph. In both cases the combination of CD9 and CD63 gives the best increase in signal over background ((C) and (D) depicting percent increase). The combination of CD9 and CD63 gave about 200% percent increase over background. (E)-(F) further illustrate prostate cancer/prostate exosome-specific marker multiplexing improves detection of prostate cancer cell derived exosomes. Median intensity values are plotted as a function of purified exosome concentration from the VCaP cell line when labeled with a variety of prostate specific PE conjugated antibodies. Exosomes captured with PCSA (E) and exosomes captured with EpCam (F) are depicted. In both cases the combination of B7H3 and PSMA gives the best increase in signal over background.

FIGS. 71A-F illustrate a colon cancer bio-signature for colon cancer by stage, using CD63 detector and CD63 capture. The histograms of intensities from exosomes captured with CD63 coated beads and labeled with CD63 conjugated PE. There are 6 patients in the control group (A), 4 in stage I (B), 5 in stage II (C), 8 in stage III (D), and 4 stage IV (E). Data from each individual was normalized to account for variation in the number of beads read by the Luminex machine, added together, and then normalized again to account for the different number of samples in each population (F).

FIGS. 72A-F illustrate colon cancer bio-signature for colon cancer by stage, using EpCam detector and CD9 capture. The histograms of intensities are from exosomes captured with CD9 coated beads and labeled with EpCam. There are patients in the (A) control group, (B) stage I, (C) stage II, (D) stage III, and (E) stage IV. Data from each individual was normalized to account for variation in the number of beads read by the Luminex machine, added together, and then normalized again to account for the different number of samples in each population (F).

FIGS. 73A-E illustrate (A) the sensitivity and specificity, and the confidence level, for detecting prostate cancer using antibodies to the listed proteins listed as the detector and capture antibodies. CD63, CD9, and CD81 are general exosome markers and EpCam is a cancer marker. The individual results are depicted in (B) for EpCam versus CD63, with 99% confidence, 100% (n=8) cancer patient samples were different from the Generalized Normal Distribution and with 99% confidence, 77% (n=10) normal patient samples were not different from the Generalized Normal Distribution; (C) for CD81 versus CD63, with 99% confidence, 90% (n=5) cancer patient samples were different from the Generalized Normal Distribution; with 99% confidence, 77% (n=10) normal patient samples were not different from the Generalized Normal Distribution; (D) for CD63 versus CD63, with 99% confidence, 60% (n=5) cancer patient samples were different from the Generalized Normal Distribution; with 99% confidence, 80% (n=10) normal patient samples were not different from the Generalized Normal Distribution; (E) for CD9 versus CD63, with 99% confidence, 90% (n=5) cancer patient samples were different from the Generalized Normal Distribution; with 99% confidence, 77% (n=10) normal patient samples were not different from the Generalized Normal Distribution.

FIGS. 74A-F illustrate (A) the sensitivity and the confidence level for detecting colon cancer using antibodies to the listed proteins listed as the detector and capture antibodies. CD63, CD9 are general exosome markers, EpCam is a cancer marker, and CD66 is a colon marker. The individual results are depicted in (B) for EpCam versus CD63, with 99% confidence, 95% (n=20) cancer patient samples were different from the Generalized Normal Distribution; with 99% confidence, 100% (n=6) normal patient samples were not different from the Generalized Normal Distribution; (C) for EpCam versus CD9, with 99% confidence, 90% (n=20) cancer patient samples were different from the Generalized Normal Distribution; with 99% confidence, 77% (n=6) normal patient samples were not different from the Generalized Normal Distribution; (D) for CD63 versus CD63, with 99% confidence, 60% (n=20) cancer patient samples were different from the Generalized Normal Distribution; with 99% confidence, 80% (n=6) normal patient samples were not different from the Generalized Normal Distribution; (E) for CD9 versus CD63, with 99% confidence, 90% (n=20) cancer patient samples were different from the Generalized Normal Distribution; with 99% confidence, 77% (n=6) normal patient samples were not different from the Generalized Normal Distribution; (F) for CD66 versus CD9, with 99% confidence, 90% (n=20) cancer patient samples were different from the Generalized Normal Distribution; with 99% confidence, 77% (n=6) normal patient samples were not different from the Generalized Normal Distribution.

FIGS. 75A-C illustrates the capture of prostate cancer cells-derived exosomes from plasma with EpCam by assessing TMPRSS2-ERG expression. (A) Graduated amounts of VCAP purified exosomes were spiked into normal plasma. Exosomes were isolated using Dynal beads with either EPCAM antibody or its isotype control. RNA from the exosomes was isolated and the expression of the TMPRSS2:ERG fusion transcript was measured using qRT-PCR. (B) VCaP purified exosomes were spiked into normal plasma and then incubated with Dynal magnetic beads coated with either the EpCam or isotype control antibody. RNA was isolated directly from the Dynal beads. Equal volumes of RNA from each sample were used for RT-PCR and subsequent Taqman assays. (C) Cycle threshold (CT) differences of the SPINK1 and GAPDH transcripts between 22RV1 exosomes captured with EpCam and IgG2 isotype negative control beads. Higher CT values indicate lower transcript expression.

FIGS. 76A-B illustrate the top ten differentially expressed microRNAs between VCaP prostate cancer cell derived exosomes and normal plasma exosomes. VCAP cell line exosomes and exosomes from normal plasma were isolated via ultracentrifugation followed by RNA isolation. MicroRNAs were profiled using qRT-PCR analysis. Prostate cancer cell line derived exosomes have higher levels (lower CT values) of the indicated microRNAs as depicted in the bar graph (A) and table (B).

FIG. 77 depicts a bar graph of miR-21 expression with CD9 bead capture. 1 ml of plasma from prostate cancer patients, 250 ng/ml of LNCaP, or normal purified exosomes was incubated with CD9 coated Dynal beads. The RNA was isolated from the beads and the bead supernatant. One sample (#6) was also uncaptured for comparison. MiR-21 expression was measured with qRT-PCR and the mean CT values for each sample compared. CD9 capture improves the detection of miR-21 in prostate cancer samples.

FIG. 78 depicts a bar graph of miR-141 expression with CD9 bead capture. The experiment was performed as in FIG. 77, with miR-141 expression measured with qRT-PCR instead of miR-21.

FIG. 79 depicts a table of the sensitivity and specificity for different prostate signatures. “Exosome” lists the threshold value or reference value of exosome levels, “Prostate” lists the threshold value or reference value used for prostate exosomes, “Cancer-1,” “Cancer-2,” and “Cancer-3” lists the threshold values or reference values for the three different bio-signatures for prostate cancer, the “QC-1” and “QC-2” columns list the threshold values or reference values for quality control, or reliability, and the last four columns list the specificities and sensitivities for benign prostate hyperplasia (BPH).

FIG. 80 illustrates expression profiles for 6 prostate cancer samples. This figure shows the expression profile from the Agilent gene chip analysis on the 6 prostate cancer samples with the gene names listed on the right. Dark coloring or shading indicates high expression levels.

FIG. 81 is a graph illustrating the frequency of a miRNA (listed along the x-axis), by analyzing the most frequently over-expressed genes in the prostate cancer samples in a database by both immunohistochemistry (IHC) and gene expression profiling on the Agilent 44K chip, searching a publicly available miRNA database for microRNAs known to be related to those genes (for example, such as world wide web at microRNA.org), and ranking the miRNAs by frequency observed.

### DETAILED DESCRIPTION

Disclosed herein are products and processes for characterizing a phenotype of an individual by analyzing exosomes. A phenotype can be any observable characteristic or trait of a subject, such as a disease or condition, a disease stage or condition stage, susceptibility to a disease or condition, prognosis of a disease stage or condition, a physiological state, or response to therapeutics. A phenotype can result from a subject's gene expression as well as the influence of environmental factors and the interactions between the two, as well as from epigenetic modifications to nucleic acid sequences

A phenotype in a subject can be characterized by obtaining a biological sample from said subject and analyzing one or more exosomes from the sample. For example, characterizing a phenotype for a subject or individual may include detecting a disease or condition (including pre-symptomatic early stage detecting), determining the prognosis, diagnosis, or theranosis of a disease or condition, or determining the stage or progression of a disease or condition. Characterizing a phenotype can also include identifying appropriate treatments or treatment efficacy for specific diseases, conditions, disease stages and condition stages, predictions and likelihood analysis of disease progression, particularly disease recurrence, metastatic spread or disease relapse. A phenotype can also be a clinically distinct type or subtype of a condition or disease, such as a cancer or tumor. Phenotype determination can also be a determination of a physiological condition, or an assessment of organ distress or organ rejection, such as post-transplantation. The products and processes described herein allow assessment of a subject on an individual basis, which can provide benefits of more efficient and economical decisions in treatment.

The phenotype can be a disease or condition such as listed in Table 1. For example, the phenotype can be a tumor, neoplasm, or cancer. A cancer detected or assessed by products or processes described herein includes, but is not limited to, breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer can be CRC Dukes B or Dukes C-D. The hematological malignancy can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, and Burkitt's lymphoma. The phenotype may also be a premalignant condition, such as Barrett's Esophagus.

The phenotype can also be an inflammatory disease, immune disease, or autoimmune disease. For example, the disease may be inflammatory bowel disease (IBD), Crohn's disease (CD), ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis.

The phenotype can also be a cardiovascular disease, such as atherosclerosis, congestive heart failure, vulnerable plaque, stroke, or ischemia. The cardiovascular disease or condition can be high blood pressure, stenosis, vessel occlusion or a thrombotic event.

The phenotype can also be a neurological disease, such as Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neurospsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The phenotype may also be a condition such as fibromyalgia, chronic neuropathic pain, or peripheral neuropathic pain.

The phenotype may also be an infectious disease, such as a bacterial, viral or yeast infection. For example, the disease or condition may be Whipple's Disease, Prion Disease, cirrhosis, methicillin-resistant staphylococcus aureus, HIV, hepatitis, syphilis, meningitis, malaria, tuberculosis, or influenza. Viral proteins, such as HIV or HCV-like particles can be assessed in an exosome, to characterize a viral condition.

The phenotype can also be a perinatal or pregnancy related condition (e.g. preeclampsia or preterm birth), metabolic disease or condition, such as a metabolic disease or condition associated with iron metabolism. For example, hepcidin can be assayed in an exosome to characterize an iron deficiency. The metabolic disease or condition can also be diabetes, inflammation, or a perinatal condition.

Subject

One or more phenotypes of a subject can be determined by analyzing exosomes in a biological sample obtained from the subject. A subject or patient can include, but is not limited to, mammals such as bovine, avian, canine, equine, feline, ovine, porcine, or primate animals (including humans and non-human primates). A subject may also include mammals of importance due to being endangered, such as Siberian tigers; or economic importance, such as animals raised on farms for consumption by humans, or animals of social importance to humans such as animals kept as pets or in zoos. Examples of such animals include but are not limited to: carnivores such as cats and dogs; swine including pigs, hogs and wild boars; ruminants or ungulates such as cattle, oxen, sheep, giraffes, deer, goats, bison, camels or horses. Also included are birds that are endangered or kept in zoos, as well as fowl and more particularly domesticated fowl, i.e. poultry, such as turkeys and chickens, ducks, geese, guinea fowl. Also included are domesticated swine and horses (including race horses). In addition, any animal species connected to commercial activities are also included such as those animals connected to agriculture and aquaculture and other activities in which disease monitoring, diagnosis, and therapy selection are routine practice in husbandry for economic productivity and/or safety of the food chain.

The subject can have a pre-existing disease or condition, such as cancer. Alternatively, the subject may not have any known pre-existing condition. The subject may also be non-responsive to an existing or past treatment, such as a treatment for cancer.

Samples

The biological sample obtained from the subject may be any bodily fluid. For example, the biological sample can be peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates or other lavage fluids. A biological sample may also include the blastocyl cavity, umbilical cord blood, or maternal circulation which may be of fetal or maternal origin. The biological sample may also be a tissue sample or biopsy, from which exosomes may be obtained. For example, if the sample is a solid sample, cells from the sample can be cultured and exosome product induced (see for example, Example 1).

Table 1 provides a list of examples of diseases, conditions, or biological states and a corresponding list of biological samples from which exosomes may be analyzed.

TABLE 1
Examples of Biological Samples for Exosome Analysis for Various Diseases, Conditions, or
Biological States

| Disease, Condition or Biological State | Biological Samples |
|---|---|
| Cancers/neoplasms affecting the following tissue | Blood, serum, cerebrospinal fluid (CSF), urine, sputum, |
| types/bodily systems: breast, lung, ovarian, colon, rectal, | ascites, synovial fluid, semen, nipple aspirates, saliva, |
| prostate, pancreatic, brain, bone, connective tissue, glands, | bronchoalveolar lavage fluid, tears, oropharyngeal washes, |
| skin, lymph, nervous system, endocrine, germ cell, | feces, peritoneal fluids, pleural effusion, sweat, tears, |
| genitourinary, hematologic/blood, bone marrow, muscle, | aqueous humor, pericardial fluid, lymph, chyme, chyle, |
| eye, esophageal, fat tissue, thyroid, pituitary, spinal cord, | bile, stool water, amniotic fluid, breast milk, pancreatic |
| bile duct, heart, gall bladder, bladder, testes, cervical, | juice, cerumen, Cowper's fluid or pre-ejaculatory fluid, |
| endometrial, renal, ovarian, digestive/gastrointestinal, | female ejaculate, interstitial fluid, menses, mucus, pus, |
| stomach, head and neck, liver, leukemia, | sebum, vaginal lubrication, vomit |
| respiratory/thorasic, cancers of unknown primary | |
| Neurodegenerative/neurological disorders: Parkinson's | Blood, serum, CSF, urine |
| disease, Alzheimer's Disease and multiple sclerosis, | |
| Schizophrenia, and bipolar disorder, spasticity disorders, | |
| epilepsy | |
| Cardiovascular Disease: atherosclerosis, cardiomyopathy, | Blood, serum, CSF, urine |
| endocarditis, vunerable plaques, infection | |
| Stroke: ischemic, intracerebral hemorrhage, subarachnoid | Blood, serum, CSF, urine |
| hemorrhage, transient ischemic attacks (TIA) | |
| Pain disorders: peripheral neuropathic pain and chronic | Blood, serum, CSF, urine |
| neuropathic pain, and fibromyalgia, | |
| Autoimmune disease: systemic and localized diseases, | Blood, serum, CSF, urine, synovial fluid |
| rheumatic disease, Lupus, Sjogren's syndrome | |
| Digestive system abnormalities: Barrett's esophagus, | Blood, serum, CSF, urine |
| irritable bowel syndrome, ulcerative colitis, Crohn's | |
| disease, Diverticulosis and Diverticulitis, Celiac Disease | |
| Endocrine disorders: diabetes mellitus, various forms of | Blood, serum, CSF, urine |
| Thyroiditis,, adrenal disorders, pituitary disorders | |
| Diseases and disorders of the skin: psoriasis | Blood, serum, CSF, urine, synovial fluid, tears |
| Urological disorders: benign prostatic hypertrophy (BPH), | Blood, serum, urine |
| polycystic kidney disease, interstitial cystitis | |
| Hepatic disease/injury: Cirrhosis, induced hepatotoxicity | Blood, serum, urine |
| (due to exposure to natural or synthetic chemical sources) | |
| Kidney disease/injury: acute, sub-acute, chronic | Blood, serum, urine |
| conditions, Podocyte injury, focal segmental | |
| glomerulosclerosis | |
| Endometriosis | Blood, serum, urine, vaginal fluids |
| Osteoporosis | Blood, serum, urine, synovial fluid |
| Pancreatitis | Blood, serum, urine, pancreatic juice |
| Asthma | Blood, serum, urine, sputum, bronchiolar lavage fluid |
| Allergies | Blood, serum, urine, sputum, bronchiolar lavage fluid |
| Prion-related diseases | Blood, serum, CSF, urine |
| Viral Infections: HIV/AIDS | Blood, serum, urine |
| Sepsis | Blood, serum, urine, tears, nasal lavage |
| Organ rejection/transplantation | Blood, serum, urine, various lavage fluids |
| Differentiating conditions: adenoma versus hyperplastic | Blood, serum, urine, sputum, feces, colonic lavage fluid |
| polyp, irritable bowel syndrome (IBS) versus normal, | |
| classifying Dukes stages A, B, C, and/or D of colon | |
| cancer, adenoma with low-grade hyperplasia versus high- | |
| grade hyperplasia, adenoma versus normal, colorectal | |
| cancer versus normal, IBS versus. ulcerative colitis (UC) | |
| versus Crohn's disease (CD), | |
| Pregnancy related physiological states, conditions, or | Maternal serum, amniotic fluid, cord blood |
| affiliated diseases: genetic risk, adverse pregnancy | |
| outcomes | |

The biological samples may be obtained through a third party, such as a party not performing the analysis of the exosome. For example, the sample may be obtained through a clinician, physician, or other health care manager of a subject from which the sample is derived. Alternatively, the biological sample may obtained by the same party analyzing the exosomes.

The volume of the biological sample used for analyzing an exosome can be in the range of between 0.1-20 mL, such as less than about 20, 15, 10, 9, 8, 7, 6, 5, 4, 3, 2, 1 or 0.1 mL.

Furthermore, analysis of one or more exosomes in a biological sample can be used to determine whether an additional biological sample should be obtained for analysis. For example, analysis of one or more exosomes in a serum sample can be used to determine whether a biopsy should be obtained.

### Exosomes

The exosomes from a biological sample for analysis are used to determine a phenotype. Exosomes are small vesicles that are released into the extracellular environment from a variety of different cells such as but not limited to, cells that originate from, or are derived from, the ectoderm, endoderm, or mesoderm including any such cells that have undergone genetic, environmental, and/or any other variations or alterations (e.g. Tumor cells or cells with genetic mutations). An exosome is typically created intracellularly when a segment of the cell membrane spontaneously invaginates and is ultimately exocytosed (see for example, Keller et al., Immunol. Lett. 107 (2): 102-8 (2006)). Exosomes can have, but not be limited to, a diameter of greater than about 10, 20, or 30 nm. They can have a diameter of about 30-1000 nm, about 30-800 nm, about 30-200 nm, or about 30-100 nm. In some embodiments, the exosomes can have, but not be limited to, a diameter of less than about 10,000 nm, 1000 nm, 800 nm, 500 nm, 200 nm, 100 nm or 50 nm. As used throughout, the term “about,” when referring to a value or to an amount is meant to encompass variations in some embodiments ±10% from the specified amount, as such variations are appropriate.

Exosomes may also be referred to as microvesicles, nanovesicles, vesicles, dexosomes, bleb, blebby, prostasomes, microparticles, intralumenal vesicles, endosomal-like vesicles or exocytosed vehicles. As used herein, exosomes can also include any shed membrane bound particle that is derived from either the plasma membrane or an internal membrane. Exosomes can also include cell-derived structures bounded by a lipid bilayer membrane arising from both herniated evagination (blebbing) separation and sealing of portions of the plasma membrane or from the export of any intracellular membrane-bounded vesicular structure containing various membrane-associated proteins of tumor origin, including surface-bound molecules derived from the host circulation that bind selectively to the tumor-derived proteins together with molecules contained in the exosome lumen, including but not limited to tumor-derived microRNAs or intracellular proteins. Blebs and blebbing are further described in Charras et al., Nature Reviews Molecular and Cell Biology, Vol. 9, No. 11, p. 730-736 (2008). Exosomes can also include membrane fragments. Circulating tumor-derived exosomes (CTEs) as referenced herein are exosomes that are shed into circulation or bodily fluids from tumor cells. CTEs, as with cell-of-origin specific exosomes, typically have unique biomarkers that permit their isolation from bodily fluids in a highly specific manner.

Exosomes can be directly assayed from the biological samples, such that the level of exosomes is determined or the one or more biomarkers of the exosomes is determined without prior isolation, purification, or concentration of the exosomes. Alternatively, exosomes may be isolated, purified, or concentrated from a sample prior to analysis.

### Isolation of Exosomes

An exosome may be purified or concentrated prior to analysis. Analysis of an exosome can include quantitiating the amount one or more exosome populations of a biological sample. For example, a heterogeneous population of exosomes can be quantitated, or a homogeneous population of exosomes, such as a population of exosomes with a particular biomarker profile, a particular bio-signature, or derived from a particular cell type (cell-of-origin specific exosomes) can be isolated from a heterogeneous population of exosomes and quantitated. Analysis of an exosome can also include detecting, quantitatively or qualitatively, a particular biomarker profile or a bio-signature, of an exosome, as described below.

An exosome can be stored and archived, such as in a bio-fluid bank and retrieved for analysis as necessary. An exosome may also be isolated from a biological sample that has been previously harvested and stored from a living or deceased subject. In addition, an exosome may be isolated from a biological sample which has been collected as described in King et al., Breast Cancer Res 7(5): 198-204 (2005). An exosome may be isolated from an archived or stored sample. Alternatively, an exosome may be isolated from a biological sample and analyzed without storing or archiving of the sample. Furthermore, a third party may obtain or store the biological sample, or obtain or store the exosomes for analysis.

An enriched population of exosomes can be obtained from a biological sample. For example, exosomes may be concentrated or isolated from a biological sample using size exclusion chromatography, density gradient centrifugation, differential centrifugation, nanomembrane ultrafiltration, immunoabsorbent capture, affinity purification, microfluidic separation, or combinations thereof.

Size exclusion chromatography, such as gel permeation columns, centrifugation or density gradient centrifugation, and filtration methods can be used. For example, exosomes can be isolated by differential centrifugation, anion exchange and/or gel permeation chromatography (for example, as described in U.S. Pat. Nos. 6,899,863 and 6,812,023), sucrose density gradients, organelle electrophoresis (for example, as described in U.S. Pat. No. 7,198,923), magnetic activated cell sorting (MACS), or with a nanomembrane ultrafiltration concentrator. Various combinations of isolation or concentration methods can be used.

Highly abundant proteins, such as albumin and immunoglobulin, may hinder isolation of exosomes from a biological sample. For example, exosomes may be isolated from a biological sample using a system that utilizes multiple antibodies that are specific to the most abundant proteins found in blood. Such a system can remove up to several proteins at once, thus unveiling the lower abundance species such as cell-of-origin specific exosomes.

This type of system can be used for isolation of exosomes from biological samples such as blood, cerebrospinal fluid or urine. The isolation of exosomes from a biological sample may also be enhanced by high abundant protein removal methods as described in Chromy et al. J Proteome Res 2004; 3:1120-1127. In another embodiment, the isolation of exosomes from a biological sample may also be enhanced by removing serum proteins using glycopeptide capture as described in Zhang et al, Mol Cell Proteomics 2005; 4:144-155. In addition, exosomes from a biological sample such as urine may be isolated by differential centrifugation followed by contact with antibodies directed to cytoplasmic or anti-cytoplasmic epitopes as described in Pisitkun et al., Proc Natl Acad Sci USA, 2004; 101:13368-13373.

Isolation or enrichment of exosomes from biological samples can also be enhanced by use of sonication (for example, by applying ultrasound), or the use of detergents, other membrane-active agents, or any combination thereof. For example, ultrasonic energy can be applied to a potential tumor site, and without being bound by theory, release of exosomes from the tissue can be increased, allowing an enriched population of exosomes that can be analyzed or assessed from a biological sample using one or more methods disclosed herein.

Binding Agents

A binding agent is an agent that binds to an exosomal component, such as a biomarker of an exosome. The binding agent can be a capture agent. A capture agent captures the exosome by binding to an exosomal target, such as a biomarker on the exosome. For example, the capture agent can be a capture antibody that binds to an antigen on the exosome. The capture agent can be coupled to a substrate and used to isolate the exosome, such as described herein.

A binding agent can be used after exosomes are concentrated or isolated from a biological sample. For example, exosomes can first be isolated from a biological sample before exosomes with a specific biomarker are isolated using a binding agent for the biomarker. Thus, exosomes with the specific biomarker is isolated from a heterogeneous population of exosomes. Alternatively, a binding agent may be used on a biological sample comprising exosomes without a prior isolation step or concentration of exosomes. For example, a binding agent is used to isolate an exosome with a specific biomarker from a biological sample.

A binding agent can be DNA, RNA, monoclonal antibodies, polyclonal antibodies, Fabs, Fab′, single chain antibodies, synthetic antibodies, aptamers (DNA/RNA), peptoids, zDNA, peptide nucleic acids (PNAs), locked nucleic acids (LNAs), lectins, synthetic or naturally occurring chemical compounds (including but not limited to drugs, labeling reagents), dendrimers, or combinations thereof. For example, the binding agent can be a capture antibody.

In some instances, a single binding agent can be employed to isolate an exosome. In other instances, a combination of different binding agents may be employed to isolate an exosome. For example, at least 2, 3, 4, 5, 6, 7, 8, 9, 10, 11, 12, 13, 14, 15, 16, 17, 18, 19, 20, 25, 50, 75 or 100 different binding agents may be used to isolate an exosome from a biological sample. Furthermore, the one or more different binding agents for an exosome can form the bio-signature of the exosome, further described below.

Different binding agents can also be used for multiplexing. For example, isolation of more than one population of exosomes (for example, exosomes from specific cell types) can be performed by isolating each exosome population with a different binding agent. Different binding agents can be bound to different particles, wherein the different particles are labeled. In another embodiment, an array comprising different binding agents can be used for multiplex analysis, wherein the different binding agents are differentially labeled or can be ascertained based on the location of the binding agent on the array. Multiplexing can be accomplished up to the resolution capability of the labels or detection method, such as described below.

The binding agent can be an antibody. For example, an exosome may be isolated using one or more antibodies specific for one or more antigens present on the exosome. For example, an exosome can have CD63 on its surface, and an antibody, or capture antibody, for CD63 can be used to isolate the exosome. Alternatively, an exosome derived from a tumor cell can express EpCam, the exosome can be isolated using an antibody for EpCam and CD63. Other antibodies for isolating exosomes can include an antibody, or capture antibody, to CD9, PSCA, TNFR, CD63, B7H3, MFG-E8, EpCam, Rab, CD81, STEAP, PCSA, PSMA, or 5T4.

The antibodies disclosed herein can be immunoglobulin molecules or immunologically active portions of immunoglobulin molecules, i.e., molecules that contain an antigen binding site that specifically binds an antigen and synthetic antibodies. The immunoglobulin molecules can be of any class (e.g., IgG, IgE, IgM, IgD or IgA) or subclass of immunoglobulin molecule. Antibodies include, but are not limited to, polyclonal, monoclonal, bispecific, synthetic, humanized and chimeric antibodies, single chain antibodies, Fab fragments and F(ab′)₂ fragments, Fv or Fv′ portions, fragments produced by a Fab expression library, anti-idiotypic (anti-Id) antibodies, or epitope-binding fragments of any of the above. An antibody, or generally any molecule, “binds specifically” to an antigen (or other molecule) if the antibody binds preferentially to the antigen, and, e.g., has less than about 30%, 20%, 10%, 5% or 1% cross-reactivity with another molecule.

The binding agent can also be a polypeptide or peptide. Polypeptide is used in its broadest sense and may include a sequence of subunit amino acids, amino acid analogs, or peptidomimetics. The subunits may be linked by peptide bonds. The polypeptides may be naturally occurring, processed forms of naturally occurring polypeptides (such as by enzymatic digestion), chemically synthesized or recombinantly expressed. The polypeptides for use in the methods of the present invention may be chemically synthesized using standard techniques. The polypeptides may comprise D-amino acids (which are resistant to L-amino acid-specific proteases), a combination of D- and L-amino acids, β amino acids, or various other designer or non-naturally occurring amino acids (e.g., β-methyl amino acids, Cα-methyl amino acids, and Nα-methyl amino acids, etc.) to convey special properties. Synthetic amino acids may include ornithine for lysine, and norleucine for leucine or isoleucine. In addition, the polypeptides can have peptidomimetic bonds, such as ester bonds, to prepare polypeptides with novel properties. For example, a polypeptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a polypeptide would be resistant to protease activity, and would possess an extended half-live in vivo. Polypeptides can also include peptoids (N-substituted glycines), in which the side chains are appended to nitrogen atoms along the molecule's backbone, rather than to the a-carbons, as in amino acids. Polypeptides and peptides are intended to be used interchangeably throughout this application, i.e. where the term peptide is used, it may also include polypeptides and where the term polypeptides is used, it may also include peptides.

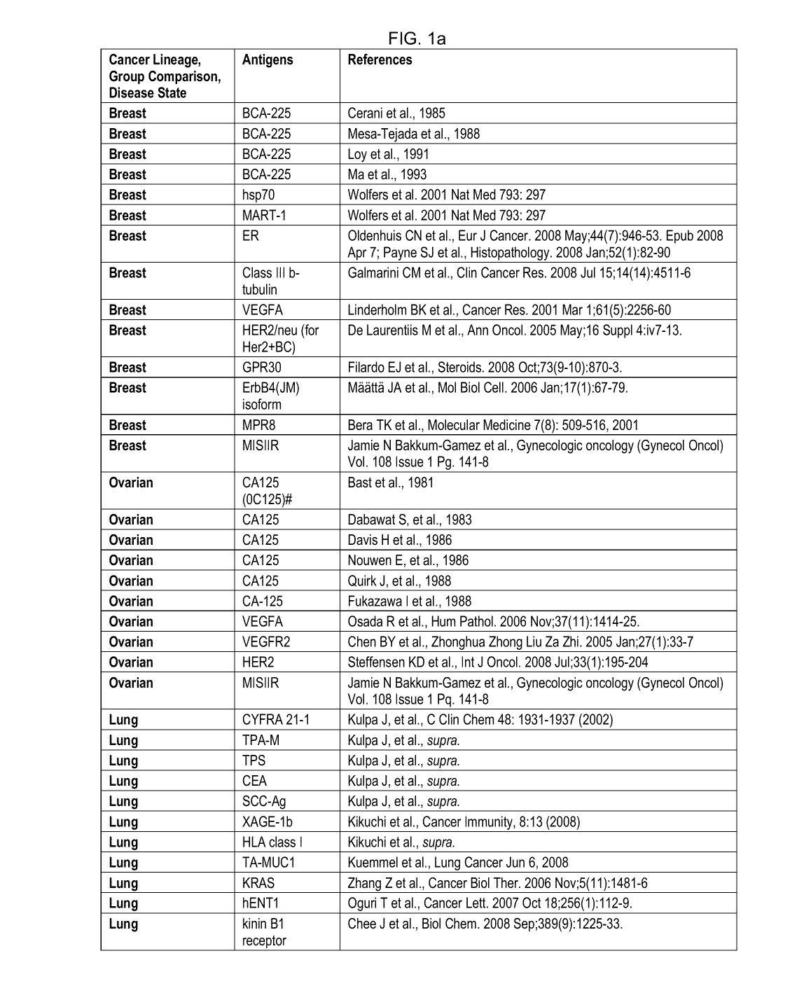

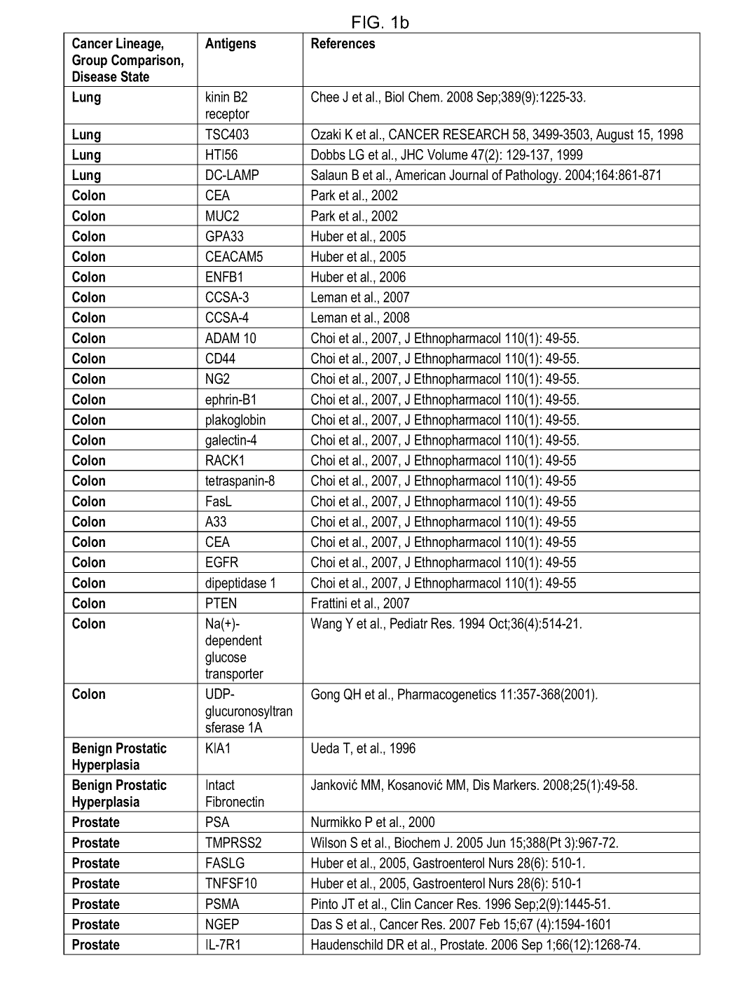

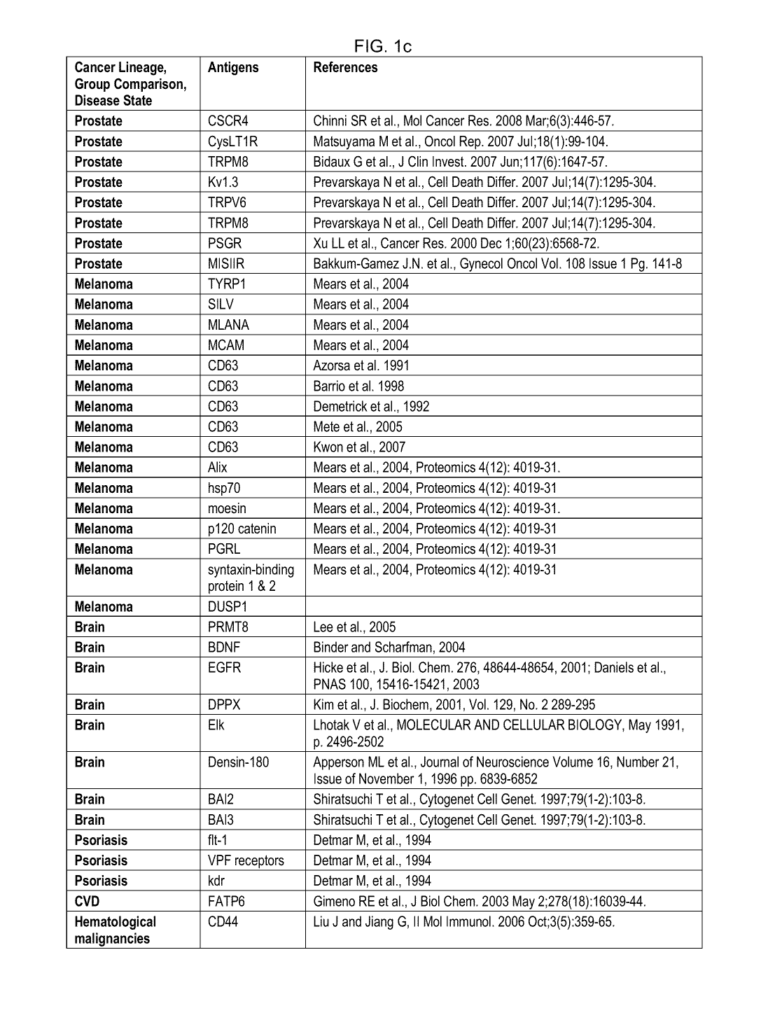

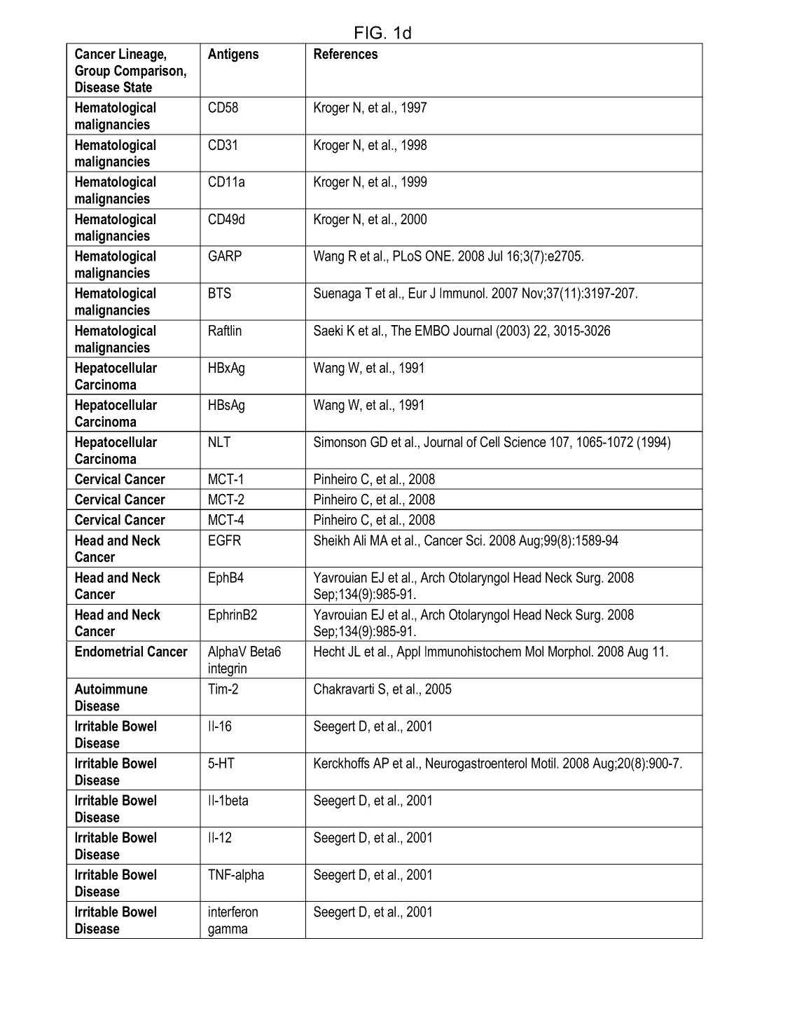

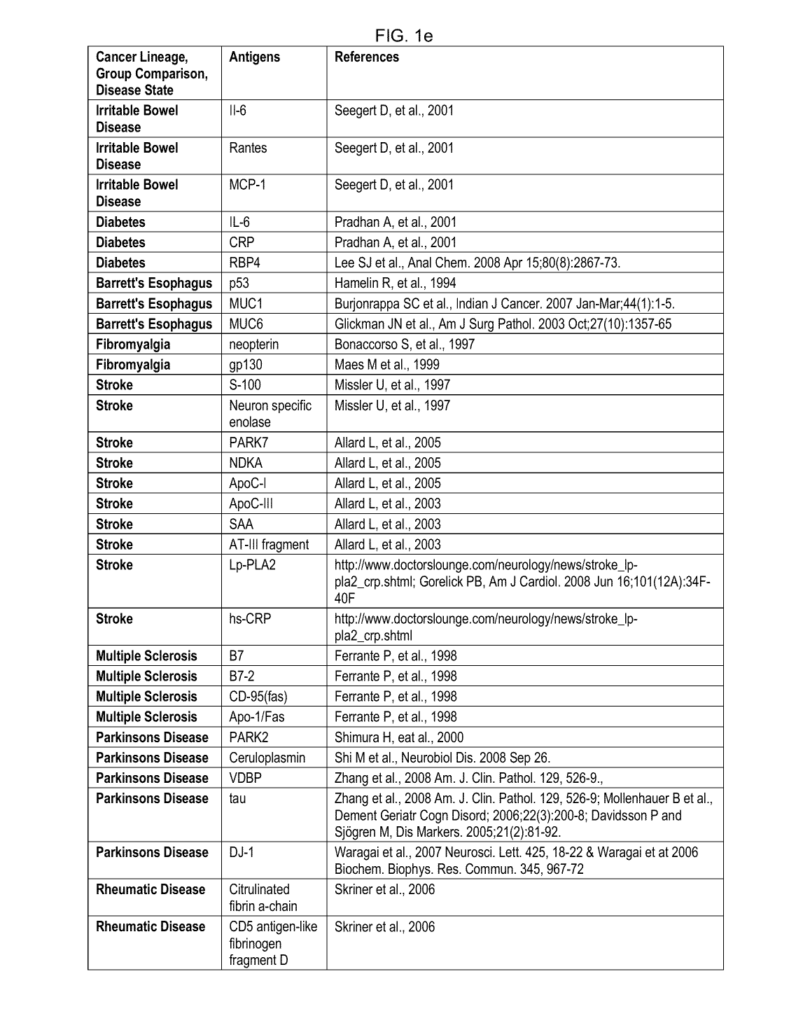

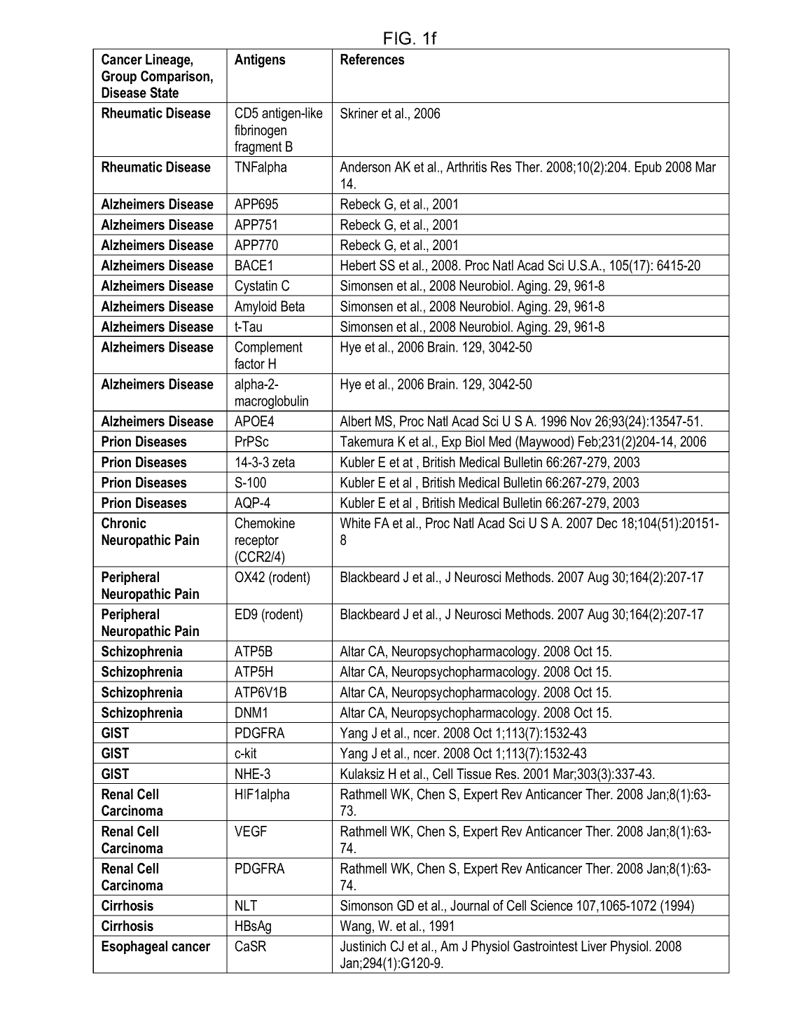

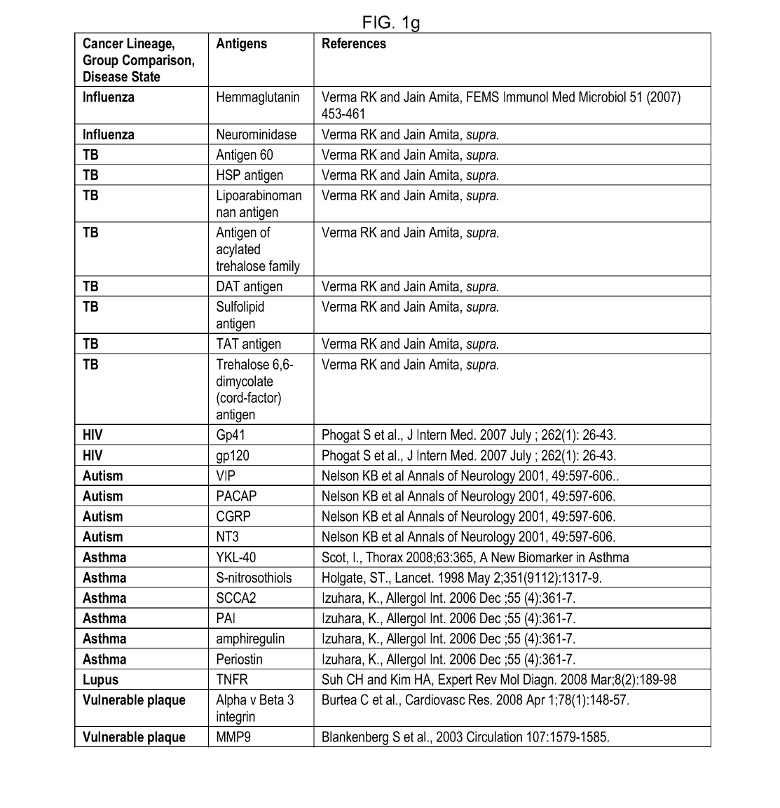

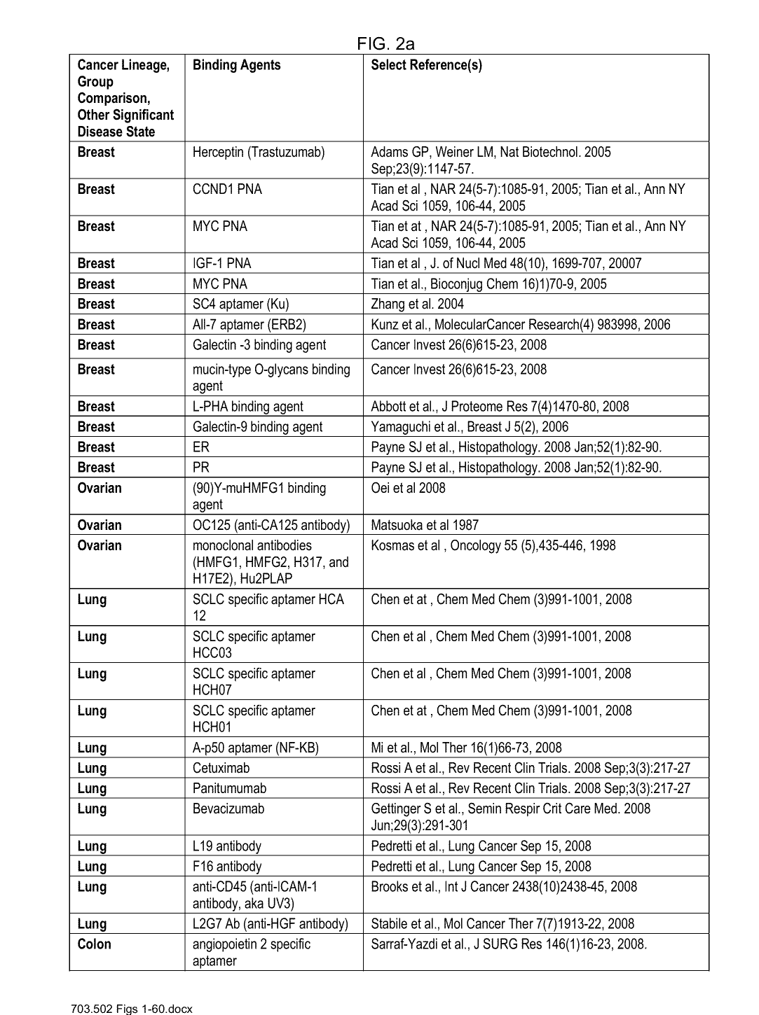

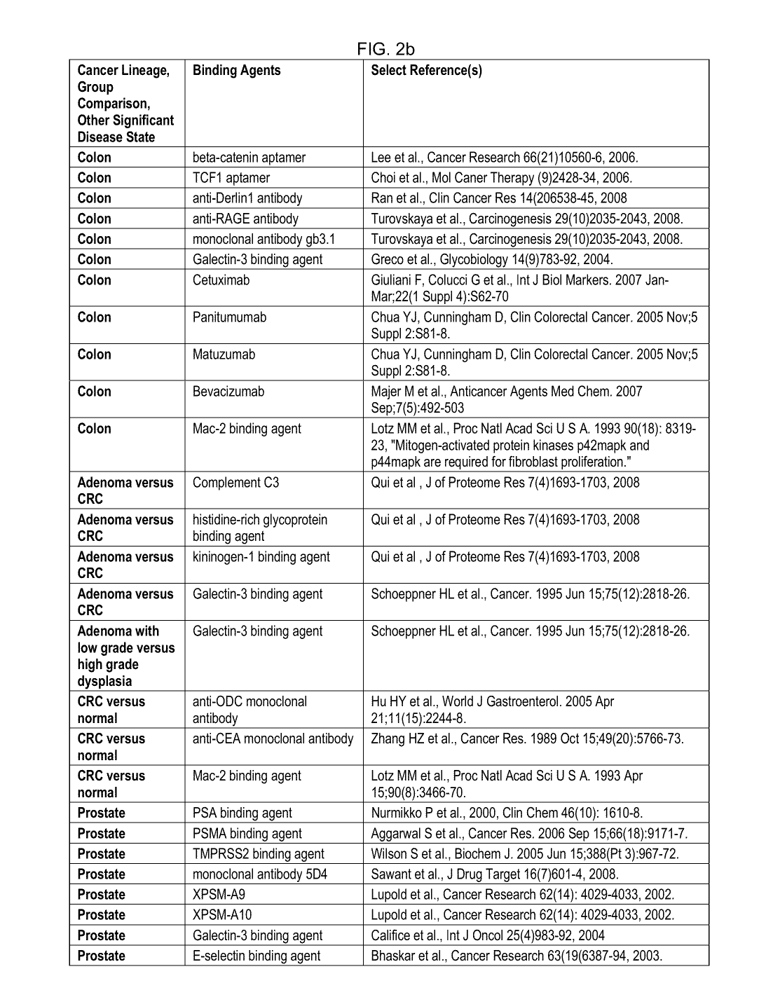

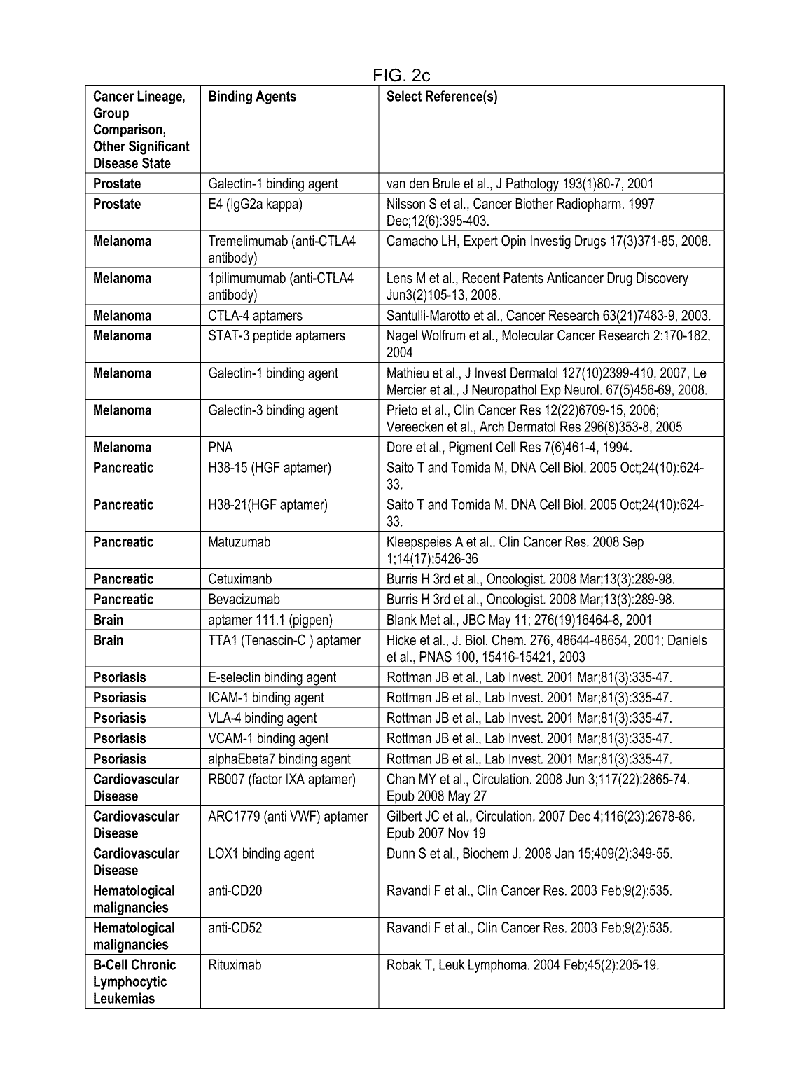

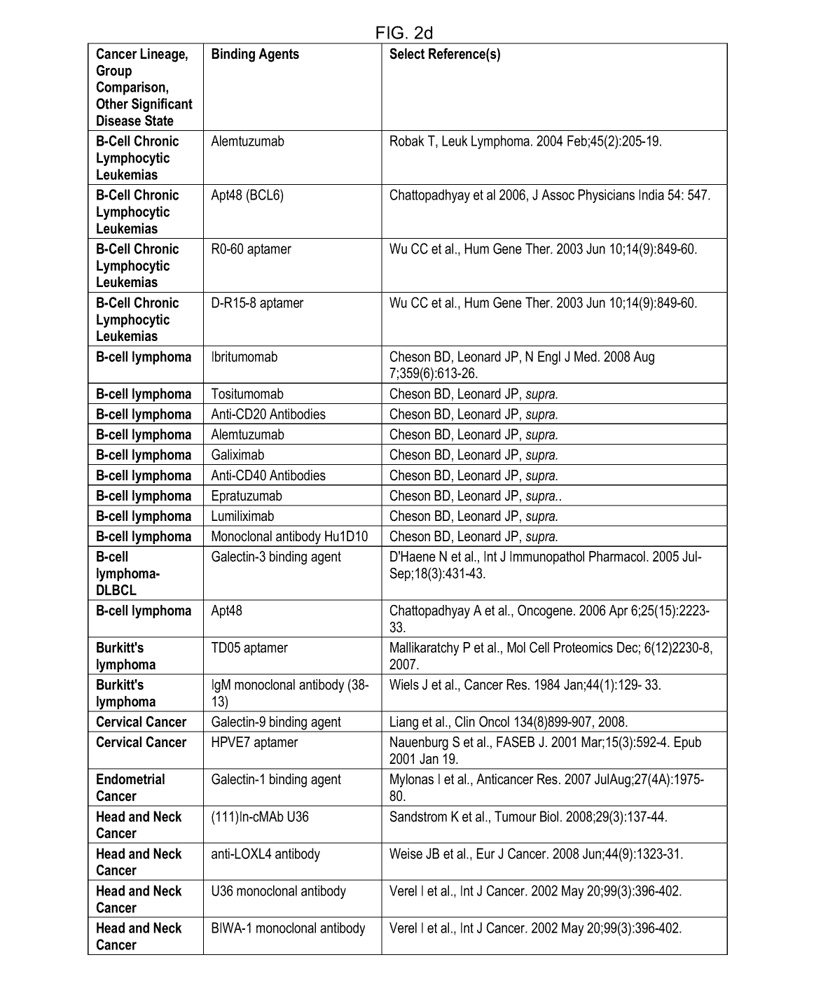

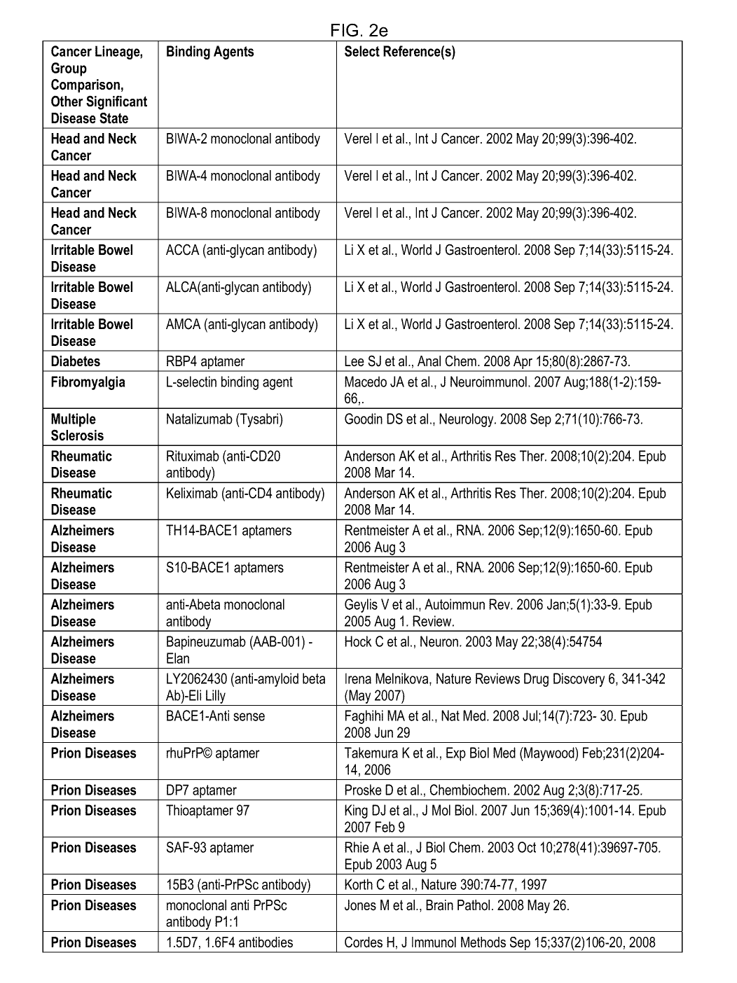

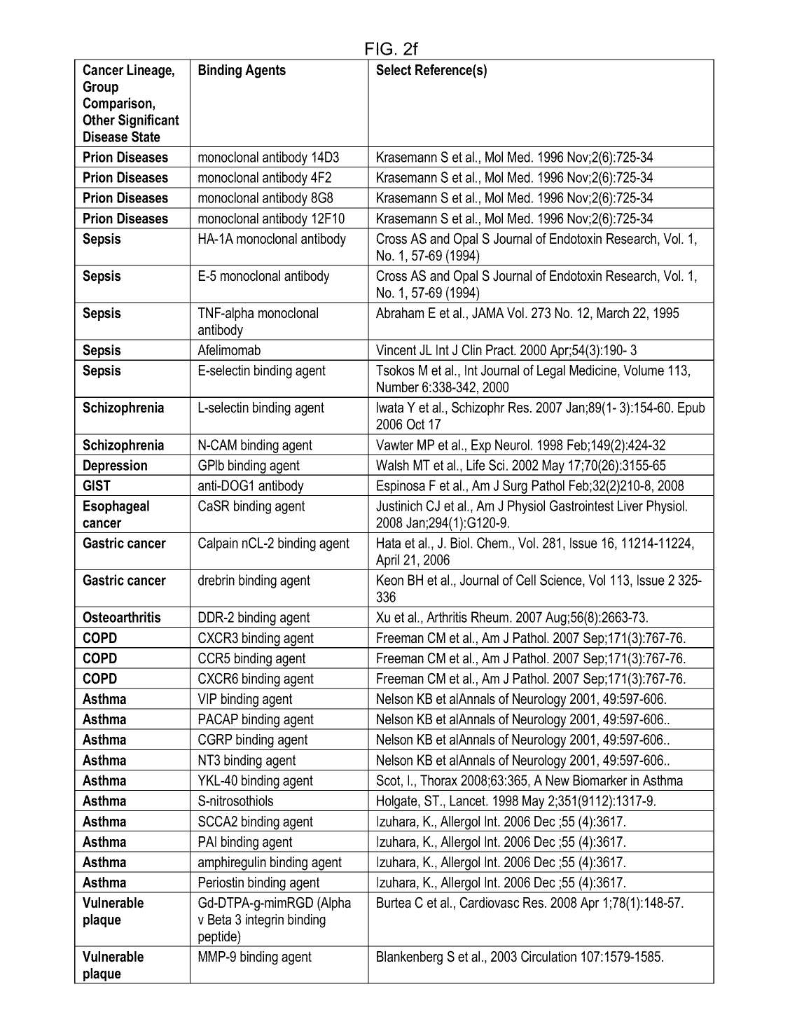

An exosome may be isolated using a known binding agent. For example, the binding agent can be an agent that binds exosomal “housekeeping proteins,” or general exosome biomarkers, such as CD63, CD9, CD81, CD82, CD37, CD53, or Rab-5b. The binding agent can also be an agent that binds to exosomes derived from specific cell types, such as tumor cells (e.g. binding agent for EpCam) or specific cell-of-origins, such as described below. For example, the binding agent used to isolate an exosome may be a binding agent for an antigen selected from FIG. 1. The binding agent for an exosome can also be selected from those listed in FIG. 2. For example, the binding agent can be for an antigen such as 5T4, B7H3, caveolin, CD63, CD9, E-Cadherin, MFG-E8, PSCA, PSMA, Rab-5B, STEAP, TNFR1, CD81, EpCam, CD59, or CD66. One or more binding agents, such as one or more binding agents for two or more of the antigens, can be used for isolating an exosome. The binding agent used can be selected based on the desire of isolating exosomes derived from particular cell types, or cell-of-origin specific exosomes.

A binding agent can also be linked directly or indirectly to a solid surface or substrate. A solid surface or substrate can be any physically separable solid to which a binding agent can be directly or indirectly attached including, but not limited to, surfaces provided by microarrays and wells, particles such as beads, columns, optical fibers, wipes, glass and modified or functionalized glass, quartz, mica, diazotized membranes (paper or nylon), polyformaldehyde, cellulose, cellulose acetate, paper, ceramics, metals, metalloids, semiconductive materials, quantum dots, coated beads or particles, other chromatographic materials, magnetic particles; plastics (including acrylics, polystyrene, copolymers of styrene or other materials, polypropylene, polyethylene, polybutylene, polyurethanes, TEFLON™, etc.), polysaccharides, nylon or nitrocellulose, resins, silica or silica-based materials including silicon and modified silicon, carbon, metals, inorganic glasses, plastics, ceramics, conducting polymers (including polymers such as polypyrole and polyindole); micro or nanostructured surfaces such as nucleic acid tiling arrays, nanotube, nanowire, or nanoparticulate decorated surfaces; or porous surfaces or gels such as methacrylates, acrylamides, sugar polymers, cellulose, silicates, or other fibrous or stranded polymers. In addition, as is known the art, the substrate may be coated using passive or chemically-derivatized coatings with any number of materials, including polymers, such as dextrans, acrylamides, gelatins or agarose. Such coatings can facilitate the use of the array with a biological sample.

For example, an antibody used to isolate an exosome can be bound to a solid substrate such as a well, such as commercially available plates (e.g. from Nunc, Milan Italy). Each well can be coated with the antibody. In some embodiments, the antibody used to isolate an exosome can be bound to a solid substrate such as an array. The array can have a predetermined spatial arrangement of molecule interactions, binding islands, biomolecules, zones, domains or spatial arrangements of binding islands or binding agents deposited within discrete boundaries. Further, the term array may be used herein to refer to multiple arrays arranged on a surface, such as would be the case where a surface bore multiple copies of an array. Such surfaces bearing multiple arrays may also be referred to as multiple arrays or repeating arrays.

A binding agent can also be bound to particles such as beads or microspheres. For example, an antibody specific for an exosomal component can be bound to a particle, and the antibody-bound particle is used to isolate exosomes from a biological sample. In some embodiments, the microspheres may be magnetic or fluorescently labeled. In addition, a binding agent for isolating exosomes can be a solid substrate itself. For example, latex beads, such as aldehyde/sulfate beads (Interfacial Dynamics, Portland, Oreg.) can be used.

A binding agent bound to a magnetic bead can also be used to isolate an exosome. For example, a biological sample such as serum from a patient can be collected for colon cancer screening. The sample can be incubated with anti-CCSA-3 (Colon Cancer-Specific Antigen) coupled to magnetic microbeads. A low-density microcolumn can be placed in the magnetic field of a MACS Separator and the column is then washed with a buffer solution such as Tris-buffered saline. The magnetic immune complexes can then be applied to the column and unbound, non-specific material can be discarded. The CCSA-3 selected exosomes can be recovered by removing the column from the separator and placing it on a collection tube. A buffer can be added to the column and the magnetically labeled exosomes can be released by applying the plunger supplied with the column. The isolated exosomes can be diluted in IgG elution buffer and the complex can then be centrifuged to separate the microbeads from the exosomes. The pelleted isolated cell-of-origin specific exosomes can be resuspended in buffer such as phosphate-buffered saline and quantitated. Alternatively, due to the strong adhesion force between the antibody captured cell-of-origin specific exosomes and the magnetic microbeads, a proteolytic enzyme such as trypsin can be used for the release of captured exosomes without the need for centrifugation. The proteolytic enzyme can be incubated with the antibody captured cell-of-origin specific exosomes for at least a time sufficient to release the exosomes.

A binding agent, such as an antibody, for isolating an exosome is preferably contacted with the biological sample comprising the exosome of interest for at least a time sufficient for the binding agent to bind to an exosomal component. For example, an antibody may be contacted with a biological sample for various intervals ranging from seconds days, including but not limited to, about 10 minutes, 30 minutes, 1 hour, 3 hours, 5 hours, 7 hours, 10 hours, 15 hours, 1 day, 3 days, 7 days or 10 days.

A binding agent, such as an antibody specific to an antigen listed in FIG. 1, or a binding agent listed in FIG. 2, can be labeled with, including but not limited to, a magnetic label, a fluorescent moiety, an enzyme, a chemiluminescent probe, a metal particle, a non-metal colloidal particle, a polymeric dye particle, a pigment molecule, a pigment particle, an electrochemically active species, semiconductor nanocrystal or other nanoparticles including quantum dots or gold particles. The label can be, but not be limited to, fluorophores, quantum dots, or radioactive labels. For example, the label can be a radioisotope (radionuclides), such as ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi. The label can be a fluorescent label, such as a rare earth chelate (europium chelate), fluorescein type, such as, but not limited to, FITC, 5-carboxyfluorescein, 6-carboxy fluorescein; a rhodamine type, such as, but not limited to, TAMRA; dansyl; Lissamine; cyanines; phycoerythrins; Texas Red; and analogs thereof.

A binding agent can be directly or indirectly labeled, e.g., the label is attached to the antibody through biotin-streptavidin. Alternatively, an antibody is not labeled, but is later contacted with a second antibody that is labeled after the first antibody is bound to an antigen of interest.

For example, various enzyme-substrate labels are available or disclosed (see for example, U.S. Pat. No. 4,275,149). The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP), β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of enzyme-substrate combinations include, but are not limited to, horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB)); alkaline phosphatase (AP) with para-nitrophenyl phosphate as chromogenic substrate; and β-D-galactosidase (β-D-Gal) with a chromogenic substrate (e.g., p-nitrophenyl-β-D-galactosidase) or fluorogenic substrate 4-methylumbelliferyl-β-D-galactosidase.

Depending on the method of isolation used, the binding agent may be linked to a solid surface or substrate, such as arrays, particles, wells and other substrates described above. Methods for direct chemical coupling of antibodies, to the cell surface are known in the art, and may include, for example, coupling using glutaraldehyde or maleimide activated antibodies. Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxysuccinimide. Succinimide groups react effectively with amino groups at pH values above 7, and preferentially between about pH 8.0 and about pH 8.5. Biotinylation can be accomplished by, for example, treating the cells with dithiothreitol followed by the addition of biotin maleimide.

Flow Cytometry

Isolation of exosomes using particles such as beads or microspheres can also be performed using flow cytometry. Flow cytometry can be used for sorting microscopic particles suspended in a stream of fluid. As particles pass through they can be selectively charged and on their exit can be deflected into separate paths of flow. It is therefore possible to separate populations from an original mix, such as a biological sample, with a high degree of accuracy and speed. Flow cytometry allows simultaneous multiparametric analysis of the physical and/or chemical characteristics of single cells flowing through an optical/electronic detection apparatus. A beam of light, usually laser light, of a single frequency (color) is directed onto a hydrodynamically focused stream of fluid. A number of detectors are aimed at the point where the stream passes through the light beam; one in line with the light beam (Forward Scatter or FSC) and several perpendicular to it (Side Scatter or SSC) and one or more fluorescent detectors.

Each suspended particle passing through the beam scatters the light in some way, and fluorescent chemicals in the particle may be excited into emitting light at a lower frequency than the light source. This combination of scattered and fluorescent light is picked up by the detectors, and by analyzing fluctuations in brightness at each detector (one for each fluorescent emission peak), it is possible to deduce various facts about the physical and chemical structure of each individual particle. FSC correlates with the cell size and SSC depends on the inner complexity of the particle, such as shape of the nucleus, the amount and type of cytoplasmic granules or the membrane roughness. Some flow cytometers have eliminated the need for fluorescence and use only light scatter for measurement.

Flow cytometers can analyze several thousand particles every second in “real time” and can actively separate out and isolate particles having specified properties. They offer high-throughput automated quantification, and separation, of the set parameters for a high number of single cells during each analysis session. Modern instruments have multiple lasers and fluorescence detectors, for example up to 4 lasers and 18 fluorescence detectors, allowing multiple labels to be used to more precisely specify a target population by their phenotype.

The data resulting from flow-cytometers can be plotted in 1 dimension to produce histograms or seen in 2 dimensions as dot plots or in 3 dimensions with newer software. The regions on these plots can be sequentially separated by a series of subset extractions which are termed gates. Specific gating protocols exist for diagnostic and clinical purposes especially in relation to hematology. The plots are often made on logarithmic scales. Because different fluorescent dye's emission spectra overlap, signals at the detectors have to be compensated electronically as well as computationally. Fluorophores for labeling biomarkers may include those described in Ormerod, Flow Cytometry 2nd ed., Springer-Verlag, New York (1999), and in Nida et al., Gynecologic Oncology 2005; 4 889-894 which is incorporated herein by reference.

Multiplexing

Different binding agents can be used for multiplexing different exosome populations. Different exosome populations can be isolated or detected using different binding agents. Each exosome population in a biological sample can be labeled with a different signaling label, such as fluorophores, quantum dots, or radioactive labels, such as described above. The label can be directly conjugated to a binding agent or indirectly used to detect a binding agent. The number of populations detected in a multiplexing assay is dependent on the resolution capability of the labels and the summation of signals, as more than two differentially labeled exosome populations that bind two or more affinity elements can produce summed signals.

Multiplexing of at least 2, 3, 4, 5, 6, 7, 8, 9, 10, 11, 12, 13, 14, 15, 16, 17, 18, 19, 20, 25, 50, 75 or 100 different exosome populations may be performed. For example, one population of exosomes specific to a cell-of-origin can be assayed along with a second population of exosomes specific to a different cell-of-origin, where each population is labeled with a different label. Alternatively, a population of exosomes with a particular biomarker or bio-signature can be assayed along with a second population of exosomes with a different biomarker or bio-signature.

In one embodiment, multiplex analysis is performed by applying a plurality of exosomes comprising more than one population of exosomes to a plurality of substrates, such as beads. Each bead is coupled to one or more capture agents. The plurality of beads is divided into subsets, where beads with the same capture agent or combination of capture agents form a subset of beads, such that each subset of beads has a different capture agent or combination of capture agents than another subset of beads. The beads can then be used to capture exosomes that comprises a component that binds to the capture agent. The different subsets can be used to capture different populations of exosomes. The captured exosomes can then be analyzed by detecting one or more biomarkers of the exosomes.

Flow cytometry can be used in combination with a particle-based or bead based assay. Multiparametric immunoassays or other high throughput detection assays using bead coatings with cognate ligands and reporter molecules with specific activities consistent with high sensitivity automation can be used. For example, beads in each subset can be differentially labeled from another subset. For example, in a particle based assay system, a binding agent or capture agent for an exosome, such as a capture antibody, can be immobilized on addressable beads or microspheres. Each binding agent for each individual binding assay (such as an immunoassay when the binding agent is an antibody) can be coupled to a distinct type of microsphere (i.e., microbead) and the binding assay reaction takes place on the surface of the microspheres. Microspheres can be distinguished by different labels, for example, a microsphere with a specific capture agent would have a different signaling label as compared to another microsphere with a different capture agent. For example, microspheres can be dyed with discrete fluorescence intensities such that the fluorescence intensity of a microsphere with a specific binding agent is different than that of another microsphere with a different binding agent.

The microsphere can be labeled or dyed with at least 2 different labels or dyes. In some embodiments, the microsphere is labeled with at least 3, 4, 5, 6, 7, 8, 9, or 10 different labels. Different microspheres in a plurality of microspheres can have more than one label or dye, wherein various subsets of the microspheres have various ratios and combinations of the labels or dyes permitting detection of different microspheres with different binding agents. For example, the various ratios and combinations of labels and dyes can permit different fluorescent intensities. Alternatively, the various ratios and combinations maybe used to generate different detection patters to identify the binding agent. The microspheres can be labeled or dyed externally or may have intrinsic fluorescence or signaling labels. Beads can be loaded separately with their appropriate binding agents and thus, different exosome populations can be isolated based on the different binding agents on the differentially labeled microspheres to which the different binding agents are coupled.

In another embodiment, multiplex analysis can be performed using a planar substrate, wherein the said substrate comprises a plurality of capture agents. The plurality of capture agents can capture one or more populations of exosomes, and one or more biomarkers of the captured exosomes detected. The planar substrate can be a microarray or other substrate as further described herein.

Novel Binding Agents

An exosome may be isolated using a binding agent for a novel component of an exosome, such as an antibody for a novel antigen specific to an exosome of interest. Novel antigens that are specific to exosomes of interest may be isolated or identified using different test compounds of known composition bound to a substrate, such as an array or a plurality of particles, which can allow a large amount of chemical/structural space to be adequately sampled using only a small fraction of the space. The novel antigen identified can also serve as a biomarker for the exosome. For example, a novel antigen identified for a cell-of-origin specific exosome can be a biomarker for that particular cell-of-origin specific exosome.

A binding agent can be identified by screening either a homogeneous or heterogeneous exosome population against test compounds. Since the composition of each test compound on the substrate surface is known, this constitutes a screen for affinity elements. For example, a test compound array comprises test compounds at specific locations on the substrate addressable locations, and can be used to identify one or more binding agents for an exosome. The test compounds can all be unrelated or related based on minor variations of a core sequence or structure. The different test compounds may include variants of a given test compound (such as polypeptide isoforms), test compounds that are structurally or compositionally unrelated, or a combination thereof.

Test compounds can be peptoids, polysaccharides, organic compounds, inorganic compounds, polymers, lipids, nucleic acids, polypeptides, antibodies, proteins, polysaccharides, or other compounds. The test compounds can be natural or synthetic. The test compounds can comprise or consist of linear or branched heteropolymeric compounds based on any of a number of linkages or combinations of linkages (e.g., amide, ester, ether, thiol, radical additions, metal coordination, etc.), dendritic structures, circular structures, cavity structures or other structures with multiple nearby sites of attachment that serve as scaffolds upon which specific additions are made. These test compounds can be spotted on the substrate or synthesized in situ, using standard methods in the art. In addition, the test compounds can be spotted or synthesized in situ in combinations in order to detect useful interactions, such as cooperative binding.

The test compounds can be polypeptides with known amino acid sequences, thus, detection a test compound binding with an exosome can lead to identification of a polypeptide of known amino sequence that can be used as a binding agent. For example, a homogenous population of exosomes can be applied to a spotted array on a slide containing between a few and 1,000,000 test polypeptides having a length of variable amino acids. The polypeptides can be attached to the surface through the C-terminus. The sequence of the polypeptides can be generated randomly from 19 amino acids, excluding cysteine. The binding reaction can include a non-specific competitor, such as excess bacterial proteins labeled with another dye such that the specificity ratio for each polypeptide binding target can be determined. The polypeptides with the highest specificity and binding can be selected. The identity of the polypeptide on each spot is known, and thus can be readily identified. Once the novel antigens specific to the homogeneous exosome population, such as a cell-of-origin specific exosome is identified, such cell-of-origin specific exosomes may subsequently be isolated using such antigens in methods described hereafter.

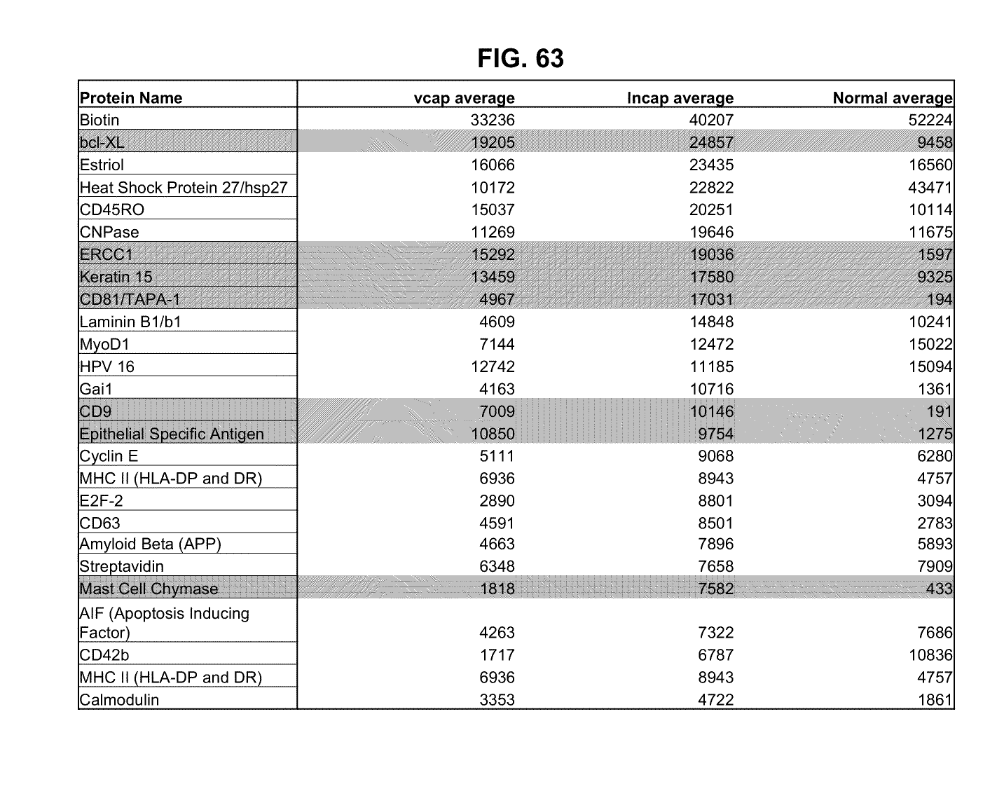

Arrays can also be used for identifying antibodies for isolating exosomes. Test antibodies can be attached to an array and screened against a heterogeneous population of exosomes to identify antibodies that can be used to isolate, and identify, an exosome. A homogeneous population of exosomes, such as cell-of-origin specific exosomes, can also be screened with an antibody array. Other than identifying antibodies to isolate the homogeneous population of exosomes, one or more protein biomarkers specific to the homogenous exosome population can be identified. Commercially available platforms with test antibodies pre-selected, or custom selection of test antibodies attached to the array, can be used. For example, an antibody array from Full Moon Biosystems can be screened using prostate cancer cell derived exosomes, identifying antibodies to Bcl-XL, ERCC1, Keratin 15, CD81/TAPA-1, CD9, Epithelial Specific Antigen (ESA), and Mast Cell Chymase as binding agents (see for example, FIG. 63), and the proteins identified can be used as biomarkers for the exosomes.

An antibody or synthetic antibody to be used as a binding agent can also be identified through a peptide array. Another method is the use of synthetic antibody generation through antibody phage display. M13 bacteriophage libraries of antibodies (e.g. Fabs) are displayed on the surfaces of phage particles as fusions to a coat protein. Each phage particle displays a unique antibody and also encapsulates a vector that contains the encoding DNA. Highly diverse libraries can be constructed and represented as phage pools, which can be used in antibody selection for binding to immobilized antigens. Antigen-binding phages are retained by the immobilized antigen, and the nonbinding phages are removed by washing. The retained phage pool can be amplified by infection of an Escherichia coli host and the amplified pool can be used for additional rounds of selection to eventually obtain a population that is dominated by antigen-binding clones. At this stage, individual phase clones can be isolated and subjected to DNA sequencing to decode the sequences of the displayed antibodies. Through the use of phase display and other methods known in the art, high affinity designer antibodies for exosomes can be generated.

Bead-based assays can also be used to identify novel binding agents to isolate exosomes. A test antibody or peptide can be conjugated to a particle. For example, a bead can be conjugated to an antibody or peptide and used to detect and quantify the proteins expressed on the surface of a population of exosomes in order to discover and specifically select for novel antibodies that can target exosomes from specific tissue or tumor types. Any molecule of organic origin can be successfully conjugated to a polystyrene bead through use of a commercially available kit according to manufacturer's instructions. Each bead set can be colored a certain detectable wavelength and each can be linked to a known antibody or peptide which can be used to specifically measure which beads are linked to exosomal proteins matching the epitope of previously conjugated antibodies or peptides. The beads can be dyed with discrete fluorescence intensities such that each bead with a different intensity has a different binding agent as described above.

For example, a purified exosome preparation can be diluted in assay buffer to an appropriate concentration according to empirically determined dynamic range of assay. A sufficient volume of coupled beads can be prepared and approximately 1 μl of the antibody-coupled beads can be aliqouted into a well and adjusted to a final volume of approximately 50 μl. Once the antibody-conjugated beads have been added to a vacuum compatible plate, the beads can be washed to ensure proper binding conditions. An appropriate volume of exosomal preparation can then be added to each well being tested and the mixture incubated, such as for 15-18 hours. A sufficient volume of detection antibodies using detection antibody diluent solution can be prepared and incubated with the mixture for 1 hour or for as long as necessary. The beads can then be washed before the addition of detection antibody (biotin expressing) mixture composed of streptavidin phycoereythin. The beads can then be washed and vacuum aspirated several times before analysis on a suspension array system using software provided with an instrument. The identity of antigens that can be used to selectively extract the exosomes can then be elucidated from the analysis.

Assays using imaging systems can be utilized to detect and quantify proteins expressed on the surface of an exosome in order to discover and specifically select for and enrich exosomes from specific tissue or tumor types. Antibodies, peptides or cells conjugated to multiple well multiplex carbon coated plates can be used. Simultaneous measurement of many analytes in a well can be achieved through the use of capture antibodies arrayed on the patterned carbon working surface. Analytes can then be detected with antibodies labeled with reagents in electrode wells with an enhanced electro-chemiluminescent plate. Any molecule of organic origin can be successfully conjugated to the carbon coated plate. Proteins expressed on the surface of exosomes can be identified from this assay and can be used as targets to specifically select for and enrich exosomes from specific tissue or tumor types. The binding agent can also be a novel aptamer. An aptamer for a target can be identified using systematic evolution of ligands by exponential enrichment (SELEX) (Tuerk & Gold, Science 249:505-510, 1990; Ellington & Szostak, Nature 346:818-822, 1990), such as described in U.S. Pat. No. 5,270,163. A library of nucleic acids can be contacted with a target exosome, and those nucleic acids specifically bound to the target are partitioned from the remainder of nucleic acids in the library which do not specifically bind the target. The partitioned nucleic acids are amplified to yield a ligand-enriched pool. Multiple cycles of binding, partitioning, and amplifying (i.e., selection) result in identification of one or more aptamers with the desired activity. Another method for identifying an aptamer to isolate exosomes is described in U.S. Pat. No. 6,376,19, which describes increasing or decreasing frequency of nucleic acids in a library by their binding to a chemically synthesized peptide. Modified methods, such as Laser SELEX or deSELEX as described in U.S. Patent Publication No. 20090264508 can also be used.

Microfluidics

The methods for isolating or identifying exosomes can be used in combination with microfluidic devices. The methods of isolating exosomes disclosed herein can be performed using microfluidic devices. Microfluidic devices, which may also be referred to as “lab-on-a-chip” systems, biomedical micro-electro-mechanical systems (bioMEMs), or multicomponent integrated systems, can be used for isolating, and analyzing, exosomes. Such systems miniaturize and compartmentalize processes that allow for binding of exosomes, detection of exosomal biomarkers, and other processes.

A microfluidic device can also be used for isolation of an exosome through size differential or affinity selection. For example, a microfluidic device can use one more channels for isolating an exosome from a biological sample based on size, or by using one or more binding agents for isolating an exosome, from a biological sample. A biological sample can be introduced into one or more microfluidic channels, which selectively allows the passage of exosomes. The selection can be based on a property of the exosomes, for example, size, shape, deformability, biomarker profile, or bio-signature.

Alternatively, a heterogeneous population of exosomes can be introduced into a microfluidic device, and one or more different homogeneous populations of exosomes can be obtained. For example, different channels can have different size selections or binding agents to select for different exosome populations. Thus, a microfluidic device can isolate a plurality of exosomes, wherein at least a subset of the plurality of exosomes comprises a different bio-signature from another subset of said plurality of exosomes. For example, the microfluidic device can isolate at least 2, 3, 4, 5, 6, 7, 8, 9, 10, 15, 20, 25, 30, 40, 50, 60, 70, 80, 90, or 100 different subsets of exosomes, wherein each subset of exosomes comprises a different bio-signature.

In some embodiments, the microfluidic device can comprise one or more channels that permit further enrichment or selection of exosomes. A population of exosomes that has been enriched after passage through a first channel can be introduced into a second channel, which allows the passage of the desired exosome population to be further enriched, such as through binding agents present in the second channel.

Array-based assays and bead-based assays can be used with microfluidic device. For example, the binding agent can be coupled to beads and the binding reaction between the beads and exosomes can be performed in a microfluidic device. Multiplexing can also be performed using a microfluidic device. Different compartments can comprise different binding agents for different populations of exosomes, where each population is of a different cell-of-origin specific exosome population or each population has a different bio-signature. The hybridization reaction between the microspheres and exosomes can be performed in a microfluidic device and the reaction mixture can be delivered to a detection device. The detection device, such as a dual or multiple laser detection system can be part of the microfluidic system and can use a laser to identify each bead or microsphere by its color-coding, and another laser can detect the hybridization signal associated with each bead.

Examples of microfluidic devices that may be used, or adapted for use with exosomes, include but are not limited to those described in U.S. Pat. Nos. 7,591,936, 7,581,429, 7,579,136, 7,575,722, 7,568,399, 7,552,741, 7,544,506, 7,541,578, 7,518,726, 7,488,596, 7,485,214, 7,467,928, 7,452,713, 7,452,509, 7,449,096, 7,431,887, 7,422,725, 7,422,669, 7,419,822, 7,419,639, 7,413,709, 7,411,184, 7,402,229, 7,390,463, 7,381,471, 7,357,864, 7,351,592, 7,351,380, 7,338,637, 7,329,391, 7,323,140, 7,261,824, 7,258,837, 7,253,003, 7,238,324, 7,238,255, 7,233,865, 7,229,538, 7,201,881, 7,195,986, 7,189,581, 7,189,580, 7,189,368, 7,141,978, 7,138,062, 7,135,147, 7,125,711, 7,118,910, and 7,118,661.

### Cell-of-Origin and Disease-Specific Exosomes

The bindings agents disclosed herein can be used to isolate a heterogeneous population of exosomes from a sample or can be used to isolate or identify a homogeneous population of exosomes, such as cell-of-origin specific exosomes or exosomes with specific bio-signatures. A homogeneous population of exosomes, such as cell-of-origin specific exosomes, can be analyzed and used to characterize a phenotype for a subject. Cell-of-origin specific exosomes are exosomes derived from specific cell types, which can include, but are not limited to, cells of a specific tissue, cells from a specific tumor of interest or a diseased tissue of interest, circulating tumor cells, or cells of maternal or fetal origin. The exosomes may be derived from tumor cells or lung, pancreas, stomach, intestine, bladder, kidney, ovary, testis, skin, colorectal, breast, prostate, brain, esophagus, liver, placenta, or fetal cells. The isolated exosomes can also be from a particular sample type, such as urinary exosomes.

Cell-of-origin specific exosomes from a biological sample can be isolated using one or more binding agents that are specific to a cell-of-origin. Exosomes for analysis of a disease or condition can be isolated using one or more binding agents specific for biomarkers for that disease or condition.

The exosomes can be concentrated prior to isolation of cell-of-origin specific exosomes, such as through centrifugation, chromatography, or filtration, as described above, to produce a heterogeneous population of exosomes prior to isolation of cell-of-origin specific exosomes. Alternatively, the exosomes are not concentrated, or the biological sample is not enriched for exosomes, prior to isolation of cell-of-origin exosomes.

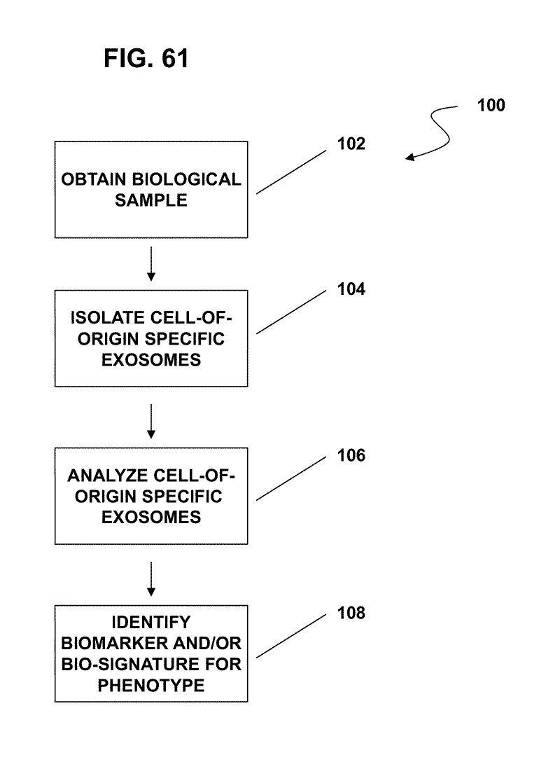

FIG. 61 illustrates a flowchart which depicts one method 100 for isolating or identifying cell-of-origin specific exosomes. First, a biological sample is obtained from a subject in step 102. The sample can be obtained from a third party or from the same party performing the analysis. Next, cell-of-origin specific exosomes are isolated from the biological sample in step 104. The isolated cell-of-origin specific exosomes are then analyzed in step 106 and a biomarker or bio-signature for a particular phenotype is identified in step 108. The method may be used for a number of phenotypes. In some embodiments, prior to step 104, exosomes are concentrated or isolated from a biological sample to product a heterogeneous population of exosomes. For example, heterogeneous population of exosomes may be isolated using centrifugation, chromatography, filtration, or other methods as described above, prior to use of one or more binding agents specific for isolating or identifying exosomes derived from specific cell types, or cell-of-origin specific exosomes.

Cell-of-origin specific exosomes can be isolated from a biological sample of a subject by employing one or more binding agents that bind with high specificity to the cell-of-origin specific exosomes. In some instances, a single binding agent can be employed to isolate cell-of-origin specific exosomes. In other instances, a combination of binding agents may be employed to isolate cell-of-origin specific exosomes. For example, at least 2, 3, 4, 5, 6, 7, 8, 9, 10, 11, 12, 13, 14, 15, 16, 17, 18, 19, 20, 25, 50, 75, or 100 different binding agents may be used to isolate cell-of-origin exosomes. Therefore, an exosome population (e.g., exosomes having the same binding agent profile) can be identified by utilizing a single or a plurality of binding agents.

One or more binding agents can be selected based on their specificity for a target antigen(s) that is specific to a cell-of-origin, tumor or disease. Non-limiting examples of antigens which may be used singularly, or in combination, to isolate a cell-of-origin specific exosome, disease specific exosome, or tumor specific exosome is shown in FIG. 1 and are also described below. The antigen may be membrane bound antigens which are accessible to binding agents. The antigen can also be a biomarker for the phenotype.

Breast Cancer

An exosome derived from a breast cancer cell can be isolated using a binding agent (e.g., antibody), that is specific for an antigen that is associated with a cell of breast cancer origin (e.g., cells of glandular or stromal origin). An exosome derived from a breast cancer cell can be isolated using an antigen including, but not limited to, BCA-225, hsp70, MART1, ER, VEGFA, Class III b-tubulin, HER2/neu (for Her2+BC), GPR30, ErbB4 (JM) isoform, MPR8, MISIIR, fragments thereof, any combination thereof, or any combination of antigens that are specific for a breast cancer cell.

Ovarian Cancer

An exosome derived from an ovarian cancer cell can be isolated using an antibody, or any other binding agent, for an antigen that is specific for a cell of ovarian cancer origin including, but not limited to, CA125, VEGFR2, HER2, MISIIR, VEGFA, CD24, fragments thereof, any combination thereof, or any combination of antigens that is specific for an ovarian cancer cell.

Lung Cancer

An exosome derived from a lung cancer cell can be isolated using an antibody, or any other binding agent, for an antigen that is specific for a cell of lung cancer origin including, but not limited to, CYFRA21-1, TPA-M, TPS, CEA, SCC-Ag, XAGE-1b, HLA Class 1, TA-MUC1, KRAS, hENT1, kinin B1 receptor, kinin B2 receptor, TSC403, HTI56, DC-LAMP, fragments thereof, any combination thereof, or any combination of antigens that is specific for a lung cancer cell.

Colon Cancer

An exosome derived from a colon cancer cell can be isolated using an antibody, or any other binding agent, for an antigen that is specific for a cell of colon cancer origin including, but not limited to, CEA, MUC2, GPA33, CEACAM5, ENFB1, CCSA-3, CCSA-4, ADAM10, CD44, NG2, ephrin B1, plakoglobin, galectin 4, RACK1, tetraspanin-8, FASL, A33, CEA, EGFR, dipeptidase 1, PTEN, Na(+)-dependent glucose transporter, UDP-glucuronosyltransferase 1A, fragments thereof, any combination thereof, or any combination of antigens that is specific for a colon cancer cell.

Prostate Cancer

An exosome derived from a prostate cancer cell can be isolated using an antibody, or any other binding agent, for an antigen that is specific for a cell of prostate cancer origin including, but not limited to, PSA, TMPRSS2, FASLG, TNFSF10, PSMA, NGEP, Il-7RI, CSCR4, CysLT1R, TRPM8, Kv1.3, TRPV6, TRPM8, PSGR, MISIIR, galectin-3, PCA3, TMPRSS2:ERG, fragments thereof, any combination thereof, or any combination of antigens that is specific for a prostate cancer cell.

Brain Cancer

An exosome derived from brain cancer cell can be isolated using an antibody, or any other binding agent, for an antigen that is specific for a cell of brain cancer origin including, but not limited to, PRMT8, BDNF, EGFR, DPPX, Elk, Densin-180, BAI2, BAI3, fragments thereof, any combination thereof, or any combination of antigens that is specific for a brain cancer cell.

Blood Cancer

An exosome derived from a hematological malignancy cell can be isolated using an antibody, or any other binding agent, for an antigen that is specific for a cell of hematological malignancy origin including, but not limited to, CD44, CD58, CD31, CD11a, CD49d, GARP, BTS, Raftlin, fragments thereof, any combination thereof, or any combination of antigens that is specific for a hematological malignancy cell.

Melanoma

An exosome derived from a melanoma cell can be isolated using an antibody, or any other binding agent, for an antigen that is specific for a cell of melanoma origin including, but not limited to, DUSP1, TYRP1, SILV, MLANA, MCAM, CD63, Alix, hsp70, meosin, p120 catenin, PGRL, syntaxin binding protein 1 & 2, caveolin, fragments thereof, any combination thereof, or any combination of antigens that is specific for a melanoma cell.

Liver Cancer

An exosome derived from a hepatocellular carcinoma cell can be isolated using an antibody, or any other binding agent, for an antigen that is specific for a cell of hepatocellular carcinoma origin including, but not limited to, HBxAg, HBsAg, NLT, fragments thereof, any combination thereof, or any combination of antigens that is specific for a hepatocellular carcinoma cell.

Cervical Cancer

An exosome derived from a cervical cancer cell can be isolated using an antibody, or any other binding agent, for an antigen that is specific for a cell of cervical cancer origin including, but not limited to, MCT-1, MCT-2, MCT-4, fragments thereof, any combination thereof, or any combination of antigens that is specific for a cervical cancer cell.

Endometrial Cancer

An exosome derived from an endometrial cancer cell can be isolated using an antibody, or any other binding agent, for an antigen that is specific for a cell of endometrial cancer origin including, but not limited to, Alpha V Beta 6 integrin, fragments thereof, any combination thereof, or any combination of antigens that is specific for an endometrial cancer cell.

Psoriasis

An exosome for characterizing psoriasis can be isolated using an antibody, or any other binding agent, for an antigen that is specific for psoriasis including, but not limited to, flt-1, VPF receptors, kdr, fragments thereof, any combination thereof, or any combination of antigens that is specific to psoriasis.

Autoimmune Disease

An exosome for characterizing an autoimmune disease can be isolated using an antibody, or any other binding agent, for an antigen that is specific for an autoimmune disease including, but not limited to, Tim-2, fragments thereof, or any combination of antigens that is specific to an autoimmune disease.

Irritable Bowel Disease

An exosome for characterizing irritable bowel disease (IBD) or syndrome (IBS) can be isolated using an antibody, or any other binding agent, for an antigen that is specific for IBD or IBS including, but not limited to, IL-16, IL-1beta, IL-12, TNF-alpha, interferon-gamma, IL-6, Rantes, II-12, MCP-1, 5HT, fragments thereof, or any combination of antigens that is specific to IBD or IBS.

Diabetes

An exosome derived from a pancreatic cell can be isolated using an antibody, or any other binding agent, for an antigen that is specific for a cell of pancreatic origin including, but not limited to, IL-6, CRP, RBP4, fragments thereof, any combination thereof, or any combination of antigens that is specific for a pancreatic cell.

Barrett's Esophagus

An exosome for characterizing Barrett's Esophagus can be isolated using an antibody, or any other binding agent, for an antigen that is specific for Barrett's Esophagus including, but not limited to, p53, MUC1, MUC6, fragments thereof, any combination thereof, or any combination of antigens that is specific to Barrett's Esophagus.

Fibromyalgia

An exosome for characterizing fibromyalgia can be isolated using an antibody, or any other binding agent, for an antigen that is specific for fibromyalgia including, but not limited to, neopterin, gp130, fragments thereof, any combination thereof, or any combination of antigens that is specific to fibromyalgia.

Prostatic Hyperplasia

An exosome derived from a benign prostatic hyperplasia (BPH) cell can be isolated using an antibody, or any other binding agent, for an antigen that is specific for a cell of BPH origin including, but not limited to, KIA1, intact fibronectin, fragments thereof, any combination thereof, or any combination of antigens that is specific for a BPH cell.

Multiple Sclerosis

An exosome for characterizing multiple sclerosis (MS) can be isolated using an antibody, or any other binding agent, for an antigen that is specific for MS including, but not limited to, B7, B7-2, CD-95 (fas), Apo-1/Fas, fragments thereof, any combination thereof, or any combination of antigens that is specific to MS.

Parkinson's Disease

An exosome for characterizing Parkinson's disease can be isolated using an antibody, or any other binding agent, for an antigen that is specific for Parkinson's disease including, but not limited to, PARK2, ceruloplasmin, VDBP, tau, DJ-1, fragments thereof, any combination thereof, or any combination of antigens that is specific to Parkinson's disease.

Rheumatic Disease

An exosome for characterizing rheumatic disease can be isolated using an antibody, or any other binding agent, for an antigen that is specific for rheumatic disease including, but not limited to, Citrulinated fibrin a-chain, CD5 antigen-like fibrinogen fragment D, CD5 antigen-like fibrinogen fragment B, TNF alpha, fragments thereof, any combination thereof, or any combination of antigens that is specific to rheumatic disease.

Alzheimer's Disease

An exosome derived from a neuron of a patient suffering from Alzheimer's disease can be further isolated using an antibody, or any other binding agent, for an antigen that including, but not limited to, APP695, APP751 or APP770, BACE1, cystatin C, amyloid β, T-tau, complement factor H or alpha-2-macroglobulin, fragments thereof, any combination thereof, or any combination of antigens that are specific for Alzheimer's.

Head and Neck Cancer

An exosome derived from a head and neck cancer cell can be isolated using an antibody, or any other binding agent, for an antigen that is specific for a cell of head and neck cancer origin including, but not limited to, EGFR, EphB4 or Ephrin B2, fragments thereof, any combination thereof, or any combination of antigens that is specific for a head and neck cancer cell.

Gastrointestinal Stromal Tumor

An exosome derived from a gastrointestinal stromal tumor (GIST) cell can be isolated using an antibody, or any other binding agent, for an antigen that is specific for a cell of GIST origin including, but not limited to, c-kit PDGFRA, NHE-3, fragments thereof, any combination thereof, or any combination of antigens that is specific for a GIST cell.

Renal Cell Carcinoma

An exosome derived from a renal cell carcinomas (RCC) cell can be isolated using an antibody, or any other binding agent, for an antigen that is specific for a cell of RCC origin including, but not limited to, c PDGFRA, VEGF, HIF 1 alpha, fragments thereof, any combination thereof, or any combination of antigens that is specific for a RCC cell.

Schizophrenia

An exosome for characterizing schizophrenia can be isolated using an antibody, or any other binding agent, for an antigen that is specific for schizophrenia including, but not limited to, ATP5B, ATP5H, ATP6V1B, DNM1, fragments thereof, any combination thereof, or any combination of antigens that is specific to schizophrenia.

Peripheral Neuropathic Pain

An exosome derived from a nerve cell of a patient suffering from peripheral neuropathic pain can be isolated using an antibody, or any other binding agent, for an antigen that is specific for peripheral neuropathic pain including, but not limited to, OX42, ED9, fragments thereof, any combination thereof, or any combination of antigens that is specific for peripheral neuropathic pain.

Chronic Neuropathic Pain

An exosome derived from a nerve cell of a patient suffering from chronic neuropathic pain can be isolated using an antibody, or any other binding agent, for an antigen that is specific for chronic neuropathic pain including, but not limited to, chemokine receptor (CCR2/4), fragments thereof, or any combination of antigens that is specific for chronic neuropathic pain.

Prion Disease

An exosome derived from a cell of a patient suffering from prion disease can be isolated using an antibody, or any other binding agent, for an antigen that is specific for prion disease including, but not limited to, PrPSc, 14-3-3 zeta, S-100, AQP4, fragments thereof, or any combination of antigens that is specific for prion disease.

Stroke

An exosome for characterizing stroke can be isolated using an antibody, or any other binding agent, for an antigen that is specific for stroke including, but not limited to, S-100, neuron specific enolase, PARK7, NDKA, ApoC-I, ApoC-III, SAA or AT-III fragment, Lp-PLA2, hs-CRP, fragments thereof, any combination thereof, or any combination of antigens that is specific to stroke.

Cardiovascular Disease

An exosome for characterizing a cardiovascular disease can be isolated using an antibody, or any other binding agent, for an antigen that is specific for a cardiovascular disease including, but not limited to, FATP6, fragments thereof, or any combination of antigens that is specific to a cardiovascular disease or cardiac cell.

Esophageal Cancer

An exosome derived from an esophageal cancer cell can be isolated using an antibody, or any other binding agent, for an antigen that is specific for a cell of esophageal cancer origin including, but not limited to, CaSR, fragments thereof, or any combination of antigens that is specific for an esophageal cancer cell.

Tuberculosis

An exosome for characterizing tuberculosis (TB) can be isolated using an antibody, or any other binding agent, for an antigen that is specific for TB including, but not limited to, antigen 60, HSP, Lipoarabinomannan, Sulfolipid, antigen of acylated trehalose family, DAT, TAT, Trehalose 6,6-dimycolate (cord-factor) antigen, fragments thereof, any combination thereof, or any combination of antigens that is specific to TB.

HIV

An exosome for characterizing HIV can be isolated using an antibody, or any other binding agent, for an antigen that is specific for HIV including, but not limited to, gp41, gp120, fragments thereof, any combination thereof, or any combination of antigens that is specific to HIV.

Autism

An exosome for characterizing autism can be isolated using an antibody, or any other binding agent, for an antigen that is specific for autism including, but not limited to, VIP, PACAP, CGRP, NT3, fragments thereof, any combination thereof, or any combination of antigens that is specific to autism.

Asthma

An exosome for characterizing asthma can be isolated using an antibody, or any other binding agent, for an antigen that is specific for asthma including, but not limited to, YKL-40, S-nitrosothiols, SSCA2, PAI, amphiregulin, periostin, fragments thereof, any combination thereof, or any combination of antigens that is specific to asthma.

Lupus

An exosome for characterizing lupus can be isolated using an antibody, or any other binding agent, for an antigen that is specific for lupus including, but not limited to, TNFR, fragments thereof, or any combination of antigens that is specific to lupus.

Cirrhosis

An exosome for characterizing cirrhosis can be isolated using an antibody, or any other binding agent, for an antigen that is specific for cirrhosis including, but not limited to, NLT, HBsAg, fragments thereof, any combination thereof, or any combination of antigens that is specific to cirrhosis.

Influenza

An exosome for characterizing influenza can be isolated using an antibody, or any other binding agent, for an antigen that is specific for influenza including, but not limited to, hemagglutinin, neurominidase, fragments thereof, any combination thereof, or any combination of antigens that is specific to influenza.

Vulnerable Plaque

An exosome for characterizing vulnerable plaque can be isolated using an antibody, or any other binding agent, for an antigen that is specific for vulnerable plaque including, but not limited to, Alpha v. Beta 3 integrin, MMP9, fragments thereof, any combination thereof, or any combination of antigens that is specific to vulnerable plaque.

A cell-of-origin specific exosome may be isolated using novel binding agents, using methods as described above. Furthermore, a cell-of-origin specific exosome can also be isolated from a biological sample using isolation methods based on cellular binding partners or binding agents of such exosomes. Such cellular binding partners can include but are not limited to peptides, proteins, RNA, DNA, apatmers, cells or serum-associated proteins that only bind to such exosomes when one or more specific biomarkers are present. Isolation of a cell-of-origin specific exosome can be carried out with a single binding partner or binding agent, or a combination of binding partners or binding agents whose singular application or combined application results in cell-of-origin specific isolation. Non-limiting examples of such binding agents are provided in FIG. 2. For example, an exosome for characterizing breast cancer can be isolated with one or more binding agents including, but not limited to, estrogen, progesterone, Herceptin (Trastuzumab), CCND1, MYC PNA, IGF-1 PNA, MYC PNA, SC4 aptamer (Ku), AII-7 aptamer (ERB2), Galectin-3, mucin-type 0-glycans, L-PHA, Galectin-9, or any combination thereof.

A binding agent may also be used for isolating the cell-of-origin specific exosome based on i) the presence of antigens specific for cell-of-origin specific exosomes cells, ii) the absence of markers specific for cell-of-origin specific exosomes, or iii) expression levels of biomarkers specific for cell-of-origin specific exosomes. A heterogeneous population of exosomes is applied to a surface coated with specific binding agents designed to rule out or identify the cell-of-origin characteristics of the exosomes. Various binding agents, such as antibodies, can be arrayed on a solid surface or substrate and the heterogeneous population of exosomes is allowed to contact the solid surface or substrate for a sufficient time to allow interactions to take place. Specific binding or non-binding to given antibody locations on the array surface or substrate can then serve to identify antigen specific characteristics of the exosome population that are specific to a given cell-of-origin.

A cell-of-origin specific exosome can be enriched or isolated using one or more binding agents using a magnetic capture method, fluorescence activated cell sorting or laser cytometry as described above. Magnetic capture methods can include, but are not limited to, the use of magnetically activated cell sorter (MACS) microbeads or magnetic columns. Examples of immunoaffinity and magnetic particle methods that can be used is described in U.S. Pat. No. 4,551,435, 4,795,698, 4,925,788, 5,108,933, 5,186,827, 5,200,084 or 5,158,871. A cell-of-origin specific exosome can also be isolated following the general methods described in U.S. Pat. No. 7,399,632, by using combination of antigens specific to an exosome.

Any other method for isolating or otherwise enriching the cell-of-origin specific exosomes with respect to a biological sample may also be used in combination with the present invention. For example, size exclusion chromatography such as gel permeation columns, centrifugation or density gradient centrifugation, and filtration methods can be used in combination with the antigen selection methods described herein. The cell-of-origin specific exosomes may also be isolated following the methods described in Koga et al., Anticancer Research, 25:3703-3708 (2005), Taylor et al., Gynecologic Oncology, 110:13-21 (2008), Nanjee et al., Clin Chem, 2000; 46:207-223 or U.S. Pat. No. 7,232,653.

Exosome Assessment

A phenotype can be characterized for a subject by analyzing a biological sample from the subject and determining the level, amount, or concentration of one or more populations of exosomes in the sample. An exosome can be purified or concentrated prior to determining the amount of exosomes. Alternatively, the amount of exosomes can be directly assayed from a sample, without prior purification or concentration. The exosomes can be cell-of-origin specific exosomes or exosomes with a specific biomarker or combination of biomarkers. The amount of exosomes can be used to characterize a phenotype, such as a diagnosis, theranosis or prognosis of a condition or disease. The amount may be used to determine a physiological or biological state, such as pregnancy or the stage of pregnancy. The amount of exosomes can also be used to determine treatment efficacy, stage of a disease or condition, or progression of a disease or condition. For example, the amount of exosomes can be proportional to an increase in disease stage or progression.

The exosomes can be evaluated by comparing the level of exosomes with a reference level or value of exosomes. The reference value can be particular to physical or temporal endpoint. For example, the reference value can be from the same subject from whom a sample is assessed for an exosome, or the reference value can be from a representative population of samples (e.g., samples from normal subjects not exhibiting a symptom of disease). Therefore, a reference value can provide a threshold measurement which is compared to a subject sample's readout for one or more exosome populations assayed in a given sample. Such reference values may be set according to data pooled from groups of sample corresponding to a particular cohort, including but not limited to age (e.g., newborns, infants, adolescents, young, middle-aged adults, seniors and adults of varied ages), racial/ethnic groups, normal versus diseased subjects, smoker v. non-smoker, subject receiving therapy versus untreated subject, different time points of treatment for a particular individual or group of subjects similarly diagnosed or treated or combinations thereof.

A reference value may be based on samples assessed from the same subject so to provide individualized tracking. Frequent testing of a patient may provide better comparisons to the reference values previously established for a particular patient and would allow a physician to more accurately assess the patient's disease stage or progression, and to inform a better decision for treatment. The reduced intraindividual variance of exosomes levels would allow a more specific and individualized threshold to be defined for the patient. Temporal intrasubject variation allows each individual to serve as a longitudinal control for optimum analysis of disease or physiological state.

Reference values can be established for unaffected individuals (of varying ages, ethnic backgrounds and sexes) without a particular phenotype by determining the amount of exosomes in an unaffected individual. For example, a reference value for a reference population can be utilized as a baseline for detection of one or more exosome populations in a test subject. If a sample from a subject has a level or value that is similar to the reference, the subject can be identified to not have the disease, or of having a low likelihood of developing a disease.

Alternatively, reference values or levels can be established for individuals with a particular phenotype by determining the amount of one or more populations of exosomes in an individual with the phenotype. In addition, an index of values can be generated for a particular phenotype. For example, different disease stages can have different values, such as obtained from individuals with the different disease stages. A subject's value can be compared to the index and a diagnosis or prognosis of the disease can be determined, such as the disease stage or progression. In other embodiments, an index of values is generated for therapeutic efficacies. For example, the level of exosomes of individuals with a particular disease can be generated and noted what treatments were effective for the individual. The levels can be used to generate values of which is a subject's value is compared, and a treatment or therapy can be selected for the individual.

For example, a reference value can be determined for individuals unaffected with a particular cancer, by isolating exosomes with an antigen that specifically targets for the particular cancer. For example, individuals with varying stages of colorectal cancer and noncancerous polyps can be surveyed using the same techniques described for unaffected individuals and the levels of circulating exosomes for each group defined as means±standard deviations from at least two separate experiments performed in triplicate. Comparisons between these groups can be made using statistical applications such as one-way ANOVA, followed by Tukey's multiple comparisons post-test comparing each population.

Reference values can also be established for disease recurrence monitoring (or exacerbation phase in MS), or for therapeutic response monitoring.

The values can be a quantitative or qualitative value. The values can be a direct measurement of the level of exosomes (example, mass per volume), or an indirect measure, such as the amount of a specific exosomal marker. The values can be a quantitative, such as a numerical value. In other embodiments, the value is qualitative, such as no exosomes, low level of exosomes, medium level, or high level of exosomes, or variations thereof.

The reference values can be stored in a database and used as a reference for the diagnosis, prognosis, or theranosis of a disease or condition based on the level or amount of exosomes, such as total amount of exosomes, or the amount of a specific population of exosomes, such as cell-of-origin specific exosomes or exosomes with one or more specific biomarkers.

Exosome levels may be characterized using mass spectrometry or flow cytometry. Analysis may also be carried out on exosomes by immunocytochemical staining, Western blotting, electrophoresis, chromatography or x-ray crystallography in accordance with procedures well known in the art. Exosomes may be characterized and quantitatively measured using flow cytometry as described in Clayton et al., Journal of Immunological Methods 2001; 163-174, which is herein incorporated by reference in its entirety. Exosome levels may be determined using binding agents as described above. For example, a binding agent to exosomes can be labeled and the label detected and used to determine the amount of exosomes in a sample. The binding agent can be bound to a substrate, such as arrays or particles, such as described above. Alternatively, the exosomes may be labeled directly.

Electrophoretic tags or eTags can also be used to determine the amount of exosomes. eTags are small fluorescent molecules linked to nucleic acids or antibodies and are designed to bind one specific nucleic acid sequence or protein, respectively. After the eTag binds its target, an enzyme is used to cleave the bound eTag from the target. The signal generated from the released eTag, called a “reporter,” is proportional to the amount of target nucleic acid or protein in the sample. The eTag reporters can be identified by capillary electrophoresis. The unique charge-to-mass ratio of each eTag reporter—that is, its electrical charge divided by its molecular weight—makes it show up as a specific peak on the capillary electrophoresis readout Thus by targeting a specific biomarker of an exosome with an eTag, the amount or level of exosomes can be determined

The exosome levels can determined from a heterogeneous population of exosomes, such as the total population of exosomes in a sample. Alternatively, the exosomes level is determined from a homogenous population, or substantially homogenous population of exosomes, such as the level of specific cell-of-origin exosomes, such as exosomes from prostate cancer cells. In yet other embodiments, the level is determined for exosomes with a particular biomarker or combination of biomarkers, such as a biomarker specific for prostate cancer. Determining the level of exosome can be performed in conjunction with determining the biomarker or combination of biomarkers of an exosome. Alternatively, determining the amount of exosome may be performed prior to or subsequent to determining the biomarker or combination of biomarkers of the exosomes.

Determining the amount of exosomes can be assayed in a multiplexed manner. For example, determining the amount of more than one population of exosomes, such as different cell-of-origin specific exosomes or exosomes with different biomarkers or combination of biomarkers, can be performed, such as those disclosed herein.

Specificity and Sensitivity

The level of exosomes as determined using one or more processes disclosed herein can be used to characterize a phenotype with increased sensitivity and the specificity. The sensitivity can be determined by: (number of true positives)/(number of true positives+number of false negatives). The specificity can be determined by: (number of true negatives)/(number of true negatives+number of false positives).

The level of exosomes as determined using one or more processes disclosed herein can be used to characterize a phenotype with at least 60, 61, 62, 63, 64, 65, 66, 67, 68, 69, or 70% sensitivity, such as with at least 71, 72, 73, 74, 75, 76, 77, 78, 79, 80, 81, 82, 83, 84, 85, 86, or 87% sensitivity. For example, the phenotype can be characterized with at least 87.1, 87.2, 87.3, 87.4, 87.5, 87.6, 87.7, 87.8, 87.9, 88.0, or 89% sensitivity, such as with at least 90% sensitivity. The phenotype can be characterized with at least 91, 92, 93, 94, 95, 96, 97, 98, 99 or 100% sensitivity.

The phenotype of a subject can also be characterized with at least 70, 71, 72, 73, 74, 75, 76, 77, 78, 79, 80, 81, 82, 83, 84, 85, 86, 87, 88, 89, 90, 91, 92, 93, 94, 95, 96, or 97% specificity, such as with at least 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9 or 100% specificity.

The phenotype can also be characterized with at least 70% sensitivity and at least 80, 90, 95, 99, or 100% specificity; at least 75% sensitivity and at least 80, 90, 95, 99, or 100% specificity; at least 80% sensitivity and at least 80, 85, 90, 95, 99, or 100% specificity; at least 85% sensitivity and at least 80, 85, 90, 95, 99, or 100% specificity; at least 86% sensitivity and at least 80, 85, 90, 95, 99, or 100% specificity; at least 87% sensitivity and at least 80, 85, 90, 95, 99, or 100% specificity; at least 88% sensitivity and at least 80, 85, 90, 95, 99, or 100% specificity; at least 89% sensitivity and at least 80, 85, 90, 95, 99, or 100% specificity; at least 90% sensitivity and at least 80, 85, 90, 95, 99, or 100% specificity; at least 95% sensitivity and at least 80, 85, 90, 95, 99, or 100% specificity; at least 99% sensitivity and at least 80, 85, 90, 95, 99, or 100% specificity; or at least 100% sensitivity and at least 80, 85, 90, 95, 99, or 100% specificity.

Furthermore, the confidence level for determining the specificity, sensitivity, or both, may be with at least 90, 91, 92, 93, 94, 95, 96, 97, 98, or 99% confidence.

### Bio-Signatures

A bio-signature of an exosome from a subject can be used to characterize a phenotype. A bio-signature can reflect the particular antigens or biomarkers that are present on an exosome. In addition, a bio-signature can also reflect one or more biomarkers that are carried in an exosome. Alternatively, a bio-signature can comprise a combination of one or more antigens or biomarkers that are present on an exosome with one or more biomarkers that are detected in the exosome.

The exosome can be purified or concentrated prior to determining the bio-signature of the exosome. Alternatively, the bio-signature of the exosome can be directly assayed from a sample, without prior purification or concentration. An exosome can also be isolated prior to assaying. For example, a cell-of-origin specific exosome can be isolated and its bio-signature determined. The bio-signature is used to determine a diagnosis, prognosis, or theranosis of a disease or condition. Therefore, a bio-signature can also be used to determine treatment efficacy, stage of a disease or condition, or progression of a disease or condition. Furthermore, a bio-signature may be used to determine a physiological state, such as pregnancy.

An exosomal characteristic in and of itself can be assessed to determine a bio-signature. The exosomal characteristic can be used to diagnose, detect or determine a disease stage or progression, the therapeutic implications of a disease or condition, or characterize a physiological state. An exosomal characteristic can include, but is not limited to, the level or amount of exosomes, temporal evaluation of the variation in exosomal half-life, circulating exosomal half-life or exosomal metabolic half-life, or the activity of an exosome.

In addition, a bio-signature can also correspond to an expression level, presence, absence, mutation, variant, copy number variation, truncation, duplication, modification, or molecular association of one or more biomarkers. A biomarker may be any exosomal component and can form its own signature. For example, the biomarker may be the RNA content of the exosome, such that the RNA signature includes one or more RNA species, such as, but not limited to, mRNA, miRNA, snoRNA, snRNA, rRNAs, tRNAs, siRNA, hnRNA, shRNA, or a combination thereof. Therefore, an exosome can be assayed to determine a RNA signature.

Other biomarkers include, but are not limited to, one or more proteins or peptides (e.g., providing a protein signature), nucleic acids (e.g. RNA signature as described, or a DNA signature), lipids (e.g. lipid signature), or combinations thereof. In some embodiments, the bio-signature can also comprise the type or amount of drug or drug metabolite present in an exosome (e.g. drug signature), as such drug may be taken by a subject from which the biological sample is obtained from, resulting in an exosome carrying such drug, or metabolites of such drug.

An RNA signature or DNA signature can also include a mutational, epigenetic modification, or genetic variant analysis of the RNA or DNA present in the exosome. In addition, a protein signature can include, but is not limited to, the mutation, modification, overexpression, underexpression, presence or absence of antigens, peptides, proteins or combinations thereof.

A bio-signature of an exosome can comprise one or more miRNA signatures combined with one or more additional signatures including, but not limited to, an mRNA signature, DNA signature, protein signature, peptide signature, antigen signature, or any combination thereof. For example, the bio-signature can comprise one or more miRNA biomarkers with one or more DNA biomarkers, one or more mRNA biomarkers, one or more snoRNA biomarkers, one or more protein biomarkers, one or more peptide biomarkers, one or more antigen biomarkers, one or more antigen biomarkers, one or more lipid biomarkers, or any combination thereof.

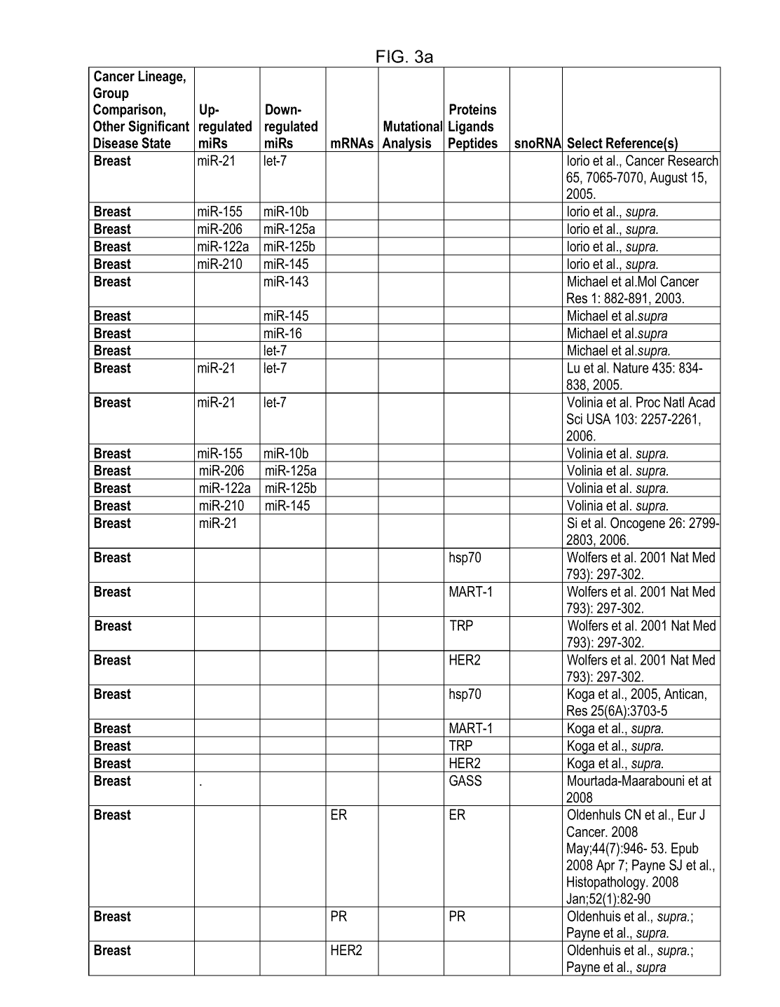

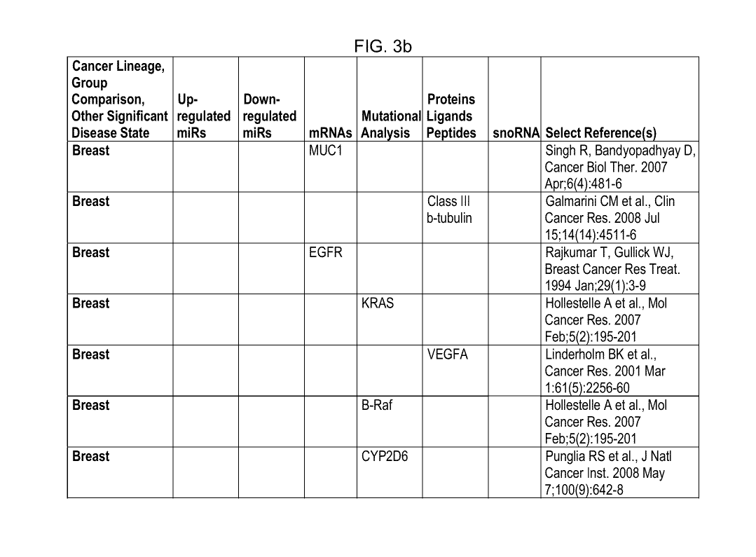

A bio-signature can comprise a combination of one or more antigens or binding agents (such as ability to bind one or more binding agents), such as listed in FIGS. 1 and 2, respectively. The bio-signature can further comprise one or more other biomarkers, such as, but not limited to, miRNA, DNA (e.g. single stranded DNA, complementary DNA, or noncoding DNA), or mRNA. For example, the bio-signature of an exosome can comprise a combination of one or more antigens, such as shown in FIG. 1, one or more binding agents, such as shown in FIG. 2, and one or more biomarkers for a condition or disease, such as listed in FIGS. 3-60. The bio-signature can comprise one or more biomarkers, for example miRNA, with one or more antigens specific for a cancer cell (for example, as shown in FIG. 1).

An exosome can have a bio-signature that is specific to the cell-of-origin and, as such, can be utilized to derive disease-specific or biological state specific diagnostic, prognostic or therapy-related bio-signatures representative of the cell-of-origin. An exosome may also have a bio-signature that is specific to a given disease or physiological condition that may be different from the bio-signature of the cell-of-origin, but no less important to the diagnosis, prognosis, staging, therapy-related determinations or physiological state characterization.

The bio-signature of an exosome, such as a cell-of-origin specific exosome described herein, can be used clinically in making decisions concerning treatment modalities, including therapeutic intervention, diagnostic criteria such as disease staging, disease monitoring, and disease stratification, and surveillance for detection, metastasis or recurrence or progression of disease. The bio-signature of an exosome, such as an isolated cell-of-origin specific exosome can further be used clinically to make treatment decisions, including whether to perform surgery or what treatment standards should be utilized along with surgery (e.g., either pre-surgery or post-surgery).

An exosome bio-signature can also be used in therapy related diagnostics to provide tests useful to diagnose a disease or choose the correct treatment regimen, as well as monitor a subject's response. Therapy related tests are useful to predict and assess drug response in individual subjects, i.e., to provide personalized medicine. Therapy related tests are also useful to select a subject for treatment who is particularly likely to benefit from the treatment or to provide an early and objective indication of treatment efficacy in an individual subject. For example, a treatment can be altered without the great expense of delaying beneficial treatment as well as the great financial cost of administering an ineffective drug(s).

Therapy related diagnostics are also useful in clinical diagnosis and management of a variety of diseases and disorders, which include, but are not limited to cardiovascular disease, cancer, infectious diseases, sepsis, neurological diseases, central nervous system related diseases, endovascular related diseases, and autoimmune related diseases or the prediction of drug toxicity, drug resistance or drug response. Therapy related tests may be developed in any suitable diagnostic testing format, which include, but are not limited to, e.g., immunohistochemical tests, clinical chemistry, immunoassay, cell-based technologies, nucleic acid tests or body imaging methods. Therapy related tests can further include but are not limited to, testing that aids in the determination of therapy, testing that monitors for therapeutic toxicity, or response to therapy testing. For example, a bio-signature can determine whether a particular disease or condition is resistant to a drug, and therefore, a physician need not waste valuable time with hit-and-miss treatment. Instead, to obtain early validation of a drug choice or treatment regimen, a bio-signature is determined for an exosome obtained from a subject, which then determines whether the particular subject's disease has the biomarker associated with drug resistance. Therefore, such a determination enables doctors to devote critical time as well as the patient's financial resources to effective treatments.

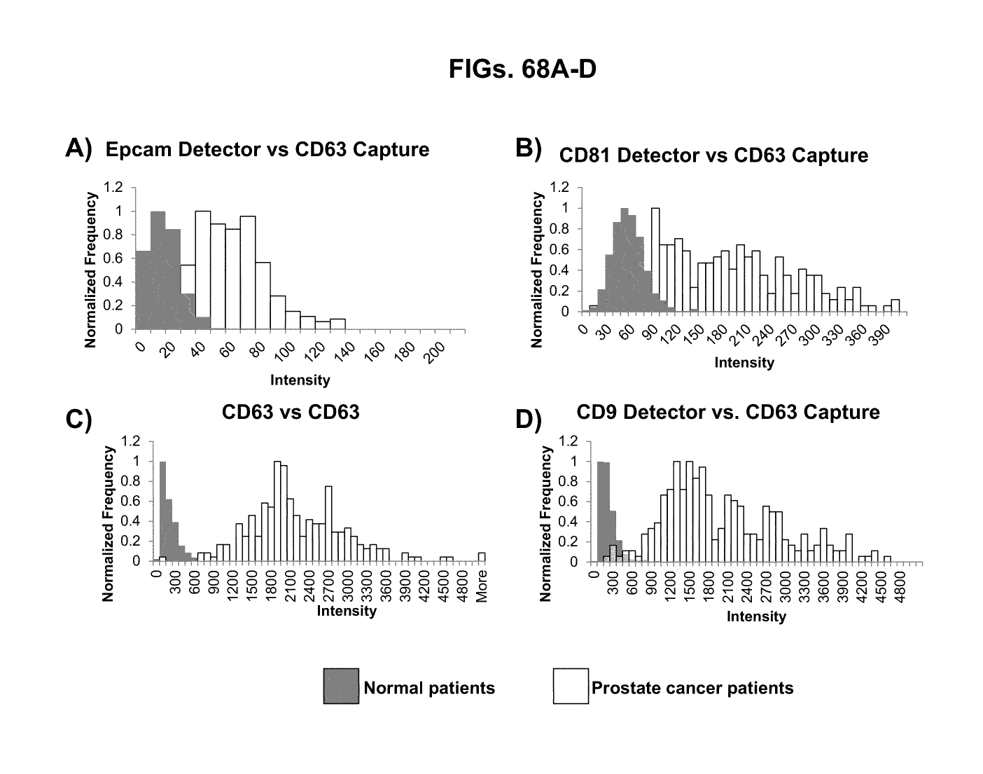

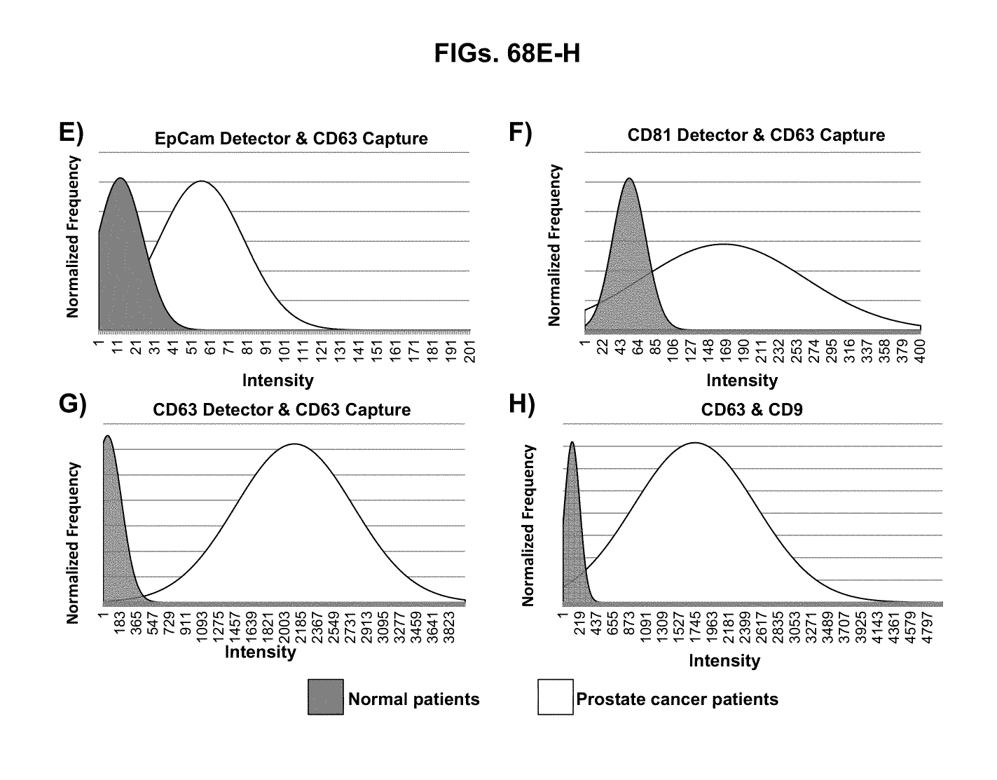

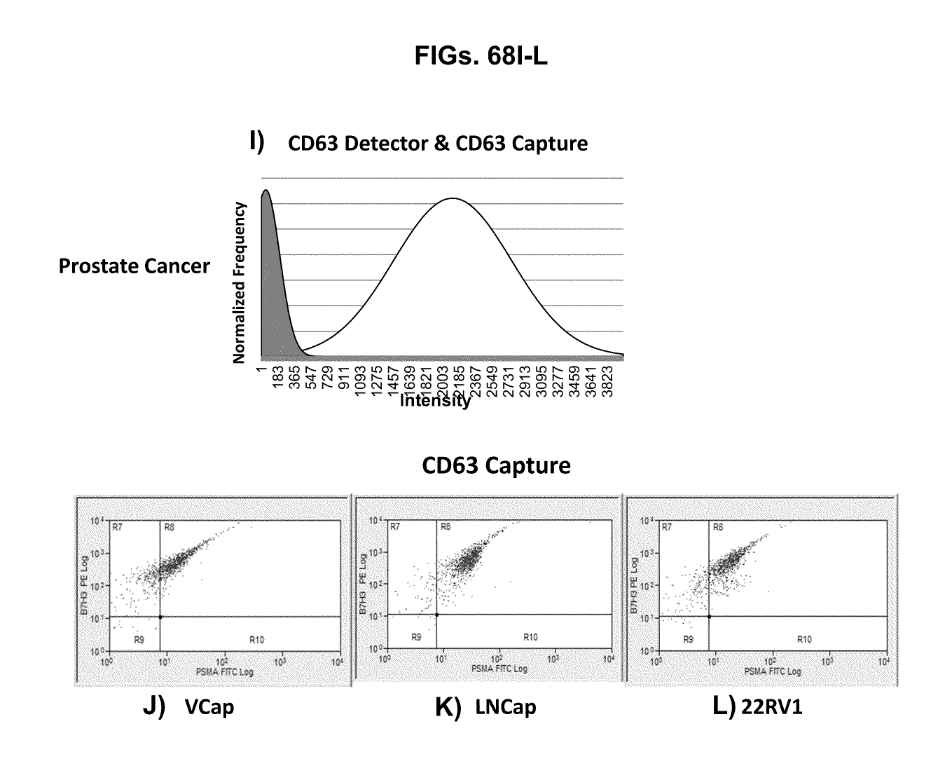

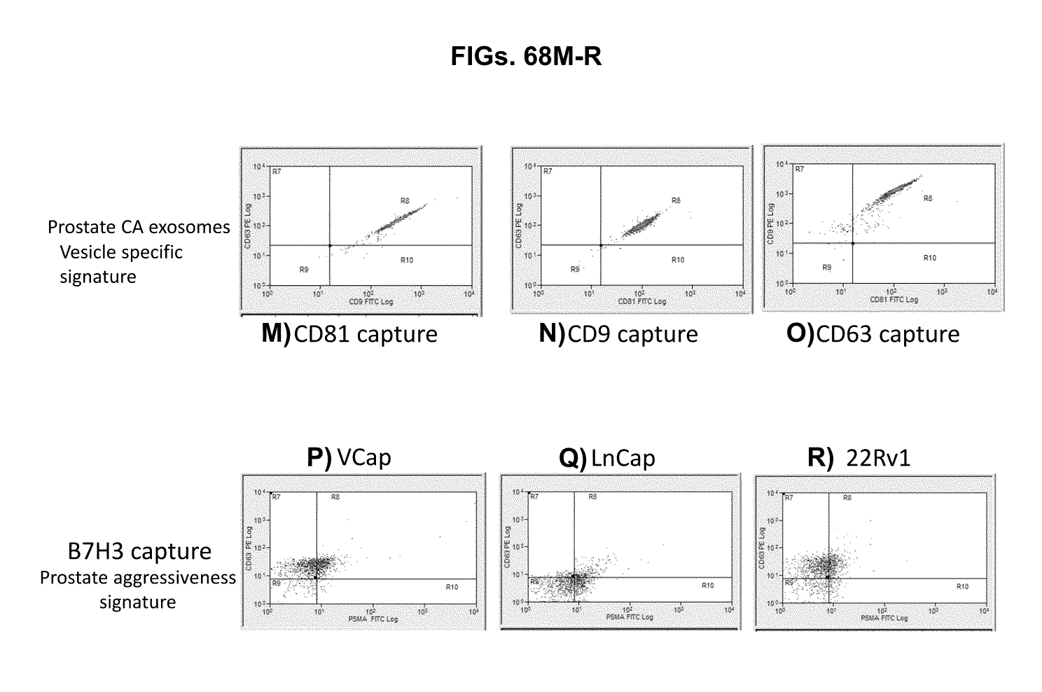

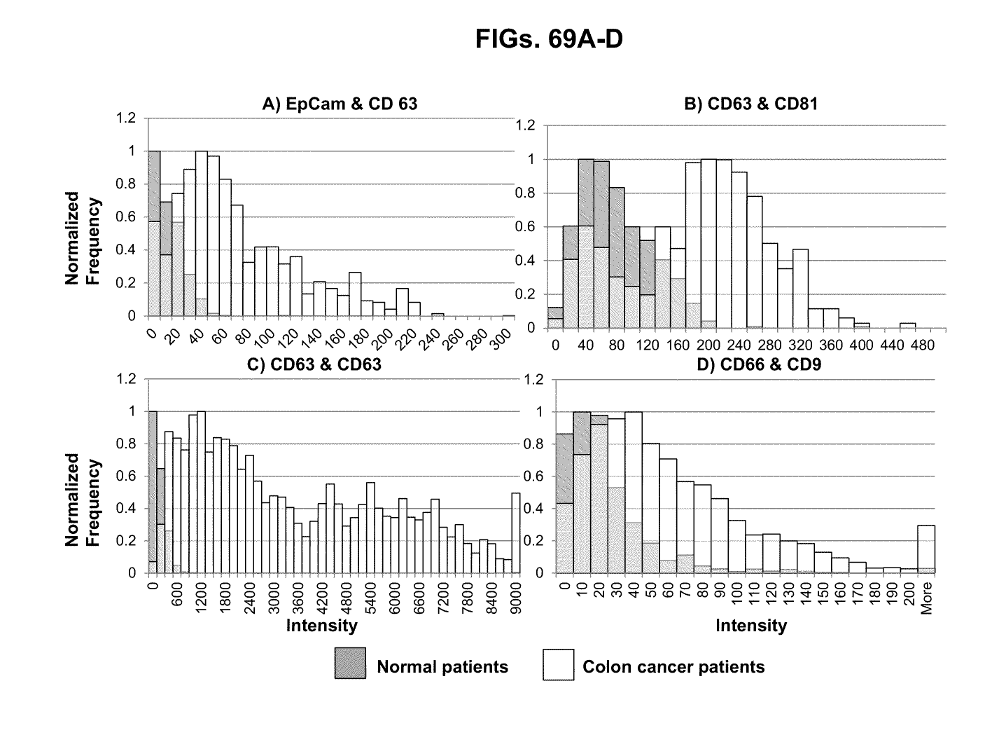

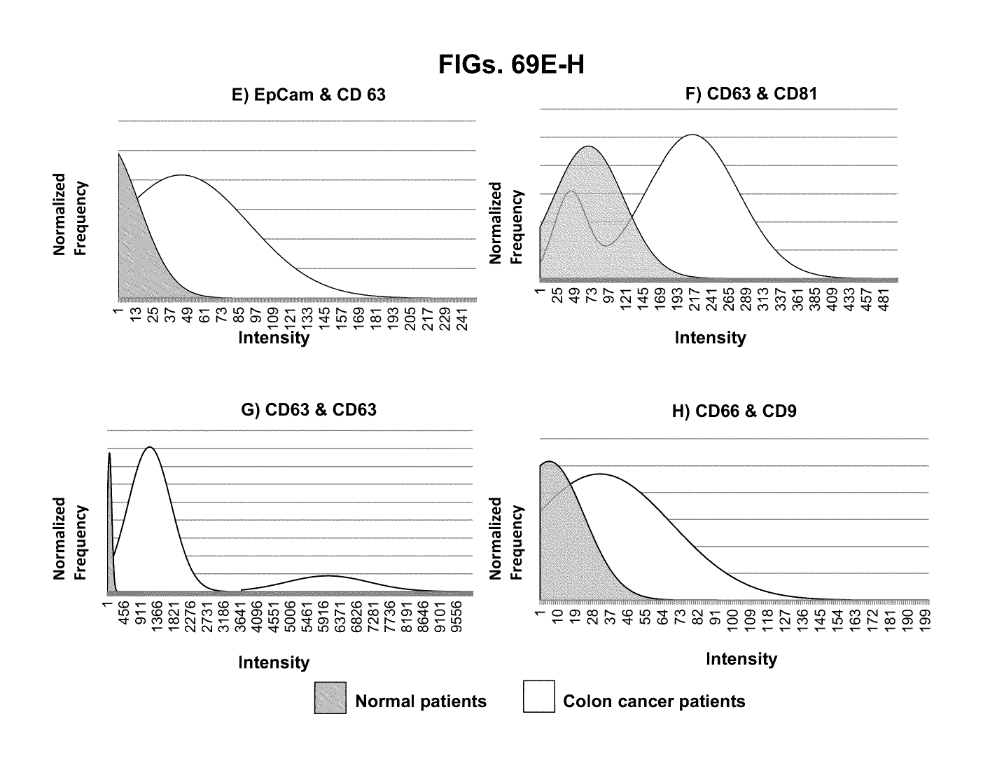

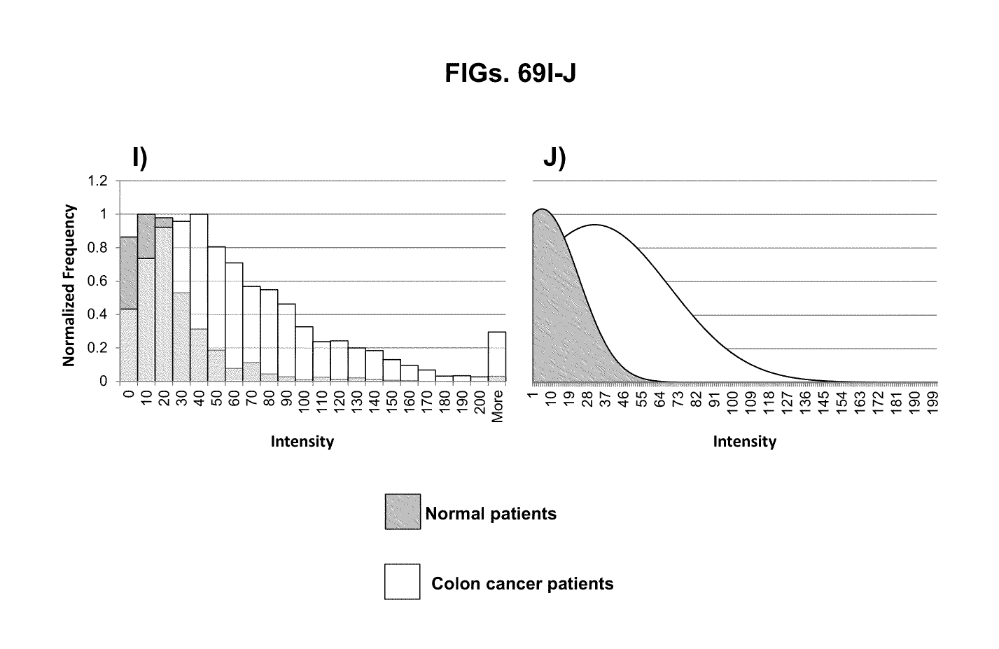

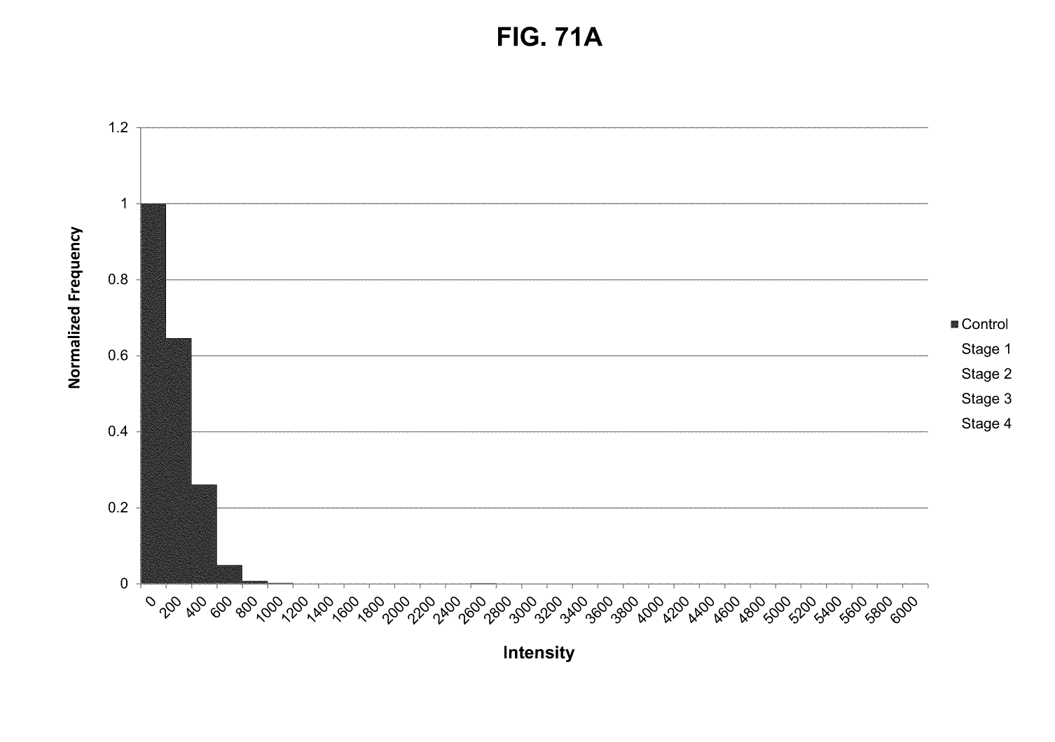

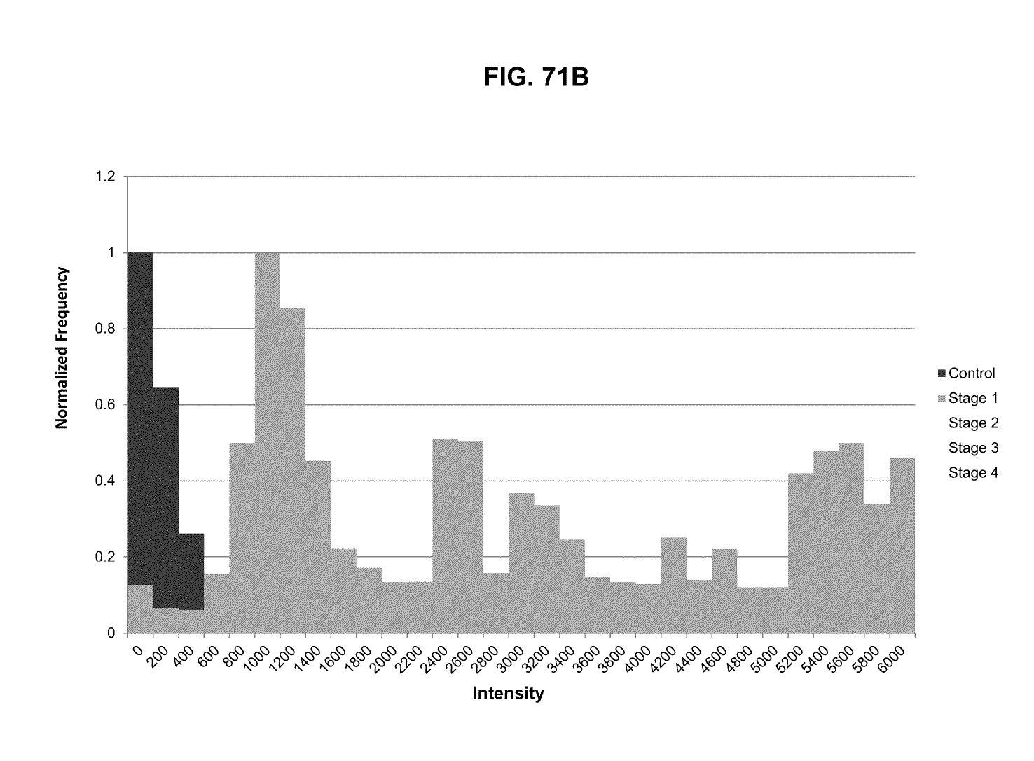

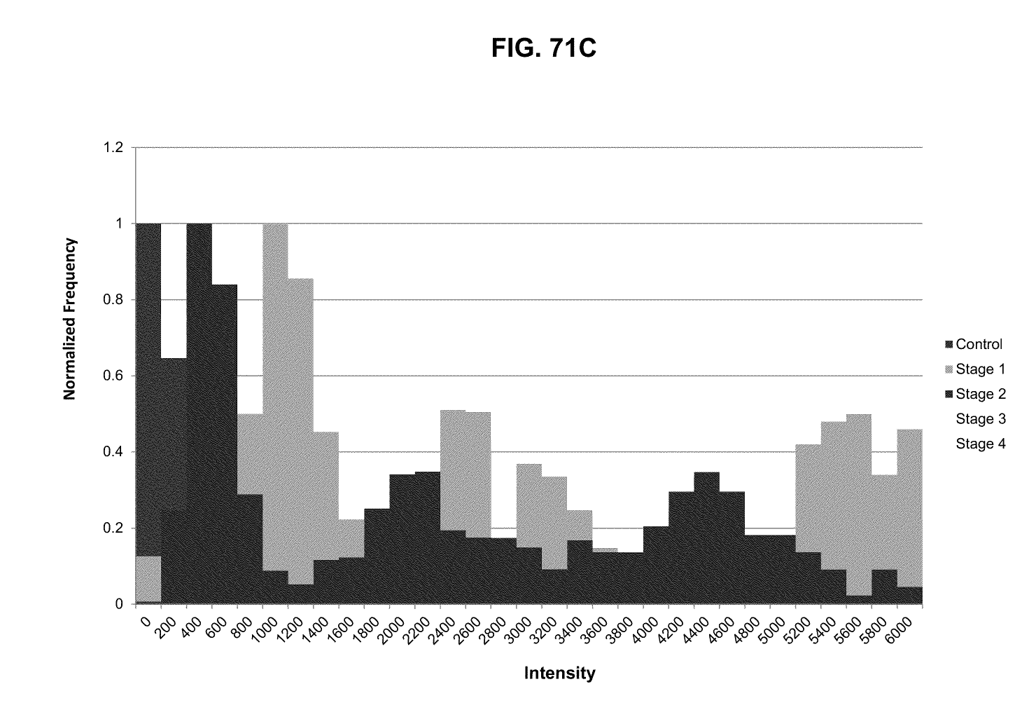

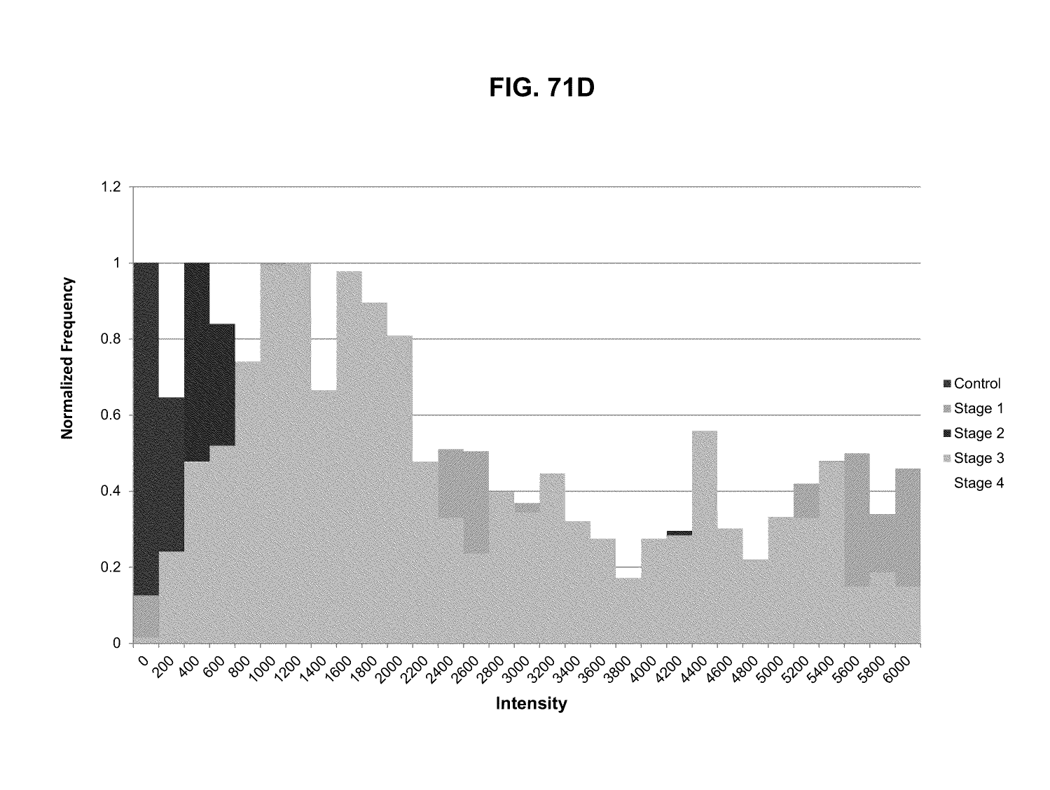

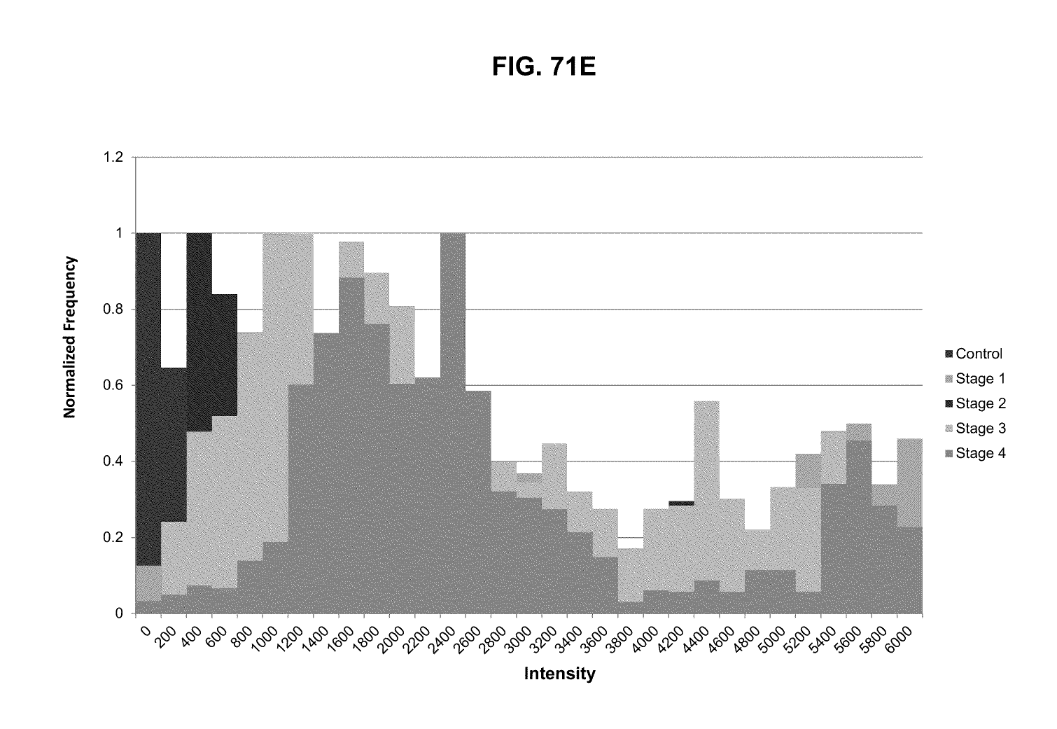

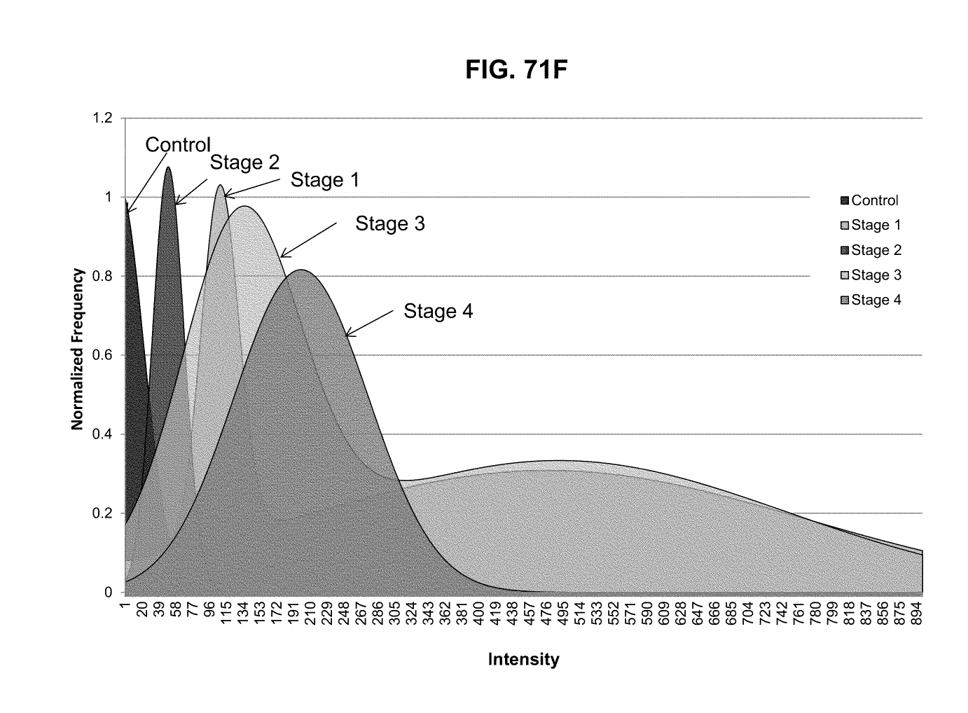

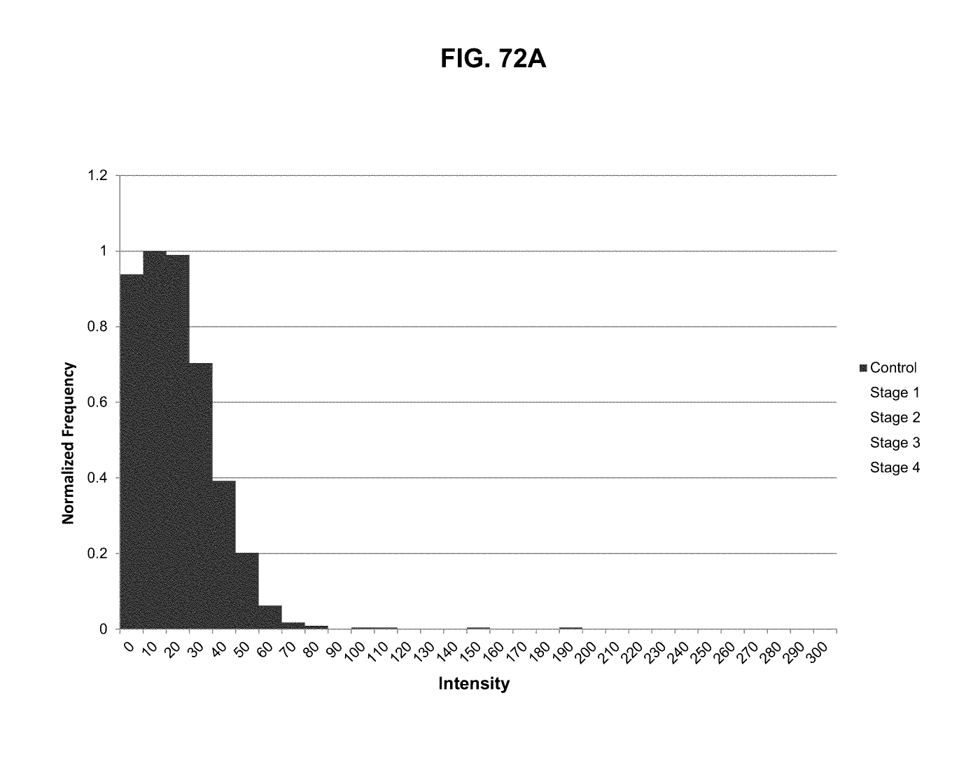

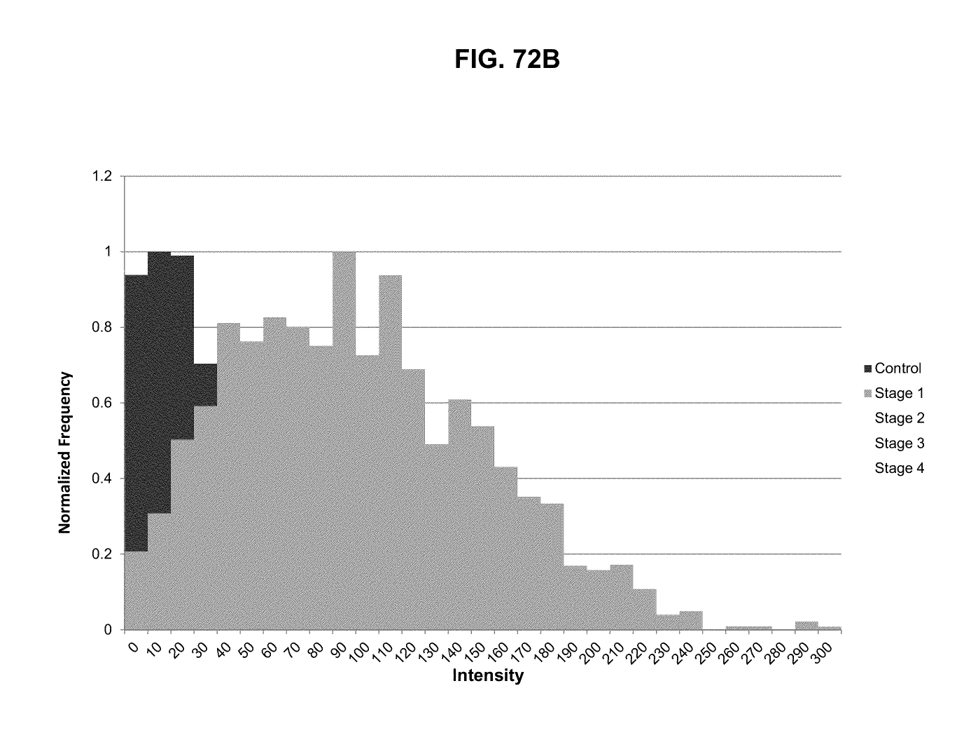

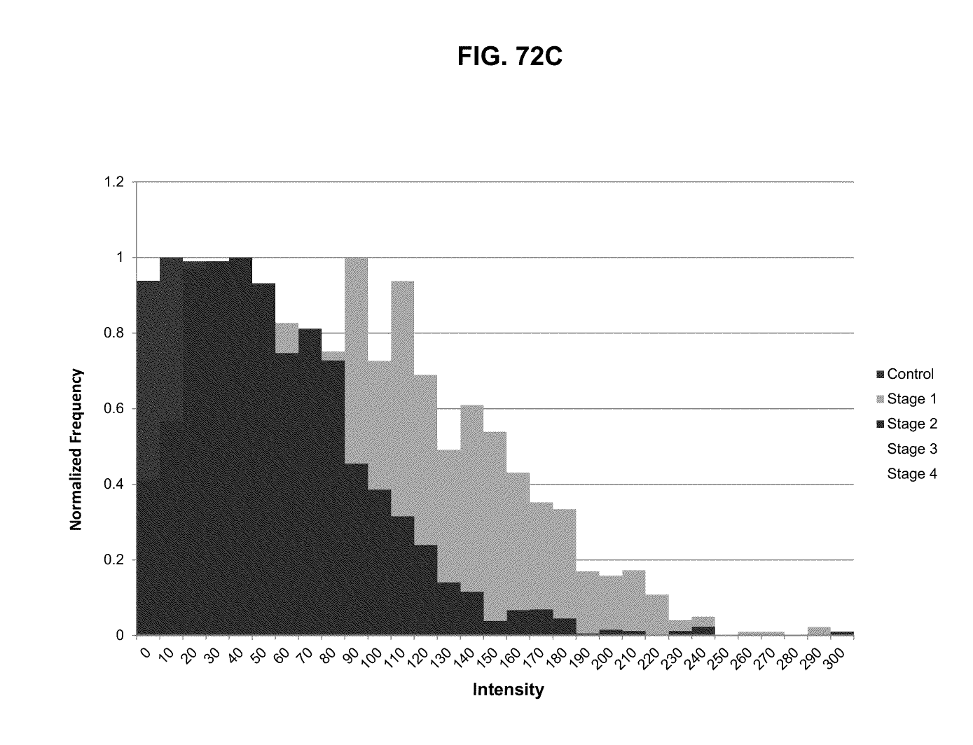

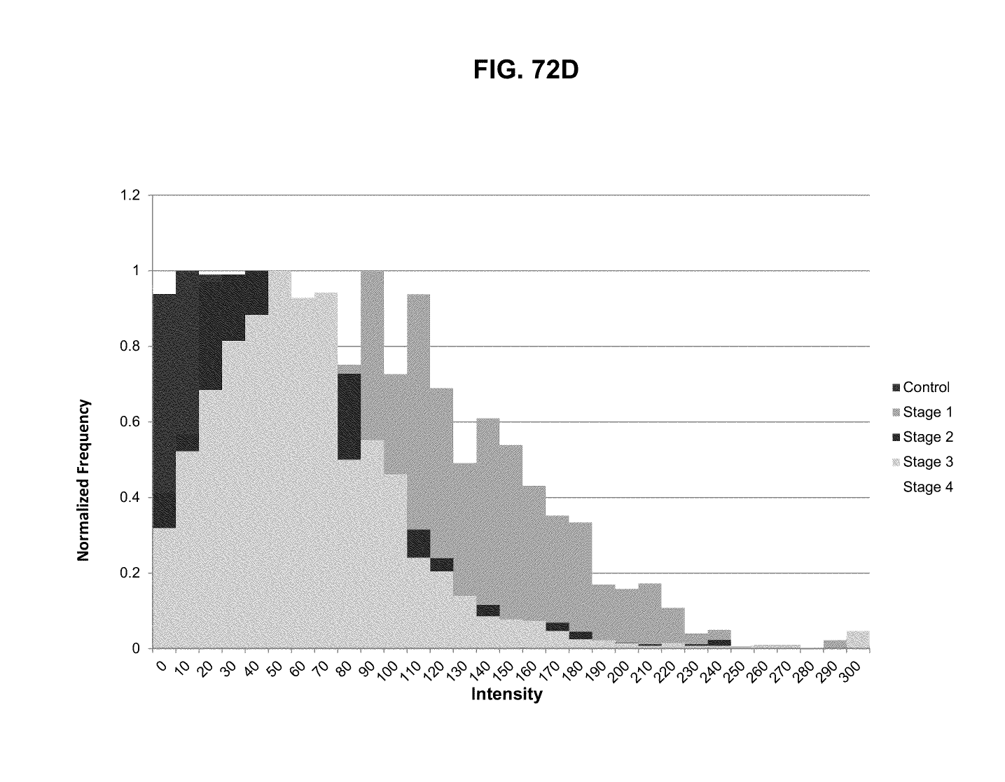

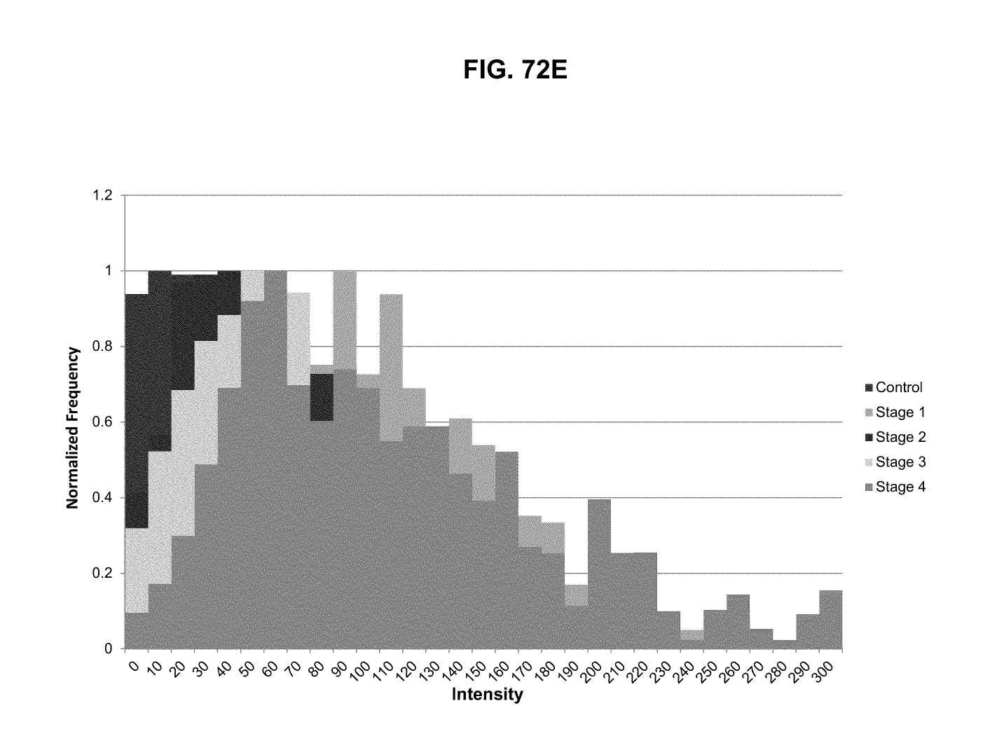

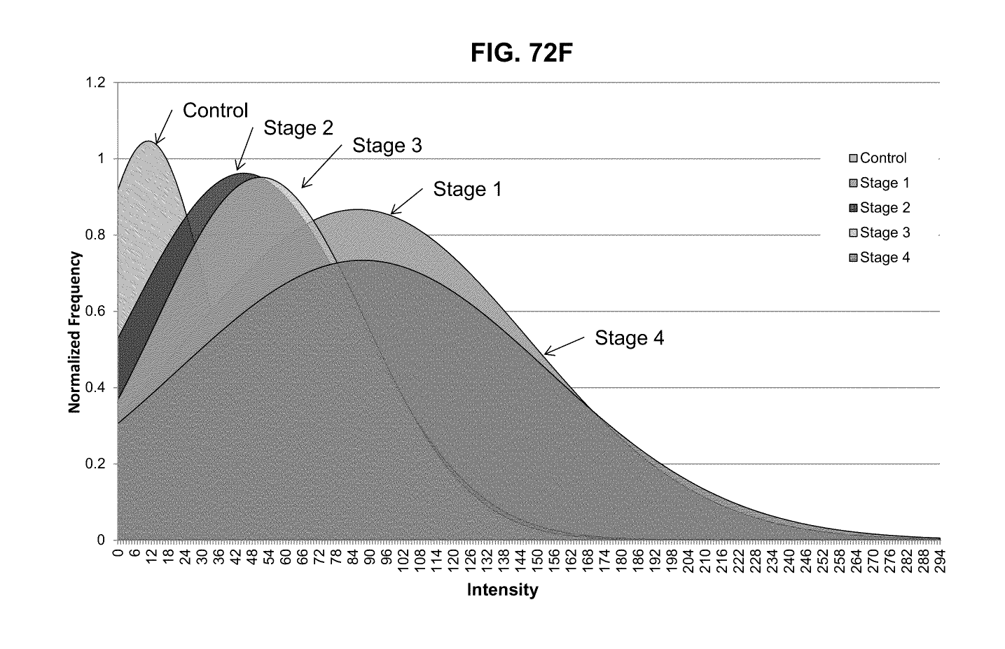

Moreover, an exosome bio-signature may be used to assess whether a subject is afflicted with disease, is at risk for developing disease or to assess the stage or progression of the disease. For example, a bio-signature can be used to assess whether a subject has prostate cancer (for example, FIGS. 68A-R, 73) or colon cancer (for example, FIGS. 69A-J, 74). Furthermore, a bio-signature can be used to determine a stage of a disease or condition, such as colon cancer (for example, FIGS. 71A-F, 72A-F).

Furthermore, determining the amount of exosomes, such a heterogeneous population of exosomes, and the amount of one or more homogeneous population of exosomes, such as a population of exosomes with the same bio-signature, can be used to characterize a phenotype. For example, determination of the total amount of exosomes in a sample (i.e. not cell-type specific) and determining the presence of one or more different cell-of-origin specific exosomes (such as cell-of-origin specific exosomes) can be used to characterize a phenotype. Threshold values, or reference values or amounts can be determined based on comparisons of normal subjects and subjects with the phenotype of interest, as further described below, and criteria based on the threshold or reference values determined. The different criteria can be used to characterize a phenotype.

For example, one criterion can be based on the amount of a heterogeneous population of exosomes in a sample. If the amount is lower than a threshold value or reference value, the criterion is met. Alternatively, the criterion can be based on whether the amount of exosomes is higher than a threshold or reference value. Another criterion can be the amount of exosomes with a specific bio-signature or biomarker. If the amount of exosomes with the specific bio-signature or biomarker is lower, or higher, than a threshold or reference value, the criterion is met. A criterion can also be based on the amount of exosomes derived from a particular cell type. If the amount is lower, or higher, than a threshold or reference value, the criterion is met. Another criterion can be based on whether the amount of exosomes derived from a cancer cell or comprising one or more cancer specific biomarkers. If the amount is lower, or higher, than a threshold or reference value, the criterion is met. A criterion can also be the reliability of the result, such as meeting a quality control measure or value.

A phenotype for a subject can be characterized based on meeting at least 1, 2, 3, 4, 5, 6, 7, 8, 9, or 10 criteria. For example, for the characterizing of a cancer, a number of different criteria can be used: 1) if the amount of exosomes in a sample from a subject is higher than a reference value; 2) if the amount of a cell type (ie. derived from a specific tissue or organ) specific exosomes is higher than a reference value; and 3) if the amount of exosomes with one or more cancer specific biomarkers is higher than a reference value, the subject is diagnosed with a cancer. The method can further include a quality control measure, such that the results are provided for the subject if the samples meet the quality control measure.

A bio-signature can be determined by comparing the amount of exosomes, the structure of an exosome, (using transmission electron microscopy, see for example, Hansen et al., Journal of Biomechanics 31, Supplement 1: 134-134(1) (1998), or scanning electron microscopy), or any other exosomal characteristic. Various combinations of methods and techniques or analyzing one or more exosomes can be used to determine a phenotype for a subject.

An exosome characteristic can include, but is not limited to the presence or absence, copy number, expression level, or activity level of a biomarker. The presence of a mutation (e.g., mutations which affect activity of the biomarker, such as substitution, deletion, or insertion mutations), variant, or post-translation modification of a biomarker, such as a protein biomarker, can include, but not be limited to, acylation, acetylation, phosphorylation, ubiquitination, deacetylation, alkylation, methylation, amidation, biotinylation, gamma-carboxylation, glutamylation, glycosylation, glycyation, hydroxylation, covalent attachment of heme moiety, iodination, isoprenylation, lipoylation, prenylation, GPI anchor formation, myristoylation, farnesylation, geranylgeranylation, covalent attachment of nucleotides or derivatives thereof, ADP-ribosylation, flavin attachment, oxidation, palmitoylation, pegylation, covalent attachment of phosphatidylinositol, phosphopantetheinylation, polysialylation, pyroglutamate formation, racemization of proline by prolyl isomerase, tRNA-mediation addition of amino acids such as arginylation, sulfation, the addition of a sulfate group to a tyrosine, or selenoylation of the biomarker can also be an exosomal characteristic.

The methods described above can be used to identify an exosome bio-signature that is associated with a disease, condition or physiological state.

The bio-signature can also be utilized to determine if a subject is afflicted with cancer or is at risk for developing cancer. A subject at risk of developing cancer can include those who may be predisposed or who have pre-symptomatic early stage disease.

A bio-signature can also be utilized to provide a diagnostic or theranostic determination for other diseases including but not limited to autoimmune diseases, inflammatory bowel diseases, Alzheimer's disease, Parkinson's disease, Multiple Sclerosis, sepsis or pancreatitis or any disease, conditions or symptoms listed in FIGS. 3-58.

The bio-signature can also be used to identify a given pregnancy state from the peripheral blood, umbilical cord blood, or amniotic fluid (e.g. miRNA signature specific to Downs Syndrome) or adverse pregnancy outcome such as pre-eclampsia, pre-term birth, premature rupture of membranes, intrauterine growth restriction or recurrent pregnancy loss. The bio-signature can also be used to indicate the health of the mother, the fetus at all developmental stages, the pre-implantation embryo or a newborn.

A bio-signature can be utilized for pre-symptomatic diagnosis. Furthermore, the bio-signature can be utilized to detect disease, determine disease stage or progression, determine the recurrence of disease, identify treatment protocols, determine efficacy of treatment protocols or evaluate the physiological status of individuals related to age and environmental exposure.

Monitoring the bio-signature of an exosome can also be used to identify toxic exposures in a subject including, but not limited to, situations of early exposure or exposure to an unknown or unidentified toxic agent. Without being bound by any one specific theory for mechanism of action, exosomes are shed from damaged cells and in the process compartmentalize specific contents of the cell including both membrane components and engulfed cytoplasmic contents. Cells exposed to toxic agents/chemicals may increase exosome shedding to expel toxic agents or metabolites thereof, thus resulting in increased exosome levels. Thus, monitoring an exosome and/or bio-signature allows assessment of an individual's response to potential toxic agent(s).

Furthermore, an exosome can be used to identify states of drug-induced toxicity or the organ injured, by detecting one or more specific antigen, binding agent, biomarker, or any combination thereof of the exosome. Therefore, the exosome, or exosome bio-signature can be used to monitor an individual for acute, chronic, or occupational exposures to any number of toxic agents including, but not limited to, drugs, antibiotics, industrial chemicals, toxic antibiotic metabolites, herbs, household chemicals, and chemicals produced by other organisms, either naturally occurring or synthetic in nature.

In addition, an exosome bio-signature can be used to identify conditions or diseases, including cancers of unknown origin, also known as cancers of unknown primary (CUP). For example, an exosome may be isolated from a biological sample as previously described to arrive at a heterogeneous population of exosomes. The heterogeneous population of exosomes can then be applied to surfaces coated with specific binding agents designed to rule out or identify antigen specific characteristics of the exosome population that are specific to a given cell-of-origin. Further, as described above, the bio-signature of a specific cell-of-origin exosome can correlate with the cancerous state of cells. Compounds that inhibit cancer in a subject may cause a change, e.g., a change in bio-signature of specific cell-of-origin exosome, which can be monitored by serial isolation of a cell-of-origin exosome over time and treatment course.

Alternatively, an exosome bio-signature can be used to assess the efficacy of a therapy, e.g., chemotherapy, radiation therapy, surgery, or any other therapeutic approach useful for inhibiting cancer in a subject. In addition, an exosome bio-signature can be used in a screening assay to identify candidate or test compounds or agents (e.g., proteins, peptides, peptidomimetics, peptoids, small molecules or other drugs) that have a modulatory effect on the bio-signature of a specific cell-of-origin exosome. Compounds identified via such screening assays may be useful, for example, for modulating, e.g., inhibiting, ameliorating, treating, or preventing conditions or diseases.

For example, a bio-signature for an exosome can be obtained from a patient who is undergoing successful treatment for a particular cancer. Cells from a cancer patient not being treated with the same drug can be cultured and exosomes from the cultures obtained for determining bio-signatures. The cells can be treated with test compounds and the bio-signature of the exosomes from the cultures can be compared to the bio-signature of the exosomes obtained from the patient undergoing successful treatment. The test compounds that results in exosome bio-signatures that are similar to those of the patient undergoing successful treatment can be selected for further studies.

The bio-signature of a specific cell-of-origin exosome can also be used to monitor the influence of an agent (e.g., drug compounds) on the bio-signature in clinical trials. Monitoring an exosome bio-signature can also be used in a method of assessing the efficacy of a test compound, such as a test compound for inhibiting cancer cells.

An exosome bio-signature can also be used to determine the effectiveness of a particular therapeutic intervention (pharmaceutical or non-pharmaceutical) and to alter the intervention to 1) reduce the risk of developing adverse outcomes, 2) enhance the effectiveness of the intervention or 3) identify resistant states. Thus, in addition to diagnosing or confirming the presence of or risk for developing a disease, condition or a syndrome, the methods and compositions disclosed herein also provide a system for optimizing the treatment of a subject having such a disease, condition or syndrome. For example, a therapy-related approach to treating a disease, condition or syndrome by integrating diagnostics and therapeutics to improve the real-time treatment of a subject can be determined by identifying the bio-signature of an exosome.

Tests that identify an exosome bio-signature can be used to identify which patients are most suited to a particular therapy, and provide feedback on how well a drug is working, so as to optimize treatment regimens. For example, in pregnancy-induced hypertension and associated conditions, therapy-related diagnostics can flexibly monitor changes in important parameters (e.g., cytokine and/or growth factor levels) over time, to optimize treatment.

Within the clinical trial setting of investigational agents as defined by the FDA, MDA, EMA, USDA, and EMEA, therapy-related diagnostics as determined by a bio-signature disclosed herein, can provide key information to optimize trial design, monitor efficacy, and enhance drug safety. For instance, for trial design, therapy-related diagnostics can be used for patient stratification, determination of patient eligibility (inclusion/exclusion), creation of homogeneous treatment groups, and selection of patient samples that are optimized to a matched case control cohort. Such therapy-related diagnostic can therefore provide the means for patient efficacy enrichment, thereby minimizing the number of individuals needed for trial recruitment. For example, for efficacy, therapy-related diagnostics are useful for monitoring therapy and assessing efficacy criteria. Alternatively, for safety, therapy-related diagnostics can be used to prevent adverse drug reactions or avoid medication error and monitor compliance with the therapeutic regimen.

Therefore, an exosomal bio-signature can be used to monitor drug efficacy, determine response or resistance to a given drug, and thereby enhance drug safety. For example, in colon cancer, exosomes are typically shed from colon cancer cells and can be isolated from the peripheral blood and used to isolate one or more biomarkers (e.g., KRAS mRNA). In the case of mRNA biomarkers, the mRNA can be reverse transcribed into cDNA and sequenced (e.g., by Sanger sequencing) to determine if there are mutations present that confer resistance to a drug (e.g., cetuximab or panitumimab).

In another example, exosomes that are specifically shed from lung cancer cells are isolated from a biological sample and used to isolate a lung cancer biomarker, e.g., EGFR mRNA. The EGFR mRNA is processed to cDNA and sequenced to determine if there are EGFR mutations present that show resistance or response to specific drugs or treatments for lung cancer.

One or more exosome bio-signatures can be grouped so that information obtained about the set of bio-signatures in a particular group provides a reasonable basis for making a clinically relevant decision, such as but not limited to a diagnosis, prognosis, or management of treatment, such as treatment selection.

As with most diagnostic markers, it is often desirable to use the fewest number of markers sufficient to make a correct medical judgment. This prevents a delay in treatment pending further analysis as well inappropriate use of time and resources.

Also disclosed herein are methods of conducting retrospective analysis on samples (e.g., serum and tissue biobanks) for the purpose of correlating qualitative and quantitative properties, such as exosome bio-signatures, with clinical outcomes in terms of disease state, disease stage, progression, prognosis; therapeutic efficacy or selection; or physiological conditions. Furthermore, methods and compositions disclosed herein are utilized for conducting prospective analysis on a sample (e.g., serum and/or tissue collected from individuals in a clinical trial) for the purpose of correlating qualitative and quantitative exosome bio-signatures with clinical outcomes in terms of disease state, disease stage, progression, prognosis; therapeutic efficacy or selection; or physiological conditions can also be performed. As used herein, exosome bio-signatures can be to cell-of-origin specific exosomes. Furthermore, bio-signatures can be determined based on an exosome surface marker profile and/or exosome contents (e.g., biomarkers).

An exosome bio-signature can comprise at least 2, 3, 4, 5, 6, 7, 8, 9, 10, 11, 12, 13, 14, 15, 16, 17, 18, 19, 20, 25, 30, 40, 50, 75, or 100 characteristics. A bio-signature with more than one exosomal characteristic, such as at least 2, 3, 4, 5, 6, 7, 8, 9, 10, 11, 12, 13, 14, 15, 16, 17, 18, 19, 20, 25, 30, 40, 50, 75, or 100 characteristics, may provide higher sensitivity, specificity, or both, in determining a phenotype. For example, assessing a plurality of exosomal characteristics can provide increased sensitivity, specificity, or both, as compared to assessing less than a plurality of exosomal characteristics.

A bio-signature comprising more than one exosomal characteristic can be used to characterize a phenotype with at least 60, 61, 62, 63, 64, 65, 66, 67, 68, 69, or 70% sensitivity, such as with at least 71, 72, 73, 74, 75, 76, 77, 78, 79, 80, 81, 82, 83, 84, 85, 86, or 87% sensitivity. For example, the phenotype can be characterized with at least 87.1, 87.2, 87.3, 87.4, 87.5, 87.6, 87.7, 87.8, 87.9, 88.0, or 89% sensitivity, such as at least 90% sensitivity. The phenotype can be characterized with at least 91, 92, 93, 94, 95, 96, 97, 98, 99 or 100% sensitivity.

The bio-signature can be used to characterize a phenotype of a subject with at least 70, 71, 72, 73, 74, 75, 76, 77, 78, 79, 80, 81, 82, 83, 84, 85, 86, 87, 88, 89, 90, 91, 92, 93, 94, 95, 96, or 97% specificity, such as with at least 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9 or 100% specificity.

The phenotype can also be characterized using a bio-signature with at least 70% sensitivity and at least 80, 90, 95, 99, or 100% specificity; at least 75% sensitivity and at least 80, 90, 95, 99, or 100% specificity; at least 80% sensitivity and at least 80, 85, 90, 95, 99, or 100% specificity; at least 85% sensitivity and at least 80, 85, 90, 95, 99, or 100% specificity; at least 86% sensitivity and at least 80, 85, 90, 95, 99, or 100% specificity; at least 87% sensitivity and at least 80, 85, 90, 95, 99, or 100% specificity; at least 88% sensitivity and at least 80, 85, 90, 95, 99, or 100% specificity; at least 89% sensitivity and at least 80, 85, 90, 95, 99, or 100% specificity; at least 90% sensitivity and at least 80, 85, 90, 95, 99, or 100% specificity; at least 95% sensitivity and at least 80, 85, 90, 95, 99, or 100% specificity; at least 99% sensitivity and at least 80, 85, 90, 95, 99, or 100% specificity; or at least 100% sensitivity and at least 80, 85, 90, 95, 99, or 100% specificity.

Furthermore, the confidence level for determining the specificity, sensitivity, or both, may be with at least 90, 91, 92, 93, 94, 95, 96, 97, 98, or 99% confidence.

### Bio-Signatures: Exosomal Biomarkers

An exosome bio-signature can comprise one or more biomarkers. An exosomal biomarker can be any component present in an exosome or on the exosome, such as any nucleic acid (e.g. RNA or DNA), protein, peptide, polypeptide, antigen, lipid, carbohydrate, or proteoglycan.

The bio-signature can include the presence or absence, expression level, mutational state, genetic variant state, or any modification (such as epigentic modification, post-translation modification) of a biomarker (e.g. any one or more biomarker listed in FIGS. 1, 3-60). The expression level of a biomarker can be compared to a control or reference, to determine the overexpression or underexpression (or upregulation or downregulation) of a biomarker in a sample. The control or reference level can be the amount of a biomarker, such as a miRNA in a control sample, such as a sample from a subject that does not have or exhibit the condition or disease, and further described below.

The nucleic acid can be any RNA or DNA species. For example, the biomarker can be mRNA, miRNA, small nucleolar RNAs (snoRNA), small nuclear RNAs (snRNA), ribosomal RNAs (rRNA), heterogeneous nuclear RNA (hnRNA), ribosomal RNAS (rRNA), siRNA, transfer RNAs (tRNA), or shRNA. The DNA can be double-stranded DNA, single stranded DNA, complementary DNA, or noncoding DNA.

In addition, the biomarker can be a polypeptide, peptides or protein, such as the modification state, truncations, mutations, expression level (such as overexpression or underexpression as compared to a reference level), and post-translational modifications, such as described above.

An exosome bio-signature may include a number of the same type of biomarkers (e.g., two different mRNAs, each corresponding to a different gene) or one or more of different types of biomarkers (e.g. mRNAs, miRNAs, proteins, peptides, ligands, and antigens).

One or more exosome bio-signatures can comprise at least one biomarker selected from those listed in FIGS. 1, 3-60. A specific cell-of-origin bio-signature may include one or more biomarkers. FIGS. 3-58 depict tables which lists a number of disease or condition specific biomarkers that can be derived and analyzed from an exosome. The biomarker can also beCD24, midkine, hepcidin, TMPRSS2-ERG, PCA-3, PSA, EGFR, EGFRvIII, BRAF variant, MET, cKit, PDGFR, Wnt, beta-catenin, K-ras, H-ras, N-ras, Raf, N-myc, c-myc, IGFR, PI3K, Akt, BRCA1, BRCA2, PTEN, VEGFR-2, VEGFR-1, Tie-2, TEM-1, CD276, HER-2, HER-3, or HER-4. The biomarker can also be annexin V, CD63, Rab-5b, or caveolin, or a miRNA, such as let-7a; miR-15b; miR-16; miR-19b; miR-21; miR-26a; miR-27a; miR-92; miR-93; miR-320 or miR-20. The biomarker can also be of any gene or fragment thereof as disclosed in PCT Publication No. WO2009/100029, such as those listed in Tables 3-15.

Other biomarkers useful for assessment in methods and compositions disclosed herein can include those associated with conditions or physiological states as disclosed in Rajendran et al., Proc Natl Acad Sci USA 2006; 103:11172-11177, Taylor et al., Gynecol Oncol 2008; 110:13-21, Zhou et al., Kidney Int 2008; 74:613-621, Buning et al., Immunology 2008, Prado et al. J Immunol 2008; 181:1519-1525, Vella et al. (2008) Vet Immunol Immunopathol 124(3-4): 385-93, Gould et al. (2003). Proc Natl Acad Sci USA 100(19): 10592-7, Fang et al. (2007). PLoS Biol 5(6): e158, Chen, B. J. and R. A. Lamb (2008). Virology 372(2): 221-32, Bhatnagar, S. and J. S. Schorey (2007). J Biol Chem 282(35): 25779-89, Bhatnagar et al. (2007) Blood 110(9): 3234-44, Yuyama, et al. (2008). J Neurochem 105(1): 217-24, Gomes et al. (2007). Neurosci Lett 428(1): 43-6, Nagahama et al. (2003). Autoimmunity 36(3): 125-31, Taylor, D. D., S. Akyol, et al. (2006). J Immunol 176(3): 1534-42, Peche, et al. (2006). Am J Transplant 6(7): 1541-50, Zero, M., M. Valenti, et al. (2008). Cell Death and Differentiation 15: 80-88, Gesierich, S., I. Berezoversuskiy, et al. (2006), Cancer Res 66(14): 7083-94, Clayton, A., A. Turkes, et al. (2004). Faseb J 18(9): 977-9, Skriner., K Adolph, et al. (2006). Arthritis Rheum 54(12): 3809-14, Brouwer, R., G. J. Pruijn, et al. (2001). Arthritis Res 3(2): 102-6, Kim, S. H., N. Bianco, et al. (2006). Mol Ther 13(2): 289-300, Evans, C. H., S. C. Ghivizzani, et al. (2000). Clin Orthop Relat Res (379 Suppl): S300-7, Zhang, H. G., C. Liu, et al. (2006). J Immunol 176(12): 7385-93, Van Niel, G., J. Mallegol, et al. (2004). Gut 52: 1690-1697, Fiasse, R. and O. Dewit (2007). Expert Opinion on Therapeutic Patents 17(12): 1423-1441(19).

A biomarker that can be derived and analyzed from an exosome includes, but is not limited to, the presence or absence, expression level, mutations (for example genetic mutations, such as deletions, translocations, duplications, nucleotide or amino acid substitutions, and the like) of miRNA (miR) and miRNA*nonsense (miR*), and other RNAs (including, but not limited to, mRNA, preRNA, priRNA, hnRNA, snRNA, siRNA, shRNA), DNA, proteins, peptides, and ligands. Any epigenetic modulation or copy number variation of a biomarker can also be analyzed. A miRNA biomarker includes not only its miRNA and microRNA* nonsense, but its precursor molecules: pri-microRNAs (pri-miRs) and pre-microRNAs (pre-miRs) are also included as biomarkers. The sequence of a miRNA can be obtained from publicly available databases such as www.mirbase.org, www.microrna.org, or any others available.

The one or more biomarkers analyzed from an exosome can be indicative of a particular tissue or cell of origin, disease, or physiological state, as further described below. Furthermore, the presence, absence or expression level of one or more of the biomarkers described herein can be correlated to a phenotype of a subject, including a disease, condition, prognosis or drug efficacy. The specific biomarker and bio-signature set forth below constitute non-inclusive examples for each of the diseases, condition comparisons, conditions, and/or physiological states. Furthermore, the one or more biomarker assessed for a phenotype can be a cell-of-origin specific exosome, such as those described above.

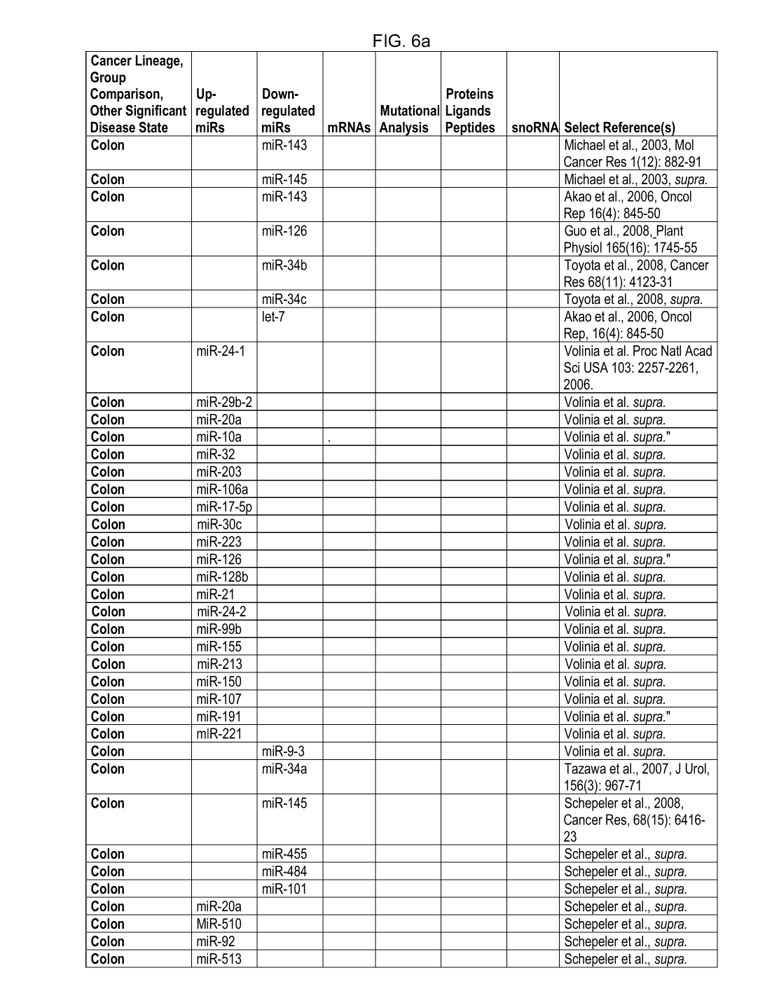

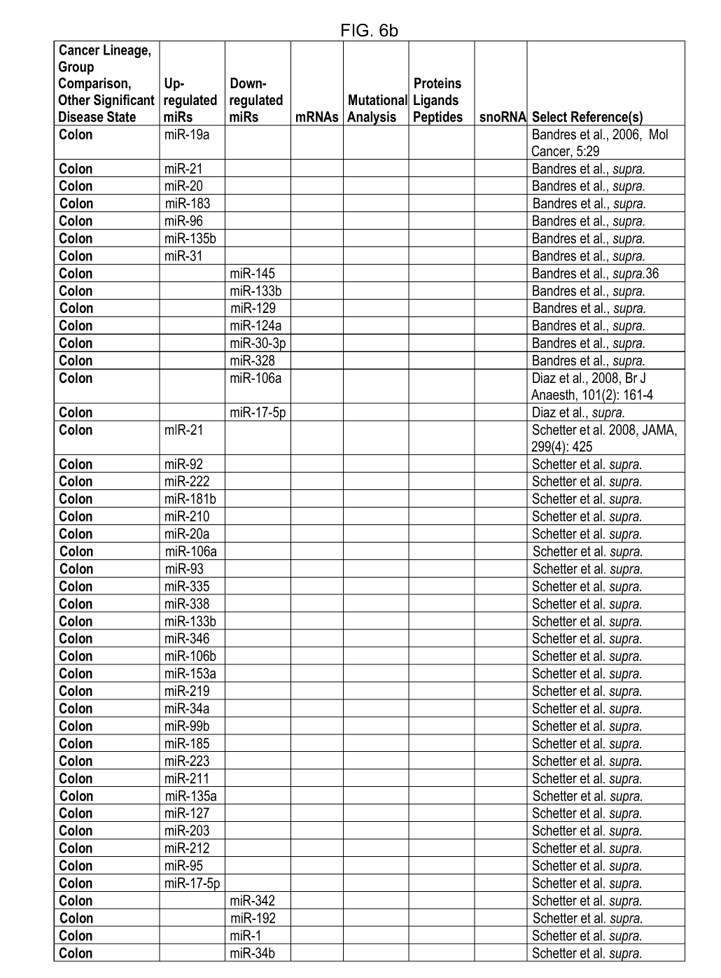

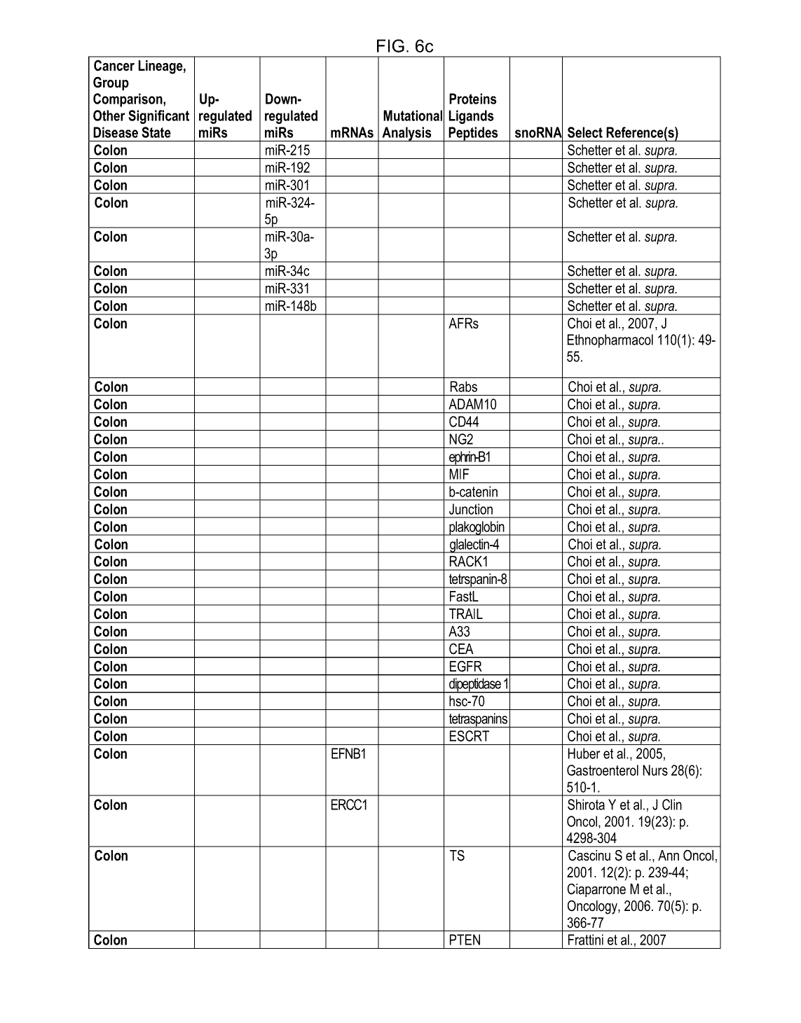

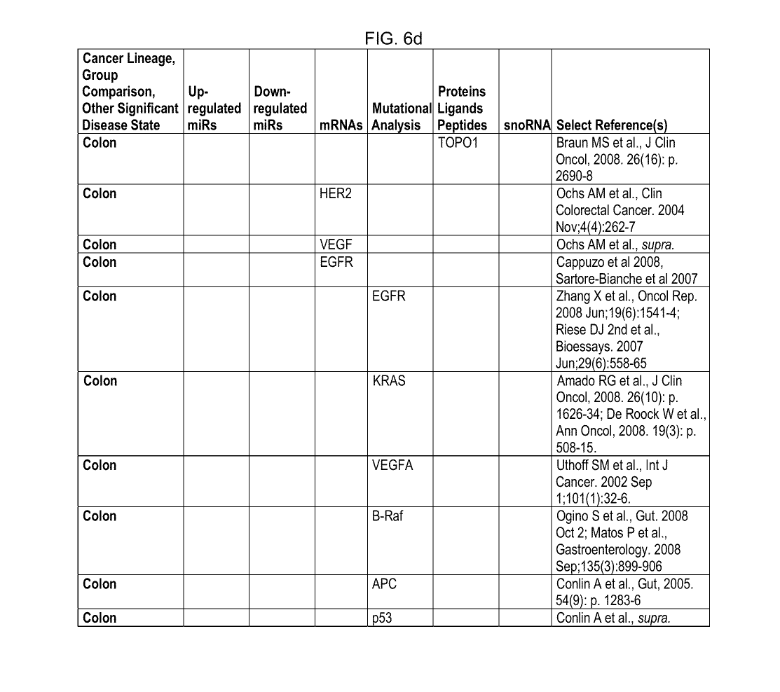

The one or more miRNAs used to characterize a phenotype may be selected from those disclosed in PCT Publication No. WO2009/036236. For example, one or more miRNAs listed in Tables I-VI (FIGS. 6-11) can be used to characterize colon adenocarcinoma, colorectal cancer, prostate cancer, lung cancer, breast cancer, b-cell lymphoma, pancreatic cancer, diffuse large BCL cancer, CLL, bladder cancer, renal cancer, hypoxia-tumor, uterine leiomyomas, ovarian cancer, hepatitis C virus-associated hepatocellular carcinoma, ALL, Alzheimer's disease, myelofibrosis, myelofibrosis, polycythemia vera, thrombocythemia, HIV, or HIV-I latency, as further described herein.

The one or more miRNAs can be detected in plasma exosomes. The one or more miRNAs can be miR-223, miR-484, miR-191, miR-146a, miR-016, miR-026a, miR-222, miR-024, miR-126, and miR-32. One or more miRNAs can also be detected in PBMC. The one or more miRNAs can be miR-223, miR-150, miR-146b, miR-016, miR-484, miR-146a, miR-191, miR-026a, miR-019b, or miR-020a. The one or more miRNAs can be used to characterize a particular disease or condition. For example, for the disease bladder cancer, one or more miRNAs can be detected, such as miR-223, miR-26b, miR-221, miR-103-1, miR-185, miR-23b, miR-203, miR-17-5p, miR-23a, miR-205 or any combination thereof. The one or more miRNAs may be upregulated or overexpressed.

In some embodiments, the one or more miRNAs is used to characterize hypoxia-tumor. The one or more miRNA may be miR-23, miR-24, miR-26, miR-27, miR-103, miR-107, miR-181, miR-210, or miR-213, and may be upregulated. One or more miRNAs can also be used to characterize uterine leiomyomas. For example, the one or more miRNAs used to characterize a uterine leiomyoma may be a let-7 family member, miR-21, miR-23b, miR-29b, or miR-197. The miRNA can be upregulated.

Myelofibrosis can also be characterized by one or more miRNAs, such as miR-190, which can be upregulated; miR-31, miR-150 and miR-95, which can be downregulated, or any combination thereof. Furthermore, myelofibrosis, polycythemia vera or thrombocythemia can also be characterized by detecting one or more miRNAs, such as, but not limited to, miR-34a, miR-342, miR-326, miR-105, miR-149, miR-147, or any combination thereof. The one or more miRNAs may be downregulated.

Other examples of phenotypes that can be characterized by assessing an exosome for one or more biomarkers are further described herein.

The one or more biomarkers can be detected by a probe. A probe can comprise of an oligonucelotide, such as DNA or RNA, an aptamer, monoclonal antibody, polyclonal antibody, Fabs, Fab′, single chain antibody, synthetic antibody, peptoid, zDNA, peptide nucleic acid (PNA), locked nucleic acid (LNA), lectin, synthetic or naturally occurring chemical compound (including but not limited to a drug or labeling reagent), dendrimer, or any combination thereof. The probe can be directly detected, for example by being directly labeled, or be indirectly detected, such as through a labeling reagent. The probe can selectively hybridize to a biomarker. For example, a probe that is an oligonucleotide can selectively hybridize to a miRNA biomarker.

Breast Cancer

Breast cancer specific biomarkers can include one or more (for example, 2, 3, 4, 5, 6, 7, 8, or more) overexpressed miRs, underexpressed miRs, mRNA, genetic mutations, proteins, ligands, peptides, snoRNA, or any combination thereof, such as listed in FIG. 3.

One or more breast cancer specific biomarker can be assessed to provide a breast cancer specific exosome bio-signature. For example, the bio-signature can comprise one or more overexpressed miRs, including but not limited to, miR-21, miR-155, miR-206, miR-122a, miR-210, miR-21, miR-21, miR-155, miR-206, miR-122a, miR-210, or miR-21, or any combination thereof.

The bio-signature can also comprise one or more underexpressed miRs such as, but not limited to, let-7, miR-10b, miR-125a, miR-125b, miR-145, miR-143, miR-145, miR-16, let-7, let-7, let-7, miR-10b, miR-125a, miR-125b, or miR-145, or any combination thereof.

The mRNAs that may be analyzed can include, but are not limited to, ER, PR, HER2, MUC1, or EGFR, or any combination thereof. Mutations including, but not limited to, those related to KRAS, B-Raf, or CYP2D6, or any combination thereof can also be used as specific biomarkers from exosomes for breast cancer. In addition, a protein, ligand, or peptide that can be used as biomarkers from exosomes that are specific to breast cancer includes, but are not limited to, hsp70, MART-1, TRP, HER2, hsp70, MART-1, TRP, HER2, ER, PR, Class III b-tubulin, or VEGFA, or any combination thereof. Furthermore the snoRNA that can be used as an exosomal biomarker for breast cancer include, but are not limited to, GAS5. The gene fusion ETV6-NTRK3 can also be used a biomarker for breast cancer.

Also provided herein is an isolated exosome comprising one or more breast cancer specific biomarkers, such as ETV6-NTRK3, or biomarkers listed in FIG. 3 and in FIG. 1 for breast cancer. A composition comprising the isolated exosome is also provided. Accordingly, in some embodiments, the composition comprises a population of exosomes comprising one or more breast cancer specific biomarkers, such as ETV6-NTRK3, or biomarkers listed in FIG. 3 and in FIG. 1 for breast cancer. The composition can comprise a substantially enriched population of exosomes, wherein the population of exosomes is substantially homogeneous for breast cancer specific exosomes or exosomes comprising one or more breast cancer specific biomarkers, such as ETV6-NTRK3, or biomarkers listed in FIG. 3 and in FIG. 1 for breast cancer.

One or more breast cancer specific biomarkers, such as ETV6-NTRK3, or biomarkers listed in FIG. 3 and in FIG. 1 for breast cancer can also be detected by one or more systems disclosed herein, for characterizing a breast cancer. For example, a detection system can comprise one or more probes to detect one or more breast cancer specific biomarkers, such as ETV6-NTRK3, or biomarkers listed in FIG. 3 and in FIG. 1 for breast cancer, of one or more exosomes of a biological sample.

Ovarian Cancer

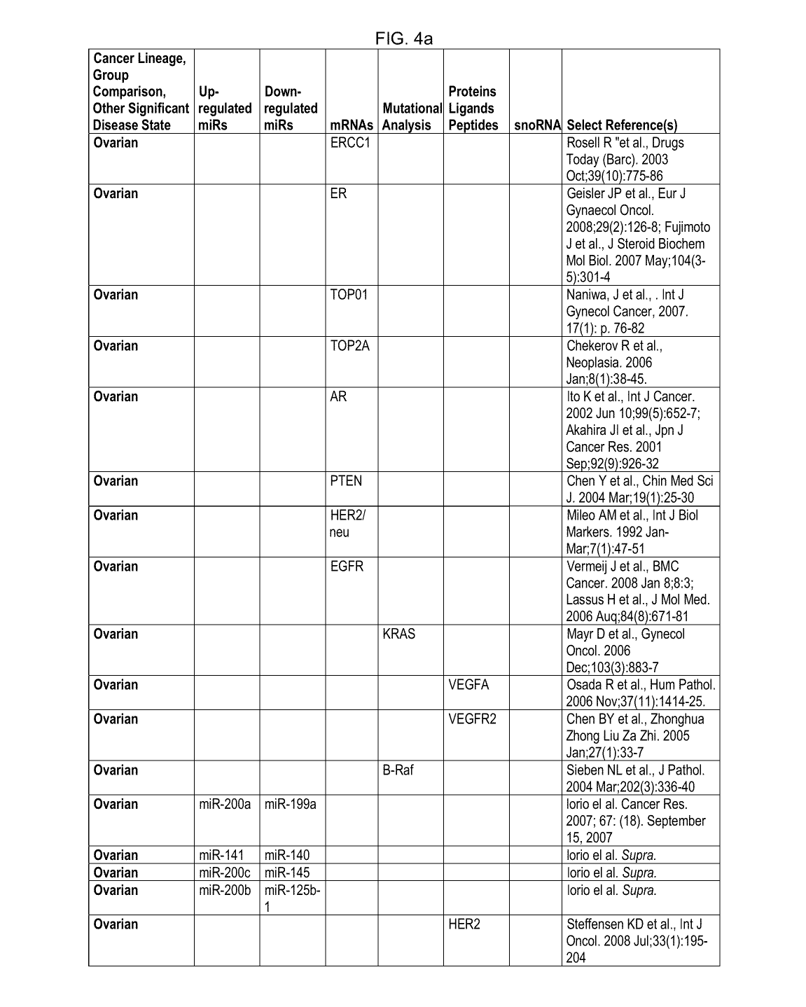

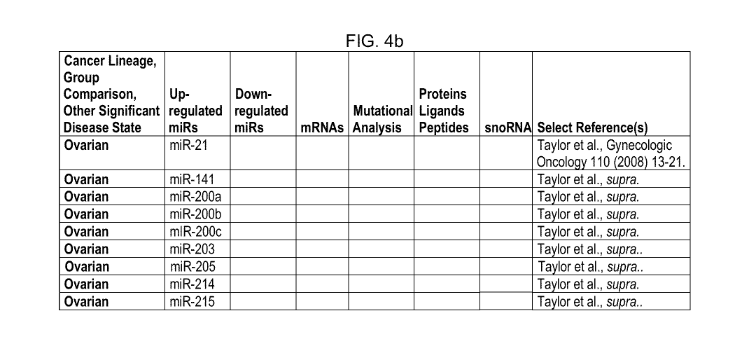

Ovarian cancer specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 4, and can be used to create a ovarian cancer specific exosome bio-signature. For example, the bio-signature can comprise one or more overexpressed miRs, such as, but not limited to, miR-200a, miR-141, miR-200c, miR-200b, miR-21, miR-141, miR-200a, miR-200b, miR-200c, miR-203, miR-205, miR-214, miR-199*, or miR-215, or any combination thereof. The bio-signature can also comprise one or more underexpressed miRs such as, but not limited to, miR-199a, miR-140, miR-145, miR-100, miR-let-7 cluster, or miR-125b-1, or any combination thereof. The one or more mRNAs that may be analyzed can include, but are not limited to, ERCC1, ER, TOPO1, TOP2A, AR, PTEN, HER2/neu, CD24 or EGFR, or any combination thereof.

A biomarker mutation for ovarian cancer that can be assessed in an exosome includes, but is not limited to, a mutation of KRAS, mutation of B-Raf, or any combination of mutations specific for ovarian cancer. The protein, ligand, or peptide that can be assessed in an exosome can include, but is not limited to, VEGFA, VEGFR2, or HER2, or any combination thereof. Furthermore, an exosome isolated or assayed can be ovarian cancer cell specific, or derived from ovarian cancer cells.

Also provided herein is an isolated exosome comprising one or more ovarian cancer specific biomarkers, such as CD24, those listed in FIG. 4 and in FIG. 1 for ovarian cancer. A composition comprising the isolated exosome is also provided. Accordingly, in some embodiments, the composition comprises a population of exosomes comprising one or more ovarian cancer specific biomarkers, such as CD24, those listed in FIG. 4 and in FIG. 1 for ovarian cancer. The composition can comprise a substantially enriched population of exosomes, wherein the population of exosomes is substantially homogeneous for ovarian cancer specific exosomes or exosomes comprising one or more ovarian cancer specific biomarkers, such as CD24, those listed in FIG. 4 and in FIG. 1 for ovarian cancer.

One or more ovarian cancer specific biomarkers, such as CD24, those listed in FIG. 4 and in FIG. 1 for ovarian cancer can also be detected by one or more systems disclosed herein, for characterizing an ovarian cancer. For example, a detection system can comprise one or more probes to detect one or more ovarian cancer specific biomarkers, such as CD24, those listed in FIG. 4 and in FIG. 1 for ovarian cancer, of one or more exosomes of a biological sample.

Lung Cancer

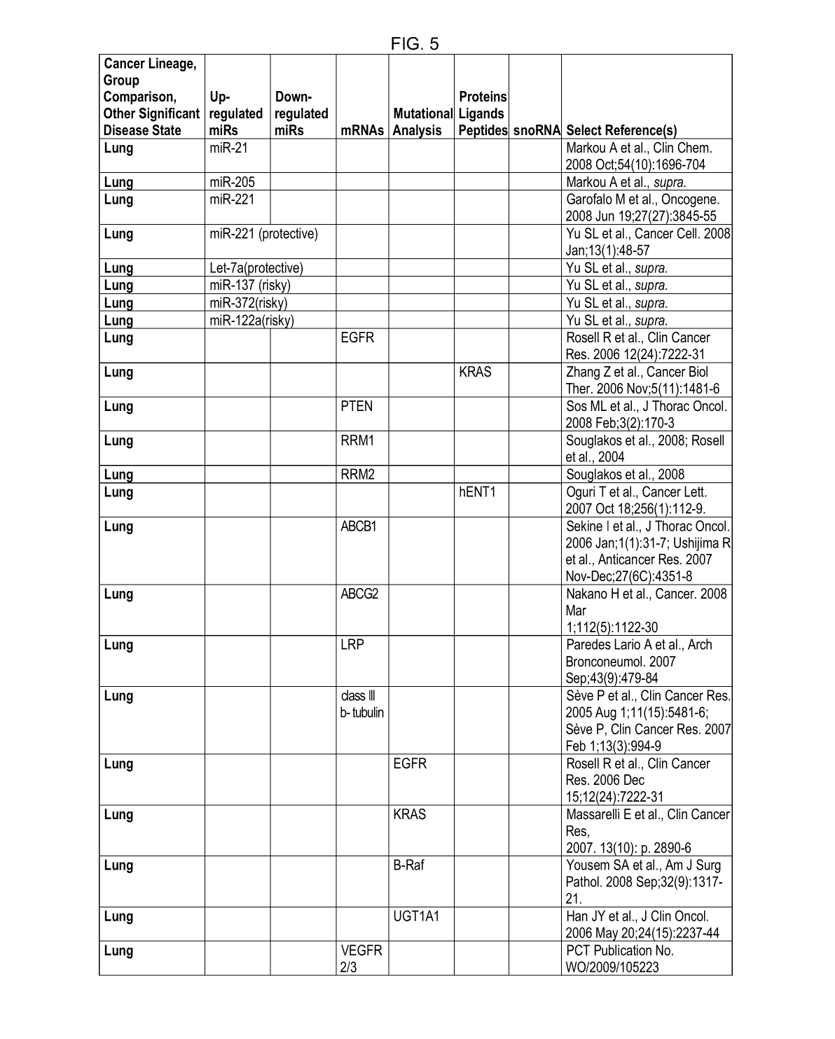

Lung cancer specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 5, and can be used to create a lung cancer specific exosome bio-signature.

The bio-signature can comprise one or more overexpressed miRs, such as, but not limited to, miR-21, miR-205, miR-221 (protective), let-7a (protective), miR-137 (risky), miR-372 (risky), or miR-122a (risky), or any combination thereof. The bio-signature can comprise one or more upregulated or overexpressed miRNAs, such as miR-17-92, miR-19a, miR-21, miR-92, miR-155, miR-191, miR-205 or miR-210; one or more downregulated or underexpressed miRNAs, such as miR-let-7, or any combination thereof.

The one or more mRNAs that may be analyzed can include, but are not limited to, EGFR, PTEN, RRM1, RRM2, ABCB1, ABCG2, LRP, VEGFR2, VEGFR3, class III b-tubulin, or any combination thereof.

A biomarker mutation for lung cancer that can be assessed in an exosome includes, but is not limited to, a mutation of EGFR, KRAS, B-Raf, UGT1A1, or any combination of mutations specific for lung cancer. The protein, ligand, or peptide that can be assessed in an exosome can include, but is not limited to, KRAS, hENT1, or any combination thereof.

The biomarker can also be midkine (MK or MDK). Furthermore, an exosome isolated or assayed can be lung cancer cell specific, or derived from lung cancer cells.

Also provided herein is an isolated exosome comprising one or more lung cancer specific biomarkers, such as RLF-MYCL1, TGF-ALK, or CD74-ROS1, or those listed in FIG. 5 and in FIG. 1 for lung cancer. A composition comprising the isolated exosome is also provided. Accordingly, in some embodiments, the composition comprises a population of exosomes comprising one or more lung cancer specific biomarkers, such as RLF-MYCL1, TGF-ALK, or CD74-ROS1, or those listed in FIG. 5 and in FIG. 1 for lung cancer. The composition can comprise a substantially enriched population of exosomes, wherein the population of exosomes is substantially homogeneous for lung cancer specific exosomes or exosomes comprising one or more lung cancer specific biomarkers, such as RLF-MYCL1, TGF-ALK, or CD74-ROS1, or those listed in FIG. 5 and in FIG. 1 for lung cancer.

One or more lung cancer specific biomarkers, such as RLF-MYCL1, TGF-ALK, or CD74-ROS1, or those listed in FIG. 5 and in FIG. 1 for lung cancer can also be detected by one or more systems disclosed herein, for characterizing a lung cancer. For example, a detection system can comprise one or more probes to detect one or more lung cancer specific biomarkers, such as RLF-MYCL1, TGF-ALK, or CD74-ROS1, or those listed in FIG. 5 and in FIG. 1 for lung cancer, of one or more exosomes of a biological sample.

Colon Cancer

Colon cancer specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 6, and can be used to create a colon cancer specific exosome bio-signature. For example, the bio-signature can comprise one or more overexpressed miRs, such as, but not limited to, miR-24-1, miR-29b-2, miR-20a, miR-10a, miR-32, miR-203, miR-106a, miR-17-5p, miR-30c, miR-223, miR-126, miR-128b, miR-21, miR-24-2, miR-99b, miR-155, miR-213, miR-150, miR-107, miR-191, miR-221, miR-20a, miR-510, miR-92, miR-513, miR-19a, miR-21, miR-20, miR-183, miR-96, miR-135b, miR-31, miR-21, miR-92, miR-222, miR-181b, miR-210, miR-20a, miR-106a, miR-93, miR-335, miR-338, miR-133b, miR-346, miR-106b, miR-153a, miR-219, miR-34a, miR-99b, miR-185, miR-223, miR-211, miR-135a, miR-127, miR-203, miR-212, miR-95, or miR-17-5p, or any combination thereof. The bio-signature can also comprise one or more underexpressed miRs such as miR-143, miR-145, miR-143, miR-126, miR-34b, miR-34c, let-7, miR-9-3, miR-34a, miR-145, miR-455, miR-484, miR-101, miR-145, miR-133b, miR-129, miR-124a, miR-30-3p, miR-328, miR-106a, miR-17-5p, miR-342, miR-192, miR-1, miR-34b, miR-215, miR-192, miR-301, miR-324-5p, miR-30a-3p, miR-34c, miR-331, or miR-148b, or any combination thereof.

The one or more biomarker can be an upregulated or overexpressed miRNA, such as miR-20a, miR-21, miR-106a, miR-181b or miR-203, for characterizing a colon adenocarcinoma. The one or more biomarker can be used to characterize a colorectal cancer, such as an upregulated or overexpressed miRNA selected from the group consisting of: miR-19a, miR-21, miR-127, miR-31, miR-96, miR-135b and miR-183, a downregulated or underexpressed miRNA, such as miR-30c, miR-133a, mir143, miR-133b or miR-145, or any combination thereof.

The one or more mRNAs that may be analyzed can include, but are not limited to, EFNB1, ERCC1, HER2, VEGF, or EGFR, or any combination thereof. A biomarker mutation for colon cancer that can be assessed in an exosome includes, but is not limited to, a mutation of EGFR, KRAS, VEGFA, B-Raf, APC, or p53, or any combination of mutations specific for colon cancer. The protein, ligand, or peptide that can be assessed in an exosome can include, but is not limited to, AFRs, Rabs, ADAM10, CD44, NG2, ephrin-B1, MIF, b-catenin, Junction, plakoglobin, glalectin-4, RACK1, tetrspanin-8, FasL, TRAIL, A33, CEA, EGFR, dipeptidase 1, hsc-70, tetraspanins, ESCRT, TS, PTEN, or TOPO1, or any combination thereof. Furthermore, an exosome isolated or assayed can be colon cancer cell specific, or derived from colon cancer cells.

Also provided herein is an isolated exosome comprising one or more colon cancer specific biomarkers, such as listed in FIG. 6 and in FIG. 1 for colon cancer. A composition comprising the isolated exosome is also provided. Accordingly, in some embodiments, the composition comprises a population of exosomes comprising one or more colon cancer specific biomarkers, such as listed in FIG. 6 and in FIG. 1 for colon cancer. The composition can comprise a substantially enriched population of exosomes, wherein the population of exosomes is substantially homogeneous for colon cancer specific exosomes or exosomes comprising one or more colon cancer specific biomarkers, such as listed in FIG. 6 and in FIG. 1 for colon cancer.

One or more colon cancer specific biomarkers, such as listed in FIG. 6 and in FIG. 1 for colon cancer can also be detected by one or more systems disclosed herein, for characterizing a colon cancer. For example, a detection system can comprise one or more probes to detect one or more colon cancer specific biomarkers, such as listed in FIG. 6 and in FIG. 1 for colon cancer, of one or more exosomes of a biological sample.

Adenoma Versus Hyperplastic Polyp

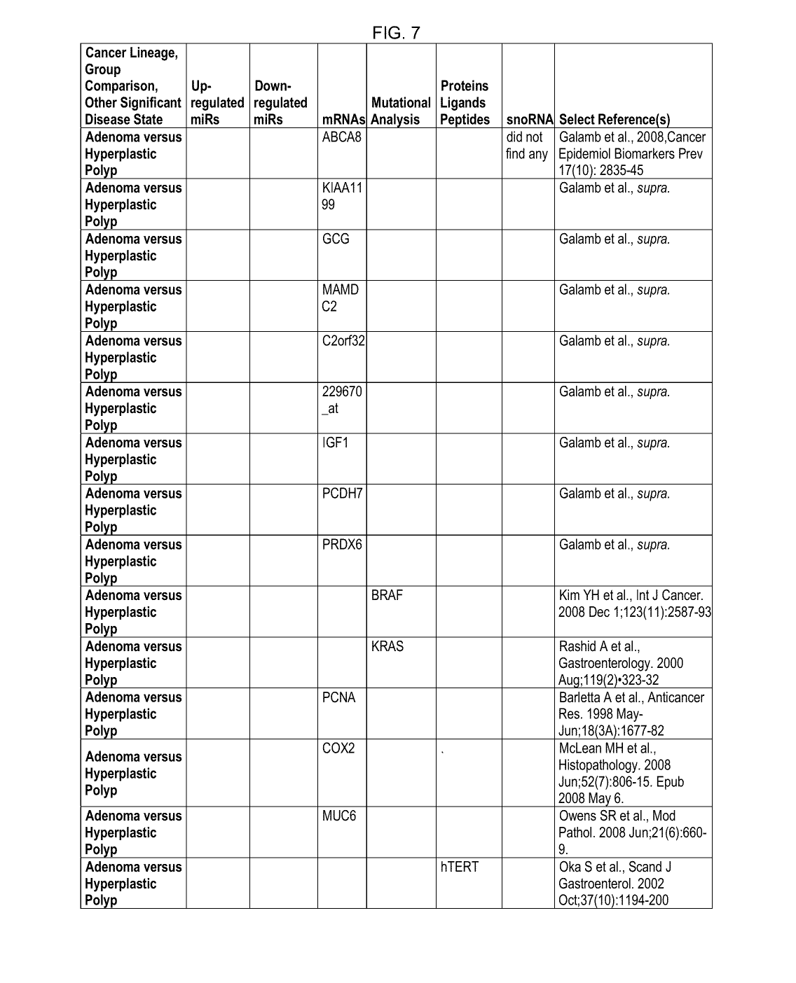

Adenoma versus hyperplastic polyp specific biomarkers from exosomes can include one or more (for example, 2, 3, 4, 5, 6, 7, 8, or more) overexpressed miRs, underexpressed miRs, mRNAs, genetic mutations, proteins, ligands, peptides, or any combination thereof, such as listed in FIG. 7, and can be used to create an adenoma versus hyperplastic polyp specific exosome bio-signature. For example, the one or more mRNAs that may be analyzed can include, but are not limited to, ABCA8, KIAA1199, GCG, MAMDC2, C2orf32, 229670_at, IGF1, PCDH7, PRDX6, PCNA, COX2, or MUC6, or any combination thereof.

A biomarker mutation to distinguish for adenoma versus hyperplastic polyp that can be assessed in an exosome includes, but is not limited to, a mutation of KRAS, mutation of B-Raf, or any combination of mutations specific for distinguishing between adenoma versus hyperplastic polyp. The protein, ligand, or peptide that can be assessed in an exosome can include, but is not limited to, hTERT.

Also provided herein is an isolated exosome comprising one or more specific biomarkers for distinguishing between an adenoma and a hyperplastic polyp, such as listed in FIG. 7. A composition comprising the isolated exosome is also provided. Accordingly, in some embodiments, the composition comprises a population of exosomes comprising one or more specific biomarkers for distinguishing between an adenoma and a hyperplastic polyp, such as listed in FIG. 7. The composition can comprise a substantially enriched population of exosomes, wherein the population of exosomes is substantially homogeneous for having one or more specific biomarkers for distinguishing between an adenoma and a hyperplastic polyp, such as listed in FIG. 7.

One or more specific biomarkers for distinguishing between an adenoma and a hyperplastic polyp, such as listed in FIG. 7 can also be detected by one or more systems disclosed herein, for distinguishing between an adenoma and a hyperplastic polyp. For example, a detection system can comprise one or more probes to detect one or more specific biomarkers for distinguishing between an adenoma and a hyperplastic polyp, such as listed in FIG. 7, of one or more exosomes of a biological sample.

Irritable Bowel Disease (IBD)

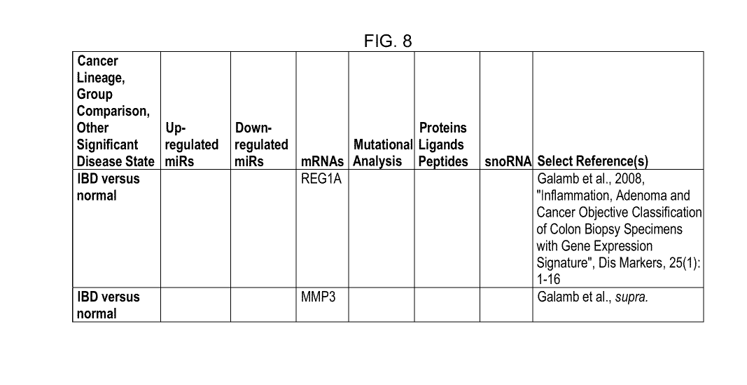

IBD versus normal biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 8, and can be used to create a IBD versus normal specific exosome bio-signature. For example, the one or more mRNAs that may be analyzed can include, but are not limited to, REG1A, MMP3, or any combination thereof.

Also provided herein is an isolated exosome comprising one or more specific biomarkers for distinguishing between IBD and a normal sample, such as listed in FIG. 8. A composition comprising the isolated exosome is also provided. Accordingly, in some embodiments, the composition comprises a population of exosomes comprising one or more specific biomarkers for distinguishing between IBD and a normal sample, such as listed in FIG. 8. The composition can comprise a substantially enriched population of exosomes, wherein the population of exosomes is substantially homogeneous for having one or more specific biomarkers for distinguishing between IBD and a normal sample, such as listed in FIG. 8.

One or more specific biomarkers for distinguishing between IBD and a normal sample, such as listed in FIG. 8 can also be detected by one or more systems disclosed herein, for distinguishing between IBD and a normal sample. For example, a detection system can comprise one or more probes to detect one or more specific biomarkers for distinguishing between IBD and a normal sample, such as listed in FIG. 8, of one or more exosomes of a biological sample.

Adenoma versus Colorectal Cancer (CRC)

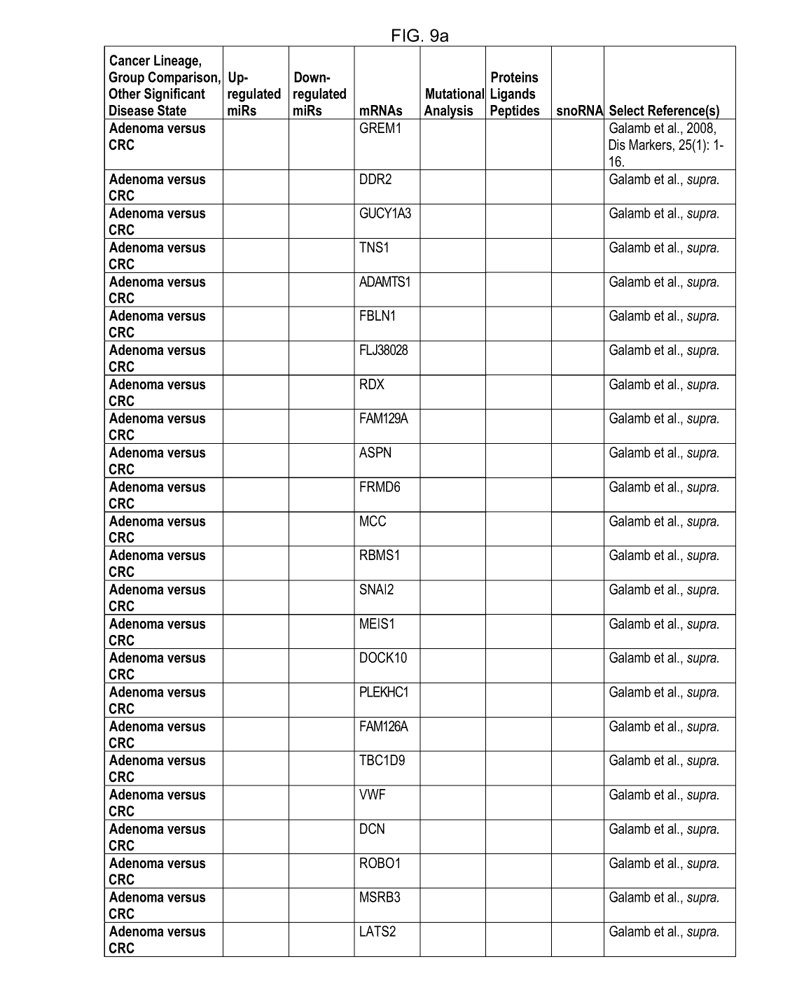

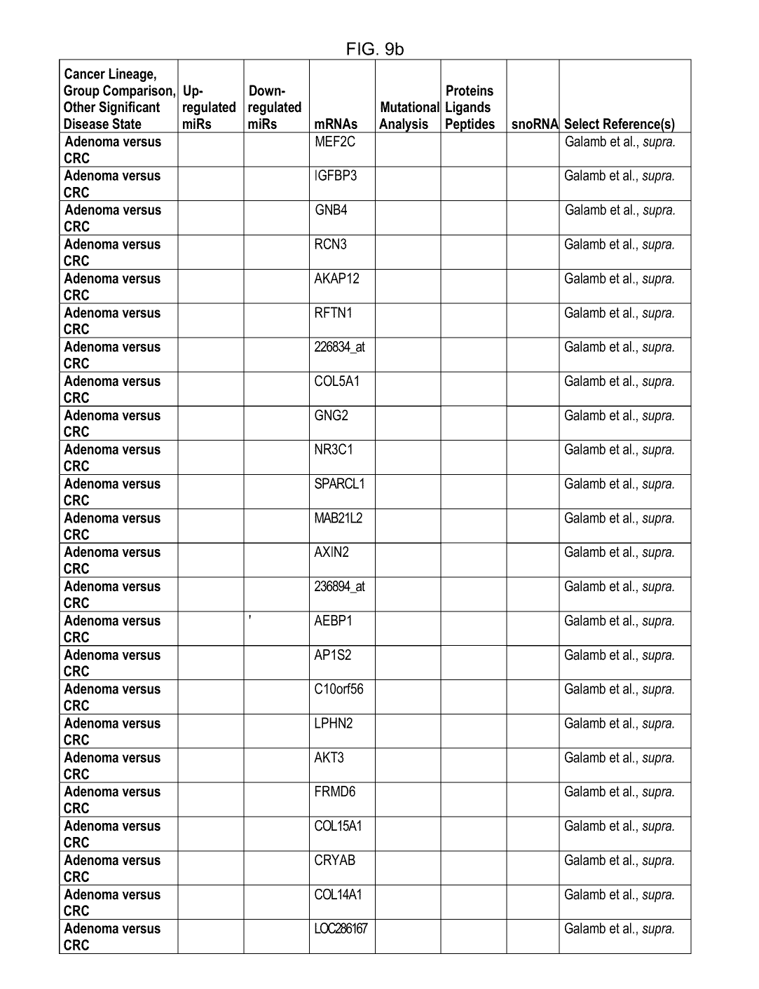

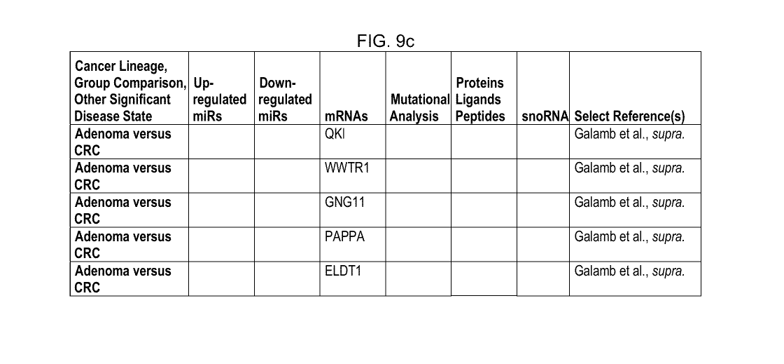

Adenoma versus CRC specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 9, and can be used to create a Adenoma versus CRC specific exosome bio-signature. For example, the one or more mRNAs that may be analyzed can include, but are not limited to, GREM1, DDR2, GUCY1A3, TNS1, ADAMTS1, FBLN1, FLJ38028, RDX, FAM129A, ASPN, FRMD6, MCC, RBMS1, SNAI2, MEIS1, DOCK10, PLEKHC1, FAM126A, TBC1D9, VWF, DCN, ROBO1, MSRB3, LATS2, MEF2C, IGFBP3, GNB4, RCN3, AKAP12, RFTN1, 226834 at, COL5A1, GNG2, NR3C1*, SPARCL1, MAB21L2, AXIN2, 236894 at, AEBP1, AP1S2, C10orf56, LPHN2, AKT3, FRMD6, COL15A1, CRYAB, COL14A1, LOC286167, QKI, WWTR1, GNG11, PAPPA, or ELDT1, or any combination thereof.

Also provided herein is an isolated exosome comprising one or more specific biomarkers for distinguishing between an adenoma and a CRC, such as listed in FIG. 9. A composition comprising the isolated exosome is also provided. Accordingly, in some embodiments, the composition comprises a population of exosomes comprising one or more specific biomarkers for distinguishing between an adenoma and a CRC, such as listed in FIG. 9. The composition can comprise a substantially enriched population of exosomes, wherein the population of exosomes is substantially homogeneous for having one or more specific biomarkers for distinguishing between an adenoma and a CRC, such as listed in FIG. 9.

One or more specific biomarkers for distinguishing between an adenoma and a CRC, such as listed in FIG. 9 can also be detected by one or more systems disclosed herein, for distinguishing between an adenoma and a CRC. For example, a detection system can comprise one or more probes to detect one or more specific biomarkers for distinguishing between an adenoma and a CRC, such as listed in FIG. 9, of one or more exosomes of a biological sample.

IBD Versus CRC

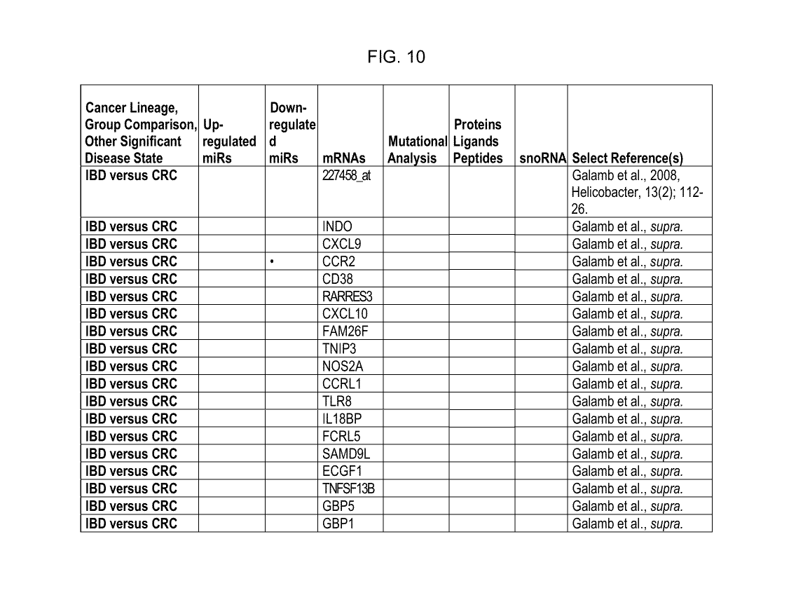

IBD versus CRC specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 10, and can be used to create a IBD versus CRC specific exosome bio-signature. For example, the one or more mRNAs that may be analyzed can include, but are not limited to, 227458 at, INDO, CXCL9, CCR2, CD38, RARRES3, CXCL10, FAM26F, TNIP3, NOS2A, CCRL1, TLR8, IL18BP, FCRLS, SAMD9L, ECGF1, TNFSF13B, GBP5, or GBP1, or any combination thereof.

Also provided herein is an isolated exosome comprising one or more specific biomarkers for distinguishing between IBD and a CRC, such as listed in FIG. 10. A composition comprising the isolated exosome is also provided. Accordingly, in some embodiments, the composition comprises a population of exosomes comprising one or more specific biomarkers for distinguishing between IBD and a CRC, such as listed in FIG. 10. The composition can comprise a substantially enriched population of exosomes, wherein the population of exosomes is substantially homogeneous for having one or more specific biomarkers for distinguishing between IBD and a CRC, such as listed in FIG. 10.

One or more specific biomarkers for distinguishing between IBD and a CRC, such as listed in FIG. 10 can also be detected by one or more systems disclosed herein, for distinguishing between IBD and a CRC. For example, a detection system can comprise one or more probes to detect one or more specific biomarkers for distinguishing between IBD and a CRC, such as listed in FIG. 10, of one or more exosomes of a biological sample.

CRC Dukes B Versus Dukes C-D

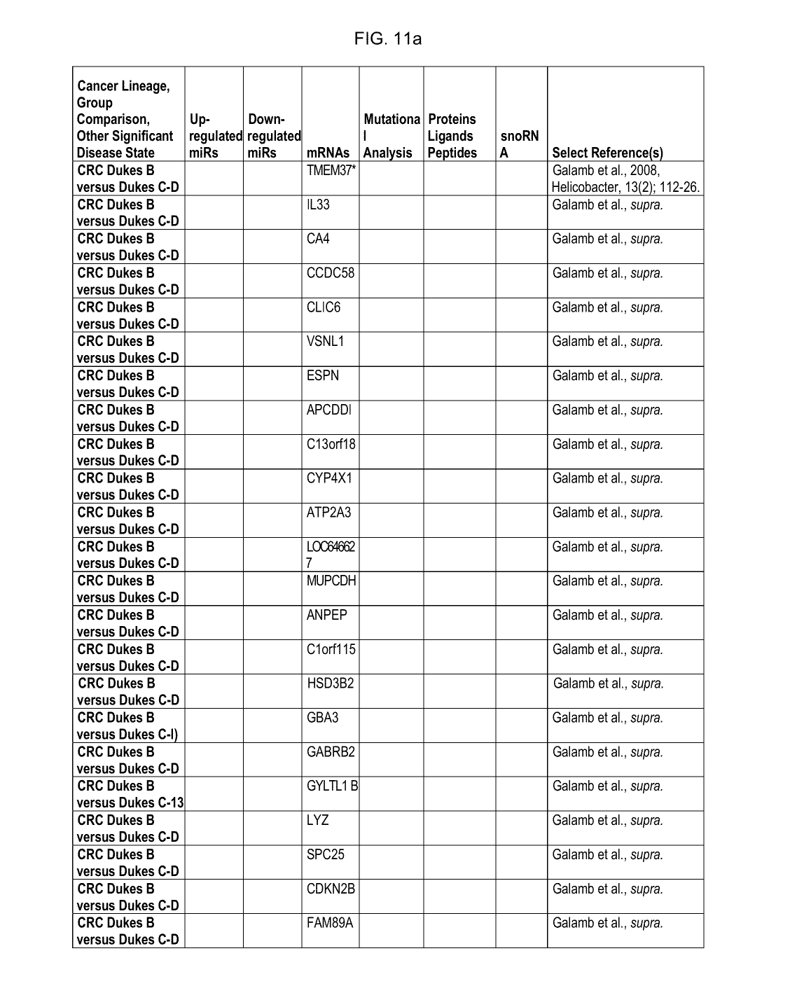

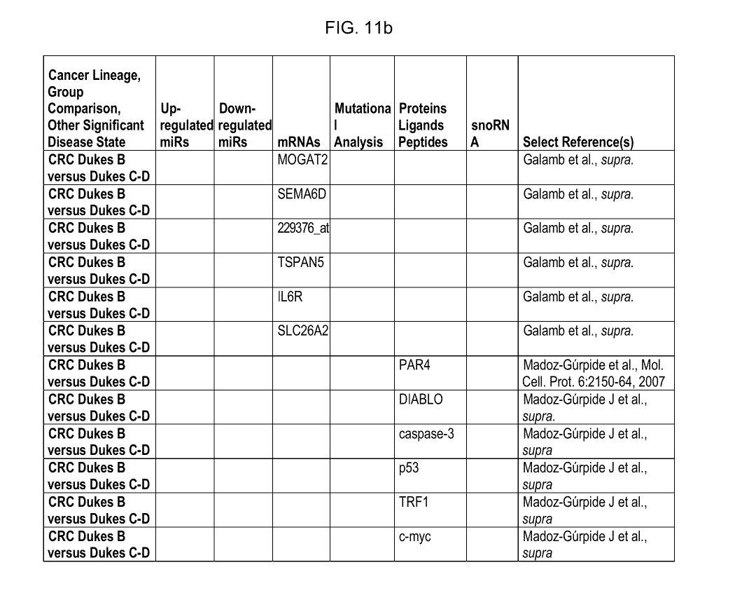

CRC Dukes B versus Dukes C-D specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 11, and can be used to create a CRC D-B versus C-D specific exosome bio-signature. For example, the one or more mRNAs that may be analyzed can include, but are not limited to, TMEM37*, IL33, CA4, CCDC58, CLIC6, VERSUSNL1, ESPN, APCDD1, C13orf18, CYP4X1, ATP2A3, LOC646627, MUPCDH, ANPEP, Clorf115, HSD3B2, GBA3, GABRB2, GYLTL1B, LYZ, SPC25, CDKN2B, FAM89A, MOGAT2, SEMA6D, 229376_at, TSPAN5, IL6R, or SLC26A2, or any combination thereof.

Also provided herein is an isolated exosome comprising one or more specific biomarkers for distinguishing between CRC Dukes B and a CRC Dukes C-D, such as listed in FIG. 11. A composition comprising the isolated exosome is also provided. Accordingly, in some embodiments, the composition comprises a population of exosomes comprising one or more specific biomarkers for distinguishing between CRC Dukes B and a CRC Dukes C-D, such as listed in FIG. 11. The composition can comprise a substantially enriched population of exosomes, wherein the population of exosomes is substantially homogeneous for having one or more specific biomarkers for distinguishing between CRC Dukes B and a CRC Dukes C-D, such as listed in FIG. 11.

One or more specific biomarkers for distinguishing between CRC Dukes B and a CRC Dukes C-D, such as listed in FIG. 11 can also be detected by one or more systems disclosed herein, for distinguishing between CRC Dukes B and a CRC Dukes C-D. For example, a detection system can comprise one or more probes to detect one or more specific biomarkers for distinguishing between CRC Dukes B and a CRC Dukes C-D, such as listed in FIG. 11, of one or more exosomes of a biological sample.

Adenoma with Low Grade Dysplasia Versus Adenoma with High Grade Dysplasia

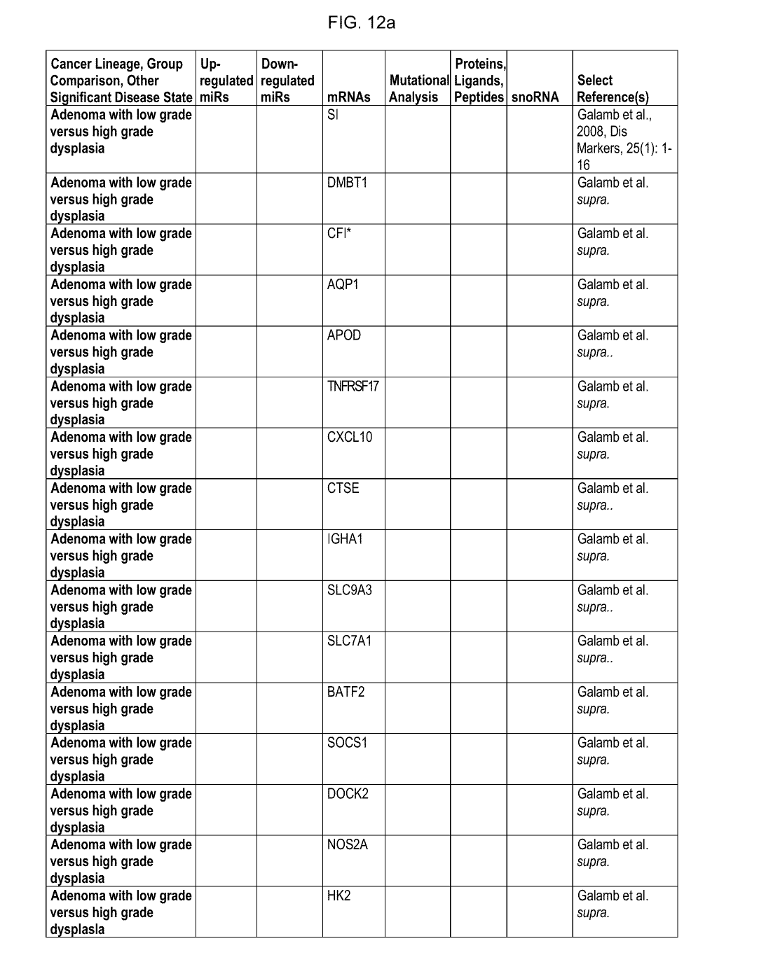

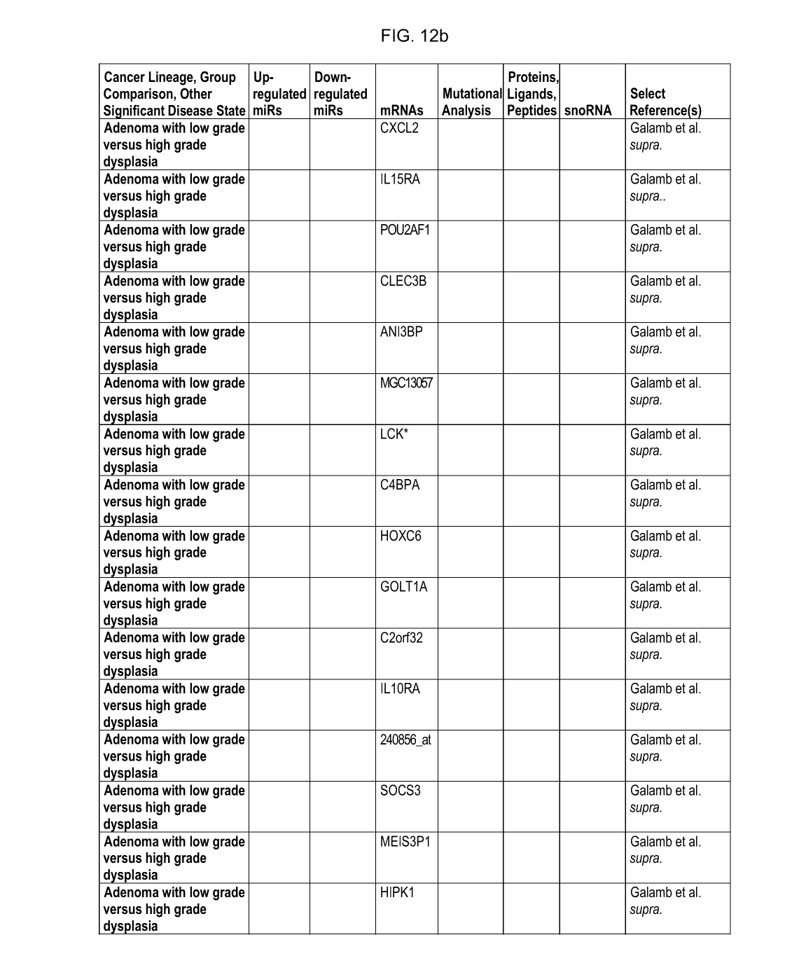

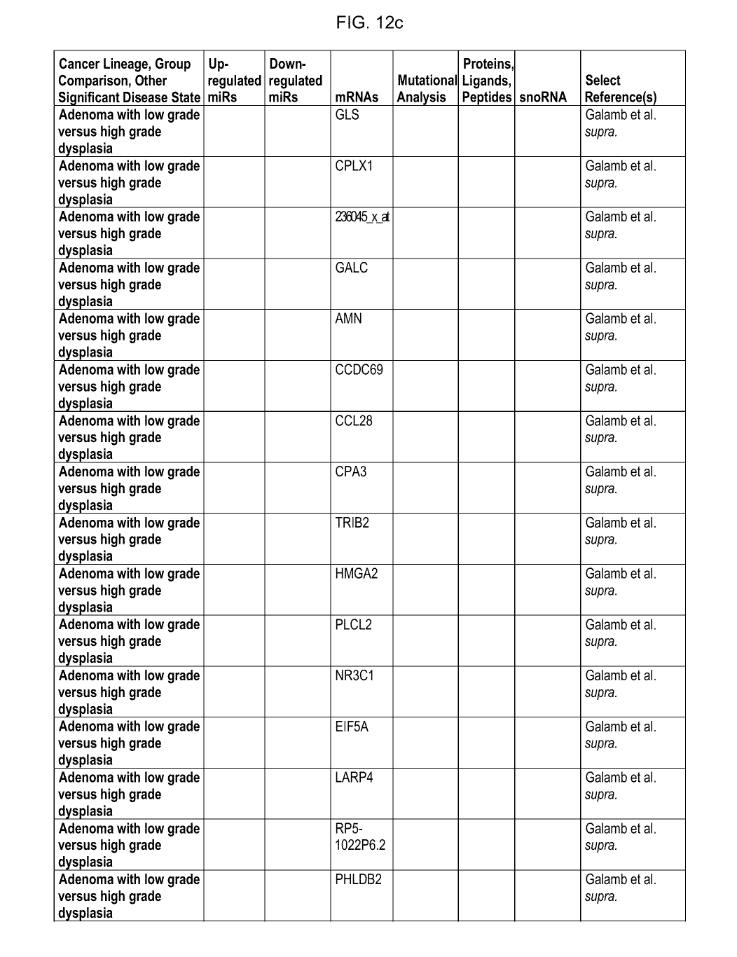

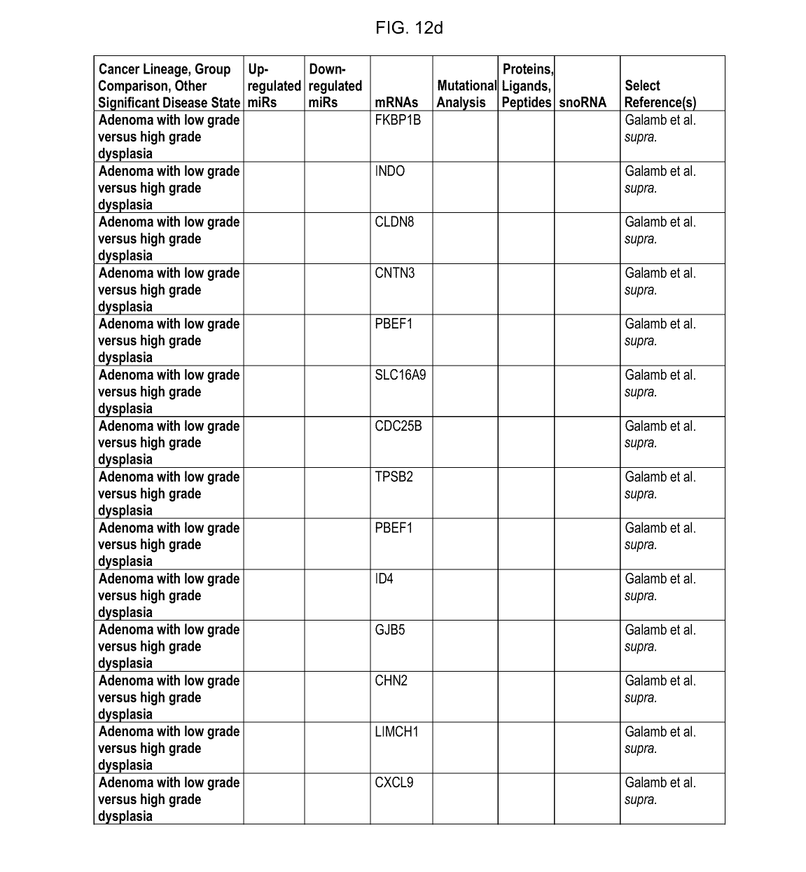

Adenoma with low grade dysplasia versus adenoma with high grade dysplasia specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 12, and can be used to create an adenoma low grade dysplasia versus adenoma high grade dysplasia specific exosome bio-signature. For example, the one or mRNAs that may be analyzed can include, but are not limited to, SI, DMBT1, CFI*, AQP1, APOD, TNFRSF17, CXCL10, CTSE, IGHA1, SLC9A3, SLC7A1, BATF2, SOCS1, DOCK2, NOS2A, HK2, CXCL2, IL15RA, POU2AF1, CLEC3B, ANI3BP, MGC13057, LCK*, C4BPA, HOXC6, GOLT1A, C2orf32, IL10RA, 240856 at, S0053, MEIS3P1, HIPK1, GLS, CPLX1, 236045_x_at, GALC, AMN, CCDC69, CCL28, CPA3, TRIB2, HMGA2, PLCL2, NR3C1, EIF5A, LARP4, RP5-1022P6.2, PHLDB2, FKBP1B, INDO, CLDN8, CNTN3, PBEF1, SLC16A9, CDC25B, TPSB2, PBEF1, ID4, GJB5, CHN2, LIMCH1, or CXCL9, or any combination thereof.

Also provided herein is an isolated exosome comprising one or more specific biomarkers for distinguishing between adenoma with low grade dysplasia and adenoma with high grade dysplasia, such as listed in FIG. 12. A composition comprising the isolated exosome is also provided. Accordingly, in some embodiments, the composition comprises a population of exosomes comprising one or more specific biomarkers for distinguishing between adenoma with low grade dysplasia and adenoma with high grade dysplasia, such as listed in FIG. 12. The composition can comprise a substantially enriched population of exosomes, wherein the population of exosomes is substantially homogeneous for having one or more specific biomarkers for distinguishing between adenoma with low grade dysplasia and adenoma with high grade dysplasia, such as listed in FIG. 12.

One or more specific biomarkers for distinguishing between adenoma with low grade dysplasia and adenoma with high grade dysplasia, such as listed in FIG. 12 can also be detected by one or more systems disclosed herein, for distinguishing between adenoma with low grade dysplasia and adenoma with high grade dysplasia. For example, a detection system can comprise one or more probes to detect one or more specific biomarkers for distinguishing between adenoma with low grade dysplasia and adenoma with high grade dysplasia, such as listed in FIG. 12, of one or more exosomes of a biological sample.

Ulcerative Colitis (UC) Versus Crohn's Disease (CD)

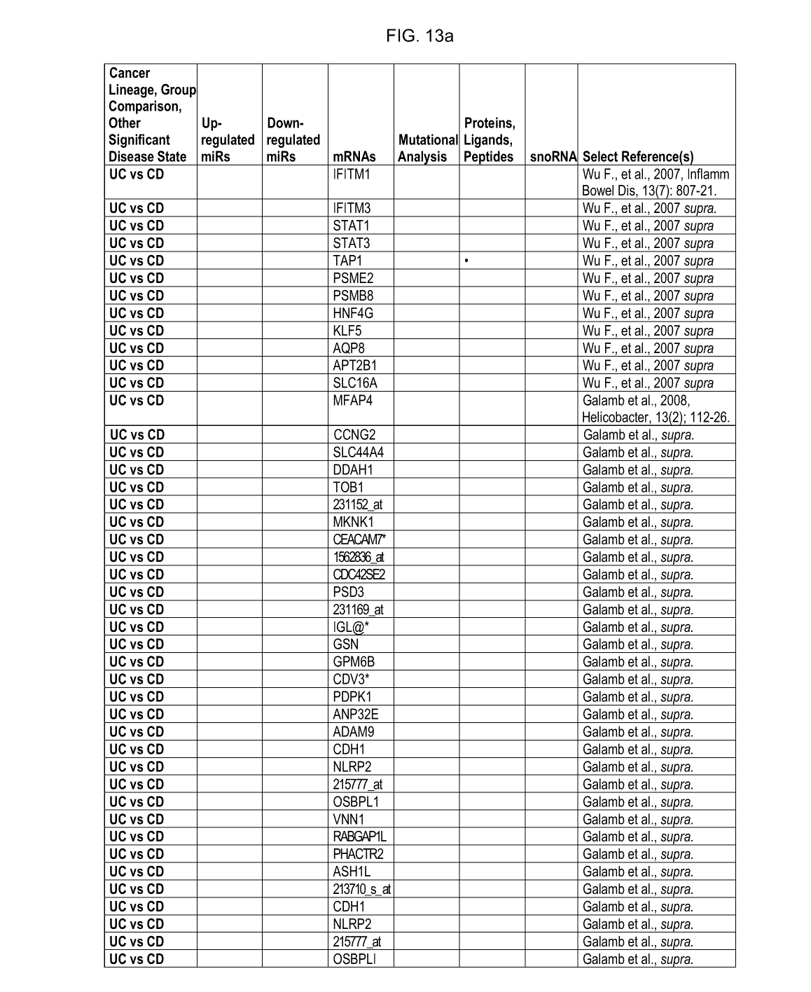

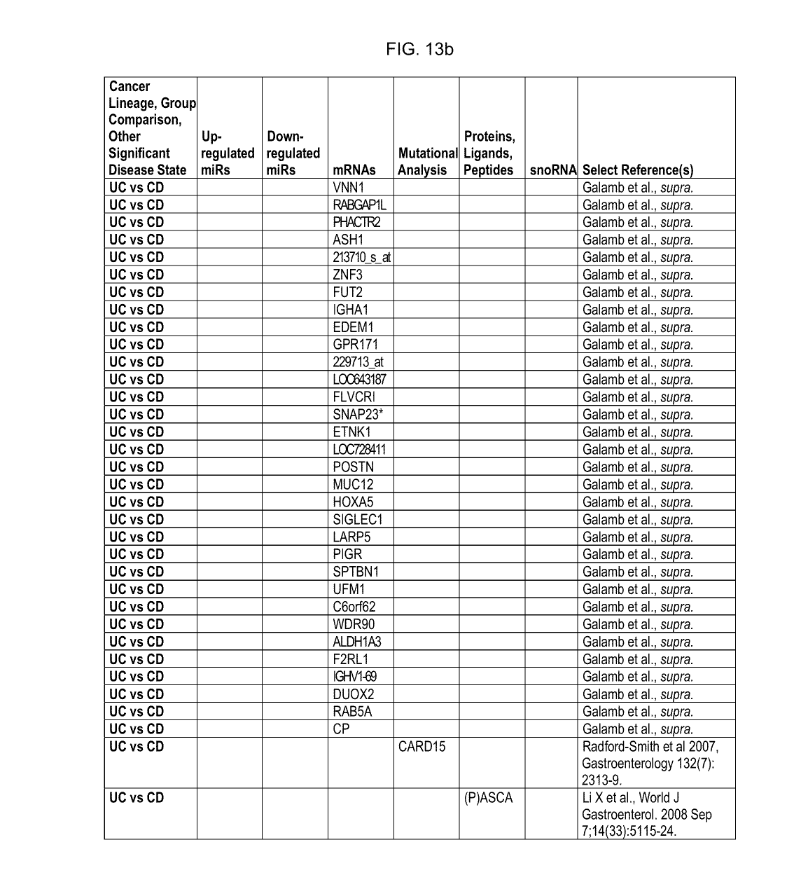

Ulcerative colitis (UC) versus Crohn's disease (CD) specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 13, and can be used to create a UC versus CD specific exosome bio-signature. For example, the one or more mRNAs that may be analyzed can include, but are not limited to, IFITM1, IFITM3, STAT1, STAT3, TAP1, PSME2, PSMB8, HNF4G, KLF5, AQP8, APT2B1, SLC16A, MFAP4, CCNG2, SLC44A4, DDAH1, TOB1, 231152_at, MKNK1, CEACAM7*, 1562836_at, CDC42SE2, PSD3, 231169_at, IGL@*, GSN, GPM6B, CDV3*, PDPK1, ANP32E, ADAMS, CDH1, NLRP2, 215777_at, OSBPL1, VNN1, RABGAP1L, PHACTR2, ASH1L, 213710_s_at, CDH1, NLRP2, 215777 at, OSBPL1, VNN1, RABGAP1L, PHACTR2, ASH1, 213710_s_at, ZNF3, FUT2, IGHA1, EDEM1, GPR171, 229713_at, LOC643187, FLVCR1, SNAP23*, ETNK1, LOC728411, POSTN, MUC12, HOXAS, SIGLEC1, LARP5, PIGR, SPTBN1, UFM1, C6orf62, WDR90, ALDH1A3, F2RL1, IGHV1-69, DUOX2, RAB5A, or CP, or any combination thereof can also be used as specific biomarkers from exosomes for UC versus CD.

A biomarker mutation for distinguishing UC versus CD that can be assessed in an exosome includes, but is not limited to, a mutation of CARD15, or any combination of mutations specific for distinguishing UC versus CD. The protein, ligand, or peptide that can be assessed in an exosome can include, but is not limited to, (P)ASCA.

Also provided herein is an isolated exosome comprising one or more specific biomarkers for distinguishing between UC and CD, such as listed in FIG. 13. A composition comprising the isolated exosome is also provided. Accordingly, in some embodiments, the composition comprises a population of exosomes comprising one or more specific biomarkers for distinguishing between UC and CD, such as listed in FIG. 13. The composition can comprise a substantially enriched population of exosomes, wherein the population of exosomes is substantially homogeneous for having one or more specific biomarkers for distinguishing between UC and CD, such as listed in FIG. 13.

One or more specific biomarkers for distinguishing between UC and CD, such as listed in FIG. 13 can also be detected by one or more systems disclosed herein, for distinguishing between UC and CD. For example, a detection system can comprise one or more probes to detect one or more specific biomarkers for distinguishing between UC and CD, such as listed in FIG. 13, of one or more exosomes of a biological sample.

Hyperplastic Polyp

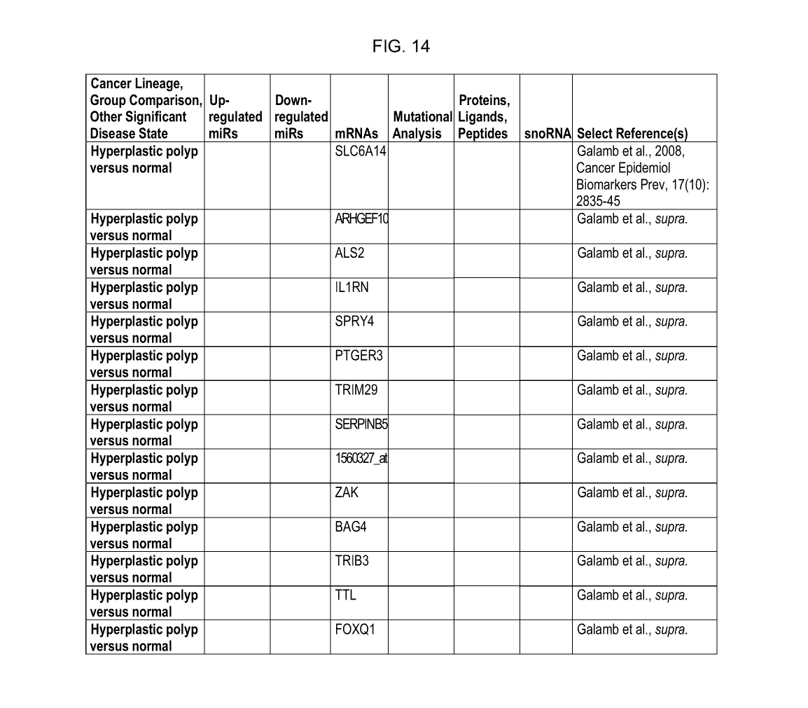

Hyperplastic polyp versus normal specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 14, and can be used to create a hyperplastic polyp versus normal specific exosome bio-signature. For example, the one or more mRNAs that may be analyzed can include, but are not limited to, SLC6A14, ARHGEF10, ALS2, IL1RN, SPRY4, PTGER3, TRIM29, SERPINB5, 1560327_at, ZAK, BAG4, TRIB3, TTL, FOXQ1, or any combination.

Also provided herein is an isolated exosome comprising one or more hyperplastic polyp specific biomarkers, such as listed in FIG. 14. A composition comprising the isolated exosome is also provided. Accordingly, in some embodiments, the composition comprises a population of exosomes comprising one or more hyperplastic polyp specific biomarkers, such as listed in FIG. 14. The composition can comprise a substantially enriched population of exosomes, wherein the population of exosomes is substantially homogeneous for hyperplastic polyp specific exosomes or exosomes comprising one or more hyperplastic polyp specific biomarkers, such as listed in FIG. 14.

One or more hyperplastic polyp specific biomarkers, such as listed in FIG. 14 can also be detected by one or more systems disclosed herein, for characterizing a hyperplastic polyp. For example, a detection system can comprise one or more probes to detect one or more listed in FIG. 14. One or more hyperplastic specific biomarkers, such as listed in FIG. 14, of one or more exosomes of a biological sample.

Adenoma with Low Grade Dysplasia Versus Normal

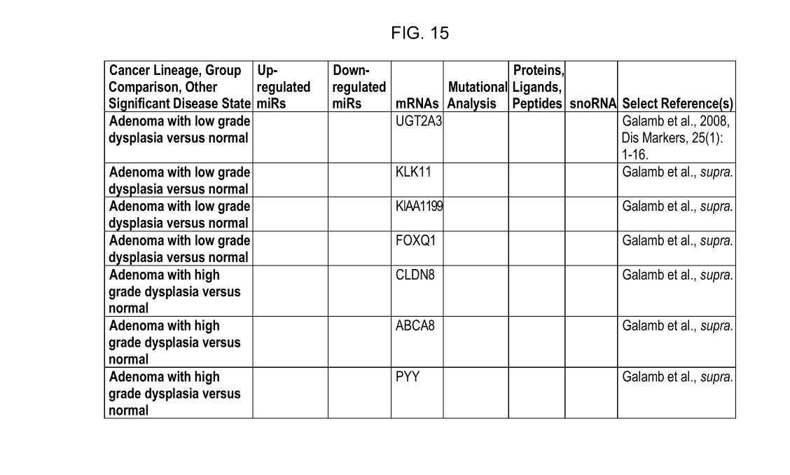

Adenoma with low grade dysplasia versus normal specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 15, and can be used to create an adenoma low grade dysplasia versus normal specific exosome bio-signature. For example, the RNAs that may be analyzed can include, but are not limited to, UGT2A3, KLK11, KIAA1199, FOXQ1, CLDN8, ABCA8, or PYY, or any combination thereof and can be used as specific biomarkers from exosomes for Adenoma low grade dysplasia versus normal. Furthermore, the snoRNA that can be used as an exosomal biomarker for adenoma low grade dysplasia versus normal can include, but is not limited to, GAS5.

Also provided herein is an isolated exosome comprising one or more specific biomarkers for distinguishing between adenoma with low grade dysplasia and normal, such as listed in FIG. 15. A composition comprising the isolated exosome is also provided. Accordingly, in some embodiments, the composition comprises a population of exosomes comprising one or more specific biomarkers for distinguishing between adenoma with low grade dysplasia and normal, such as listed in FIG. 15. The composition can comprise a substantially enriched population of exosomes, wherein the population of exosomes is substantially homogeneous for having one or more specific biomarkers for distinguishing between adenoma with low grade dysplasia and normal, such as listed in FIG. 15.

One or more specific biomarkers for distinguishing between adenoma with low grade dysplasia and normal, such as listed in FIG. 15 can also be detected by one or more systems disclosed herein, for distinguishing between adenoma with low grade dysplasia and normal. For example, a detection system can comprise one or more probes to detect one or more specific biomarkers for distinguishing between adenoma with low grade dysplasia and normal, such as listed in FIG. 15, of one or more exosomes of a biological sample.

Adenoma Versus Normal

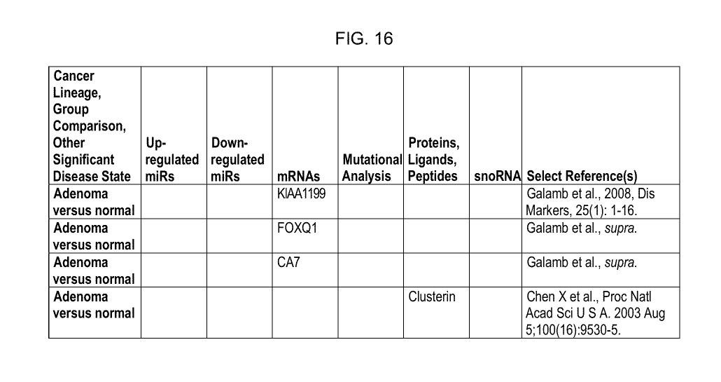

Adenoma versus normal specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 16, and can be used to create an Adenoma versus normal specific exosome bio-signature. For example, the one or more mRNAs that may be analyzed can include, but are not limited to, KIAA1199, FOXQ1, or CA7, or any combination thereof. The protein, ligand, or peptide that can be used as a biomarker from exosomes that is specific to adenoma versus. normal can include, but is not limited to, Clusterin.

Also provided herein is an isolated exosome comprising one or more specific biomarkers for distinguishing between adenoma and normal, such as listed in FIG. 16. A composition comprising the isolated exosome is also provided. Accordingly, in some embodiments, the composition comprises a population of exosomes comprising one or more specific biomarkers for distinguishing between adenoma and normal, such as listed in FIG. 16. The composition can comprise a substantially enriched population of exosomes, wherein the population of exosomes is substantially homogeneous for having one or more specific biomarkers for distinguishing between adenoma and normal, such as listed in FIG. 16.

One or more specific biomarkers for distinguishing between adenoma and normal, such as listed in FIG. 16 can also be detected by one or more systems disclosed herein, for distinguishing between adenoma and normal. For example, a detection system can comprise one or more probes to detect one or more specific biomarkers for distinguishing between adenoma and normal, such as listed in FIG. 16, of one or more exosomes of a biological sample.

CRC Versus Normal

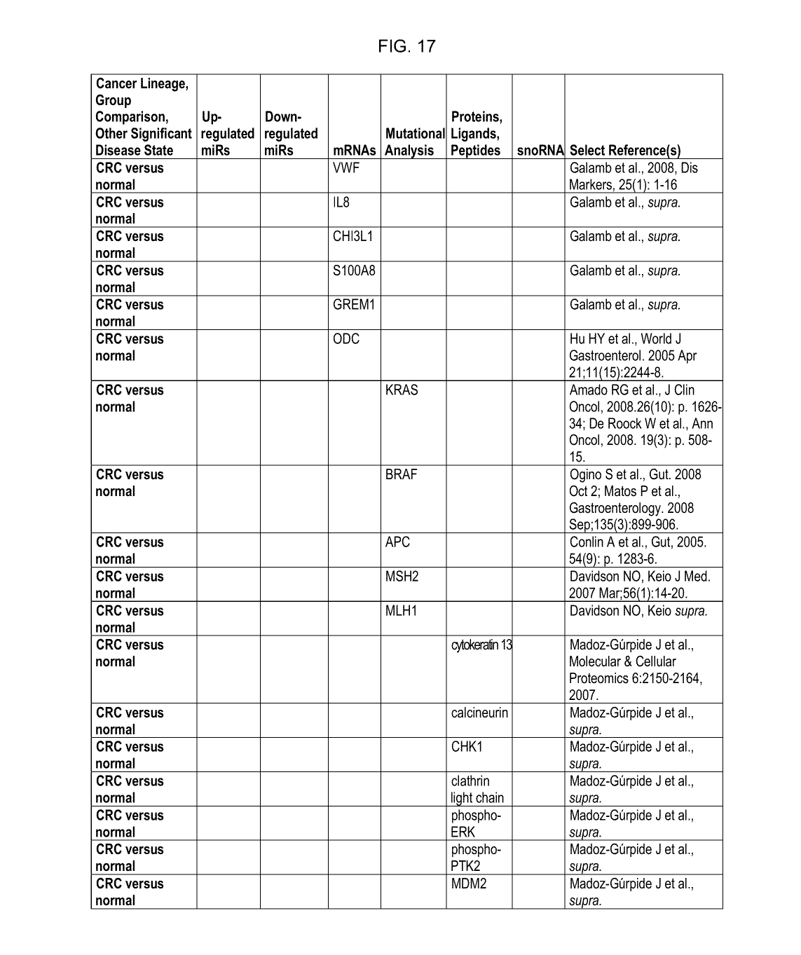

CRC versus normal specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 17, and can be used to create a CRC versus normal specific exosome bio-signature. For example, the one or mRNAs that may be analyzed can include, but are not limited to, VWF, IL8, CHI3L1, S100A8, GREM1, or ODC, or any combination thereof and can be used as specific biomarkers from exosomes for CRC versus normal.

A biomarker mutation for CRC versus normal that can be assessed in an exosome includes, but is not limited to, a mutation of KRAS, BRAF, APC, MSH2, or MLH1, or any combination of mutations specific for distinguishing between CRC versus normal. The protein, ligand, or peptide that can be assessed in an exosome can include, but is not limited to, cytokeratin 13, calcineurin, CHK1, clathrin light chain, phospho-ERK, phospho-PTK2, or MDM2, or any combination thereof.

Also provided herein is an isolated exosome comprising one or more specific biomarkers for distinguishing between CRC and normal, such as listed in FIG. 17. A composition comprising the isolated exosome is also provided. Accordingly, in some embodiments, the composition comprises a population of exosomes comprising one or more specific biomarkers for distinguishing between CRC and normal, such as listed in FIG. 17. The composition can comprise a substantially enriched population of exosomes, wherein the population of exosomes is substantially homogeneous for having one or more specific biomarkers for distinguishing between CRC and normal, such as listed in FIG. 17.

One or more specific biomarkers for distinguishing between CRC and normal, such as listed in FIG. 17 can also be detected by one or more systems disclosed herein, for distinguishing between CRC and normal. For example, a detection system can comprise one or more probes to detect one or more specific biomarkers for distinguishing between CRC and normal, such as listed in FIG. 17, of one or more exosomes of a biological sample.

Benign Prostatic Hyperplasia (BPH)

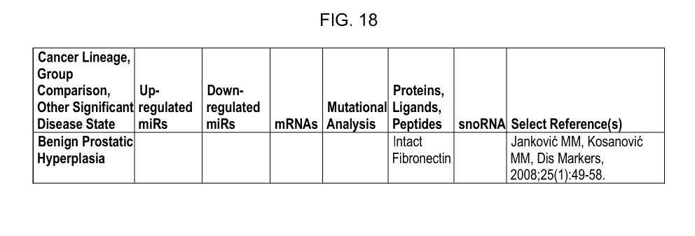

Benign prostatic hyperplasia (BPH) specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 18, and can be used to create a BPH specific exosome bio-signature. The protein, ligand, or peptide that can be assessed in an exosome can include, but is not limited to, intact fibronectin.

Also provided herein is an isolated exosome comprising one or more BPH specific biomarkers, such as listed in FIG. 18 and in FIG. 1 for BPH. A composition comprising the isolated exosome is also provided. Accordingly, in some embodiments, the composition comprises a population of exosomes comprising one or more BPH specific biomarkers, such as listed in FIG. 18 and in FIG. 1 for BPH. The composition can comprise a substantially enriched population of exosomes, wherein the population of exosomes is substantially homogeneous for BPH specific exosomes or exosomes comprising one or more BPH specific biomarkers, such as listed in FIG. 18 and in FIG. 1 for BPH.

One or more BPH specific biomarkers, such as listed in FIG. 18 and in FIG. 1 for BPH, can also be detected by one or more systems disclosed herein, for characterizing a BPH. For example, a detection system can comprise one or more probes to detect one or more BPH specific biomarkers, such as listed in FIG. 18 and in FIG. 1 for BPH, of one or more exosomes of a biological sample.

Prostate Cancer

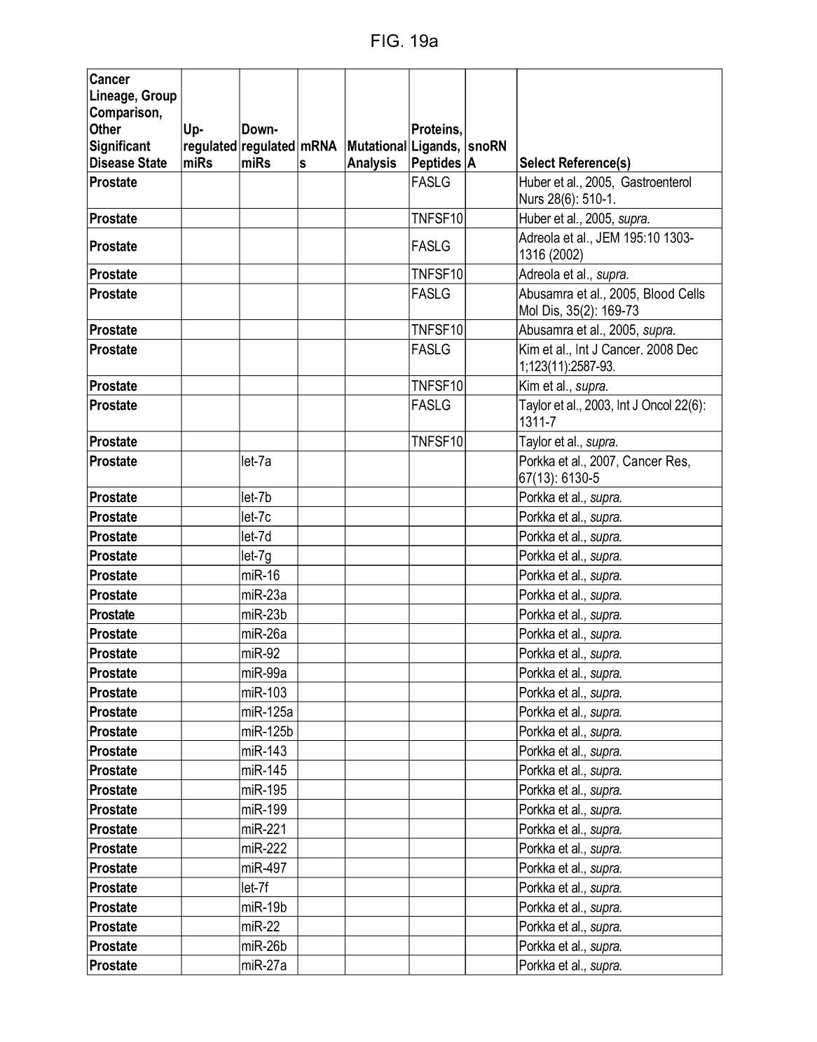

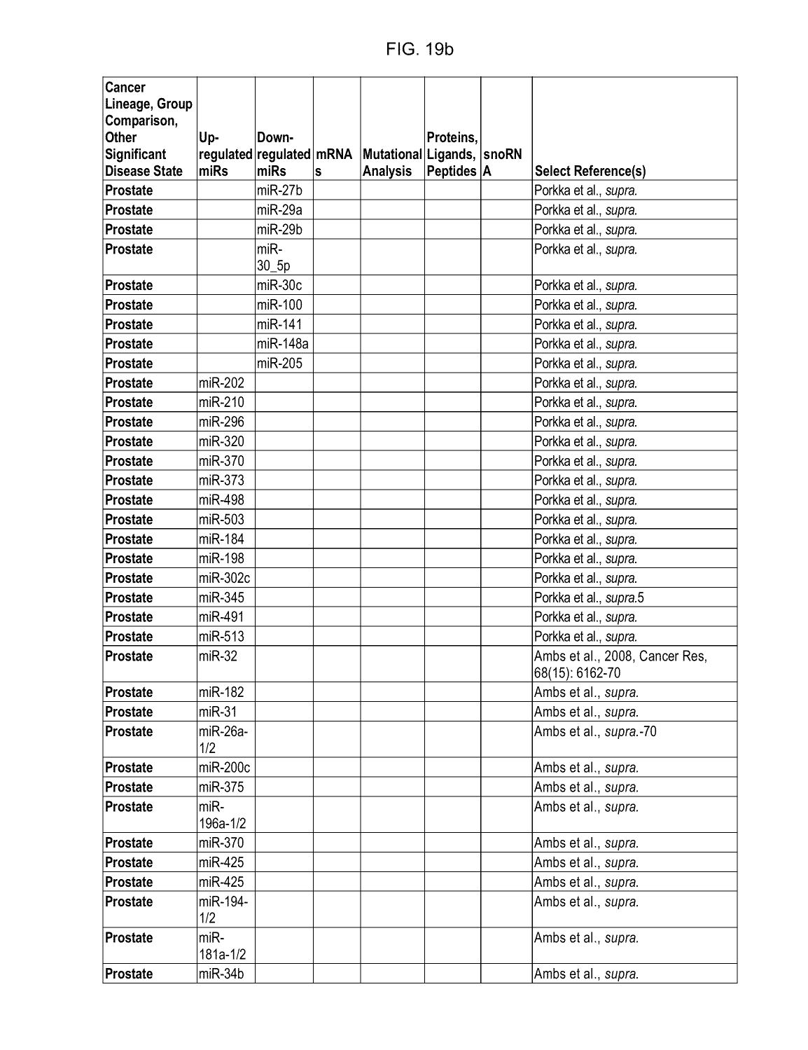

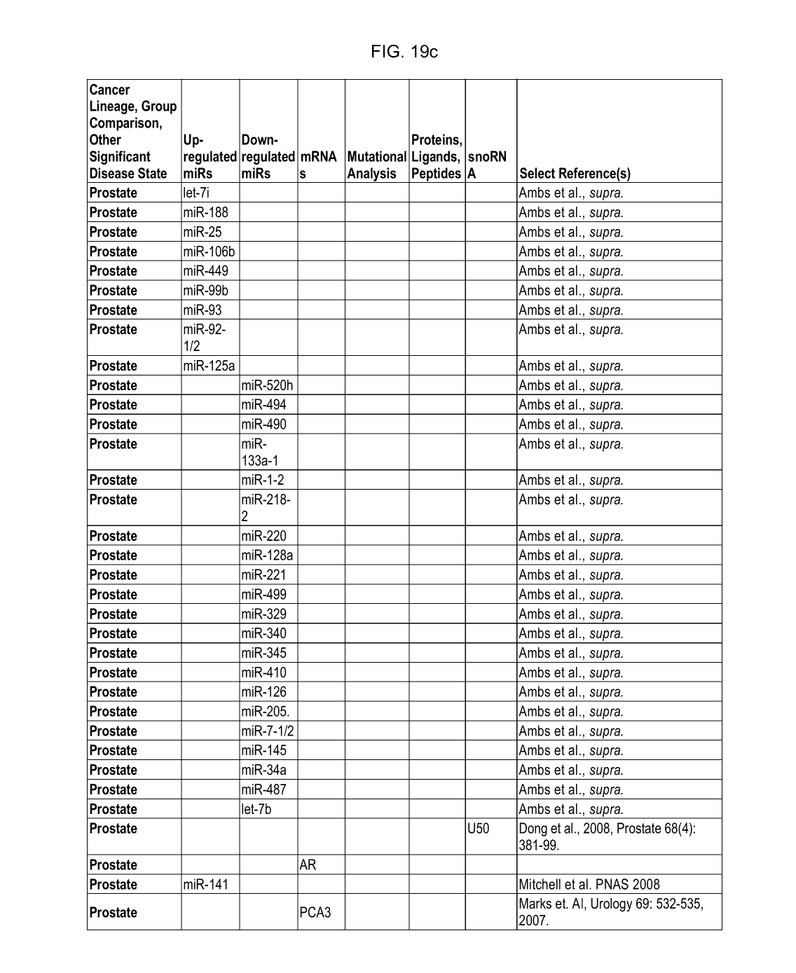

Prostate cancer specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 19, and can be used to create a prostate cancer specific exosome bio-signature. For example, a bio-signature for prostate cancer can comprise miR-9, miR-21, miR-141, miR-370, miR-200b, miR-210, miR-155, or miR-196a. In some embodiments, the bio-signature can comprise one or more overexpressed miRs, such as, but not limited to, miR-202, miR-210, miR-296, miR-320, miR-370, miR-373, miR-498, miR-503, miR-184, miR-198, miR-302c, miR-345, miR-491, miR-513, miR-32, miR-182, miR-31, miR-26a-1/2, miR-200c, miR-375, miR-196a-1/2, miR-370, miR-425, miR-425, miR-194-1/2, miR-181a-1/2, miR-34b, let-7i, miR-188, miR-25, miR-106b, miR-449, miR-99b, miR-93, miR-92-1/2, miR-125a, or miR-141, or any combination thereof.

The bio-signature can also comprise one or more underexpressed miRs such as, but not limited to, let-7a, let-7b, let-7c, let-7d, let-7g, miR-16, miR-23a, miR-23b, miR-26a, miR-92, miR-99a, miR-103, miR-125a, miR-125b, miR-143, miR-145, miR-195, miR-199, miR-221, miR-222, miR-497, let-7f, miR-19b, miR-22, miR-26b, miR-27a, miR-27b, miR-29a, miR-29b, miR-30_{—}5p, miR-30c, miR-100, miR-141, miR-148a, miR-205, miR-520h, miR-494, miR-490, miR-133a-1, miR-1-2, miR-218-2, miR-220, miR-128a, miR-221, miR-499, miR-329, miR-340, miR-345, miR-410, miR-126, miR-205, miR-7-1/2, miR-145, miR-34a, miR-487, or let-7b, or any combination thereof. The bio-signature can comprise upregulated or overexpressed miR-21, downregulated or underexpressed miR-15a, miR-16-1, miR-143 or miR-145, or any combination thereof

The one or more mRNAs that may be analyzed can include, but are not limited to, AR, PCA3, or any combination thereof and can be used as specific biomarkers from exosomes for prostate cancer.

The protein, ligand, or peptide that can be assessed in an exosome can include, but is not limited to, FASLG or TNFSF10 or any combination thereof. Furthermore, an exosome isolated or assayed can be prostate cancer cell specific, or derived from prostate cancer cells. Furthermore, the snoRNA that can be used as an exosomal biomarker for prostate cancer can include, but is not limited to, U50. Examples of prostate cancer bio-signatures are further described below.

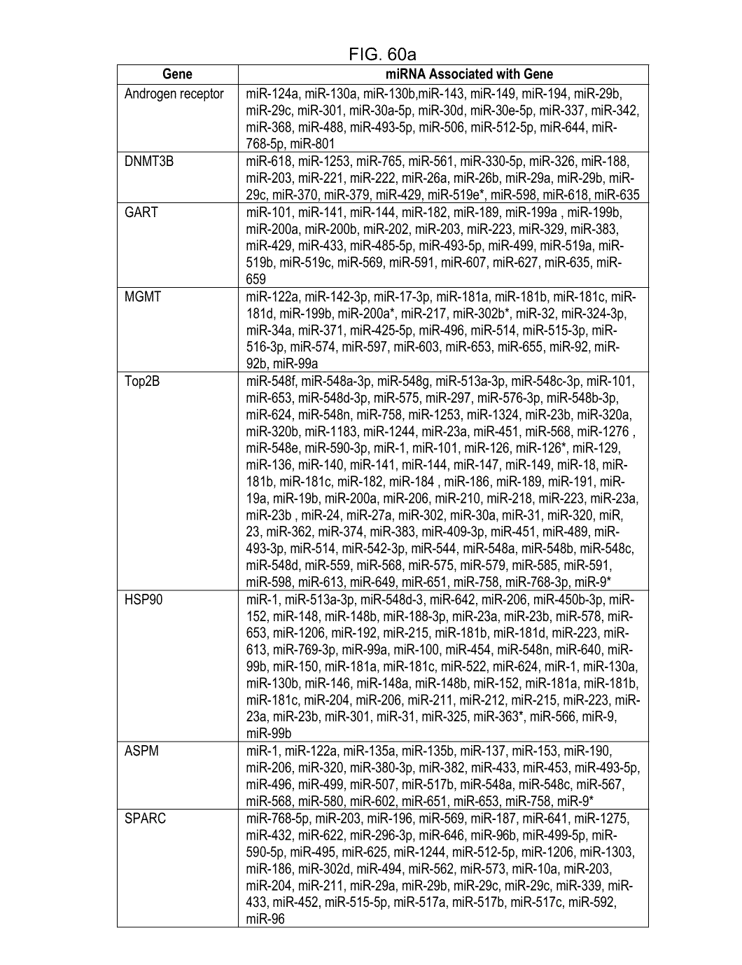

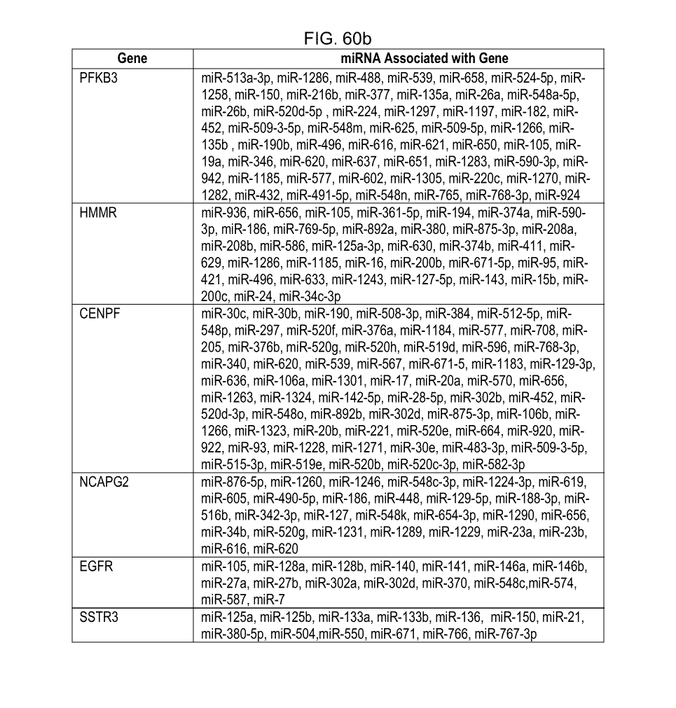

Also provided herein is an isolated exosome comprising one or more prostate cancer specific biomarkers, such as ACSL3-ETV1, C150RF21-ETV1, F1135294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4, or those listed in FIGS. 19, 60 and in FIG. 1 for prostate cancer. A composition comprising the isolated exosome is also provided. Accordingly, in some embodiments, the composition comprises a population of exosomes comprising one or more prostate cancer specific biomarkers such as ACSL3-ETV1, C150RF21-ETV1, F1135294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4, or those listed in FIGS. 19, 60 and in FIG. 1 for prostate cancer. The composition can comprise a substantially enriched population of exosomes, wherein the population of exosomes is substantially homogeneous for prostate cancer specific exosomes or exosomes comprising one or more prostate cancer specific biomarkers, such as ACSL3-ETV1, C150RF21-ETV1, F1135294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4, or those listed in FIGS. 19, 60 and in FIG. 1 for prostate cancer.

One or more prostate cancer specific biomarkers, such as ACSL3-ETV1, C150RF21-ETV1, F1135294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4, or those listed in FIGS. 19, 60 and in FIG. 1 for prostate cancer can also be detected by one or more systems disclosed herein, for characterizing a prostate cancer. For example, a detection system can comprise one or more probes to detect one or more prostate cancer specific biomarkers, such as ACSL3-ETV1, C150RF21-ETV1, F1135294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4, or those listed in FIGS. 19, 60 and in FIG. 1 for prostate cancer, of one or more exosomes of a biological sample.

Melanoma

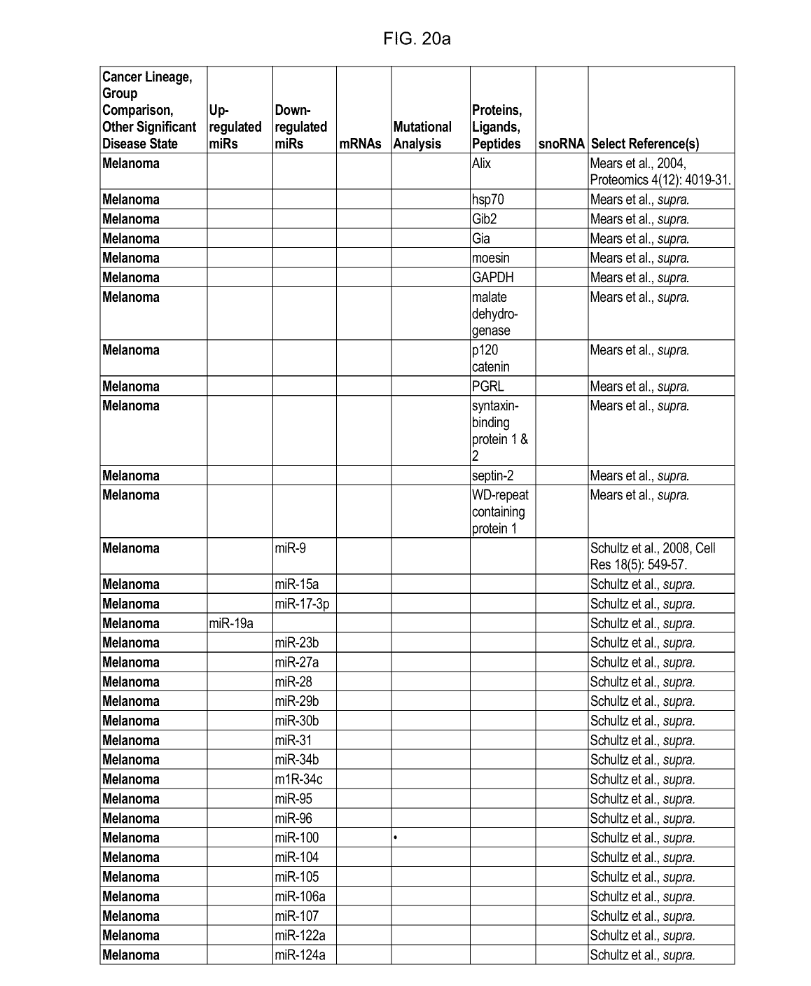

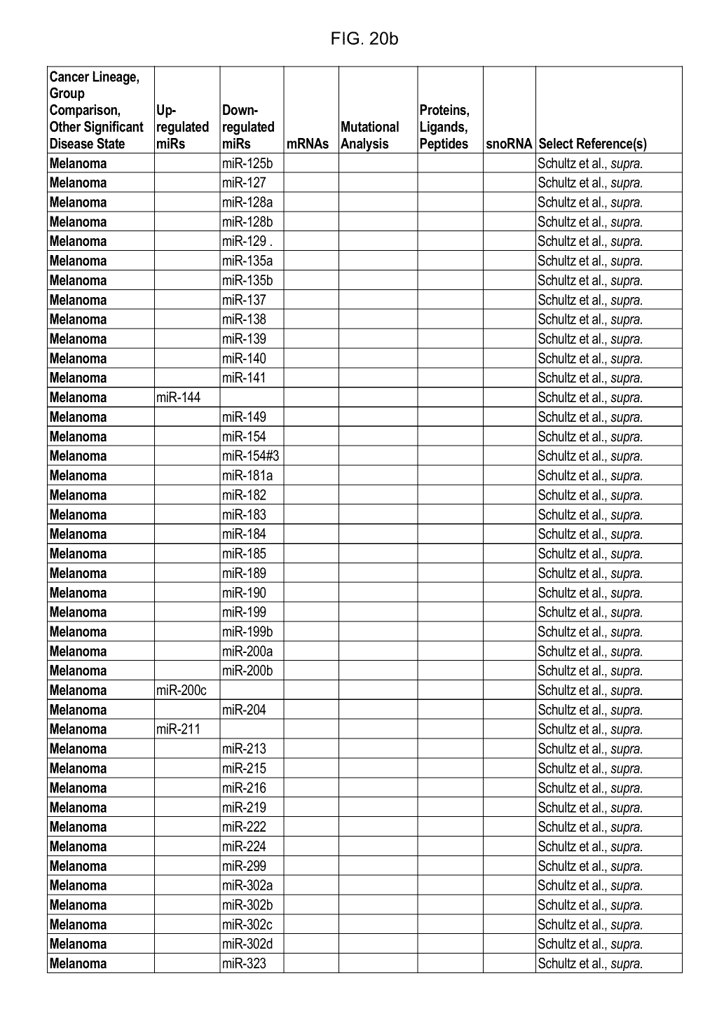

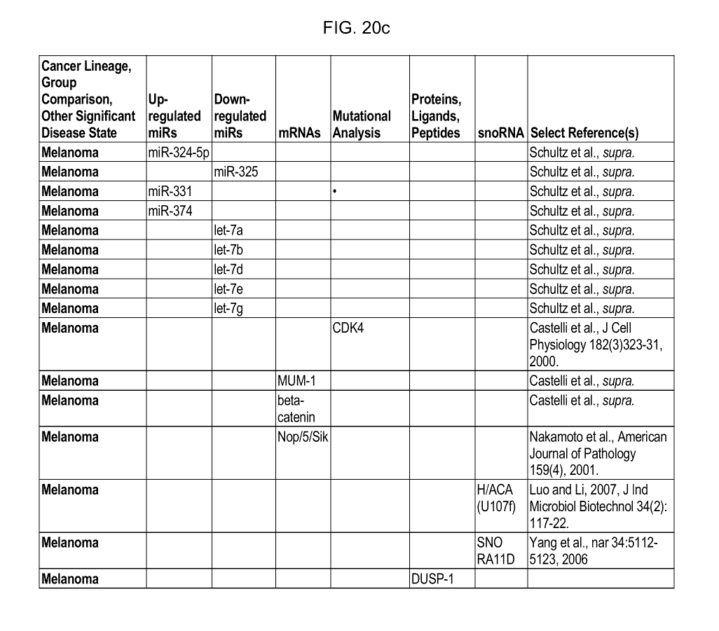

Melanoma specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 20, and can be used to create a melanoma specific exosome bio-signature. For example, the bio-signature can comprise one or more overexpressed miRs, such as, but not limited to, miR-19a, miR-144, miR-200c, miR-211, miR-324-5p, miR-331, or miR-374, or any combination thereof. The bio-signature can also comprise one or more underexpressed miRs such as, but not limited to, miR-9, miR-15a, miR-17-3p, miR-23b, miR-27a, miR-28, miR-29b, miR-30b, miR-31, miR-34b, miR-34c, miR-95, miR-96, miR-100, miR-104, miR-105, miR-106a, miR-107, miR-122a, miR-124a, miR-125b, miR-127, miR-128a, miR-128b, miR-129, miR-135a, miR-135b, miR-137, miR-138, miR-139, miR-140, miR-141, miR-149, miR-154, miR-154#3, miR-181a, miR-182, miR-183, miR-184, miR-185, miR-189, miR-190, miR-199, miR-199b, miR-200a, miR-200b, miR-204, miR-213, miR-215, miR-216, miR-219, miR-222, miR-224, miR-299, miR-302a, miR-302b, miR-302c, miR-302d, miR-323, miR-325, let-7a, let-7b, let-7d, let-7e, or let-7g, or any combination thereof.

The one or more mRNAs that may be analyzed can include, but are not limited to, MUM-1, beta-catenin, or Nop/5/Sik, or any combination thereof and can be used as specific biomarkers from exosomes for melanoma.

A biomarker mutation for melanoma that can be assessed in an exosome includes, but is not limited to, a mutation of CDK4 or any combination of mutations specific for melanoma. The protein, ligand, or peptide that can be assessed in an exosome can include, but is not limited to, DUSP-1, Alix, hsp70, Gib2, Gia, moesin, GAPDH, malate dehydrogenase, p120 catenin, PGRL, syntaxin-binding protein 1 & 2, septin-2, or WD-repeat containing protein 1, or any combination thereof. The snoRNA that can be used as an exosomal biomarker for melanoma include, but are not limited to, H/ACA (U107f), SNORA11D, or any combination thereof. Furthermore, an exosome isolated or assayed can be melanoma cell specific, or derived from melanoma cells.

Also provided herein is an isolated exosome comprising one or more melanoma specific biomarkers, such as listed in FIG. 20 and in FIG. 1 for melanoma. A composition comprising the isolated exosome is also provided. Accordingly, in some embodiments, the composition comprises a population of exosomes comprising one or more melanoma specific biomarkers, such as listed in FIG. 20 and in FIG. 1 for melanoma. The composition can comprise a substantially enriched population of exosomes, wherein the population of exosomes is substantially homogeneous for melanoma specific exosomes or exosomes comprising one or more melanoma specific biomarkers, such as listed in FIG. 20 and in FIG. 1 for melanoma.

One or more melanoma specific biomarkers, such as listed in FIG. 20 and in FIG. 1 for melanoma can also be detected by one or more systems disclosed herein, for characterizing a melanoma. For example, a detection system can comprise one or more probes to detect one or more cancer specific biomarkers, such as listed in FIG. 20 and in FIG. 1 for melanoma, of one or more exosomes of a biological sample.

Pancreatic Cancer

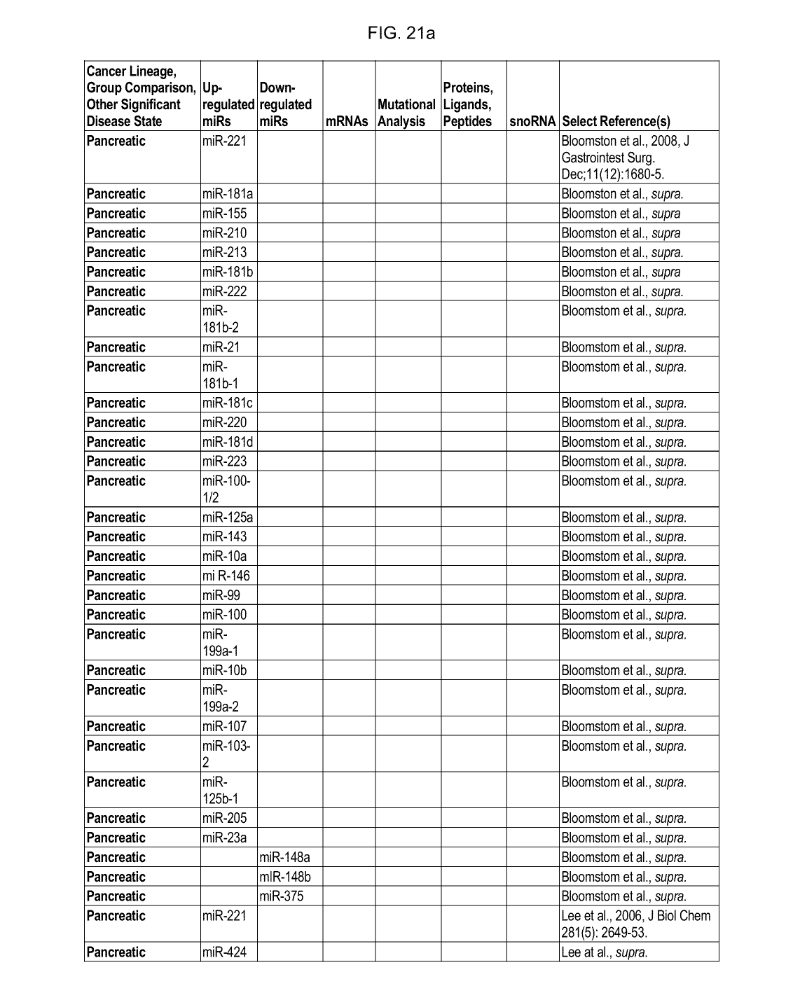

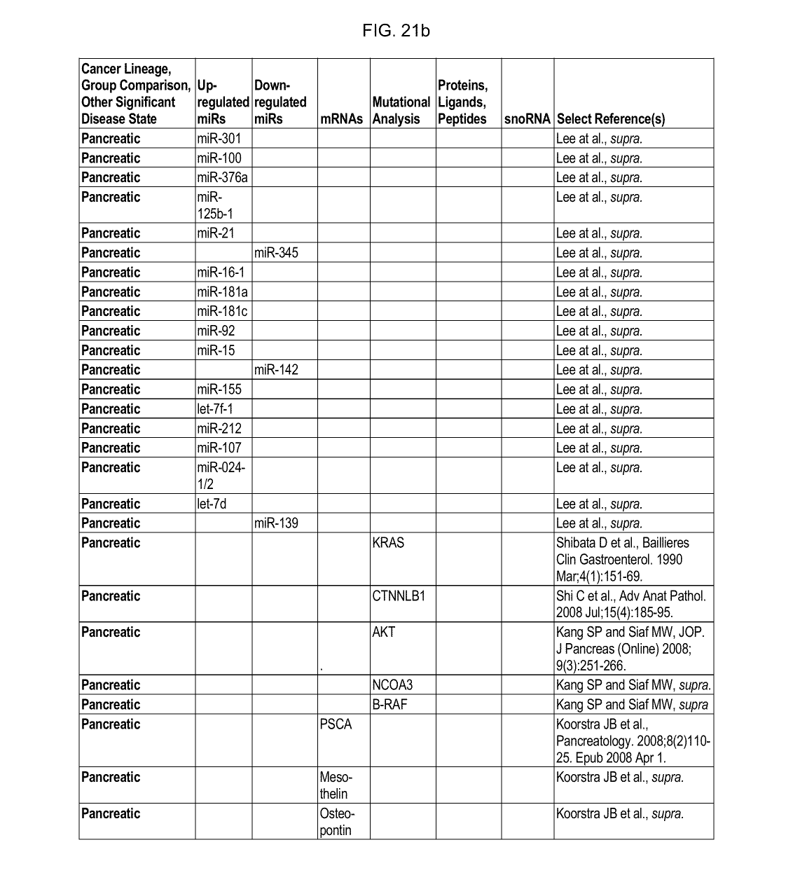

Pancreatic cancer specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 21, and can be used to create a pancreatic cancer specific exosome bio-signature. For example, the bio-signature can comprise one or more overexpressed miRs, such as, but not limited to, miR-221, miR-181a, miR-155, miR-210, miR-213, miR-181b, miR-222, miR-181b-2, miR-21, miR-181b-1, miR-220, miR-181d, miR-223, miR-100-1/2, miR-125a, miR-143, miR-10a, miR-146, miR-99, miR-100, miR-199a-1, miR-10b, miR-199a-2, miR-221, miR-181a, miR-155, miR-210, miR-213, miR-181b, miR-222, miR-181b-2, miR-21, miR-181b-1, miR-181c, miR-220, miR-181d, miR-223, miR-100-1/2, miR-125a, miR-143, miR-10a, miR-146, miR-99, miR-100, miR-199a-1, miR-10b, miR-199a-2, miR-107, miR-103, miR-103-2, miR-125b-1, miR-205, miR-23a, miR-221, miR-424, miR-301, miR-100, miR-376a, miR-125b-1, miR-21, miR-16-1, miR-181a, miR-181c, miR-92, miR-15, miR-155, let-7f-1, miR-212, miR-107, miR-024-1/2, miR-18a, miR-31, miR-93, miR-224, or let-7d, or any combination thereof.

The bio-signature can also comprise one or more underexpressed miRs such as, but not limited to, miR-148a, miR-148b, miR-375, miR-345, miR-142, miR-133a, miR-216, miR-217 or miR-139, or any combination thereof. The one or more mRNAs that may be analyzed can include, but are not limited to, PSCA, Mesothelin, or Osteopontin, or any combination thereof and can be used as specific biomarkers from exosomes for pancreatic cancer.

A biomarker mutation for pancreatic cancer that can be assessed in an exosome includes, but is not limited to, a mutation of KRAS, CTNNLB1, AKT, NCOA3, or B-RAF, or any combination of mutations specific for pancreatic cancer. The biomarker can also be BRCA2, PALB2, or p16. Furthermore, an exosome isolated or assayed can be pancreatic cancer cell specific, or derived from pancreatic cancer cells.

Also provided herein is an isolated exosome comprising one or more pancreatic cancer specific biomarkers, such as listed in FIG. 21. A composition comprising the isolated exosome is also provided. Accordingly, in some embodiments, the composition comprises a population of exosomes comprising one or more pancreatic cancer specific biomarkers, such as listed in FIG. 21. The composition can comprise a substantially enriched population of exosomes, wherein the population of exosomes is substantially homogeneous for pancreatic cancer specific exosomes or exosomes comprising one or more pancreatic cancer specific biomarkers, such as listed in FIG. 21.

One or more pancreatic cancer specific biomarkers, such as listed in FIG. 21, can also be detected by one or more systems disclosed herein, for characterizing a pancreatic cancer. For example, a detection system can comprise one or more probes to detect one or more pancreatic cancer specific biomarkers, such as listed in FIG. 21, of one or more exosomes of a biological sample.

Brain Cancer

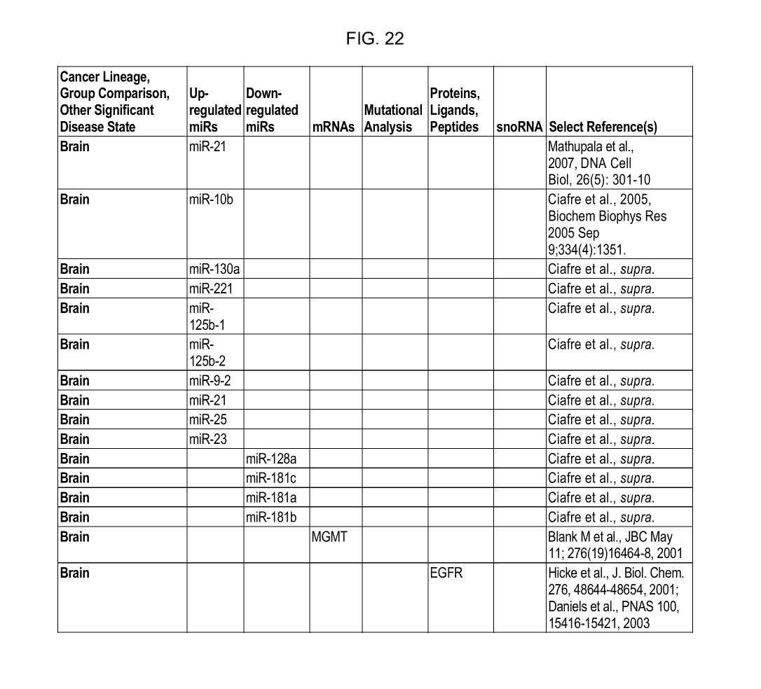

Brain cancer (including, but not limited to, gliomas, glioblastomas, meinigiomas, acoustic neuroma/schwannomas, medulloblastoma) specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 22, and can be used to create a brain cancer specific exosome bio-signature. For example, the bio-signature can comprise one or more overexpressed miRs, such as, but not limited to miR-21, miR-10b, miR-130a, miR-221, miR-125b-1, miR-125b-2, miR-9-2, miR-21, miR-25, or miR-123, or any combination thereof.

The bio-signature can also comprise one or more underexpressed miRs such as, but not limited to, miR-128a, miR-181c, miR-181a, or miR-181b, or any combination thereof. The one or more mRNAs that may be analyzed include, but are not limited to, MGMT, which can be used as specific biomarker from exosomes for brain cancer. The protein, ligand, or peptide that can be assessed in an exosome can include, but is not limited to, EGFR.

Also provided herein is an isolated exosome comprising one or more brain cancer specific biomarkers, such as GOPC-ROS1, or those listed in FIG. 22 and in FIG. 1 for brain cancer. A composition comprising the isolated exosome is also provided. Accordingly, in some embodiments, the composition comprises a population of exosomes comprising one or more brain cancer specific biomarkers, such as GOPC-ROS1, or those listed in FIG. 22 and in FIG. 1 for brain cancer. The composition can comprise a substantially enriched population of exosomes, wherein the population of exosomes is substantially homogeneous for brain cancer specific exosomes or exosomes comprising one or more brain cancer specific biomarkers, such as GOPC-ROS1, or those listed in FIG. 22. and in FIG. 1 for brain cancer.

One or more brain cancer specific biomarkers, such as listed in FIG. 22 and in FIG. 1 for brain cancer, can also be detected by one or more systems disclosed herein, for characterizing a brain cancer. For example, a detection system can comprise one or more probes to detect one or more brain cancer specific biomarkers, such as GOPC-ROS1, or those listed in FIG. 22 and in FIG. 1 for brain cancer, of one or more exosomes of a biological sample.

Psoriasis

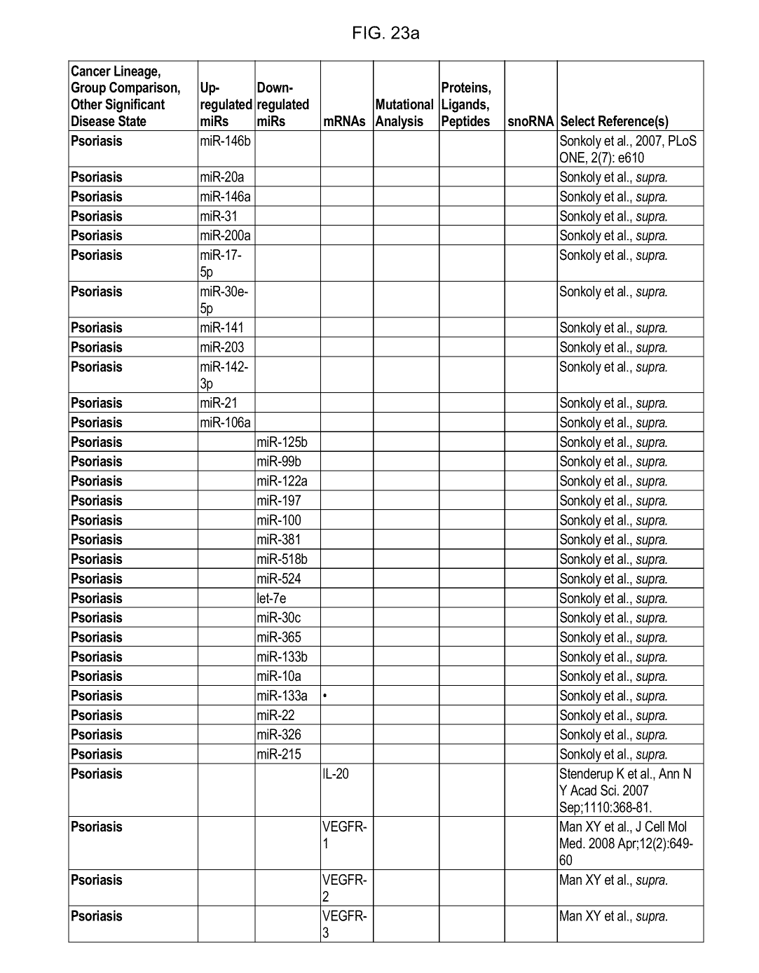

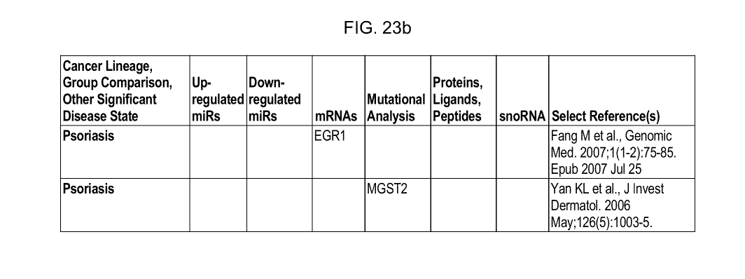

Psoriasis specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 23, and can be used to create a psoriasis specific exosome bio-signature. For example, the bio-signature can comprise one or more overexpressed miRs, such as, but not limited to, miR-146b, miR-20a, miR-146a, miR-31, miR-200a, miR-17-5p, miR-30e-5p, miR-141, miR-203, miR-142-3p, miR-21, or miR-106a, or any combination thereof. The bio-signature can also comprise one or more underexpressed miRs such a, but not limited to, miR-125b, miR-99b, miR-122a, miR-197, miR-100, miR-381, miR-518b, miR-524, let-7e, miR-30c, miR-365, miR-133b, miR-10a, miR-133a, miR-22, miR-326, or miR-215, or any combination thereof.

The one or more mRNAs that may be analyzed can include, but are not limited to, IL-20, VEGFR-1, VEGFR-2, VEGFR-3, or EGR1, or any combination thereof and can be used as specific biomarkers from exosomes for psoriasis. A biomarker mutation for psoriasis that can be assessed in an exosome includes, but is not limited to, a mutation of MGST2, or any combination of mutations specific for psoriasis.

Also provided herein is an isolated exosome comprising one or more psoriasis specific biomarkers, such as listed in FIG. 23 and in FIG. 1 for psoriasis. A composition comprising the isolated exosome is also provided. Accordingly, in some embodiments, the composition comprises a population of exosomes comprising one or more psoriasis specific biomarkers, such as listed in FIG. 23 and in FIG. 1 for psoriasis. The composition can comprise a substantially enriched population of exosomes, wherein the population of exosomes is substantially homogeneous for psoriasis specific exosomes or exosomes comprising one or more psoriasis specific biomarkers, such as listed in FIG. 23 and in FIG. 1 for psoriasis.

One or more psoriasis specific biomarkers, such as listed in FIG. 23 and in FIG. 1 for psoriasis, can also be detected by one or more systems disclosed herein, for characterizing psoriasis. For example, a detection system can comprise one or more probes to detect one or more psoriasis specific biomarkers, such as listed in FIG. 23 and in FIG. 1 for psoriasis, of one or more exosomes of a biological sample.

Cardiovascular Disease (CVD)

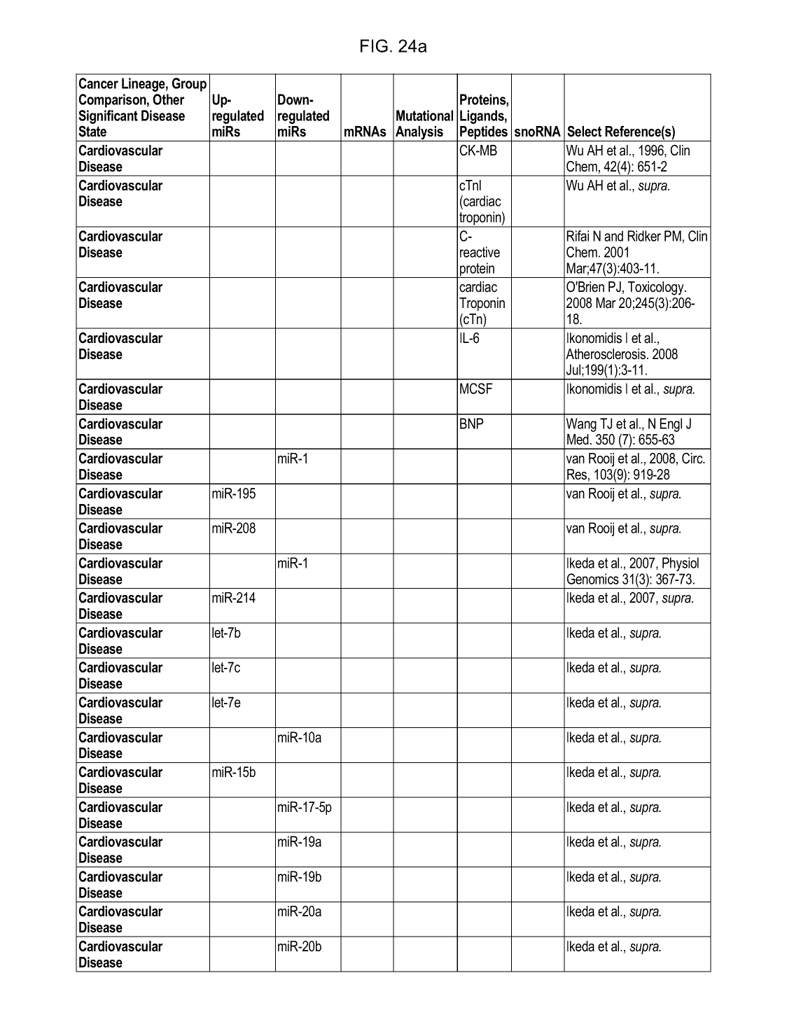

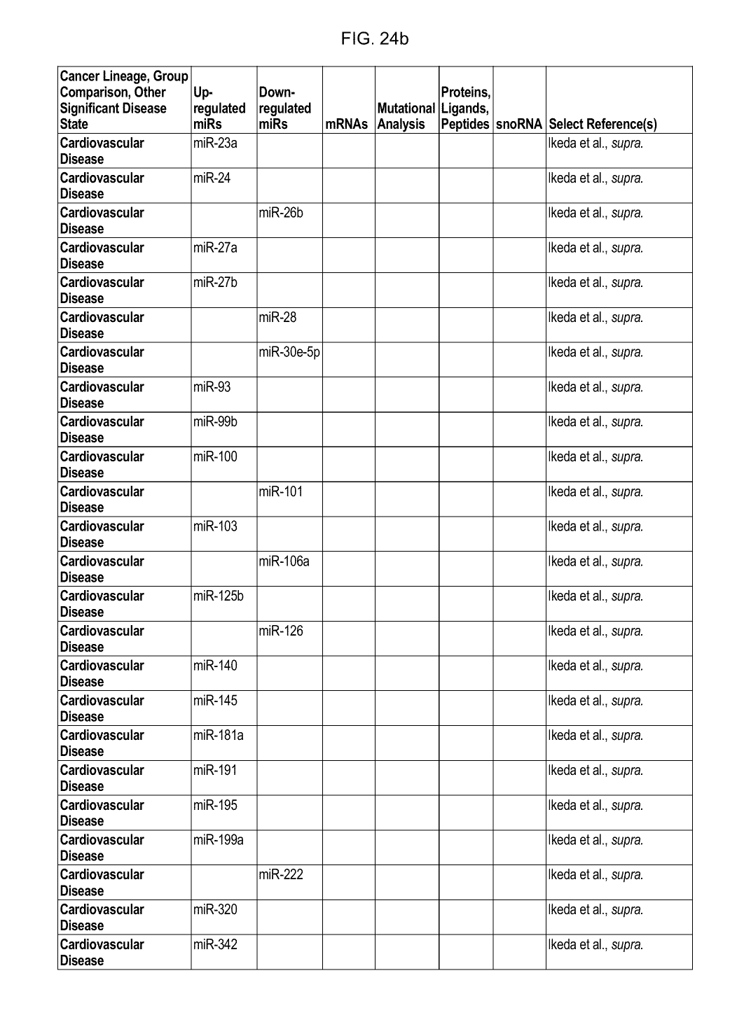

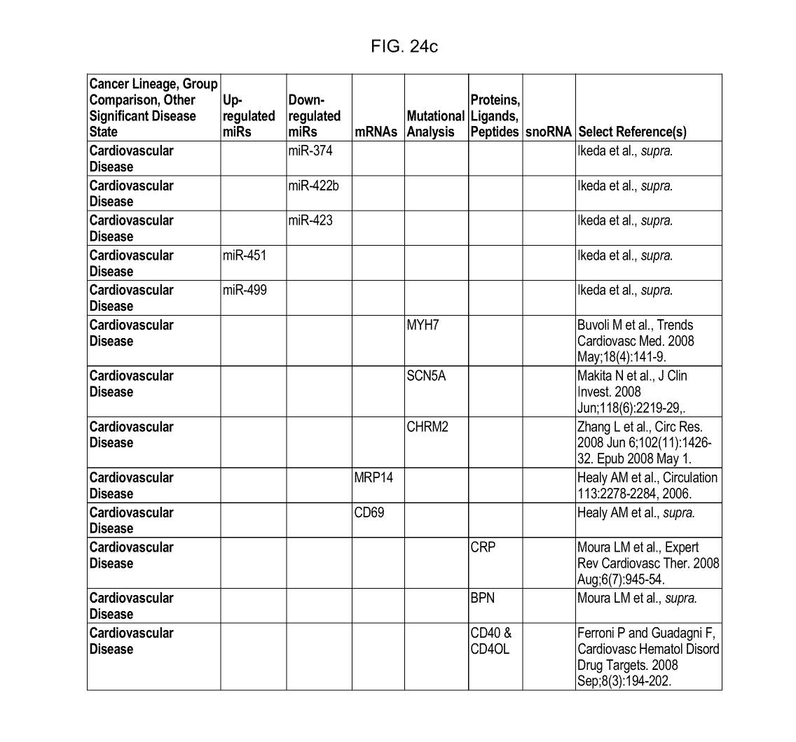

CVD specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 24, and can be used to create a CVD specific exosome bio-signature. For example, the bio-signature can comprise one or more overexpressed miRs, such as, but not limited to, miR-195, miR-208, miR-214, let-7b, let-7c, let-7e, miR-15b, miR-23a, miR-24, miR-27a, miR-27b, miR-93, miR-99b, miR-100, miR-103, miR-125b, miR-140, miR-145, miR-181a, miR-191, miR-195, miR-199a, miR-320, miR-342, miR-451, or miR-499, or any combination thereof.

The bio-signature can also comprise one or more underexpressed miRs such as, but not limited to, miR-1, miR-10a, miR-17-5p, miR-19a, miR-19b, miR-20a, miR-20b, miR-26b, miR-28, miR-30e-5p, miR-101, miR-106a, miR-126, miR-222, miR-374, miR-422b, or miR-423, or any combination thereof. The mRNAs that may be analyzed can include, but are not limited to, MRP14, CD69, or any combination thereof and can be used as specific biomarkers from exosomes for CVD.

A biomarker mutation for CVD that can be assessed in an exosome includes, but is not limited to, a mutation of MYH7, SCN5A, or CHRM2, or any combination of mutations specific for CVD.

The protein, ligand, or peptide that can be assessed in an exosome can include, but is not limited to, CK-MB, cTnI (cardiac troponin), CRP, BPN, IL-6, MCSF, CD40, CD40L, or any combination thereof. Furthermore, an exosome isolated or assayed can be a CVD cell specific, or derived from cardiac cells.

Also provided herein is an isolated exosome comprising one or more CVD specific biomarkers, such as listed in FIG. 24 and in FIG. 1 for CVD. A composition comprising the isolated exosome is also provided. Accordingly, in some embodiments, the composition comprises a population of exosomes comprising one or more CVD specific biomarkers, such as listed in FIG. 24 and in FIG. 1 for CVD. The composition can comprise a substantially enriched population of exosomes, wherein the population of exosomes is substantially homogeneous for CVD specific exosomes or exosomes comprising one or more CVD specific biomarkers, such as listed in FIG. 24 and in FIG. 1 for CVD.

One or more CVD specific biomarkers, such as listed in FIG. 24 and in FIG. 1 for CVD, can also be detected by one or more systems disclosed herein, for characterizing a CVD. For example, a detection system can comprise one or more probes to detect one or more CVD specific biomarkers, such as listed in FIG. 24 and in FIG. 1 for CVD, of one or more exosomes of a biological sample.

Blood Cancers

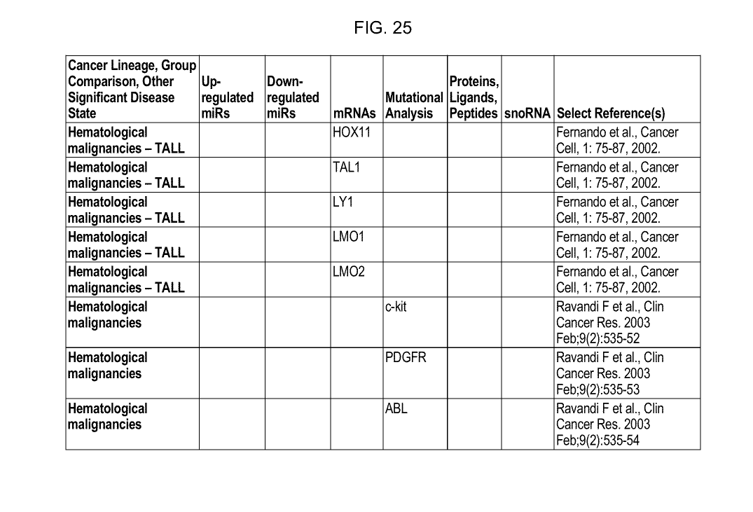

Hematological malignancies specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 25, and can be used to create a hematological malignancies specific exosome bio-signature. For example, the one or more mRNAs that may be analyzed can include, but are not limited to, HOX11, TAL1, LY1, LMO1, or LMO2, or any combination thereof and can be used as specific biomarkers from exosomes for hematological malignancies.

A biomarker mutation for a blood cancer that can be assessed in an exosome includes, but is not limited to, a mutation of c-kit, PDGFR, or ABL, or any combination of mutations specific for hematological malignancies.

Also provided herein is an isolated exosome comprising one or more blood cancer specific biomarkers, such as listed in FIG. 25 and in FIG. 1 for blood cancer. A composition comprising the isolated exosome is also provided. Accordingly, in some embodiments, the composition comprises a population of exosomes comprising one or more blood cancer specific biomarkers, such as listed in FIG. 25 and in FIG. 1 for blood cancer. The composition can comprise a substantially enriched population of exosomes, wherein the population of exosomes is substantially homogeneous for blood cancer specific exosomes or exosomes comprising one or more blood cancer specific biomarkers, such as listed in FIG. 25 and in FIG. 1 for blood cancer.

One or more blood cancer specific biomarkers, such as listed in FIG. 25 and in FIG. 1 for blood cancer, can also be detected by one or more systems disclosed herein, for characterizing a blood cancer. For example, a detection system can comprise one or more probes to detect one or more blood cancer specific biomarkers, such as listed in FIG. 25 and in FIG. 1 for blood cancer, of one or more exosomes of a biological sample.

The one or more blood cancer specific biomarkers can also be a gene fusion selected from the group consisting of: TTL-ETV6, CDK6-MLL, CDK6-TLX3, ETV6-FLT3, ETV6-RUNX1, ETV6-TTL, MLL-AFF1, MLL-AFF3, MLL-AFF4, MLL-GAS7, TCBA1-ETV6, TCF3-PBX1 or TCF3-TFPT, for acute lymphocytic leukemia (ALL); BCL11B-TLX3, IL2-TNFRFS17, NUP214-ABL1, NUP98-CCDC28A, TAL1-STIL, or ETV6-ABL2, for T-cell acute lymphocytic leukemia (T-ALL); ATIC-ALK, KIAA1618-ALK, MSN-ALK, MYH9-ALK, NPM1-ALK, TGF-ALK or TPM3-ALK, for anaplastic large cell lymphoma (ALCL); BCR-ABL1, BCR-JAK2, ETV6-EVI1, ETV6-MN1 or ETV6-TCBA1, for chronic myelogenous leukemia (CML); CBFB-MYH11, CHIC2-ETV6, ETV6-ABL1, ETV6-ABL2, ETV6-ARNT, ETV6-CDX2, ETV6-HLXB9, ETV6-PER1, MEF2D-DAZAP1, AML-AFF1, MLL-ARHGAP26, MLL-ARHGEF12, MLL-CASC5, MLL-CBL, MLL-CREBBP, MLL-DAB21P, MLL-ELL, MLL-EP300, MLL-EPS15, MLL-FNBP1, MLL-FOXO3A, MLL-GMPS, MLL-GPHN, MLL-MLLT1, MLL-MLLT11, MLL-MLLT3, MLL-MLLT6, MLL-MYO1F, MLL-PICALM, MLL-SEPT2, MLL-SEPT6, MLL-SORBS2, MYST3-SORBS2, MYST-CREBBP, NPM1-MLF1, NUP98-HOXA13, PRDM16-EVI1, RABEP1-PDGFRB, RUNX1-EVI1, RUNX1-MDS1, RUNX1-RPL22, RUNX1-RUNX1T1, RUNX1-SH3D19, RUNX1-USP42, RUNX1-YTHDF2, RUNX1-ZNF687, or TAF15-ZNF-384, for AML; CCND1-FSTL3, for chronic lymphocytic leukemia (CLL); and FLIP1-PDGFRA, FLT3-ETV6, KIAA1509-PDGFRA, PDE4DIP-PDGFRB, NIN-PDGFRB, TP53BP1-PDGFRB, or TPM3-PDGFRB, for hyper eosinophilia/chronic eosinophilia.

The one or more biomarkers for CLL can also include one or more of the following upregulated or overexpressed miRNAs, such as miR-23b, miR-24-1, miR-146, miR-155, miR-195, miR-221, miR-331, miR-29a, miR-195, miR-34a, or miR-29c; one or more of the following downregulated or underexpressed miRs, such as miR-15a, miR-16-1, miR-29 or miR-223, or any combination thereof.

The one or more biomarkers for ALL can also include one or more of the following upregulated or overexpressed miRNAs, such as miR-128b, miR-204, miR-218, miR-331, miR-181b-1, miR-17-92; or any combination thereof.

B-Cell Chronic Lymphocytic Leukemia (B-CLL)

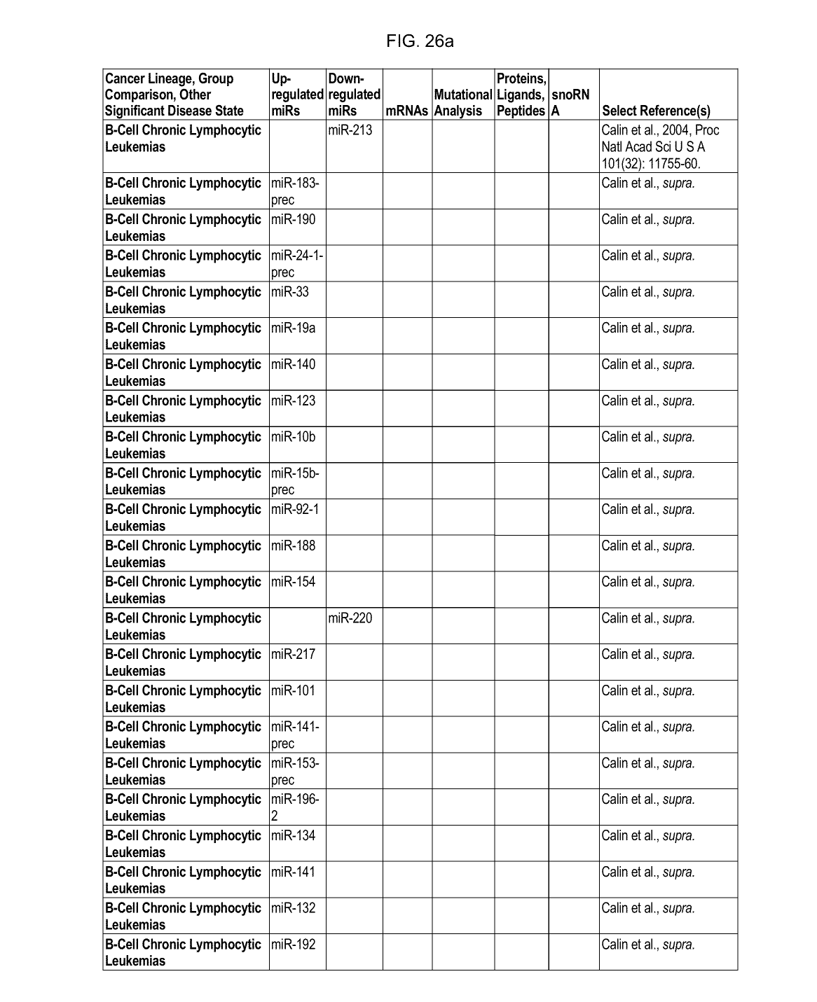

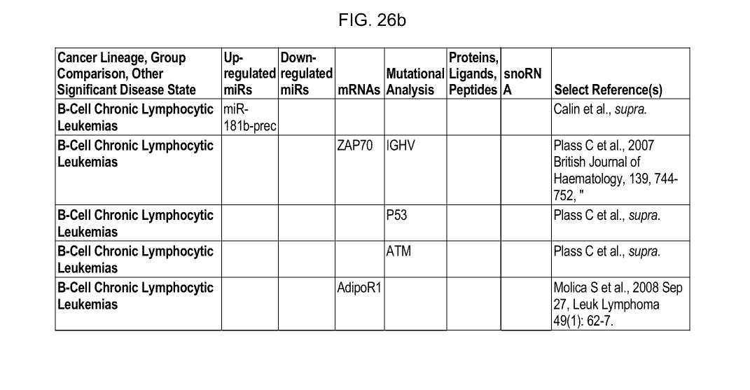

B-CLL specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 26, and can be used to create a B-CLL specific exosome bio-signature. For example, the bio-signature can comprise one or more overexpressed miRs, such as, but not limited to, miR-183-prec, miR-190, miR-24-1-prec, miR-33, miR-19a, miR-140, miR-123, miR-10b, miR-15b-prec, miR-92-1, miR-188, miR-154, miR-217, miR-101, miR-141-prec, miR-153-prec, miR-196-2, miR-134, miR-141, miR-132, miR-192, or miR-181b-prec, or any combination thereof.

The bio-signature can also comprise one or more underexpressed miRs such as, but not limited to, miR-213, miR-220, or any combination thereof. The one or more mRNAs that may be analyzed can include, but are not limited to, ZAP70, AdipoR1, or any combination thereof and can be used as specific biomarkers from exosomes for B-CLL. A biomarker mutation for B-CLL that can be assessed in an exosome includes, but is not limited to, a mutation of IGHV, P53, ATM, or any combination of mutations specific for B-CLL.

Also provided herein is an isolated exosome comprising one or more B-CLL specific biomarkers, such as BCL3-MYC, MYC-BTG1, BCL7A-MYC, BRWD3-ARHGAP20 or BTG1-MYC, or those listed in FIG. 26. A composition comprising the isolated exosome is also provided. Accordingly, in some embodiments, the composition comprises a population of exosomes comprising one or more B-CLL specific biomarkers, such as BCL3-MYC, MYC-BTG1, BCL7A-MYC, BRWD3-ARHGAP20 or BTG1-MYC, or those listed in FIG. 26. The composition can comprise a substantially enriched population of exosomes, wherein the population of exosomes is substantially homogeneous for B-CLL specific exosomes or exosomes comprising one or more B-CLL specific biomarkers, such as BCL3-MYC, MYC-BTG1, BCL7A-MYC, BRWD3-ARHGAP20 or BTG1-MYC, or those listed in FIG. 26.

One or more B-CLL specific biomarkers, such as BCL3-MYC, MYC-BTG1, BCL7A-MYC, BRWD3-ARHGAP20 or BTG1-MYC, or those listed in FIG. 26, can also be detected by one or more systems disclosed herein, for characterizing a B-CLL. For example, a detection system can comprise one or more probes to detect one or more B-CLL specific biomarkers, such as BCL3-MYC, MYC-BTG1, BCL7A-MYC, BRWD3-ARHGAP20 or BTG1-MYC, or those listed in FIG. 26, of one or more exosomes of a biological sample.

B-Cell Lymphoma

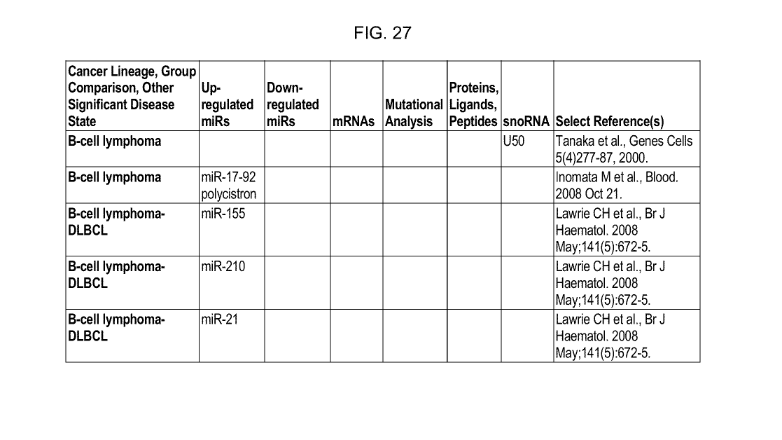

B-cell lymphome specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 27, and can be used to create a B-cell lymphoma specific exosome bio-signature. For example, the bio-signature can comprise one or more overexpressed miRs, such as, but not limited to, miR-17-92 polycistron, miR-155, miR-210, or miR-21, miR-19a, miR-92, miR-142 miR-155, miR-221 miR-17-92, miR-21, miR-191, miR-205, or any combination thereof. Furthermore the snoRNA that can be used as an exosomal biomarker for B-cell lymphoma can include, but is not limited to, U50.

Also provided herein is an isolated exosome comprising one or more B-cell lymphoma specific biomarkers, such as listed in FIG. 27. A composition comprising the isolated exosome is also provided. Accordingly, in some embodiments, the composition comprises a population of exosomes comprising one or more B-cell lymphoma specific biomarkers, such as listed in FIG. 27. The composition can comprise a substantially enriched population of exosomes, wherein the population of exosomes is substantially homogeneous for B-cell lymphoma specific exosomes or exosomes comprising one or more B-cell lymphoma specific biomarkers, such as listed in FIG. 27.

One or more B-cell lymphoma specific biomarkers, such as listed in FIG. 27, can also be detected by one or more systems disclosed herein, for characterizing a B-cell lymphoma. For example, a detection system can comprise one or more probes to detect one or more B-cell lymphoma specific biomarkers, such as listed in FIG. 27, of one or more exosomes of a biological sample.

Diffuse Large B-Cell Lymphoma (DLBCL)

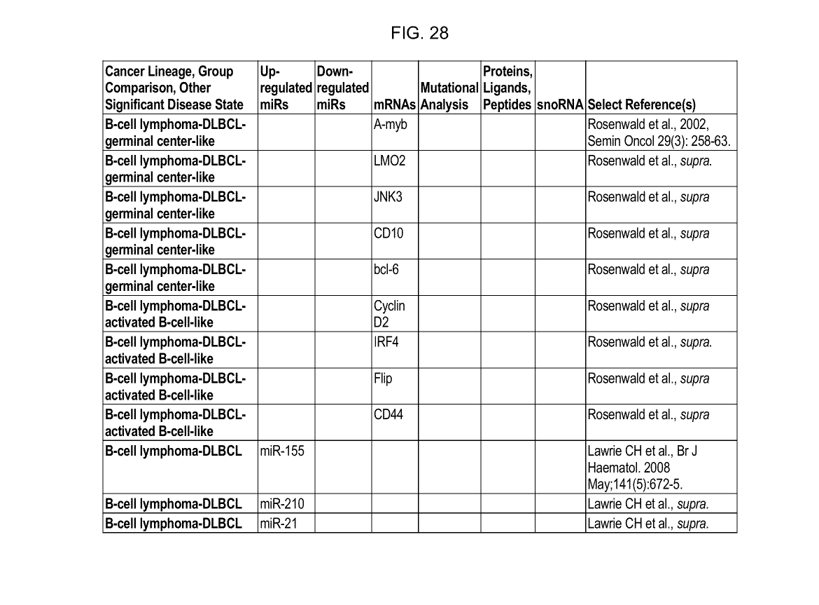

DLBCL specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 28, and can be used to create a DLBCL specific exosome bio-signature. For example, the bio-signature can comprise one or more overexpressed miRs, such as, but not limited to, miR-17-92, miR-155, miR-210, or miR-21, or any combination thereof. The one or more mRNAs that may be analyzed can include, but are not limited to, A-myb, LMO2, JNK3, CD10, bcl-6, Cyclin D2, IRF4, Flip, or CD44, or any combination thereof and can be used as specific biomarkers from exosomes for DLBCL.

Also provided herein is an isolated exosome comprising one or more DLBCL specific biomarkers, such as CITTA-BCL6, CLTC-ALK, IL21R-BCL6, PIM1-BCL6, TFCR-BCL6, IKZF1-BCL6 or SEC31A-ALK, or those listed in FIG. 28. A composition comprising the isolated exosome is also provided. Accordingly, in some embodiments, the composition comprises a population of exosomes comprising one or more DLBCL specific biomarkers, such as CITTA-BCL6, CLTC-ALK, IL21R-BCL6, PIM1-BCL6, TFCR-BCL6, IKZF1-BCL6 or SEC31A-ALK, or those listed in FIG. 28. The composition can comprise a substantially enriched population of exosomes, wherein the population of exosomes is substantially homogeneous for DLBCL specific exosomes or exosomes comprising one or more DLBCL specific biomarkers, such as CITTA-BCL6, CLTC-ALK, IL21R-BCL6, PIM1-BCL6, TFCR-BCL6, IKZF1-BCL6 or SEC31A-ALK, or those listed in FIG. 28.

One or more DLBCL specific biomarkers, such as CITTA-BCL6, CLTC-ALK, IL21R-BCL6, PIM1-BCL6, TFCR-BCL6, IKZF1-BCL6 or SEC31A-ALK, or those listed in FIG. 28, can also be detected by one or more systems disclosed herein, for characterizing a DLBCL. For example, a detection system can comprise one or more probes to detect one or more DLBCL specific biomarkers, such as CITTA-BCL6, CLTC-ALK, IL21R-BCL6, PIM1-BCL6, TFCR-BCL6, IKZF1-BCL6 or SEC31A-ALK, or those listed in FIG. 28, of one or more exosomes of a biological sample.

Burkitt's Lymphoma

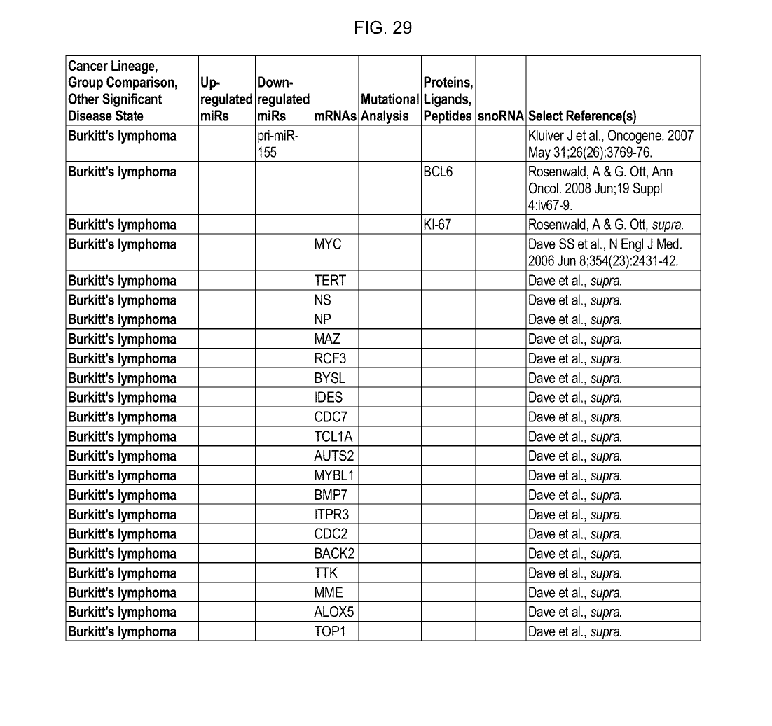

Burkitt's lymphoma specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 29, and can be used to create a Burkitt's lymphoma specific exosome bio-signature. For example, the bio-signature can also comprise one or more underexpressed miRs such as, but not limited to, pri-miR-155, or any combination thereof. The one or more mRNAs that may be analyzed can include, but are not limited to, MYC, TERT, NS, NP, MAZ, RCF3, BYSL, IDE3, CDC7, TCL1A, AUTS2, MYBL1, BMP7, ITPR3, CDC2, BACK2, TTK, MME, ALOX5, or TOP1, or any combination thereof and can be used as specific biomarkers from exosomes for Burkitt's lymphoma. The protein, ligand, or peptide that can be assessed in an exosome can include, but is not limited to, BCL6, KI-67, or any combination thereof.

Also provided herein is an isolated exosome comprising one or more Burkitt's lymphoma specific biomarkers, such as IGH-MYC, LCP1-BCL6, or those listed in FIG. 29. A composition comprising the isolated exosome is also provided. Accordingly, in some embodiments, the composition comprises a population of exosomes comprising one or more Burkitt's lymphoma specific biomarkers, such as IGH-MYC, LCP1-BCL6, or those listed in FIG. 29. The composition can comprise a substantially enriched population of exosomes, wherein the population of exosomes is substantially homogeneous for Burkitt's lymphoma specific exosomes or exosomes comprising one or more Burkitt's lymphoma specific biomarkers, such as IGH-MYC, LCP1-BCL6, or those listed in FIG. 29.

One or more Burkitt's lymphoma specific biomarkers, such as IGH-MYC, LCP1-BCL6, or those listed in FIG. 29, can also be detected by one or more systems disclosed herein, for characterizing a Burkitt's lymphoma. For example, a detection system can comprise one or more probes to detect one or more Burkitt's lymphoma specific biomarkers, such as IGH-MYC, LCP1-BCL6, or those listed in FIG. 29, of one or more exosomes of a biological sample.

Hepatocellular Carcinoma

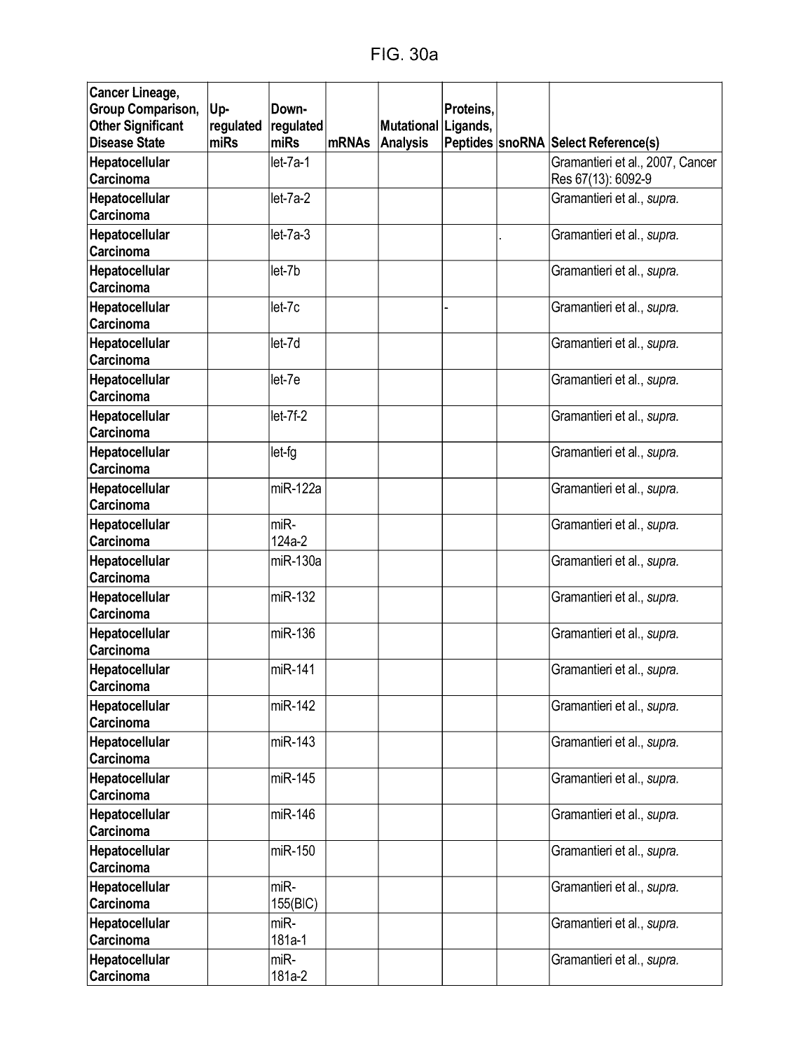

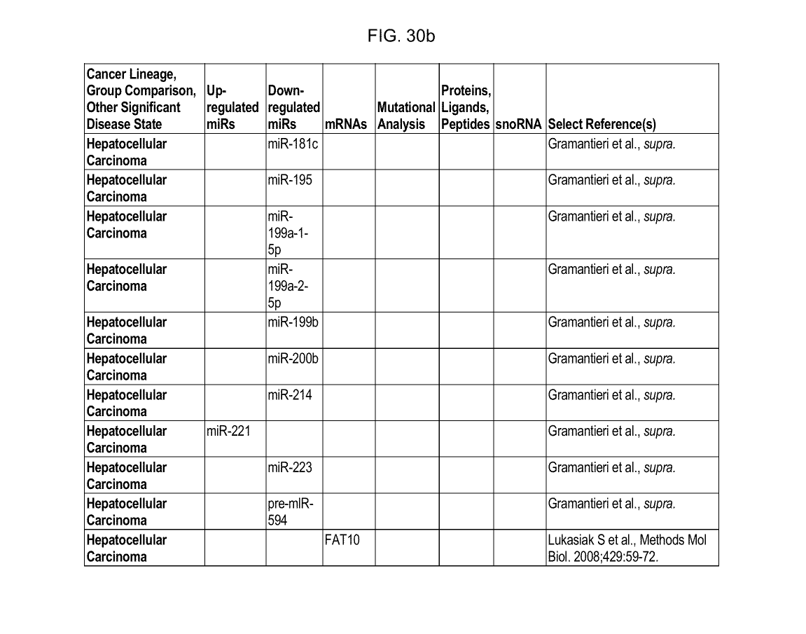

Hepatocellular carcinoma specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 30 and can be used to create a hepatocellular carcinoma specific exosome bio-signature. For example, the bio-signature can comprise one or more overexpressed miRs, such as, but not limited to, miR-221. The bio-signature can also comprise one or more underexpressed miRs such as, but not limited to, let-7a-1, let-7a-2, let-7a-3, let-7b, let-7c, let-7d, let-7e, let-7f-2, let-fg, miR-122a, miR-124a-2, miR-130a, miR-132, miR-136, miR-141, miR-142, miR-143, miR-145, miR-146, miR-150, miR-155(BIC), miR-181a-1, miR-181a-2, miR-181c, miR-195, miR-199a-1-5p, miR-199a-2-5p, miR-199b, miR-200b, miR-214, miR-223, or pre-miR-594, or any combination thereof. The one or more mRNAs that may be analyzed can include, but are not limited to, FAT10.

The one or more biomarkers of a bio-signature can also be used to characterize hepatitis C virus-associated hepatocellular carcinoma. The one or more biomarkers can be a miRNA, such as an overexpressed or underexpressed miRNA. For example, the upregulated or overexpressed miRNA can be miR-122, miR-100, or miR-10a and the downregulated miRNA can be miR-198 or miR-145.

Also provided herein is an isolated exosome comprising one or more hepatocellular carcinoma specific biomarkers, such as listed in FIG. 30 and in FIG. 1 for hepatocellular carcinoma. A composition comprising the isolated exosome is also provided. Accordingly, in some embodiments, the composition comprises a population of exosomes comprising one or more hepatocellular carcinoma specific biomarkers, such as listed in FIG. 30 and in FIG. 1 for hepatocellular carcinoma. The composition can comprise a substantially enriched population of exosomes, wherein the population of exosomes is substantially homogeneous for hepatocellular carcinoma specific exosomes or exosomes comprising one or more hepatocellular carcinoma specific biomarkers, such as listed in FIG. 30 and in FIG. 1 for hepatocellular carcinoma.

One or more hepatocellular carcinoma specific biomarkers, such as listed in FIG. 30 and in FIG. 1 for hepatocellular carcinoma, can also be detected by one or more systems disclosed herein, for characterizing a hepatocellular carcinoma. For example, a detection system can comprise one or more probes to detect one or more hepatocellular carcinoma specific biomarkers, such as listed in FIG. 30 and in FIG. 1 for hepatocellular carcinoma, of one or more exosomes of a biological sample.

Cervical Cancer

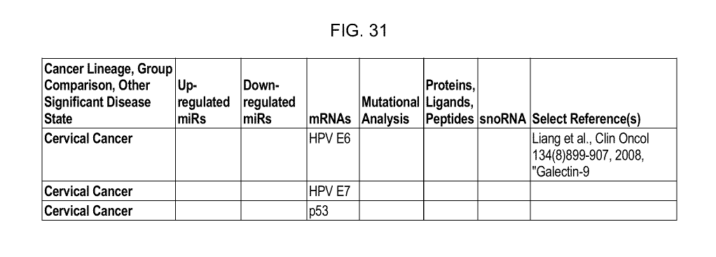

Cervical cancer specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 31, and can be used to create a cervical cancer specific exosome bio-signature. For example, the one or more mRNAs that may be analyzed can include, but are not limited to, HPV E6, HPV E7, or p53, or any combination thereof and can be used as specific biomarkers from exosomes for cervical cancer.

Also provided herein is an isolated exosome comprising one or more cervical cancer specific biomarkers, such as listed in FIG. 31 and in FIG. 1 for cervical cancer. A composition comprising the isolated exosome is also provided. Accordingly, in some embodiments, the composition comprises a population of exosomes comprising one or more cervical cancer specific biomarkers, such as listed in FIG. 31 and in FIG. 1 for cervical cancer. The composition can comprise a substantially enriched population of exosomes, wherein the population of exosomes is substantially homogeneous for cervical cancer specific exosomes or exosomes comprising one or more cervical cancer specific biomarkers, such as listed in FIG. 31 and in FIG. 1 for cervical cancer.

One or more cervical cancer specific biomarkers, such as listed in FIG. 31 and in FIG. 1 for cervical cancer, can also be detected by one or more systems disclosed herein, for characterizing a cervical cancer. For example, a detection system can comprise one or more probes to detect one or more cervical cancer specific biomarkers, such as listed in FIG. 31 and in FIG. 1 for cervical cancer, of one or more exosomes of a biological sample.

Endometrial Cancer

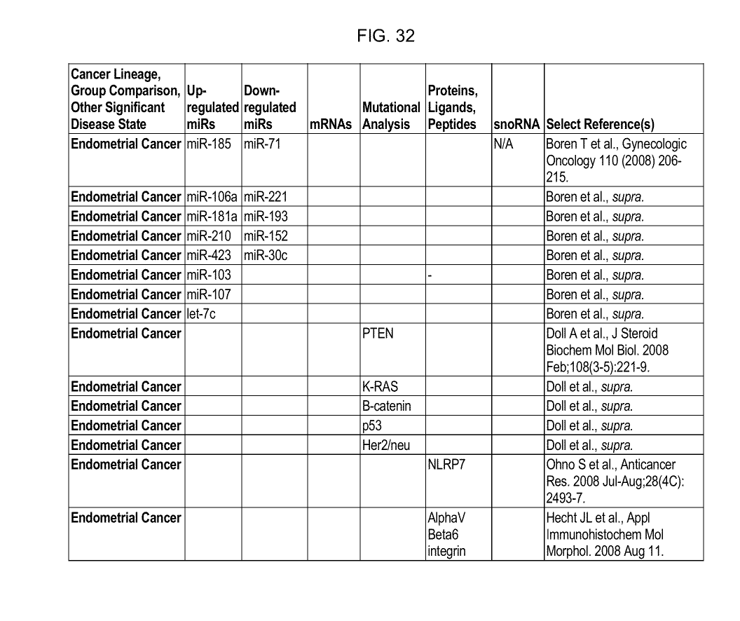

Endometrial cancer specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 32 and can be used to create a endometrial cancer specific exosome bio-signature. For example, the bio-signature can comprise one or more overexpressed miRs, such as, but not limited to, miR-185, miR-106a, miR-181a, miR-210, miR-423, miR-103, miR-107, or let-7c, or any combination thereof. The bio-signature can also comprise one or more underexpressed miRs such as, but not limited to, miR-7i, miR-221, miR-193, miR-152, or miR-30c, or any combination thereof.

A biomarker mutation for endometrial cancer that can be assessed in an exosome includes, but is not limited to, a mutation of PTEN, K-RAS, B-catenin, p53, Her2/neu, or any combination of mutations specific for endometrial cancer. The protein, ligand, or peptide that can be assessed in an exosome can include, but is not limited to, NLRP7, AlphaV Beta6 integrin, or any combination thereof.

Also provided herein is an isolated exosome comprising one or more endometrial cancer specific biomarkers, such as listed in FIG. 32 and in FIG. 1 for endometrial cancer. A composition comprising the isolated exosome is also provided. Accordingly, in some embodiments, the composition comprises a population of exosomes comprising one or more endometrial cancer specific biomarkers, such as listed in FIG. 32 and in FIG. 1 for endometrial cancer. The composition can comprise a substantially enriched population of exosomes, wherein the population of exosomes is substantially homogeneous for endometrial cancer specific exosomes or exosomes comprising one or more endometrial cancer specific biomarkers, such as listed in FIG. 32 and in FIG. 1 for endometrial cancer.

One or more endometrial cancer specific biomarkers, such as listed in FIG. 32 and in FIG. 1 for endometrial cancer, can also be detected by one or more systems disclosed herein, for characterizing a endometrial cancer. For example, a detection system can comprise one or more probes to detect one or more endometrial cancer specific biomarkers, such as listed in FIG. 32 and in FIG. 1 for endometrial cancer, of one or more exosomes of a biological sample.

Head and Neck Cancer

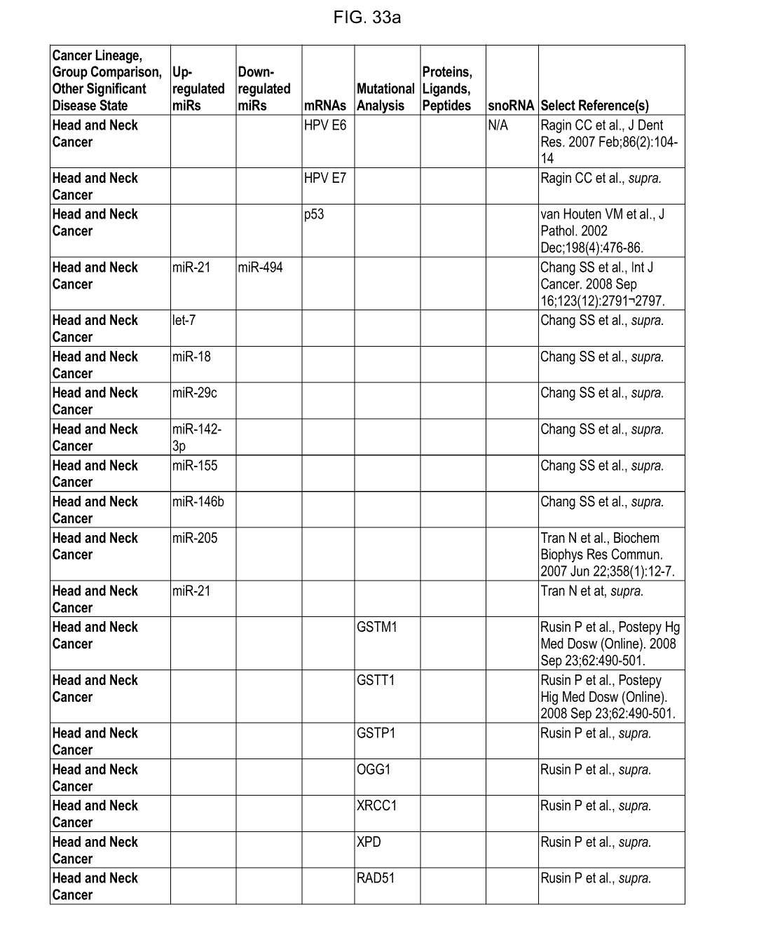

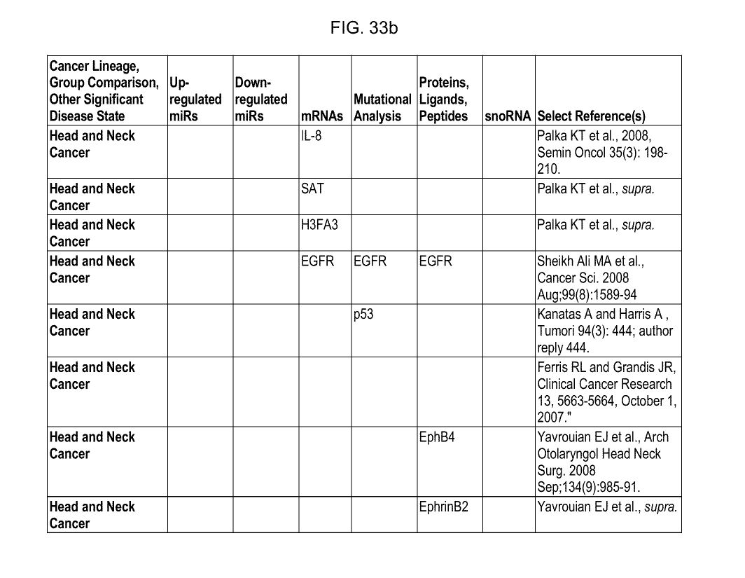

Head and neck cancer specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 33, and can be used to create a head and neck cancer specific exosome bio-signature. For example, the bio-signature can comprise one or more overexpressed miRs, such as, but not limited to, miR-21, let-7, miR-18, miR-29c, miR-142-3p, miR-155, miR-146b, miR-205, or miR-21, or any combination thereof. The bio-signature can also comprise one or more underexpressed miRs such as, but not limited to, miR-494. The one or more mRNAs that may be analyzed include, but are not limited to, HPV E6, HPV E7, p53, IL-8, SAT, H3FA3, or EGFR, or any combination thereof and can be used as specific biomarkers from exosomes for head and neck cancer.

A biomarker mutation for head and neck cancer that can be assessed in an exosome includes, but is not limited to, a mutation of GSTM1, GSTT1, GSTP1, OGG1, XRCC1, XPD, RAD51, EGFR, p53, or any combination of mutations specific for head and neck cancer. The protein, ligand, or peptide that can be assessed in an exosome can include, but is not limited to, EGFR, EphB4, or EphB2, or any combination thereof.

Also provided herein is an isolated exosome comprising one or more head and neck cancer specific biomarkers, such as CHCHD7-PLAG1, CTNNB1-PLAG1, FHIT-HMGA2, HMGA2-NFIB, LIFR-PLAG1, or TCEA1-PLAG1, or those listed in FIG. 33 and in FIG. 1 for head and neck cancer. A composition comprising the isolated exosome is also provided. Accordingly, in some embodiments, the composition comprises a population of exosomes comprising one or more head and neck cancer specific biomarkers, such as CHCHD7-PLAG1, CTNNB1-PLAG1, FHIT-HMGA2, HMGA2-NFIB, LIFR-PLAG1, or TCEA1-PLAG1, or those listed in FIG. 33 and in FIG. 1 for head and neck cancer. The composition can comprise a substantially enriched population of exosomes, wherein the population of exosomes is substantially homogeneous for head and neck cancer specific exosomes or exosomes comprising one or more head and neck cancer specific biomarkers, such as CHCHD7-PLAG1, CTNNB1-PLAG1, FHIT-HMGA2, HMGA2-NFIB, LIFR-PLAG1, or TCEA1-PLAG1, or those listed in FIG. 33 and in FIG. 1 for head and neck cancer.

One or more head and neck cancer specific biomarkers, such as listed in FIG. 33 and in FIG. 1 for head and neck cancer, can also be detected by one or more systems disclosed herein, for characterizing a head and neck cancer. For example, a detection system can comprise one or more probes to detect one or more head and neck cancer specific biomarkers, such as CHCHD7-PLAG1, CTNNB1-PLAG1, FHIT-HMGA2, HMGA2-NFIB, LIFR-PLAG1, or TCEA1-PLAG1, or those listed in FIG. 33 and in FIG. 1 for head and neck cancer, of one or more exosomes of a biological sample.

Inflammatory Bowel Disease (IBD)

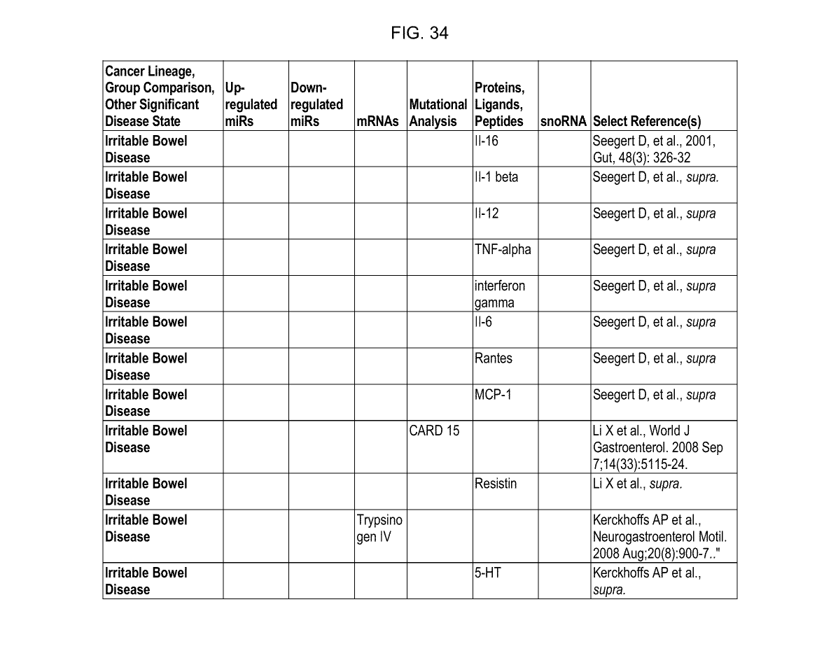

IBD specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 34, and can be used to create a IBD specific exosome bio-signature. The one or more mRNAs that may be analyzed can include, but are not limited to, Trypsinogen IV, SERT, or any combination thereof and can be used as specific biomarkers from exosomes for IBD.

A biomarker mutation for IBD that can be assessed in an exosome can include, but is not limited to, a mutation of CARD15 or any combination of mutations specific for IBD. The protein, ligand, or peptide that can be assessed in an exosome can include, but is not limited to, II-16, II-1beta, II-12, TNF-alpha, interferon gamma, II-6, Rantes, MCP-1, Resistin, or 5-HT, or any combination thereof.

Also provided herein is an isolated exosome comprising one or more IBD specific biomarkers, such as listed in FIG. 34 and in FIG. 1 for IBD. A composition comprising the isolated exosome is also provided. Accordingly, in some embodiments, the composition comprises a population of exosomes comprising one or more IBD specific biomarkers, such as listed in FIG. 34 and in FIG. 1 for IBD. The composition can comprise a substantially enriched population of exosomes, wherein the population of exosomes is substantially homogeneous for IBD specific exosomes or exosomes comprising one or more IBD specific biomarkers, such as listed in FIG. 34 and in FIG. 1 for IBD.

One or more IBD specific biomarkers, such as listed in FIG. 34 and in FIG. 1 for IBD, can also be detected by one or more systems disclosed herein, for characterizing a IBD. For example, a detection system can comprise one or more probes to detect one or more IBD specific biomarkers, such as listed in FIG. 34 and in FIG. 1 for IBD, of one or more exosomes of a biological sample.

Diabetes

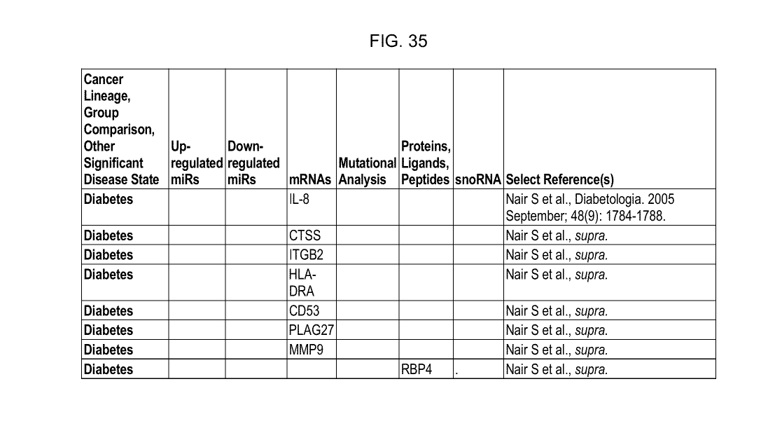

Diabetes specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 35, and can be used to create a diabetes specific exosome bio-signature. For example, the one or more mRNAs that may be analyzed can include, but are not limited to, II-8, CTSS, ITGB2, HLA-DRA, CD53, PLAG27, or MMP9, or any combination thereof and can be used as specific biomarkers from exosomes for diabetes. The protein, ligand, or peptide that can be assessed in an exosome can include, but is not limited to, RBP4.

Also provided herein is an isolated exosome comprising one or more diabetes specific biomarkers, such as listed in FIG. 35 and in FIG. 1 for diabetes. A composition comprising the isolated exosome is also provided. Accordingly, in some embodiments, the composition comprises a population of exosomes comprising one or more diabetes specific biomarkers, such as listed in FIG. 35 and in FIG. 1 for diabetes. The composition can comprise a substantially enriched population of exosomes, wherein the population of exosomes is substantially homogeneous for diabetes specific exosomes or exosomes comprising one or more diabetes specific biomarkers, such as listed in FIG. 35 and in FIG. 1 for diabetes.

One or more diabetes specific biomarkers, such as listed in FIG. 35 and in FIG. 1 for diabetes, can also be detected by one or more systems disclosed herein, for characterizing a diabetes. For example, a detection system can comprise one or more probes to detect one or more diabetes specific biomarkers, such as listed in FIG. 35 and in FIG. 1 for diabetes, of one or more exosomes of a biological sample.

Barrett's Esophagus

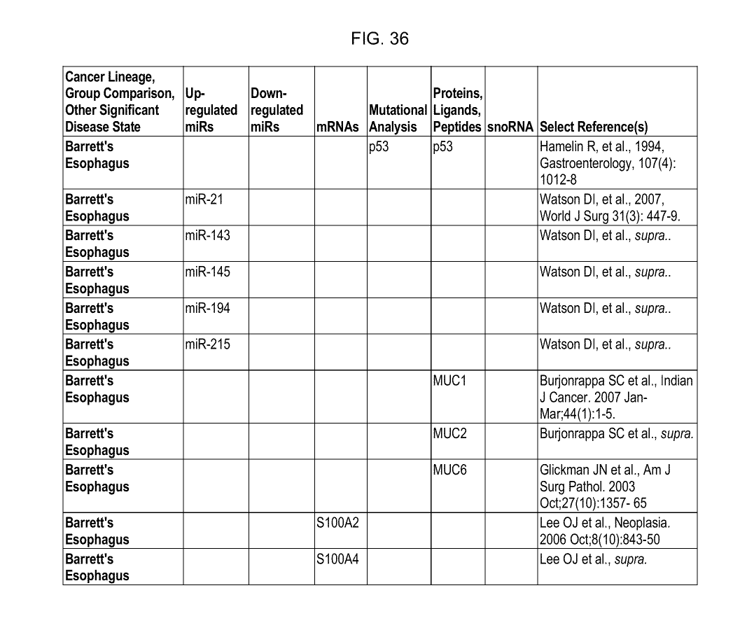

Barrett's Esophagus specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 36, and can be used to create a Barrett's Esophagus specific exosome bio-signature. For example, the bio-signature can comprise one or more overexpressed miRs, such as, but not limited to, miR-21, miR-143, miR-145, miR-194, or miR-215, or any combination thereof. The one or more mRNAs that may be analyzed include, but are not limited to, S100A2, S100A4, or any combination thereof and can be used as specific biomarkers from exosomes for Barrett's Esophagus.

A biomarker mutation for Barrett's Esophagus that can be assessed in an exosome includes, but is not limited to, a mutation of p53 or any combination of mutations specific for Barrett's Esophagus. The protein, ligand, or peptide that can be assessed in an exosome can include, but is not limited to, p53, MUC1, MUC2, or any combination thereof.

Also provided herein is an isolated exosome comprising one or more Barrett's Esophagus specific biomarkers, such as listed in FIG. 36 and in FIG. 1 for Barrett's Esophagus. A composition comprising the isolated exosome is also provided. Accordingly, in some embodiments, the composition comprises a population of exosomes comprising one or more Barrett's Esophagus specific biomarkers, such as listed in FIG. 36 and in FIG. 1 for Barrett's Esophagus. The composition can comprise a substantially enriched population of exosomes, wherein the population of exosomes is substantially homogeneous for Barrett's Esophagus specific exosomes or exosomes comprising one or more Barrett's Esophagus specific biomarkers, such as listed in FIG. 36 and in FIG. 1 for Barrett's Esophagus.

One or more Barrett's Esophagus specific biomarkers, such as listed in FIG. 36 and in FIG. 1 for Barrett's Esophagus, can also be detected by one or more systems disclosed herein, for characterizing a Barrett's Esophagus. For example, a detection system can comprise one or more probes to detect one or more Barrett's Esophagus specific biomarkers, such as listed in FIG. 36 and in FIG. 1 for Barrett's Esophagus, of one or more exosomes of a biological sample.

Fibromyalgia

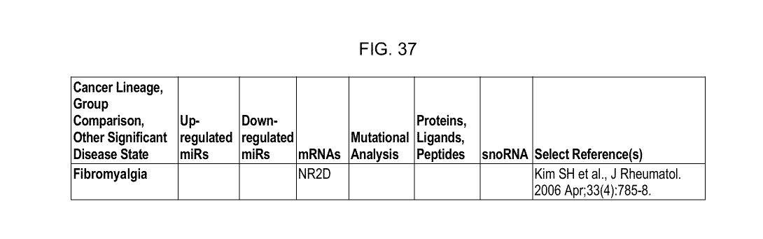

Fibromyalgia specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 37, and can be used to create a fibromyalgia specific exosome bio-signature. The one or more mRNAs that may be analyzed can include, but are not limited to, NR2D which can be used as a specific biomarker from exosomes for fibromyalgia.

Also provided herein is an isolated exosome comprising one or more fibromyalgia specific biomarkers, such as listed in FIG. 37 and in FIG. 1 for fibromyalgia. A composition comprising the isolated exosome is also provided. Accordingly, in some embodiments, the composition comprises a population of exosomes comprising one or more fibromyalgia specific biomarkers, such as listed in FIG. 37 and in FIG. 1 for fibromyalgia. The composition can comprise a substantially enriched population of exosomes, wherein the population of exosomes is substantially homogeneous for fibromyalgia specific exosomes or exosomes comprising one or more fibromyalgia specific biomarkers, such as listed in FIG. 37 and in FIG. 1 for fibromyalgia.

One or more fibromyalgia specific biomarkers, such as listed in FIG. 37 and in FIG. 1 for fibromyalgia, can also be detected by one or more systems disclosed herein, for characterizing a fibromyalgia. For example, a detection system can comprise one or more probes to detect one or more fibromyalgia specific biomarkers, such as listed in FIG. 37 and in FIG. 1 for fibromyalgia, of one or more exosomes of a biological sample.

Stroke

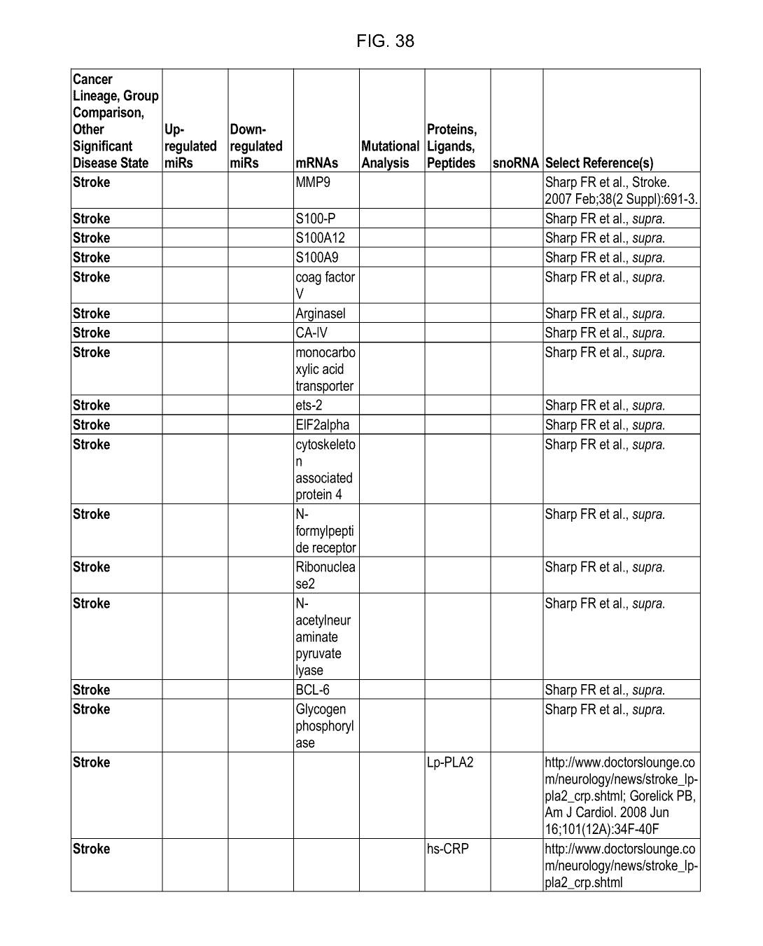

Stroke specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 38, and can be used to create a stroke specific exosome bio-signature. For example, the one or more mRNAs that may be analyzed can include, but are not limited to, MMP9, S100-P, S100A12, S100A9, coag factor V, Arginasel, CA-IV, monocarboxylic acid transporter, ets-2, EIF2alpha, cytoskeleton associated protein 4, N-formylpeptide receptor, Ribonuclease2, N-acetylneuraminate pyruvate lyase, BCL-6, or Glycogen phosphorylase, or any combination thereof and can be used as specific biomarkers from exosomes for stroke.

Also provided herein is an isolated exosome comprising one or more stroke specific biomarkers, such as listed in FIG. 38 and in FIG. 1 for stroke. A composition comprising the isolated exosome is also provided. Accordingly, in some embodiments, the composition comprises a population of exosomes comprising one or more stroke specific biomarkers, such as listed in FIG. 38 and in FIG. 1 for stroke. The composition can comprise a substantially enriched population of exosomes, wherein the population of exosomes is substantially homogeneous for stroke specific exosomes or exosomes comprising one or more stroke specific biomarkers, such as listed in FIG. 38 and in FIG. 1 for stroke.

One or more stroke specific biomarkers, such as listed in FIG. 38 and in FIG. 1 for stroke, can also be detected by one or more systems disclosed herein, for characterizing a stroke. For example, a detection system can comprise one or more probes to detect one or more stroke specific biomarkers, such as listed in FIG. 38 and in FIG. 1 for stroke, of one or more exosomes of a biological sample.

Multiple Sclerosis (MS)

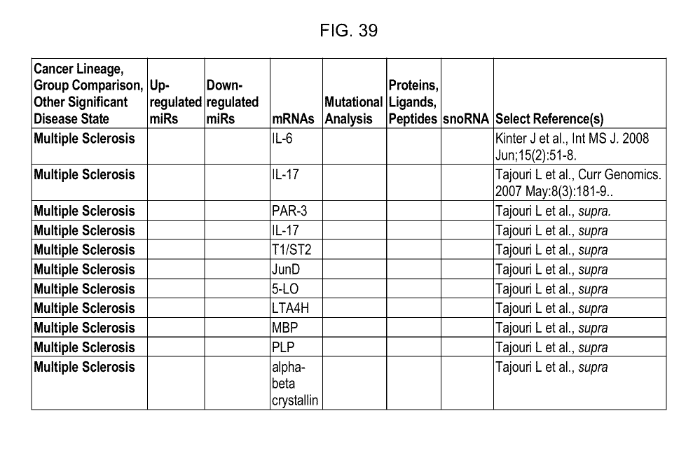

MS specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 39, and can be used to create a MS specific exosome bio-signature. For example, the one or more mRNAs that may be analyzed can include, but are not limited to, IL-6, IL-17, PAR-3, IL-17, T1/ST2, JunD, 5-LO, LTA4H, MBP, PLP, or alpha-beta crystallin, or any combination thereof and can be used as specific biomarkers from exosomes for MS.

Also provided herein is an isolated exosome comprising one or more MS specific biomarkers, such as listed in FIG. 39 and in FIG. 1 for MS. A composition comprising the isolated exosome is also provided. Accordingly, in some embodiments, the composition comprises a population of exosomes comprising one or more MS specific biomarkers, such as listed in FIG. 39 and in FIG. 1 for MS. The composition can comprise a substantially enriched population of exosomes, wherein the population of exosomes is substantially homogeneous for MS specific exosomes or exosomes comprising one or more MS specific biomarkers, such as listed in FIG. 39 and in FIG. 1 for MS.

One or more MS specific biomarkers, such as listed in FIG. 39 and in FIG. 1 for MS, can also be detected by one or more systems disclosed herein, for characterizing a MS. For example, a detection system can comprise one or more probes to detect one or more MS specific biomarkers, such as listed in FIG. 39 and in FIG. 1 for MS, of one or more exosomes of a biological sample.

Parkinson's Disease

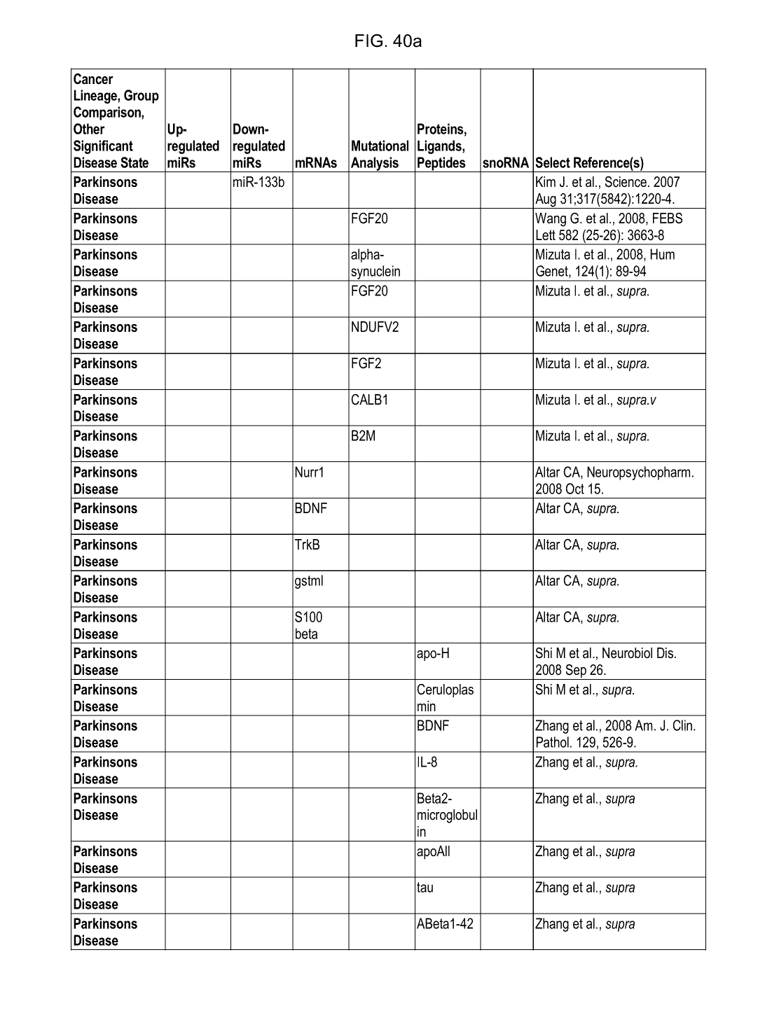

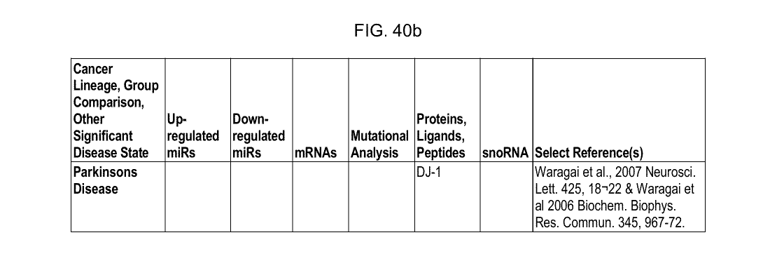

Parkinson's disease specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 40, and can be used to create a Parkinson's disease specific exosome bio-signature. For example, the bio-signature can include, but is not limited to, one or more underexpressed miRs such as miR-133b. The one or more mRNAs that may be analyzed can include, but are not limited to Nurrl, BDNF, TrkB, gstm1, or S100 beta, or any combination thereof and can be used as specific biomarkers from exosomes for Parkinson's disease.

A biomarker mutation for Parkinson's disease that can be assessed in an exosome includes, but is not limited to, a mutation of FGF20, alpha-synuclein, FGF20, NDUFV2, FGF2, CALB1, B2M, or any combination of mutations specific for Parkinson's disease. The protein, ligand, or peptide that can be assessed in an exosome can include, but is not limited to, apo-H, Ceruloplasmin, BDNF, IL-8, Beta2-microglobulin, apoAII, tau, ABeta1-42, DJ-1, or any combination thereof.

Also provided herein is an isolated exosome comprising one or more Parkinson's disease specific biomarkers, such as listed in FIG. 40 and in FIG. 1 for Parkinson's disease A composition comprising the isolated exosome is also provided. Accordingly, in some embodiments, the composition comprises a population of exosomes comprising one or more Parkinson's disease specific biomarkers, such as listed in FIG. 40 and in FIG. 1 for Parkinson's disease. The composition can comprise a substantially enriched population of exosomes, wherein the population of exosomes is substantially homogeneous for Parkinson's disease specific exosomes or exosomes comprising one or more Parkinson's disease specific biomarkers, such as listed in FIG. 40 and in FIG. 1 for Parkinson's disease.

One or more Parkinson's disease specific biomarkers, such as listed in FIG. 40 and in FIG. 1 for Parkinson's disease, can also be detected by one or more systems disclosed herein, for characterizing a Parkinson's disease. For example, a detection system can comprise one or more probes to detect one or more Parkinson's disease specific biomarkers, such as listed in FIG. 40 and in FIG. 1 for Parkinson's disease, of one or more exosomes of a biological sample.

Rheumatic Disease

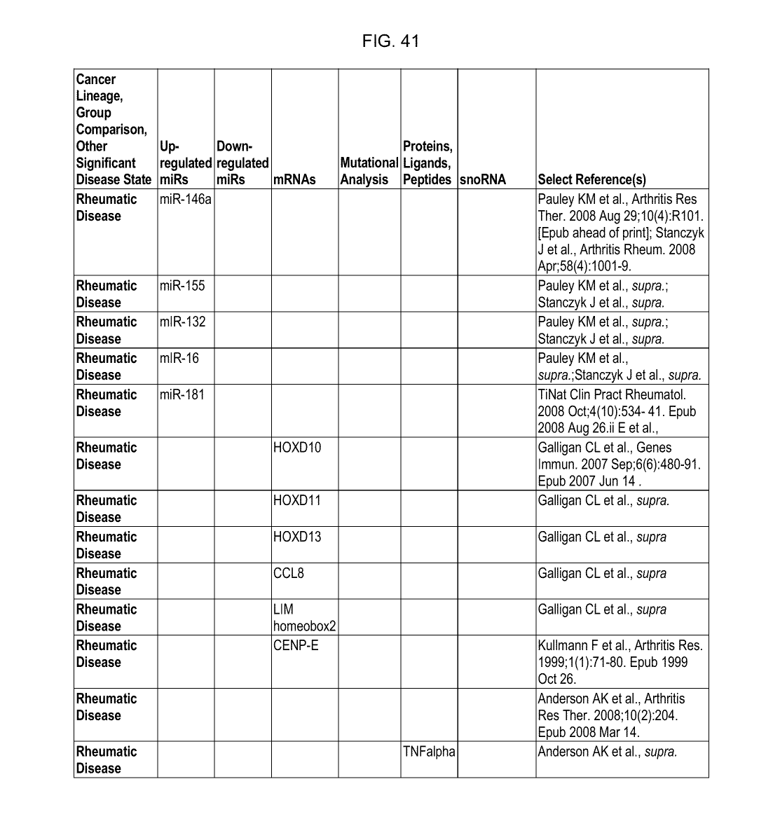

Rheumatic disease specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 41, and can be used to create a rheumatic disease specific exosome bio-signature. For example, the bio-signature can also comprise one or more underexpressed miRs such as, but not limited to, miR-146a, miR-155, miR-132, miR-16, or miR-181, or any combination thereof. The one or more mRNAs that may be analyzed can include, but are not limited to, HOXD10, HOXD11, HOXD13, CCL8, LIM homeobox2, or CENP-E, or any combination thereof and can be used as specific biomarkers from exosomes for rheumatic disease. The protein, ligand, or peptide that can be assessed in an exosome can include, but is not limited to, TNFα.

Also provided herein is an isolated exosome comprising one or more rheumatic disease specific biomarkers, such as listed in FIG. 41 and in FIG. 1 for rheumatic disease. A composition comprising the isolated exosome is also provided. Accordingly, in some embodiments, the composition comprises a population of exosomes comprising one or more rheumatic disease specific biomarkers, such as listed in FIG. 41 and in FIG. 1 for rheumatic disease. The composition can comprise a substantially enriched population of exosomes, wherein the population of exosomes is substantially homogeneous for rheumatic disease specific exosomes or exosomes comprising one or more rheumatic disease specific biomarkers, such as listed in FIG. 41 and in FIG. 1 for rheumatic disease.

One or more rheumatic disease specific biomarkers, such as listed in FIG. 41 and in FIG. 1 for rheumatic disease, can also be detected by one or more systems disclosed herein, for characterizing a rheumatic disease. For example, a detection system can comprise one or more probes to detect one or more rheumatic disease specific biomarkers, such as listed in FIG. 41 and in FIG. 1 for rheumatic disease, of one or more exosomes of a biological sample.

Alzheimer's Disease

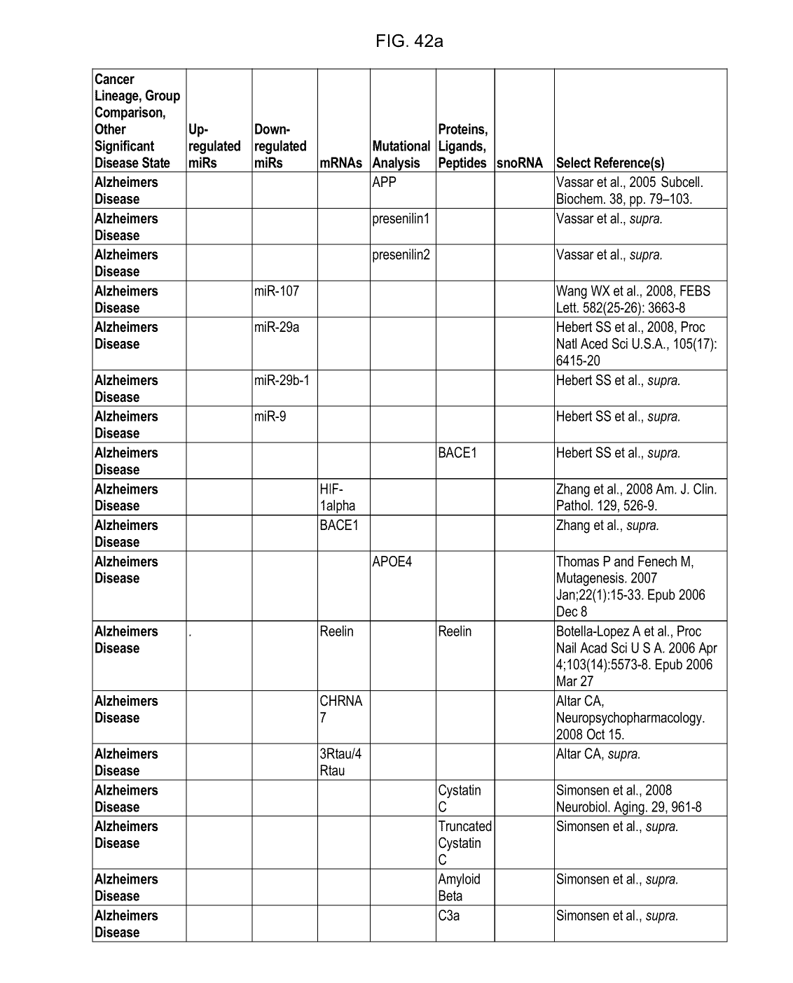

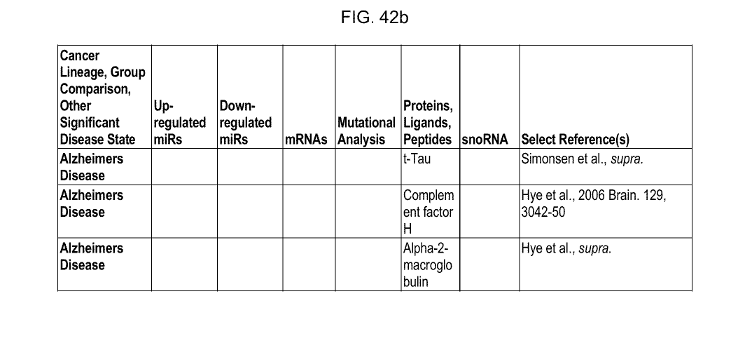

Alzheimer's disease specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 42, and can be used to create a Alzheimers disease specific exosome bio-signature. For example, the bio-signature can also comprise one or more underexpressed miRs such as miR-107, miR-29a, miR-29b-1, or miR-9, or any combination thereof. The bio-signature can also comprise one or more overexpressed miRs such as miR-128 or any combination thereof.

The one or more mRNAs that may be analyzed can include, but are not limited to, HIF-1α, BACE1, Reelin, CHRNA7, or 3Rtau/4Rtau, or any combination thereof and can be used as specific biomarkers from exosomes for Alzheimer's disease.

A biomarker mutation for Alzheimer's disease that can be assessed in an exosome includes, but is not limited to, a mutation of APP, presenilin1, presenilin2, APOE4, or any combination of mutations specific for Alzheimer's disease. The protein, ligand, or peptide that can be assessed in an exosome can include, but is not limited to, BACE1, Reelin, Cystatin C, Truncated Cystatin C, Amyloid Beta, C3a, t-Tau, Complement factor H, or alpha-2-macroglobulin, or any combination thereof.

Also provided herein is an isolated exosome comprising one or more Alzheimer's disease specific biomarkers, such as listed in FIG. 42 and in FIG. 1 for Alzheimer's disease. A composition comprising the isolated exosome is also provided. Accordingly, in some embodiments, the composition comprises a population of exosomes comprising one or more Alzheimer's disease specific biomarkers, such as listed in FIG. 42 and in FIG. 1 for Alzheimer's disease. The composition can comprise a substantially enriched population of exosomes, wherein the population of exosomes is substantially homogeneous for Alzheimer's disease specific exosomes or exosomes comprising one or more Alzheimer's disease specific biomarkers, such as listed in FIG. 42 and in FIG. 1 for Alzheimer's disease.

One or more Alzheimer's disease specific biomarkers, such as listed in FIG. 42 and in FIG. 1 for Alzheimer's disease, can also be detected by one or more systems disclosed herein, for characterizing a Alzheimer's disease. For example, a detection system can comprise one or more probes to detect one or more Alzheimer's disease specific biomarkers, such as listed in FIG. 42 and in FIG. 1 for Alzheimer's disease, of one or more exosomes of a biological sample.

Prion Disease

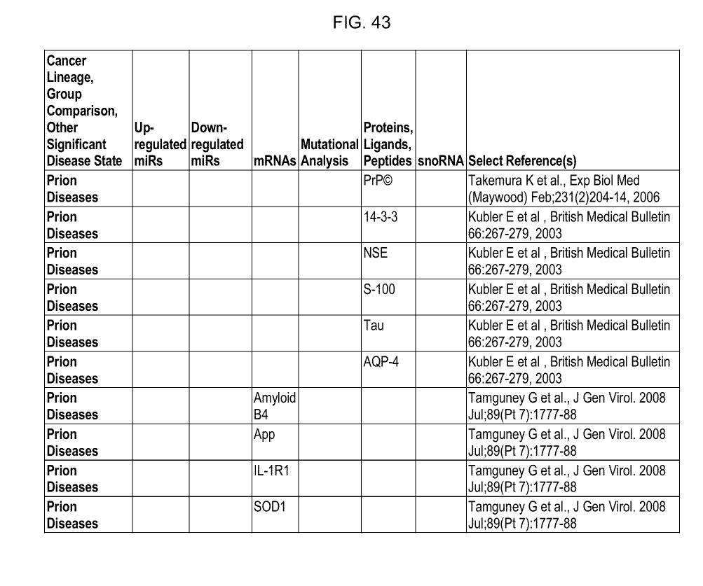

Prion specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 43, and can be used to create a prion specific exosome bio-signature. For example, the one or more mRNAs that may be analyzed can include, but are not limited to, Amyloid B4, App, IL-1R1, or SOD1, or any combination thereof and can be used as specific biomarkers from exosomes for a prion. The protein, ligand, or peptide that can be assessed in an exosome can include, but is not limited to, PrP(c), 14-3-3, NSE, S-100, Tau, AQP-4, or any combination thereof.

Also provided herein is an isolated exosome comprising one or more prion disease specific biomarkers, such as listed in FIG. 43 and in FIG. 1 for prion disease. A composition comprising the isolated exosome is also provided. Accordingly, in some embodiments, the composition comprises a population of exosomes comprising one or more prion disease specific biomarkers, such as listed in FIG. 43 and in FIG. 1 for prion disease. The composition can comprise a substantially enriched population of exosomes, wherein the population of exosomes is substantially homogeneous for prion disease specific exosomes or exosomes comprising one or more prion disease specific biomarkers, such as listed in FIG. 43 and in FIG. 1 for prion disease.

One or more prion disease specific biomarkers, such as listed in FIG. 43 and in FIG. 1 for prion disease, can also be detected by one or more systems disclosed herein, for characterizing a prion disease. For example, a detection system can comprise one or more probes to detect one or more prion disease specific biomarkers, such as listed in FIG. 43 and in FIG. 1 for prion disease, of one or more exosomes of a biological sample.

Sepsis

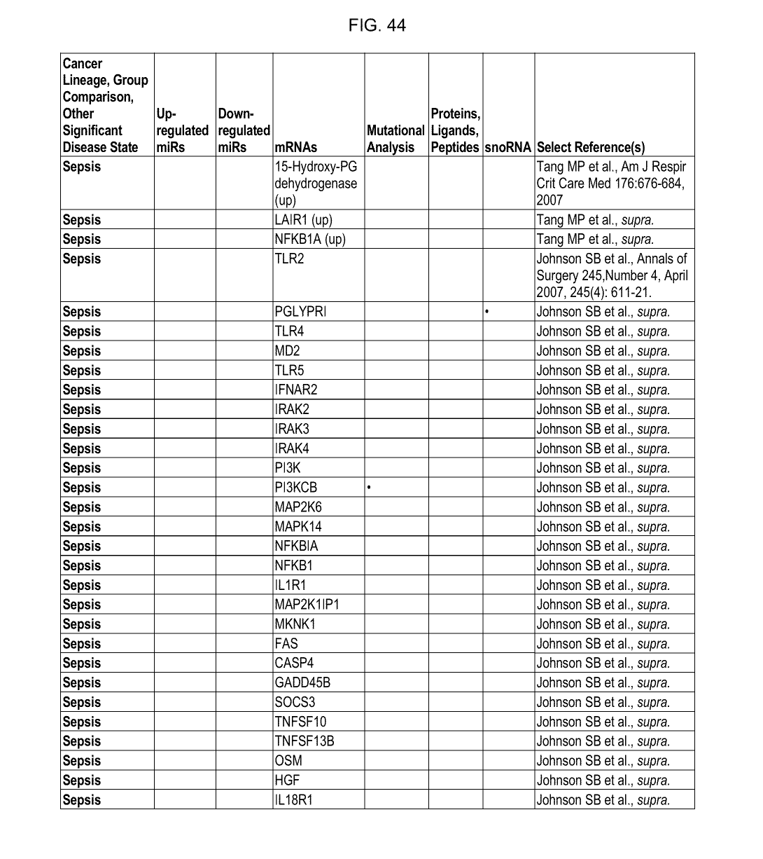

Sepsis specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 44, and can be used to create a sepsis specific exosome bio-signature. For example, the one or more mRNAs that may be analyzed can include, but are not limited to, 15-Hydroxy-PG dehydrogenase (up), LAIR1 (up), NFKB1A (up), TLR2, PGLYPR1, TLR4, MD2, TLR5, IFNAR2, IRAK2, IRAK3, IRAK4, PI3K, PI3KCB, MAP2K6, MAPK14, NFKB1A, NFKB1, IL1R1, MAP2K1IP1, MKNK1, FAS, CASP4, GADD45B, SOCS3, TNFSF10, TNFSF13B, OSM, HGF, or IL18R1, or any combination thereof and can be used as specific biomarkers from exosomes for sepsis.

Also provided herein is an isolated exosome comprising one or more sepsis specific biomarkers, such as listed in FIG. 44. A composition comprising the isolated exosome is also provided. Accordingly, in some embodiments, the composition comprises a population of exosomes comprising one or more sepsis specific biomarkers, such as listed in FIG. 44. The composition can comprise a substantially enriched population of exosomes, wherein the population of exosomes is substantially homogeneous for sepsis specific exosomes or exosomes comprising one or more sepsis specific biomarkers, such as listed in FIG. 44.

One or more sepsis specific biomarkers, such as listed in FIG. 44, can also be detected by one or more systems disclosed herein, for characterizing a sepsis. For example, a detection system can comprise one or more probes to detect one or more sepsis specific biomarkers, such as listed in FIG. 44, of one or more exosomes of a biological sample.

Chronic Neuropathic Pain

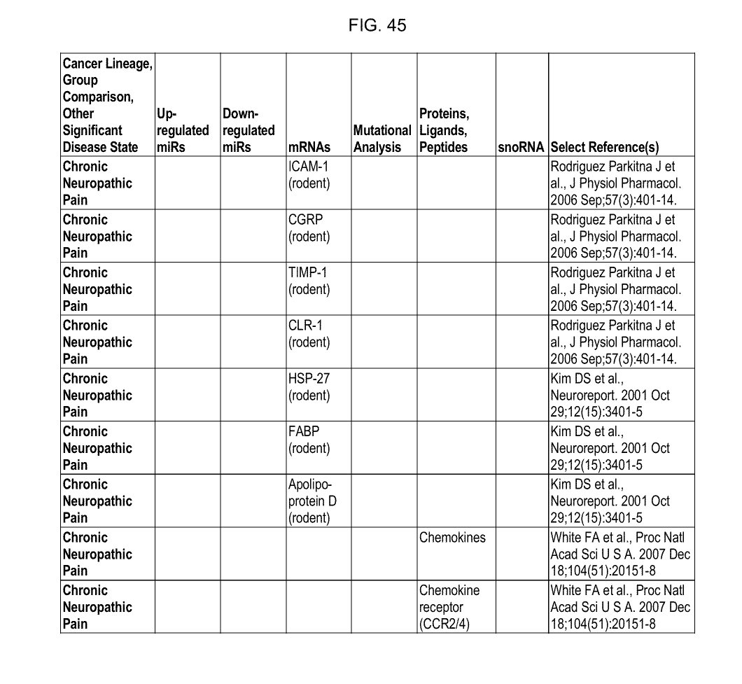

Chronic neuropathic pain (CNP) specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 45, and can be used to create a CNP specific exosome bio-signature. For example, the one or more mRNAs that may be analyzed can include, but are not limited to, ICAM-1 (rodent), CGRP (rodent), TIMP-1 (rodent), CLR-1 (rodent), HSP-27 (rodent), FABP (rodent), or apolipoprotein D (rodent), or any combination thereof and can be used as specific biomarkers from exosomes for CNP. The protein, ligand, or peptide that can be assessed in an exosome can include, but is not limited to, chemokines, chemokine receptors (CCR2/4), or any combination thereof.

Also provided herein is an isolated exosome comprising one or more chronic neuropathic pain specific biomarkers, such as listed in FIG. 45 and in FIG. 1 for chronic neuropathic pain. A composition comprising the isolated exosome is also provided. Accordingly, in some embodiments, the composition comprises a population of exosomes comprising one or more chronic neuropathic pain specific biomarkers, such as listed in FIG. 45 and in FIG. 1 for chronic neuropathic pain. The composition can comprise a substantially enriched population of exosomes, wherein the population of exosomes is substantially homogeneous for chronic neuropathic pain specific exosomes or exosomes comprising one or more chronic neuropathic pain specific biomarkers, such as listed in FIG. 45 and in FIG. 1 for chronic neuropathic pain.

One or more chronic neuropathic pain specific biomarkers, such as listed in FIG. 45 and in FIG. 1 for chronic neuropathic pain, can also be detected by one or more systems disclosed herein, for characterizing a chronic neuropathic pain. For example, a detection system can comprise one or more probes to detect one or more chronic neuropathic pain specific biomarkers, such as listed in FIG. 45 and in FIG. 1 for chronic neuropathic pain, of one or more exosomes of a biological sample.

Peripheral Neuropathic Pain

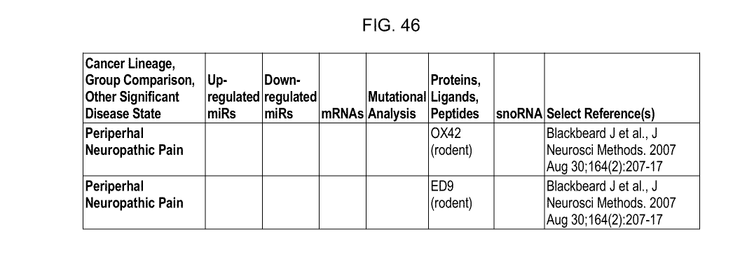

Peripheral neuropathic pain (PNP) specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 46, and can be used to create a PNP specific exosome bio-signature. For example, the protein, ligand, or peptide that can be assessed in an exosome can include, but is not limited to, OX42, ED9, or any combination thereof.

Also provided herein is an isolated exosome comprising one or more peripheral neuropathic pain specific biomarkers, such as listed in FIG. 46 and in FIG. 1 for peripheral neuropathic pain. A composition comprising the isolated exosome is also provided. Accordingly, in some embodiments, the composition comprises a population of exosomes comprising one or more peripheral neuropathic pain specific biomarkers, such as listed in FIG. 46 and in FIG. 1 for peripheral neuropathic pain. The composition can comprise a substantially enriched population of exosomes, wherein the population of exosomes is substantially homogeneous for peripheral neuropathic pain specific exosomes or exosomes comprising one or more peripheral neuropathic pain specific biomarkers, such as listed in FIG. 46 and in FIG. 1 for peripheral neuropathic pain.

One or more peripheral neuropathic pain specific biomarkers, such as listed in FIG. 46 and in FIG. 1 for peripheral neuropathic pain, can also be detected by one or more systems disclosed herein, for characterizing a peripheral neuropathic pain. For example, a detection system can comprise one or more probes to detect one or more peripheral neuropathic pain specific biomarkers, such as listed in FIG. 46 and in FIG. 1 for peripheral neuropathic pain, of one or more exosomes of a biological sample.

Schizophrenia

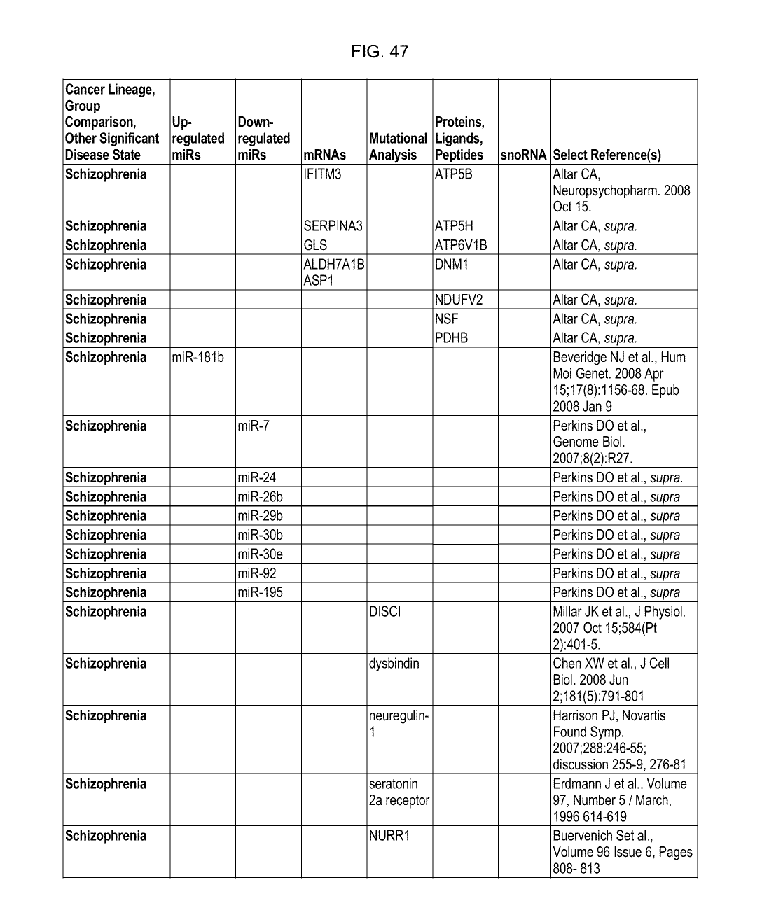

Schizophrenia specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 47, and can be used to create a schizophrenia specific exosome bio-signature. For example, the bio-signature can comprise one or more overexpressed miRs, such as, but not limited to, miR-181b. The bio-signature can also comprise one or more underexpressed miRs such as, but not limited to, miR-7, miR-24, miR-26b, miR-29b, miR-30b, miR-30e, miR-92, or miR-195, or any combination thereof.

The one or more mRNAs that may be analyzed can include, but are not limited to, IFITM3, SERPINA3, GLS, or ALDH7A1BASP1, or any combination thereof and can be used as specific biomarkers from exosomes for schizophrenia. A biomarker mutation for schizophrenia that can be assessed in an exosome includes, but is not limited to, a mutation of to DISC 1, dysbindin, neuregulin-1, seratonin 2a receptor, NURR1, or any combination of mutations specific for schizophrenia.

The protein, ligand, or peptide that can be assessed in an exosome can include, but is not limited to, ATP5B, ATP5H, ATP6V1B, DNM1, NDUFV2, NSF, PDHB, or any combination thereof.

Also provided herein is an isolated exosome comprising one or more schizophrenia specific biomarkers, such as listed in FIG. 47 and in FIG. 1 for schizophrenia. A composition comprising the isolated exosome is also provided. Accordingly, in some embodiments, the composition comprises a population of exosomes comprising one or more schizophrenia specific biomarkers, such as listed in FIG. 47 and in FIG. 1 for schizophrenia. The composition can comprise a substantially enriched population of exosomes, wherein the population of exosomes is substantially homogeneous for schizophrenia specific exosomes or exosomes comprising one or more schizophrenia specific biomarkers, such as listed in FIG. 47 and in FIG. 1 for schizophrenia.

One or more schizophrenia specific biomarkers, such as listed in FIG. 47 and in FIG. 1 for schizophrenia, can also be detected by one or more systems disclosed herein, for characterizing a schizophrenia. For example, a detection system can comprise one or more probes to detect one or more schizophrenia specific biomarkers, such as listed in FIG. 47 and in FIG. 1 for schizophrenia, of one or more exosomes of a biological sample.

Bipolar Disease

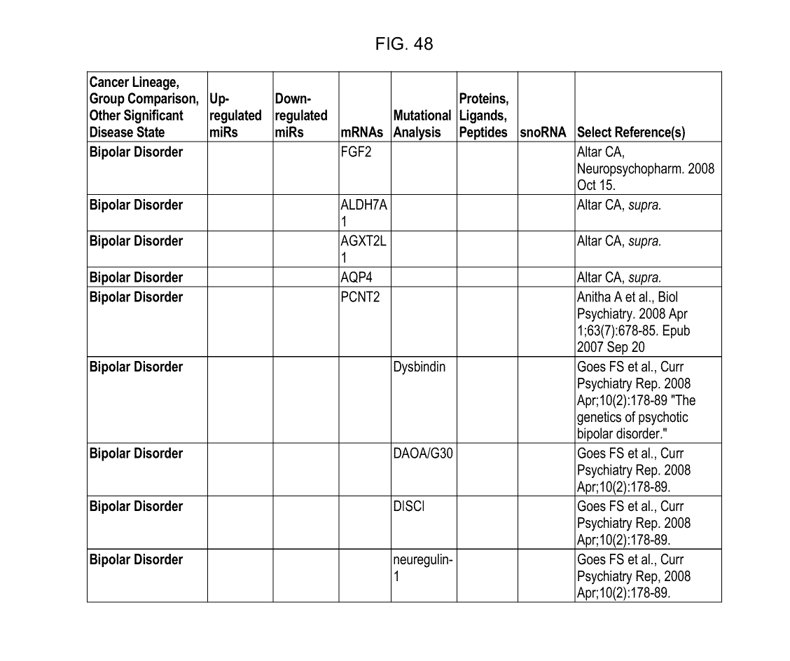

Bipolar disease specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 48, and can be used to create a bipolar disease specific exosome bio-signature. For example, the one or more mRNAs that may be analyzed can include, but are not limited to, FGF2, ALDH7A1, AGXT2L1, AQP4, or PCNT2, or any combination thereof and can be used as specific biomarkers from exosomes for bipolar disease. A biomarker mutation for bipolar disease that can be assessed in an exosome includes, but is not limited to, a mutation of Dysbindin, DAOA/G30, DISC1, neuregulin-1, or any combination of mutations specific for bipolar disease.

Also provided herein is an isolated exosome comprising one or more bipolar disease specific biomarkers, such as listed in FIG. 48. A composition comprising the isolated exosome is also provided. Accordingly, in some embodiments, the composition comprises a population of exosomes comprising one or more bipolar disease specific biomarkers, such as listed in FIG. 48. The composition can comprise a substantially enriched population of exosomes, wherein the population of exosomes is substantially homogeneous for bipolar disease specific exosomes or exosomes comprising one or more bipolar disease specific biomarkers, such as listed in FIG. 48.

One or more bipolar disease specific biomarkers, such as listed in FIG. 48, can also be detected by one or more systems disclosed herein, for characterizing a bipolar disease. For example, a detection system can comprise one or more probes to detect one or more bipolar disease specific biomarkers, such as listed in FIG. 48, of one or more exosomes of a biological sample.

Depression

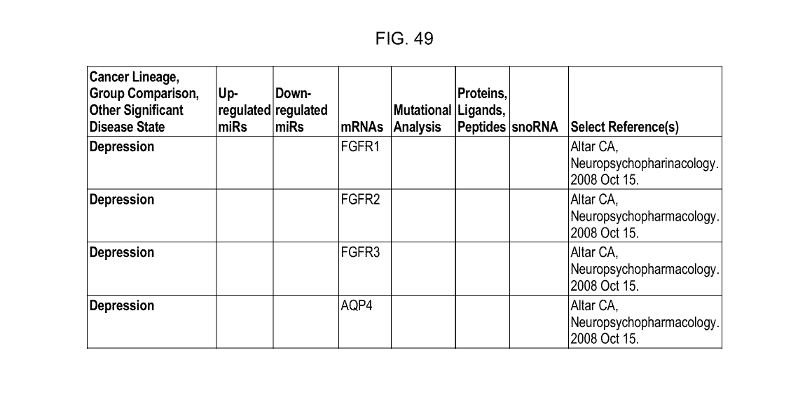

Depression specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 49, and can be used to create a depression specific exosome bio-signature. For example, the one or more mRNAs that may be analyzed can include, but are not limited to, FGFR1, FGFR2, FGFR3, or AQP4, or any combination thereof can also be used as specific biomarkers from exosomes for depression.

Also provided herein is an isolated exosome comprising one or more depression specific biomarkers, such as listed in FIG. 49. A composition comprising the isolated exosome is also provided. Accordingly, in some embodiments, the composition comprises a population of exosomes comprising one or more depression specific biomarkers, such as listed in FIG. 49. The composition can comprise a substantially enriched population of exosomes, wherein the population of exosomes is substantially homogeneous for depression specific exosomes or exosomes comprising one or more depression specific biomarkers, such as listed in FIG. 49.

One or more depression specific biomarkers, such as listed in FIG. 49, can also be detected by one or more systems disclosed herein, for characterizing a depression. For example, a detection system can comprise one or more probes to detect one or more depression specific biomarkers, such as listed in FIG. 49, of one or more exosomes of a biological sample.

Gastrointestinal Stromal Tumor (GIST)

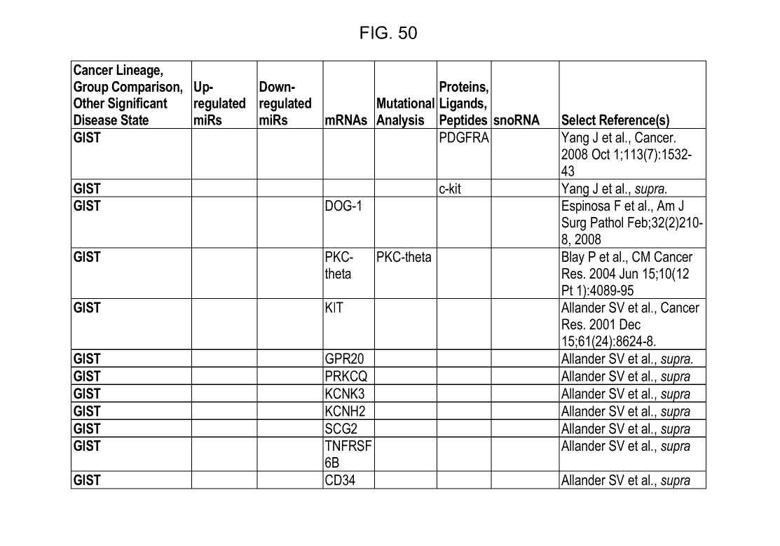

GIST specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 50, and can be used to create a GIST specific exosome bio-signature. For example, the one or more mRNAs that may be analyzed can include, but are not limited to, DOG-1, PKC-theta, KIT, GPR20, PRKCQ, KCNK3, KCNH2, SCG2, TNFRSF6B, or CD34, or any combination thereof and can be used as specific biomarkers from exosomes for GIST.

A biomarker mutation for GIST that can be assessed in an exosome includes, but is not limited to, a mutation of PKC-theta or any combination of mutations specific for GIST. The protein, ligand, or peptide that can be assessed in an exosome can include, but is not limited to, PDGFRA, c-kit, or any combination thereof.

Also provided herein is an isolated exosome comprising one or more GIST specific biomarkers, such as listed in FIG. 50 and in FIG. 1 for GIST. A composition comprising the isolated exosome is also provided. Accordingly, in some embodiments, the composition comprises a population of exosomes comprising one or more GIST specific biomarkers, such as listed in FIG. 50 and in FIG. 1 for GIST. The composition can comprise a substantially enriched population of exosomes, wherein the population of exosomes is substantially homogeneous for GIST specific exosomes or exosomes comprising one or more GIST specific biomarkers, such as listed in FIG. 50 and in FIG. 1 for GIST.

One or more GIST specific biomarkers, such as listed in FIG. 50 and in FIG. 1 for GIST, can also be detected by one or more systems disclosed herein, for characterizing a GIST. For example, a detection system can comprise one or more probes to detect one or more GIST specific biomarkers, such as listed in FIG. 50 and in FIG. 1 for GIST, of one or more exosomes of a biological sample.

Renal Cell Carcinoma

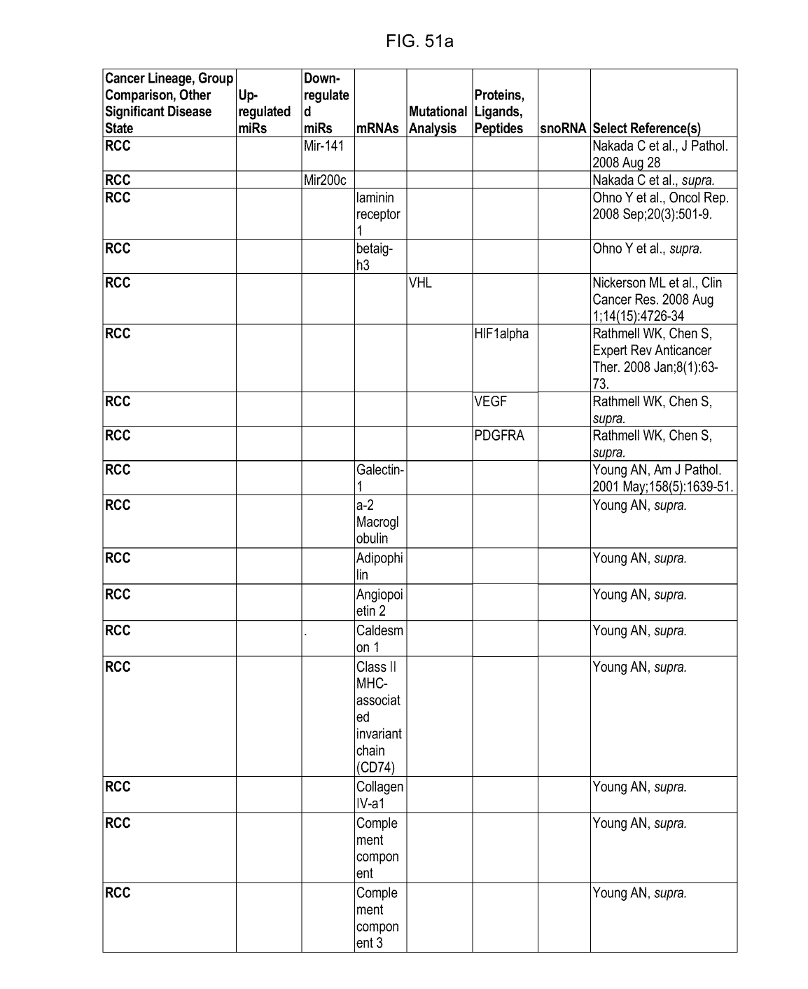

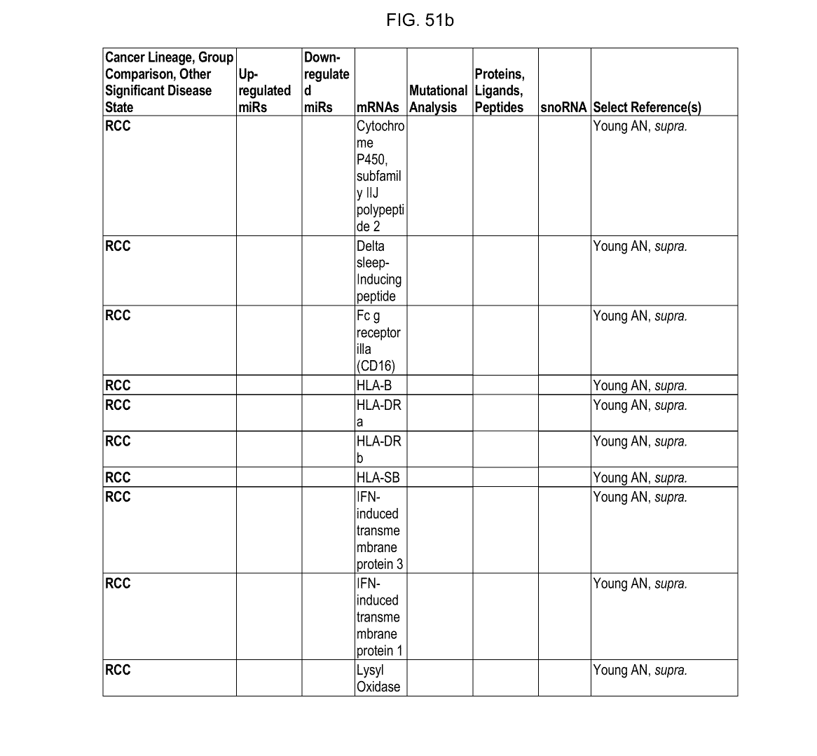

Renal cell carcinoma specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 51, and can be used to create a renal cell carcinoma specific exosome bio-signature. For example, the bio-signature can also comprise one or more underexpressed miRs such as, but not limited to, miR-141, miR-200c, or any combination thereof. The one or more upregulated or overexpressed miRNA can be miR-28, miR-185, miR-27, miR-let-7f-2, or any combination thereof.

The one or more mRNAs that may be analyzed can include, but are not limited to, laminin receptor 1, betaig-h3, Galectin-1, a-2 Macroglobulin, Adipophilin, Angiopoietin 2, Caldesmon 1, Class II MHC-associated invariant chain (CD74), Collagen IV-al, Complement component, Complement component 3, Cytochrome P450, subfamily IIJ polypeptide 2, Delta sleep-inducing peptide, Fc g receptor IIIa (CD16), HLA-B, HLA-DRa, HLA-DRb, HLA-SB, IFN-induced transmembrane protein 3, IFN-induced transmembrane protein 1, or Lysyl Oxidase, or any combination thereof and can be used as specific biomarkers from exosomes for renal cell carcinoma.

A biomarker mutation for renal cell carcinoma that can be assessed in an exosome includes, but is not limited to, a mutation of VHL or any combination of mutations specific renal cell carcinoma.

The protein, ligand, or peptide that can be assessed in an exosome can include, but is not limited to, IFlalpha, VEGF, PDGFRA, or any combination thereof.

Also provided herein is an isolated exosome comprising one or more RCC specific biomarkers, such as ALPHA-TFEB, NONO-TFE3, PRCC-TFE3, SFPQ-TFE3, CLTC-TFE3, or MALAT1-TFEB, or those listed in FIG. 51 and in FIG. 1 for RCC. A composition comprising the isolated exosome is also provided. Accordingly, in some embodiments, the composition comprises a population of exosomes comprising one or more RCC specific biomarkers, such as ALPHA-TFEB, NONO-TFE3, PRCC-TFE3, SFPQ-TFE3, CLTC-TFE3, or MALAT1-TFE, or those listed in FIG. 51 and in FIG. 1 for RCC. The composition can comprise a substantially enriched population of exosomes, wherein the population of exosomes is substantially homogeneous for RCC specific exosomes or exosomes comprising one or more RCC specific biomarkers, such as ALPHA-TFEB, NONO-TFE3, PRCC-TFE3, SFPQ-TFE3, CLTC-TFE3, or MALAT1-TFE, or those listed in FIG. 51 and in FIG. 1 for RCC.

One or more RCC specific biomarkers, such as ALPHA-TFEB, NONO-TFE3, PRCC-TFE3, SFPQ-TFE3, CLTC-TFE3, or MALAT1-TFE, or those listed in FIG. 51 and in FIG. 1 for RCC, can also be detected by one or more systems disclosed herein, for characterizing a RCC. For example, a detection system can comprise one or more probes to detect one or more RCC specific biomarkers, such as ALPHA-TFEB, NONO-TFE3, PRCC-TFE3, SFPQ-TFE3, CLTC-TFE3, or MALAT1-TFE, or those listed in FIG. 51 and in FIG. 1 for RCC, of one or more exosomes of a biological sample.

Cirrhosis

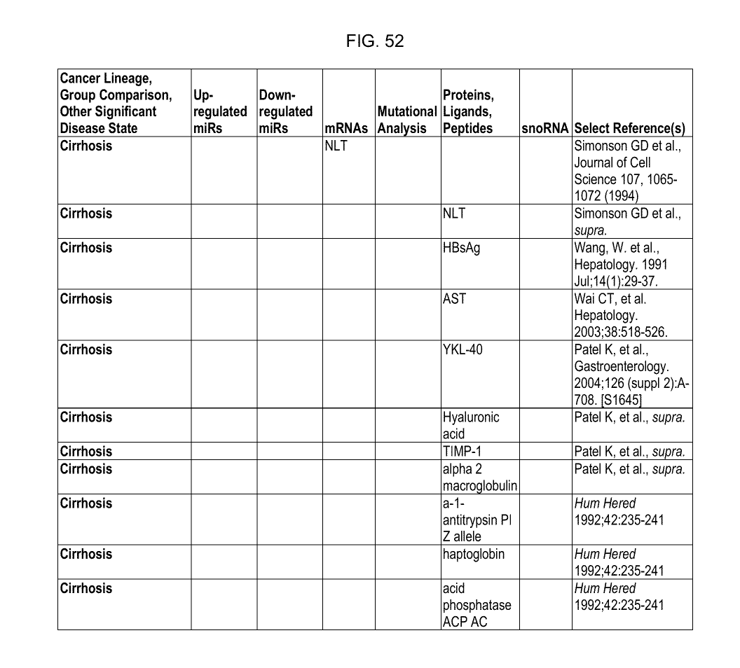

Cirrhosis specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 52, and can be used to create a cirrhosis specific exosome bio-signature. The one or more mRNAs that may be analyzed include, but are not limited to, NLT, which can be used as aspecific biomarker from exosomes for cirrhosis.

The protein, ligand, or peptide that can be assessed in an exosome can include, but is not limited to, NLT, HBsAG, AST, YKL-40, Hyaluronic acid, TIMP-1, alpha 2 macroglobulin, a-1-antitrypsin P1Z allele, haptoglobin, or acid phosphatase ACP AC, or any combination thereof.

Also provided herein is an isolated exosome comprising one or more cirrhosis specific biomarkers, such as those listed in FIG. 52 and in FIG. 1 for cirrhosis. A composition comprising the isolated exosome is also provided. Accordingly, in some embodiments, the composition comprises a population of exosomes comprising one or more cirrhosis specific biomarkers, such as those listed in FIG. 52 and in FIG. 1 for cirrhosis. The composition can comprise a substantially enriched population of exosomes, wherein the population of exosomes is substantially homogeneous for cirrhosis specific exosomes or exosomes comprising one or more cirrhosis specific biomarkers, such as those listed in FIG. 52 and in FIG. 1 for cirrhosis.

One or more cirrhosis specific biomarkers, such as those listed in FIG. 52 and in FIG. 1 for cirrhosis, can also be detected by one or more systems disclosed herein, for characterizing cirrhosis. For example, a detection system can comprise one or more probes to detect one or more cirrhosis specific biomarkers, such as those listed in FIG. 52 and in FIG. 1 for cirrhosis, of one or more exosomes of a biological sample.

Esophageal Cancer

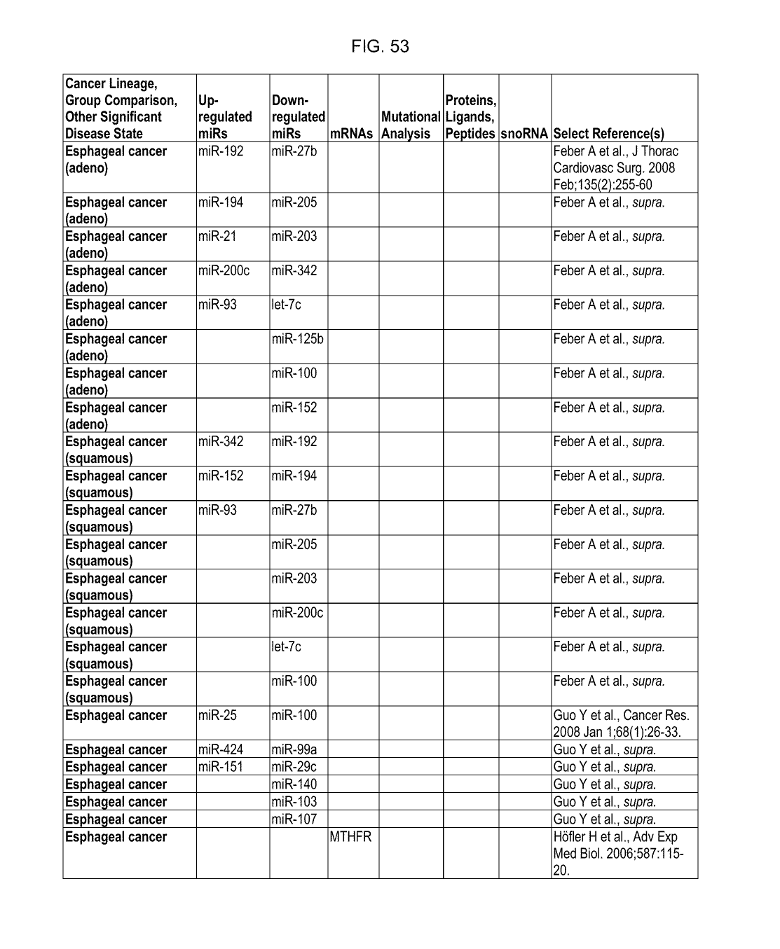

Esophageal cancer specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 53, and can be used to create a esophageal cancer specific exosome bio-signature. For example, the bio-signature can comprise one or more overexpressed miRs, such as, but not limited to, miR-192, miR-194, miR-21, miR-200c, miR-93, miR-342, miR-152, miR-93, miR-25, miR-424, or miR-151, or any combination thereof. The bio-signature can also comprise one or more underexpressed miRs such as, but not limited to, miR-27b, miR-205, miR-203, miR-342, let-7c, miR-125b, miR-100, miR-152, miR-192, miR-194, miR-27b, miR-205, miR-203, miR-200c, miR-99a, miR-29c, miR-140, miR-103, or miR-107, or any combination thereof. The one or more mRNAs that may be analyzed include, but are not limited to, MTHFR and can be used as specific biomarkers from exosomes for esophageal cancer.

Also provided herein is an isolated exosome comprising one or more esophageal cancer specific biomarkers, such as listed in FIG. 53 and in FIG. 1 for esophageal cancer. A composition comprising the isolated exosome is also provided. Accordingly, in some embodiments, the composition comprises a population of exosomes comprising one or more esophageal cancer specific biomarkers, such as listed in

FIG. 53 and in FIG. 1 for esophageal cancer. The composition can comprise a substantially enriched population of exosomes, wherein the population of exosomes is substantially homogeneous for esophageal cancer specific exosomes or exosomes comprising one or more esophageal cancer specific biomarkers, such as listed in FIG. 53 and in FIG. 1 for esophageal cancer.

One or more esophageal cancer specific biomarkers, such as listed in FIG. 53 and in FIG. 1 for esophageal cancer, can also be detected by one or more systems disclosed herein, for characterizing a esophageal cancer. For example, a detection system can comprise one or more probes to detect one or more esophageal cancer specific biomarkers, such as listed in FIG. 53 and in FIG. 1 for esophageal cancer, of one or more exosomes of a biological sample.

Gastric Cancer

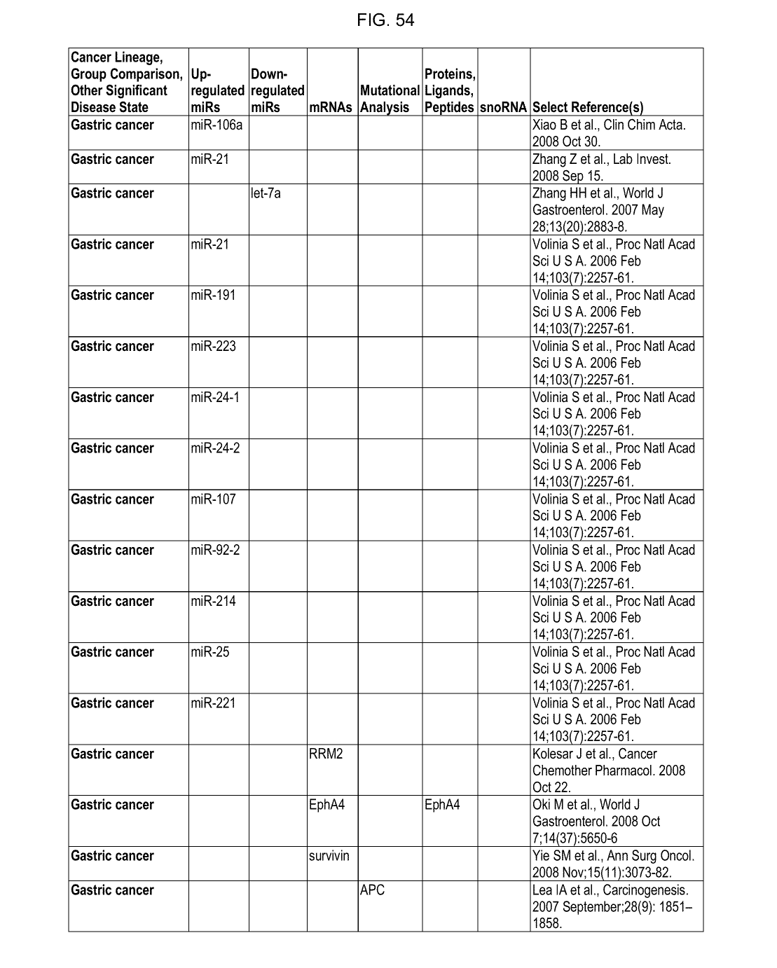

Gastric cancer specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 54, and can be used to create a gastric cancer specific exosome bio-signature. For example, the bio-signature can comprise one or more overexpressed miRs, such as, but not limited to, miR-106a, miR-21, miR-191, miR-223, miR-24-1, miR-24-2, miR-107, miR-92-2, miR-214, miR-25, or miR-221, or any combination thereof. The bio-signature can also comprise one or more underexpressed miRs such as, but not limited to, let-7a.

The one or more mRNAs that may be analyzed include, but are not limited to, RRM2, EphA4, or survivin, or any combination thereof and can be used as specific biomarkers from exosomes for gastric cancer. A biomarker mutation for gastric cancer that can be assessed in an exosome includes, but is not limited to, a mutation of APC or any combination of mutations specific for gastric cancer. The protein, ligand, or peptide that can be assessed in an exosome can include, but is not limited to EphA4.

Also provided herein is an isolated exosome comprising one or more gastric cancer specific biomarkers, such as listed in FIG. 54. A composition comprising the isolated exosome is also provided. Accordingly, in some embodiments, the composition comprises a population of exosomes comprising one or more gastric cancer specific biomarkers, such as listed in FIG. 54. The composition can comprise a substantially enriched population of exosomes, wherein the population of exosomes is substantially homogeneous for gastric cancer specific exosomes or exosomes comprising one or more gastric cancer specific biomarkers, such as listed in FIG. 54.

One or more gastric cancer specific biomarkers, such as listed in FIG. 54, can also be detected by one or more systems disclosed herein, for characterizing a gastric cancer. For example, a detection system can comprise one or more probes to detect one or more gastric cancer specific biomarkers, such as listed in FIG. 54, of one or more exosomes of a biological sample.

Autism

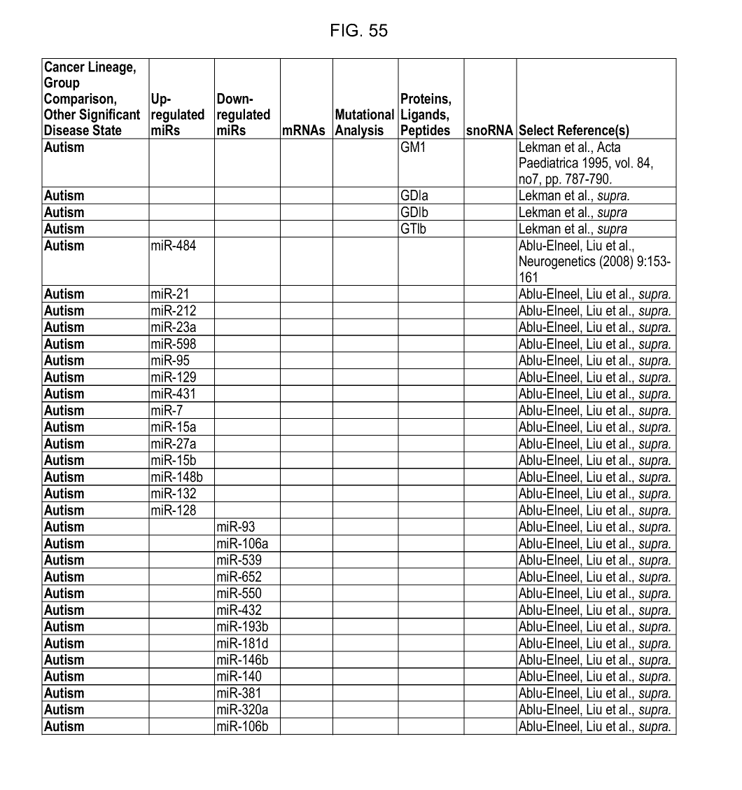

Autism specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 55, and can be used to create an autism specific exosome bio-signature. For example, the bio-signature can comprise one or more overexpressed miRs, such as, but not limited to, miR-484, miR-21, miR-212, miR-23a, miR-598, miR-95, miR-129, miR-431, miR-7, miR-15a, miR-27a, miR-15b, miR-148b, miR-132, or miR-128, or any combination thereof. The bio-signature can also comprise one or more underexpressed miRs such as, but not limited to, miR-93, miR-106a, miR-539, miR-652, miR-550, miR-432, miR-193b, miR-181d, miR-146b, miR-140, miR-381, miR-320a, or miR-106b, or any combination thereof. The protein, ligand, or peptide that can be assessed in an exosome can include, but is not limited to, GM1, GD1a, GD1b, or GT1b, or any combination thereof.

Also provided herein is an isolated exosome comprising one or more autism specific biomarkers, such as listed in FIG. 55 and in FIG. 1 for autism. A composition comprising the isolated exosome is also provided. Accordingly, in some embodiments, the composition comprises a population of exosomes comprising one or more autism specific biomarkers, such as listed in FIG. 55 and in FIG. 1 for autism. The composition can comprise a substantially enriched population of exosomes, wherein the population of exosomes is substantially homogeneous for autism specific exosomes or exosomes comprising one or more autism specific biomarkers, such as listed in FIG. 55 and in FIG. 1 for autism.

One or more autism specific biomarkers, such as listed in FIG. 55 and in FIG. 1 for autism, can also be detected by one or more systems disclosed herein, for characterizing a autism. For example, a detection system can comprise one or more probes to detect one or more autism specific biomarkers, such as listed in FIG. 55 and in FIG. 1 for autism, of one or more exosomes of a biological sample.

Organ Rejection

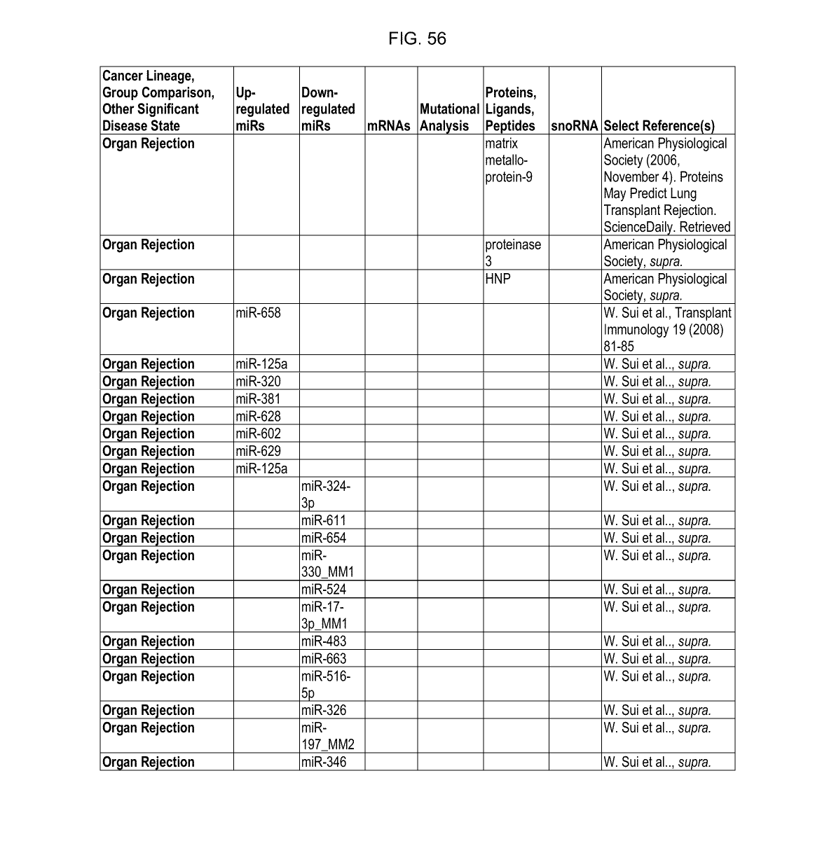

Organ rejection specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 56, and can be used to create an organ rejection specific exosome bio-signature. For example, the bio-signature can comprise one or more overexpressed miRs, such as, but not limited to, miR-658, miR-125a, miR-320, miR-381, miR-628, miR-602, miR-629, or miR-125a, or any combination thereof. The bio-signature can also comprise one or more underexpressed miRs such as, but not limited to, miR-324-3p, miR-611, miR-654, miR-330_MM1, miR-524, miR-17-3p_MM1, miR-483, miR-663, miR-516-5p, miR-326, miR-197_MM2, or miR-346, or any combination thereof. The protein, ligand, or peptide that can be assessed in an exosome can include, but is not limited to, matix metalloprotein-9, proteinase 3, or HNP, or any combinations thereof. The biomarker can be a member of the matrix metalloproteinases.

Also provided herein is an isolated exosome comprising one or more organ rejection specific biomarkers, such as listed in FIG. 56. A composition comprising the isolated exosome is also provided. Accordingly, in some embodiments, the composition comprises a population of exosomes comprising one or more organ rejection specific biomarkers, such as listed in FIG. 56. The composition can comprise a substantially enriched population of exosomes, wherein the population of exosomes is substantially homogeneous for organ rejection specific exosomes or exosomes comprising one or more organ rejection specific biomarkers, such as listed in FIG. 56.

One or more organ rejection specific biomarkers, such as listed in FIG. 56, can also be detected by one or more systems disclosed herein, for characterizing a organ rejection. For example, a detection system can comprise one or more probes to detect one or more organ rejection specific biomarkers, such as listed in FIG. 56, of one or more exosomes of a biological sample.

Methicillin-Resistant Staphylococcus aureus

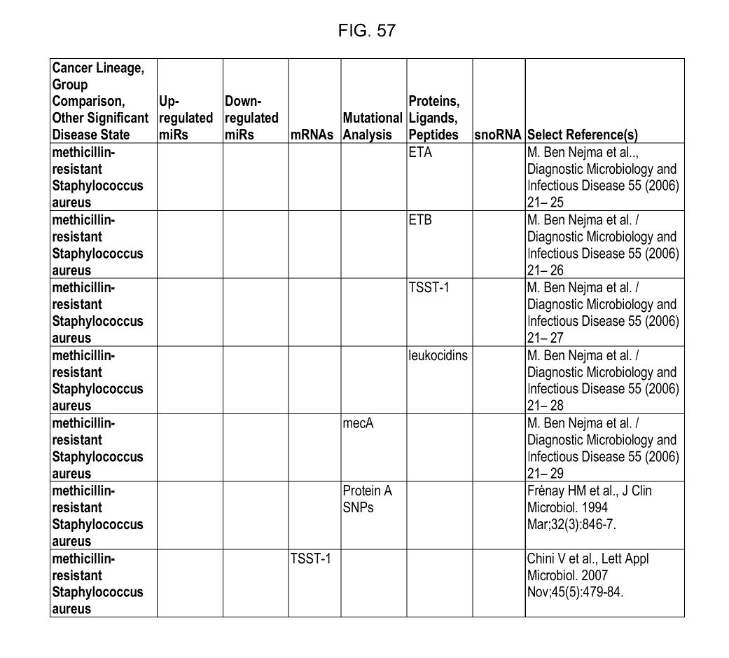

Methicillin-resistant Staphylococcus aureus specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 57, and can be used to create a methicillin-resistant Staphylococcus aureus specific exosome bio-signature.

The one or more mRNAs that may be analyzed include, but are not limited to, TSST-1 which can be used as a specific biomarker from exosomes for methicillin-resistant Staphylococcus aureus. A biomarker mutation for methicillin-resistant Staphylococcus aureus that can be assessed in an exosome includes, but is not limited to, a mutation of mecA, Protein A SNPs, or any combination of mutations specific for methicillin-resistant Staphylococcus aureus. The protein, ligand, or peptide that can be assessed in an exosome can include, but is not limited to, ETA, ETB, TSST-1, or leukocidins, or any combination thereof.

Also provided herein is an isolated exosome comprising one or more methicillin-resistant Staphylococcus aureus specific biomarkers, such as listed in FIG. 57. A composition comprising the isolated exosome is also provided. Accordingly, in some embodiments, the composition comprises a population of exosomes comprising one or more methicillin-resistant Staphylococcus aureus specific biomarkers, such as listed in FIG. 57. The composition can comprise a substantially enriched population of exosomes, wherein the population of exosomes is substantially homogeneous for methicillin-resistant Staphylococcus aureus specific exosomes or exosomes comprising one or more methicillin-resistant Staphylococcus aureus specific biomarkers, such as listed in FIG. 57.

One or more methicillin-resistant Staphylococcus aureus specific biomarkers, such as listed in FIG. 57, can also be detected by one or more systems disclosed herein, for characterizing a methicillin-resistant Staphylococcus aureus. For example, a detection system can comprise one or more probes to detect one or more methicillin-resistant Staphylococcus aureus specific biomarkers, such as listed in FIG. 57, of one or more exosomes of a biological sample.

Vulnerable Plaque

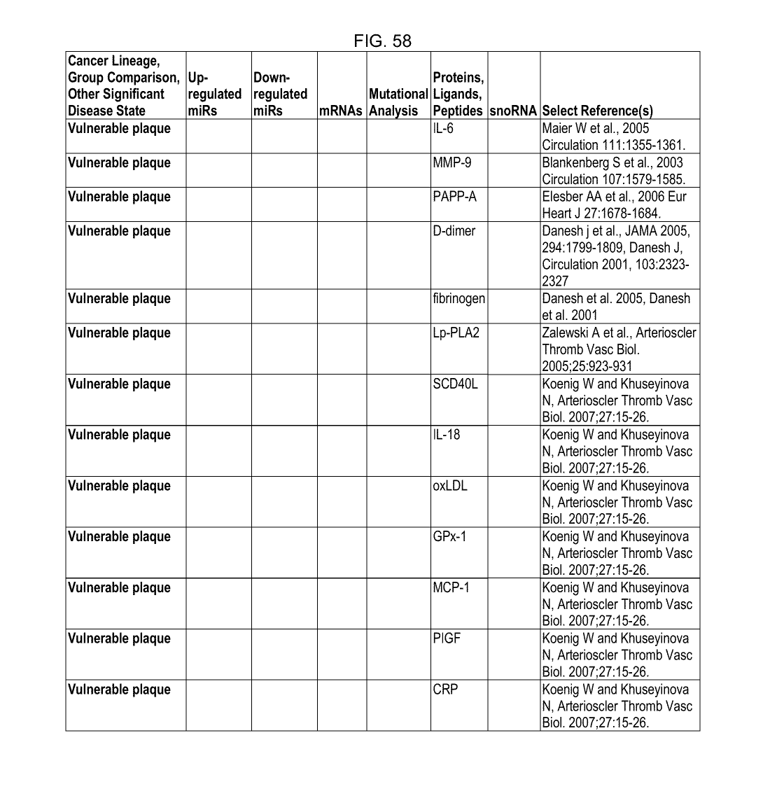

Vulnerable plaque specific biomarkers from exosomes can include one or more (for example, 2, 3, 4, 5, 6, 7, 8, or more) overexpressed miRs, underexpressed miRs, mRNAs, genetic mutations, proteins, ligands, peptides, snoRNA, or any combination thereof, such as listed in FIG. 58, and can be used to create a vulnerable plaque specific exosome bio-signature. The protein, ligand, or peptide that can be assessed in an exosome can include, but is not limited to, IL-6, MMP-9, PAPP-A, D-dimer, fibrinogen, Lp-PLA2, SCD40L, Il-18, oxLDL, GPx-1, MCP-1, PIGF, or CRP, or any combination thereof.

Also provided herein is an isolated exosome comprising one or more vulnerable plaque specific biomarkers, such as listed in FIG. 58 and in FIG. 1 for vulnerable plaque. A composition comprising the isolated exosome is also provided. Accordingly, in some embodiments, the composition comprises a population of exosomes comprising one or more vulnerable plaque specific biomarkers, such as listed in FIG. 58 and in FIG. 1 for vulnerable plaque. The composition can comprise a substantially enriched population of exosomes, wherein the population of exosomes is substantially homogeneous for vulnerable plaque specific exosomes or exosomes comprising one or more vulnerable plaque specific biomarkers, such as listed in FIG. 58 and in FIG. 1 for vulnerable plaque.

One or more vulnerable plaque specific biomarkers, such as listed in FIG. 58 and in FIG. 1 for vulnerable plaque, can also be detected by one or more systems disclosed herein, for characterizing a vulnerable plaque. For example, a detection system can comprise one or more probes to detect one or more vulnerable plaque specific biomarkers, such as listed in FIG. 58 and in FIG. 1 for vulnerable plaque, of one or more exosomes of a biological sample.

Autoimmune Disease

Also provided herein is an isolated exosome comprising one or more autoimmune disease specific biomarkers, such as listed in FIG. 1 for autoimmune disease. A composition comprising the isolated exosome is also provided. Accordingly, in some embodiments, the composition comprises a population of exosomes comprising one or more autoimmune disease specific biomarkers, such as listed in FIG. 1 for autoimmune disease. The composition can comprise a substantially enriched population of exosomes, wherein the population of exosomes is substantially homogeneous for autoimmune disease specific exosomes or exosomes comprising one or more autoimmune disease specific biomarkers, such as listed in FIG. 1 for autoimmune disease.

One or more autoimmune disease specific biomarkers, such as listed in FIG. 1 for autoimmune disease, can also be detected by one or more systems disclosed herein, for characterizing a autoimmune disease. For example, a detection system can comprise one or more probes to detect one or more autoimmune disease specific biomarkers, such as listed in FIG. 1 for autoimmune disease, of one or more exosomes of a biological sample.

Tuberculosis (TB)

Also provided herein is an isolated exosome comprising one or more TB disease specific biomarkers, such as listed in FIG. 1 for TB disease. A composition comprising the isolated exosome is also provided. Accordingly, in some embodiments, the composition comprises a population of exosomes comprising one or more TB disease specific biomarkers, such as listed in FIG. 1 for TB disease. The composition can comprise a substantially enriched population of exosomes, wherein the population of exosomes is substantially homogeneous for TB disease specific exosomes or exosomes comprising one or more TB disease specific biomarkers, such as listed in FIG. 1 for TB disease.

One or more TB disease specific biomarkers, such as listed in FIG. 1 for TB disease, can also be detected by one or more systems disclosed herein, for characterizing a TB disease. For example, a detection system can comprise one or more probes to detect one or more TB disease specific biomarkers, such as listed in FIG. 1 for TB disease, of one or more exosomes of a biological sample.

HIV

Also provided herein is an isolated exosome comprising one or more HIV disease specific biomarkers, such as listed in FIG. 1 for HIV disease. A composition comprising the isolated exosome is also provided. Accordingly, in some embodiments, the composition comprises a population of exosomes comprising one or more HIV disease specific biomarkers, such as listed in FIG. 1 for HIV disease. The composition can comprise a substantially enriched population of exosomes, wherein the population of exosomes is substantially homogeneous for HIV disease specific exosomes or exosomes comprising one or more HIV disease specific biomarkers, such as listed in FIG. 1 for HIV disease.

One or more HIV disease specific biomarkers, such as listed in FIG. 1 for HIV disease, can also be detected by one or more systems disclosed herein, for characterizing a HIV disease. For example, a detection system can comprise one or more probes to detect one or more HIV disease specific biomarkers, such as listed in FIG. 1 for HIV disease, of one or more exosomes of a biological sample.

The one or more biomarker can also be a miRNA, such as an upregulated or overexpressed miRNA. The upregulated miRNA can be miR-29a, miR-29a, miR-149, miR-378 or miR-324-5p. One or more biomarkers can also be used to charcterize HIV-1 latency, such as by assessing one or more miRNAs. The miRNA can be miR-28, miR-125b, miR-150, miR-223 and miR-382, and upregulated.

Asthma

Also provided herein is an isolated exosome comprising one or more asthma disease specific biomarkers, such as listed in FIG. 1 for asthma disease. A composition comprising the isolated exosome is also provided. Accordingly, in some embodiments, the composition comprises a population of exosomes comprising one or more asthma disease specific biomarkers, such as listed in FIG. 1 for asthma disease. The composition can comprise a substantially enriched population of exosomes, wherein the population of exosomes is substantially homogeneous for asthma disease specific exosomes or exosomes comprising one or more asthma disease specific biomarkers, such as listed in FIG. 1 for asthma disease.

One or more asthma disease specific biomarkers, such as listed in FIG. 1 for asthma disease, can also be detected by one or more systems disclosed herein, for characterizing a asthma disease. For example, a detection system can comprise one or more probes to detect one or more asthma disease specific biomarkers, such as listed in FIG. 1 for asthma disease, of one or more exosomes of a biological sample.

Lupus

Also provided herein is an isolated exosome comprising one or more lupus disease specific biomarkers, such as listed in FIG. 1 for lupus disease. A composition comprising the isolated exosome is also provided. Accordingly, in some embodiments, the composition comprises a population of exosomes comprising one or more lupus disease specific biomarkers, such as listed in FIG. 1 for lupus disease. The composition can comprise a substantially enriched population of exosomes, wherein the population of exosomes is substantially homogeneous for lupus disease specific exosomes or exosomes comprising one or more lupus disease specific biomarkers, such as listed in FIG. 1 for lupus disease.

One or more lupus disease specific biomarkers, such as listed in FIG. 1 for lupus disease, can also be detected by one or more systems disclosed herein, for characterizing a lupus disease. For example, a detection system can comprise one or more probes to detect one or more lupus disease specific biomarkers, such as listed in FIG. 1 for lupus disease, of one or more exosomes of a biological sample.

Influenza

Also provided herein is an isolated exosome comprising one or more influenza disease specific biomarkers, such as listed in FIG. 1 for influenza disease. A composition comprising the isolated exosome is also provided. Accordingly, in some embodiments, the composition comprises a population of exosomes comprising one or more influenza disease specific biomarkers, such as listed in FIG. 1 for influenza disease. The composition can comprise a substantially enriched population of exosomes, wherein the population of exosomes is substantially homogeneous for influenza disease specific exosomes or exosomes comprising one or more influenza disease specific biomarkers, such as listed in FIG. 1 for influenza disease.

One or more influenza disease specific biomarkers, such as listed in FIG. 1 for influenza disease, can also be detected by one or more systems disclosed herein, for characterizing a influenza disease. For example, a detection system can comprise one or more probes to detect one or more influenza disease specific biomarkers, such as listed in FIG. 1 for influenza disease, of one or more exosomes of a biological sample.

Thyroid Cancer

Also provided herein is an isolated exosome comprising one or more thyroid cancer specific biomarkers, such as AKAP9-BRAF, CCDC6-RET, ERC1-RET, GOLGA5-RET, HOOK3-RET, HRH4-RET, KTN1-RET, NCOA4-RET, PCM1-RET, PRKARA1A-RET, RFG-RET, RFG9-RET, Ria-RET, TGF-NTRK1, TPM3-NTRK1, TPM3-TPR, TPR-MET, TPR-NTRK1, TRIM24-RET, TRIM27-RET or TRIM33-RET, characteristic of papillary thyroid carcinoma; or PAX8-PPARy, characteristic of follicular thyroid cancer. A composition comprising the isolated exosome is also provided. Accordingly, in some embodiments, the composition comprises a population of exosomes comprising one or more thyroid cancer specific biomarkers, such as listed in FIG. 1 for thyroid cancer. The composition can comprise a substantially enriched population of exosomes, wherein the population of exosomes is substantially homogeneous for thyroid cancer specific exosomes or exosomes comprising one or more thyroid cancer specific biomarkers, such as listed in FIG. 1 for thyroid cancer.

One or more thyroid cancer specific biomarkers, such as listed in FIG. 1 for thyroid cancer, can also be detected by one or more systems disclosed herein, for characterizing a thyroid cancer. For example, a detection system can comprise one or more probes to detect one or more thyroid cancer specific biomarkers, such as listed in FIG. 1 for thyroid cancer, of one or more exosomes of a biological sample.

Gene Fusions

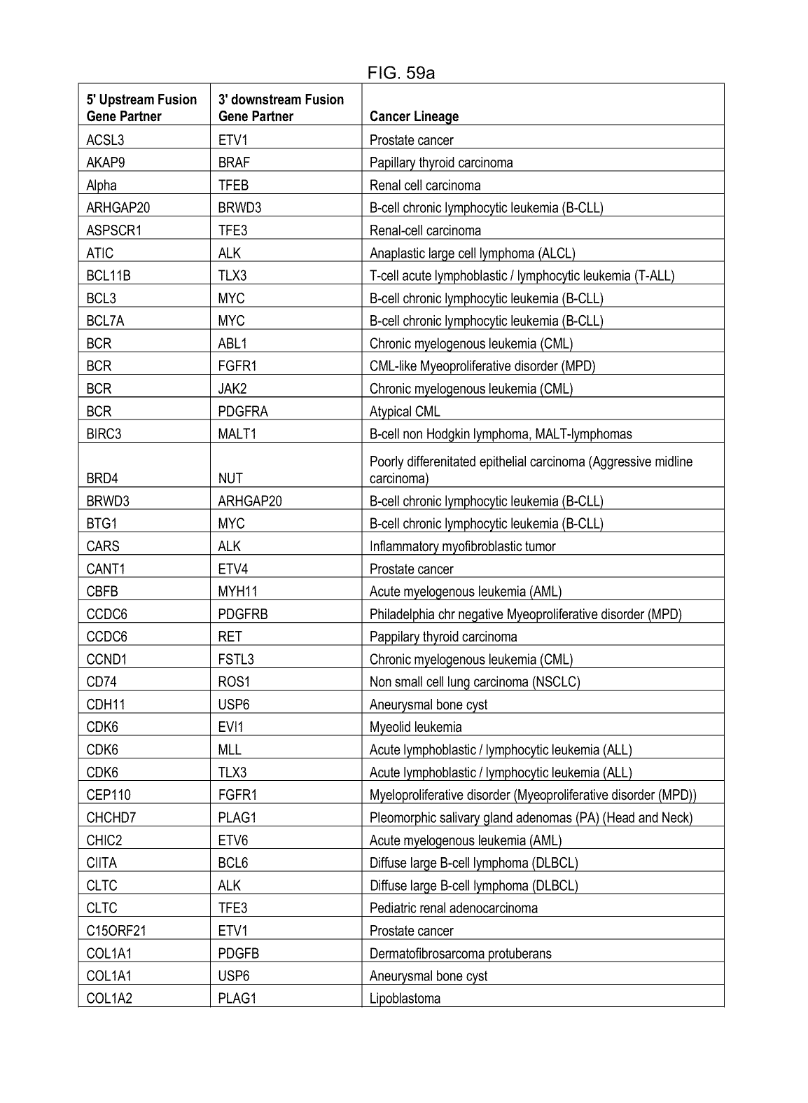

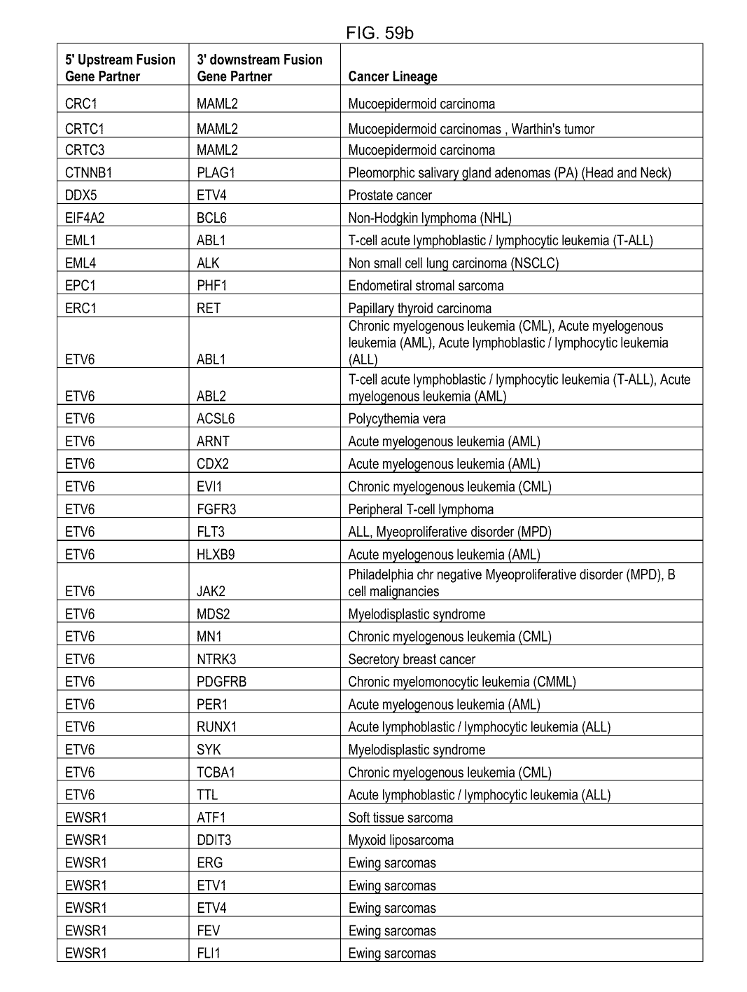

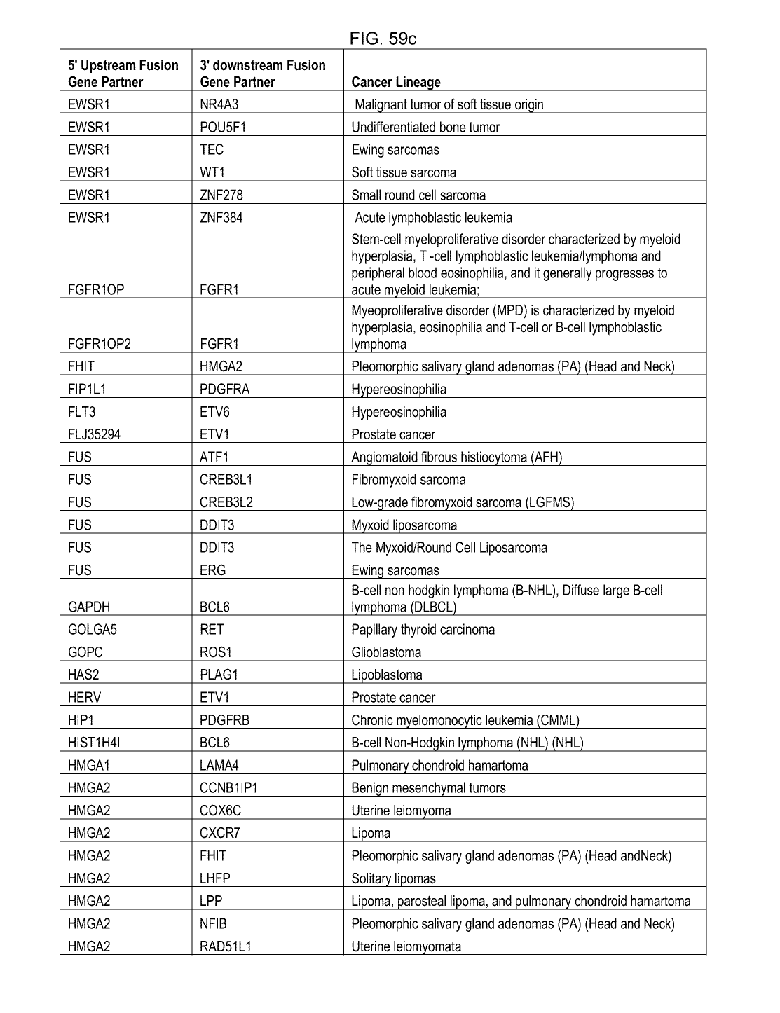

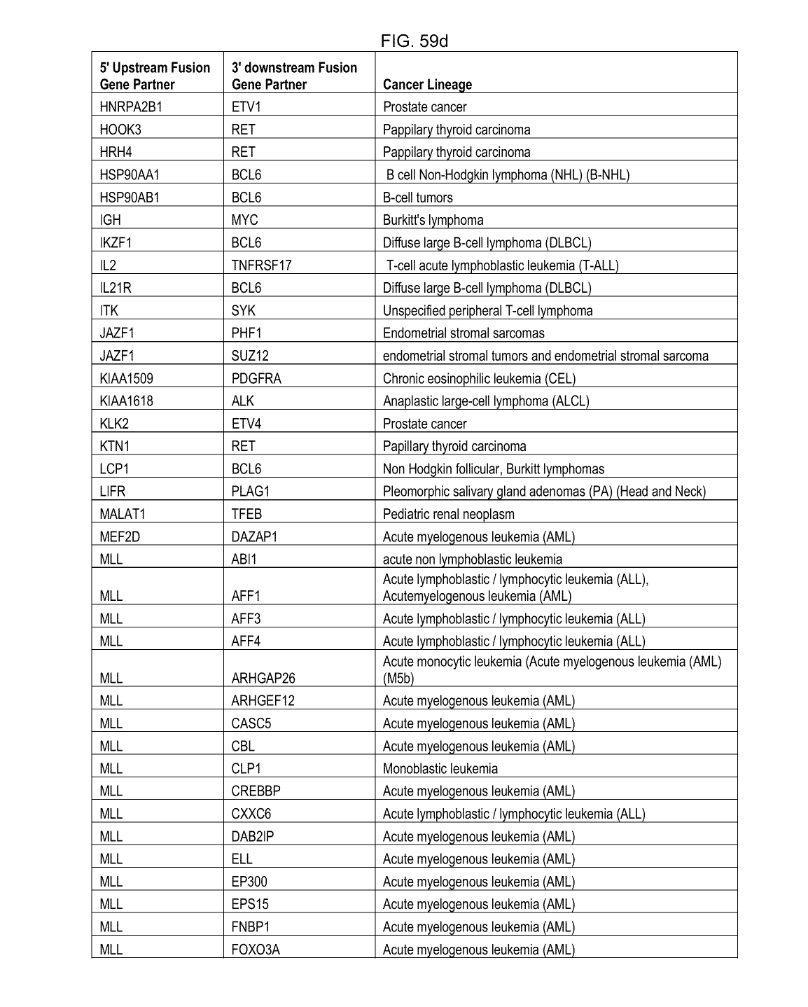

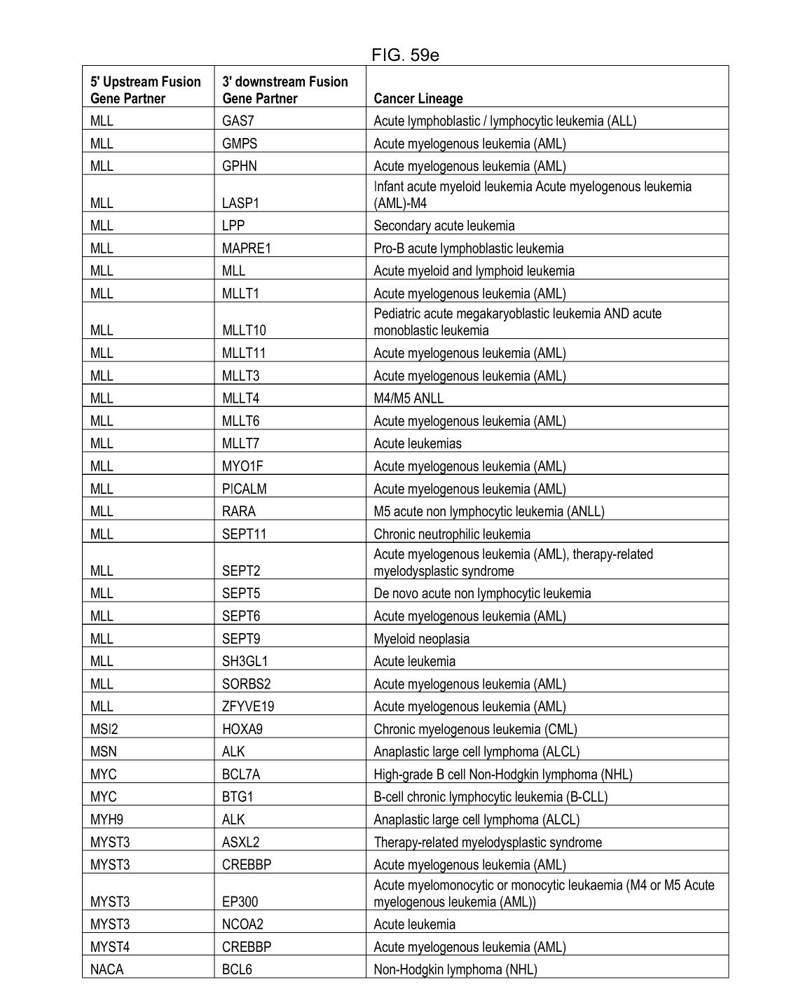

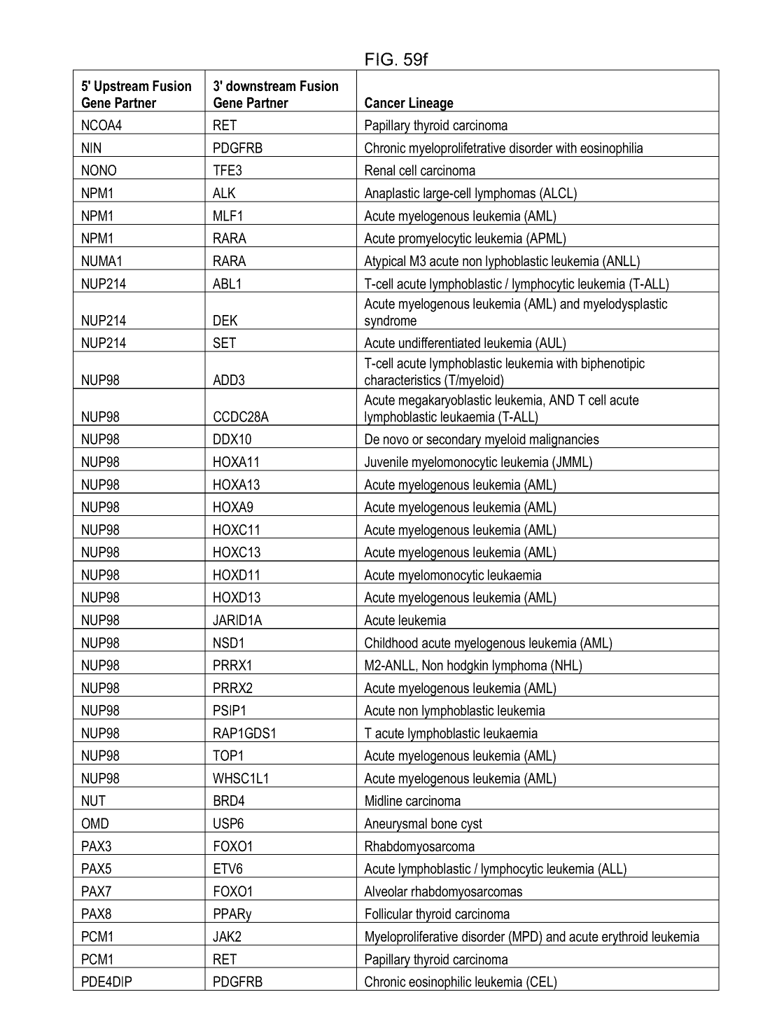

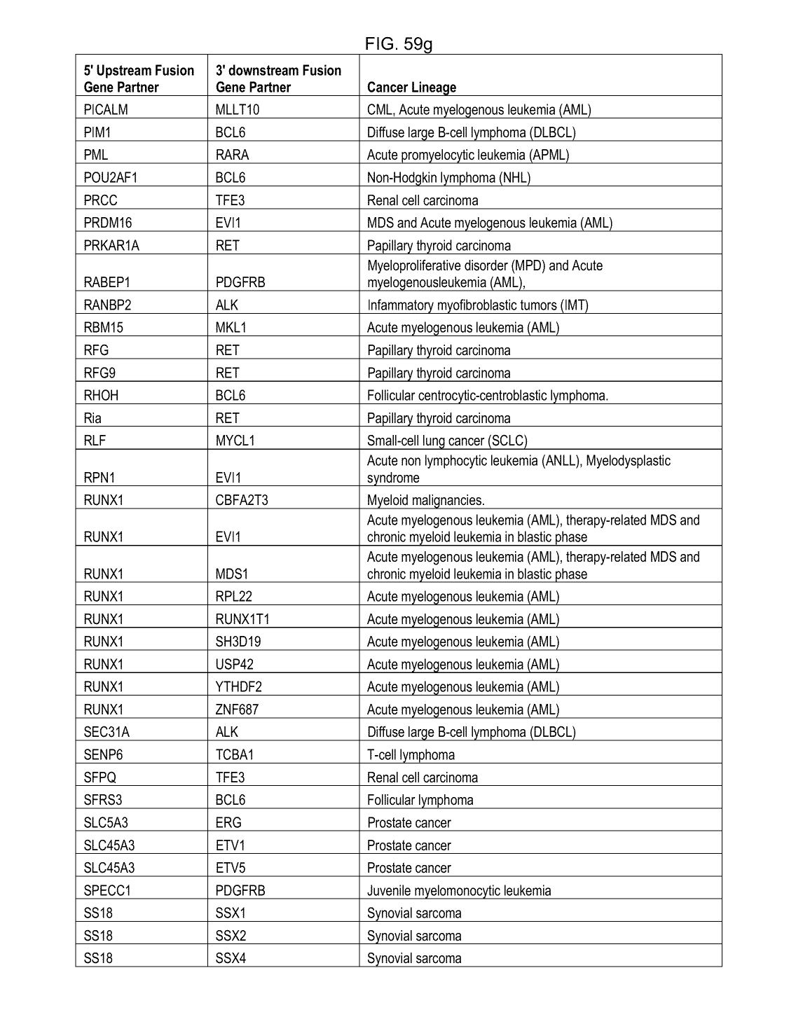

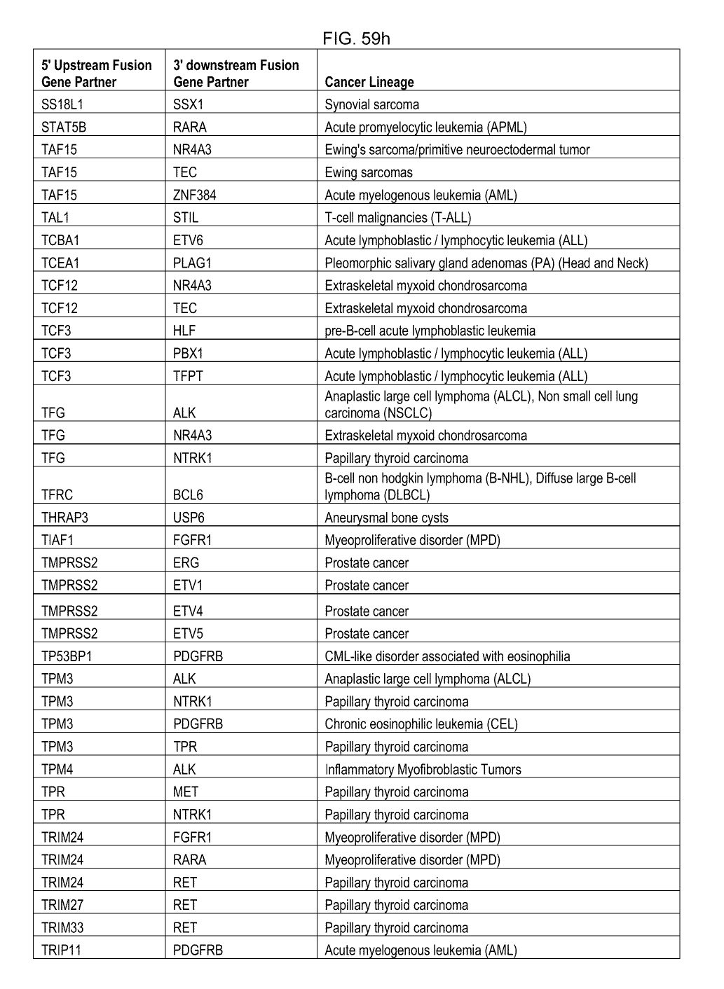

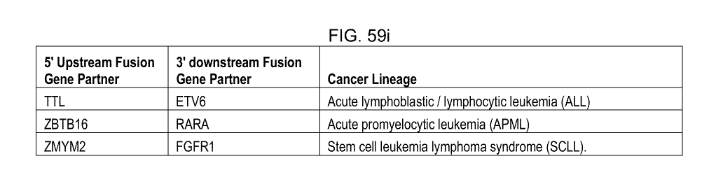

The one or more biomarkers assessed of an exosome can be a gene fusion, such as one or more listed in FIG. 59. A fusion gene is a hybrid gene created by the juxtaposition of two previously separate genes. This can occur by chromosomal translocation or inversion, deletion or via trans-splicing. The resulting fusion gene can cause abnormal temporal and spatial expression of genes, such as leading to abnormal expression ofcell growth factors, angiogenesis factors, tumor promoters or other factors contributing to the neoplastic transformation of the cell and the creation of a tumor. Such fusion genes can be oncogenic due to the juxtaposition of: 1) a strong promoter region of one gene next to the coding region of a cell growth factor, tumor promoter or other gene promoting oncogenesis leading to elevated gene expression, or 2) due to the fusion of coding regions of two different genes, giving rise to a chimeric gene and thus a chimeric protein with abnormal activity.

An example of a fusion gene is BCR-ABL, a characteristic molecular aberration in ˜90% of chronic myelogenous leukemia (CML) and in a subset of acute leukemias (Kurzrock et al., Annals of Internal Medicine 2003; 138(10):819-830). The BCR-ABL results from a translocation between chromosomes 9 and 22. The translocation brings together the 5′ region of the BCR gene and the 3′ region of ABL1, generating a chimeric BCR-ABL1 gene, which encodes a protein with constitutively active tyrosine kinase activity (Mittleman et al., Nature Reviews Cancer 2007; 7(4):233-245). The aberrant tyrosine kinase activity leads to de-regulated cell signaling, cell growth and cell survival, apoptosis resistance and growth factor independence, all of which contribute to the pathophysiology of leukemia (Kurzrock et al., Annals of Internal Medicine 2003; 138(10):819-830).

Another fusion gene is IGH-MYC, a defining feature of ˜80% of Burkitt's lymphoma (Ferry et al. Oncologist 2006; 11(4):375-83). The causal event for this is a translocation between chromosomes 8 and 14, bringing the c-Myc oncogene adjacent to the strong promoter of the immunoglobin heavy chain gene, causing c-myc overexpression (Mittleman et al., Nature Reviews Cancer 2007; 7(4):233-245). The c-myc rearrangement is a pivotal event in lymphomagenesis as it results in a perpetually proliferative state. It has wide ranging effects on progression through the cell cycle, cellular differentiation, apoptosis, and cell adhesion (Ferry et al. Oncologist 2006; 11(4):375-83).

A number of recurrent fusion genes have been catalogued in the Mittleman database (http://cgap.nci.nih.gov/Chromosomes/Mitelman) and can be assess in an exosome and used to characterize a phenotype. The gene fusion can be used to characterize a hematological malignancy or epithelial tumor. For example, TMPRSS2-ERG, TMPRSS2-ETV and SLC45A3-ELK4 fusions can be detected and used to characterize prostate cancer; and ETV6-NTRK3 and ODZ4-NRG1 for breast cancer.

Furthermore, assessing the presence or absence, or expression level of a fusion gene can be used to diagnosis a phenotype such as a cancer as well as a monitoring a therapeutic response to selecting a treatment. For example, the presence of the BCR-ABL fusion gene is a characteristic not only for the diagnosis of CML, but is also the target of the Novartis drug Imatinib mesylate (Gleevec), a receptor tyrosine kinase inhibitor, for the treatment of CML. Imatinib treatment has led to molecular responses (disappearance of BCR-ABL+blood cells) and improved progression-free survival in BCR-ABL+CML patients (Kantarjian et al., Clinical Cancer Research 2007; 13(4):1089-1097).

Assessing an exosome for the presence, absence, or expression level of a gene fusion can be of a heterogeneous population of exosomes. Alternatively, the exosome can be derived from a specific cell type, such as cell-or-origin specific exosomes, as described above.

Breast Cancer

To characterize a breast cancer, an exosome can be assessed for one or more breast cancer specific fusions, including, but not limited to, ETV6-NTRK3. The exosome can be derived from breast cancer cells.

Lung Cancer

To characterize a lung cancer, an exosome can be assessed for one or more lung cancer specific fusions, including, but not limited to, RLF-MYCL1, TGF-ALK, or CD74-ROS1. The exosome can be derived from lung cancer cells.

Prostate Cancer

To characterize a prostate cancer, an exosome can be assessed for one or more prostate cancer specific fusions, including, but not limited to, ACSL3-ETV1, C150RF21-ETV1, F1135294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4. The exosome can be derived from prostate cancer cells.

Brain Cancer

To characterize a brain cancer, an exosome can be assessed for one or more brain cancer specific fusions, including, but not limited to, GOPC-ROS1. The exosome can be derived from brain cancer cells.

Head and Neck Cancer

To characterize a head and neck cancer, an exosome can be assessed for one or more head and neck cancer specific fusions, including, but not limited to, CHCHD7-PLAG1, CTNNB1-PLAG1, FHIT-HMGA2, HMGA2-NFIB, LIFR-PLAG1, or TCEA1-PLAG1. The exosome can be derived from head and neck cancer cells.

Renal Cell Carcinoma (RCC)

To characterize a RCC, an exosome can be assessed for one or more RCC specific fusions, including, but not limited to, ALPHA-TFEB, NONO-TFE3, PRCC-TFE3, SFPQ-TFE3, CLTC-TFE3, or MALAT1-TFEB. The exosome can be derived from RCC cells.

Thyroid Cancer

To characterize a thyroid cancer, an exosome can be assessed for one or more thyroid cancer specific fusions, including, but not limited to, AKAP9-BRAF, CCDC6-RET, ERC1-RET, GOLGA5-RET, HOOK3-RET, HRH4-RET, KTN1-RET, NCOA4-RET, PCM1-RET, PRKARA1A-RET, RFG-RET, RFG9-RET, Ria-RET, TGF-NTRK1, TPM3-NTRK1, TPM3-TPR, TPR-MET, TPR-NTRK1, TRIM24-RET, TRIM27-RET or TRIM33-RET, characteristic of papillary thyroid carcinoma; or PAX8-PPARy, characteristic of follicular thyroid cancer. The exosome can be derived from thyroid cancer cells.

Blood Cancers

To characterize a blood cancer, an exosome can be assessed for one or more blood cancer specific fusions, including, but not limited to, TTL-ETV6, CDK6-MLL, CDK6-TLX3, ETV6-FLT3, ETV6-RUNX1, ETV6-TTL, MLL-AFF1, MLL-AFF3, MLL-AFF4, MLL-GAS7, TCBA1-ETV6, TCF3-PBX1 or TCF3-TFPT, characteristic of acute lymphocytic leukemia (ALL); BCL11B-TLX3, IL2-TNFRFS17, NUP214-ABL1, NUP98-CCDC28A, TAL1-STIL, or ETV6-ABL2, characteristic of T-cell acute lymphocytic leukemia (T-ALL); ATIC-ALK, KIAA1618-ALK, MSN-ALK, MYH9-ALK, NPM1-ALK, TGF-ALK or TPM3-ALK, characteristic of anaplastic large cell lymphoma (ALCL); BCR-ABL1, BCR-JAK2, ETV6-EVI1, ETV6-MN1 or ETV6-TCBA1, characteristic of chronic myelogenous leukemia (CML); CBFB-MYH11, CHIC2-ETV6, ETV6-ABL1, ETV6-ABL2, ETV6-ARNT, ETV6-CDX2, ETV6-HLXB9, ETV6-PER1, MEF2D-DAZAP1, AML-AFF1, MLL-ARHGAP26, MLL-ARHGEF12, MLL-CASC5, MLL-CBL, MLL-CREBBP, MLL-DAB21P, MLL-ELL, MLL-EP300, MLL-EPS15, MLL-FNBP1, MLL-FOXO3A, MLL-GMPS, MLL-GPHN, MLL-MLLT1, MLL-MLLT11, MLL-MLLT3, MLL-MLLT6, MLL-MYO1F, MLL-PICALM, MLL-SEPT2, MLL-SEPT6, MLL-SORBS2, MYST3-SORBS2, MYST-CREBBP, NPM1-MLF1, NUP98-HOXA13, PRDM16-EVI1, RABEP1-PDGFRB, RUNX1-EVI1, RUNX1-MDS1, RUNX1-RPL22, RUNX1-RUNX1T1, RUNX1-SH3D19, RUNX1-USP42, RUNX1-YTHDF2, RUNX1-ZNF687, or TAF15-ZNF-384, characteristic of AML; CCND1-FSTL3, characteristic of chronic lymphocytic leukemia (CLL); BCL3-MYC, MYC-BTG1, BCL7A-MYC, BRWD3-ARHGAP20 or BTG1-MYC, characteristic of B-cell chronic lymphocytic leukemia (B-CLL); CITTA-BCL6, CLTC-ALK, IL21R-BCL6, PIM1-BCL6, TFCR-BCL6, IKZF1-BCL6 or SEC31A-ALK, characteristic of diffuse large B-cell lymphomas (DLBCL); FLIP1-PDGFRA, FLT3-ETV6, KIAA1509-PDGFRA, PDE4DIP-PDGFRB, NIN-PDGFRB, TP53BP1-PDGFRB, or TPM3-PDGFRB, characteristic of hyper eosinophilia/chronic eosinophilia; IGH-MYC or LCP1-BCL6, characteristic of Burkitt's lymphoma. The exosome can be derived from blood cancer cells.

Also provided herein is an isolated exosome comprising one or more gene fusions as disclosed herein, such as listed in FIG. 59. A composition comprising the isolated exosome is also provided. Accordingly, in some embodiments, the composition comprises a population of exosomes comprising one or more gene fusions, such as listed in FIG. 59. The composition can comprise a substantially enriched population of exosomes, wherein the population of exosomes is substantially homogeneous for exosomes comprising one or more gene fusions, such as listed in FIG. 59.

Also provided herein is a detection system for detecting one or more gene fusions, such as gene fusions listed in FIG. 59. For example, a detection system can comprise one or more probes to detect one or more gene fusions listed in FIG. 59. Detection of the one or more gene fusions can be used to charcaterize a cancer.

Gene-Associated MiRNA Biomarkers

The one or more biomarkers assessed can also include one or more genes selected from the group consisting of PFKFB3, RHAMM (HMMR), cDNA F1142103, ASPM, CENPF, NCAPG, Androgen Receptor, EGFR, HSP90, SPARC, DNMT3B, GART, MGMT, SSTR3, and TOP2B. The microRNA that interacts with the one or more genes can also be a biomarker (see for example, FIG. 60). Furthermore, the one or more biomarkers can be used to characterize prostate cancer.

Also provided herein is an isolated exosome comprising one or more one or more biomarkers consisting of PFKFB3, RHAMM (HMMR), cDNA F1142103, ASPM, CENPF, NCAPG, Androgen Receptor, EGFR, HSP90, SPARC, DNMT3B, GART, MGMT, SSTR3, and TOP2B; or the microRNA that interacts with the one or more genes (see for example, FIG. 60). Also provided is a composition comprising the isolated exosome. Accordingly, in some embodiments, the composition comprises a population of exosomes comprising one or more biomarkers consisting of PFKFB3, RHAMM (HMMR), cDNA F1142103, ASPM, CENPF, NCAPG, Androgen Receptor, EGFR, HSP90, SPARC, DNMT3B, GART, MGMT, SSTR3, and TOP2B; or the microRNA that interacts with the one or more genes, such as listed in FIG. 60. The composition can comprise a substantially enriched population of exosomes, wherein the population of exosomes is substantially homogeneous for exosomes comprising one or more biomarkers consisting of PFKFB3, RHAMM (HMMR), cDNA F1142103, ASPM, CENPF, NCAPG, Androgen Receptor, EGFR, HSP90, SPARC, DNMT3B, GART, MGMT, SSTR3, and TOP2B; or the microRNA that interacts with the one or more genes, such as listed in FIG. 60.

One or more prostate cancer specific biomarkers, such as listed in FIG. 60 can also be detected by one or more systems disclosed herein. For example, a detection system can comprise one or more probes to detect one or more prostate cancer specific biomarkers, such as listed in FIG. 60, of one or more exosomes of a biological sample.

The miRNA that interacts with PFKFB3 can be miR-513a-3p, miR-128, miR-488, miR-539, miR-658, miR-524-5p, miR-1258, miR-150, miR-216b, miR-377, miR-135a, miR-26a, miR-548a-5p, miR-26b, miR-520d-5p, miR-224, miR-1297, miR-1197, miR-182, miR-452, miR-509-3-5p, miR-548m, miR-625, miR-509-5p, miR-1266, miR-135b, miR-190b, miR-496, miR-616, miR-621, miR-650, miR-105, miR-19a, miR-346, miR-620, miR-637, miR-651, miR-1283, miR-590-3p, miR-942, miR-1185, miR-577, miR-602, miR-1305, miR-220c, miR-1270, miR-1282, miR-432, miR-491-5p, miR-548n, miR-765, miR-768-3p or miR-924, and can be used as a biomarker.

Also provided herein is an isolated exosome comprising one or more one or more miRNA that interacts with PFKFB3. Also provided is a composition comprising the isolated exosome. Accordingly, in some embodiments, the composition comprises a population of exosomes comprising one or more biomarkers consisting of miRNA that interacts with PFKFB3. The composition can comprise a substantially enriched population of exosomes, wherein the population of exosomes is substantially homogeneous for exosomes comprising one or more miRNA that interacts with PFKFB3. Furthermore, the one or more miRNA that interacts with PFKFB3 can also be detected by one or more systems disclosed herein. For example, a detection system can comprise one or more probes to detect one or more one or more miRNA that interacts with PFKFB3 of one or more exosomes of a biological sample.

The miRNA that interacts with RHAMM can be miR-936, miR-656, miR-105, miR-361-5p, miR-194, miR-374a, miR-590-3p, miR-186, miR-769-5p, miR-892a, miR-380, miR-875-3p, miR-208a, miR-208b, miR-586, miR-125a-3p, miR-630, miR-374b, miR-411, miR-629, miR-1286, miR-1185, miR-16, miR-200b, miR-671-5p, miR-95, miR-421, miR-496, miR-633, miR-1243, miR-127-5p, miR-143, miR-15b, miR-200c, miR-24 or miR-34c-3p.

Also provided herein is an isolated exosome comprising one or more one or more miRNA that interacts with RHAMM. Also provided is a composition comprising the isolated exosome. Accordingly, in some embodiments, the composition comprises a population of exosomes comprising one or more biomarkers consisting of miRNA that interacts with RHAMM. The composition can comprise a substantially enriched population of exosomes, wherein the population of exosomes is substantially homogeneous for exosomes comprising one or more miRNA that interacts with RHAMM. Furthermore, the one or more miRNA that interacts with RHAMMcan also be detected by one or more systems disclosed herein. For example, a detection system can comprise one or more probes to detect one or more one or more miRNA that interacts with RHAMM of one or more exosomes of a biological sample.

The miRNA that interacts with CENPF can be miR-30c, miR-30b, miR-190, miR-508-3p, miR-384, miR-512-5p, miR-548p, miR-297, miR-520f, miR-376a, miR-1184, miR-577, miR-708, miR-205, miR-376b, miR-520g, miR-520h, miR-519d, miR-596, miR-768-3p, miR-340, miR-620, miR-539, miR-567, miR-671-5p, miR-1183, miR-129-3p, miR-636, miR-106a, miR-1301, miR-17, miR-20a, miR-570, miR-656, miR-1263, miR-1324, miR-142-5p, miR-28-5p, miR-302b, miR-452, miR-520d-3p, miR-548o, miR-892b, miR-302d, miR-875-3p, miR-106b, miR-1266, miR-1323, miR-20b, miR-221, miR-520e, miR-664, miR-920, miR-922, miR-93, miR-1228, miR-1271, miR-30e, miR-483-3p, miR-509-3-5p, miR-515-3p, miR-519e, miR-520b, miR-520c-3p or miR-582-3p.

Also provided herein is an isolated exosome comprising one or more one or more miRNA that interacts with CENPF. Also provided is a composition comprising the isolated exosome. Accordingly, in some embodiments, the composition comprises a population of exosomes comprising one or more biomarkers consisting of miRNA that interacts with CENPF. The composition can comprise a substantially enriched population of exosomes, wherein the population of exosomes is substantially homogeneous for exosomes comprising one or more miRNA that interacts with CENPF. Furthermore, the one or more miRNA that interacts with CENPF can also be detected by one or more systems disclosed herein. For example, a detection system can comprise one or more probes to detect one or more one or more miRNA that interacts with CENPF of one or more exosomes of a biological sample.

The miRNA that interacts with NCAPG can be miR-876-5p, miR-1260, miR-1246, miR-548c-3p, miR-1224-3p, miR-619, miR-605, miR-490-5p, miR-186, miR-448, miR-129-5p, miR-188-3p, miR-516b, miR-342-3p, miR-1270, miR-548k, miR-654-3p, miR-1290, miR-656, miR-34b, miR-520g, miR-123I, miR-1289, miR-1229, miR-23a, miR-23b, miR-616 or miR-620.

Also provided herein is an isolated exosome comprising one or more one or more miRNA that interacts with NCAPG. Also provided is a composition comprising the isolated exosome. Accordingly, in some embodiments, the composition comprises a population of exosomes comprising one or more biomarkers consisting of miRNA that interacts with NCAPG. The composition can comprise a substantially enriched population of exosomes, wherein the population of exosomes is substantially homogeneous for exosomes comprising one or more miRNA that interacts with NCAPG. Furthermore, the one or more miRNA that interacts with NCAPG can also be detected by one or more systems disclosed herein. For example, a detection system can comprise one or more probes to detect one or more one or more miRNA that interacts with NCAPG of one or more exosomes of a biological sample.

, The miRNA that interacts with Androgen Receptor can be miR-124a, miR-130a, miR-130b, miR-143, miR-149, miR-194, miR-29b, miR-29c, miR-301, miR-30a-5p, miR-30d, miR-30e-5p, miR-337, miR-342, miR-368, miR-488, miR-493-5p, miR-506, miR-512-5p, miR-644, miR-768-5p or miR-801.

The miRNA that interacts with EGFR can be miR-105, miR-128a, miR-128b, miR-140, miR-141, miR-146a, miR-146b, miR-27a, miR-27b, miR-302a, miR-302d, miR-370, miR-548c, miR-574, miR-587 or miR-7.

Also provided herein is an isolated exosome comprising one or more one or more miRNA that interacts with AR. Also provided is a composition comprising the isolated exosome. Accordingly, in some embodiments, the composition comprises a population of exosomes comprising one or more biomarkers consisting of miRNA that interacts with AR. The composition can comprise a substantially enriched population of exosomes, wherein the population of exosomes is substantially homogeneous for exosomes comprising one or more miRNA that interacts with AR. Furthermore, the one or more miRNA that interacts with AR can also be detected by one or more systems disclosed herein. For example, a detection system can comprise one or more probes to detect one or more one or more miRNA that interacts with AR of one or more exosomes of a biological sample.

The miRNA that interacts with HSP90 can be miR-1, miR-513a-3p, miR-548d-3p, miR-642, miR-206, miR-450b-3p, miR-152, miR-148a, miR-148b, miR-188-3p, miR-23a, miR-23b, miR-578, miR-653, miR-1206, miR-192, miR-215, miR-181b, miR-181d, miR-223, miR-613, miR-769-3p, miR-99a, miR-100, miR-454, miR-548n, miR-640, miR-99b, miR-150, miR-181a, miR-181c, miR-522, miR-624, miR-130a, miR-130b, miR-146, miR-148a, miR-148b, miR-152, miR-181a, miR-181b, miR-181c, miR-204, miR-206, miR-211, miR-212, miR-215, miR-223, miR-23a, miR-23b, miR-301, miR-31, miR-325, miR-363, miR-566, miR-9 or miR-99b.

Also provided herein is an isolated exosome comprising one or more one or more miRNA that interacts with HSP90. Also provided is a composition comprising the isolated exosome. Accordingly, in some embodiments, the composition comprises a population of exosomes comprising one or more biomarkers consisting of miRNA that interacts with HSP90. The composition can comprise a substantially enriched population of exosomes, wherein the population of exosomes is substantially homogeneous for exosomes comprising one or more miRNA that interacts with HSP90. Furthermore, the one or more miRNA that interacts with HSP90 can also be detected by one or more systems disclosed herein. For example, a detection system can comprise one or more probes to detect one or more one or more miRNA that interacts with HSP90 of one or more exosomes of a biological sample.

The miRNA that interacts with SPARC can be miR-768-5p, miR-203, miR-196a, miR-569, miR-187, miR-641, miR-1275, miR-432, miR-622, miR-296-3p, miR-646, miR-196b, miR-499-5p, miR-590-5p, miR-495, miR-625, miR-1244, miR-512-5p, miR-1206, miR-1303, miR-186, miR-302d, miR-494, miR-562, miR-573, miR-10a, miR-203, miR-204, miR-211, miR-29, miR-29b, miR-29c, miR-339, miR-433, miR-452, miR-515-5p, miR-517a, miR-517b, miR-517c, miR-592 or miR-96.

Also provided herein is an isolated exosome comprising one or more one or more miRNA that interacts with SPARC. Also provided is a composition comprising the isolated exosome. Accordingly, in some embodiments, the composition comprises a population of exosomes comprising one or more biomarkers consisting of miRNA that interacts with SPARC. The composition can comprise a substantially enriched population of exosomes, wherein the population of exosomes is substantially homogeneous for exosomes comprising one or more miRNA that interacts with SPARC. Furthermore, the one or more miRNA that interacts with SPARC can also be detected by one or more systems disclosed herein. For example, a detection system can comprise one or more probes to detect one or more one or more miRNA that interacts with SPARC of one or more exosomes of a biological sample.

The miRNA that interacts with DNMT3B can be miR-618, miR-1253, miR-765, miR-561, miR-330-5p, miR-326, miR-188, miR-203, miR-221, miR-222, miR-26a, miR-26b, miR-29a, miR-29b, miR-29c, miR-370, miR-379, miR-429, miR-519e, miR-598, miR-618 or miR-635.

Also provided herein is an isolated exosome comprising one or more one or more miRNA that interacts with DNMT3B. Also provided is a composition comprising the isolated exosome. Accordingly, in some embodiments, the composition comprises a population of exosomes comprising one or more biomarkers consisting of miRNA that interacts with DNMT3B. The composition can comprise a substantially enriched population of exosomes, wherein the population of exosomes is substantially homogeneous for exosomes comprising one or more miRNA that interacts with DNMT3B. Furthermore, the one or more miRNA that interacts with DNMT3B can also be detected by one or more systems disclosed herein. For example, a detection system can comprise one or more probes to detect one or more one or more miRNA that interacts with DNMT3B of one or more exosomes of a biological sample.

The miRNA that interacts with GARTcan be miR-101, miR-141, miR-144, miR-182, miR-189, miR-199a, miR-199b, miR-200a, miR-200b, miR-202, miR-203, miR-223, miR-329, miR-383, miR-429, miR-433, miR-485-5p, miR-493-5p, miR-499, miR-519a, miR-519b, miR-519c, miR-569, miR-591, miR-607, miR-627, miR-635, miR-636 or miR-659.

Also provided herein is an isolated exosome comprising one or more one or more miRNA that interacts with GART. Also provided is a composition comprising the isolated exosome. Accordingly, in some embodiments, the composition comprises a population of exosomes comprising one or more biomarkers consisting of miRNA that interacts with GART. The composition can comprise a substantially enriched population of exosomes, wherein the population of exosomes is substantially homogeneous for exosomes comprising one or more miRNA that interacts with GART. Furthermore, the one or more miRNA that interacts with GART can also be detected by one or more systems disclosed herein. For example, a detection system can comprise one or more probes to detect one or more one or more miRNA that interacts with GART of one or more exosomes of a biological sample.

The miRNA that interacts with MGMT can be miR-122a, miR-142-3p, miR-17-3p, miR-181a, miR-181b, miR-181c, miR-181d, miR-199b, miR-200a, miR-217, miR-302b, miR-32, miR-324-3p, miR-34a, miR-371, miR-425-5p, miR-496, miR-514, miR-515-3p, miR-516-3p, miR-574, miR-597, miR-603, miR-653, miR-655, miR-92, miR-92b or miR-99a.

Also provided herein is an isolated exosome comprising one or more one or more miRNA that interacts with MGMT. Also provided is a composition comprising the isolated exosome. Accordingly, in some embodiments, the composition comprises a population of exosomes comprising one or more biomarkers consisting of miRNA that interacts with MGMT. The composition can comprise a substantially enriched population of exosomes, wherein the population of exosomes is substantially homogeneous for exosomes comprising one or more miRNA that interacts with MGMT. Furthermore, the one or more miRNA that interacts with MGMT can also be detected by one or more systems disclosed herein. For example, a detection system can comprise one or more probes to detect one or more one or more miRNA that interacts with MGMT of one or more exosomes of a biological sample.

The miRNA that interacts with SSTR3 can be miR-125a, miR-125b, miR-133a, miR-133b, miR-136, miR-150, miR-21, miR-380-5p, miR-504, miR-550, miR-671, miR-766 or miR-767-3p.

Also provided herein is an isolated exosome comprising one or more one or more miRNA that interacts with SSTR3. Also provided is a composition comprising the isolated exosome. Accordingly, in some embodiments, the composition comprises a population of exosomes comprising one or more biomarkers consisting of miRNA that interacts with SSTR3. The composition can comprise a substantially enriched population of exosomes, wherein the population of exosomes is substantially homogeneous for exosomes comprising one or more miRNA that interacts with SSTR3. Furthermore, the one or more miRNA that interacts with SSTR3 can also be detected by one or more systems disclosed herein. For example, a detection system can comprise one or more probes to detect one or more one or more miRNA that interacts with SSTR3 of one or more exosomes of a biological sample.

The miRNA that interacts with TOP2B can be miR-548f, miR-548a-3p, miR-548g, miR-513a-3p, miR-548c-3p, miR-101, miR-653, miR-548d-3p, miR-575, miR-297, miR-576-3p, miR-548b-3p, miR-624, miR-548n, miR-758, miR-1253, miR-1324, miR-23b, miR-320a, miR-320b, miR-1183, miR-1244, miR-23a, miR-451, miR-568, miR-1276, miR-548e, miR-590-3p, miR-1, miR-101, miR-126, miR-129, miR-136, miR-140, miR-141, miR-144, miR-147, miR-149, miR-18, miR-181b, miR-181c, miR-182, miR-184, miR-186, miR-189, miR-191, miR-19a, miR-19b, miR-200a, miR-206, miR-210, miR-218, miR-223, miR-23a, miR-23b, miR-24, miR-27a, miR-302, miR-30a, miR-31, miR-320, miR-323, miR-362, miR-374, miR-383, miR-409-3p, miR-451, miR-489, miR-493-3p, miR-514, miR-542-3p, miR-544, miR-548a, miR-548b, miR-548c, miR-548d, miR-559, miR-568, miR-575, miR-579, miR-585, miR-591, miR-598, miR-613, miR-649, miR-651, miR-758, miR-768-3p or miR-9.

Also provided herein is an isolated exosome comprising one or more one or more miRNA that interacts with TOP2B. Also provided is a composition comprising the isolated exosome. Accordingly, in some embodiments, the composition comprises a population of exosomes comprising one or more biomarkers consisting of miRNA that interacts with TOP2B. The composition can comprise a substantially enriched population of exosomes, wherein the population of exosomes is substantially homogeneous for exosomes comprising one or more miRNA that interacts with TOP2B. Furthermore, the one or more miRNA that interacts with TOP2B can also be detected by one or more systems disclosed herein. For example, a detection system can comprise one or more probes to detect one or more one or more miRNA that interacts with TOP2B of one or more exosomes of a biological sample.

### Bio-Signatures: Biomarker Detection

Bio-signatures can be detected qualitatively or quantitatively. Exosome levels may be characterized as described above. Analysis of exosomes can comprise detecting the level of exosomes in combination with determining the biomarkers of the exosomes. Determining the level or amount of exosome can be performed in conjunction with determining the biomarkers of the exosome. Alternatively, determining the amount of exosome may be performed prior to or subsequent to determining the biomarkers of the exosomes. Methods for analyzing biomarkers of tissues or cells can be used to analyze the biomarkers associated with or contained in exosomes.

For example, biomarkers can be detected by microarray analysis, PCR (including PCR-based methods such as RT-PCR, qPCR and the like), hybridization with allele-specific probes, enzymatic mutation detection, ligation chain reaction (LCR), oligonucleotide ligation assay (OLA), flow-cytometric heteroduplex analysis, chemical cleavage of mismatches, mass spectrometry, nucleic acid sequencing, single strand conformation polymorphism (SSCP), denaturing gradient gel electrophoresis (DGGE), temperature gradient gel electrophoresis (TGGE), restriction fragment polymorphisms, serial analysis of gene expression (SAGE), or any combinations thereof. The biomarker, such as a nucleic acid, can be amplified prior to detection. Biomarkers can also be detected by immunoblot, immunoprecipitation, ELISA, RIA, flow cytometry, or electron microscopy.

One method of detecting biomarkers can include purifying or isolating a heterogeneous exosome population from a biological sample, as described above, and performing a sandwich assay. An exosome in the population can be captured with a primary antibody, such as an antibody bound to a substrate, for example an array, well, or particle. The captured or bound exosome can be detected with a detection antibody. For example, the detection antibody can be for an antigen of the exosome. The detection antibody can be directly labeled and detected. Alternatively, an enzyme linked secondary antibody can react with the detection antibody. A detection reagent or detection substrate is added and the reaction can be detected, such as described in PCT Publication No. WO2009092386. The primary antibody can be an anti-Rab 5b antibody and the detection antibody anti-CD63 or anti-caveolin-1. Alternatively, the capture antibody can be an antibody to CD9, PSCA, TNFR, CD63, B7H3, MFG-E8, EpCam, Rab, CD81, STEAP, PCSA, PSMA, or 5T4. The detection antibody can be an antibody to CD63, CD9, CD81, B7H3, or EpCam.

In some embodiments, the capture agent binds or targets EpCam, and the one or more biomarkers detected on the exosome is CD9, CD63, or both CD9 and CD63. In other embodiments, the capture agent targets PCSA, and the one or more biomarkers detected on the captured exosome is B7H3, PSMA, or both B7H3 and PSMA. In yet other embodiments, the capture agent targets CD63 and the one or more biomarkers detected on the exosome is CD81, CD83, CD9, CD63, or any combination thereof. The different capture agent and biomarker combinations can be used to characterize a phenotype, such as prostate cancer or colon cancer. For example, capturing one or more exosomes can be performed with a capture agent targeting EpCam and detection of CD9 and CD63; a capture agent targeting PCSA and detection of B7H3 and PSMA; or a capture agent of CD63 and detection of CD81; can be used to characterize prostate cancer. A capture agent targeting CD63 and detection of CD63, or a capture agent targeting CD9 and detecting CD63, can be used to characterize colon cancer.

Other methods can include the use of a planar substrate such as an array (i.e., biochip or microarray), with immobilized molecules as capture agents, which can facilitate the detection of a particular bio-signature of exosomes. The arrays can be provided as part of a kit for assaying exosomes. Molecules that identify the biomarkers described above and shown in FIG. 3-60, as well as antigens in FIG. 1 can be included in a custom array for detection and diagnosis of diseases including presymtomatic diseases. Arrays comprising biomolecules that specifically identify selected biomarkers can be used to develop a database of information using data provided in the present specification. Additional biomolecules that identify bio-signatures which lead to improved cross-validated error rates in multivariate prediction models (e.g., logistic regression, discriminant analysis, or regression tree models) can be included in a custom array.

Customized array(s) provide an opportunity to study the biology of a disease, condition or syndrome and profile exosomes that are shed in defined physiological states. Standard p values of significance (0.05) can be chosen to exclude or include additional specific biomolecules on the microarray that identify particular biomarkers.

A planar array can generally contain addressable locations (e.g., pads, addresses, or micro-locations) of biomolecules in an array format. The size of the array will depend on the composition and end use of the array. Arrays containing from about 2 different molecules to many thousands can be made. Generally, the array can comprise from two to as many as 100,000 or more molecules, depending on the end use of the array and the method of manufacture. A microarray can generally comprise at least one biomolecule that identifies or captures a biomarker present in a bio-signature of specific cell-of-origin exosomes. In some embodiments, the compositions of the invention may not be in an array format; that is, for some embodiments, compositions comprising a single biomolecule may be made as well. In addition, in some arrays, multiple substrates may be used, either of different or identical compositions. Thus, for example, large planar arrays may comprise a plurality of smaller substrates.

An array of the present invention encompasses any means for detecting a biomarker. For example, microarrays can be biochips that provide high-density immobilized arrays of recognition molecules (e.g., antibodies), where biomarker binding is monitored indirectly (e.g., via fluorescence). In addition, an array can be of a format that involves the capture of proteins by biochemical or intermolecular interaction, coupled with direct detection by mass spectrometry (MS).

Arrays and microarrays that can be used to detect the biomarkers of a bio-signature of exosomes can be made according to the methods described in U.S. Pat. Nos. 6,329,209; 6,365,418; 6,406,921; 6,475,808; and 6,475,809, and U.S. patent application Ser. No. 10/884,269, each of which is herein incorporated by reference in its entirety. New arrays, to detect specific selections of sets of biomarkers described herein can also be made using the methods described in these patents. Furthermore, commercially available microarrays, such as for protein or nucleic acid detection can also be used, such as from Affymetrix (Santa Clara, Calif.), Illumina (San Diego, Calif.), Agilent (Santa Clara, Calif.), Exiqon (Denmark), or Invitrogen (Carlsbad, Calif.).

In many embodiments, immobilized molecules, or molecules to be immobilized, are proteins or peptides. One or more types of proteins may be immobilized on a surface. In certain embodiments, the proteins are immobilized using methods and materials that minimize the denaturing of the proteins, that minimize alterations in the activity of the proteins, or that minimize interactions between the protein and the surface on which they are immobilized.

Surfaces useful may be of any desired shape (form) and size. Non-limiting examples of surfaces include chips, continuous surfaces, curved surfaces, flexible surfaces, films, plates, sheets, tubes, or the like. Surfaces can have areas ranging from approximately a square micron to approximately 500 cm². The area, length, and width of surfaces according to the present invention may be varied according to the requirements of the assay to be performed. Considerations may include, for example, ease of handling, limitations of the material(s) of which the surface is formed, requirements of detection systems, requirements of deposition systems (e.g., arrayers), or the like.

In certain embodiments, it is desirable to employ a physical means for separating groups or arrays of binding islands or immobilized biomolecules: such physical separation facilitates exposure of different groups or arrays to different solutions of interest. Therefore, in certain embodiments, arrays are situated within microwell plates having any number of wells. In such embodiments, the bottoms of the wells may serve as surfaces for the formation of arrays, or arrays may be formed on other surfaces and then placed into wells. In certain embodiments, such as where a surface without wells is used, binding islands may be formed or molecules may be immobilized on a surface and a gasket having holes spatially arranged so that they correspond to the islands or biomolecules may be placed on the surface. Such a gasket is preferably liquid tight. A gasket may be placed on a surface at any time during the process of making the array and may be removed if separation of groups or arrays is no longer necessary.

The immobilized molecules can bind to exosomes present in a biological sample overlying the immobilized molecules. Alternatively, the immobilized molecules modify or are modified by molecules present in exosomes overlying the immobilized molecules.

Modifications or binding of molecules in solution or immobilized on an array may be detected using detection techniques known in the art. Examples of such techniques include immunological techniques such as competitive binding assays and sandwich assays; fluorescence detection using instruments such as confocal scanners, confocal microscopes, or CCD-based systems and techniques such as fluorescence, fluorescence polarization (FP), fluorescence resonant energy transfer (FRET), total internal reflection fluorescence (TIRF), fluorescence correlation spectroscopy (FCS); colorimetric/spectrometric techniques; surface plasmon resonance, by which changes in mass of materials adsorbed at surfaces may be measured; techniques using radioisotopes, including conventional radioisotope binding and scintillation proximity assays (SPA); mass spectroscopy, such as matrix-assisted laser desorption/ionization mass spectroscopy (MALDI) and MALDI-time of flight (TOF) mass spectroscopy; ellipsometry, which is an optical method of measuring thickness of protein films; quartz crystal microbalance (QCM), a very sensitive method for measuring mass of materials adsorbing to surfaces; scanning probe microscopies, such as AFM and SEM; and techniques such as electrochemical, impedance, acoustic, microwave, and IR/Raman detection. See, e.g., Mere L, et al., “Miniaturized FRET assays and microfluidics: key components for ultra-high-throughput screening,” Drug Discovery Today 4(8): 363-369 (1999), and references cited therein; Lakowicz J R, Principles of Fluorescence Spectroscopy, 2nd Edition, Plenum Press (1999), or Jain K K: Integrative Omics, Pharmacoproteomics, and Human Body Fluids. In: Thongboonkerd V, ed., ed. Proteomics of Human Body Fluids: Principles, Methods and Applications. Volume 1: Totowa, N.J.: Humana Press, 2007, each of which is herein incorporated by reference in its entirety.

Microarray technology can be combined with mass spectroscopy (MS) analysis and other tools. Electrospray interface to a mass spectrometer can be integrated with a capillary in microfluidics devices. For example, one commercially available system contains eTag reporters that are fluorescent labels with unique and well-defined electrophoretic mobilities; each label is coupled to biological or chemical probes via cleavable linkages. The distinct mobility address of each eTag reporter allows mixtures of these tags to be rapidly deconvoluted and quantitated by capillary electrophoresis. This system allows concurrent gene expression, protein expression, and protein function analyses from the same sample Jain K K: Integrative Omics, Pharmacoproteomics, and Human Body Fluids. In: Thongboonkerd V, ed., ed. Proteomics of Human Body Fluids: Principles, Methods and Applications. Volume 1: Totowa, N.J.: Humana Press, 2007, which is herein incorporated by reference in its entirety.

These biochips can include components for microfluidic or nanofluidic assays. Microfluidic devices can be used for isolating exosomes, such as described herein, in combination with analyzing the exosomes, such as determining bio-signatures. Such systems miniaturize and compartmentalize processes that allow for capturing of exosomes, detection of exosomal biomarkers, and other processes. The microfluidic devices can utilize detection reagents in at least one aspect of the system, and such detection reagents may be used to detect one or more biomarkers of exosomes. For example, the device can detect biomarkers on the isolated exosomes or bound exosomes. One or more biomarkers of a sample of isolated exosomes can be detected through the use of a microfluidic device. For example, various probes, antibodies, proteins, or other binding agents can be used to detect a biomarker. The detection agents may be immoblized in different compartments of the microfluidic device or be entered into a hybridization or detection reaction through various channels of the device.

An exosome in a microfluidic device may be lysed and the contents, such as proteins or nucleic acids, such as DNA or RNA (such as miRNA, mRNA) can be detected within a microfluidic device. The nucleic acid may be amplified prior to detection, or directly detected, within the microfluidic device. Thus microfluidic systems can also be used for multiplexing detection of various biomarkers.

Novel nanofabrication techniques are opening up the possibilities for biosensing applications that rely on fabrication of high-density, precision arrays, e.g., nucleotide-based chips and protein arrays otherwise know as heterogeneous nanoarrays. Nanofluidics allows a further reduction in the quantity of fluid analyte in a microchip to nanoliter levels, and the chips used here are referred to as nanochips. (See, e.g., Unger M et al., Biotechniques 1999; 27(5):1008-14, Kartalov E P et al., Biotechniques 2006; 40(1):85-90, each of which are herein incorporated by reference in their entireties.) Commercially available nanochips currently provide simple one step assays such as total cholesterol, total protein or glucose assays that can be run by combining sample and reagents, mixing and monitoring of the reaction. Gel-free analytical approaches based on liquid chromatography (LC) and nanoLC separations (Cutillas et al. Proteomics, 2005; 5:101-112 and Cutillas et al., Mol Cell Proteomics 2005; 4:1038-1051, each of which is herein incorporated by reference in its entirety) can be used in combination with the nanochips.

Arrays suitable for identifying a disease, condition or a syndrome or physiological status may be included in kits. Such kits may also include, as non-limiting examples, reagents useful for preparing molecules for immobilization onto binding islands or areas of an array, reagents useful for detecting binding of exosomes or exosomal components to immobilized molecules, and instructions for use.

Further provided herein is a rapid detection device that facilitates the detection of a particular bio-signature of exosomes in a biological sample. The device can integrate biological sample preparation with polymerase chain reaction (PCR) on a chip. The device can facilitate the detection of a particular bio-signature of exosomes in a biological sample, and an example is provided as described in Pipper et al., Angewandte Chemie, 47(21), p. 3900-3904 (2008), which is herein incorporated by reference in its entirety. The bio-signatures of the exosomes can be incorporated using micro-/nano-electrochemical system (MEMS/NEMS) sensors and oral fluid for diagnostic applications as described in Li et al., Adv Dent Res 18(1): 3-5 (2005), which is herein incorporated by reference in its entirety.

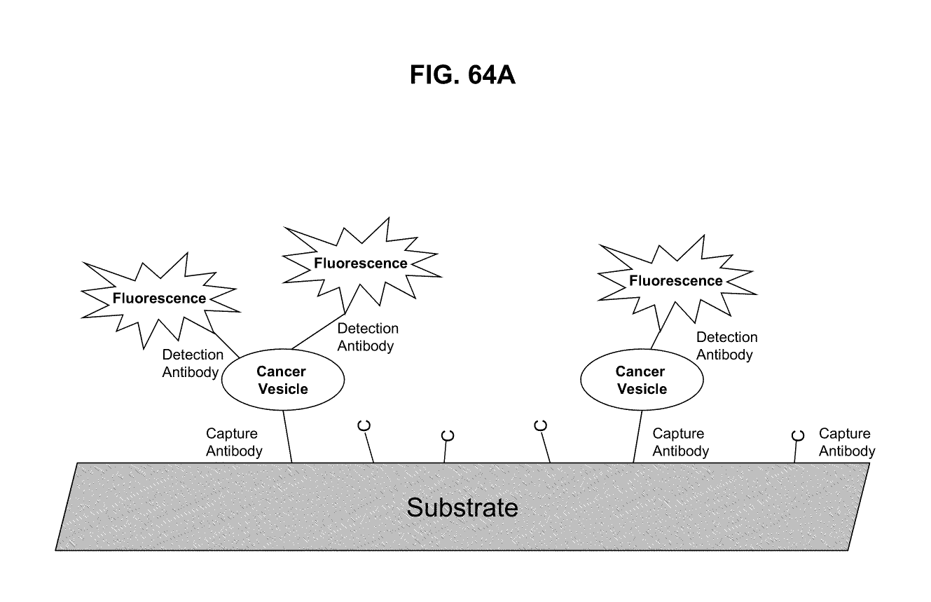

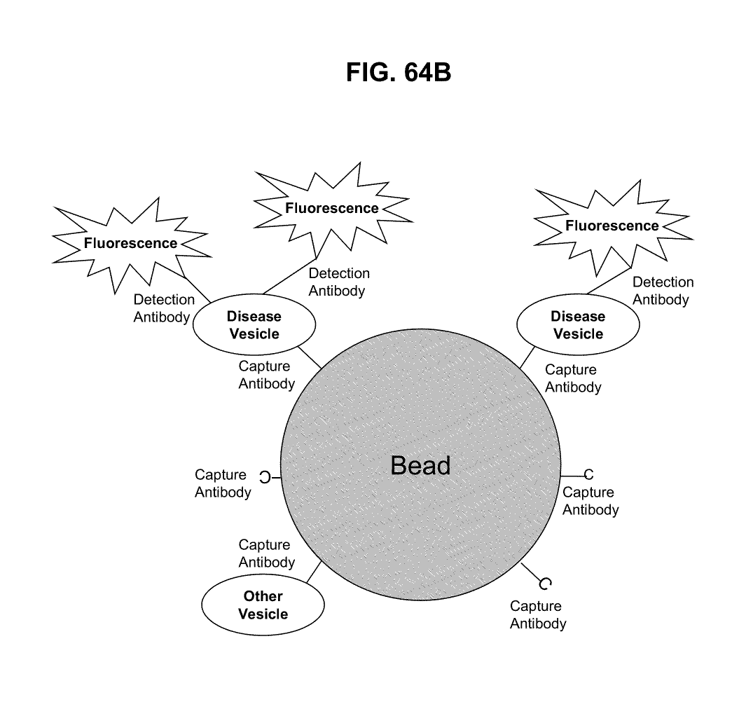

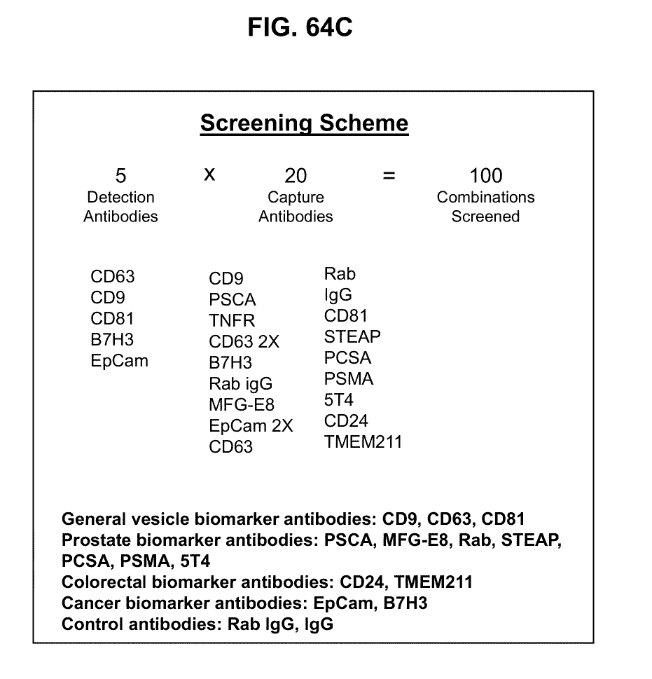

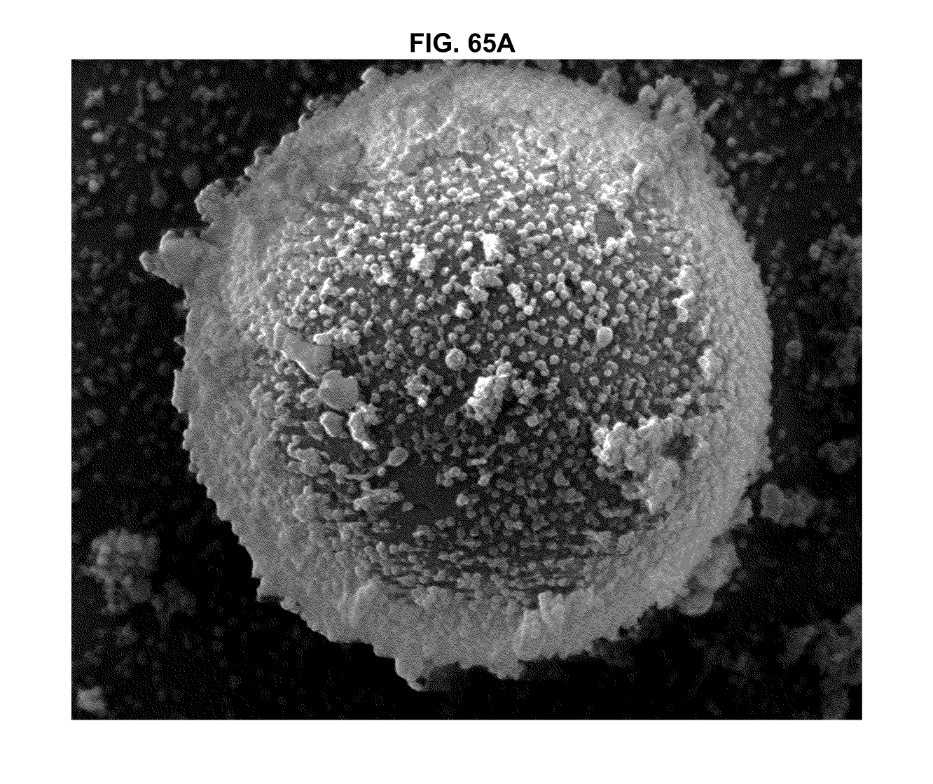

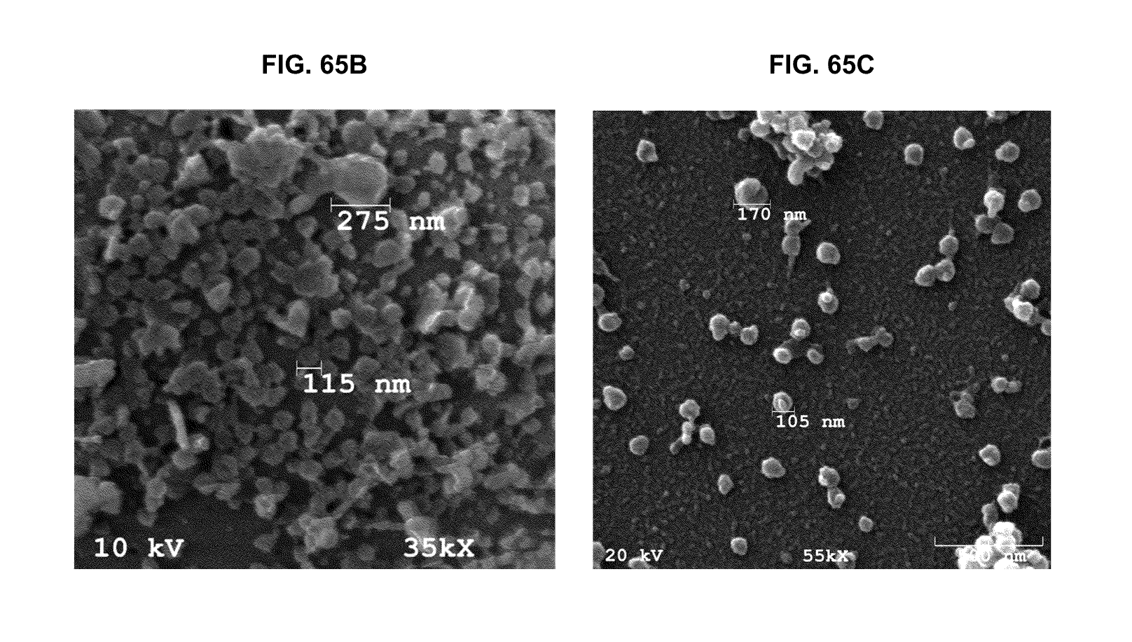

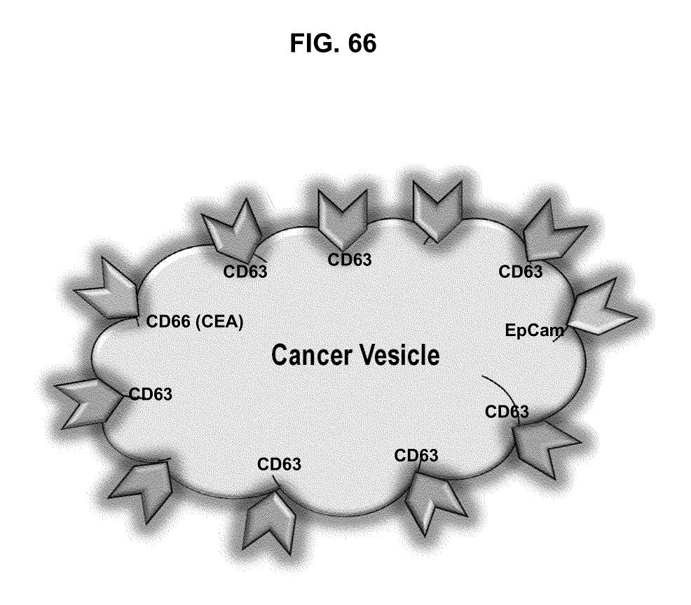

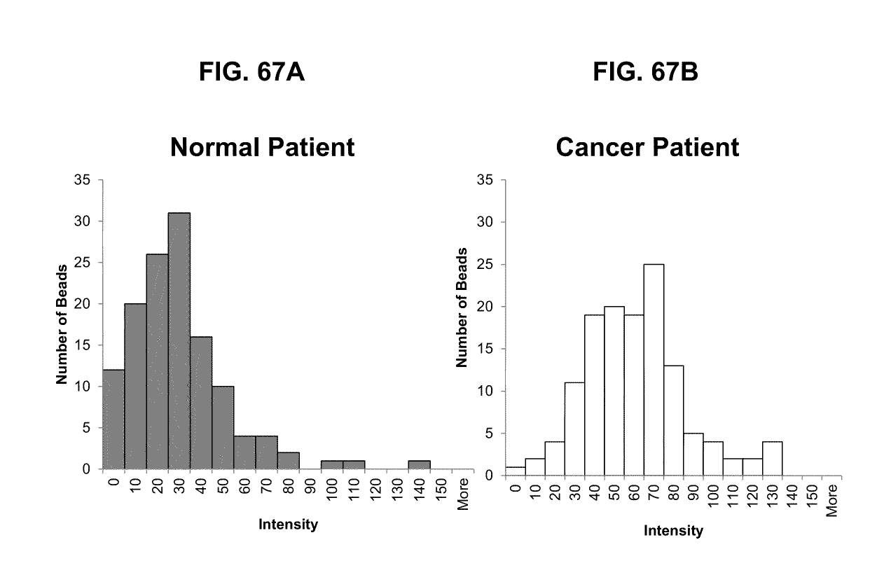

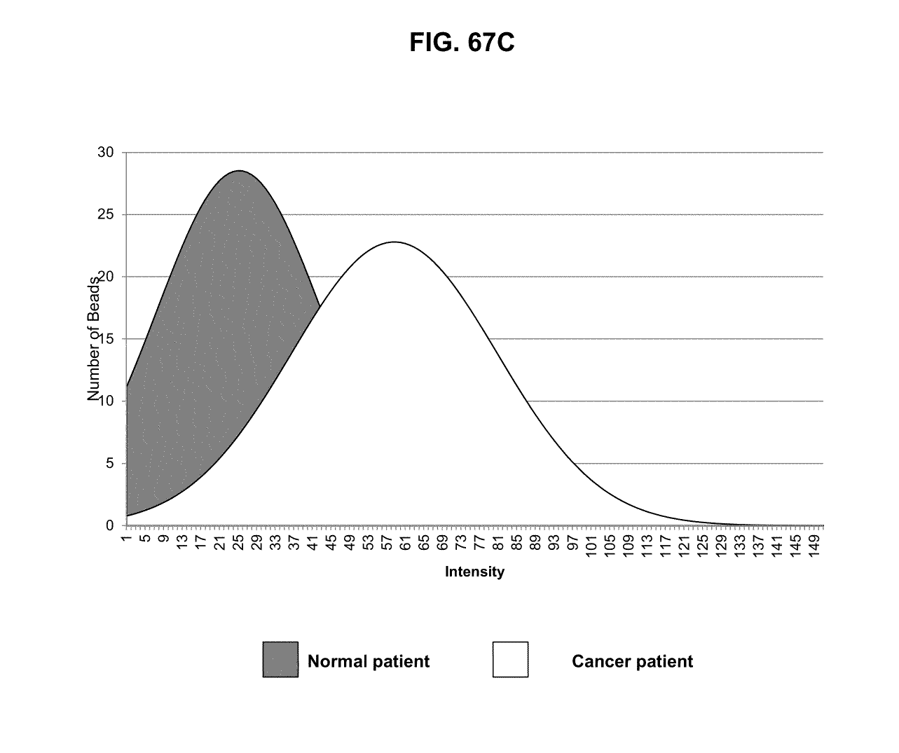

As an alternative to planar arrays, assays using particles, such as bead based assays as described herein, can be used in combination with flow cytometry. Multiparametric assays or other high throughput detection assays using bead coatings with cognate ligands and reporter molecules with specific activities consistent with high sensitivity automation can be used. In bead based assay systems, the binding agents, such as an antibody for exosomes, can be immobilized on addressable microspheres. Each binding agent for each individual binding assay is coupled to a distinct type of microsphere (i.e., microbead) and the assay reaction takes place on the surface of the microspheres, such as depicted in FIG. 64B. A binding agent for an exosome, such as a capture antibody is coupled to a bead. Dyed microspheres with discrete fluorescence intensities are loaded separately with their appropriate binding agent or capture probes. The different bead sets carrying different binding agents can be pooled as necessary to generate custom bead arrays. Bead arrays are then incubated with the sample in a single reaction vessel to perform the assay. Examples of microfluidic devices that may be used, or adapted for use with exosomes, include but are not limited to those described herein.

Product formation of the biomarker with their immobilized capture molecules or binding agents can be detected with a fluorescence based reporter system (see for example, FIGS. 64A-B). The biomarker can either be labeled directly by a fluorophore or detected by a second fluorescently labeled capture biomolecule. The signal intensities derived from captured biomarkers are measured in a flow cytometer. The flobw cytometer first identifies each microsphere by its individual color code. For example, distinct beads can be dyed with discrete fluorescence intensities such that each bead with a different intensity has a different binding agent. The beads can be labeled or dyed with at least 2 different labels or dyes. In some embodiments, the beads are labeled with at least 3, 4, 5, 6, 7, 8, 9, or 10 different labels. The beads with more than one label or dye can also have various ratios and combinations of the labels or dyes. The beads can be labeled or dyed externally or may have intrinsic fluorescence or signaling labels.

The amount of captured biomarkers on each individual bead can be measured by the second color fluorescence specific for the bound target. This allows multiplexed quantitation of multiple targets from a single sample within the same experiment. Sensitivity, reliability and accuracy are compared, or can be improved to standard microtiter ELISA procedures. An advantage of bead-based systems is the individual coupling of the capture biomolecule, or binding agent for an exosome, to distinct microspheres, which provides multiplexing. For example, as depicted in FIG. 64C, a combination of 5 different biomarkers to be detected (detected by antibodies to antigens such as CD63, CD9, CD81, B7H3, and EpCam) and 20 biomarkers for which to capture the exosome (using capture antibodies, such as antibodies to CD9, PSCA, TNFR, CD63, B7H3, MFG-E8, EpCam, Rab, CD81, STEAP, PCSA, PSMA, and 5T4) can result in 100 combinations to be detected. Thus, captured exosomes can be detected using detection agents, such as antibodies. The detection agents can be labeled directly or indirectly, such as described above.

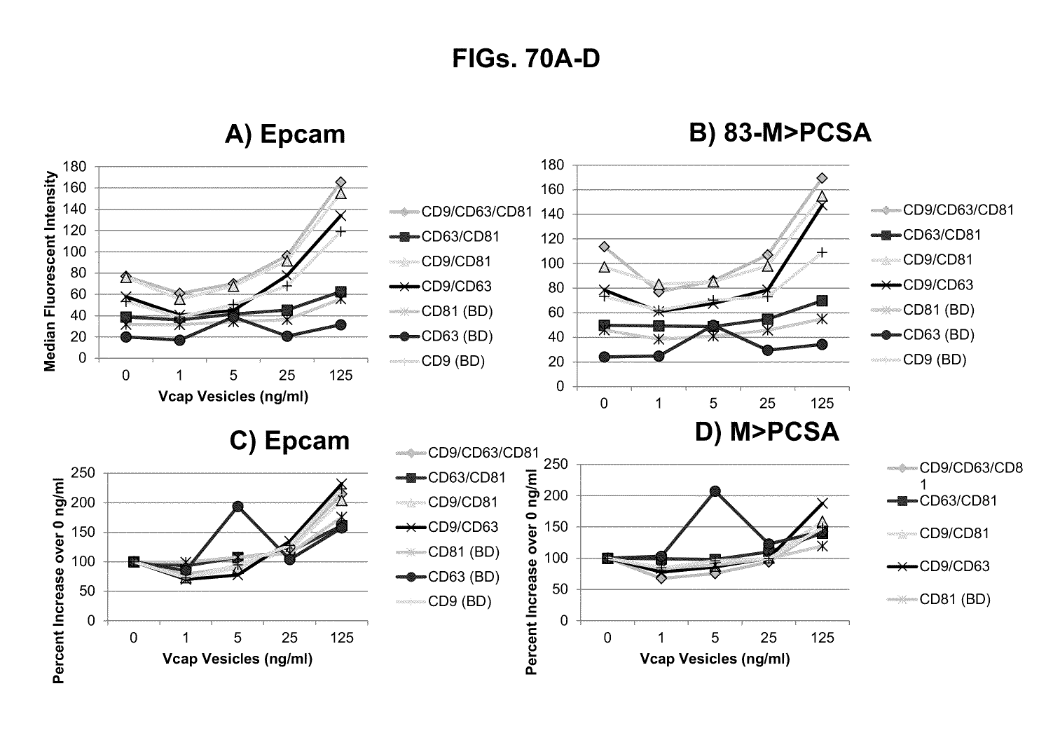

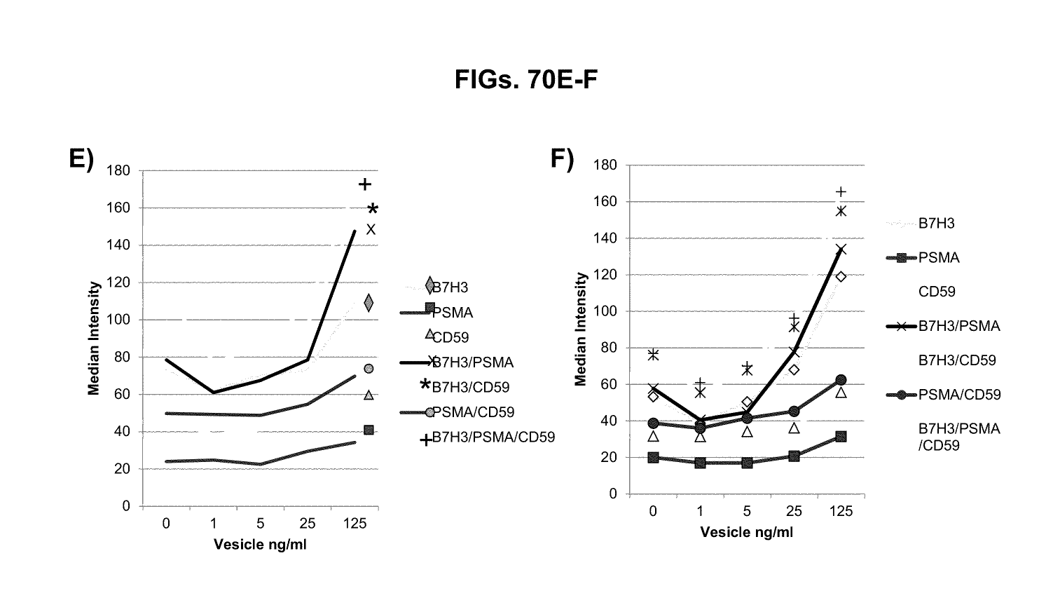

Multiplexing of at least 2, 3, 4, 5, 6, 7, 8, 9, 10, 11, 12, 13, 14, 15, 16, 17, 18, 19, 20, 25, 50, 75 or 100 different biomarkers may be performed. For example, an assay of a heterogeneous population of exosomes can be performed with a plurality of particles that are differentially labeled. There can be at least 2, 3, 4, 5, 6, 7, 8, 9, 10, 11, 12, 13, 14, 15, 16, 17, 18, 19, 20, 25, 50, 75 or 100 differentially labeled particles. The particles may be externally labeled, such as with a tag, or they may be intrinsically labeled. Each differentially labeled particle can be coupled to a capture agent, such as a binding agent, for an exosome, resulting in capture of an exosome. Biomarkers of the captured exosomes can then be detected by a plurality of binding agents. The binding agent can be directly labeled and thus, detected. Alternatively, the binding agent is labeled by a secondary agent. For example, the binding agent may be an antibody for a biomarker on the exosome. The binding agent is linked to biotin. A secondary agent comprises streptavidin linked to a reporter and can be added to detect the biomarker. In some embodiments, the captured exosomes are assayed for at least 2, 3, 4, 5, 6, 7, 8, 9, 10, 11, 12, 13, 14, 15, 16, 17, 18, 19, 20, 25, 50, 75 or 100 different biomarkers. For example, as depicted in FIG. 70, multiple detectors, i.e. detection of multiple biomarkers of a captured exosome, can increase the signal obtained, permitted increased sensitivity, specificity or both, and the use of smaller amounts of samples.

ELISA based methods, so sandwich assay can also be used to detect biomarkers on an exosome. A binding agent or capture agent can be bound to a well, for example an antibody to an exosomal antigen. Biomarkers on the captured exosome can be detected based on the methods described herein.

Peptide or protein biomakers can be analyzed by mass spectrometry or flow cytometry. Proteomic analysis of exosomes may also be carried out on exosomes by immunocytochemical staining, Western blotting, electrophoresis, chromatography or x-ray crystallography in accordance with procedures well known in the art. In other embodiments, the protein bio-signatures of exosomes may be analyzed using 2 D differential gel electrophoresis as described in, Chromy et al. J Proteome Res, 2004; 3:1120-1127, which is herein incorporated by reference in its entirety, or with liquid chromatography mass spectrometry as described in Zhang et al. Mol Cell Proteomics, 2005; 4:144-155, which is herein incorporated by reference in its entirety. Exosomes may be subjected to activity-based protein profiling described for example, in Berger et al., Am J Pharmacogenomics, 2004; 4:371-381, which is in incorporated by reference in its entirety. In other embodiments, exosomes may be profiled using nanospray liquid chromatography-tandem mass spectrometry as described in Pisitkun et al., Proc Natl Acad Sci USA, 2004; 101:13368-13373, which is herein incorporated by reference in its entirety. In another embodiment, the exosomes may be profiled using tandem mass spectrometry (MS) such as liquid chromatography/MS/MS (LC-MS/MS) using for example a LTQ and LTQ-FT ion trap mass spectrometer. Protein identification can be determined and relative quantitation can be assessed by comparing spectral counts as described in Smalley et al., J Proteome Res, 2008; 7:2088-2096, which is herein incorporated by reference in its entirety.

Protein expression of exosomes can also be identified, such as following the isolation of cell-of-origin specific exosomes, such exosomes can be resuspended in buffer, centrifuged at 100×g for example, for 3 minutes using a cytocentrifuge on adhesive slides in preparation for immunocytochemical staining. The cytospins can be air-dried overnight and stored at −80° C. until staining. Slides can then be fixed and blocked with serum-free blocking reagent. The slides can then be incubated with a specific antibody to detect the expression of a protein of interest. In some embodiments, the exosomes are not purified, isolated or concentrated prior to protein expression analysis.

Exosomes, such as isolated cell-of-origin specific exosomes can be characterized by analysis of metabolite markers or metabolites, which can also form a bio-signature for exosomes. Various metabolite-oriented approaches have been described such as metabolite target analyses, metabolite profiling, or metabolic fingerprinting, see for example, Denkert et al., Molecular Cancer 2008; 7: 4598-4617, Ellis et al., Analyst 2006; 8: 875-885, Kuhn et al., Clinical Cancer Research 2007; 24: 7401-7406, Fiehn O., Comp Funct Genomics 2001; 2:155-168, Fancy et al., Rapid Commun Mass Spectrom 20(15): 2271-80 (2006), Lindon et al., Pharm Res, 23(6): 1075-88 (2006), Holmes et al., Anal Chem. 2007 Apr. 1; 79(7):2629-40. Epub 2007 Feb. 27. Erratum in: Anal Chem. 2008 Aug. 1; 80(15):6142-3, Stanley et al., Anal Biochem. 2005 Aug. 15; 343(2): 195-202., Lehtimaki et al., J Biol Chem. 2003 Nov. 14; 278(46):45915-23, each of which is herein incorporated by reference in its entirety.

Peptides from exosomes can be analyzed by systems described in Jain K K: Integrative Omics, Pharmacoproteomics, and Human Body Fluids. In: Thongboonkerd V, ed., ed. Proteomics of Human Body Fluids: Principles, Methods and Applications. Volume 1: Totowa, N.J.: Humana Press, c2007., 2007, which is herein incorporated by reference in its entirety. This system can generate sensitive molecular fingerprints of proteins present in a body fluid as well as in exosomes. Commercial applications which include the use of chromatography/mass spectroscopy and reference libraries of all stable metabolites in the human body, for example Paradigm Genetic's Human Metabolome Project, may be used to determine the metabolite bio-signature of exosomes, such as isolated cell-of-origin specific exosomes. Other methods for analyzing a metabolic profile can include methods and devices described in U.S. Pat. No. 6,683,455 (Metabometrix), U.S. Patent Application Publication Nos. 20070003965 and 20070004044 (Biocrates Life Science), each of which is herein incorporated by reference in its entirety. Other proteomic profiling techniques are described in Kennedy, Toxicol Lett 120:379-384 (2001), Berven et al., Curr Pharm Biotechnol 7(3): 147-58 (2006), Conrads et al., Expert Rev Proteomics 2(5): 693-703, Decramer et al., World J Urol 25(5): 457-65 (2007), Decramer et al., Mol Cell Proteomics 7(10): 1850-62 (2008), Decramer et al., Contrib Nephrol, 160: 127-41 (2008), Diamandis, J Proteome Res 5(9): 2079-82 (2006), Immler et al., Proteomics 6(10): 2947-58 (2006), han et al., J Proteome Res 5(10): 2824-38 (2006), Kumar et al., Biomarkers 11(5): 385-405 (2006), Noble et al., Breast Cancer Res Treat 104(2): 191-6 (2007), Omenn, Dis Markers 20(3): 131-4 (2004), Powell et al., Expert Rev Proteomics 3(1): 63-74 (2006), Rai et al., Arch Pathol Lab Med, 126(12): 1518-26 (2002), Ramstrom et al., Proteomics, 3(2): 184-90 (2003), Tammen et al., Breast Cancer Res Treat, 79(1): 83-93 (2003), Theodorescu et al., Lancet Oncol, 7(3): 230-40 (2006), or Zurbig et al., Electrophoresis, 27(11): 2111-25 (2006).

For analysis of mRNAs, miRNAs or other small RNAs, the total RNA can be first isolated from exosomes using any other known methods for isolating nucleic acids such as methods described in U.S. Patent Application Publication No. 2008132694, which is herein incorporated by reference in its entirety. These include, but are not limited to, kits for performing membrane based RNA purification, which are commercially available. Generally, kits are available for the small-scale (30 mg or less) preparation of RNA from cells and tissues, for the medium scale (250 mg tissue) preparation of RNA from cells and tissues, and for the large scale (1 g maximum) preparation of RNA from cells and tissues. Other commercially available kits for effective isolation of small RNA-containing total RNA are available.

Alternatively, RNA can be isolated using the method described in U.S. Pat. No. 7,267,950, which is herein incorporated by reference in its entirety. U.S. Pat. No. 7,267,950 describes a method of extracting RNA from biological systems (cells, cell fragments, organelles, tissues, organs, or organisms) in which a solution containing RNA is contacted with a substrate to which RNA can bind and RNA is withdrawn from the substrate by applying negative pressure. Alternatively, RNA may be isolated using the method described in U.S. Patent Application No. 20050059024, which is herein incorporated by reference in its entirety, which describes the isolation of small RNA molecules. Other methods are described in U.S. Patent Application No. 20050208510, 20050277121, 20070238118, each of which is incorporated by reference in its entirety.

In one embodiment, mRNA expression analysis can be carried out on mRNAs from exosomes isolated from a sample. In some embodiments, the exosomes are cell-of-origin specific exosomes. Expression patterns generated from these exosomes can be indicative of a given disease state, disease stage, therapy related signature, or physiological condition. Once the total RNA has been isolated, cDNA can be synthesized and either qRT-PCR assays (e.g. Applied Biosystem's Taqman® assays) for specific mRNA targets can be performed according to manufacturer's protocol, or an expression microarray can be performed to look at highly multiplexed sets of expression markers in one experiment. Methods for establishing gene expression profiles include determining the amount of RNA that is produced by a gene that can code for a protein or peptide. This is accomplished by quantitative reverse transcriptase PCR (qRT-PCR), competitive RT-PCR, real time RT-PCR, differential display RT-PCR, Northern Blot analysis or other related tests. While it is possible to conduct these techniques using individual PCR reactions, it is also possible to amplify complementary DNA (cDNA) or complementary RNA (cRNA) produced from mRNA and analyze it via microarray.

The level of a miRNA product in a sample can be measured using any technique that is suitable for detecting mRNA expression levels in a biological sample, including but not limited to Northern blot analysis, RT-PCR, qRT-PCR, in situ hybridization or microarray analysis. For example, using gene specific primers and target cDNA, qRT-PCR enables sensitive and quantitative miRNA measurements of either a small number of target miRNAs (via singleplex and multiplex analysis) or the platform can be adopted to conduct high throughput measurements using 96-well or 384-well plate formats. See for example, Ross J S et al, Oncologist. 2008 May; 13(5):477-93, which is herein incorporated by reference in its entirety. A number of different array configurations and methods for microarray production are known to those of skill in the art and are described in U.S. patents such as: U.S. Pat. No. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; or 5,700,637; each of which is herein incorporated by reference in its entirety. Other methods of profiling miRNAs are described in Taylor et al., Gynecol Oncol. 2008 July; 110(1):13-21, Gilad et al, PLoS ONE. 2008 Sep. 5; 3(9):e3148, Lee et al., Annu Rev Pathol. 2008 Sep. 25 and Mitchell et al, Proc Natl Acad Sci USA. 2008 Jul. 29; 105(30): 10513-8, Shen R et al, BMC Genomics. 2004 Dec. 14; 5(1):94, Mina L et al, Breast Cancer Res Treat. 2007 June; 103(2):197-208, Zhang L et al, Proc Natl Acad Sci USA. 2008 May 13; 105(19):7004-9, Ross J S et al, Oncologist. 2008 May; 13(5):477-93, Schetter A J et al, JAMA. 2008 Jan. 30; 299(4):425-36, Staudt L M, N Engl J Med 2003; 348:1777-85, Mulligan G et al, Blood. 2007 Apr. 15; 109(8):3177-88. Epub 2006 Dec. 21, McLendon R et al, Nature. 2008 Oct. 23; 455(7216):1061-8, and U.S. Pat. Nos. 5,538,848, 5,723,591, 5,876,930, 6,030,787, 6,258,569, and 5,804,375, each of which is herein incorporated by reference.

Microarray technology allows for the measurement of the steady-state mRNA or miRNA levels of thousands of transcripts or miRNAs simultaneously thereby presenting a powerful tool for identifying effects such as the onset, arrest, or modulation of uncontrolled cell proliferation. Two microarray technologies, such as cDNA arrays and oligonucleotide arrays can be used. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA or miRNA, expressed in the sample cells. A large number of such techniques are available and useful. Methods for determining gene expression can be found in U.S. Pat. No. 6,271,002 to Linsley, et al.; U.S. Pat. No. 6,218,122 to Friend, et al.; U.S. Pat. No. 6,218,114 to Peck et al.; or U.S. Pat. No. 6,004,755 to Wang, et al., each of which is herein incorporated by reference in its entirety.

Analysis of the expression levels is conducted by comparing such intensities. This can be performed by generating a ratio matrix of the expression intensities of genes in a test sample versus those in a control sample. The control sample may be used as a reference, and different references to account for age, ethnicity and sex may be used. Different references can be used for different conditions or diseases, as well as different stages of disesaes or conditions, as well as for determining therapeutic efficacy.

For instance, the gene expression intensities of mRNA or miRNAs isolated from exosomes derived from a diseased tissue can be compared with the expression intensities generated from exosomes isolated from normal tissue of the same type (e.g., diseased breast tissue sample versus. normal breast tissue sample). A ratio of these expression intensities indicates the fold-change in gene expression between the test and control samples. Alternatively, if exosomes are not normally present in from normal tissues (e.g. breast) then absolute quantitation methods, as is known in the art, can be used to define the number of miRNA molecules present without the requirement of miRNA or mRNA isolated from exosomes derived from normal tissue.

Gene expression profiles can also be displayed in a number of ways. The most common method is to arrange raw fluorescence intensities or ratio matrix into a graphical dendogram where columns indicate test samples and rows indicate genes. The data is arranged so genes that have similar expression profiles are proximal to each other. The expression ratio for each gene is visualized as a color. For example, a ratio less than one (indicating down-regulation) may appear in the blue portion of the spectrum while a ratio greater than one (indicating up-regulation) may appear as a color in the red portion of the spectrum. Commercially available computer software programs are available to display such data.

mRNAs or miRNAs that are considered differentially expressed can be either over expressed or under expressed in patients with a disease relative to disease free individuals. Over and under expression are relative terms meaning that a detectable difference (beyond the contribution of noise in the system used to measure it) is found in the amount of expression of the mRNAs or miRNAs relative to some baseline. In this case, the baseline is the measured mRNA/miRNA expression of a non-diseased individual. The mRNA/miRNA of interest in the diseased cells can then be either over or under expressed relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis includes the determination of disease/status issues such as determining the likelihood of relapse or metastasis and therapy monitoring. In therapy monitoring, clinical judgments are made regarding the effect of a given course of therapy by comparing the expression of genes over time to determine whether the mRNA/miRNA expression profiles have changed or are changing to patterns more consistent with normal tissue.

Levels of over and under expression are distinguished based on fold changes of the intensity measurements of hybridized microarray probes. A 2× difference is preferred for making such distinctions or a p-value less than 0.05. That is, before an mRNA/miRNA is said to be differentially expressed in diseased/relapsing versus normal/non-relapsing cells, the diseased cell is found to yield at least 2 times more, or 2 times less intensity than the normal cells. The greater the fold difference, the more preferred is use of the gene as a diagnostic or prognostic tool. mRNA/miRNAs selected for the expression profiles of the instant invention have expression levels that result in the generation of a signal that is distinguishable from those of the normal or non-modulated genes by an amount that exceeds background using clinical laboratory instrumentation.

Statistical values can be used to confidently distinguish modulated from non-modulated mRNA/miRNA and noise. Statistical tests find the mRNA/miRNA most significantly different between diverse groups of samples. The Student's t-test is an example of a robust statistical test that can be used to find significant differences between two groups. The lower the p-value, the more compelling the evidence that the gene is showing a difference between the different groups. Nevertheless, since microarrays measure more than one mRNA/miRNA at a time, tens of thousands of statistical tests may be performed at one time. Because of this, one is unlikely to see small p-values just by chance and adjustments for this using a Sidak correction as well as a randomization/permutation experiment can be made. A p-value less than 0.05 by the t-test is evidence that the gene is significantly different. More compelling evidence is a p-value less then 0.05 after the Sidak correction is factored in. For a large number of samples in each group, a p-value less than 0.05 after the randomization/permutation test is the most compelling evidence of a significant difference.

In one embodiment, a method of generating a posterior probability score to enable diagnostic, prognostic, therapy-related, or physiological state specific bio-signature scores can be arrived at by obtaining mRNA or miRNA (biomarker) expression data from a statistically significant number of patient exosomes, such as cell-of-origin specific exosomes; applying linear discrimination analysis to the data to obtain selected biomarkers; and applying weighted expression levels to the selected biomarkers with discriminate function factor to obtain a prediction model that can be applied as a posterior probability score. Other analytical tools can also be used to answer the same question such as, logistic regression and neural network approaches.

For instance, the following can be used for linear discriminant analysis:

where,
I(pₛi_{d})=The log base 2 intensity of the probe set enclosed in parenthesis. d(cp)=The discriminant function for the disease positive class d(C_{N})=The discriminant function for the disease negative class
P_{(CP)}=The posterior p-value for the disease positive class
P_{(CN)}=The posterior p-value for the disease negative class

Numerous other well-known methods of pattern recognition are available. The following references provide some examples: Weighted Voting: Golub et al. (1999); Support Vector Machines: Su et al. (2001); and Ramaswamy et al. (2001); K-nearest Neighbors: Ramaswamy (2001); and Correlation Coefficients: van't Veer et al. (2002), all of which are herein incorporated by reference in their entireties.

Bio-signature portfolios, further described below, can be established such that the combination of biomarkers in the portfolio exhibit improved sensitivity and specificity relative to individual biomarkers or randomly selected combinations of biomarkers. In one embodiment, the sensitivity of the bio-signature portfolio can be reflected in the fold differences, for example, exhibited by a transcript's expression in the diseased state relative to the normal state. Specificity can be reflected in statistical measurements of the correlation of the signaling of transcript expression with the condition of interest. For example, standard deviation can be a used as such a measurement. In considering a group of biomarkers for inclusion in a bio-signature portfolio, a small standard deviation in expression measurements correlates with greater specificity. Other measurements of variation such as correlation coefficients can also be used in this capacity.

Another parameter that can be used to select mRNA/miRNA that generate a signal that is greater than that of the non-modulated mRNA/miRNA or noise is the use of a measurement of absolute signal difference. The signal generated by the modulated mRNA/miRNA expression is at least 20% different than those of the normal or non-modulated gene (on an absolute basis). It is even more preferred that such mRNA/miRNA produce expression patterns that are at least 30% different than those of normal or non-modulated mRNA/miRNA.

MiRNA can also be detected and measured by amplification from a biological sample and measured using methods described in U.S. Pat. No. 7,250,496, U.S. Application Publication Nos. 20070292878, 20070042380 or 20050222399 and references cited therein, each of which is herein incorporated by reference in its entirety.

Peptide nucleic acids (PNAs) which are a new class of synthetic nucleic acid analogs in which the phosphate-sugar polynucleotide backbone is replaced by a flexible pseudo-peptide polymer may be utilized in analysis of bio-signatures of exosomes. PNAs are capable of hybridizing with high affinity and specificity to complementary RNA and DNA sequences and are highly resistant to degradation by nucleases and proteinases. Peptide nucleic acids (PNAs) are an attractive new class of probes with applications in cytogenetics for the rapid in situ identification of human chromosomes and the detection of copy number variation (CNV). Multicolor peptide nucleic acid-fluorescence in situ hybridization (PNA-FISH) protocols have been described for the identification of several human CNV-related disorders and infectious diseases. PNAs can also be utilized as molecular diagnostic tools to non-invasively measure oncogene mRNAs with tumor targeted radionuclide-PNA-peptide chimeras. Methods of using PNAs are described further in Pellestor F et al, Curr Pharm Des. 2008; 14(24):2439-44, Tian X et al, Ann NY Acad Sci. 2005 November; 1059: 106-44, Paulasova P and Pellestor F, Annales de Génétique, 47 (2004) 349-358, Stender H. Expert Rev Mol Diagn. 2003 September; 3(5):649-55. Review, Vigneault et al., Nature Methods, 5(9), 777-779 (2008), each reference is herein incorporated by reference in its entirety. These methods can be used to screen the genetic materials isolated from exosomes. When applying these techniques to cell-of-origin specific exosomes they can be used to identify a given molecular signal that directly pertains to the cell of origin.

In addition, mutational analysis may be carried out for mRNAs and DNA that are identified from the exosomes. For mutational analysis of targets or biomarkers that are of RNA origin, the RNA (mRNA, miRNA or other) can be reverse transcribed into cDNA and subsequently sequenced or assayed for known SNPs (by Taqman SNP assays, for example), or single nucleotide mutations, as well as using sequencing to look for insertions or deletions to determine mutations present in the cell-of-origin. Muliplexed ligation dependent probe amplification (MLPA) could alternatively be used for the purpose of identifying CNV in small and specific areas of interest. For example, once the total RNA has been obtained from isolated colon cancer-specific exosomes, cDNA can be synthesized and primers specific for exons 2 and 3 of the KRAS gene can be used to amplify these two exons containing codons 12, 13 and 61 of the KRAS gene. The same primers used for PCR amplification can be used for Big Dye Terminator sequence analysis on the ABI 3730 to identify mutations in exons 2 and 3 of KRAS. Mutations in these codons are known to confer resistance to drugs such as Cetuximab and Panitumimab. Methods of conducting mutational analysis are described in Maheswaran S et al, Jul. 2, 2008 (10.1056/NEJMoa0800668) and Orita, M et al, PNAS 1989, (86): 2766-70, each of which is herein incorporated by reference in its entirety. Other methods of conducting mutational analysis can include miRNA sequencing. Applications for identifying and profiling miRNAs can be done by cloning techniques and the use of capillary DNA sequencing or “next-generation” sequencing technologies. The new sequencing technologies currently available allow the identification of low-abundance miRNAs or those exhibiting modest expression differences between samples, which may not be detected by hybridization-based methods. Such new sequencing technologies include the massively parallel signature sequencing (MPSS) methodology described in Nakano et al. 2006, Nucleic Acids Res. 2006; 34:D731 D735. doi: 10.1093/nar/gkj077, the Roche/454 platform described in Margulies et al. 2005, Nature. 2005; 437:376-380 or the Illumina sequencing platform described in Berezikov et al. Nat. Genet. 2006b; 38:1375-1377, each of which is incorporated by reference in its entirety.

Additional methods to determine bio-signatures include assaying biomarkers by allele-specific PCR which include specific primers to amplify and discriminate between two alleles of a gene simultaneously, single-strand conformation polymorphism (SSCP) which involves the electrophoretic separation of single-stranded nucleic acids based on subtle differences in sequence and DNA and RNA aptamers. DNA and RNA aptamers are short oligonucleotide sequences that can be selected from random pools based on their ability to bind a particular molecule with high affinity. Methods of using aptamers are described in Ulrich H et al, Comb Chem High Throughput Screen. 2006 September; 9(8):619-32, Ferreira C S et al, Anal Bioanal Chem. 2008 February; 390(4):1039-50, Ferreira C S et al, Tumour Biol. 2006; 27(6):289-301, each of which is herein incorporated by reference in its entirety.

Exosome biomarkers can also be detected using fluorescence in situ hybridization (FISH). Methods of using FISH to detect and localize specific DNA sequences, localize specific mRNAs within tissue samples or identify chromosomal abnormalities are described in Shaffer D R et al, Clin Cancer Res. 2007 Apr. 1; 13(7):2023-9, Cappuzo F et al, Journal of Thoracic Oncology, Volume 2, Number 5, May 2007, Moroni M et al, Lancet Oncol. 2005 May; 6(5):279-86, each of which is herein incorporated by reference in its entirety.

### Bio-Signature: Binding Agents

Bio-signatures of exosomes can comprise binding agents for exosomes. The binding agent can be DNA, RNA, aptamers, monoclonal antibodies, polyclonal antibodies, Fabs, Fab′, single chain antibodies, synthetic antibodies, aptamers (DNA/RNA), peptoids, zDNA, peptide nucleic acids (PNAs), locked nucleic acids (LNAs), lectins, synthetic or naturally occurring chemical compounds (including but not limited to drugs, labeling reagents).

Binding agents can used to isolate exosomes by binding to exosomal components, as described above. The binding agents can be used to detect the exosomes, such as for detecting cell-of-origin specific exosomes. A binding agent or multiple binding agents can themselves form a binding agent profile that provides a bio-signature for an exosome. One or more binding agents can be selected from FIG. 2. For example, if an exosome population is detected or isolated using two, three or four binding agents in a differential detection or isolation of an exosome from a heterogeneous population of exosomes, the particular binding agent profile for the exosome population provides a bio-signature for the particular exosome population.

As an illustrative example, an exosome for analysis for lung cancer can be detected with one or more binding agents including, but not limited to, SCLC specific aptamer HCA 12, SCLC specific aptamer HCCO3, SCLC specific aptamer HCH07, SCLC specific aptamer HCH01, A-p50 aptamer (NF-KB), Cetuximab, Panitumumab, Bevacizumab, L19 Ab, F16 Ab, anti-CD45 (anti-ICAM-1, aka UV3), or L2G7 Ab (anti-HGF), or any combination thereof.

An exosome for analysis for colon cancer can be detected with one or more binding agents including, but not limited to, angiopoietin 2 specific aptamer, beta-catenin aptamer, TCF1 aptamer, anti-Derlinl ab, anti-RAGE, mAbgb3.1, Galectin-3, Cetuximab, Panitumumab, Matuzumab, Bevacizumab, or Mac-2, or any combination thereof.

An exosome for analysis for adenoma versus colorectal cancer (CRC) can be detected with one or more binding agents including, but not limited to, Complement C3, histidine-rich glycoprotein, kininogen-1, or Galectin-3, or any combination thereof.

An exosome for analysis for adenoma with low grade hyperplasia versus adenoma with high grade hyperplasia can be detected with a binding agent such as, but not limited to, Galectin-3 or any combination of binding agents specific for this comparison.

An exosome for analysis for CRC versus normal state can be detected with one or more binding agents including, but not limited to, anti-ODC mAb, anti-CEA mAb, or Mac-2, or any combination thereof.

An exosome for analysis for prostate cancer can be detected with one or more binding agents including, but not limited to, PSA, PSMA, TMPRSS2, mAB 5D4, XPSM-A9, XPSM-A10, Galectin-3, E-selectin, Galectin-1, or E4 (IgG2a kappa), or any combination thereof.

An exosome for analysis for melanoma can be detected with one or more binding agents including, but not limited to, Tremelimumab (anti-CTLA4), Ipilimumumab (anti-CTLA4), CTLA-4 aptamers, STAT-3 peptide aptamers, Galectin-1, Galectin-3, or PNA, or any combination thereof.

An exosome for analysis for pancreatic cancer can be detected with one or more binding agents including, but not limited to, H38-15 (anti-HGF) aptamer, H38-21(anti-HGF) aptamer, Matuzumab, Cetuximanb, or Bevacizumab, or any combination thereof.

An exosome for analysis for brain cancer can be detected with one or more binding agents including, but not limited to, aptamer III.1 (pigpen) and/or TTA1 (Tenascin-C) aptamer, or any combination thereof.

An exosome for analysis for psoriasis can be detected with one or more binding agents including, but not limited to, E-selectin, ICAM-1, VLA-4, VCAM-1, alphaEbeta7, or any combination thereof.

An exosome for analysis for cardiovascular disease (CVD) can be detected with one or more binding agents including, but not limited to, RB007 (factor IXA aptamer), ARC1779 (anti VWF) aptamer, or LOX1, or any combination thereof.

An exosome for analysis for hematological malignancies can be detected with one or more binding agents including, but not limited to, anti-CD20 and/or anti-CD52, or any combination thereof.

An exosome for analysis for B-cell chronic lymphocytic leukemias can be detected with one or more binding agents including, but not limited to, Rituximab, Alemtuzumab, Apt48 (BCL6), R0-60, or D-R15-8, or any combination thereof.

An exosome for analysis for B-cell lymphoma can be detected with one or more binding agents including, but not limited to, Ibritumomab, Tositumomab, Anti-CD20 Antibodies, Alemtuzumab, Galiximab, Anti-CD40 Antibodies, Epratuzumab, Lumiliximab, HulD10, Galectin-3, or Apt48, or any combination thereof.

An exosome for analysis for Burkitt's lymphoma can be detected with one or more binding agents including, but not limited to, TD05 aptamer, IgM mAB (38-13), or any combination thereof.

An exosome for analysis for cervical cancer can be detected with one or more binding agents including, but not limited to, Galectin-9 and/or HPVE7 aptamer, or any combination thereof.

An exosome for analysis for endometrial cancer can be detected with one or more binding agents including, but not limited to, Galectin-1 or any combinations of binding agents specific for endometrial cancer.

An exosome for analysis for head and neck cancer can be detected with one or more binding agents including, but not limited to, (111)In-cMAb U36, anti-LOXL4, U36, BIWA-1, BIWA-2, BIWA-4, or BIWA-8, or any combination thereof.

An exosome for analysis for IBD can be detected with one or more binding agents including, but not limited to, ACCA (anti-glycan Ab), ALCA (anti-glycan Ab), or AMCA (anti-glycan Ab), or any combination thereof.

An exosome for analysis for diabetes can be detected with one or more binding agents including, but not limited to, RBP4 aptamer or any combination of binding agents specific for diabetes.

An exosome for analysis for fibromyalgia can be detected with one or more binding agents including, but not limited to, L-selectin or any combination of binding agents specific for fibromyalgia.

An exosome for analysis for multiple sclerosis (MS) can be detected with one or more binding agents including, but not limited to, Natalizumab (Tysabri) or any combination of binding agents specific for MS.

In addition, An exosome for analysis for rheumatic disease can be detected with one or more binding agents including, but not limited to, Rituximab (anti-CD20 Ab) and/or Keliximab (anti-CD4 Ab), or any combination of binding agents specific for rheumatic disease.

An exosome for analysis for Alzheimer disease can be detected with one or more binding agents including, but not limited to, TH14-BACE1 aptapers, S10-BACE1 aptapers, anti-Abeta, Bapineuzumab (AAB-001)-Elan, LY2062430 (anti-amyloid beta Ab)-Eli Lilly, or BACE1-Anti sense, or any combination thereof.

An exosome for analysis for Prion specific diseases can be detected with one or more binding agents including, but not limited to, rhuPrP(c) aptamer, DP7 aptamer, Thioaptamer 97, SAF-93 aptamer, 15B3 (anti-PrPSc Ab), monoclonal anti PrPSc antibody P1:1, 1.5D7, 1.6F4 Abs, mab 14D3, mab 4F2, mab 8G8, or mab 12F10, or any combination thereof.

An exosome for analysis for sepsis can be detected with one or more binding agents including, but not limited to, HA-1A mAb, E-5 mAb, TNF-alpha MAb, Afelimomab, or E-selectin, or any combination thereof.

An exosome for analysis for schizophrenia can be detected with one or more binding agents including, but not limited to, L-selectin and/or N-CAM, or any combination of binding agents specific for schizophrenia.

An exosome for analysis for depression can be detected with one or more binding agents including, but not limited to, GPIb or any combination of binding agents specific for depression.

An exosome for analysis for GIST can be detected with one or more binding agents including, but not limited to, ANTI-DOG1 Ab or any combination of binding agents specific for GIST.

An exosome for analysis for esophageal cancer can be detected with one or more binding agents including, but not limited to, CaSR binding agent or any combination of binding agents specific for esophageal cancer.

An exosome for analysis for gastric cancer can be detected with one or more binding agents including, but not limited to, Calpain nCL-2 binding agent and/or drebrin binding agent, or any combination of binding agents specific for gastric cancer.

An exosome for analysis for COPD can be detected with one or more binding agents including, but not limited to, CXCR3 binding agent, CCR5 binding agent, or CXCR6 binding agent, or any combination of binding agents specific for COPD.

An exosome for analysis for asthma can be detected with one or more binding agents including, but not limited to, VIP binding agent, PACAP binding agent, CGRP binding agent, NT3 binding agent, YKL-40 binding agent, S-nitrosothiols, SCCA2 binding agent, PAI binding agent, amphiregulin binding agent, or Periostin binding agent, or any combination of binding agents specific for asthma.

An exosome for analysis for vulnerable plaque can be detected with one or more binding agents including, but not limited to, Gd-DTPA-g-mimRGD (Alpha v Beta 3 integrin binding peptide), or MMP-9 binding agent, or any combination of binding agents specific for vulnerable plaque.

An exosome for analysis for ovarian cancer can be detected with one or more binding agents including, but not limited to, (90) Y-muHMFG1 binding agent and/or OC125 (anti-CA125 antibody), or any combination of binding agents specific for ovarian cancer.

The binding agent can be for a general exosome marker, or “housekeeping protein” or antigen, such as CD9, CD63, or CD81. For example, the binding agent can be an antibody for CD9, CD63, or CD81. The binding agent can also be for other exosomal proteins, such as for prostate specific exosomes, or cancer specific exosomes, such as PCSA, PSMA, EpCam, B7H3, or STEAP. For example, the binding agent can be an antibody for PCSA, PSMA, EpCam, B7H3, or STEAP.

Furthermore, additional cellular binding partners or binding agents may be identified by any conventional methods known in the art, or as described herein, and may additionally be used as a diagnostic, prognostic or therapy-related marker. Bio-Signatures: Prostate Cancer, Colon Cancer and Ovarian Cancer

Prostate Cancer

An exosome bio-signature can be used to characterize prostate cancer. As described above, a bio-signature for prostate cancer can comprise a binding agent associated with prostate cancer (for example, as shown in FIG. 2), and one or more additional biomarkers, such as shown in FIG. 19. For example, a bio-signature for prostate cancer can comprise a binding agent to PSA, PSMA, TMPRSS2, mAB 5D4, XPSM-A9, XPSM-A10, Galectin-3, E-selectin, Galectin-1, E4 (IgG2a kappa), or any combination thereof, with one or more additional biomarkers, such as one or more miRNA, one or more DNA, one or more additional peptide, protein, or antigen associated with prostate cancer, such as, but not limited to, those shown in FIG. 19.

A bio-signature for prostate cancer can comprise an antigen associated with prostate cancer (for example, as shown in FIG. 1), and one or more additional biomarkers, such as shown in FIG. 19. A bio-signature for prostate cancer can comprise one or more antigens associated with prostate cancer, such as, but not limited to, KIA1, intact fibronectin, PSA, TMPRSS2, FASLG, TNFSF10, PSMA, NGEP, IL-7RI, CSCR4, CysLT1R, TRPM8, Kv1.3, TRPV6, TRPM8, PSGR, MISIIR, or any combination thereof. The bio-signature for prostate cancer can comprise one or more of the aforementioned antigens and one or more additional biomarkers, such as, but not limited to miRNA, mRNA, DNA, or any combination thereof.

A bio-signature for prostate cancer can also comprise one or more antigens associated with prostate cancer, such as, but not limited to, KIA1, intact fibronectin, PSA, TMPRSS2, FASLG, TNFSF10, PSMA, NGEP, IL-7RI, CSCR4, CysLT1R, TRPM8, Kv1.3, TRPV6, TRPM8, PSGR, MISIIR, or any combination thereof, and one or more miRNA biomarkers, such as, but not limited to, miR-202, miR-210, miR-296, miR-320, miR-370, miR-373, miR-498, miR-503, miR-184, miR-198, miR-302c, miR-345, miR-491, miR-513, miR-32, miR-182, miR-31, miR-26a-1/2, miR-200c, miR-375, miR-196a-1/2, miR-370, miR-425, miR-425, miR-194-1/2, miR-181a-1/2, miR-34b, let-7i, miR-188, miR-25, miR-106b, miR-449, miR-99b, miR-93, miR-92-1/2, miR-125a, miR-141, let-7a, let-7b, let-7c, let-7d, let-7g, miR-16, miR-23a, miR-23b, miR-26a, miR-92, miR-99a, miR-103, miR-125a, miR-125b, miR-143, miR-145, miR-195, miR-199, miR-221, miR-222, miR-497, let-7f, miR-19b, miR-22, miR-26b, miR-27a, miR-27b, miR-29a, miR-29b, miR-30_{—}5p, miR-30c, miR-100, miR-141, miR-148a, miR-205, miR-520h, miR-494, miR-490, miR-133a-1, miR-1-2, miR-218-2, miR-220, miR-128a, miR-221, miR-499, miR-329, miR-340, miR-345, miR-410, miR-126, miR-205, miR-7-1/2, miR-145, miR-34a, miR-487, or let-7b, or any combination thereof.

Furthermore, the miRNA for a prostate cancer bio-signature can be a miRNA that interacts with PFKFB3, RHAMM (HMMR), cDNA FLJ42103, ASPM, CENPF, NCAPG, Androgen Receptor, EGFR, HSP90, SPARC, DNMT3B, GART, MGMT, SSTR3, TOP2B, or any combination thereof, such as those described herein and depicted in FIG. 60. The miRNA can also be miR-9, miR-629, miR-141, miR-671-3p, miR-491, miR-182, miR-125a-3p, miR-324-5p, miR-148B, miR-222, or any combination thereof.

The bio-signature for prostate cancer can comprise one or more antigens associated with prostate cancer, such as, but not limited to, KIA1, intact fibronectin, PSA, TMPRSS2, FASLG, TNFSF10, PSMA, NGEP, IL-7RI, CSCR4, CysLT1R, TRPM8, Kv1.3, TRPV6, TRPM8, PSGR, MISIIR, or any combination thereof, and one or more additional biomarkers such as, but not limited to, the aforementioned miRNAs, mRNAs (such as, but not limited to, AR or PCA3), snoRNA (such as, but not limited to, U50) or any combination thereof.

The bio-signature can also comprise one or more gene fusions, such as ACSL3-ETV1, C150RF21-ETV1, FLJ35294-ETV1, HERV-ETV1, TMPRSS2-ERG, TMPRSS2-ETV1/4/5, TMPRSS2-ETV4/5, SLC5A3-ERG, SLC5A3-ETV1, SLC5A3-ETV5 or KLK2-ETV4.

An exosome can be isolated and assayed for one or more miRNA and one or more antigens associated with prostate cancer to provide a diagnostic, prognostic or theranostic profile, such as the stage of the cancer, the efficacy of the cancer, or other characteristics of the cancer. Alternatively, the exosome can be directly assayed from a sample, such that the exosomes are not purified or concentrated prior to assaying for one or more miRNA or antigens associated with prostate cancer.

As depicted in FIGS. 68A-R, a prostate cancer bio-signature can comprise assaying EpCam, CD63, CD81, CD9, or any combination thereof, of an exosome. The prostate cancer bio-signature can comprise detection of EpCam, CD9, CD63, CD81, PCSA or any combination thereof. For example, the prostate cancer bio-signature can comprise EpCam, CD9, CD63 and CD81 or PCSA, CD9, CD63 and CD81 (see for example, FIGS. 70A-D). The prostate cancer bio-signature can also comprise PCSA, PSMA, B7H3, or any combination thereof (see for example, FIGS. 70E-F).

Furthermore, assessing a plurality of biomarkers can provide increased sensitivity, specificity, or signal intensity, as compared to assessing less than a plurality of biomarkers. For example, assessing PSMA and B7H3 can provide increased sensitivity in detection as compared to assessing PSMA or B7H3 alone.

Assessing CD9 and CD63 can provide increased sensitivity in detection as compared to assessing CD or CD63 alone.

Prostate cancer can also be characterized based on meeting at least 1, 2, 3, 4, 5, 6, 7, 8, 9, or 10 criteria. For example, a number of different criteria can be used: 1) if the amount of exosomes in a sample from a subject is higher than a reference value; 2) if the amount of prostate cell derived exosomes is higher than a reference value; and 3) if the amount of exosomes with one or more cancer specific biomarkers is higher than a reference value, the subject is diagnosed with prostate cancer. The method can further include a quality control measure, such that the subject is diagnosed with prostate cancer if the determination of the other criteria

The prostate cancer can be characterizing using one or more processes disclosed herein with at least 60, 61, 62, 63, 64, 65, 66, 67, 68, 69, or 70% sensitivity. The prostate cancer can be characterized with at least 80, 81, 82, 83, 84, 85, 86, or 87% sensitivity. For example, the prostate cancer can be characterized with at least 87.1, 87.2, 87.3, 87.4, 87.5, 87.6, 87.7, 87.8, 87.9, 88.0, or 89% sensitivity, such as with at least 90% sensitivity, such as at least 91, 92, 93, 94, 95, 96, 97, 98, 99 or 100% sensitivity.

The prostate cancer of a subject can also be characterized with at least 70, 71, 72, 73, 74, 75, 76, 77, 78, 79, 80, 81, 82, 83, 84, 85, 86, 87, 88, 89, 90, 91, 92, 93, 94, 95, 96, or 97% specificity, such as with at least 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9 or 100% specificity.

The prostate cancer can also be characterized with at least 70% sensitivity and at least 80, 90, 95, 99, or 100% specificity; at least 80% sensitivity and at least 80, 85, 90, 95, 99, or 100% specificity; at least 85% sensitivity and at least 80, 85, 90, 95, 99, or 100% specificity; at least 86% sensitivity and at least 80, 85, 90, 95, 99, or 100% specificity; at least 87% sensitivity and at least 80, 85, 90, 95, 99, or 100% specificity; at least 88% sensitivity and at least 80, 85, 90, 95, 99, or 100% specificity; at least 89% sensitivity and at least 80, 85, 90, 95, 99, or 100% specificity; at least 90% sensitivity and at least 80, 85, 90, 95, 99, or 100% specificity; at least 95% sensitivity and at least 80, 85, 90, 95, 99, or 100% specificity; at least 99% sensitivity and at least 80, 85, 90, 95, 99, or 100% specificity; or at least 100% sensitivity and at least 80, 85, 90, 95, 99, or 100% specificity.

Furthermore, the confidence level for determining the specificity, sensitivity, or both, may be with at least 90, 91, 92, 93, 94, 95, 96, 97, 98, or 99% confidence.

Colon Cancer

A colon cancer bio-signature can comprise any one or more antigens for colon cancer as listed in FIG. 1, any one or more binding agents associated with isolating an exosome for characterizing colon cancer (for example, as shown in FIG. 2), any one or more additional biomarkers, such as shown in FIG. 6.

The bio-signature can comprise one or more miRNA selected from the group consisting of miR-24-1, miR-29b-2, miR-20a, miR-10a, miR-32, miR-203, miR-106a, miR-17-5p, miR-30c, miR-223, miR-126, miR-128b, miR-21, miR-24-2, miR-99b, miR-155, miR-213, miR-150, miR-107, miR-191, miR-221, miR-20a, miR-510, miR-92, miR-513, miR-19a, miR-21, miR-20, miR-183, miR-96, miR-135b, miR-31, miR-21, miR-92, miR-222, miR-181b, miR-210, miR-20a, miR-106a, miR-93, miR-335, miR-338, miR-133b, miR-346, miR-106b, miR-153a, miR-219, miR-34a, miR-99b, miR-185, miR-223, miR-211, miR-135a, miR-127, miR-203, miR-212, miR-95, or miR-17-5p, or any combination thereof. The bio-signature can also comprise one or more underexpressed miRs such as miR-143, miR-145, miR-143, miR-126, miR-34b, miR-34c, let-7, miR-9-3, miR-34a, miR-145, miR-455, miR-484, miR-101, miR-145, miR-133b, miR-129, miR-124a, miR-30-3p, miR-328, miR-106a, miR-17-5p, miR-342, miR-192, miR-1, miR-34b, miR-215, miR-192, miR-301, miR-324-5p, miR-30a-3p, miR-34c, miR-331, and miR-148b.

The bio-signature can comprise assessing one or more genes, such as EFNB1, ERCC1, HER2, VEGF, and EGFR. A biomarker mutation for colon cancer that can be assessed in an exosome can also include one or more mutations of EGFR, KRAS, VEGFA, B-Raf, APC, or p53. The bio-signature can also comprise one or more proteins, ligands, or peptides that can be assessed of an exosome such as AFRs, Rabs, ADAM10, CD44, NG2, ephrin-B1, MIF, b-catenin, Junction, plakoglobin, glalectin-4, RACK1, tetrspanin-8, FasL, TRAIL, A33, CEA, EGFR, dipeptidase 1, hsc-70, tetraspanins, ESCRT, TS, PTEN, or TOPO1

An exosome can be isolated and assayed for to provide a diagnostic, prognostic or theranostic profile, such as the stage of the cancer, the efficacy of the cancer, or other characteristics of the cancer. Alternatively, the exosome can be directly assayed from a sample, such that the exosomes are not purified or concentrated prior to assaying for a bio-signature associated with colon cancer.

As depicted in FIGS. 69A-J, a colon cancer signature can comprise detection of EpCam, CD63, CD81, CD9, CD66, or any combination thereof, of an exosome. Furthermore, a colon cancer-bio-signature for various stages of cancer can comprise CD63, CD9, EpCam, or any combination thereof (see for example, FIGS. 71A-F and 72A-F). For example, the bio-signature can comprise CD9 and EpCam.

The colon cancer can be characterizing using one or more processes disclosed herein with at least 60, 61, 62, 63, 64, 65, 66, 67, 68, 69, or 70% sensitivity. The colon cancer can be characterized with at least 80, 81, 82, 83, 84, 85, 86, or 87% sensitivity. For example, the colon cancer can be characterized with at least 87.1, 87.2, 87.3, 87.4, 87.5, 87.6, 87.7, 87.8, 87.9, 88.0, or 89% sensitivity, such as with at least 90% sensitivity, such as at least 91, 92, 93, 94, 95, 96, 97, 98, 99 or 100% sensitivity.

The colon cancer of a subject can also be characterized with at least 70, 71, 72, 73, 74, 75, 76, 77, 78, 79, 80, 81, 82, 83, 84, 85, 86, 87, 88, 89, 90, 91, 92, 93, 94, 95, 96, or 97% specificity, such as with at least 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9 or 100% specificity.

The colon cancer can also be characterized with at least 70% sensitivity and at least 80, 90, 95, 99, or 100% specificity; at least 80% sensitivity and at least 80, 85, 90, 95, 99, or 100% specificity; at least 85% sensitivity and at least 80, 85, 90, 95, 99, or 100% specificity; at least 86% sensitivity and at least 80, 85, 90, 95, 99, or 100% specificity; at least 87% sensitivity and at least 80, 85, 90, 95, 99, or 100% specificity; at least 88% sensitivity and at least 80, 85, 90, 95, 99, or 100% specificity; at least 89% sensitivity and at least 80, 85, 90, 95, 99, or 100% specificity; at least 90% sensitivity and at least 80, 85, 90, 95, 99, or 100% specificity; at least 95% sensitivity and at least 80, 85, 90, 95, 99, or 100% specificity; at least 99% sensitivity and at least 80, 85, 90, 95, 99, or 100% specificity; or at least 100% sensitivity and at least 80, 85, 90, 95, 99, or 100% specificity.

Furthermore, the confidence level for determining the specificity, sensitivity, or both, may be with at least 90, 91, 92, 93, 94, 95, 96, 97, 98, or 99% confidence.

Ovarian Cancer

A bio-signature for characterizing ovarian cancer can comprise an antigen associated with ovarian cancer (for example, as shown in FIG. 1), and one or more additional biomarkers, such as shown in FIG. 4. In one embodiment, a bio-signature for ovarian cancer can comprise one or more antigens associated with ovarian cancer, such as, but not limited to, CD24, CA125, VEGF1, VEGFR2, HER2, MISIIR, or any combination thereof. The bio-signature for ovarian cancer can comprise one or more of the aforementioned antigens and one or more additional biomarker, such as, but not limited to miRNA, mRNA, DNA, or any combination thereof. The bio-signature for ovarian cancer can comprise one or more antigens associated with ovarian cancer, such as, but not limited to, CD24, CA125, VEGF1, VEGFR2, HER2, MISIIR, or any combination thereof, with one or more miRNA biomarkers, such as, but not limited to, miR-200a, miR-141, miR-200c, miR-200b, miR-21, miR-141, miR-200a, miR-200b, miR-200c, miR-203, miR-205, miR-214, miR-215, miR-199a, miR-140, miR-145, miR-125b-1, or any combination thereof.

A bio-signature for ovarian cancer can comprise one or more antigens associated with ovarian cancer, such as, but not limited to, CD24, CA125, VEGF1, VEGFR2, HER2, MISIIR, or any combination thereof, with one or more miRNA biomarkers (such as the aforementioned miRNA), mRNAs (such as, but not limited to, ERCC1, ER, TOPO1, TOP2A, AR, PTEN, HER2/neu, EGFR), mutations (including, but not limited to, those relating to KRAS and/or B-Raf) or any combination thereof.

An exosome can be isolated and assayed for one or more miRNA and one or more antigens associated with ovarian cancer to provide a diagnostic, prognostic or theranostic profile. Alternatively, the exosome can be directly assayed from a sample, such that the exosomes are not purified or concentrated prior to assaying for one or more miRNA or antigens associated with ovarian cancer.

### Bio-Signatures: Assessing Organ Transplant Rejection and Autoimmune Conditions

An exosome can also be used for determining phenotypes such as organ distress and/or organ transplant rejection. As used herein organ transplant includes partial organ or tissue transplant. The presence, absence or levels of one or more biomarkers present in exosomes is assessed to monitor organ rejection or success. The level, or amount, of exosomes in the sample can also be used to assess organ rejection or success. The assessment can be determined with at least 80, 81, 82, 83, 84, 85, 86, 87, 88, 89, 90, 91, 92, 93, 94, 95, 96, 97, 98, or 99% specificity, sensitivity, or both. For example, the assessment can be determined with at least 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 998.2, 99.3, 99.4, 99.5, 99.6, 99.7, 99.8, 99.9% sensitivity, specificity, or both

The exosome can be purified or concentrated prior to analysis. Alternatively, the level, or amount, of exosomes can be directly assayed from a sample, without prior purification or concentration. The exosome quantitated can be a cell-of-origin specific exosome. For example, a cell or tissue-specific exosome can be isolated using one or more binding agents specific for a particular organ. The cell-of-origin specific exosome can be assessed for one or more molecular features, such as one or more biomarkers associated with organ distress or organ transplant rejection. The presence, absence or levels of one or more biomarkers present in an isolated cell-of-origin specific exosome can be assessed to monitor organ rejection or success.

One or more exosomes can be analyzed for the assessment, detection or diagnosis of the rejection of a tissue or organ transplant by a subject. The tissue or organ transplant rejection can be hyperacute, acute, or chronic rejection. The exosome can also be analyzed for the assessment, detection or diagnosis of graft versus host disease in a subject. The subject can be the recipient of an autogenic, allogenic or xenogenic tissue or organ transplant.

The exosome can also be analyzed to detect the rejection of a tissue or organ transplant. The exosome may be produced by the tissue or organ transplant. Such tissues or organs include, but are not limited to, a heart, lung, pancreas, kidney, eye, cornea, muscle, bone marrow, skin, cartilage, bone, appendages, hair, face, tendon, stomach, intestine, vein, artery, differentiated cells, partially differentiated cells or stem cells.

The exosome can comprise at least one biomarker which is used to assess, diagnose or determine the probability or occurrence of rejection of a tissue or organ transplant by a subject. A biomarker can also be used to assess, diagnose or detect graft versus host disease in a subject. The biomarker can be a protein, a polysaccharide, a fatty acid or a nucleic acid (such as DNA or RNA). The biomarker can be associated with the rejection of a specific tissue or organ or systemic organ failure. More than one biomarker can be analyzed, for example, one or more proteins marker can be analyzed in combination with one or more nucleic acid markers. The biomarker may be an intracellular or extracellular marker.

The exosome can also be analyzed for at least one marker for the assessment, detection or diagnosis of cell apoptosis or necrosis associated with, or the causation of, rejection of a tissue or organ transplant by a subject.

The presence of a biomarker can be indicative of the rejection of a tissue or an organ by a subject, wherein the biomarker includes, but is not limited to, CD40, CD40 ligand, N-acetylmuramoyl-L-alanine amidase precursor, adiponectin, AMBP protein precursor, C4b-binding protein a-chain precursor, ceruloplasmin precursor, complement C3 precursor, complement component C9 precursor, complement factor D precursor, alpha1-B-glycoprotein, beta2-glycoprotein I precursor, heparin cofactor II precursor, Immunoglobulin mu chain C region protein, Leucine-rich alpha2-glycoprotein precursor, pigment epithelium-derived factor precursor, plasma retinol-binding protein precursor, translation initiation factor 3 subunit 10, ribosomal protein L7, beta-transducin, 1-TRAF, or lysyl-tRNA synthetase.

Rejection of a kidney by a subject can also be detected by analyzing exosomes for the presence of beta-transducin. Rejection of transplanted tissue can also be detected by isolating a cell-of-origin specific exosome from CD40-expressing cells and detecting for the increase of Bcl-2 or TNFalpha.

Rejection of a liver transplant by a subject can be detected by analyzing the exosomesfor the presence of an F1 antigen marker. The F1 antigen is, without being bound to theory, specific to liver to and can be used to detect an increase in liver cell-of-origin specific exosomes. This increase can be used as an early indication of organ distress/rejection.

Bronchiolitis obliterans due to bone marrow and/or lung transplantation or other causes, or graft atherosclerosis/graft phlebosclerosis can also be diagnosed by the analysis of an exosome.

An exosome can also be analyzed for the detection, diagnosis or assessment of an autoimmune or other immunological reaction-related phenotypes in a subject. Examples of such a disorder include, but are not limited to, systemic lupus erythematosus (SLE), discoid lupus, lupus nephritis, sarcoidosis, inflammatory arthritis, including juvenile arthritis, rheumatoid arthritis, psoriatic arthritis, Reiter's syndrome, ankylosing spondylitis, and gouty arthritis, multiple sclerosis, hyper IgE syndrome, polyarteritis nodosa, primary biliary cirrhosis, inflammatory bowel disease, Crohn's disease, celiac's disease (gluten-sensitive enteropathy), autoimmune hepatitis, pernicious anemia, autoimmune hemolytic anemia, psoriasis, scleroderma, myasthenia gravis, autoimmune thrombocytopenic purpura, autoimmune thyroiditis, Grave's disease, Hasimoto's thyroiditis, immune complex disease, chronic fatigue immune dysfunction syndrome (CFIDS), polymyositis and dermatomyositis, cryoglobulinemia, thrombolysis, cardiomyopathy, pemphigus vulgaris, pulmonary interstitial fibrosis, asthma, Churg-Strauss syndrome (allergic granulomatosis), atopic dermatitis, allergic and irritant contact dermatitis, urtecaria, IgE-mediated allergy, atherosclerosis, vasculitis, idiopathic inflammatory myopathies, hemolytic disease, Alzheimer's disease, chronic inflammatory demyelinating polyneuropathy and AIDs.

One or more biomarkers from the exosome can be used to assess, diagnose or determine the probability of the occurrence of an autoimmune or other immunological reaction-related disorder in a subject. The biomarker can be a protein, a polysaccharide, a fatty acid or a nucleic acid (such as DNA or RNA). The biomarker can be associated with a specific autoimmune disorder, a systemic autoimmune disorder, or other immunological reaction-related disorder. More than one biomarker can be analyzed. For example one or more protein markers can be analyzed in combination with one or more nucleic acid markers. The biomarker can be an intracellular or extracellular marker. The biomarker can also be used to detect, diagnose or assess inflammation.

Analysis of an exosome from subjects can be used identify subjects with inflammation associated with asthma, sarcoidosis, emphysema, cystic fibrosis, idiopathic pulmonary fibrosis, chronic bronchitis, allergic rhinitis and allergic diseases of the lung such as hypersensitivity pneumonitis, eosinophilic pneumonia, as well as pulmonary fibrosis resulting from collagen, vascular, and autoimmune diseases such as rheumatoid arthritis.

### Exosome Compositions

Also provided herein is an isolated exosome with a particular bio-signature. The isolated exosome can comprise one or more biomarkers or bio-signatures specific for specific cell type, or for characterizing a phenotype, such as described above. For example, the isolated exosome can comprise one or more biomarkers, such as CD63, EpCam, CD81, CD9, PCSA, PSMA, B7H3, TNFR, MFG-E8, Rab, STEAP, 5T4, or CD59. The isolated exosome can have the one or more biomarkers on its surface of within the exosome. The isolated exosome can also comprise one or more miRNAs, such as miR-9, miR-629, miR-141, miR-671-3p, miR-491, miR-182, miR-125a-3p, miR-324-5p, miR-148B, or miR-222. An isolated exosome can comprise a biomarker such as CD66, and further comprise one or more biomarkers selected from the group consisting of: EpCam, CD63, or CD9. An isolated exosome can also comprise a fusion gene or protein, such as TMRSSG2:ERG.

An isolated exosome can also comprise one or more biomarkers, wherein the expression level of the one or more biomarkers is higher, lower, or the same for an isolated exosome as compared to an isolated exosome derived from a normal cell (ie. a cell derived from a subject without a phenotype of interest). For example, an isolated exosome can comprise one or more biomarkers selected from the group consisting of: B7H3, PSCA, MFG-E8, Rab, STEAP, PSMA, PCSA, 5T4, miR-9, miR-629, miR-141, miR-671-3p, miR-491, miR-182, miR-125a-3p, miR-324-5p, miR-148b, and miR-222, wherein the expression level of the one or more biomarkers is higher for an isolated exosome as compared to an isolated exosome derived from a normal cell. The isolated exosome can comprise at least 2, 3, 4, 5, 6, 7, 8, 9, 10, 11, 13, 14, 15, 16, 17, 18, or 19 of the biomarkers selected from the group. The isolated exosome can further comprising one or more biomarkers selected from the group consisting of: EpCam, CD63, CD59, CD81, or CD9.

An isolated exosome can comprise the biomarkers PCSA, EpCam, CD63, and CD8; the biomarkers PCSA, EpCam, B7H3 and PSMA. An isolated exosome can comprise the biomarkers miR-9, miR-629, miR-141, miR-671-3p, miR-491, miR-182, miR-125a-3p, miR-324-5p, miR-148b, and miR-222.

A composition comprising an isolated exosome is also provided herein. The composition can comprise one or more isolated exosomes. For example, the composition can comprise a plurality of exosomes, or one or more populations of exosomes.

The composition can be substantially enriched for exosomes. For example, the composition can be substantially absent of cellular debris, cells, or non-exosomal proteins, peptides, or nucleic acids (such as biological molecules not contained within the exosomes). The cellular debris, cells, or non-exosomal proteins, peptides, or nucleic acids, can be present in a biological sample along with exosomes. A composition can be substantially absent of cellular debris, cells, or non-exosomal proteins, peptides, or nucleic acids (such as biological molecules not contained within the exosomes), can be obtained by any method disclosed herein, such as through the use of one or more binding agents or capture agents for one or more exosomes. The exosomes can comprise at least 30, 40, 50, 60, 70, 80, 90, 95 or 99% of the total composition, by weight or by mass. The exosomes of the composition can be a heterogeneous or homogeneous population of exosomes. For example, a homogeneous population of exosomes comprises exosomes that are homogeneous as to one or more properties or characteristics. For example, the one or more characteristics can be selected from a group consisting of: one or more of the same biomarkers, a substantially similar or identical bio-signature, derived from the same cell type, exosomes of a particular size, and a combination thereof.

Thus, in some embodiments, the composition comprises a substantially enriched population of exosomes. The composition can be enriched for a population of exosomes that are at least 30, 40, 50, 60, 70, 80, 90, 95 or 99% homogeneous as to one or more properties or characteristics. For example, the one or more characteristics can be selected from a group consisting of: one or more of the same biomarkers, a substantially similar or identical bio-signature, derived from the same cell type, exosomes of a particular size, and a combination thereof. For example, the population of exosomes can be homogeneous by all having a particular bio-signature, having the same biomarker, having the same biomarker combination, or derived from the same cell type. In some embodients, the composition comprises a substantially homogeneous population of exosomes, such as a population with a specific bio-signature, derived from a specific cell, or both.

The population of exosome can comprise one or more of the same biomarkers. The biomarker can be any component present in an exosome or on the exosome, such as any nucleic acid (e.g. RNA or DNA), protein, peptide, polypeptide, antigen, lipid, carbohydrate, or proteoglycan. For example, each exosome in a population can comprise the same or identical one or more biomarkers. In some embodiments, each exosome in the population comprises the same 1, 2, 3, 4, 5, 6, 3, 4, 5, 6, 7, 8, 9, 10, 11, 12, 13, 14, 15, 16, 17, 18, 19, 20, 25, 50, 75 or 100 biomarkers. The one or more biomarkers can be selected from FIGS. 1, 3-60.

The exosome population comprising the same or identical biomarker can refer to each exosome in the population having the same presence or absence, expression level, mutational state, or modification of the biomarker. For example, an enriched population of exosome can comprise exosomes, wherein each exosome has the same biomarker present, the same biomarker absent, the same expression level of a biomarker, the same modification of a biomarker, or the same mutation of a biomarker. The same expression level of a biomarker can refer to a quantitative or qualitative measurement, such as the exosomes in the population underexpress, overexpress, or have the same expression level of a biomarker as compared to a reference level. Alternatively, the same expression level of a biomarker can be a numerical value representing the expression of a biomarker that is similar for each exosome in a population. For example the copy number of a miRNA, the amount of protein, or the level of mRNA of each exosome can be quantitatively similar for each exosome in a population, such that the numerical amount of each exosome is ±1, 2, 3, 4, 5, 6, 7, 8, 9, 10, 15, or 20% from the amount in each other exosome in the population, as such variations are appropriate.

In some embodiments, the composition comprises a substantially enriched population of exosomes, wherein the exosomes in the enriched population has a substantially similar or identical bio-signature. The bio-signature can comprise one or more exosomal characteristic such as the level or amount of exosomes, temporal evaluation of the variation in exosomal half-life, circulating exosomal half-life or exosomal metabolic half-life, or the activity of an exosome. The bio-signature can also comprise the presence or absence, expression level, mutational state, or modification of a biomarker, such as those described herein.

The bio-signature of each exosome in the population can be at least 30, 40, 50, 60, 70, 80, 90, 95, or 99% identical. In some embodiments, the bio-signature of each exosome is 100% identical. The bio-signature of each exosome in the enriched population can have the same 1, 2, 3, 4, 5, 6, 3, 4, 5, 6, 7, 8, 9, 10, 11, 12, 13, 14, 15, 16, 17, 18, 19, 20, 25, 50, 75 or 100 exosomal characteristics. For example, a bio-signature of an exosome in an enriched population can be the presence of a first biomarker, the presence of a second biomarker, and the underexpression of a third biomarker. Another exosome in the same population can be 100% identical, having the same first and second biomarkers present and underexpression of the third biomarker. Alternatively, an exosome in the same population can have the same first and second biomarkers, but not have underexpression of the third biomarker.

In some embodiments, the composition comprises a substantially enriched population of exosomes, wherein the exosomes are derived from the same cell type. For example, the exosomes can all be derived from cells of a specific tissue, cells from a specific tumor of interest or a diseased tissue of interest, circulating tumor cells, or cells of maternal or fetal origin. The exosomes can all be derived from tumor cells. The exosomes can all be derived from lung, pancreas, stomach, intestine, bladder, kidney, ovary, testis, skin, colorectal, breast, prostate, brain, esophagus, liver, placenta, or fetal cells.

The composition comprising a substantially enriched population of exosomes can also comprise exosomes are of a particular size. For example, the exosomes can all a diameter of greater than about 10, 20, or 30 nm. They can all have a diameter of about 30-1000 nm, about 30-800 nm, about 30-200 nm, or about 30-100 nm. In some embodiments, the exosomes can all have a diameter of less than about 10,000 nm, 1000 nm, 800 nm, 500 nm, 200 nm, 100 nm or 50 nm.

The population of exosomes homogeneous for one or more characteristics can comprises at least about 30, 40, 50, 60, 70, 80, 90, 95, or 99% of the total exosome population of the composition. In some embodiments, a composition comprising a substantially enriched population of exosomes comprises at least 2, 3, 4, 5, 10, 20, 25, 50, 100, 250, 500, or 1000 times the concentration of an exosome as compared to a concentration of the exosome in a biological sample from which the composition was derived. In yet other embodiments, the composition can further comprise a second enriched population of exosomes, wherein the population of exosomes is at least 30% homogeneous as to one or more characteristics, as described herein.

Multiplex analysis can be used to obtain a composition substantially enriched for more than one population of exosomes, such as at least 2, 3, 4, 5, 6, 7, 8, 9, 10 exosome populations. Each substantially enriched exosome population can comprise at least 5, 10, 15, 20, 25, 30, 35, 40, 45, 46, 47, 48, or 49% of the composition, by weight or by mass. In some embodiments, the substantially enriched exosome populations comprises at least about 30, 40, 50, 60, 70, 80, 90, 95, or 99% of the composition, by weight or by mass.

A substantially enriched population of exosomes can be obtained by using one or more methods, processes, or systems as disclosed herein. For example, isolation of a population of exosomes from a sample can be performed by using one or more binding agents for one or more biomarkers of an exosome, such as using two or more binding agents that target two or more biomarkers of an exosome. One or more capture agents can be used to obtain a substantially enriched population of exosomes. One or more detection agents can be used to identify a substantially enriched population of exosomes.

In one embodiment, a population of exosomes with a particular bio-signature is obtained by using one or more binding agents for the biomarkers of the bio-signature. The exosomes can be isolated resulting in a composition comprising a substantially enriched population of exosomes with the particular bio-signature. In another embodiment, a population of exosomes with a particular bio-signature of interest can be obtained by using one or more binding agents for biomarkers that are not a component of the bio-signature of interest. Thus, the binding agents can be used to remove the exosomes that do not have the bio-signature of interest and the resulting composition is substantially enriched for the population of exosomes with the particular bio-signature of interest. The resulting composition can be substantially absent of the exosomes comprising a biomarker for the binding agent.

### Detection System and Kits

Also provided is a detection system configured to determine one or more bio-signatures for an exosome. The detection system can be used to detect a heterogeneous population of exosomes or one or more homogeneous population of exosomes. The detection system can be configured to detect a plurality of exosomes, wherein at least a subset of said plurality of exosomes comprises a different bio-signature from another subset of said plurality of exosomes. The detection system detect at least 2, 3, 4, 5, 6, 7, 8, 9, 10, 15, 20, 25, 30, 40, 50, 60, 70, 80, 90, or 100 different subsets of exosomes, wherein each subset of exosomes comprises a different bio-signature. For example, a detection system, such as using one or more methods, processes, and compositions disclosed herein, can be used to detect at least 2, 3, 4, 5, 6, 7, 8, 9, 10, 15, 20, 25, 30, 40, 50, 60, 70, 80, 90, or 100 different populations of exosomes.

The detection system can be configured to assess at least 2, 3, 4, 5, 6, 7, 8, 9, 10, 15, 20, 25, 30, 40, 50, 60, 70, 80, 90, 100, 1000, 2500, 5000, 7500, 10,000, 100,000, 150,000, 200,000, 250,000, 300,000, 350,000, 400,000, 450,000, 500,000, 750,000, or 1,000,000 different biomarkers for one or more exosomes. In some embodiments, the one or more biomarkers are selected from FIG. 1, 3-60, or as disclosed herein. The detection system can be configured to assess a specific population of exosomes, such as exosomes from a specific cell-of-origin, or to assess a plurality of specific populations of exosomes, wherein each population of exosomes has a specific bio-signature.

The detection system can be a low density detection system or a high density detection system. For example, a low density detection system can detect up to 1, 2, 3, 4, 5, 6, 7, 8, 9, or 10 different exosome populations, whereas a high density detection system can detect at least about 15, 20, 25, 50, or 100 different exosome populations In another embodiment, a low density detection system can detect up to about 100, 200, 300, 400, or 500 different biomarkers, whereas a high density detection system can detect at least about 750, 1000, 2000, 3000, 4000, 5000, 6000, 7000, 8000, 9,000, 10,000, 15,000, 20,000, 25,000, 50,000, or 100,000 different biomarkers. In yet another embodiment, a low density detection system can detect up to about 100, 200, 300, 400, or 500 different bio-signatures or biomarker combinations, whereas a high density detection system can detect at least about 750, 1000, 2000, 3000, 4000, 5000, 6000, 7000, 8000, 9,000, 10,000, 15,000, 20,000, 25,000, 50,000, or 100,000 bio-signatures or biomarker combinations.

The detection system can comprise a probe that selectively hybridizes to an exosome. The detection system can comprise a plurality of probes to detect an exosome. In some embodiments, a plurality of probes is used to detect the amount of exosomes in a heterogeneous population of exosomes. In yet other embodiments, a plurality of probes is used to detect a homogeneous population of exosomes. A plurality of probes can be used to isolate or detect at least two different subsets of exosomes, wherein each subset of exosomes comprises a different bio-signature.

A detection system, such as using one or more methods, processes, and compositions disclosed herein, can comprise a plurality of probes configured to detect, or isolate, such as using one or more methods, processes, and compositions disclosed herein at least 2, 3, 4, 5, 6, 7, 8, 9, 10, 15, 20, 25, 30, 40, 50, 60, 70, 80, 90, or 100 different subsets of exosomes, wherein each subset of exosomes comprises a different bio-signature.

For example, a detection system can comprise a plurality of probes configured to detect at least 2, 3, 4, 5, 6, 7, 8, 9, 10, 15, 20, 25, 30, 40, 50, 60, 70, 80, 90, or 100 different populations of exosomes. The detection system can comprise a plurality of probes configured to selectively hybridize to at least 2, 3, 4, 5, 6, 7, 8, 9, 10, 15, 20, 25, 30, 40, 50, 60, 70, 80, 90, 100, 1000, 2500, 5000, 7500, 10,000, 100,000, 150,000, 200,000, 250,000, 300,000, 350,000, 400,000, 450,000, 500,000, 750,000, or 1,000,000 different biomarkers for one or more exosomes. In some embodiments, the one or more biomarkers are selected from FIG. 1, 3-60, or as disclosed herein. The plurality of probes can be configured to assess a specific population of exosomes, such as exosomes from a specific cell-of-origin, or to assess a plurality of specific populations of exosomes, wherein each population of exosomes has a specific bio-signature.

The detection system can be a low density detection system or a high density detection system comprising probes to detect exosomes. For example, a low density detection system can comprise probes to detect up to 1, 2, 3, 4, 5, 6, 7, 8, 9, or 10 different exosome populations, whereas a high density detection system can comprise probes to detect at least about 15, 20, 25, 50, or 100 different exosome populations In another embodiment, a low density detection system can comprise probes to detect up to about 100, 200, 300, 400, or 500 different biomarkers, whereas a high density detection system can comprise probes to detect at least about 750, 1000, 2000, 3000, 4000, 5000, 6000, 7000, 8000, 9,000, 10,000, 15,000, 20,000, 25,000, 50,000, or 100,000 different biomarkers. In yet another embodiment, a low density detection system can comprise probes to detect up to about 100, 200, 300, 400, or 500 different bio-signatures or biomarker combinations, whereas a high density detection system can comprise probes to detect at least about 750, 1000, 2000, 3000, 4000, 5000, 6000, 7000, 8000, 9,000, 10,000, 15,000, 20,000, 25,000, 50,000, or 100,000 bio-signatures or biomarker combinations.

The probes can be specific for detecting a specific exosome population, for example an exosome with a particular bio-signature, and as described above. A plurality of probes for detecting prostate specific exosomes is also provided. A plurality of probes can comprise probes for detecting one or more of the following biomarkers: CD9, PSCA, TNFR, CD63, MFG-E8, EpCAM, Rab, CD81, STEAP, PCSA, 5T4, EpCAM, PSMA, CD59, CD66, CD24 and B7H3. A plurality of probes for detecting Bcl-XL, ERCC1, Keratin 15, CD81/TAPA-1, CD9, Epithelial Specific Antigen (ESA), and Mast Cell Chymase can also be provided. A plurality of probes for detecting one or more miRNAs of an exosome can comprise probes for detecting one or more of the following miRNAs: miR-9, miR-629, miR-141, miR-671-3p, miR-491, miR-182, miR-125a-3p, miR-324-5p, miR-148b, and miR-222,

The probes may be attached to a solid substrate, such as an array or bead. Alternatively, the probes are not attached. The detection system may be an array based system, a sequencing system, a PCR-based system, or a bead-based system, such as described above. For example, the detection system can be a microfluidic device as described above.

The detection system may be part of a kit. Alternatively, the kit may comprise the one or more probe sets, or plurality of probes, as described herein. The kit may comprise probes for detecting an isolated exosome, a plurality of exosomes, such as exosomes in a heterogeneous population. The kit may comprise probes for detecting a homogeneous population of exosomes. For example, the kit may comprise probes for detecting a population of specific cell-of-origin exosomes, or exosomes with the same specific bio-signature.

### Portfolios

Portfolios of multiplexed markers to guide clinical decisions and disease detection and management can be established such that the combination of bio-signatures in the portfolio exhibit improved sensitivity and specificity relative to individual bio-signatures or randomly selected combinations of bio-signatures. In the context of the instant invention, the sensitivity of the portfolio can be reflected in the fold differences exhibited by a bio-signature's expression in the diseased state relative to the normal state. Specificity can be reflected in statistical measurements of the correlation of the signaling of gene expression, for example, with the condition of interest (e.g. standard deviation can be a used as such a measurement). In considering a group of bio-signature for inclusion in a portfolio, a small standard deviation in measurements correlates with greater specificity. Other measurements of variation such as correlation coefficients can also be used in this capacity.

When combining biomakers or bio-signatures in this invention In Vitro Diagnostic Multivariate Index Assays (IVDMIAs) guidelines and regulations may apply. IVDMIAs can apply to bio-signatures as defined as a set of 2 or more markers composed of any combination of genes, gene alterations, mutations, amplifications, deletions, polymorphisms or methylations, or proteins, peptides, polypeptides or RNA molecules, miRNAs, mRNAs, snoRNAs, hnRNAs or RNA that can be grouped so that information obtained about the set of bio-signatures in the group provides a sound basis for making a clinically relevant judgment such as a diagnosis, prognosis, or treatment choice. These sets of bio-signatures make up various portfolios of the invention. As with most diagnostic markers, it is often desirable to use the fewest number of markers sufficient to make a correct medical judgment. This prevents a delay in treatment pending further analysis as well inappropriate use of time and resources. Preferably, portfolios are established such that the combination of bio-signatures in the portfolio exhibit improved sensitivity and specificity relative to individual bio-signatures or randomly selected combinations of bio-signatures. In the context of the instant invention, the sensitivity of the portfolio can be reflected in the fold differences exhibited by a bio-signature's expression in the diseased state relative to the normal state. Specificity can be reflected in statistical measurements of the correlation of the signaling of gene expression, for example, with the condition of interest. In considering a group of markers in a bio-signature for inclusion in a portfolio, standard deviations, variances, co-variances, correlation coefficients, weighted averages, arithmetic sums, means, multiplicative values, weighted or balanced values or any mathematical manipulation of the values of 2 or more markers that can together be used to calculate a value or score that taken as a whole can be shown to produce greater sensitivity, specificity, negative predictive value, positive predictive value or accuracy can also be used in this capacity and are within the scope of this invention.

In another embodiment pattern recognition methods can be used. One example involves comparing biomarker expression profiles for various biomarkers (or bio-signature portfolios) to ascribe diagnoses. The expression profiles of each of the biomarker comprising the bio-signature portfolio are fixed in a medium such as a computer readable medium.

In one example, a table can be established into which the range of signals (e.g., intensity measurements) indicative of disease or physiological state is input. Actual patient data can then be compared to the values in the table to determine whether the patient samples are normal, benign, diseased, or represent a specific physiological state. In a more sophisticated embodiment, patterns of the expression signals (e.g., fluorescent intensity) are recorded digitally or graphically. In the example of RNA expression patterns from the biomarker portfolios used in conjunction with patient samples are then compared to the expression patterns. Pattern comparison software can then be used to determine whether the patient samples have a pattern indicative of the disease, a given prognosis, a pattern that indicates likeliness to respond to therapy, or a pattern that is indicative of a particular physiological state. The expression profiles of the samples are then compared to the portfolio of a control cell. If the sample expression patterns are consistent with the expression pattern(s) for disease, prognosis, or therapy-related response then (in the absence of countervailing medical considerations) the patient is diagnosed as meeting the conditions that relate to these various circumstances. If the sample expression patterns are consistent with the expression pattern derived from the normal/control exosome population then the patient is diagnosed negative for these conditions.

In another exemplary embodiment, a method for establishing biomarker expression portfolios is through the use of optimization algorithms such as the mean variance algorithm widely used in establishing stock portfolios. This method is described in detail in the U.S. Application Publication No. 20030194734, incorporated herein by reference. Alternatively, measured DNA alterations, changes in mRNA, protein, or metabolites to phenotypic readouts of efficacy and toxicity may be modeled and analyzed using algorithms, systems and methods described in U.S. Pat. Nos. 7,089,168, 7,415,359 and U.S. Application Publication Nos. 20080208784, 20040243354, or 20040088116, each of which is herein incorporated by reference in its entirety.

An exemplary process of bio-signature portfolio selection and characterization of an unknown is summarized as follows:

1. Choose baseline class.

2. Calculate mean, and standard deviation of each biomarker for baseline class samples.

3. Calculate (X*Standard Deviation+Mean) for each biomarker. This is the baseline reading from which all other samples will be compared. X is a stringency variable with higher values of X being more stringent than lower.

4. Calculate ratio between each Experimental sample versus baseline reading calculated in step 3.

5. Transform ratios such that ratios less than 1 are negative (eg. using Log base 10). (Under expressed biomarkers now correctly have negative values necessary for MV optimization).

6. These transformed ratios are used as inputs in place of the asset returns that are normally used in the software application.

7. The software will plot the efficient frontier and return an optimized portfolio at any point along the efficient frontier.

8. Choose a desired return or variance on the efficient frontier.

9. Calculate the Portfolio's Value for each sample by summing the multiples of each gene's intensity value by the weight generated by the portfolio selection algorithm.

10. Calculate a boundary value by adding the mean Bio-signature Portfolio Value for Baseline groups to the multiple of Y and the Standard Deviation of the Baseline's Bio-signature Portfolio Values. Values greater than this boundary value shall be classified as the Experimental Class.

11. Optionally one can reiterate this process until best prediction.

The process of selecting a bio-signature portfolio can also include the application of heuristic rules. Preferably, such rules are formulated based on biology and an understanding of the technology used to produce clinical results. More preferably, they are applied to output from the optimization method. For example, the mean variance method of bio-signature portfolio selection can be applied to microarray data for a number of biomarkers differentially expressed in subjects with a specific disease. Output from the method would be an optimized set of biomarkers that could include those that are expressed in exosomes as well as in diseased tissue. If samples used in the testing method are obtained from exosomes and certain biomarkers differentially expressed in instances of disease or physiological state could also be differentially expressed in exosomes, then a heuristic rule can be applied in which a bio-signature portfolio is selected from the efficient frontier excluding those that are differentially expressed in exosomes. Of course, the rule can be applied prior to the formation of the efficient frontier by, for example, applying the rule during data pre-selection.

Other statistical, mathematical and computational algorithms for the analysis of linear and non-linear feature subspaces, feature extraction and signal deconvolution in large scale datasets to identify exosome-derived multiplex analyte profiles for diagnosis, prognosis and therapy selection and/or characterization of define physiological states can be done using any combination of unsupervised analysis methods, including but not limited to: principal component analysis (PCA) and linear and non-linear independent component analysis (ICA); blind source separation, nongaussinity analysis, natural gradient maximum likelihood estimation; joint-approximate diagonalization; eigenmatrices; Gaussian radical basis function, kernel and polynominal kernel analysis sequential floating forward selection.

### Ex Vivo Harvesting of Exosomes

Exosomes for analysis and determination of a phenotype can also be from ex vivo harvesting. Cells can be cultured and that exosomes released from cells of interest in culture either result spontaneously or can be stimulated to release exosomes into the medium. (see for example, Zitvogel, et al. 1998. Nat. Med. 4: 594-600; Chaput, et al. 2004. J. Immunol. 172: 2137-214631: 2892-2900; Escudier, et al. 2005. J. Transl. Med. 3: 10; Morse, et al. 2005, J. Transl. Med. 3: 9; Peche, et al. 2006. Am. J. Transplant. 6: 1541-1550; Kim, et al. 2005. J. Immunol. 174: 6440-6448, all of which are herein incorporated by reference in their entireties). Cell lines or tissue samples can be grown to 80% confluence before being cultured in fresh DMEM for 72 h. Subsequent exosome production can be stimulated (see, for example, heat shock treatment of melanoma cells as described by Dressel, et al. 2003. Cancer Res. 63: 8212-8220, which is herein incorporated by reference in its entirety). The supernatant can then be harvested and exosomes prepared as described herein.

Exosomes produced ex vivo can, in one example, be cultured from a cell-of-origin or cell line of interest, exosomes can be isolated from the cell culture medium and subsequently labeled with a magnetic label, a fluorescent moiety, a radioisotope, an enzyme, a chemiluminescent probe, a metal particle, a non-metal colloidal particle, a polymeric dye particle, a pigment molecule, a pigment particle, an electrochemically active species, semiconductor nanocrystal or other nanoparticles including quantum dots or gold particles to be reintroduced in vivo as a label for imaging analysis. Ex vivo cultured exosomes can alternatively be used to identify novel bio-signatures by setting up culturing conditions for a given cell-of-origin with characteristics of interest, for example a culture of lung cancer cells or cell line with a known EGFR mutation that confers resistant to or susceptibility to gefitinib, then exposing the cell culture to gefitinib, isolating exosomes that arise from the culture and subsequently analyzing them on a discovery array to look for novel antigens or binding agents expressed on the outside of exosomes that could be used as a bio-signature to capture this species of exosome. Additionally, it would be possible to isolate any other biomarkers or bio-signatures found within these exosomes for discovery of novel signatures (including but not limited to nucleic acids, proteins, lipids, or combinations thereof) that may have clinical diagnostic, prognostic or therapy related implication.

Cells of interest can also be first isolated and cultured from tissues of interest. For example, human hair follicles in the growing phase, anagen, can be plucked individually from a patient's scalp using sterile equipment and plasticware, taking care not to damage the follicle. Each sample can be transferred to a Petri dish containing sterile PBS for tissue culture. Isolated human anagen hair follicles can be carefully transferred to an individual well of a 24-well plate containing 1 ml of William's E medium. Follicles can be maintained free-floating at 37° C. in an atmosphere of 5% CO₂ and 95% air in a humidified incubator. Medium can be changed every 3 days, taking care not to damage the follicles. Cells can then be collected and spun down from the media. Exosomes may then be isolated using antigens or cellular binding partners that are specific to such cell-of-origin specific exosomes using methods as previously described. Biomarkers and bio-signatures can then be isolated and characterized by methods known to those skilled in the art.

Cells of interest may also be cultured under microgravity or zero-gravity conditions or under a free-fall environment. For example, NASA's bioreactor technology will allow such cells to be grown at much faster rate and in much greater quantities. Exosomes may then be isolated using antigens or cellular binding partners that are specific to such cell-of-origin specific exosomes using methods as previously described.

Rotating wall vessels or RWVersus are a class of bioreactors developed by and for NASA that are designed to grow suspension cultures of cells in a quiescent environment that simulates microgravity can also be used. (see for exapmle, U.S. Pat. Nos. 5,026,650; 5,153,131; 5,153,133; 5,437,998; 5,665,594; 5,702,941; 7,351,584, 5,523,228, 5,104,802, 6,117,674, Schwarz, R P, et al., J. Tiss. Cult. Meth. 14:51-58, 1992; Martin et al., Trends in biotechnology 2004; 22; 80-86, Li et al., Biochemical Engineering Journal 2004; 18; 97-104, Ashammakhi et al., Journal Nanoscience Nanotechnology 2006; 9-10: 2693-2711, Zhang et al., International Journal of Medicine 2007; 4: 623-638, Cowger, N L, et al., Biotechnol. Bioeng 64:14-26, 1999, Spaulding, G F, et al., J. Cell. Biochem. 51:249-251, 1993, Goodwin, T J, et al., Proc. Soc. Exp. Biol. Med. 202:181-192, 1993; Freed, L E et al., In Vitro Cell. Dev. Biol. 33:381-385, 1997, Clejan, S. et al, Biotechnol. Bioeng. 50:587-597, 1996). Khaoustov, V I, et al., In Vitro Cell. Dev. Biol. 35:501-509. 1999, each of which is herein incorporated by reference in its entirety).

Alternatively, cells of interest or cell-of-origin specific exosomes that have been isolated may be cultured in a stationary phase plug-flow bioreactor as generally described in U.S. Pat. No. 6,911,201, and U.S. Application Publication Nos. 20050181504, 20050180958, 20050176143 and 20050176137, each of which is herein incorporated by reference in its entirety. Alternatively, cells of interest or cell-origin specific exosomes may also be isolated and cultured as generally described in U.S. Pat. No. 5,486,359.

One embodiment can include the steps of providing a tissue specimen containing cells of interest or cell-origin specific exosomes, adding cells or exosomes from the tissue specimen to a medium which allows, when cultured, for the selective adherence of only the cells of interest or cell-origin specific exosomes to a substrate surface, culturing the specimen-medium mixture, and removing the non-adherent matter from the substrate surface is generally described in U.S. Pat. No. 5,486,359, which is herein incorporated by reference in its entirety.

### Exosomes as Imaging Tools

In other embodiments, exosomes can be used as imaging tools. Labeled circulating tumor cells (CTCs) can be noninvasively visualized in vivo as they flow through the peripheral vasculature (He, W et al. (2007) PNAS 104(28)11760-11765). The method can involve i.v. injection of a tumor-specific fluorescent ligand followed by multiphoton fluorescence imaging of superficial blood vessels to quantitate the flowing CTCs. Studies in mice with metastatic tumors demonstrated that CTCs can be quantitated weeks before metastatic disease is detected by other means. Similar methods could be used and applied to circulating cell-of-origin specific exosomes as well. The decision to administer chemotherapy after tumor resection usually depends on an oncologist's assessment of the presence of microscopic metastatic disease. Although computed tomography, MRI, tissue/sentinel lymph node biopsy or serum cancer marker analysis can each detect some level of residual disease, the presence of circulating tumor derived exosomes can correlate most sensitively with cancer progression and metastasis. Noninvasive imaging of these exosomes in real time as they flow through the peripheral vasculature could improve detection sensitivity by enabling analysis of significantly larger blood volumes (potentially the entire blood volume of the patient). Exosomes isolated from bodily fluid, purified, labeled and then reintroduced into the system can be used for identification of early tumors not yet visible by traditional imaging methods (e.g. early breast tumors or early ovarian tumor cells). Labeled exosomes can also be used as a signal to identify tumors of metastatic potential.

In one embodiment, exosomes can be labeled by peptide/antigen targeting to label the exosomes either in vivo or in vitro and then reintroduce in the circulatory system for the purposes of diagnostic imaging. Suitable labels may include those that may be detected by intravital flow cytometry, X-radiography, NMR, PET/SPECT or MRI. For X-radiographic techniques, suitable labels include any radioisotope that emits detectable radiation but that is not overtly harmful to the patient, such as barium or cesium, for example. Suitable labels for NMR or MRI generally include those with a detectable characteristic spin, such as deuterium. Suitable imaging systems may be used to detect the labeled exosomes in the circulatory system.

The labeled exosomes can be administered by arterial or venous injection, and can be formulated as a sterile, pyrogen-free, parenterally acceptable aqueous solution. The preparation of such parenterally acceptable solutions, having due regard to pH, isotonicity, stability, and the like, is within the skill in the art. A preferred formulation for intravenous injection should contain, in addition to the labeled exosomes, an isotonic vehicle such as Sodium Chloride Injection, Ringer's Injection, Dextrose Injection, Dextrose and Sodium Chloride Injection, Lactated Ringer's Injection, or other vehicle. An effective amount of labeled exosomes can be an amount sufficient to yield an acceptable image using equipment which is available for clinical use. An effective amount of the labeled exosomes may be administered in more than one injection. Effective amounts of the labeled exosomes will vary according to factors such as the degree of susceptibility of the individual, the age, sex, and weight of the individual, idiosyncratic responses of the individual, the dosimetry. Effective amounts of the labeled exosomes will also vary according to instrument and film-related factors.

In a further embodiment, intravital flow cytometry can be used to noninvasively count labeled exosomes in vivo as they flow through the peripheral vasculature. The method can include i.v. injection of a tumor-specific fluorescent ligand followed by multiphoton fluorescence imaging of superficial blood vessels to quantitate the flowing exosomes. Intravital flow cytometry for detection of exosomes circumvents sampling limitations and renders quantitation of rare events statistically significant by enabling analysis of the majority of a patient's blood volume (≈5 liters).

Many human carcinomas overexpress a receptor for the vitamin folic acid (>90% of ovarian and endometrial cancers, 86% of kidney cancers, 78% of nonsmall cell lung cancers, etc). Alternatively, normal tissues either lack measurable folate receptors (FR) or express FR at a site that is inaccessible to parenterally administered drugs. Because FR-expressing cancer masses can be selectively labeled in vivo by injection of either radioactive or fluorescent folate conjugates that bind FR with nanomolar affinity it is possible for single exosomes to bind sufficient numbers of folate conjugates to allow their detection in vivo as they pass through a patient's peripheral vasculature. To increase signal-to-background ratios, a tumor-specific probe is used that rapidly clear circulation if left uncaptured by exosomes. For this purpose, folate-dye conjugates (e.g. folate-AlexaFluor 488) conjugates can be used because tumor-specific antibodies were found to promote phagocytic clearance of the exosomes to which they bound, thereby causing significant underestimation of exosomes counts. To further ensure that the cells labeled with folate-AlexaFluor 488 are indeed malignant, monoclonal anti-human antibodies can be used (e.g. CA125 for ovarian cancer) plus an appropriate secondary antibody conjugated to rhodamine-X.

In another embodiment, exosomes can be labeled in vivo by intravenously introducing a labeling agent that specifically targets the exosome for downstream imaging applications similar to those described above.

### RNAs

Assessment of any species of RNA can be used to characterize a disease or condition, such as cancer. The RNA can be microRNA (miRNA or miR), mRNA, small nuclear RNA, siRNA, small nucleolar RNA, or ribosomal RNA. The RNA pattern can comprise any RNA species, such as a microRNA (miRNA or miR), mRNA, small nuclear RNA, small nucleolar RNA, ribosomal RNA, or any combination thereof. The RNA pattern can comprise a single species of RNA or any combination of species, such as a miRNA and a mRNA. The assessment of an RNA can include determining or detecting the expression level of an RNA, such as the overexpression or underexpression as compared to a control, the absence or presence of an RNA, or the copy number of the RNA, such as copy numbers per microliter of sample, such as the copy number per microliter of plasma, or the copy number per microliter of serum. In some embodiments, assessing an RNA is detecting or determining the sequence of an RNA, or detecting a mutation or variant of an RNA.

A plurality of RNAs can be used to characterize a disease or condition, such as cancer. For example, an RNA pattern can comprise 2 or more different RNAs, such as at least 2, 3, 4, 5, 6, 7, 8, 9, 10, 15, 20, 25, 30, 40, 50, 60, 70, 80, 90, 100, 1000, 2500, 5000, 7500, 10,000, 100,000, 150,000, 200,000, 250,000, 300,000, 350,000, 400,000, 450,000, 500,000, 750,000, or 1,000,000 different RNAs. In some embodiments, the RNA pattern comprises at least 2, 3, 4, 5, 6, 7, 8, 9, 10, 15, 20, 25, 30, 40, 50, 60, 70, 80, 90, 100, 1000, 2500, 5000, 7500, 10,000, 100,000, 150,000, 200,000, 250,000, 300,000, 350,000, 400,000, 450,000, 500,000, 750,000, or 1,000,000 different miRNAs. The RNA pattern can also comprise one or more different miRNAs in combination with other species of RNAs, such as mRNA.

Also provided herein are methods of assessing one or more RNAs that can be used to diagnose a cancer. Diagnosis can include a negative diagnosis, such as no cancer is present. In other embodiments, diagnosis may include identifying the stage of a cancer, or the pre-symptomatic stages of a cancer. The one or more RNAs can also be used to provide a prognosis of a cancer, such as providing the risk or susceptibility of having a cancer or the aggressiveness or malignancy of a cancer.

Assessing one or more RNAs in sample can also be used to select a cancer therapy. Detection of one or more RNAs can be used to determine the efficacy of a cancer therapy or treatment, such as the relative improvement or deterioration of the subject's condition. Assessing one or more RNAs from samples of patients treated with effective therapies or non-effective therapies can be determined and used as a reference for selecting a therapy for a subject. In another embodiment, as a subject's cancer becomes progressively worse or better, the level of one or more RNAs may change, and compared to a reference of one or more RNAs from patients that were in a worse or better stage of the cancer.

The treatment or therapeutic selected based on one or more RNAs can be a treatment for cancer, such as an anti-cancer regimen or treatment that is selected from one or more of the following: vaccination, anti-growth factor or signal transduction therapy, radiotherapy, endocrine therapy, or human antibody therapy chemotherapy. The treatment can comprise a DNA damaging agent, topoisomerase inhibitor, mitotic inhibitor or a combination thereof. Many chemotherapeutics are presently known in the art and can be used in combination with the one or more compounds described herein. For example, the chemotherapeutic can be selected from the group consisting of: a mitotic inhibitor, alkylating agent, anti-metabolite, intercalating antibiotic, growth factor inhibitor, cell cycle inhibitor, enzyme, topoisomerase inhibitor, biological response modifier, anti-hormone, angiogenesis inhibitor, and anti-androgen. As used herein, cancer treatment, cancer therapy and the like encompasses treatments such as surgery, such as cutting, abrading, ablating (by physical or chemical means, or a combination of physical or chemical means), suturing, lasering or otherwise physically changing body tissues and organs), radiation therapy, administration of chemotherapeutic agents and combinations of any two or all of these methods. Combination treatments may occur sequentially or concurrently. Treatments, such as radiation therapy and/or chemotherapy, that are administered prior to surgery, are referred to as neoadjuvant therapy. Treatments, such as radiation therapy and/or chemotherapy, administered after surgery is referred to herein as adjuvant therapy. Examples of surgeries that may be used for prostate cancer treatment include, but are not limited to radical prostatectomy, cryotherapy, transurethral resection of the prostate, and the like.

Detection of one or more RNAs can also be used to determine the efficacy of a cancer therapy or treatment, such as the relative improvement or deterioration of the subject's condition. One or more RNAs for patients being treated for cancer can be determined and correlated to the improvement or beneficial efficacy, which is then used as a reference. For example, the improvement or beneficial efficacy can typically be assessed by determining if one or more of the following events has occurred: decreased or tumor size, decreased or tumor cell proliferation, decreased or numbers of cells, decreased or neovascularization and/or increased apoptosis. One or more of these occurrences may, in some cases, result in partial or total elimination of the cancer and prolongation of survival of the subject. Alternatively, for terminal stage cancers, treatment may result in stasis of disease, better quality of life and/or prolongation of survival. The converse result and/or stasis in any of those events can indicate inefficacy of treatment or therapy. Other methods of assessing treatment are known in the art and contemplated herein. Different assessments can be correlated with different RNAs or RNA patterns.

Assessing one or more RNAs can also be used monitor the progress of an anti-cancer treatment regimen or treatment in a subject, or the recurrence of a cancer. For example, the RNA or RNA patterns at various timepoint throughout a treatment. The RNA or RNA pattern can also be used to monitor a subject for the spread of a cancer. For example, miR-141 can be used for detecting the recurrence of colorectal cancer. Currently, colorectal cancer recurrence is measured by the level of the antigen CEA (carcino embryonic antigen). However, CEA can have confounding issues when used alone. For example, not all metastatic colorectal tumors express CEA, creating the need for additional markers, like miR-141. Similar issues are known for other single antigen tests for epithelial based cancers such as ovarian, breast, lung and bladder cancer.

Recurrence can be determined by periodically obtaining sample from a subject and monitoring the RNA or RNA pattern periodically from a sample of the subject. For example, an epithelial cancer has recurred if the miR-141 in the periodic blood samples shows a steady change in amount or is significantly elevated when compared to a miR-141 amount in a control sample that corresponds to subjects without epithelial cancer. In one embodiment, after a cancer is removed from a subject, for example surgically, the subject is monitored and through assessing an RNA or RNA pattern, the recurrence of the cancer at the same or secondary site can be identified so that additional therapies can be employed for treatment. In another embodiment, a subject is monitored during the treatment phase by having samples taken before and during treatment for analysis of an RNA or RNA pattern. Based on the RNA or RNA pattern, the therapy can be determined successful or not, if the therapy should be adapted or if the patient should try another therapy.

### Classification

In another embodiment, assessing one or more RNAs can be used to classify or stage a cancer. The classification and staging may also be used to assess treatment of cancers.

For example, the cancer can be classified based on the TNM classification of malignant tumors. This cancer staging system can be used to describe the extent of cancer in a subject's body. T describes the size of the tumor and whether it has invaded nearby tissue, N describes regional lymph nodes that are involved, and M describes distant metastasis. TNM is maintained by the International Union Against Cancer (UICC) and is used by the American Joint Committee on Cancer (AJCC) and the International Federation of Gynecology and Obstetrics (FIGO). One would understand that not all tumors have TNM classifications such as, for example, brain tumors. Generally, T (a,is,(0), 1-4) is measured as the size or direct extent of the primary tumor. N (0-3) refers to the degree of spread to regional lymph nodes: N0 means that tumor cells are absent from regional lymph nodes, N1 means that tumor cells spread to the closest or small numbers of regional lymph nodes, N2 means that tumor cells spread to an extent between N1 and N3; N3 means that tumor cells spread to most distant or numerous regional lymph nodes. M (0/1) refers to the presence of metastasis: M0 means that no distant metastasis are present; M1 means that metastasis has occurred to distant organs (beyond regional lymph nodes). Other parameters may also be assessed. G (1-4) refers to the grade of cancer cells (i.e., they are low grade if they appear similar to normal cells, and high grade if they appear poorly differentiated). R (0/1/2) refers to the completeness of an operation (i.e., resection-boundaries free of cancer cells or not). L (0/1) refers to invasion into lymphatic vessels. V (0/1) refers to invasion into vein. C (1-4) refers to a modifier of the certainty (quality) of V.

The methods also include classifying a prostate tumor based on the Gleason scoring system. The Gleason scoring system is based on microscopic tumor patterns assessed by a pathologist while interpreting the biopsy specimen. When prostate cancer is present in the biopsy, the Gleason score is based upon the degree of loss of the normal glandular tissue architecture (i.e. shape, size and differentiation of the glands). The classic Gleason scoring system has five basic tissue patterns that are technically referred to as tumor “grades.” The microscopic determination of this loss of normal glandular structure caused by the cancer is represented by a grade, a number ranging from 1 to 5, with 5 being the worst grade. Grade 1 is typically where the cancerous prostate closely resembles normal prostate tissue. The glands are small, well-formed, and closely packed. At Grade 2 the tissue still has well-formed glands, but they are larger and have more tissue between them, whereas at Grade 3 the tissue still has recognizable glands, but the cells are darker. At high magnification, some of these cells in a Grade 3 sample have left the glands and are beginning to invade the surrounding tissue. Grade 4 samples have tissue with few recognizable glands and many cells are invading the surrounding tissue. For Grade 5 samples, the tissue does not have recognizable glands, and are often sheets of cells throughout the surrounding tissue.

For example, after an initial analysis of a biological sample for one or more RNAs, based on the levels of one or more RNAs, a second analysis can be performed by a pathologist, where the pathologist determines a Gleason score for the sample. A biological fluid, such as urine can be analyzed for one or more RNAs prior to obtaining a biopsy to determine a Gleason score for a subject.

Assessing one or more RNAs can also be used to classify a cancer as malignant (e.g., aggressive) or benign (e.g., indolent). For example, a miRNA pattern can be determined for a biological sample and used to classify whether a cancer is aggressive or indolent. For example, the methods disclosed herein can be used to classify prostate cancer, by distinguishing between benign (e.g., indolent) and malignant (e.g., aggressive) prostate cancers.

Classification can be based on the amount or level of an RNA, or on the level of each of a plurality of RNAs. For example, the classification for a cancer is indolent epithelial cancer when the level of an RNAs, such as miRNA, is less than about 3000 copies per microliter of sample, for example, a serum sample. The classification for a cancer can be benign if the RNA level is between about 1000 and about 3000 copies per microliter, such as less than about 1100, 1200, 1300, 1400, 1500, 1600, 1700, 1800, 1900, 2000, 2100, 2200, 2300, 2400, 2500, 2600, 2700, 2800, 2900 or 3000. In some embodiments, a cancer is classified as benign when the expression level of a subset of RNAs that are detected is less than about 3000 copies per microliter of sample. In other embodiments, a cancer is classified as benign when the expression level of a subset of RNAs that are detected is between about 1000 and about 3000 copies per microliter of sample, such as less than about 1100, 1200, 1300, 1400, 1500, 1600, 1700, 1800, 1900, 2000, 2100, 2200, 2300, 2400, 2500, 2600, 2700, 2800, 2900 or 3000 copies per microliter.

In some embodiments, the classification of an epithelial cancer, such as prostate cancer, is malignant when the level of the RNA, such as miRNA, is at least about 9000, such as between about 9000 and about 26000 copies per microliter of sample, such as serum sample. For example, if the sample has at least about 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000, 12100, 12200, 12300, 12400, 12500, 13000, 13500, 14000, 14500, 15000, 15500, 16000, 16500, 17000, 17500, 18000, 18500, 19000, 19500, 20000, 20500, 21000, 21500, 22000, 22500, 23000, 23500, 24000, 24500, 25000, or 25,500 copies per microliter.

### Additional Biological Samples

The assessment of one or more RNAs can be performed on a sample obtained non-invasively, such as a urine sample or blood sample, to characterize a disease or condition, such as cancer. This can reduce the number of unnecessary biopsies or other invasive procedures for a subject. Thus, in some embodiments, assessing one or more RNAs is performed on a first sample from a subject. Based on the assessment of the one or more RNAs performed on the first sample, a second sample from the subject can be obtained for analysis to characterize a cancer. For example, the second sample can be of a different sample type from the first sample type and used for a different type of analysis, such as for histological examination, such as immunohistochemistry (IHC), in situ hybridization (such as fluorescent in situ hybridization), PCR, real-time PCR, microarray analysis or sequencing.

The first sample can be obtained in a less intrusive or less invasive method than is the second sample. For example, the first sample can be urine or blood, and the second sample can be a biopsy. For example, the first sample can be a blood sample that is used to assess one or more RNAs, and depending on the level of RNAs, a biopsy for histological examination can be obtained to characterize the cancer, such as diagnose the presence or absence of cancerous tissue or the stage of a cancer.

For example, if an RNA level in a first sample is between about 1500 to about 9000 copies per microliter, such as at least about 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, or 8900, a second sample, such as a biopsy or tissue sample for histological examination is taken from the subject. In some embodiments, if the level is between about 1500 to about 4500 copies per microliter, a second sample is taken from the subject. In yet other embodiments, a second sample is not obtained from a subject if the level of the RNA, such as a miRNA, is less than about 1500 copies per microliter, such as less than about 1100, 1200, 1300, 1400, or 1500.

In some embodiments, assessing one or more RNAs is used to determine the need for a second or third sample, such as a second or third biopsy. For example, after an initial elevated serum miR-141 is observed followed by a negative biopsy or a negative second biopsy. Such method includes the steps of obtaining a blood sample from a subject and determining an amount of miR-141 in serum of the subject's blood sample, and a biopsy is indicated when serum miR-141 is significantly different from a miR-141 amount in a control sample that corresponds to subjects without epithelial cancer, or to a previous determination of the same patient's miR-141 levels, any significant increase in miR-141 level indicating the need for another biopsy.

### Exosomes

In some embodiments, one or more RNAs disclosed herein are assessed from exosomes of a biological sample. Assessing one or more RNAs from an exosome can provide improved assay sensitivity and specificity for cancer detection, such as for the prognosis, monitoring, disease staging, and therapeutic decision-making of the cancer.

Assessing one or more RNAs to characterize a cancer can include detecting the amount of exosomes with a specific RNA or a specific RNA pattern. In other embodiments, detecting an RNA or RNA pattern of an exosome can be used to characterize a cancer. The exosome for analysis can be in a heterogeneous population of exosomes or a homogeneous, or substantially homogeneous, population of exosomes. The exosome can be purified or concentrated prior to analyzing the exosome. Various techniques that be used are disclosed herein. For example, exosomes may be concentrated or isolated from a biological sample using size exclusion chromatography, density gradient centrifugation, differential centrifugation, nanomembrane ultrafiltration, immunoabsorbent capture, affinity purification, microfluidic separation, or combinations thereof. For example, size exclusion chromatography such as gel permeation columns, centrifugation or density gradient centrifugation, and filtration methods can be used. For example, exosomes can be isolated by differential centrifugation, anion exchange and/or gel permeation chromatography (for example, as described in U.S. Pat. Nos. 6,899,863 and 6,812,023), sucrose density gradients, organelle electrophoresis (for example, as described in U.S. Pat. No. 7,198,923), magnetic activated cell sorting (MACS), or with a nanomembrane ultrafiltration concentrator. Various combinations of isolation or concentration methods can be used.

Binding agents, or capture agents, can be used to isolate exosomes by binding to exosomal components. A binding or capture agent may be used after the exosomes are concentrated or isolated from a biological sample. For example, exosomes are first isolated from a biological sample before exosomes with a specific biomarker are isolated using a binding agent for the biomarker. Thus, exosomes with the specific biomarker are isolated from a heterogeneous population of exosomes. Alternatively, a binding agent may be used on a biological sample comprising exosomes without a prior isolation step of exosomes. For example, a binding agent is used to isolate exosomes with a specific biomarker from a biological sample.

The binding agent can be, but not limited to, DNA, RNA, aptamers, monoclonal antibodies, polyclonal antibodies, Fabs, Fab′, single chain antibodies, synthetic antibodies, aptamers (DNA/RNA), peptoids, zDNA, peptide nucleic acids (PNAs), locked nucleic acids (LNAs), lectins, synthetic or naturally occurring chemical compounds (including but not limited to drugs, labeling reagents), or dendrimers.

Any of the exosomal proteins described herein can be used to capture and/or detect the various conditions described herein, including without limitation those in FIG. 1 or 3-60 or a target of a binding agent in FIG. 2. The exosomes can then be assessed for RNA content, e.g., microRNA content according to the methods of the invention.

In some embodiments, prostate specific exosomes, or prostatsomes, such as from a blood sample or urine is used for assessing one or more RNAs to characterize a cancer. Exosomes that are derived from a prostate cancer cells can be isolated using an antibody or aptamer, or any other binding agent, for one or more antigens that are specific for a cell of prostate cancer origin such as PSA, TMPRSS2, FASLG, TNFSF10, PSMA, NGEP, Il-7RI, CSCR4, CysLT1R, TRPM8, Kv1.3, TRPV6, TRPM8, PSGR, MISIIR, galectin-3, PCA3, TMPRSS2:ERG, fragments thereof, any combination thereof, or any combination of antigens that are specific for prostate cancer cells. The binding agent can be or be to PSA, PSMA, mAB 5D4, XPSM-A9, XPSM-A10, Galectin-3, E-selectin, Galectin-1, E4 (IgG2a kappa), or any combination thereof. The binding agent can also be to CD9, PSCA, TNFR, CD63, B7H3, MFG-E8, EpCam, Rab, CD81, STEAP, PCSA, PSMA, or 5T4, or any combination thereof. For example, the binding agent can comprise an antibody to at least one of CD63, CD9, CD81, B7H3, and EpCam. The binding agent or capture agent used to isolate an exosome can also be an agent that binds exosomal “housekeeping proteins,” such as CD63, CD9, CD81, or Rab-5b, or a binding agent for EpCAM is used to isolate exosomes.

### Sensitivity and Specificity

The methods and compositions disclosed herein can also provide increased sensitivity and the specificity for characterizing cancers, such as for detecting, diagnosing, prognosing, or monitoring for cancer recurrence and therapeutic efficacy are provided herein.

The sensitivity can be determined by: (number of true positives)/(number of true positives+number of false negatives). The specificity can be determined by: (number of true negatives)/(number of true negatives+number of false positives).

Assessing one or more RNAs disclosed herein can be used to characterize a cancer with at least about 70% or 75% specificity. For example, a cancer can be characterized with greater than about 76, 77, 78, 79, 80, 81, 82, 83, 84, 85, 86, 87, 88, 89, 90, 91, 92, 93, 94, 95, 96, or 97% specificity. The cancer can be characterized with at least about 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 998.2, 99.3, 99.4, 99.5, 99.6, 99.7, 99.8, 99.9% specificity. In yet other embodiments, the cancer can be characterized with 100% specificity.

In some embodiments, the cancer can be characterized with at least about 60% sensitivity, such as at least about 60, 65, 70, 75, 80, 81, 82, 83, 84, 85, 86, 87, 88, 89, 90, 91, 92, 93, 94, 95, 96, or 97% sensitivity. The cancer can be characterized with at least about 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9% sensitivity. In yet other embodiments, the cancer can be characterized with 100% sensitivity.

In some embodiments, assessing a plurality of RNAs provides increased specificity or sensitivity in the characterization of cancer as compared to assessing less than the plurality of RNAs. For example, the sensitivity or specificity may be at increased by at least about 5, 10, 15, 20, 30, 35, 40, 50, 75, 100, 150, 200, 250, 500, 1000% or more than detection with less than the plurality of RNAs. For example, the sensitivity for characterizing a cancer is 50% using one RNA, whereas using an additional RNA provides an increased sensitivity of 60%, an increase of 20%. Thus, in some embodiments, the number of RNAs analyzed is the number such that an increase in the number provides increased sensitivity or specificity. In some embodiments, assessing at least 2, 3, 4, 5, 6, 7, 8, 9, 10, 15, 20, 25, 30, 40, 50, 60, 70, 80, 90, 100, 1000, 2500, 5000, 7500, 10,000, 100,000, 150,000, 200,000, 250,000, 300,000, 350,000, 400,000, 450,000, 500,000, 750,000, or 1,000,000 RNAs provide increased specificity or sensitivity in the characterization of a cancer, as compared to less than the number of RNAs assessed. For example, assessing at least 2 RNAs, such as at least two miRNAs, can provide increased specificity or sensitivity in the characterization of cancer as compared to assessing one of the two miRNAs.

### MicroRNAs

The one or more RNAs assessed herein can comprise one or more microRNAs (miRNAs, miRs). MiRNAs are short RNA strands approximately 21-23 nucleotides in length. MiRNAs are encoded by genes that are transcribed from DNA but not translated into protein (non-coding RNA). Instead they are processed from primary transcripts known as pri-miRNA to short stem-loop structures called pre-miRNA and finally to functional miRNA, as the precursors typically form structures that fold back on each other in self-complementary regions. They are then processed by the nuclease Dicer in animals or DCL1 in plants. Mature miRNA molecules are partially complementary to one or more messenger RNA (mRNA) molecules. The sequences of miRNA can be accessed at publicly available databases, such as world wide web at microRNA.org or world wide web at mirz.unibas.ch/cgi/miRNA.cgi.

A number of miRNAs are involved in gene regulation, and miRNAs are part of a growing class of non-coding RNAs that is now recognized as a major tier of gene control. In some cases, miRNAs can interrupt translation by binding to regulatory sites embedded in the 3′-UTRs of their target mRNAs, leading to the repression of translation. Target recognition involves complementary base pairing of the target site with the miRNA's seed region (positions 2-8 at the miRNA's 5′ end), although the exact extent of seed complementarity is not precisely determined and can be modified by 3′ pairing. In other cases, miRNAs function like small interfering RNAs (siRNA) and bind to perfectly complementary mRNA sequences to destroy the target transcript.

Characterization of a number of miRNAs indicates that they influence a variety of processes, including early development, cell proliferation and cell death, apoptosis and fat metabolism. For example, some miRNAs, such as lin-4, let-7, mir-14, mir-23, and bantam, have been shown to play critical roles in cell differentiation and tissue development. Others are believed to have similarly important roles because of their differential spatial and temporal expression patterns.

In some embodiments, a single miRNA is assessed to characterize a cancer. In yet other embodiments, at least 2, 3, 4, 5, 6, 7, 8, 9, 10, 15, 20, 25, 30, 40, 50, 60, 70, 80, 90, 100, 1000, 2500, 5000, 7500, 10,000, 100,000, 150,000, 200,000, 250,000, 300,000, 350,000, 400,000, 450,000, 500,000, 750,000, or 1,000,000 miRNAs are assessed. In some embodiments, 1 or more miRNAs is assessed in combination with other species of RNAs, such as mRNA, to characterize a cancer.

In some embodiments, the miRNAs are used to detect prostate cancer. For example, the level of a microRNA that is detectable in sample can be indicative of prostate cancer and levels that are not detectable are not indicative of prostate cancer. In some embodiments, detection of 1, 2, 3, 4, 5, 6, 7, 8, 9, 10, 15, 20, 25, 30, 40, 50, 60, 70, 80, 90, 100, 1000, 2500, 5000, 7500, 10,000, 100,000, 150,000, 200,000, 250,000, 300,000, 350,000, 400,000, 450,000, 500,000, 750,000, 1,000,000 or more miRNAs is used to detect prostate cancer. A change in the expression level, such as absence, presence, underexpression or overexpression of the miRNA as compared to a reference level, such as a level determined for a subject without the cancer (such as age and sex controlled), can be used to characterize a cancer for the subject.

For example, a reference level for classifying a prostate cancer as benign or malignant can include obtaining a blood sample from a subject, determining an amount of a miRNA in the subject's blood sample, and comparing the amount of the miRNA to one or more controls having benign prostate cancer or malignant prostate cancer. The step of comparing the amount of the miRNA to one or more controls may include the steps of obtaining a range of the miRNA found in the blood for a plurality of subjects having benign prostate cancer to arrive at a first control range, obtaining a range of the miRNA found in the blood for a plurality of subjects having malignant prostate cancer to arrive at a second control range, and comparing the amount of the miRNA in the subject's blood sample with the first and second control ranges to determine if the subject's prostate cancer is classified as benign prostate cancer or malignant prostate cancer.

### MiR-200 Family

In some embodiments, the miRNA is a member of the miR-200 family. The miR-200 family is believed to determine the epithelial phenotype of cancer cells by targeting the E-cadherin repressors ZEB 1 and ZEB2. The miR-200 family includes miR-141, miR-236, miR-200a, mir-200b, mir-200c and mir-429. In some embodiments more than one miR-200 family member is analyzed to detect an epithelial cancer.

For example, miR-141 can be obtained from blood (serum or plasma) and correlated with the occurrence of metastatic epithelial cancer. MiR-141 can be used to detect cancer recurrence and therapeutic efficacy for epithelial based cancers, such as prostate cancer, including the use of miR-141 to monitor subjects who have undergone surgical removal of their cancer. For example, currently subjects are monitored with other markers like serum PSA for prostate cancer. A steady rise in serum PSA would indicate a recurrence and spread of the cancer. However, many prostate cancer metastases do not express PSA and are therefore missed by this monitoring method. By the time the cancer has been detected it has often spread beyond any treatment options. Other epithelial cancers have similar issues regarding current diagnostic regimens.

### Gene Associated MiRNAs

The miRNA can also be a miRNA that interacts with the mRNA of PFKFB3, RHAMM (HMMR), cDNA F1142103, ASPM, CENPF, NCAPG, Androgen Receptor, EGFR, HSP90, SPARC, DNMT3B, GART, MGMT, SSTR3, or TOP2B. For example, such as the microRNAs that can be detected, and the gene with which they are associated as listed in Table 2. The miRs can be used to characterize an epithelial cancer, such as prostate cancer.

TABLE 2
Gene Name and Their Associated miRNAs

| Gene | miRNA Associated with Gene |
|---|---|
| Androgen | hsa-miR-124a, hsa-miR-130a, hsa-miR-130b, hsa-miR-143, hsa-miR-149, hsa- |
| receptor | miR-194, hsa-miR-29b, hsa-miR-29c, hsa-miR-301, hsa-miR-30a-5p, hsa-miR- |
| | 30d, hsa-miR-30e-5p, hsa-miR-337, hsa-miR-342, hsa-miR-368, hsa-miR-488, |
| | hsa-miR-493-5p, hsa-miR-506, hsa-miR-512-5p, hsa-miR-644, hsa-miR-768-5p, |
| | hsa-miR-801 |
| DNMT3B | hsa-miR-618, hsa-miR-1253, hsa-miR-765, hsa-miR-561, hsa-miR-330-5p, hsa- |
| | miR-326, hsa-miR-188, hsa-miR-203, hsa-miR-221, hsa-miR-222, hsa-miR-26a, |
| | hsa-miR-26b, hsa-miR-29a, hsa-miR-29a, hsa-miR-29b, hsa-miR-29c, hsa-miR- |
| | 370, hsa-miR-379, hsa-miR-429, hsa-miR-519e*, hsa-miR-598, hsa-miR-618, |
| | hsa-miR-635 |
| GART | hsa-miR-101, hsa-miR-101, hsa-miR-141, hsa-miR-144, hsa-miR-182, hsa-miR- |
| | 189, hsa-miR-199a, hsa-miR-199b, hsa-miR-200a, hsa-miR-200b, hsa-miR-202, |
| | hsa-miR-203, hsa-miR-223, hsa-miR-329, hsa-miR-383, hsa-miR-429, hsa-miR- |
| | 433, hsa-miR-485-5p, hsa-miR-493-5p, hsa-miR-499, hsa-miR-519a, hsa-miR- |
| | 519b, hsa-miR-519c, hsa-miR-569, hsa-miR-591, hsa-miR-607, hsa-miR-627, |
| | hsa-miR-635, hsa-miR-659 |
| MGMT | hsa-miR-122a, hsa-miR-142-3p, hsa-miR-17-3p, hsa-miR-181a, hsa-miR-181b, |
| | hsa-miR-181c, hsa-miR-181d, hsa-miR-199b, hsa-miR-200a*, hsa-miR-217, hsa- |
| | miR-302b*, hsa-miR-32, hsa-miR-324-3p, hsa-miR-34a, hsa-miR-371, hsa-miR- |
| | 425-5p, hsa-miR-496, hsa-miR-514, hsa-miR-515-3p, hsa-miR-516-3p, hsa-miR- |
| | 574, hsa-miR-597, hsa-miR-603, hsa-miR-653, hsa-miR-655, hsa-miR-92, hsa- |
| | miR-92b, hsa-miR-99a |
| Top2B | hsa-miR-548f, hsa-miR-548a-3p, hsa-miR-548g, hsa-miR-513a-3p, hsa-miR- |
| | 548c-3p, hsa-miR-101, hsa-miR-653, hsa-miR-548d-3p, hsa-miR-575, hsa-miR- |
| | 297, hsa-miR-576-3p, hsa-miR-548b-3p, hsa-miR-624, hsa-miR-548n, hsa-miR- |
| | 758, hsa-miR-1253, hsa-miR-1324, hsa-miR-23b, hsa-miR-320a, hsa-miR-320b, |
| | hsa-miR-1183, hsa-miR-1244, hsa-miR-23a, hsa-miR-451, hsa-miR-568, hsa- |
| | miR-1276, hsa-miR-548e, hsa-miR-590-3p, hsa-miR-1, hsa-miR-101, hsa-miR- |
| | 126, hsa-miR-126*, hsa-miR-129, hsa-miR-136, hsa-miR-140, hsa-miR-141, hsa- |
| | miR-144, hsa-miR-147, hsa-miR-149, hsa-miR-18, hsa-miR-181b, hsa-miR-181c, |
| | hsa-miR-182, hsa-miR-184, hsa-miR-186, hsa-miR-189, hsa-miR-191, hsa-miR- |
| | 19a, hsa-miR-19b, hsa-miR-200a, hsa-miR-206, hsa-miR-210, hsa-miR-218, hsa- |
| | miR-223, hsa-miR-23a, hsa-miR-23b, hsa-miR-24, hsa-miR-27a, hsa-miR-302, |
| | hsa-miR-30a, hsa-miR-31, hsa-miR-320, hsa-miR-323, hsa-miR-362, hsa-miR- |
| | 374, hsa-miR-383, hsa-miR-409-3p, hsa-miR-451, hsa-miR-489, hsa-miR-493- |
| | 3p, hsa-miR-514, hsa-miR-542-3p, hsa-miR-544, hsa-miR-548a, hsa-miR-548b, |
| | hsa-miR-548c, hsa-miR-548d, hsa-miR-559, hsa-miR-568, hsa-miR-575, hsa- |
| | miR-579, hsa-miR-585, hsa-miR-591, hsa-miR-598, hsa-miR-613, hsa-miR-649, |
| | hsa-miR-651, hsa-miR-758, hsa-miR-768-3p, hsa-miR-9* |
| HSP90 | hsa-miR-1, hsa-miR-513a-3p, hsa-miR-548d-3p, hsa-miR-642, hsa-miR-206, hsa- |
| | miR-450b-3p, hsa-miR-152, hsa-miR-148a, hsa-miR-148b, hsa-miR-188-3p, hsa- |
| | miR-23a, hsa-miR-23b, hsa-miR-578, hsa-miR-653, hsa-miR-1206, hsa-miR-192, |
| | hsa-miR-215, hsa-miR-181b, hsa-miR-181d, hsa-miR-223, hsa-miR-613, hsa- |
| | miR-769-3p, hsa-miR-99a, hsa-miR-100, hsa-miR-454, hsa-miR-548n, hsa-miR- |
| | 640, hsa-miR-99b, hsa-miR-150, hsa-miR-181a, hsa-miR-181c, hsa-miR-522, |
| | hsa-miR-624, hsa-miR-1, hsa-miR-130a, hsa-miR-130b, hsa-miR-146, hsa-miR- |
| | 148a, hsa-miR-148b, hsa-miR-152, hsa-miR-181a, hsa-miR-181b, hsa-miR-181c, |
| | hsa-miR-204, hsa-miR-206, hsa-miR-211, hsa-miR-212, hsa-miR-215, hsa-miR- |
| | 223, hsa-miR-23a, hsa-miR-23b, hsa-miR-301, hsa-miR-31, hsa-miR-325, hsa- |
| | miR-363*, hsa-miR-566, hsa-miR-9, hsa-miR-99b |
| ASPM | hsa-miR-1, hsa-miR-122a, hsa-miR-135a, hsa-miR-135b, hsa-miR-137, hsa-miR- |
| | 153, hsa-miR-190, hsa-miR-206, hsa-miR-320, hsa-miR-380-3p, hsa-miR-382, |
| | hsa-miR-433, hsa-miR-453, hsa-miR-493-5p, hsa-miR-496, hsa-miR-499, hsa- |
| | miR-507, hsa-miR-517b, hsa-miR-548a, hsa-miR-548c, hsa-miR-567, hsa-miR- |
| | 568, hsa-miR-580, hsa-miR-602, hsa-miR-651, hsa-miR-653, hsa-miR-758, hsa- |
| | miR-9* |
| SPARC | hsa-miR-768-5p, hsa-miR-203, hsa-miR-196a, hsa-miR-569, hsa-miR-187, hsa- |
| | miR-641, hsa-miR-1275, hsa-miR-432, hsa-miR-622, hsa-miR-296-3p, hsa-miR- |
| | 646, hsa-miR-196b, hsa-miR-499-5p, hsa-miR-590-5p, hsa-miR-495, hsa-miR- |
| | 625, hsa-miR-1244, hsa-miR-512-5p, hsa-miR-1206, hsa-miR-1303, hsa-miR- |
| | 186, hsa-miR-302d, hsa-miR-494, hsa-miR-562, hsa-miR-573, hsa-miR-10a, hsa- |
| | miR-203, hsa-miR-204, hsa-miR-211, hsa-miR-29a, hsa-miR-29b, hsa-miR-29c, |
| | hsa-miR-29c, hsa-miR-339, hsa-miR-433, hsa-miR-452, hsa-miR-515-5p, hsa- |
| | miR-517a, hsa-miR-517b, hsa-miR-517c, hsa-miR-592, hsa-miR-96 |
| PFKB3 | hsa-miR-513a-3p, hsa-miR-1286, hsa-miR-488, hsa-miR-539, hsa-miR-658, hsa-miR- |
| | 524-5p, hsa-miR-1258, hsa-miR-150, hsa-miR-216b, hsa-miR-377, hsa-miR-135a, hsa- |
| | miR-26a, hsa-miR-548a-5p, hsa-miR-26b, hsa-miR-520d-5p, hsa-miR-224, hsa-miR- |
| | 1297, hsa-miR-1197, hsa-miR-182, hsa-miR-452, hsa-miR-509-3-5p, hsa-miR-548m, hsa- |
| | miR-625, hsa-miR-509-5p, hsa-miR-1266, hsa-miR-135b, hsa-miR-190b, hsa-miR-496, |
| | hsa-miR-616, hsa-miR-621, hsa-miR-650, hsa-miR-105, hsa-miR-19a, hsa-miR-346, hsa- |
| | miR-620, hsa-miR-637, hsa-miR-651, hsa-miR-1283, hsa-miR-590-3p, hsa-miR-942, hsa- |
| | miR-1185, hsa-miR-577, hsa-miR-602, hsa-miR-1305, hsa-miR-220c, hsa-miR-1270, hsa- |
| | miR-1282, hsa-miR-432, hsa-miR-491-5p, hsa-miR-548n, hsa-miR-765, hsa-miR-768-3p, |
| | hsa-miR-924 |
| HMMR | hsa-miR-936, hsa-miR-656, hsa-miR-105, hsa-miR-361-5p, hsa-miR-194, hsa-miR-374a, |
| | hsa-miR-590-3p, hsa-miR-186, hsa-miR-769-5p, hsa-miR-892a, hsa-miR-380, hsa-miR- |
| | 875-3p, hsa-miR-208a, hsa-miR-208b, hsa-miR-586, hsa-miR-125a-3p, hsa-miR-630, |
| | hsa-miR-374b, hsa-miR-411, hsa-miR-629, hsa-miR-1286, hsa-miR-1185, hsa-miR-16, |
| | hsa-miR-200b, hsa-miR-671-5p, hsa-miR-95, hsa-miR-421, hsa-miR-496, hsa-miR-633, |
| | hsa-miR-1243, hsa-miR-127-5p, hsa-miR-143, hsa-miR-15b, hsa-miR-200c, hsa-miR-24, |
| | hsa-miR-34c-3p |
| CENPF | hsa-miR-30c, hsa-miR-30b, hsa-miR-190, hsa-miR-508-3p, hsa-miR-384, hsa-miR-512- |
| | 5p, hsa-miR-548p, hsa-miR-297, hsa-miR-520f, hsa-miR-376a, hsa-miR-1184, hsa-miR- |
| | 577, hsa-miR-708, hsa-miR-205, hsa-miR-376b, hsa-miR-520g, hsa-miR-520h, hsa-miR- |
| | 519d, hsa-miR-596, hsa-miR-768-3p, hsa-miR-340, hsa-miR-620, hsa-miR-539, hsa-miR- |
| | 567, hsa-miR-671-5p, hsa-miR-1183, hsa-miR-129-3p, hsa-miR-636, hsa-miR-106a, hsa- |
| | miR-1301, hsa-miR-17, hsa-miR-20a, hsa-miR-570, hsa-miR-656, hsa-miR-1263, hsa- |
| | miR-1324, hsa-miR-142-5p, hsa-miR-28-5p, hsa-miR-302b, hsa-miR-452, hsa-miR-520d- |
| | 3p, hsa-miR-548o, hsa-miR-892b, hsa-miR-302d, hsa-miR-875-3p, hsa-miR-106b, hsa- |
| | miR-1266, hsa-miR-1323, hsa-miR-20b, hsa-miR-221, hsa-miR-520e, hsa-miR-664, hsa- |
| | miR-920, hsa-miR-922, hsa-miR-93, hsa-miR-1228, hsa-miR-1271, hsa-miR-30e, hsa- |
| | miR-483-3p, hsa-miR-509-3-5p, hsa-miR-515-3p, hsa-miR-519e, hsa-miR-520b, hsa- |
| | miR-520c-3p, hsa-miR-582-3p |
| NCAPG2 | hsa-miR-876-5p, hsa-miR-1260, hsa-miR-1246, hsa-miR-548c-3p, hsa-miR-1224-3p, hsa- |
| | miR-619, hsa-miR-605, hsa-miR-490-5p, hsa-miR-186, hsa-miR-448, hsa-miR-129-5p, |
| | hsa-miR-188-3p, hsa-miR-516b, hsa-miR-342-3p, hsa-miR-1270, hsa-miR-548k, hsa- |
| | miR-654-3p, hsa-miR-1290, hsa-miR-656, hsa-miR-34b, hsa-miR-520g, hsa-miR-1231, |
| | hsa-miR-1289, hsa-miR-1229, hsa-miR-23a, hsa-miR-23b, hsa-miR-616, hsa-miR-620 |
| EGFR | hsa-miR-105, hsa-miR-128a, hsa-miR-128b, hsa-miR-140, hsa-miR-141, hsa- |
| | miR-146a, hsa-miR-146b, hsa-miR-27a, hsa-miR-27b, hsa-miR-302a, hsa-miR- |
| | 302d, hsa-miR-370, hsa-miR-548c, hsa-miR-574, hsa-miR-587, hsa-miR-7 |
| SSTR3 | hsa-miR-125a, hsa-miR-125b, hsa-miR-133a, hsa-miR-133b, hsa-miR-136, hsa- |
| | miR-150, hsa-miR-21, hsa-miR-380-5p, hsa-miR-504, hsa-miR-550, hsa-miR- |
| | 671, hsa-miR-766, hsa-miR-767-3p |

Therefore, if one or more of the miRNAs in Table 2 appear in a concentration greater than 9000 copies per microliter of sample, such as a serum sample, the subject can be diagnosed with benign prostate cancer. If one or more of the miRNAs in Table 2 appear in a concentration less than 3000 copies per microliter of sample, the subject can be diagnosed with malignant prostate cancer. In some embodiments, if one or more of the miRNAs in Table 2 appear in a concentration between about 1000 to about 4500 copies per microliter of sample from a subject, a second biological sample from the subject is obtained. The second sample can be analyzed by histochemical analysis, such as by immunohistochemistry.

Furthermore, in various embodiments the micro RNAs associated with the genes for use in the methods and compositions of the invention (e.g., those overexpressed in prostate cancer) can be found in the micro RNA database online at world wide web at microrna.org; or microrna.sanger.ac.uk/sequences, or the predicted miRNAs queried at world wide web at diana.pcbi.upenn.edu/cgi-bin/miRGen/v3/.

The miRNA that interacts with PFKFB3 can be miR-513a-3p, miR-128, miR-488, miR-539, miR-658, miR-524-5p, miR-1258, miR-150, miR-216b, miR-377, miR-135a, miR-26a, miR-548a-5p, miR-26b, miR-520d-5p, miR-224, miR-1297, miR-1197, miR-182, miR-452, miR-509-3-5p, miR-548m, miR-625, miR-509-5p, miR-1266, miR-135b, miR-190b, miR-496, miR-616, miR-621, miR-650, miR-105, miR-19a, miR-346, miR-620, miR-637, miR-651, miR-1283, miR-590-3p, miR-942, miR-1185, miR-577, miR-602, miR-1305, miR-220c, miR-1270, miR-1282, miR-432, miR-491-5p, miR-548n, miR-765, miR-768-3p or miR-924. The one or more miRNA that interacts with PFKFB3 can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

The miRNA that interacts with RHAMM can be miR-936, miR-656, miR-105, miR-361-5p, miR-194, miR-374a, miR-590-3p, miR-186, miR-769-5p, miR-892a, miR-380, miR-875-3p, miR-208a, miR-208b, miR-586, miR-125a-3p, miR-630, miR-374b, miR-411, miR-629, miR-1286, miR-1185, miR-16, miR-200b, miR-671-5p, miR-95, miR-421, miR-496, miR-633, miR-1243, miR-127-5p, miR-143, miR-15b, miR-200c, miR-24 or miR-34c-3p. The one or more miRNA that interacts with RHAMM can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

The miRNA that interacts with CENPF can be miR-30c, miR-30b, miR-190, miR-508-3p, miR-384, miR-512-5p, miR-548p, miR-297, miR-520f, miR-376a, miR-1184, miR-577, miR-708, miR-205, miR-376b, miR-520g, miR-520h, miR-519d, miR-596, miR-768-3p, miR-340, miR-620, miR-539, miR-567, miR-671-5p, miR-1183, miR-129-3p, miR-636, miR-106a, miR-1301, miR-17, miR-20a, miR-570, miR-656, miR-1263, miR-1324, miR-142-5p, miR-28-5p, miR-302b, miR-452, miR-520d-3p, miR-548o, miR-892b, miR-302d, miR-875-3p, miR-106b, miR-1266, miR-1323, miR-20b, miR-221, miR-520e, miR-664, miR-920, miR-922, miR-93, miR-1228, miR-1271, miR-30e, miR-483-3p, miR-509-3-5p, miR-515-3p, miR-519e, miR-520b, miR-520c-3p or miR-582-3p. The one or more miRNA that interacts with CENPF can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

The miRNA that interacts with NCAPG can be miR-876-5p, miR-1260, miR-1246, miR-548c-3p, miR-1224-3p, miR-619, miR-605, miR-490-5p, miR-186, miR-448, miR-129-5p, miR-188-3p, miR-516b, miR-342-3p, miR-1270, miR-548k, miR-654-3p, miR-1290, miR-656, miR-34b, miR-520g, miR-123I, miR-1289, miR-1229, miR-23a, miR-23b, miR-616 or miR-620. The one or more miRNA that interacts with NCAPG can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

The miRNA that interacts with Androgen Receptor can be miR-124a, miR-130a, miR-130b, miR-143, miR-149, miR-194, miR-29b, miR-29c, miR-301, miR-30a-5p, miR-30d, miR-30e-5p, miR-337, miR-342, miR-368, miR-488, miR-493-5p, miR-506, miR-512-5p, miR-644, miR-768-5p or miR-801. The one or more miRNA that interacts with Androgen Receptor can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

The miRNA that interacts with EGFR can be miR-105, miR-128a, miR-128b, miR-140, miR-141, miR-146a, miR-146b, miR-27a, miR-27b, miR-302a, miR-302d, miR-370, miR-548c, miR-574, miR-587 or miR-7. The one or more miRNA that interacts with EGFR can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

The miRNA that interacts with HSP90 can be miR-1, miR-513a-3p, miR-548d-3p, miR-642, miR-206, miR-450b-3p, miR-152, miR-148a, miR-148b, miR-188-3p, miR-23a, miR-23b, miR-578, miR-653, miR-1206, miR-192, miR-215, miR-181b, miR-181d, miR-223, miR-613, miR-769-3p, miR-99a, miR-100, miR-454, miR-548n, miR-640, miR-99b, miR-150, miR-181a, miR-181c, miR-522, miR-624, miR-130a, miR-130b, miR-146, miR-148a, miR-148b, miR-152, miR-181a, miR-181b, miR-181c, miR-204, miR-206, miR-211, miR-212, miR-215, miR-223, miR-23a, miR-23b, miR-301, miR-31, miR-325, miR-363, miR-566, miR-9 or miR-99b. The one or more miRNA that interacts with HSP90 can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

The miRNA that interacts with SPARC can be miR-768-5p, miR-203, miR-196a, miR-569, miR-187, miR-641, miR-1275, miR-432, miR-622, miR-296-3p, miR-646, miR-196b, miR-499-5p, miR-590-5p, miR-495, miR-625, miR-1244, miR-512-5p, miR-1206, miR-1303, miR-186, miR-302d, miR-494, miR-562, miR-573, miR-10a, miR-203, miR-204, miR-211, miR-29, miR-29b, miR-29c, miR-339, miR-433, miR-452, miR-515-5p, miR-517a, miR-517b, miR-517c, miR-592 or miR-96. The one or more miRNA that interacts with SPARC can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

The miRNA that interacts with DNMT3B can be miR-618, miR-1253, miR-765, miR-561, miR-330-5p, miR-326, miR-188, miR-203, miR-221, miR-222, miR-26a, miR-26b, miR-29a, miR-29b, miR-29c, miR-370, miR-379, miR-429, miR-519e, miR-598, miR-618 or miR-635. The one or more miRNA that interacts with DNMT3B can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

The miRNA that interacts with GARTcan be miR-101, miR-141, miR-144, miR-182, miR-189, miR-199a, miR-199b, miR-200a, miR-200b, miR-202, miR-203, miR-223, miR-329, miR-383, miR-429, miR-433, miR-485-5p, miR-493-5p, miR-499, miR-519a, miR-519b, miR-519c, miR-569, miR-591, miR-607, miR-627, miR-635, miR-636 or miR-659. The one or more miRNA that interacts with GARTcan be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

The miRNA that interacts with MGMT can be miR-122a, miR-142-3p, miR-17-3p, miR-181a, miR-181b, miR-181c, miR-181d, miR-199b, miR-200a, miR-217, miR-302b, miR-32, miR-324-3p, miR-34a, miR-371, miR-425-5p, miR-496, miR-514, miR-515-3p, miR-516-3p, miR-574, miR-597, miR-603, miR-653, miR-655, miR-92, miR-92b or miR-99a. The one or more miRNA that interacts with MGMT can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

The miRNA that interacts with SSTR3 can be miR-125a, miR-125b, miR-133a, miR-133b, miR-136, miR-150, miR-21, miR-380-5p, miR-504, miR-550, miR-671, miR-766 or miR-767-3p. The one or more miRNA that interacts with SSTR3 can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

The miRNA that interacts with TOP2B can be miR-548f, miR-548a-3p, miR-548g, miR-513a-3p, miR-548c-3p, miR-101, miR-653, miR-548d-3p, miR-575, miR-297, miR-576-3p, miR-548b-3p, miR-624, miR-548n, miR-758, miR-1253, miR-1324, miR-23b, miR-320a, miR-320b, miR-1183, miR-1244, miR-23a, miR-451, miR-568, miR-1276, miR-548e, miR-590-3p, miR-1, miR-101, miR-126, miR-129, miR-136, miR-140, miR-141, miR-144, miR-147, miR-149, miR-18, miR-181b, miR-181c, miR-182, miR-184, miR-186, miR-189, miR-191, miR-19a, miR-19b, miR-200a, miR-206, miR-210, miR-218, miR-223, miR-23a, miR-23b, miR-24, miR-27a, miR-302, miR-30a, miR-31, miR-320, miR-323, miR-362, miR-374, miR-383, miR-409-3p, miR-451, miR-489, miR-493-3p, miR-514, miR-542-3p, miR-544, miR-548a, miR-548b, miR-548c, miR-548d, miR-559, miR-568, miR-575, miR-579, miR-585, miR-591, miR-598, miR-613, miR-649, miR-651, miR-758, miR-768-3p or miR-9. The one or more miRNA that interacts with TOP2B can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

In some embodiments, the one or more miRNA is selected from the group consisting of miR-498, miR-503miR-198, miR-302c, miR-345, miR-491-5p, miR-513, miR-26a-1/2, miR-375, miR-425, miR-194-1/2, miR-181a-1/2, let-7i, miR-25, miR-449, and miR-92-1/2. The one or more miRNAs can also be selected from the group consisting of: let-7a, let-7b, let-7c, let-7d, let-7g, miR-145, miR-195, miR-199, miR-497, let-7f, miR-22, miR-30_{—}5p, miR-490, miR-133a-1, miR-1-2, miR-218-2, miR-345, miR-410, miR-7-1/2, miR-145, miR-34a, miR-487, or let-7b. In other embodiments, the one or more miRNA is miR-99, miR-101, miR-130, miR-135, miR-141, miR-148, miR-182, miR-186, miR-206, miR-320, miR-374, miR-433, miR-496, miR-517, miR-590, miR-620, miR-768, miR-223, miR-203, miR-199, miR-519, miR-302, miR-30, miR-20, miR-200, miR-23, miR-29, miR-181, miR-548, and miR-370. The one or more miRNAs can be detected in a sample from a subject, such as determining the copy number per microliter of the one or more miRNA, and used to characterize a cancer. The copy number per microliter of miRNA can also be used to determine whether a second biological sample from a subject should be obtained for further analysis, such as by a pathologist.

In another embodiment, the one or more miRNA is miR-629, miR-671-3p, miR-9, miR-491, miR-182, miR125a-3p, miR-324-5p, miR-148b, miR-222, miR-141 or miR-370. The one or more miRNAs selected from the group consisting of: miR-629, miR-671-3p, miR-9, miR-491, miR-182, miR125a-3p, miR-324-5p, miR-148b, miR-222, and miR-141 can be used to characterize prostate cancer.

Furthermore, one or more miRNAs, such as those described in Table 2, can form a RNA patter with the mRNA of AR, PCA3, or any combination thereof, and used to characterize a cancer, such as prostate cancer. The RNA pattern can also comprise the snoRNA U50.

### Assessing RNA

RNA can be assessed using methods disclosed herein. Assessing the RNA may be qualitative or quantitative. Assessing RNA includes detecting the RNA, such as determining the expression level (such as overexpression or underexpression as compared to a control, the presence or absence of an RNA), determining the sequence of the RNA, determining any modifications of the RNA, or detecting any mutations or variations of the RNA. The RNA level may be determined to be present or absent, greater than or less than a control, or given a numerical value for the amount of RNA, such as the copies of RNA per microliter. The expression level of an RNA can be quantified, by absolute or relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

The RNA for assessment can be is isolated from a biological sample. The RNA can be isolated from exosomes of a biological sample, such as isolated exosomes using methods as described above.

The RNA can be isolated using kits for performing membrane based RNA purification, which are commercially available. Generally, kits are available for the small-scale (30 mg or less) preparation of RNA from cells and tissues (e.g. QIAGEN RNeasy Mini kit), for the medium scale (250 mg tissue) (e.g. QIAGEN RNeasy Midi kit), and for the large scale (1 g maximum) (QIAGEN RNeasy Maxi kit). Alternatively, RNA can be isolated using the method described in U.S. Pat. No. 7,267,950, or U.S. Pat. No. 7,267,950.

The RNA or nucleic acids derived from the RNA can be used for analysis. As used herein, a nucleic acid derived from an RNA refers to a nucleic acid for whose synthesis the RNA, a mRNA transcript, or a subsequence thereof has ultimately served as a template. Thus, a cDNA reverse transcribed from a transcript, an RNA transcribed from that cDNA, a DNA amplified from the cDNA, an RNA transcribed from the amplified DNA, and the like are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like.

The RNA can be detected by detecting one or more labels attached to the sample RNA. The labels may be incorporated by any of a number of means well known to those of skill in the art. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., 3H, 125I, 35S, 14C, or 32P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label. For example, miRNAs can be labeled and detect, such as using a radioactive phosphate at the 5′ end of the miRNA population can be used by using a polynucleotide kinase (Krichevsky A M, King K S, Donahue C P, Khrapko K, Kosik K S (2003) RNA 9: 1274-1281) or a radiolabeled, single nucleotide at the 3′ end using RNA ligase (see for example, U.S. Pat. No. 7,541,144). Commerically available kits can also be used to label the RNA. For example, miRNA can be labeled using kits from Ambion (e.g. mirVana™ labeling kit), Exiqon (e.g. miRCURY LNA microRNA Array Hy3™/Hy5™ Power Labeling kit), Integrated DNA Technologies (e.g. miRNA StarFire Nucleic Acid Labling) Minis Bio Corporation (e.g. LabelIT miRNA Labeling Kit) and others.

In one embodiment, after RNA has been isolated, to detect the RNA of interest, cDNA can be synthesized and either Taqman assays for specific mRNA targets can be performed according to manufacturer's protocol, or an expression microarray can be performed to look at highly multiplexed sets of expression markers in one experiment. Methods for establishing gene expression profiles include determining the amount of RNA that is produced by a gene that can code for a protein or peptide. This can be accomplished by reverse transcriptase PCR (RT-PCR), competitive RT-PCR, real time RT-PCR, differential display RT-PCR, quantitative RT-PCR, Northern Blot analysis and other related tests. These techniques can be performed using individual PCR reactions.

In some embodiments, complimentary DNA (cDNA) or complimentary RNA (cRNA) produced from mRNA is analyzed via microarray. The level of a miRNA gene product in a sample can be measured using any technique that is suitable for assessing RNA expression levels in a biological sample, including but not limited to Northern blot analysis, RT-PCR, in situ hybridization or microarray analysis. RNA detection can also be by hybridization with allele-specific probes, enzymatic mutation detection, ligation chain reaction (LCR), oligonucleotide ligation assay (OLA), flow-cytometric heteroduplex analysis, chemical cleavage of mismatches, mass spectrometry, nucleic acid sequencing, single strand conformation polymorphism (SSCP), denaturing gradient gel electrophoresis (DGGE), temperature gradient gel electrophoresis (TGGE), restriction fragment polymorphisms, serial analysis of gene expression (SAGE), or any combinations thereof.

If a quantitative result is desired, the methods disclosed herein typically use one or more controls for the relative frequencies of the amplified nucleic acids to achieve quantitative amplification. Methods of quantitative amplification are well known to those of skill in the art. For example, quantitative PCR involves simultaneously co-amplifying a known quantity of a control sequence using the same primers. This provides an internal standard that may be used to calibrate the PCR reaction. Other suitable amplification methods include, but are not limited to polymerase chain reaction (PCR) Innis, et al., PCR Protocols, A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990)), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87:1874 (1990)). Additional nucleic acid quantification methods known in the art include RT-PCR, Christmas-tree, ligase chain reaction, mass spectrometry, TMA, NASBA, branched chain reaction, and reverse transcriptase ligase chain reaction.

Additional detection and/or measurement methods include nucleic acid hybridization. Nucleic acid hybridization simply involves contacting a probe and target nucleic acid under conditions where the probe and its complementary target can form stable hybrid duplexes through complementary base pairing. As used herein, hybridization conditions refer to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety. Nucleic acids that do not form hybrid duplexes are washed away from the hybridized nucleic acids and the hybridized nucleic acids can then be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

Nucleic acid arrays can be used to detect the one or more RNAs of a sample. The production and application of high-density arrays in gene expression monitoring have been disclosed previously in, for example, WO 97/10365; WO 92/10588; WO95/35505; U.S. Pat. Nos. 6,040,138; 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637; and Hacia et al. (1996) Nature Genetics 14:441-447; Lockhart et al. (1996) Nature Biotechnol. 14:1675-1680; and De Risi et al. (1996) Nature Genetics 14:457-460.

In general, in an array, an oligonucleotide, or a cDNA, genomic DNA, or fragment thereof, of a known sequence occupies a known location on a substrate. A nucleic acid sample is hybridized with an array and the amount of nucleic acids hybridized to each probe in the array is quantified. One quantifying method is to use confocal microscope and fluorescent labels. Commercially available array platform systems, such as from Affymetrix (Santa Clara, Calif.), Agilent (Santa Clara, Calif.), Atlas™ (Clontech, Mountain View, Calif.), Exiqon (Denmark) and others can be used. One can use the knowledge of the genes described herein to design novel arrays of polynucleotides, cDNAs or genomic DNAs for screening methods described herein.

In yet other embodiments, the RNA can be detected using microspheres, particles, or bead-based platforms. For example, oligonucleotides that bind and detect the RNA can be conjugated to beads. In some embodiments, commercially available platforms, such as FlexmiR™ from Luminex (Austin, Tex.), or DASL assay from Illumina (San Diego, Calif.) can be used.

Furthermore, the methods can be performed using a microfluidic device. Such systems miniaturize and compartmentalize processes that allow for binding and detection of the target RNA. In some embodiments, the RNA is also isolated from a sample in a microfluidic device. Examples of microfluidic devices that may be used are described in U.S. Pat. Nos. 7,591,936, 7,581,429, 7,579,136, 7,575,722, 7,568,399, 7,552,741, 7,544,506, 7,541,578, 7,518,726, 7,488,596, 7,485,214, 7,467,928, 7,452,713, 7,452,509, 7,449,096, 7,431,887, 7,422,725, 7,422,669, 7,419,822, 7,419,639, 7,413,709, 7,411,184, 7,402,229, 7,390,463, 7,381,471, 7,357,864, 7,351,592, 7,351,380, 7,338,637, 7,329,391, 7,323,140, 7,261,824, 7,258,837, 7,253,003, 7,238,324, 7,238,255, 7,233,865, 7,229,538, 7,201,881, 7,195,986, 7,189,581, 7,189,580, 7,189,368, 7,141,978, 7,138,062, 7,135,147, 7,125,711, 7,118,910, and 7,118,661.

In some embodiments, multiplexing can be performed. For example, multiplexing can be performed using a particle-based assay, such as bead based assay, in combination with flow cytometry. Multiparametric immunoassays or other high throughput detection assays using bead coatings with cognate ligands and reporter molecules with specific activities consistent with high sensitivity automation can be used. For example, in a particle based assay system, a binding agent for an RNA of interest, such as an oligonucleotide, can be immobilized on addressable beads or microspheres. Each binding agent for each individual binding assay (such as an immunoassay when the binding agent is an antibody) can be coupled to a distinct type of microsphere (i.e., microbead) and the binding assay reaction takes place on the surface of the microspheres. Microspheres can be distinguished by different labels, for example, a microsphere with a specific binding agent would have a different signaling label as compared to another microsphere with a different binding agent. For example, microspheres can be dyed with discrete fluorescence intensities such that the fluorescence intensity of a microsphere with a specific binding agent is different than that of another microsphere with a different binding agent.

The methods of RNA detection can be used to determine the levels of RNA in a sample, such as the mean number of copies per microliter of serum. In some embodiments, the level of each of the RNAs is calculated with a 95% confidence interval about the mean (e.g., 15,648 of +/−10,431 copies per microliter). In other embodiments, the level of each of the RNAs is calculated with an 80, 81, 82, 83, 84, 85, 86, 87, 88, 89, 90, 91, 92, 93, or 94% confidence interval. In yet other embodiments, the level of each of the RNAs is calculated with a 95, 96, 97, 98, 99 or 100% confidence interval about the mean.

### RNA Patterns and PSA/PCA3 Levels

One or more RNAs can be assessed with one or more non-RNA biomarkers to characterize a cancer. A single sample can be used for assessing one or more RNAs, such as detecting one or more miRNAs, detecting one or more mRNAs, and detecting one or more non-RNA biomarkers. In some embodiments, more than one sample is used. For example, a single sample, such as blood or urine, can be used for detecting one or more miRNAs, PSA mRNA, PCA3 mRNA, and PSA protein.

A combination of an RNA level and a protein level can be used to characterize a cancer. In some embodiments, a combination of the expression level of a miRNA and a mRNA is used. For example, the mRNA level can be a of a gene or fusion gene, such as TMPRSS2:ERG or TMPRSS2:ETS. In other embodiments, the mRNA is of PCA or PCA3. In yet other embodiments, the expression levels of one or more miRNA, one or more mRNA, one or more proteins, or any combination thereof, is used to characterize a cancer. In yet other embodiments, the expression levels of one or more miRNA, one or more mRNA, one or more proteins, or any combination thereof, is determined for a first sample from a subject, such as urine or blood sample, and used to determine whether a second sample should be obtained from the subject for further analysis. For example, the second sample can be a biopsy.

For example, the expression level of one or more RNAs and of PSA protein can be used to characterize a prostate cancer. The expression level of one or more RNAs and of PSA protein can be determined in a first sample and used to determine whether a second sample, such as a biopsy, should be obtained for further analysis, such as for a histological examination. Assessing an RNA pattern and a PSA protein level can provide increased specificity or sensitivity in the characterization of prostate cancer, as compared to assessing the one or more RNAs alone or PSA protein levels alone. For example, the sensitivity, or specificity may be at least about 5, 10, 15, 20, 30, 35, 40, 50, 75, 100, 150, 200, 250, 500, 1000% or more than detection with the one or more RNAs alone or PSA protein level alone.

In some embodiments, a PCA3 level is used to characterize prostate cancer or determine whether a second sample, such as a biopsy, should be obtained for analysis. For example, in some embodiments, a miRNA level and a PCA3 mRNA level are used. Assessing a miRNA level and PCA3 mRNA level can provide increased specificity or sensitivity in the characterization of prostate cancer, as compared to assessing the miRNA level alone or the PCA3 mRNA level alone. For example, the sensitivity or specificity may be at least about 5, 10, 15, 20, 30, 35, 40, 50, 75, 100, 150, 200, 250, 500, 1000% or more.

In yet other embodiments, a miRNA level, PCA3 mRNA level, and PSA mRNA level are used to characterize prostate cancer or determine whether a second sample, such as a biopsy, should be obtained for analysis. Assessing a miRNA level, PCA3 mRNA level, and PSA mRNA levels can provide increased specificity or sensitivity in the characterization of prostate cancer, as compared to assessing 1 or 2 of the following: miRNA level, PCA3 mRNA level, and PSA mRNA level. For example, the sensitivity or specificity may be at least about 5, 10, 15, 20, 30, 35, 40, 50, 75, 100, 150, 200, 250, 500, 1000% or more.

In yet other embodiments, a miRNA level, PCA3 mRNA level, PSA mRNA level, and PSA protein level are used to characterize prostate cancer or determine whether a second sample, such as a biopsy, should be obtained for analysis. Assessing a miRNA level, PCA3 mRNA level, and PSA mRNA level can provide increased specificity or sensitivity in the characterization of a prostate cancer, as compared to assessing 1, 2, or 3 of the following: miRNA level, PCA3 mRNA level, PSA mRNA level, and PSA protein level. For example, the sensitivity or specificity may be at least about 5, 10, 15, 20, 30, 35, 40, 50, 75, 100, 150, 200, 250, 500, 1000% or more.

In some embodiments, the PCA3 mRNA level and PSA mRNA level are used to create a PCA3 score, which is a ratio of PCA3 mRNA level to PSA mRNA level, such as PCA3 mRNA copy number compared to PSA mRNA copy numbers. The PCA3 score can be used to characterize a prostate cancer or determine whether a second sample, such as a biopsy, should be obtained for analysis.

In some embodiments, the PCA3 score is used with the expression level of one or more RNAs, such as the level of a miRNA, to characterize a prostate cancer or determine whether a second sample, such as a biopsy, should be obtained for analysis. Assessing an RNA pattern and PCA3 score can provide increased specificity or sensitivity in the characterization of prostate cancer, as compared to assessing the one or more RNAs alone or PCA3 score alone. For example, the sensitivity, or specificity may be at least about 5, 10, 15, 20, 30, 35, 40, 50, 75, 100, 150, 200, 250, 500, 1000% or more.

In yet other embodiments, the PCA3 score is used with the expression level of one or more RNA and PSA protein to characterize a prostate cancer or determine whether a second sample, such as a biopsy, should be obtained for analysis. Assessing one or more RNAs and PSA protein and determining a PCA3 score can provide increased specificity or sensitivity in the characterization of prostate cancer, as compared to assessing 1 or 2 of the following: an RNA pattern, PSA protein level, and PCA3 score. For example, the sensitivity, or specificity may be at least about 5, 10, 15, 20, 30, 35, 40, 50, 75, 100, 150, 200, 250, 500, 1000% or more.

In yet other embodiments, prostate cancer is characterized by determining a product value by multiplying the level of an RNA with the level of PSA. The product value can then be used to characterize a prostate cancer. The product value can be used to diagnose a subject, to classify a cancer as benign or malignant, or to select a therapy for the subject. The product value for a subject can be compared to a reference value to characterize the cancer. For example, a reference value can be determined for diagnosing prostate cancer by determining the product value for patients with prostate cancer. Reference values can also be determined for different stages or prostate cancer, or for benign prostate cancer or malignant prostate cancer. Reference values can also be determined for drug efficacy, such as by determining reference values based on patients on effective prostate cancer therapeutics.

The product value can be used to characterize a prostate cancer with at least about 70% or 75% specificity. For example, a prostate cancer can be characterized using a product value with greater than about 76, 77, 78, 79, 80, 81, 82, 83, 84, 85, 86, 87, 88, 89, 90, 91, 92, 93, 94, 95, 96, or 97% specificity. The prostate cancer can be characterized with at least about 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 998.2, 99.3, 99.4, 99.5, 99.6, 99.7, 99.8, 99.9% specificity. In yet other embodiments, the cancer can be characterized with 100% specificity.

In some embodiments, the cancer can be characterized using a product value with at least about 60% sensitivity, such as at least about 60, 65, 70, 75, 80, 81, 82, 83, 84, 85, 86, 87, 88, 89, 90, 91, 92, 93, 94, 95, 96, or 97% sensitivity. The cancer can be characterized with at least about 97.1, 97.2, 97.3, 97.4, 97.5, 97.6, 97.7, 97.8, 97.8, 97.9, 98.0, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9% sensitivity. In yet other embodiments, the cancer can be characterized with 100% sensitivity. Furthermore, the product value can be used to characterize a prostate cancer with 100% specificity and 100% sensitivity. For example, a diagnosis of prostate cancer can be provided with 100% specificity and 100% sensitivity.

The level of RNA can be the number of copies of the miRNA per microliter of a sample and the level of PSA can be the amount of protein per microliter of sample, such as ng/ml. The amount of miRNA multiplied by the amount of PSA protein in a sample can be used to determine a product value for normal subjects and for subjects with prostate cancer. Thus, reference levels can be determined for normal subjects and for subjects with prostate cancer. The product value for a sample obtained from a subject can be determined and compared to the reference levels to characterize a cancer for the subject, such as provide a diagnosis. For example, a product value can be determined by multiplying the copies per microliter of miR-141 in a serum sample by the nanogram per microliters of PSA in a serum sample (see for example, FIG. 5). If the product value is less than 1500, 1550, 1400, 1450, or 1400, a diagnosis that the subject does not have prostate cancer can be provided. Alternatively, if the product value is greater than 1500, 1600, 1700, 1800, 1900 or 2000, a diagnosis that the subject has prostate cancer can be provided. In some embodiments, if the product value is greater than about 2000, 2100, 2200, or 2300, a diagnosis that the subject has prostate cancer is provided. A prostate cancer can be classified as benign if the product value is less than 1500. Alternatively, if the product value is greater than 1500, the cancer can be classified as malignant.

The product value can be used to classify the prostate cancer or determine whether a second sample, such as a biopsy should be obtained, for analysis. For example, if the product value is less than 1500, 1200, or 1000, a biopsy would not be obtained. In other embodiments, if the product value was greater than 1500, 1700, 1800, or 2000, a biopsy would be obtained.

In another embodiment, a method to classify a prostate cancer as benign or malignant as well as to determine whether a second sample should be obtained. For example, when the PSA protein is less than about 3 ng/mL, such as at least 2.9, 2.8, 2.7, 2.6, 2.5, 2.4, 2.3, 2.2, 2.1, or 2.0 ng/mL, the miRNA is less than about 3000 copies per microliter, such as less than about 2500, 2000, 1500, 1000 or 500, and optionally, the PCA3 score is less than 35, such as less than 30, 25, or 20, the prostate cancer is classified as benign, a second sample, such as biopsy, is not obtained, or both.

In another embodiment, when the miRNA is less than about 3000 copies per microliter, such as less than about 2500, 2000, 1500, 1000 or 500, and the PCA3 score is less than 35, such as less than 30, 25, or 20, the prostate cancer is classified as benign, a second sample, such as biopsy, is not obtained, or both.

When the PSA protein is greater than about 4 ng/mL, such as at least 4.1, 4.2, 4.3, 4.4, 4.5, 4.6, 4.7, 4.8, 4.9 or 5.0 ng/mL, the miRNA is greater than about 9000 copies per microliter, such as greater than about 9500, 10,000, 15,000 or 20,000, and optionally, the PCA3 score is greater than 35, such as at least 40, 45, or 50, the prostate cancer is classified as malignant, a second sample, such as biopsy, is obtained, or both.

In another embodiment, when the miRNA is greater than about 9000 copies per microliter, such as greater than about 9500, 10,000, 15,000 or 20,000, and the PCA3 score is greater than 35, such as at least 40, 45, or 50, the prostate cancer is classified as malignant, a second sample, such as biopsy, is obtained, or both.

### RNA Detection System and Kits

Also provided is a detection system configured to determine one or more RNAs for characterizing a cancer. For example, the detection system can be configured to assess at least 2, 3, 4, 5, 6, 7, 8, 9, 10, 15, 20, 25, 30, 40, 50, 60, 70, 80, 90, 100, 1000, 2500, 5000, 7500, 10,000, 100,000, 150,000, 200,000, 250,000, 300,000, 350,000, 400,000, 450,000, 500,000, 750,000, or 1,000,000 RNAs. For example, the detection system can be configured to assess 2, 3, 4, 5, 6, 7, 8, 9, 10, 15, 20, 25, 30, 40, 50, 60, 70, 80, 90, 100, 1000, 2500, 5000, 7500, 10,000, 100,000, 150,000, 200,000, 250,000, 300,000, 350,000, 400,000, 450,000, 500,000, 750,000, 1,000,000 or more miRNAs, wherein one or more of the miRNAs are selected from Table 2. In some embodiments, the one or more miRNAs detected by the system are selected from the group consisting of: miR-629, miR-671-3p, miR-9, miR-491, miR-182, miR125a-3p, miR-324-5p, miR-148b, miR-222, miR-141. In yet other embodiments, the one or more miRNAs are selected from the group consisting of miR-99, miR-101, miR-130, miR-135, miR-141, miR-148, miR-182, miR-186, miR-206, miR-320, miR-374, miR-433, miR-496, miR-517, miR-590, miR-620, miR-768, miR-223, miR-203, miR-199, miR-519, miR-302, miR-30, miR-20, miR-200, miR-23, miR-29, miR-181, miR-548 or miR-370. The detection system can also be configured to detect the mRNA levels of PSA, PCA or both.

The detection system can be a low density detection system or a high density detection system. For example, a low density detection system can detect up to about 100, 200, 300, 400, 500, or 1000 RNA, whereas a high density detection system can detect at least about 2000, 3000, 4000, 5000, 6000, 7000, 8000, 9,000, 10,000, 15,000, 20,000, 25,000, 50,000, or 100,000 RNAs. The detection system can be specific for detecting a species of RNA, such as miRNAs. A low density detection system for miRNA can detect up to about 100, 200, 300, 400, 500, or 1000 miRNAs. A high density detection system for miRNA can detect at least about 2000, 3000, 4000, 5000, 6000, 7000, 8000, 9,000, 10,000, 15,000, 20,000, 25,000, 50,000, or 100,000 miRNAs.

The detection system can comprise a set of probes that selectively hybridizes to the one or more of the RNAs. For example, the detection system can comprise a set of probes that selectively hybridizes to at least 2, 3, 4, 5, 6, 7, 8, 9, 10, 15, 20, 25, 30, 40, 50, 60, 70, 80, 90, 100, 1000, 2500, 5000, 7500, 10,000, 100,000, 150,000, 200,000, 250,000, 300,000, 350,000, 400,000, 450,000, 500,000, 750,000, or 1,000,000 miRNAs. For example, the set of probes can selectively hybridize to or more miRNAs selected from Table 2, one or more miRNAs are selected from the group consisting of: miR-629, miR-671-3p, miR-9, miR-491, miR-182, miR125a-3p, miR-324-5p, miR-148b, miR-222, miR-141. In yet other embodiments, the one or more miRNAs are selected from the group consisting of miR-99, miR-101, miR-130, miR-135, miR-141, miR-148, miR-182, miR-186, miR-206, miR-320, miR-374, miR-433, miR-496, miR-517, miR-590, miR-620, miR-768, miR-223, miR-203, miR-199, miR-519, miR-302, miR-30, miR-20, miR-200, miR-23, miR-29, miR-181, miR-548 or miR-370. The detection system can also comprise probes for detecting the mRNA levels of PSA, PCA or both.

The detection system can be a low density detection system or a high density detection system comprising probes to detect the RNAs. For example, a low density detection system can comprise probes to detect up to about 100, 200, 300, 400, 500, or 1000 RNA, whereas a high density detection system can comprise probes to detect at least about 2000, 3000, 4000, 5000, 6000, 7000, 8000, 9,000, 10,000, 15,000, 20,000, 25,000, 50,000, or 100,000 RNAs. The probes can be specific for detecting a species of RNA, such as miRNAs, such that a a low density detection system for miRNA can comprise probes for detecting up to about 100, 200, 300, 400, 500, or 1000 miRNAs. A high density detection system for miRNA can comprise probes for detecting at least about 2000, 3000, 4000, 5000, 6000, 7000, 8000, 9,000, 10,000, 15,000, 20,000, 25,000, 50,000, or 100,000 miRNAs.

The probes may be attached to a solid substrate, such as an array or bead. Alternatively, the probes are not attached. The detection system may be an array based system, a sequencing system, a PCR-based system, or a bead-based system, such as described above. The detection system may be part of a kit. Alternatively, the kit may comprise the one or more probe sets described herein. For example, the kit may comprise probes for detecting one or more of the miRNAs selected from the group consisting of: miR-629, miR-671-3p, miR-9, miR-491, miR-182, miR125a-3p, miR-324-5p, miR-148b, miR-222, or miR-141. In yet other embodiments, the one or more miRNAs are selected from the group consisting of: miR-99, miR101, miR-130, miR-135, miR-141, miR-148, miR-182, miR-186, miR-206, miR-320, miR-374, miR-433, miR-496, miR-517, miR-590, miR-620, miR-768, miR-223, miR-203, miR-199, miR-519, miR-302, miR-30, miR-20, miR-200, miR-23, miR-29, miR-181, miR-548 or miR-370. In some embodiments, the kit further comprises one or more reagents that selectively binds to PSA or PCA3. For example, the kit may comprise a reagent, such as a probe, to detect PSA protein levels or PSA mRNA levels. The kit may also comprise a reagent to detect PCA3 mRNA levels.

### Computer Systems

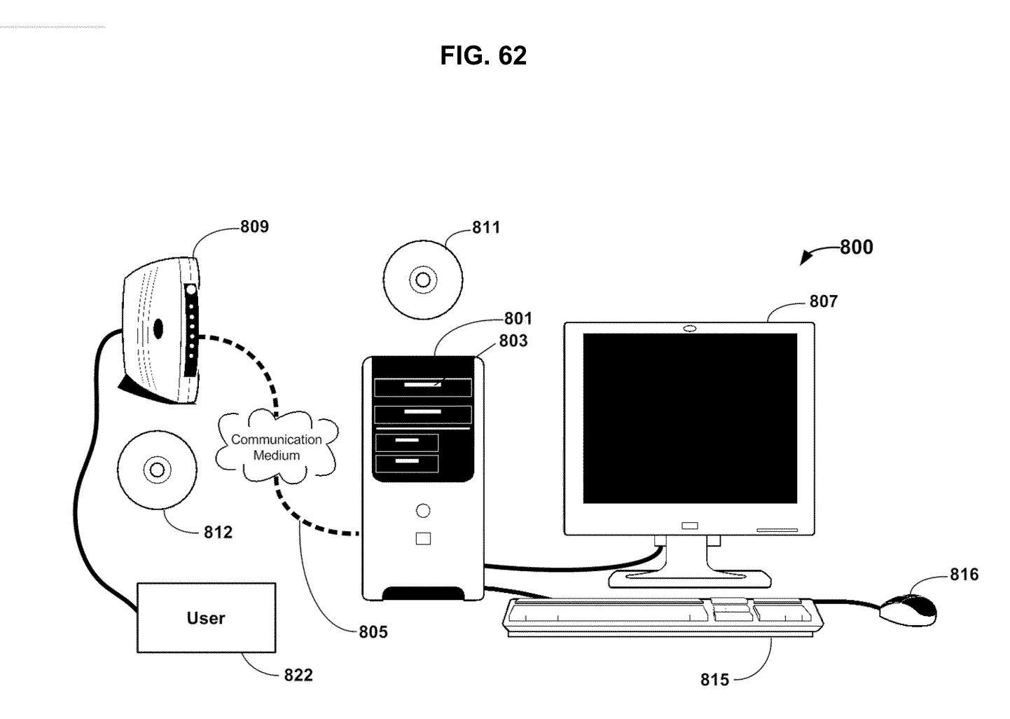

An exosome and/or RNA such as microRNA can be assayed for molecular features, for example, by determining an amount, presence or absence of one or more biomarkers such as listed FIGS. 1, 3-60 or Table 2. The data generated can be used to produce a bio-signature, which can be stored and analyzed by a computer system, such as shown in FIG. 62. The assaying or correlating of the bio-signature with one or more phenotypes can also be performed by computer systems, such as by using computer executable logic.

A computer system, such as shown in FIG. 62, can be used to transmit data and results following analysis. Accordingly, FIG. 62 is a block diagram showing a representative example logic device through which results from exosome analysis can be reported or generated. FIG. 62 shows a computer system (or digital device) 800 to receive and store data generated from exosome analysis, analyze the data to generate one or more bio-signatures, and produce a report of the one or more bio-signatures. The computer system can also perform comparisons and analyses of bio-signatures generated, and transmit the results. Alternatively, the computer system can receive raw data of exosome analysis, such as through transmission of the data over a network, and perform the analysis.

The computer system 800 may be understood as a logical apparatus that can read instructions from media 811 and/or network port 805, which can optionally be connected to server 809 having fixed media 812. The system shown in FIG. 62 includes CPU 801, disk drives 803, optional input devices such as keyboard 815 and/or mouse 816 and optional monitor 807. Data communication can be achieved through the indicated communication medium to a server 809 at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present invention can be transmitted over such networks or connections for reception and/or review by a party 822. The receiving party 822 can be but is not limited to an individual, a health care provider or a health care manager. In one embodiment, a computer-readable medium includes a medium suitable for transmission of a result of an analysis of a biological sample, such as bio-signatures determined by assessing exosomes and/or RNA such as microRNA according to the methods of the invention. The medium can include a result regarding an bio-signature of a subject, wherein such a result is derived using the methods described herein.

### Reimbursement Codes

In some embodiments, the use of exosomes and/or RNA such as microRNA as diagnostic, therapy-related or prognostic markers in the identification of disease, disease stage, progression or therapy can be assigned specific U.S. Medicare reimbursement codes. In one embodiment, the isolation and the use of cell-of-origin specific exosomes are used. The reimbursement code may be a code developed under the National Council for Prescription Drug Programs Professional Pharmacy Services (NCPDP/PPS code). A reimbursement code can be a diagnosis code utilized or recognized by an insurance company, for example. The diagnostic code is assignable based upon a reimbursement requirement by a third party. Alternatively, the diagnostic code is assignable based upon a need to analyze the utilization of medical resources. A set of diagnosis codes can conform to and/or be compatible with, for example, ICD (International Classification of Diseases) codes, 9th Edition, Clinical Modification, (ICD-9-CM), Volumes 1, 2 and 3; ICD-10, which is maintained and distributed by the U.S. Health and Human Services department; HCPCS (Health Care Financing Administration Common Procedure Coding System); NDC (National Drug Codes); CPT-4 (Current Procedural Terminology); Fourth Edition CDPN (Code on Dental Procedures and Nomenclature); SNOMED-RT “Systematicized Nomenclature of Medicine, Reference Terminology” by the College of American Pathologists; UMLS (Unified Medical Language System), by the National Library of Medicine; LOINC Logical Observation Identifiers, Names, and Codes; Regenstrief Institute and the Logical Observation Identifiers Names and Codes (LOINC®) Committee; Clinical Terms also known as “Read Codes”; DIN Drug Identification Numbers; Reimbursement Classifications including DRGs (Diagnosis Related Groups); CDT Current Dental Terminology; NIC (Nursing intervention codes); or Commercial Vocabulary Services (such as HealthLanguage by HealthLanguage Inc.), each of which is incorporated by reference in its entirety.

In one embodiment, each of the isolation methods for exosomes described herein can be assigned a specific reimbursement code. For example, each of the isolation methods of cell-of-origin specific exosomes described herein can be assigned a specific reimbursement code. In another embodiment, the specific bio-signature(s) obtained from the analysis of exosomes can be assigned a specific reimbursement code. In yet another embodiment, the specific bio-signature(s) obtained from the analysis of cell-of-origin specific exosomes can be assigned a specific reimbursement code. Alternatively, kits for the detection of a particular bio-signature of exosomes in a biological sample can be assigned to a specific reimbursement code. Alternatively, kits for the detection of a particular bio-signature of specific cell-of-origin exosomes in a biological sample can be assigned to a specific reimbursement code.

Similarly, various steps in methods of isolating and/or detecting RNAs such as microRNAs, including such RNAs within exosomes, can be assigned a specific reimbursement code.

### EXAMPLES

### Example 1

### Purification of Exosomes from Prostate Cancer Cell Lines

Prostate cancer cell lines are cultured for 3-4 days in culture media containing 20% FBS (fetal bovine serum) and 1% P/S/G. The cells are then pre-spun for 10 minutes at 400×g at 4° C. The supernatant is kept and centrifuged for 20 minutes at 2000×g at 4. The supernatant containing exosomes can be concentrated using a Millipore Centricon Plus-70 (Cat # UFC710008 Fisher).

The Centricon is pre washed with 30 mls of PBS at 1000×g for 3 minutes at room temperature. Next, 15-70 mls of the pre-spun cell culture supernatant is poured into the Concentrate Cup and is centrifuged in a Swing Bucket Adapter (Fisher Cat #75-008-144) for 30 minutes at 1000×g at room temperature.

The flow through in the Collection Cup is poured off. The volume in the Concentrate Cup is brought back up to 60 mls with any additional supernatant. The Concentrate Cup is centrifuged for 30 minutes at 1000×g at room temperature until all of the cell supernatant is concentrated.

The Concentrate Cup is washed by adding 70 mls of PBS and centrifuged for 30-60 minutes at 1000×g until approximately 2 mls remains. The exosomes are removed from the filter by inverting the concentrate into the small sample cup and centrifuge for 1 minute at 4° C. The volume is brought up to 25 mls with PBS. The exosomes are now concentrated and are added to a 30% Sucrose Cushion.

To make a cushion, 4 mls of Tris/30% Sucrose/D20 solution (30 g protease-free sucrose, 2.4 g Tris base, 50 ml D20, adjust pH to 7.4 with 10N NCL drops, adjust volume to 100 mls with D20, sterilize by passing thru a 0.22-um filter) is loaded to the bottom of a 30 ml V bottom thin walled Ultracentrifuge tube. The diluted 25 mls of concentrated exosomes is gently added above the sucrose cushion without disturbing the interface and is centrifuged for 75 minutes at 100,000×g at 4° C. The ˜25 mls above the sucrose cushion is carefully removed with a 10 ml pipet and the ˜3.5 mls of exosome is collected with a fine tip transfer pipet (SAMCO 233) and transferred to a fresh ultracentrifuge tube, where 30 mls PBS is added. The tube is centrifuged for 70 minutes at 100,000×g at 4° C. The supernatant is poured off carefully. The pellet is resuspended in 200 ul PBS and can be stored at 4° C. or used for assays. A BCA assay (1:2) can be used to determine protein content and Western blotting or electron micrography can be used to determine exosome purification.

### Example 2

### Purification of Exosomes from VCaP and 22Rv1

Exosomes from Vertebral-Cancer of the Prostate (VCaP) and 22Rv1, a human prostate carcinoma cell line, derived from a human prostatic carcinoma xenograft (CWR22R) were collected by ultracentrifugation by first diluting plasma with an equal volume of PBS (1 ml). The diluted fluid was transferred to a 15 ml falcon tube and centrifuged 30 minutes at 2000×g 4° C. The supernatant (˜2 mls) was transferred to an ultracentrifuge tube 5.0 ml PA thinwall tube (Sorvall #03127) and centrifuged at 12,000×g, 4° C. for 45 minutes.

The supernatant (˜2 mis) was transferred to a new 5.0 ml ultracentrifuge tubes and filled to maximum volume with addition of 2.5 mis PBS and centrifuged for 90 minutes at 110,000×g, 4° C. The supernatant was poured off without disturbing the pellet and the pellet resuspended with 1 ml PBS. The tube was filled to maximum volume with addition of 4.5 ml of PBS and centrifuged at 110,000×g, 4° C. for 70 minutes.

The supernatant was poured off without disturbing the pellet and an additional 1 ml of PBS was added to wash the pellet. The volume was increased to maximum volume with the addition of 4.5 mis of PBS and centrifuged at 110,000×g for 70 minutes at 4° C. The supernatant was removed with P-1000 pipette until 100 μl of PBS was in the bottom of the tube. The ˜90 μl remaining was removed with P-200 pipette and the pellet collected with the ˜10 μl of PBS remaining by gently pipetting using a P-20 pipette into the microcentrifuge tube. The residual pellet was washed from the bottom of a dry tube with an additional 5 μl of fresh PBS and collected into microcentrifuge tube and suspended in phosphate buffered saline (PBS) to a concentration of 500 μg/ml.

### Example 3

### Plasma Collection and Exosome Purification

Blood is collected via standard veinpuncture in a 7 ml K2-EDTA tube. The sample is spun at 400 g for 10 minutes in a 4° C. centrifuge to separate plasma from blood cells (SORVALL Legend RT+centrifuge). The supernatant (plasma) is transferred by careful pipetting to 15 ml Falcon centrifuge tubes. The plasma is spun at 2,000 g for 20 minutes and the supernatant is collected.

For storage, approximately 1 ml of the plasma (supernatant) is aliquoted to a cryovials, placed in dry ice to freeze them and stored in −80° C. Before exosome purification, if samples were stored at −80° C., samples are thawed in a cold water bath for 5 minutes. The samples are mixed end over end by hand to dissipate insoluble material.

In a first prespin, the plasma is diluted with an equal volume of PBS (example, approximately 2 ml of plasma is diluted with 2 ml of PBS). The diluted fluid is transferred to a 15 ml Falcon tube and centrifuged for 30 minutes at 2000×g at 4° C.

For a second prespin, the supernatant (approximately 4 mis) is carefully transferred to a 50 ml Falcon tube and centrifuged at 12,000×g at 4° C. for 45 minutes in a Sorval.

In the isolation step, the supernatant (approximately 2 mis) is carefully transferred to a 5.0 ml ultracentrifuge PA thinwall tube (Sorvall #03127) using a P1000 pipette and filled to maximum volume with an additional 0.5 mis of PBS. The tube is centrifuged for 90 minutes at 110,000×g at 4° C.

In the first wash, the supernatant is poured off without disturbing the pellet. The pellet is resuspended or washed with 1 ml PBS and the tube is filled to maximum volume with an additional 4.5 ml of PBS. The tube is centrifuged at 110,000×g at 4° C. for 70 minutes. A second wash is performed by repeating the same steps.

The exosomes are collected by removing the supernatant with P-1000 pipette until approximately 100 μl of PBS is in the bottom of the tube. Approximately 90 μl 1 of the PBS is removed and discarded with P-200 pipette. The pellet and remaining PBS is collected by gentle pipetting using a P-20 pipette. The residual pellet is washed from the bottom of the dry tube with an additional 5 μl of fresh PBS and collected into a microcentrifuge tube.

### Example 4

### Analysis of Exosomes Using Antibody-Coupled Microspheres and Directly Conjugated Antibodies

This example demonstrates the use of particles coupled to an antibody, where the antibody captures the exosomes (see for example, FIG. 64B). An antibody, the detector antibody, is directly coupled to a label, and is used to detect a biomarker on the captured exosome.

First, an antibody-coupled microsphere set is selected (Luminex, Austin, Tex.). The microsphere set can comprise various antibodies, and thus allows multiplexing. The microspheres are resuspended by vortex and sonication for approximately 20 seconds. A Working Microsphere Mixture is prepared by diluting the coupled microsphere stocks to a final concentration of 100 microspheres of each set/μL in Startblock (Pierce (37538)). (Note: 50 μL, of Working Microsphere Mixture is required for each well.) Either PBS-1% BSA or PBS-BN (PBS, 1% BSA, 0.05% Azide, pH 7.4) may be used as Assay Buffer.

A 1.2 μm Millipore filter plate is pre-wet with 100 μl/well of PBS-1% BSA (Sigma (P3688-10PAK+0.05% NaAzide (S8032))) and aspirated by vacuum manifold. An aliquot of 50 μl of the Working Microsphere Mixture is dispensed into the appropriate wells of the filter plate (Millipore Multiscreen HTS (MSBVN1250)). A 50 μl aliquot of standard or sample is dispensed into to the appropriate wells. The filter plate is covered and incubated for 60 minutes at room temperature on a plate shaker. The plate is covered with a sealer, placed on the orbital shaker and set to 900 for 15-30 seconds to re-suspend the beads. Following that the speed is set to 550 for the duration of the incubation.

The supernatant is aspirated by vacuum manifold (less than 5 inches Hg in all aspiration steps). Each well is washed twice with 100 μl of PBS-1% BSA (Sigma (P3688-10PAK+0.05% NaAzide (S8032))) and is aspirated by vacuum manifold. The microspheres are resuspended in 50 μL of PBS-1% BSA (Sigma (P3688-10PAK+0.05% NaAzide (S8032))). The PE conjugated detection antibody is diluted to 4 μg/mL (or appropriate concentration) in PBS-1% BSA (Sigma (P3688-10PAK+0.05% NaAzide (S8032))). (Note: 50 μL of diluted detection antibody is required for each reaction.) A 50 μl aliquot of the diluted detection antibody is added to each well. The filter plate is covered and incubated for 60 minutes at room temperature on a plate shaker. The filter plate is covered with a sealer, placed on the orbital shaker and set to 900 for 15-30 seconds to re-suspend the beads. Following that the speed is set to 550 for the duration of the incubation. The supernatant is aspirated by vacuum manifold. The wells are washed twice with 100 μl of PBS-1% BSA (Sigma (P3688-10PAK+0.05% NaAzide (S8032))) and aspirated by vacuum manifold. The microspheres are resuspended in 100 μl of PBS-1% BSA (Sigma (P3688-10PAK+0.05% NaAzide (S8032))). The microspheres are analyzed on a Luminex analyzer according to the system manual.

### Example 5

### Analysis of Exosomes Using Antibody-Coupled Microspheres and Biotinylated Antibody

This example demonstrates the use of particles coupled to an antibody, where the antibody captures the exosomes. An antibody, the detector antibody, is biotinylated. A label coupled to streptavidin is used to detect the biomarker.

First, the appropriate antibody-coupled microsphere set is selected (Luminex, Austin, Tex.). The microspheres are resuspended by vortex and sonication for approximately 20 seconds. A Working Microsphere Mixture is prepared by diluting the coupled microsphere stocks to a final concentration of 50 microspheres of each set/μL in Startblock (Pierce (37538)). (Note: 50 μl of Working Microsphere Mixture is required for each well.) Beads in Start Block should be blocked for 30 minutes and no more than 1 hour.

A 1.2 μm Millipore filter plate is pre-wet with 100 μl/well of PBS-1% BSA+Azide (PBS-BN)((Sigma (P3688-10PAK+0.05% NaAzide (S8032))) and is aspirated by vacuum manifold. A 50 μl aliquot of the Working Microsphere Mixture is dispensed into the appropriate wells of the filter plate (Millipore Multiscreen HTS (MSBVN1250)). A 50 μl aliquot of standard or sample is dispensed to the appropriate wells. The filter plate is covered with a seal and is incubated for 60 minutes at room temperature on a plate shaker. The covered filter plate is placed on the orbital shaker and set to 900 for 15-30 seconds to re-suspend the beads. Following that, the speed is set to 550 for the duration of the incubation.

The supernatant is aspirated by a vacuum manifold (less than 5 inches Hg in all aspiration steps). Aspiration can be done with the Pall vacuum manifold. The valve is place in the full off position when the plate is placed on the manifold. To aspirate slowly, the valve is opened to draw the fluid from the wells, which takes approximately 3 seconds for the 100 μl of sample and beads to be fully aspirated from the well. Once the entire sample is drained, the purge button on the manifold is pressed to release residual vacuum pressure from the plate.

Each well is washed twice with 100 μl of PBS-1% BSA+Azide (PBS-BN)(Sigma (P3688-10PAK+0.05% NaAzide (S8032))) and is aspirates by vacuum manifold. The microspheres are resuspended in 50 μl of PBS-1% BSA+Azide (PBS-BN)((Sigma (P3688-10PAK+0.05% NaAzide (S8032)))

The biotinylated detection antibody is diluted to 4 μg/mL in PBS-1% BSA+Azide (PBS-BN)((Sigma (P3688-10PAK+0.05% NaAzide (S8032))). (Note: 50 μl of diluted detection antibody is required for each reaction.) A 50 μl aliquot of the diluted detection antibody is added to each well.

The filter plate is covered with a sealer and is incubated for 60 minutes at room temperature on a plate shaker. The plate is placed on the orbital shaker and set to 900 for 15-30 seconds to re-suspend the beads. Following that, the speed is set to 550 for the duration of the incubation.

The supernatant is aspirated by vacuum manifold. Aspiration can be done with the Pall vacuum manifold. The valve is place in the full off position when the plate is placed on the manifold. To aspirate slowly, the valve is opened to draw the fluid from the wells, which takes approximately 3 seconds for the 100 ul of sample and beads to be fully aspirated from the well. Once all of the sample is drained, the purge button on the manifold is pressed to release residual vacuum pressure from the plate.

Each well is washed twice with 100 μl of PBS-1% BSA+Azide (PBS-BN)((Sigma (P3688-10PAK+0.05% NaAzide (S8032))) and is aspirated by vacuum manifold. The microspheres are resuspended in 50 μl of PBS-1% BSA (Sigma (P3688-10PAK+0.05% NaAzide (S8032))).

The streptavidin-R-phycoerythrin reporter (Molecular Probes 1 mg/ml) is diluted to 4 μg/mL in PBS-1% BSA+Azide (PBS-BN)(. (Note: 50 μl of diluted streptavidin-R-phycoerythrin is required for each reaction.) A 50 μl aliquot of the diluted streptavidin-R-phycoerythrin is added to each well.

The filter plate is covered with a sealer and is incubated for 60 minutes at room temperature on a plate shaker. The plate is placed on the orbital shaker and set to 900 for 15-30 seconds to re-suspend the beads. Following that, the speed is set to 550 for the duration of the incubation.

The supernatant is aspirated by vacuum manifold. Aspiration can be done with the Pall vacuum manifold. The valve is place in the full off position when the plate is placed on the manifold. To aspirate slowly, the valve is opened to draw the fluid from the wells, which takes approximately 3 seconds for the 100 ul of sample and beads to be fully aspirated from the well. Once all of the sample is drained, the purge button on the manifold is pressed to release residual vacuum pressure from the plate.

Each well is washed twice with 100 μl of PBS-1% BSA+Azide (PBS-BN)((Sigma (P3688-10PAK+0.05% NaAzide (S8032))) and is aspirated by vacuum manifold. The microspheres are resuspended in 100 μl of PBS-1% BSA+Azide (PBS-BN)((Sigma (P3688-10PAK+0.05% NaAzide (S8032))) and analyzed on the Luminex analyzer according to the system manual.

### Example 6

### Determining Bio-Signatures for Prostate Cancer Using Multiplexing

The exosomes samples obtained using methods as described in Example 1-3 are used in multiplexing assays as described in Examples 4 and 5. The detection antibodies used are CD63, CD9, CD81, B7H3 and EpCam. The capture antibodies used are CD9, PSCA, TNFR, CD63 2×, B7H3, MFG-E8, EpCam 2×, CD63, Rab, CD81, SETAP, PCSA, PSMA, 5T4, Rab IgG (control) and IgG (control), resulting in 100 combinations to be screened (FIG. 64C).

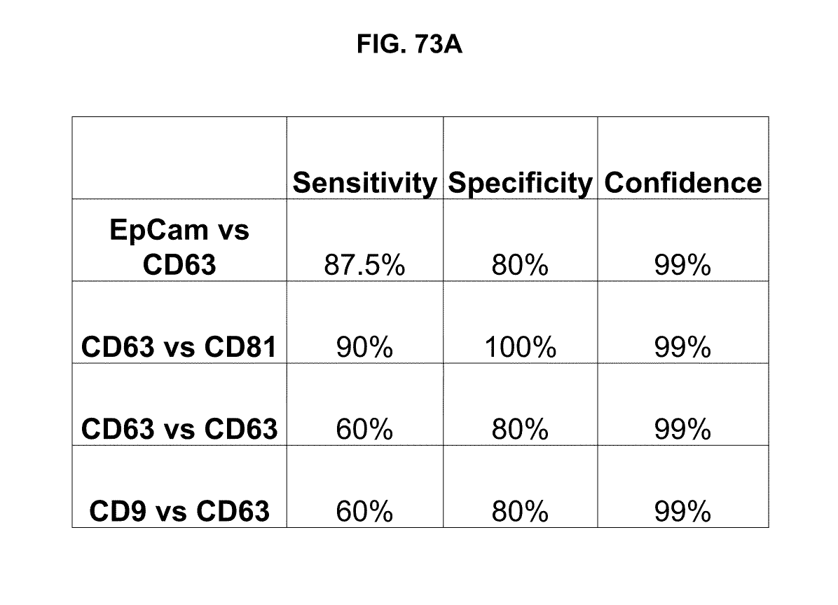

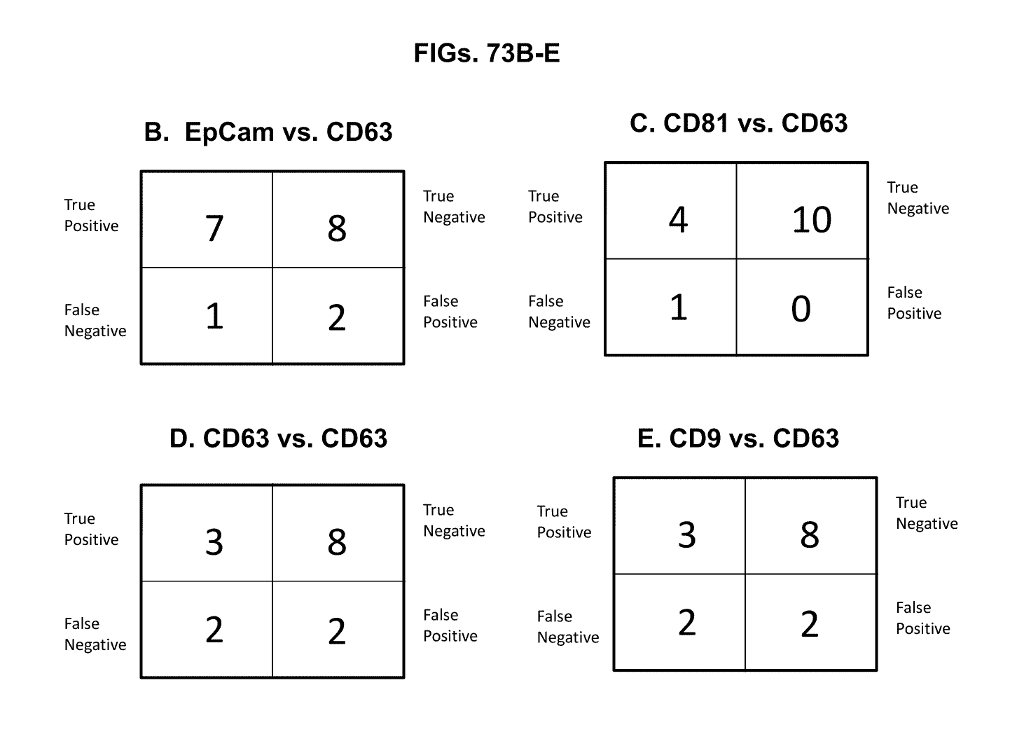

Ten prostate cancer patients and 12 normal control patients were screened. The results are depicted in FIGS. 68A-R and FIGS. 70A-D. FIGS. 70E-F depict the results of using PCSA capture antibodies (FIG. 70E) or EpCam capture antibodies (FIG. 70F), and detection using one or more detector antibodies. The sensitivity and specificity of the different combinations is depicted in FIGS. 73A-E.

### Example 7

### Determining Bio-Signatures for Colon Cancer Using Multiplexing

The exosomes samples obtained using methods as described in Example 3 is used in multiplexing assays as described in Examples 4 and 5. The detection antibodies used are CD63, CD9, CD81, B7H3 and EpCam. The capture antibodies used are CD9, PSCA, TNFR, CD63 2×, B7H3, MFG-E8, EpCam 2×, CD63, Rab, CD81, STEAP, PCSA, PSMA, 5T4, Rab IgG (control) and IgG (control), resulting in 100 combinations to be screened.

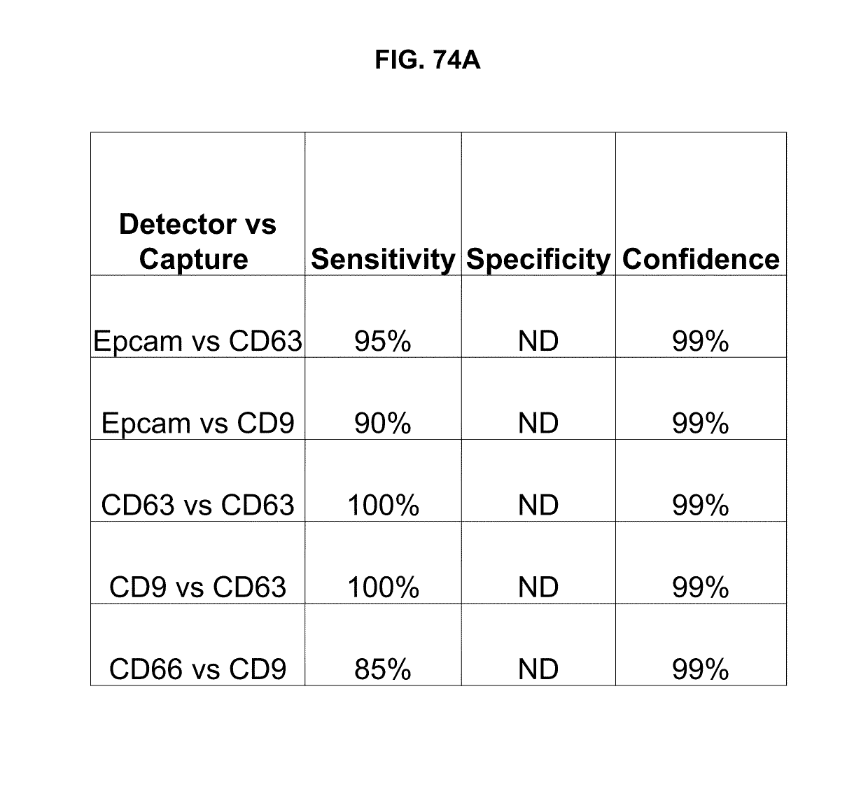

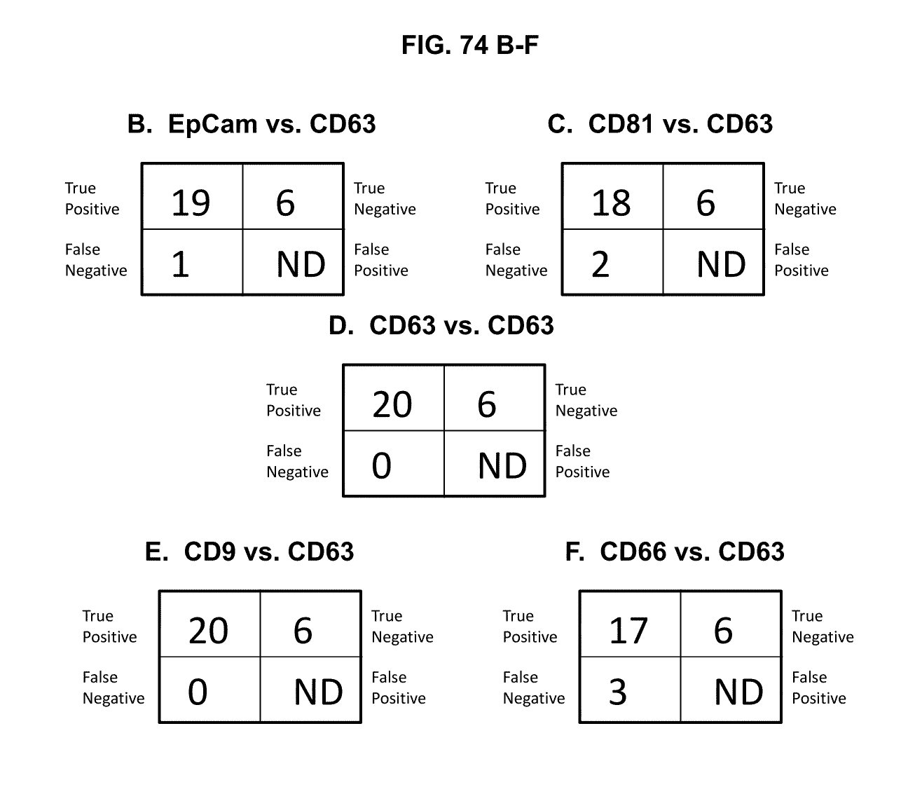

The results are depicted in FIGS. 69A-J, 71, and 72. The sensitivity of the different combinations is depicted in FIGS. 74A-F.

### Example 8

### Capture of Exosomes Using Magnetic Beads

Exosomes isolated as described in Example 2 are used. Approximately 40 ul of the exosomes are incubated with approximately 5 ug (˜50 μl) of EpCam antibody coated Dynal beads (Invitrogen, Carlsbad, Calif.) and 50 μl of Starting Block. The exosomes and beads are incubated with shaking for 2 hours at 45° C. in a shaking incubator. The tube containing the Dynal beads is placed on the magnetic separator for 1 minute and the supernatant removed. The beads are washed twice and the supernatant removed each time. Wash beads twice, discarding the supernatant each time.

### Example 9

### Detection of TMPRSS2:ERG in Exosomes

The RNA from the bead-bound exosomes of Example 9 was isolated using the Qiagen miRneasy™ kit, (Cat. No. 217061), according to the manufacturer's instructions.

The exosomes are homogenized in QIAzol™ Lysis Reagent (Cat. No. 79306). After addition of chloroform, the homogenate is separated into aqueous and organic phases by centrifugation. RNA partitions to the upper, aqueous phase, while DNA partitions to the interphase and proteins to the lower, organic phase or the interphase. The upper, aqueous phase is extracted, and ethanol is added to provide appropriate binding conditions for all RNA molecules from 18 nucleotides (nt) upwards. The sample is then applied to the RNeasy™ Mini spin column, where the total RNA binds to the membrane and phenol and other contaminants are efficiently washed away. High quality RNA is then eluted in RNase-free water.

RNA from the VCAP bead captured exosomes was measured with the Taqman TMPRSS:ERG fusion transcript assay (Kirsten D. Mertz et al. Neoplasia. 2007 March; 9(3): 200-206.). RNA from the 22Rv1 bead captured exosomes was measured with the Taqman SPINK1 transcript assay (Scott A. Tomlins et al. Cancer Cell 2008 Jun. 13(6):519-528). The GAPDH transcript (control transcript) was also measured for both sets of exosomal RNA.

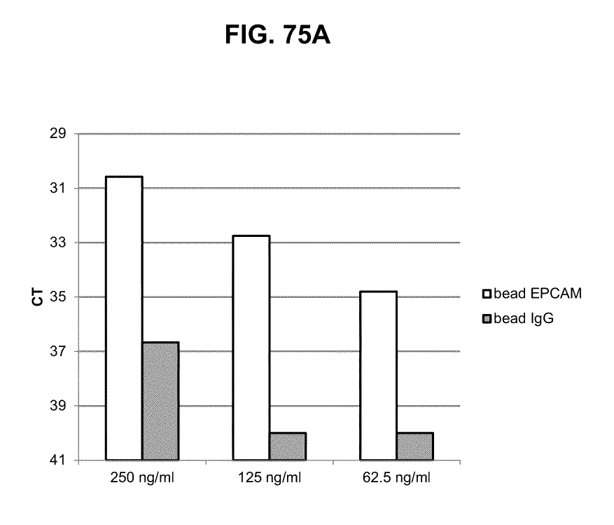

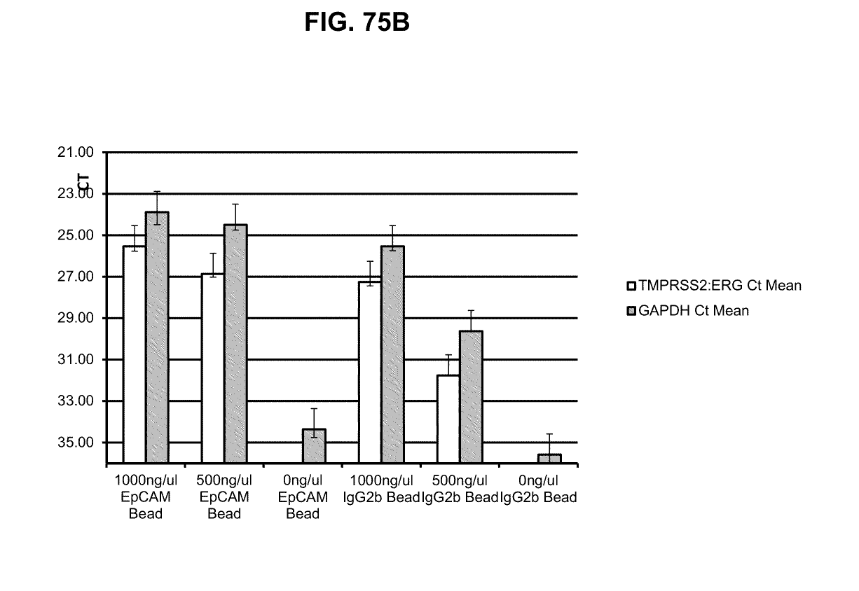

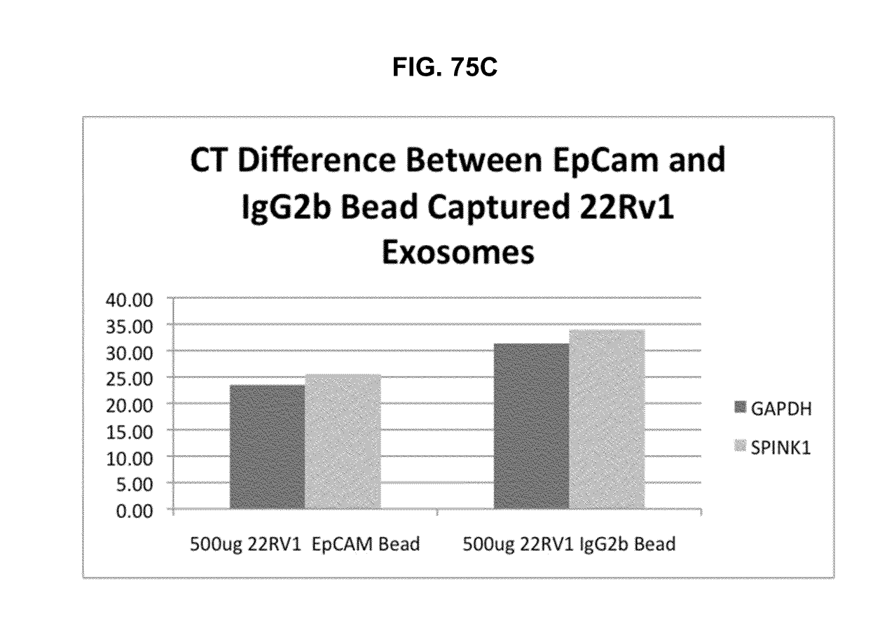

Higher CT values indicate lower transcript expression. One change in cycle threshold (CT) is equivalent to a 2 fold change, 3 CT difference to a 4 fold change, and so forth, which can be calculated with the following: 2̂^{CT1-CT2}. This experiment shows a difference in CT of the expression of the fusion transcript TMPRSS:ERG and the equivalent captured with the IgG2 negative control bead (FIGS. 75A-B). The same comparison of the SPINK1 transcript in 22RV1 exosomes shows a CT difference of 6.14 for a fold change of 70.5 (FIG. 75C).

### Example 10

### MicroRNA Profiles in Exosomes

Exosomes were collected by ultracentrifugation from 22Rv1, LNCaP, Vcap and normal plasma (pooled from 16 donors) as described in Examples 1 and 2. RNA was extracted using the Exiqon miR isolation kit (Cat. No. 300110, 300111). Equals amounts of exosomes (30 μg) were used as determined by BCA assay.

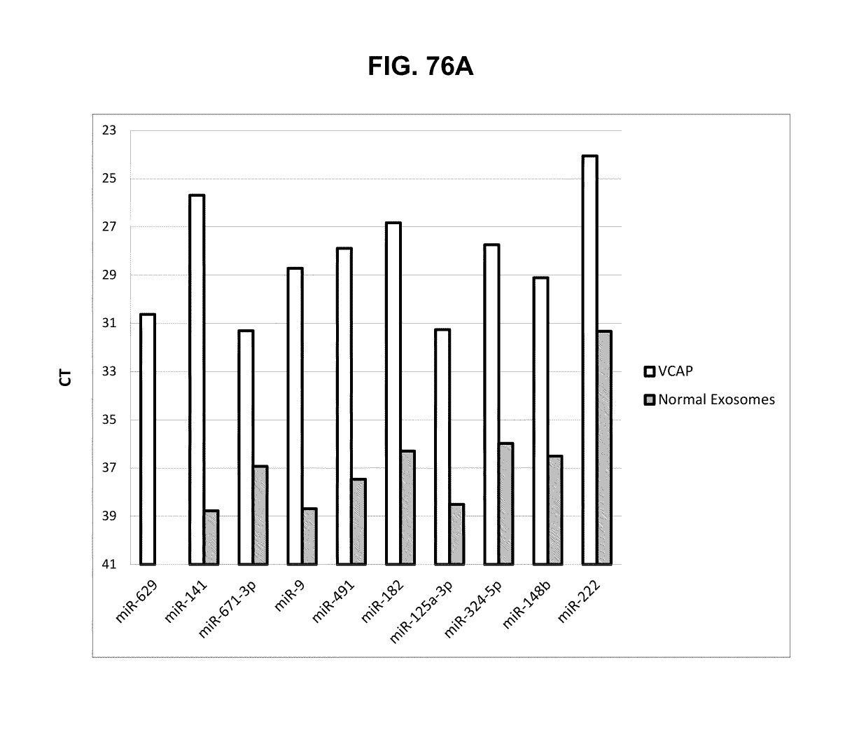

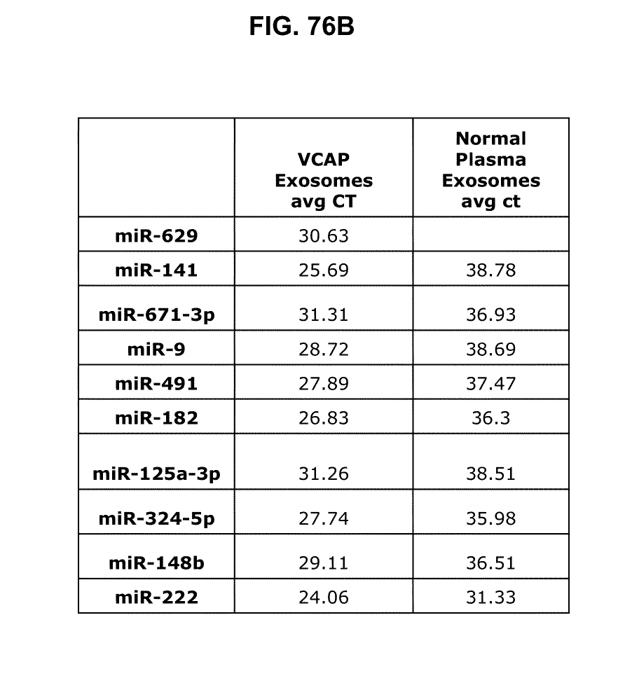

Equal volumes (5 μl) were put into a reverse-transcription reaction for microRNA. The reverse-transcriptase reactions were diluted in 81 μl of nuclease-free water and then 9 μl of this solution was added to each individual miR assay. MiR-629 was found to only be expressed in PCa (prostate cancer) exosomes and was virtually undetectable in normal plasma exosomes. MiR-9 was found to be highly overexpressed (˜704 fold increase over normal as measured by copy number) in all PCa cell lines, and has very low expression in normal plasma exosomes. The top ten differentially expressed miRNAs are depicted in FIGS. 76A-B.

### Example 11

### MicroRNA Profiles of Magentic EpCam-Captured Exosomes

The bead-bound exosomes of Example 9 was placed in QIAzol™ Lysis Reagent (Cat. #79306). An aliquot of 125 fmol of c. elegans miR-39 was added. The RNA from the exosomes was isolated using the Qiagen miRneasy™ kit, (Cat. #217061), according to the manufacturer's instructions, and eluted in 30 ul RNAse free water.

10 μl of the purified RNA was placed into a pre-amplification reaction for miR-9, miR-141 and miR-629 using a Veriti 96-well thermocycler. A 1:5 dilution of the pre-amplification solution was used to set up a qRT-PCR reaction for miR9 (ABI 4373285), miR-141 (ABI 4373137) and miR-629 (ABI 4380969) as well as c. elegans miR-39 (ABI 4373455). The results were normalized to the c. elegans results for each sample.

### Example 12

### MicroRNA Profiles of CD9-Captured Exosomes

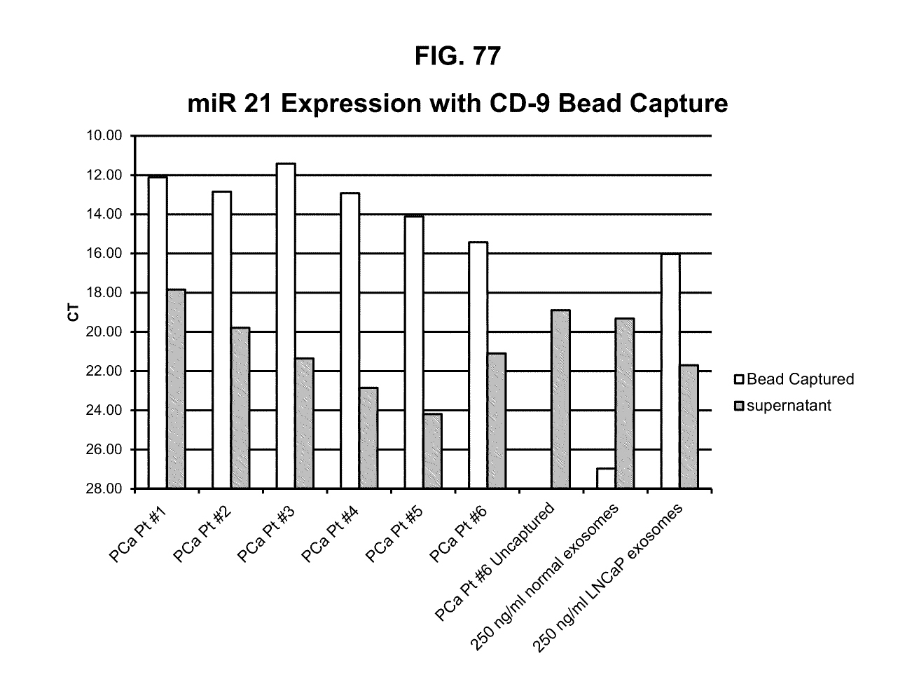

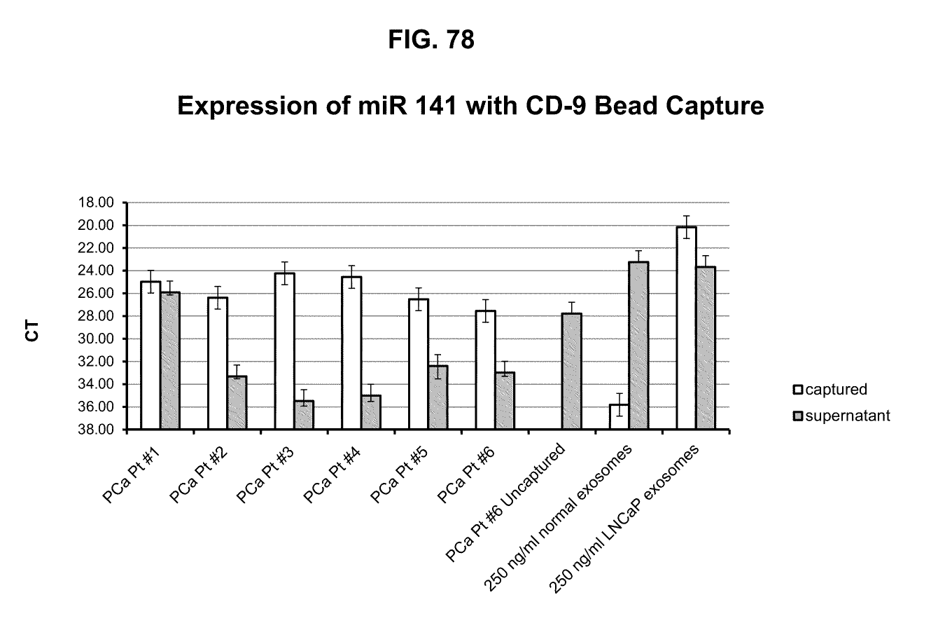

The CD9 coated Dynal beads (Invitrogen, Carlsbad, Calif.) were used instead of EpCam coated beads as in Example 12. Exosomes from prostate cancer patients, LNCaP, or normal purified exomes were incubated with the CD9 coated beads and the RNA isolated as described in Example 12. The expression of miR-21 and miR-141 was detected by qRT-PCR and the results depicted in FIGS. 77 and 78.

### Example 13

### Reference Values for Prostate Cancer

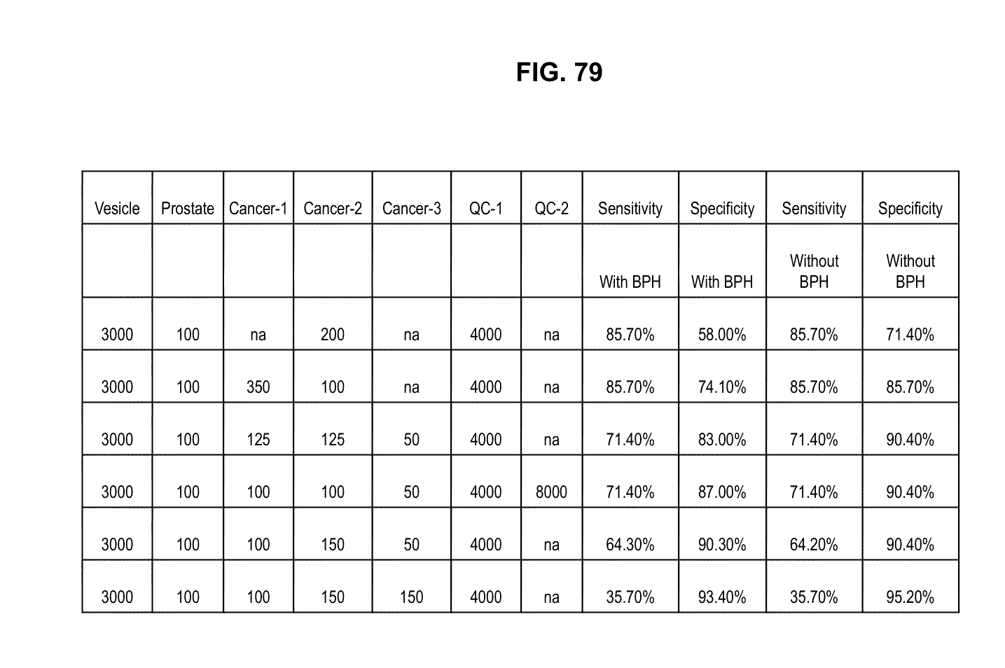

Fourteen stage 3 prostate cancer subjects, eleven benign prostate hyperplasia (BPH) samples, and 15 normal samples were tested. Exosome samples were obtained using methods as described in Example 3 and used in multiplexing assays, such as described in Examples 4 and 5. The samples were analyzed to determine four criteria 1) if the sample has overexpressed exosomes, 2) if the sample has overexpressed prostate exosomes, 3) if the sample has overexpressed cancer exosomes, and 4) if the sample is reliable. If the sample met all four criteria, the categorization of the sample as positive for prostate cancer had varying sensitivities and specificities, depending on the different bio-signatures present for a sample as described below (Cancer-1, Cancer-2, and Cancer-3, FIG. 79). The four criteria were as follows:

Exosome Overexpression

The mean fluorescence intensities (MFIs) for a sample in three assays were averaged to determine a value for the sample. Each assay used a different capture antibody. The first used a CD9 capture antibody, the second a CD81 capture antibody, and the third a CD63 antibody. The same combination of detection antibodies was used for each assay, antibodies for CD9, CD81, and CD63. If the average value obtained for the three assays was greater than 3000, the sample was categorized as having overexpressed exosomes (FIG. 79, Exosome).

Prostate Exosome Overexpression

The MFIs for a sample in two assays were averaged to determine a value for the sample. Each assay used a different capture antibody. The first used a PCSA capture antibody and the second used a PSMA capture antibody. The same combination of detection antibodies was used for each assay, antibodies for CD9, CD81, and CD63. If the average value obtained for the two assays was greater than 100, the sample was categorized as having prostate exosomes overexpressed (FIG. 79, Prostate).

Cancer Exosome Overexpression

Three different cancer bio-signatures were used to determine if cancer exosomes were overexpressed in a sample. The first, Cancer-1, used an EpCam capture antibody and detection antibodies for CD81, CD9, and CD63. The second, Cancer-2, used a CD9 capture antibody with detection antibodies for EpCam and B7H3. If the MFI value of a sample for any two of the three cancer bio-signatures was above a reference value, the sample was categorized as having overexpressed cancer (see FIG. 79, Cancer-1, Cancer-2, Cancer-3).

Reliability of Sample

Two quality control measures, QC-1 and QC-2, were determined for each sample. If the sample met one of them, the sample was categorized as reliable.

For QC-1, the sum of all the MFIs of 7 assays was determined Each of the 7 assays used detection antibodies for CD59 and PSMA. The capture antibody used for each assay was CD63, CD81, PCSA, PSMA, STEAP, B7H3, and EpCam. If the sum was greater than 4000, the sample was not reliable and not included.

For QC-2, the sum of all the MFIs of 5 assays was determined Each of the 5 assays used detection antibodies for CD9, CD81 and CD63. The capture antibody used for each assay was PCSA, PSMA, STEAP, B7H3, and EpCam. If the sum was greater than 8000, the sample was not reliable and not included.

The sensitivity and specificity for samples with BPH and without BPH samples after a sample met the criteria as described herein, are shown in FIG. 79.

### Example 14

### Obtaining Serum Samples from Subjects

Blood is collected from subjects (both healthy subjects and subjects with prostate cancer) in EDTA tubes, citrate tubes or in a 10-ml Vacutainer SST plus Blood Collection Tube (BD367985 or BD366643, BD Biosciences). Blood is processed for plasma isolation within 2 h of collection.

Samples are allowed to sit at room temperature for a minimum of 30 min and a max of 2 h. Separation of the clot is accomplished by centrifugation at 1,000-1,300×g at 4° C. for 15-20 min. The serum is removed and dispensed in aliquots of 500 μl into 500- to 750-μl cryo-tubes. Specimens are stored at −80° C.

At a given sitting, the amount of blood drawn can range from ˜20 to ˜90 ml. Blood from several EDTA tubes is pooled and transferred to RNase/DNase-free 50-ml conical tubes (Greiner), and centrifuged at 1,200×g at room temperature in a Hettich Rotanta 460R benchtop centrifuge for 10 min. Plasma is transferred to a fresh tube, leaving behind a fixed height of 0.5 cm plasma supernatant above the pellet to avoid disturbing the pellet. Plasma is aliquoted, with inversion to mix between each aliquot, and stored at −80° C.

### Example 15

### RNA Isolation From Human Plasma and Serum Samples

Four hundred μl of human plasma or serum is thawed on ice and lysed with an equal volume of 2× Denaturing Solution (Ambion). RNA is isolated using the mirVana PARIS kit following the manufacturer's protocol for liquid samples (Ambion), modified such that samples are extracted twice with an equal volume of acid-phenol chloroform (as supplied by the Ambion kit). RNA is eluted with 105 μl of Ambion elution solution according to the manufacturer's protocol. The average volume of eluate recovered from each column is about 80 μl.

A scaled-up version of the mirVana PARIS (Ambion) protocol is also used: 10 ml of plasma is thawed on ice, two 5-ml aliquots are transferred to 50-ml tubes, diluted with an equal volume of mirVana PARIS 2× Denaturing Solution, mixed thoroughly by vortexing for 30 s and incubated on ice for 5 min. An equal volume (10 ml) of acid/phenol/chloroform (Ambion) is then added to each aliquot. The resulting solutions are vortexed for 1 min and spun for 5 min at 8,000 rpm, 20° C. in a JA17 rotor. The acid/phenol/chloroform extraction is repeated three times. The resulting aqueous volume is mixed thoroughly with 1.25 volumes of 100% molecular-grade ethanol and passed through a mirVana PARIS column in sequential 700-μl aliquots. The column is washed following the manufacturer's protocol, and RNA is eluted in 105 μl of elution buffer (95° C.). A total of 1.5 μl of the eluate is quantified by Nanodrop.

### Example 16

### Measurement of miRNA Levels in RNA from Plasma and Serum by Using TaqMan qRT-PCR Assays

A fixed volume of 1.67 μl of RNA solution from about ˜80 μl-eluate from RNA isolation of a given sample is used as input into the reverse transcription (RT) reaction. For samples in which RNA is isolated from a 400-μl plasma or serum sample, for example, 1.67 μl of RNA solution represents the RNA corresponding to (1.67/80)×400=8.3 μl plasma or serum. For generation of standard curves of chemically synthesized RNA oligonucleotides corresponding to known miRNAs, varying dilutions of each oligonucleotide are made in water such that the final input into the RT reaction has a volume of 1.67 μl. Input RNA is reverse transcribed using the TaqMan miRNA Reverse Transcription Kit and miRNA-specific stem-loop primers (Applied BioSystems) in a small-scale RT reaction comprised of 1.387 μl of H2O, 0.5 μl of 10× Reverse-Transcription Buffer, 0.063 μl of RNase-Inhibitor (20 units/μl), 0.05 μl of 100 mM dNTPs with dTTP, 0.33 μl of Multiscribe Reverse-Transcriptase, and 1.67 μl of input RNA; components other than the input RNA can be prepared as a larger volume master mix, using a Tetrad2 Peltier Thermal Cycler (BioRad) at 16° C. for 30 min, 42° C. for 30 min and 85° C. for 5 min. Real-time PCR is carried out on an Applied BioSystems 7900HT thermocycler at 95° C. for 10 min, followed by 40 cycles of 95° C. for 15 s and 60° C. for 1 min. Data is analyzed with SDS Relative Quantification Software version 2.2.2 (Applied BioSystems), with the automatic Ct setting for assigning baseline and threshold for Ct determination.

The protocol can also be modified to include a preamplification step, such as for detecting miRNA. A 1.25-μl aliquot of undiluted RT product is combined with 3.75 μl of Preamplification PCR reagents [comprised, per reaction, of 2.5 μl of TaqMan PreAmp Master Mix (2×) and 1.25 μl of 0.2× TaqMan miRNA Assay (diluted in TE)] to generate a 5.0-μl preamplification PCR, which is carried out on a Tetrad2 Peltier Thermal Cycler (BioRad) by heating to 95° C. for 10 min, followed by 14 cycles of 95° C. for 15 s and 60° C. for 4 min. The preamplification PCR product is diluted (by adding 20 μl of H₂O to the 5-μl preamplification reaction product), following which 2.25 μl of the diluted material is introduced into the real-time PCR and carried forward as described.

### Example 17

### Generation of Standard Curves for Absolute Quantification of miRNAs

Synthetic single-stranded RNA oligonucleotides corresponding to the mature miRNA sequence (miRBase Release v.10.1) are purchased from Sigma. Synthetic miRNAs are input into the RT reaction over an empirically-derived range of copies to generate standard curves for each of the miRNA TaqMan assays listed above. In general, the lower limit of accurate quantification for each assay is designated based on the minimal number of copies input into an RT reaction that results in a Ct value within the linear range of the standard curve and that is also not equivalent to or higher than a Ct obtained from an RT input of lower copy number. A line is fit to data from each dilution series using Ct values within the linear range, from which y=mln(x)+b equations are derived for quantification of absolute miRNA copies (x) from each sample Ct (y). Absolute copies of miRNA input into the RT reaction are converted to copies of miRNA per microliter plasma (or serum) based on the knowledge that the material input into the RT reaction corresponds to RNA from 2.1% of the total starting volume of plasma [i.e., 1.67 μl of the total RNA eluate volume (80 μl on average) is input into the RT reaction]. An example of a synthetic miRNA sequence is for miR-141, 5′UAACACUGUCUGGUAAAGAUGG3′ (SEQ ID NO. 1), which can be obtained commercially such as from Sigma (St. Louis, Mo.).

### Example 18

### Identification of Gene Expression Profiles for Prostate Cancer Using Immunohistochemistry Analysis

Samples of solid tumor are excised and subjected to fixation and embedded in paraffin. The tumor block is cut into sections for placement on a glass slide. The slide is stained with the designated primary antibody which reacts with the tissue antigen as chosen by the pathologist. A labeled secondary antibody is reacted with the primary antibody and coupled to a streptavidin-horseradish peroxidase. This complex is reacted with a chromogen to produce a colored stain. The stained slides are viewed by a pathologist under a light microscope. The pathologist performs a semi-quantitative interpretation of the intensity of the staining Typically, a 0 to 4 scale is utilized with 0 representing no staining or negative result. The pathologist then estimates the proportion of the tumor cells that are stained positively. Typically, a 0 to 100% scale is utilized. Each antibody interpretation is annotated by the pathologist into the patient report. Results of the analysis of the 22 prostate cancer samples shows the genes overexpressed in at least 10 of the 22 samples are androgen receptor, EGFR, HSP90, and SPARC (Table 3).

TABLE 3
Genes Mostly Commonly Overexpressed in Prostate Cancer

| Genes from Expression Profiling Array | Frequency |
|---|---|
| for 14 Prostate Cancer Samples | of Occurrence |
| DNMT3B | 8/14 |
| Androgen Receptor | 6/14 |
| GART | 7/14 |
| MGMT | 6/14 |
| SSTR3 | 6/14 |
| TOP2B | 9/14 |

### Example 19

### Tissue Preparation for Identification of Gene Expression Profiles for Prostate Cancer

Tissue Preparation

Before starting, and using powder-free gloves, the work area is thoroughly cleaned with either RNaseAway® (Sigma Cat. No. 83931) or 70% ethanol (70% 200 proof ethanol and 30% pure water). Frozen tissue from the −80° C. freezer is removed and is immediately transferred to a tray containing dry ice, the tissue does not remain at room temperature for any length of time. Particularly if the tissue is small, as thawing could occur quickly and consequently the RNA would degrade irreversibly.

A sterile 100 mm diameter Petri dish (plastic) or tissue culture dish is placed on the clean ice to pre-chill, as well as the clean serrated tip forceps, and a new, clean heavy duty razor blade.

The tissue in the dish, if wrapped in foil or other material, is carefully unwrapped while in contact with the ice to prevent it from thawing. Even partial thawing of the tissue (which could happen is seconds) will irreversibly degrade the RNA, compromising the quality of the microarray assay or making it difficult to assay.

While using pre-chilled forceps and a razor blade, small pieces are cut off of the tissue so the sections to be used for microarray are not larger than approximately 1 mm thick. Often “shaving” off parts of the tumor is the easiest and fastest method. The forceps and razor blade are chilled every few seconds on a piece of dry ice so they remain very cold when in contact with the tissue. About 100-400 mg of tissue, roughly 20 mm³, no larger than “pea-size” is used.

The tissue cuttings are carefully placed into an anti-static weight dish (preferably the “pour boat type”) previously chilled on the dry ice. Then, the tissue is quickly transferred from the weigh dish to a pre-chilled borosilicate tube that has been previously marked with the appropriate specimen number, ensuring the cut tissue pieces do not stick to the walls of the tube, since they would rapidly thaw. Keeping the tube very cold and upright when transferring tissue to it is the best way to avoid that.

Any leftover tissue should be kept frozen on the dry ice until returned to a −80° C. freezer.

Homogenization Using the Covaris Tissue Processor

First, the circulating water bath (Multitemp III) is turned on, so it starts cooling off the water. Make sure the water bath contains enough water (ultrapure water only). The Covaris S-2 instrument is turned on. The water chamber of Covaris system is filled with 95% ultrapure water and 5% tap water. The computer connected to Covaris S-2 instrument is turned on, and the SonoLab software is opened.

The degassing process is turned on by clicking on the “degas” button within the SonoLab window; water should be degassed and pre-chilled (by the Multitemp III chiller water bath) for about 30 minutes, so temperature will remain between 17 and 20° C. during the homogenization of the samples. Also, the degassing process should be running during the entire session, and turned off only when ready to shut down the SonoLab software and the Covaris S-2 instrument.

The previously cut frozen tissue remains in the Covaris borosilicate tube, on the dry ice, until everything is ready for homogenization.

The program “MPI/IGC Processing” in the SonoLab program is opened. (NOTE: The following steps are done very quickly so the frozen tissue remains frozen until the last second before homogenization. The longer the tissue is thawed in between steps, the more RNA degradation typically occurs.)

Using a filtered 1000 μL pipet tip and a P-1000 pipetor, 500 μL of RLT buffer (from the Qiagen RNeasy mini kit) is added to the frozen tissue Immediately, the screw cap is put back on and very quickly the tube is inserted into the tube holder in the Covaris S-2 instrument. 2-Mercaptoethanol is added to RLT before use. Ten μL of 2-Mercaptoethanol is added per 1 mL RLT buffer. Then the Start button to commence the homogenization is pressed.

The tube is removed after the process is completed, and is placed on wet ice. The cap is opened and 500 μL of TRIzol is added. The tube is then recapped and is quickly mixed by moving the tube side to side.

If RNA extraction is performed shortly after homogenization, the tubes remain on wet ice, otherwise all specimens are frozen in dry ice or at −80° C. until ready for RNA extraction.

When the homogenization session ends, the degassing is shut off, the water is removed from the water chamber in the Covaris S-2 instrument, then degas for about 2 seconds in order to purge the remaining water from the lines. The SonoLab program is closed first, and then the Multitemp III chiller water bath, and Covaris S-2 instrument are shut off.

### Example 20

### RNA Extraction and Purification for Identification of Gene Expression Profiles for Prostate Cancer

TRIzol Extraction.

If the previously homogenized tissue has been stored in the −80° C. freezer, the tissue is thawed at room temperature or 65° C. Using clean powder-free gloves, the work area is cleaned again thoroughly with either RNaseAway® (Sigma Cat. No. 83931) or 70% ethanol. (NOTE: the following steps will be performed at room temperature and with room temperature reagents, unless otherwise indicated)

Tube contents are transferred to a 2 mL screw-cap, sterile and RNAse-free tube, ensuring that the lids are well tightened. The sample is heated up in a digital heat block at 65° C. for 5 minutes. If previously frozen, the sample is incubated at 65° C. for 7 minutes.

The sample is then removed from heat, and immediately, 200 μL of chloroform is added while the tubes are still hot. The caps are tightened well and are then mixed by shaking vigorously for 15-30 sec (do not vortex or DNA molecules will be sheared and may contaminate the RNA).

The tubes are then cooled on ice for 5 minutes, then the tubes are centrifuged at 10,000×g for 10 minutes at room temperature. Slowly, and using a filtered tip, approximately 0.7 mL of the upper aqueous phase which contains the total RNA is removed and is placed in a new, labeled 1.5 mL tube.

0.7 mL of room-temperature 70% ethanol is then added to the homogenized lysate, and is mixed well by pipetting.

Purification of the RNA-Containing Aqueous Phase with RNeasy Mini or Micro Kit.

(NOTE: When processing needle biopsy samples, micro columns aree used to bind the RNA and carrier RNA added to the lysate. The RNeasy Micro kit (Qiagen Cat. No. 74004) contains poly-AN RNA to be added as carrier RNA. Before using for the first time, dissolve the carrier RNA (310 μg) in 1 mL RNase free water. Store this stock solution at −20° C., and use to make fresh dilutions for each set of RNA preps.)

To make a working solution (4 ng/μL) for 10 preps, 5 μL of the dissolved RNA is added to 34 μL of Buffer RLT and is mixed by pipetting. 6 μL of this diluted solution is added to 54 μL of Buffer RLT. The final concentration is 4 ng/μL.

Up to 0.7 mL of the sample, including any precipitate that may have formed, is applied to an RNeasy mini or micro column placed in a 2 mL collection tube. The tube is closed gently, and is centrifuged for 30 seconds at 8000×g. The remaining 0.7 mL of the sample mixture is added to the same RNeasy mini or micro column and again is centrifuged at 8000×g for 30 seconds.

0.7 mL of buffer RW1 is then added to the RNeasy mini or micro column. The tube is closed gently, and is centrifuged for 30 seconds at 8000×g to wash the column. The flow through and 2 mL collection tube is discarded.

Without touching the bottom part of the column, the RNeasy mini or micro column is transferred into a new 2 mL collection tube. 0.5 mL buffer RPE is pipetted onto the RNeasy mini or micro column. The tube is closed gently, and is centrifuged for 30 seconds at 8000×g to wash the column. The flow through is then discarded.

Again, 0.5 mL buffer RPE is added to the RNeasy column. The tube is closed gently, and is centrifuged for 2 minutes at 8000×g to dry the RNeasy silica-gel membrane. The flow through and the collection tube are then discarded.

To elute, the RNeasy mini or micro column are transferred to a new 1.5 mL collection tube (this tube is labeled with the case number). RNase free H₂O (30-40 μL for a mini column or 7-14 μL for a micro column) is pipetted above the center of the RNeasy silica-gel membrane, without touching it. The tube is closed and after 2-4 minutes, the tube is centrifuged at 16,100×g for 1 minute.

The mini or micro column is then discarded and the RNA is placed on ice.

1 μL of each sample is aliquoted into a PCR tube for bio-analyzing. The RNA concentration determined by measuring the optical density or absorbance in a spectrophotometer as follows: TE pH 8.0 is used as the diluent buffer and as the blank. A 1:100 dilution: 1 μL RNA with 99 μL TE pH 8.0 is made and the absorbance for 260 and 280 nm is read with an Agilent spectrophotometer using a quartz cuvette. The setting in the spectrophotometer is at “Ratio,” and the ration obtained is the absorbance at 260 over 280, which ideally ranges from 1.8 to 2.2. In case absorbance at 260 nm is out of the linear range (below 0.1 or above 1), the dilution of the RNA in TE is repeated either by increasing the quantity of RNA or diluting it further, respectively. The RNA is then place in a designated freezer at −80° C. until ready to proceed with RNA labeling.

### Example 21

### RNA Amplification and Fluorescent Labeling for Identification of Gene Expression Profiles for Prostate Cancer

Following RNA purification from a tissue, the amplification and labeling of this RNA is a key step in gene expression profiling using microarray analysis. This technique allows the use of purified total RNA as a template for the synthesis of complementary DNA (cDNA) by reverse transcription the first step in RNA amplification. Fluorescent complementary RNA (cRNA) is synthesized by in vitro transcription, using cDNA as a template while incorporating a nucleotide (CTP) coupled to a fluorescent cyanine dye (cyanine-3 (pink) or cyanine-5 (blue)). The resulting fluorescent RNA is then compared side by side with another RNA, labeled with a different cyanine dye, by hybridizing both to a cDNA array.

A) cDNA Synthesis from Total RNA:

Before starting, and using powder-free gloves, the work area is cleaned thoroughly with either RNaseAway® (Sigma Cat. No. 83931) or 70% ethanol (70% 200 proof ethanol and 30% pure water). It is very important that the work area, the materials and equipment used are very clean, dust- and RNAse-free.

2 μg total RNA is added to a volume of 10.3 μL to a 0.2 mL microcentrifuge tube. The total concentration should be at least 5 ng/μL. When using more than 500 ng total RNA (or 10 ng or more or polyA+RNA) the total volume should be 6.5 μL.

3 μL of T7 Promoter Primer (from kit) is then added. Nuclease-free water is then used to bring the total reaction volume to 11.5 μL. The primer and the template are denatured by incubating the reaction at 65° C. in a thermal cycler for 10 minutes. The reactions are incubated at 4° C. for 5 minutes (this can be done on ice or in the thermal cycler).

Immediately-prior to use, the following components shown in Table 4 are gently mixed by pipetting, in the order indicated, at room temperature (pre-warm the 5× First Strand Buffer by incubating the vial in an 80° C. heat block for 1-2 minutes). To ensure optimal re-suspension, vortex briefly and spin the tube briefly in a microcentrifuge at full speed to drive the contents off the walls and lid. Keep at room temperature until use.

TABLE 4
cDNA Master Mix

| Component | Vol. (μL/rxn) | Vol. (μL/6.5 rxn) |
|---|---|---|
| 5X First Strand Buffer | 4.0 | 26 |
| 0.1M DTT | 2.0 | 13 |
| 10 mM dNTP mix | 1.0 | 6.5 |
| MMLV RT | 1.0 | 6.5 |
| RNaseOUT | 0.5 | 3.3 |
| TOTAL VOLUME | 8.5 | 55.3 |

To each sample tube, 8.5 μL of the cDNA Master Mix is added. The tubes are then vortexed at a low setting with short pulses in order to avoid bubble formation. The presence of bubbles could lead to enzyme denaturation thereby impairing enzyme activity.

The samples are then incubated at 40° C. in a thermal cycler for 2 hours. The temperature of the thermocycler is then switched to 65° C. and the samples are incubated for 15 minutes (incubation at 65° C. inactivates MMLV-RT (Moloney murine leukemia virus reverse transcriptase)).

The reactions are then incubated at 4° C. for 5 minutes (this can be done on ice or in the thermal cycler). The samples are spun briefly in a microcentrifuge at full speed to drive tube contents off the tube wall and lid.

B. Fluorescent cRNA Synthesis: In Vitro Transcription and Incorporation of Cyanine 3- or Cyanine 5-CTP

To each sample tube, either 2.4 μL cyanine 3-CTP (10 mM) or 2.4 μL cyanine 5-CTP (10 mM) is added. Cyanine 3 is bright pink and cyanine 5 is bright blue. Both are light sensitive and thus light exposure should be minimized. The cyanine 3-CTP (pink) is typically used for normal reference RNA labeling, and cyanine 5-CTP (blue) for the patient (tumor) RNA labeling. The 50% PEG (polyethylene glycol) solution is pre-warmed by incubating the vial in a 40° C. heat block for one minute. To ensure optimal re-suspension, vortex briefly and spin the tube briefly in a microcentrifuge at full speed to drive the contents off the tube walls and lid. The tube is kept at room temperature until use.

A Transcription Master Mix is made as shown in Table 5 Immediately-prior to use, quickly spin all tubes containing reaction components to bring down contents (for a few seconds), and combine the following components in the order indicated, at room temperature (then gently vortex Master Mix on a low setting, and spin in a microcentrifuge at full speed before adding to sample tubes). (Note: The enzymes are not added until just before performing the reaction).

TABLE 5
Transcription Master Mix

| Component | Vol. (μL/rxn) | Vol. (μL/6.5 rxn) |
|---|---|---|
| Nuclease-free water | 15.3 | 99.4 |
| 4X Transcription Buffer | 20 | 130 |
| 0.1M DTT | 6.0 | 39 |
| NTP Mix | 8.0 | 52 |
| 50% PEG | 6.4 | 41.6 |
| RNA seOUT | 0.5 | 3.3 |
| Inorganic Pyrophosphatase | 0.6 | 3.9 |
| T7 RNA Polymerase | 0.8 | 5.2 |
| TOTAL VOLUME | 57.6 | 374.4 |

To each sample tube, 57.6 μL of Transcription Master Mix is added and mixed by carefully vortexing at a low setting with short pulses in order to avoid bubble formation. The tubes are then quickly spun in a microcentrifuge at full speed to bring down contents of tube (for a few seconds).

The samples are then incubated in a thermal cycler bath at 40° C. for 2 hours.

C. Purification of Amplified cRNA

(Note: Remember to add four volumes of 100% ethanol to Buffer RPE before using the kit for the first time (See bottle label for specific volume)).

20 μL of nuclease free-water is added to the cRNA sample to obtain a total volume of 100 μL. 350 μL of Buffer RLT is added and is then mixed thoroughly by gently vortexing. 250 μL of ethanol (100% purity) is added and is then mixed thoroughly by vortexing. The sample is not centrifuged after.

700 μL of the cRNA sample is added to an RNeasy mini column in a 2 mL collection tube. The sample is centrifuged for 30 seconds at 13,000×g. After this first centrifugation, color should be present in the column membrane if the labeling is successful (pink for cyanine-3 and blue for cyanine-5).

The sample is passed through the column a second time. This allows the capture of labeled RNA not retained by the membrane in the first pass. The flow-through and collection tube is then discarded.

The RNeasy column is then transferred to a new collection tube and 500 μL of buffer RPE is added to the column. The sample is then centrifuged for 30 seconds at 13,000×g. The flow through is then discarded and the collection tube is re-used.

Again, 500 μL of Buffer RPE is added to the column. The sample is then centrifuged for 1 minute at 13,000×g, and the flow through and the collection tube is discarded.

The cleaned cRNA sample is eluted by transferring the RNeasy column to a new 1.5 mL collection tube. 30 μL of RNase-free water is added directly onto the RNeasy filter membrane. After 2-3 minutes the tube is centrifuged for 30 seconds at 13,000 rpm. The flow-through and the collection tube is retained (this is the labeled cRNA; a pink (cyanine 3) or blue (cyanine 5) color should be present).

The RNA concentration is determined by measuring the optical density or absorbance in a spectrophotometer (Agilent Technologies) as follows: TE pH 8.0 is used as the diluent buffer and as the blank. A 1:20 dilution: 4 μl RNA with 76 μl TE pH 8.0 is prepared. Absorbance for 260 (RNA), 550 (cyanine 3), and 650 (cyanine 5) nm in the Agilent spectrophotometer is determined using a quartz cuvette. The setting in the spectrophotometer is at “Spectrum/Peaks” and the range is from 220 to 700 nm. The absorbance corresponding to the RNA and the cyanine dye should then be used to calculate the quantity of RNA labeled and the efficiency of the cyanine dye incorporation.

### Example 22

### Hybridization with the Whole Human Genome Microarray for Identification of Gene Expression Profiles for Prostate Cancer

Hybridization of fluorescent complementary RNA (cRNA) to the 60-mer oligo microarray is a key step in gene expression profiling. By using Agilent microarray technology, the gene expression profile of a specimen of interest can be determined, and simultaneously compare two RNAs (i.e. tumor vs. normal) that have been previously labeled with different fluorescent dyes (cyanine 3 or cyanine 5).

Hybridization Procedure Using cRNA Labeled Targets

A) Preparation of 2×cRNA target solution to be used on a 4×44K Agilent oligo microarray

Before starting, and using powder-free gloves, the work area is cleaned thoroughly with either RNaseAway® (Sigma Cat. No. 83931) or 70% ethanol (70% 200 proof ethanol and 30% pure water). It is very important that the work area, the materials and equipment used are very clean, dust- and RNAse-free.

The 10× Blocking Agent (Agilent Cat. No. 5188-5281) is prepared (if using stock tube for the first time) by using an RNAse-free filtered pipette tip to add 0.5 mL of RNAse-free (or DEPC water) to the lyophilized pellet, mixing gently by vortexing, and centrifuging for 5-10 seconds. Once reconstituted with water, the 10× Blocking Agent should be stored frozen at −20° C. for up to 2 months.

To a 0.2 mL RNAse-free PCR tube nuclease-free water is added, bringing to 52.8 μL volume.

Using an RNAse-free filtered pipette tip, 825 ng of cyanine 3-labeled cRNA and 825 ng of cyanine 5-labeled cRNA (or more if the labeling efficiency of one of them was lower in order to add approximately equivalent quantities of cyanine dyes in both) is added.

Using an RNAse-free filtered pipette tip, 11 μL of 10× Blocking Agent is added.

This 2× Target solution may be quickly frozen in dry ice and stored in the dark in a −80° C. freezer up to 1 month.

B) cRNA Fragmentation and Preparation of 1× Hybridization Solution

To the 52.8 μL 2×cRNA Target solution, 2.2 μL of 25× Fragmentation buffer is added and is mixed gently by vortexing at a low speed before a quick centrifuge (5-10 seconds) to bring contents down from walls and tube lid.

The tube is incubated at 60° C. for 30 minutes in a thermal cycler such as the PTC-200 from MJ Research. This incubation fragments the cRNA to ideal size fragments that are optimal for hybridization. After the incubation, the tube is spun briefly in a microcentrifuge to drive the sample off the walls and lid.

55 μL of the 2×GE HI-RPM Hybridization Buffer is added and is then mixed well by careful pipetting, taking care to avoid introducing bubbles. The tube is then spun briefly in a microcentrifuge to drive the sample off the walls and lid before being used immediately.

The sample is placed on ice and is loaded onto the array as soon as possible.

C) Hybridization of Cyanine 3- and Cyanine 5-Labeled Samples to Agilent 4×44 K Oligo Microarrays

As many assembled stainless steel hybridization chambers, gasket slides and microarrays as necessary to complete the microarray hybridizations are procured.

Before loading each microarray with the hybridization mixture, the samples to be assayed are written down in a numerical order by writing down the barcode number of the corresponding microarray and the position (Array 1_{—}1, 1_{—}2, 1_{—}3, 1_{—}4) where each sample was loaded.

The first gasket is placed on the base of the first hybridization chamber base, making sure that the label of the gasket slide is facing up, and that it is well placed and flush with the chamber base. 100 μL of the hybridization solution is slowly drawn up from the first sample tube avoiding any bubbles, before “dispensing and dragging” it on the center of the gasket slide, so the solution will be slowly spread with the pipet tip throughout the gasket slide while dispensing it, but leaving approximately 2-3 mm space between the solution and the gasket that surrounds it.

Once the solution is dispensed, the hybridization chamber base with the gasket slide is not moved, and the microarray is placed over it as soon as possible.

The appropriate Agilent oligo microarray is removed from its packaging using clean, powder-free gloves. To avoid damaging the microarray surface, only the area where the barcode is placed and by the ends is where the microarray should be handled (a pair of Teflon-coated, slanted tip forceps can also be helpful when handling the microarrays and placing them over the gasket slide). It also helps removing the microarray from the plastic package while the numeric side is facing up (“Agilent side is down”), since it must be placed in this direction and it is easier to confirm that the right array (with the correct barcode number) is being assigned to that sample.

The array is carefully lowered and aligned with the 4 guide posts on the chamber base. Once aligned and slightly over (and parallel to) the gasket slide, the microarray slide is gently placed against the gasket slide to complete the sandwiched slide pair. The slides are quickly assessed to assure they are completely aligned and that the oligo microarray is not ajar.

The stainless steel chamber cover is placed onto the sandwiched slides, and then the clamp assembly is slid into place, until it comes to a stopping point in the middle of the chamber base and cover pair. The thumbscrew is tightened by turning it clockwise until it is fully handtight (without overtightening or using tools, as this may damage the parts and break the glass gasket slide and microarray.)

The chamber assembly is held vertically, and rotated slowly it clockwise 2-3 times in order to allow the hybridization solution to wet the gasket and the microarray. The sandwiched slides are inspected for bubble formation as a large mixing bubble should have formed. If stray, mixing bubbles are present and do not move as the chamber rotates, gently tap the chamber against your hand or other surface, and rotate chamber again (while in vertical position) to determine if the stationary bubbles are now moving. It is important that the stationary bubbles are dislodged before loading the assembled chamber into the hybridization rotator rack and oven.

Once all of the chambers are fully assembled, they are loaded into the hybridization rotator rack, ensuring the loaded hybridization chambers are in balance with others (can use an empty chamber as well) in the opposite position. The hybridization rotator rack is set to rotate at 10 rpm and the hybridization is at 65° C. for 17 hours.

D. Wash with Stabilization and Drying Solution.

Gene Expression Wash Buffer 2 is prewarmed to 37° C. as follows: 1000 mL of Gene Expression Wash Buffer 2 is dispensed directly into a sterile 1000-mL bottle, and is repeated until enough prewarmed Wash2 solution for the experiment is present. The 1000-mL bottle cap is tightened and placed in a 37° C. water bath the night before arrays.

Cyanine 5 is susceptible to degradation by ozone, thus, the following procedure is typically performed if the ozone levels in the laboratory exceed 5 ppb. (NOTE: Fresh Gene Expression Wash Buffer 1 and 2 should be used for each wash group (up to eight slides). The acetonitrile and Stabilization and Drying Solution may be reused for washing of up to three groups of slides.)

The Agilent Stabilization and Drying Solution contain an ozone scavenging compound dissolved in acetonitrile. The compound in solution is present in saturating amounts and may precipitate from the solution under normal storage conditions. If the solution shows visible precipitation, warming of the solution redissolves the compound. Washing slides using Stabilization and Drying Solution showing visible precipitation typically has a profound adverse effect on microarray performance.

The solution is slowly warmed in a water bath or a vented conventional oven at 40° C. in a closed container with sufficient head space to allow for expansion. If needed, the solution may be gently mixed to obtain a homogenous solution, under a vented fume hood away from open flames, or other sources of ignition. The solution is warmed only in a controlled and contained area that meets local fire code requirements.

After the precipitate is completely dissolved, the covered solution is left at room temperature, allowing it to equilibrate to room temperate prior to use. (NOTE: The original container can be used to warm the solution. The time needed to completely redissolve the precipitate is dependent on the amount of precipitate present, and may require overnight warming if precipitation is heavy. The Stabilization and Drying solution should not be filtered).

The Stabilization and Drying Solution should be set-up in a fume hood. Wash 1 and Wash 2 set-up areas should be placed close to, or preferably in, the same fume hood. Gloves and eye/face protection should be used in every step of the warming procedures.

The slide-staining dish #1 is completely filled with Gene Expression Wash Buffer 1 at room temperature. A slide rack is placed into slide-staining dish #2. A magnetic stir bar is then added and the slide-staining dish #2 is filled with enough Gene Expression Wash Buffer 1 at room temperature to cover the slide rack. This dish is placed on a magnetic stir plate.

The empty dish #3 is placed on the stir plate and a magnetic stir bar is added. The pre-warmed (37° C.) Gene Expression Wash Buffer 2 is not added until the first wash step has begun.

The slide-staining dish #4 is filled approximately three-fourths full with acetonitrile, a magnetic stir bar is added and this dish is placed on a magnetic stir plate.

The slide-staining dish #5 is filled approximately three-fourths full with Stabilization and Drying Solution, a magnetic stir bar added and this dish is placed on a magnetic stir plate.

The hybridization chamber is removed from incubator, and the hybridization chamber is prepared for disassembly. The hybridization chamber assembly is placed on a flat surface and the thumbscrew is loosened, turning counter-clockwise. The clamp assembly is slid off and the chamber cover removed.

With gloved fingers, the array-gasket sandwich is removed from the chamber base by grabbing the slides from their ends. Keeping the microarray slide numeric barcode facing up, the sandwich is quickly transferred to slide-staining dish #1.

Without letting go of the slides, the array-gasket sandwich is submerged into slide-staining dish #1 containing Gene Expression Wash Buffer 1. With the sandwich completely submerged in Gene Expression Wash Buffer 1, the sandwich is pried open from the barcode end only:

One of the blunt ends of the forceps is slipped between the slides, the forceps are turned gently upwards or downwards to separate the slides, letting the gasket slide drop to the bottom of the staining dish. The microarray slide is removed and placed into a slide rack in the slide-staining dish #2 containing Gene Expression Wash Buffer 1 at room temperature. Exposure of the slide to air should be minimized and only the barcode portion of the microarray slide or its edges should be touched.

When all slides in the group are placed into the slide rack in slide-staining dish #2, stirring is started using setting 4 for 1 minute. During this wash step, Gene Expression Wash Buffer 2 is removed from the 37° C. water bath and is poured into the Wash 2 dish. The slide rack is transferred to slide-staining dish #3 containing Gene Expression Wash Buffer 2 at elevated temperature and is stirred using setting 4 for 1 minute.

The slide rack from Gene Expression Wash Buffer 2 is removed and the rack is tilted slightly to minimize wash buffer carry-over. The slide rack is immediately transferred to the slide-staining dish #4 containing acetonitrile and is stirred using setting 4 for 30 seconds.

The slide rack is transferred to dish #5 filled with Stabilization and Drying Solution and is stirred using setting 4 for 1 minute.

The slide rack is slowly removed to minimize droplets on the slides. It should take 5 to 10 seconds to remove the slide rack. The used Gene Expression Wash Buffer 1 and Gene Expression Wash Buffer 2 are discarded.

The slides are scanned immediately to minimize the impact of environmental oxidants on signal intensities. If necessary, store slides in orange slide boxes in a N2 purge box, in the dark.

To scan the microarray slides, the scanner is turned on and after a few minutes the Agilent Scanner control is opened. The number of slides to be scanned (up to 48) is selected and after highlighting the rows that correspond to the slots to be scanned, Browse is selected and the output path or location where the image files will be saved is chosen.

To change any settings, click Settings>Modify Default Settings. A window pops up from which you can change the setting. The scanning resolution should be set up for 5 μm. The scanner reads the barcode and automatically names each file with that number.

When scanner status shows: “Scanner ready”, click Scan and each array takes approximately 7 minutes to be scanned. After all scans are finished, a report will automatically appear listing all serial numbers and the status of the scan (successful or not).

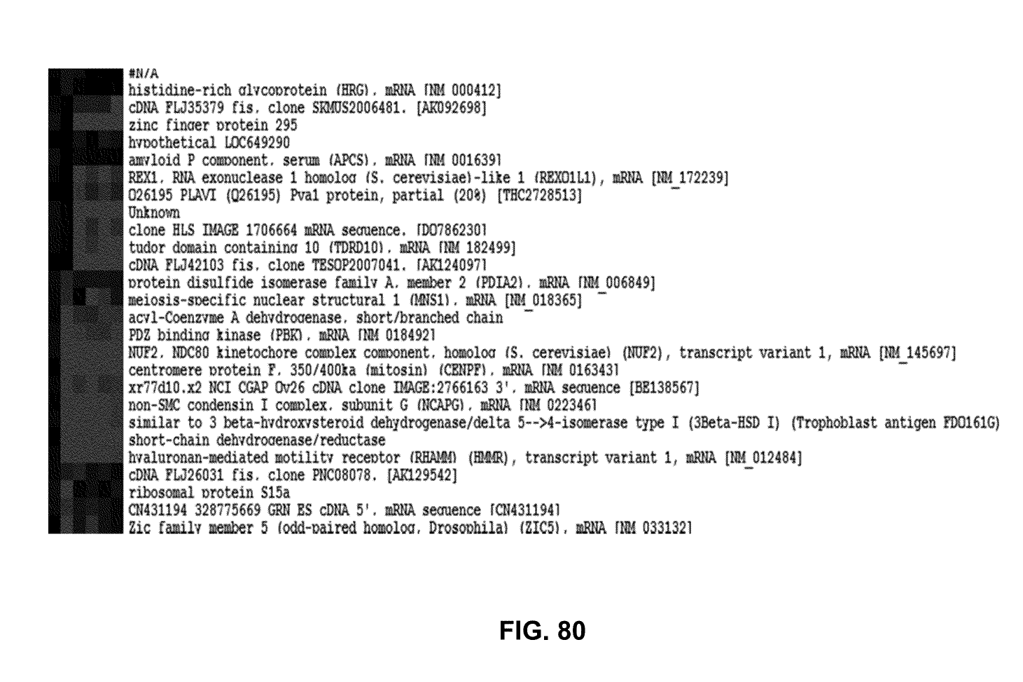

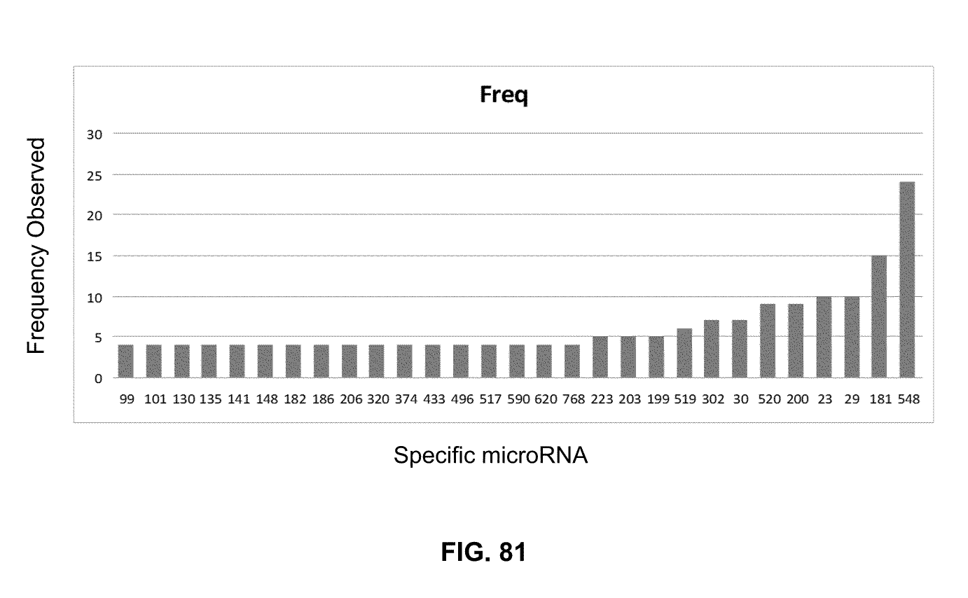

The most commonly overexpressed genes are shown in Table 3 above. A separate study was performed using the same platform as in Table 3 with exemplary results shown in Table 6. The top 100 overexpressed genes were identified, and of those, the genes overexpressed in at least 5 of 6 samples were determined and are shown in Table 7. An example of the results of the prostate cancer samples are shown in FIG. 80. Prostate cancer samples from 22 individuals were also examined by immunohistochemistry (IHC) for the overexpression of proteins. Those proteins that were overexpressed in at least 10 of the 22 samples are listed in Table 8.

TABLE 6

| | Gene | | | | | | |
|---|---|---|---|---|---|---|---|
| | Name | | 8617log2 | 8592log2 | 7419log2 | 7220log2 | 7193log2 |
| Gene | (Symbol) | 8548log2 Ratio | Ratio | Ratio | Ratio | Ratio | Ratio |
| 6- | zinc finger | 9.74543 | 6.56025 | −0.21638 | 10.1568 | 8.80924 | 8.44963 |
| phosphofructo- | protein | | | | | | |
| 2- | 295 | | | | | | |
| kinase/fructose- | (ZNF295) | | | | | | |
| 2,6- | | | | | | | |
| biphosphatase 3 | | | | | | | |
| (PFKFB3), | | | | | | | |
| mRNA | | | | | | | |
| [NM_004566] | | | | | | | |
| hyaluronan- | | 5.77984 | 7.42945 | 6.76902 | 8.47437 | 7.80986 | 7.83751 |
| mediated | | | | | | | |
| motility | | | | | | | |
| receptor | | | | | | | |
| (RHAMM) | | | | | | | |
| (HMMR), | | | | | | | |
| transcript | | | | | | | |
| variant 1, | | | | | | | |
| mRNA | | | | | | | |
| [NM_012484] | | | | | | | |
| cDNA | | 8.01354 | 7.86471 | −0.00877 | 8.61516 | 7.53774 | 6.75813 |
| FLJ42103 | | | | | | | |
| fis, clone | | | | | | | |
| TESOP2007041. | | | | | | | |
| [AK124097] | | | | | | | |
| asp | short- | 7.2929 | 7.11228 | 7.48869 | 8.17926 | 6.64991 | 6.50685 |
| (abnormal | chain | | | | | | |
| spindle) | dehydrogenase/ | | | | | | |
| homolog, | reductase | | | | | | |
| microcephaly | (MGC4172) | | | | | | |
| associated | | | | | | | |
| (Drosophila) | | | | | | | |
| (ASPM), | | | | | | | |
| mRNA | | | | | | | |
| [NM_018136] | | | | | | | |
| centromere | | 6.73987 | 6.92431 | 7.01586 | 8.49945 | 5.98086 | 6.04317 |
| protein F, | | | | | | | |
| 350/400ka | | | | | | | |
| (mitosin) | | | | | | | |
| (CENPF), | | | | | | | |
| mRNA | | | | | | | |
| [NM_016343] | | | | | | | |
| non-SMC | | 5.74212 | 6.48655 | 7.2098 | 8.10554 | 5.88082 | 5.89621 |
| condensin I | | | | | | | |
| complex, | | | | | | | |
| subunit G | | | | | | | |
| (NCAPG), | | | | | | | |
| mRNA | | | | | | | |
| [NM_022346] | | | | | | | |

TABLE 7

| Genes from Expression Profiling Array for | Frequency |
|---|---|
| 6 Prostate Cancer Samples | of Occurrence |
| NCAPG | 6/6 |
| CENPF | 6/6 |
| ASPM | 6/6 |
| cDNA FLJ42103 fis, | 5/6 |
| clone TESOP2007041 [AK124097] | |
| RHAMM (HMMR) | 5/6 |
| PFKFB3 | 5/6 |

TABLE 8

| Genes from IHC Analysis of | |
|---|---|
| 22 Prostate Cancer Samples | Frequency of Occurrence |
| Androgen Receptor | 18/22 |
| EGFR | 13/22 |
| HSP90 | 10/22 |
| SPARC | 10/22 |

### Example 23

### Generating Product Values for Characterizing Prostate Cancer

Patient healthcare can be greatly improved my providing improved methods of characterizing a disease or condition by providing a diagnosis, prognosis, or treatment selection for the disease or condition. The disease or condition can be detected earlier, or its stage determined to determine what type of treatment should be selected. The disease or condition can be a cancer, such as an epithelial cancer or carcinoma. There are different types of epithelial cells and these can develop into different types of cancer. For example, epithelial cells can constitute a flat surface covering of cells called squamous cells. Additionally, epithelial cells can take a glandular form called adenomatous cells. Also, epithelial cells can form a stretchy layer called transitional cells. Carcinomas make up about 85% of all cancers, and include breast, prostate, lung, colorectal, bladder and ovarian cancers.

Epithelial based cancers usually result in a solid mass or a tumor from which cancer cells migrate throughout the body eventually residing in other locations to establish secondary tumors or metastases. One of the major therapies for cancers resulting in solid tumors is the surgical removal or oblation of the tumor by physical or chemical means. After a cancer is removed from a subject, for example by surgical removal, the monitoring or detection of recurrence of the cancer at the same or secondary sites, can be indicated, so that additional therapies can be employed for treatment should that occur. Likewise, some means of monitoring the success of cancer therapy can be indicated during the treatment phase in order to determine if the therapy is being successful or not and in order to appropriately adapt the therapy accordingly.

There is a need for methods of characterizing cancers, such as epithelial cancers. For example, despite the contribution that the Prostate Specific Antigen (PSA) test has made to the management of prostate cancer, it is plagued by significant shortcomings which result from the antigen being specific for prostate tissue and not for prostate cancer. While the test is highly specific for the PSA antigen, not all prostate cancers release excessive levels of the antigen into the serum. This results in the lack of clinical sensitivity and results in frequent missing of clinically significant cancers with routine PSA examinations.

A normal PSA value is currently considered to be less than 4.0 ng/mL. It is believed that at least 20% of men with significant prostate cancers may have a PSA value less than 4.0 ng/mL. However, since PSA is made by normal, indolent hyperplastic, pre-malignant and malignant tissue, the finding of an elevated PSA (greater than 4.0 ng/mL) does not always indicate cancer. If the serum PSA is in the range of 4.0 to 10 ng/mL there is only a 25-30% chance of finding prostate cancer even through the use of repeated and more thorough biopsies (10-12 cores). The finding of an elevated PSA value frequently results in the subject undergoing an uncomfortable and potentially dangerous transrectal biopsy. It is not uncommon for a man with a significantly elevated PSA to undergo two or more biopsies, in an attempt to find the cause of the elevated serum PSA. This Example improves upon PSA testing by combining such values with microRNA analysis.

A product value was determined by combining of miR-141 values with PSA values obtained from a subject's blood sample to create a product value used to detect prostate cancer. Data on the serum PSA levels and miR-141 levels from 25 men with metastatic prostate cancer and from 25 normal men was obtained from Mitchell et al., PNAS Jul. 29, 2008 Vol 105 No. 30 p. 10513-10518. The product value was determined by multiplying the miR-141 copy number by the PSA level (Table 9).

TABLE 9

| Normal | | | | Prostate Cancer | | | |
|---|---|---|---|---|---|---|---|
| Patient | | | Product 141 × | Patient | miR-141 | | Product 141 × |
| id | miR-141 copies | PSA | PSA | id | copies | PSA | PSA |
| 1 | 83 | 0.2 | 17 | 26 | 1503 | 1.57 | 2360 |
| 2 | 69 | 0.56 | 39 | 27 | 937 | 2.54 | 2380 |
| 3 | 113 | 0.73 | 82 | 28 | 574 | 4.66 | 2675 |
| 4 | 215 | 0.42 | 90 | 29 | 1031 | 9.17 | 9454 |
| 5 | 292 | 0.31 | 91 | 30 | 833 | 11.48 | 9563 |
| 6 | 308 | 0.35 | 108 | 31 | 476 | 45.16 | 21496 |
| 7 | 615 | 0.2 | 123 | 32 | 521 | 46.78 | 24372 |
| 8 | 145 | 0.92 | 133 | 33 | 224 | 171.8 | 38483 |
| 9 | 169 | 0.89 | 150 | 34 | 5004 | 23.04 | 115292 |
| 10 | 201 | 0.92 | 185 | 35 | 866 | 170 | 147220 |
| 11 | 603 | 0.32 | 193 | 36 | 4453 | 34 | 151402 |
| 12 | 252 | 0.86 | 217 | 37 | 761 | 215.4 | 163919 |
| 13 | 361 | 0.65 | 235 | 38 | 5505 | 37.35 | 205612 |
| 14 | 621 | 0.42 | 261 | 39 | 46125 | 38 | 1752750 |
| 15 | 417 | 0.76 | 317 | 40 | 2985 | 680.3 | 2030696 |
| 16 | 373 | 0.9 | 336 | 41 | 9309 | 345.9 | 3219983 |
| 17 | 731 | 0.5 | 366 | 42 | 7493 | 432 | 3236976 |
| 18 | 414 | 1.03 | 426 | 43 | 3404 | 963 | 3278052 |
| 19 | 468 | 0.93 | 435 | 44 | 12055 | 500 | 6027500 |
| 20 | 445 | 1.03 | 458 | 45 | 11113 | 912 | 10135056 |
| 21 | 788 | 0.68 | 536 | 46 | 11727 | 1272 | 14916744 |
| 22 | 598 | 1.08 | 646 | 47 | 20585 | 2151 | 44278335 |
| 23 | 1771 | 0.58 | 1027 | 48 | 89811 | 3647.4 | 327576641 |
| 24 | 1459 | 0.79 | 1153 | 49 | 75267 | 5974 | 449645058 |
| 25 | 2508 | 0.54 | 1354 | 50 | 78639 | 8420 | 662140380 |

The mean number of copies per microliter of serum of miR-141 from the men with prostate cancer is 15,648 with a 95% confidence interval about the mean of +/−10,431 copies per microliter. The mean number of copies per microliter of serum of miR-141 from men without prostate cancer is 560 with a 95% confidence interval of the mean of +/−223 copies per microliter (Table 10). There is a clear differentiation of men with prostate cancer from normal men without prostate cancer.

TABLE 10

| | Normals | N = 25 | | | | | PrCa | N = 25 | | | |
|---|---|---|---|---|---|---|---|---|---|---|---|
| miR-141 | MEAN | SDV | 95% CIM | upper | lower | miR-141 | MEAN | SDV | 95% CIM | upper | lower |
| | 560.76 | 569.0021 | 223.0447 | 783.8047 | 337.7153 | | 15648.04 | 26612.09 | 10431.75 | 26079.79 | 5216.293 |
| | Normals | | | | | | PrCa | | | | |
| PSA | MEAN | SDV | 95% CIM | upper | lower | PSA | MEAN | SDV | 95% CIM | upper | lower |
| | 0.6628 | 0.271562 | 0.10645 | 0.76925 | 0.55635 | | 1044.342 | 2059.117 | 807.1591 | 1851.501 | 237.1829 |

The product value provides a novel analysis of data by using the number of miR-141 copies and the PSA values for a subject that is predictive of prostate cancer. The product value separates the men with prostate cancer from the men without prostate cancer with 100% sensitivity and 100% specificity.

Although preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of characterizing prostate cancer in a human subject comprising: determining the level or presence of a PCSA polypeptide in a biological sample from a subject and comparing the level or presence to a reference, wherein a difference in the level or presence as compared to the reference is used to characterize the prostate cancer.

2. The method of claim 1, wherein the characterizing comprises a characterization selected from the group consisting of diagnosis, prognosis, staging, grading, determination of drug efficacy, monitoring the status of the subject's response or resistance to a treatment, selection of a treatment for the prostate cancer, and a combination thereof.

3. The method of claim 2, wherein the subject is non-responsive to a current therapeutic being administered to the subject.

4. The method of claim 3, wherein the therapeutic is a cancer therapeutic.

5. The method of claim 1, wherein the reference is derived from a subject without cancer.

6. The method of claim 1, wherein the reference is derived from the subject over a time course.

7. The method of claim 1, wherein the biological sample comprises a bodily fluid.

8. The method of claim 1, wherein the bodily fluid comprises a bodily fluid selected from the group consisting of peripheral blood, serum, plasma, urine, semen, prostatic fluid, cowper's fluid, pre-ejaculatory fluid, and a combination thereof.

9. The method of claim 1, wherein the bodily fluid comprises plasma or serum.

10. The method of claim 1, further comprising determining the presence or level of at least one protein and/or nucleic acid molecule in the biological sample.

11. The method of claim 10, wherein the at least one protein is selected from EpCAM, CD9, CD63, CD81, PSMA, a BCNP protein biomarker, a metalloproteinase protein biomarker and a combination thereof.

12. The method of claim 10, wherein the at least one nucleic acid molecule is an RNA or DNA molecule.

13. The method of claim 1 or 11, wherein at least one membrane vesicle is isolated from the biological sample prior to the determining.

14. The method of claim 13, wherein the at least one membrane vesicle comprises at least one membrane vesicles with a diameter of about 30 nm to about 800 nm.

15. The method of claim 13, wherein the at least one vesicles comprises at least one membrane vesicles with a diameter of about 30 nm to about 200 nm.

16. The method of claim 13, wherein the at least one membrane vesicle is isolated from the biological sample using an isolation technique selected from the group consisting of size exclusion chromatography, density gradient centrifugation, differential centrifugation, nanomembrane ultrafiltration, immunoabsorbent capture, affinity purification, affinity selection, microfluidic separation, and a combination thereof.

17. The method of claim 16, wherein the isolating comprises contacting the biological sample with at least one binding agent that is specific for the PCSA polypeptide, thereby binding at least one membrane vesicle associated with the PCSA polypeptide.

18. The method of claim 13, wherein the at least one nucleic acid molecule comprises a microRNA.

19. The method of claim 17, wherein the at least one binding agent is selected from the group consisting of a DNA molecule, RNA molecule, antibody, antibody fragment, aptamer, peptoid, zDNA, peptide nucleic acid (PNA), locked nucleic acids (LNA), lectin, peptide, dendrimer, chemical compound, and a combination thereof.

20. The method of claim 17, wherein the at least one binding agent is selected from the group consisting of PSA, PSMA, mAB 5D4, XPSM-A9, XPSM-A10, Galectin-3, E-selectin, Galectin-1, E4 (IgG2a kappa), and a combination thereof.

21. The method of claim 18, wherein the microRNA is selected from the group consisting of mir-1, let-7b, mir-21, mir25, mir-32, mir-93, mir-96, mir-141, mir-143, mir-145, mir-182, mir-183, mir-221, mir-222, mir-301, mir-375, and a combination thereof.
